(19) United States
(12) Patent Application Publication
(10) Pub. No.: US 2024/0132525 A1
(43) Pub. Date: Apr. 25, 2024

(54) BORON CONTAINING PYRIMIDINE COMPOUNDS, COMPOSITIONS COMPRISING THEM, METHODS AND USES THEREOF

(71) Applicants: BOEHRINGER INGELHEIM ANIMAL HEALTH USA INC., DULUTH, GA (US); BORAH, INC., NASHVILLE, TN (US)

(72) Inventors: ALAN LONG, WARTHAUSEN (DE); SHON R. PULLEY, TRENTON, SC (US); KEITH ANDREW NEWTON GRAHAM, BERLIN (DE); CHUNLIANG LIU, CARY, NC (US); YASHEEN ZHOU, MORAGA, CA (US)

(21) Appl. No.: 18/210,783

(22) Filed: Jun. 16, 2023

Related U.S. Application Data
(60) Provisional application No. 63/366,535, filed on Jun. 17, 2022.

(51) Int. Cl. C07F 5/02

(52) U.S. Cl. CPC C07F 5/027

(57) ABSTRACT

The present disclosure describes boron containing pyrimidine compounds, or their pharmaceutically acceptable salts, pharmaceutical compositions containing them, and their medical uses. The compounds of the present disclosure have activity as Janus kinase (JAK) inhibitors and are useful in the in the treatment or control of inflammation, auto-immune diseases, cancer, and other disorders and indications where modulation of JAK would be desirable. Also described are methods of treating inflammation, auto-immune diseases, cancer, and other conditions that are susceptible to the inhibition of a Janus kinase by administering a compound herein described.

### BACKGROUND OF THE DISCLOSURE

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation, or de-regulation, as well as over- or under-production of growth factors or cytokines has been implicated in many diseases, including but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, survival, apoptosis, mitogenesis, cell cycle control, and cell mobility implicated in the aforementioned and related diseases. Thus, protein kinases have emerged as an important class of enzymes as targets for therapeutic intervention. In particular, the JAK family of cellular protein tyrosine kinases (JAK-1, JAK-2, JAK-3, and Tyk-2) play a central role in cytokine signaling (Kisseleva et al, Gene, 2002, 285, 1; Yamaoka et al. Genome Biology 2004, 5, 253)). Upon binding to their receptors, cytokines activate JAK, which then phosphorylate the cytokine receptor, thereby creating docking sites for signaling molecules, notably, members of the signal transducer and activator of transcription (STAT) family that ultimately lead to gene expression, which stimulates biologic responses such as an itch signal. Activation of the JAK-STAT pathway also results in several other ancillary biologic activities that contribute to the inflammation and pruritic processes that contribute to acute allergy in animals but can also exacerbate clinical signs and contribute to chronic allergy.

Atopic dermatitis (AD), also known as eczema, is a common chronic inflammatory skin disease, affecting approximately 20% of children and up to 10% of adults and it imposes a significant financial and societal burden because of the direct medical costs and decreased productivity of individuals with AD. The burden of AD appears to be related mainly to the limited methods of treatment. Furthermore, according to the AD treatment guidelines, there is no standard of care and treatment may be tailored to an individual's needs. Topical interventions are the mainstay of AD therapy. Until now, topical corticosteroids have been the first-line treatment. Their use, however, may be limited by potential local and systemic adverse effects. Topical calcineurin inhibitors are classified as second-line anti-inflammatory therapy for AD, with advantages in long-term maintenance and application to special sites. Topical calcineurin inhibitors inhibit calcineurin-dependent T-cell activation; however, a black box warning about the potential for developing malignant neoplasms with the use of topical calcineurin inhibitors reduces patients' adherence to treatment.

Psoriasis and psoriatic arthritis are associated with aberrant inflammation and the production of proinflammatory mediators. Psoriasis and psoriatic arthritis are inflammatory diseases with overlapping features and shared immunologic mechanisms. Psoriasis is a systemic disease in that it primarily affects the skin but up to 40% of individuals with psoriasis may go on to develop psoriatic arthritis. Psoriatic arthritis typically affects the peripheral joints and may occasionally affect the spine and sacroiliac area. Enthesitis, dactylitis, and nail changes such as pitting and discoloration are also common manifestations of psoriatic disease in patients with joint involvement.

Pruritus is commonly a significant clinical sign associated with flea associated dermatitis in dogs. Medical management of for pruritis may be sought in cases where the cause of itching is not identifiable, or treatment of underlying disease does not eliminate itching. However, control of itching with antihistamines is usually ineffective, and while treatment with glucocorticoids can be effective, long term use is not ideal due to adverse side effects including excessive hunger, thirst, and urination, and increased risk of diabetes and urinary tract infections.

JAK inhibition may provide a therapeutic strategy for various immune and inflammatory diseases, including rheumatoid arthritis (RA), arthritis, ulcerative colitis, Crohn's disease, inflammatory bowel disease (IBD), psoriasis, alopecia areata, atopic dermatitis, vitiligo, palmoplantar pustulosis, mucocutaneous disease erythema multiforme, mycosis fungoides, graft-versus-host disease, cutaneous lupus, transplant rejection, systemic lupus erythematosus (SLE), dermatomyositis, Sjogren's syndrome, dry eye disease, secondary hypereosinophilic syndrome (HES), allergy, allergic dermatitis, asthma, vasculitis, multiple sclerosis, diabetic nephropathy, cardiovascular disease, artherosclerosis, and cancer. Reference is made to Schwartz et al., JAK inhibition as a therapeutic strategy for immune and inflammatory diseases, Nat Rev Drug Discov., 2017 Dec. 28., 17(1):78, herein incorporated by reference with regard to the rationale for targeting JAKs.

Various classes of compounds have been shown to inhibit JAK enzymes. For example, U.S. Pat. No. 8,133,899 B2 (to Pfizer) discloses use of pyrrolo[2,3-D]pyrimidine compounds as JAK inhibitors. In particular, oclacitinib (APOQUEL®) is a cyclohexylamino pyrrolopyrimidine demonstrated to be a Janus kinase inhibitor that controls clinical signs of allergic skin disease in dogs (as disclosed in J. Vet. Pharmacol. Therap. 2014 August 37(4): 317-324). JAK inhibitor compounds are described in published patent application nos. US 2020/0339585, WO 2009/114512 A1, WO 2021/003501 A1 and U.S. Pat. No. 7,598,257 B2.

Published patent applications filed by Merck Sharp & Dohme Corporation and Intervet Inc. relating to pyrazole carboxamide compounds as JAK inhibitors include WO 2013/041042 A1, WO 2018/108969 A1, WO 2020/118597 A1, WO 2020/120673 A1, WO 2020/120679 A1, and WO2020221914 A1 (all incorporated by reference herein in their entirety).

Pyrazole carboxamide Janus Kinase 1 inhibitors are also described by Siu et al., The Discovery of 3-((4-Chloro-3-methoxyphenyl)amino)-1-((3R,4S)-4-cyanotetrahydro-2H pyran-3-yl)-1H-pyrazole-4-carboxamide, a Highly Ligand Efficient and Efficacious Janus Kinase 1 Selective Inhibitor with Favorable Pharmacokinetic Properties, J. Med. Chem. 2017 Dec. 14; 60(23): 9676-9690 (incorporated herein by reference).

There remains a need for therapies targeting and modulating JAK kinases for the treatment or control of inflammation, auto-immune diseases, cancer, and other disorders and indications where modulation of JAK modulation would be desirable.

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference.

Citation or identification of any document in this application is not an admission that such document is available as prior art.

### SUMMARY OF THE DISCLOSURE

The present disclosure provides boron-containing compounds, or their pharmaceutically acceptable salts, pharmaceutical compositions containing them, and their medical uses. The compounds of the disclosure have activity as Janus kinase (JAK) inhibitors and are useful in the treatment or control of inflammation, auto-immune diseases, cancer, and other disorders and indications where modulation of JAK would be desirable. Also described herein are methods of treating inflammation, auto-immune diseases, cancer, and other conditions susceptible to inhibition of JAK by administering a compound of the disclosure.

In a first aspect, the present disclosure provides for boron containing pyrimidine compounds, or their pharmaceutically acceptable salts, pharmaceutical compositions containing them, and their medical uses. The compounds of the disclosure have activity as Janus kinase (JAK) inhibitors and are useful in the treatment or control of inflammation, auto-immune diseases, cancer, and other disorders and indications where modulation of JAK would be desirable.

In one embodiment, the present disclosure provides a compound of formula (I), (Ia) or (Ib), or a pharmaceutically acceptable salt or a stereoisomer or a tautomer thereof; wherein A is selected from the group consisting of benzo[c][1,2]oxaborol-1(3H)-ol, 3,4-dihydro-1Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇ as defined herein, and the variables R¹⁻¹¹, R^{11a}, and R¹²⁻¹³ in respect of compounds of formula (I), (Ia) and (Ib) are defined herein.

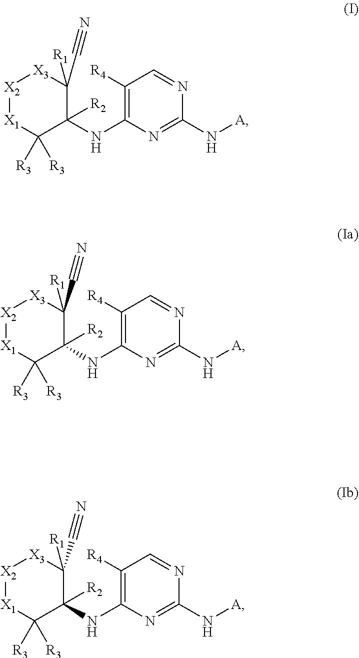

One embodiment of the present disclosure includes a method for treating a patient having a disease or disorder susceptible to modulation of JAK including administering a therapeutically effective amount of a compound of the present disclosure. In one aspect, the disease or disorder is one or more of atopic dermatitis, flea allergy dermatitis, eczema, pruritus, psoriasis, psoriatic arthritis, Bechet's disease, pityriasis rubra pilaris, alopecia areata, discoid lupus erythematosus, vitiligo, palmoplantar pustulosis, mucocutaneous disease erythema multiforme, mycosis fungoides, graft-versus-host disease, cutaneous lupus, rheumatoid arthritis (RA), arthritis, ulcerative colitis, Crohn's disease, inflammatory bowel disease (IBD), transplant rejection, systemic lupus erythematosus (SLE), dermatomyositis, Sjogren's syndrome, dry eye disease, secondary hypereosinophilic syndrome (HES), allergy, allergic dermatitis, asthma, vasculitis, multiple sclerosis, diabetic nephropathy, cardiovascular disease, artherosclerosis, and cancer. In one aspect, the disease or disorder is one or more of atopic dermatitis, flea allergy dermatitis, psoriasis, and rheumatoid arthritis. In one aspect, the compound is administered in an amount to perturb an immune regulatory pathway in a cell. In one aspect, the perturbation results in an effect on the JAK-STAT pathway.

One embodiment of the present disclosure includes a method of inhibiting JAK in a mammalian cell including contacting the mammalian cell with a compound of the present disclosure. In one aspect, the mammalian cell is a cell from a subject having an inflammatory condition.

One embodiment of the present disclosure includes a composition including a compound of the present disclosure and a pharmaceutically or veterinary acceptable carrier.

One embodiment of the present disclosure includes a combination including a compound of the present disclosure, and one or more other pharmaceutical or veterinary active substances.

In another aspect, the present disclosure provides methods of treating inflammation, auto-immune diseases, cancer, and other conditions susceptible to inhibition of JAK by administering a compound herein described.

In one embodiment of the present disclosure, a method for treating one or more diseases or disorders of inflammation, auto-immune dysfunction, and cancer is provided including administering to a subject in need thereof an effective amount of a compound of the present disclosure. In one aspect, the disease or disorder is atopic dermatitis, flea allergy dermatitis, psoriasis, or rheumatoid arthritis. In one embodiment, the subject is a mammal. In one embodiment, the subject is a non-human animal. In one embodiment, the subject is selected from livestock mammals, domestic mammals, or companion animals. In one aspect, the subject is selected from cattle, sheep, goats, llamas, alpacas, pigs, horses, donkeys, dogs, and cats. In one aspect, the subject is a human.

One embodiment of the present disclosure includes a compound of the present disclosure for use in medicine.

One embodiment of the present disclosure includes a compound of the present disclosure for the manufacture of a medicament for the treatment of one or more diseases or disorder of inflammation, auto-immune dysfunction, and cancer. In one aspect, the disease or disorder is atopic dermatitis, psoriasis, or rheumatoid arthritis.

One embodiment of the present disclosure includes a use of a compound of the present disclosure for the treatment of one or more diseases or disorders of inflammation, auto-immune dysfunction, and cancer. In one aspect, the disease or disorder is atopic dermatitis, psoriasis, or rheumatoid arthritis.

One or more aspects and embodiments may be incorporated in a different embodiment although not specifically described. That is, all aspects and embodiments may be combined in any way or combination.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to the described variable.

These and other embodiments are disclosed or are obvious from and encompassed by the following Detailed Description.

### DETAILED DESCRIPTION

In a first aspect, the present disclosure provides for boron containing pyrimidine compounds, or their pharmaceutically acceptable salts, pharmaceutical compositions containing them, and their medical uses.

The compounds of the disclosure have activity as Janus kinase (JAK) inhibitors and are useful in the treatment or control of inflammation, auto-immune diseases, cancer, and other disorders and indications where modulation of JAK would be desirable.

In one embodiment, the present disclosure provides for a compound of formula (I), (Ia), or (Ib), or a pharmaceutically acceptable salt, or a stereoisomer or a tautomer thereof:

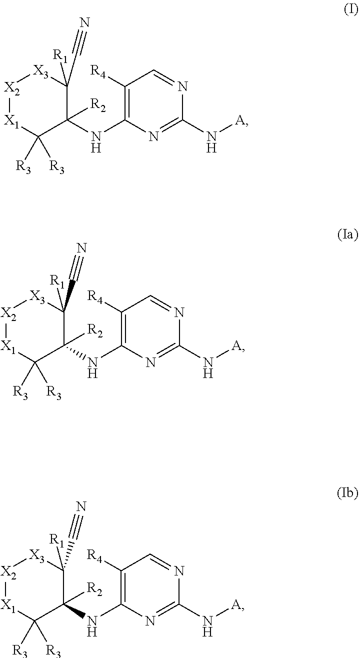

wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R₁ is selected from the group consisting of hydrogen, halogen (when X₃ is —C(R⁶)(R⁷)), hydroxy, C₁₋₆ alkyl, C₃₋₇ cycloalkyl, C₁₋₆ alkyl(oxy), and C₃₋₇ cycloalkyl(oxy), wherein the last four members of the above-mentioned R¹ group may optionally be substituted independently of one another with one or more halogen;
R₂ is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R₃ is independently selected from the group consisting of hydrogen, halogen (when X₁ is —C(R⁶)(R⁷)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of: hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl,
A is selected from the group consisting of benzo[c][1,2]oxaborol-1(3H)-ol, 3,4-dihydro-1Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇:

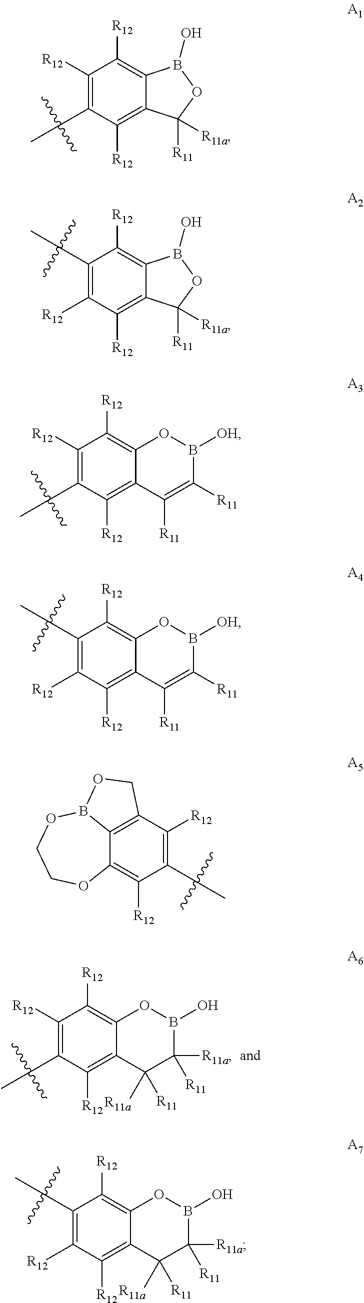

R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇ cycloalkyl, C₁₋₆ alkyl(oxy), C₃₋₇ cycloalkyl(oxy), COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl); wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

In one embodiment of the compound of formula I as described herein
X₁ is —C(R⁶)(R⁷)—;
X₂ is selected from —C(R⁶)(R⁷)—, or X₂ comprises a bond between X₁ and X₃;
X₃ is-C(R⁶)(R⁷)—
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of: hydrogen, halogen, C₁-C₃ alkyl, and C₁-C₃ haloalkyl;
R⁶ and R⁷ are independently selected from the group consisting of hydrogen, —C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring;
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

In yet another embodiment of the compound of formula I as described herein
X₁ is selected from the group consisting of —O—, and —C(R⁶)(R⁷)—;
X₂ is selected from the group consisting of —O—, and —C(R⁶)(R⁷)—, or
   X₂ comprises a bond between X₁ and X₃;
X₃ is —C(R⁶)(R⁷)—, except X₁ and X₂ cannot both be —O—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of: hydrogen, halogen, C₁-C₃ alkyl, and C₁-C₃ haloalkyl;
R⁶ and R⁷ are independently selected from the group consisting of hydrogen, —C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring;
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

In one embodiment of the compound of formula I as described herein
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

In one embodiment of the compound of formula I as described herein
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

In one embodiment, the present disclosure provides for a compound of formula (Ia) or (Ib), or a pharmaceutically acceptable salt, or a stereoisomer or a tautomer thereof, wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or
X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R¹ is selected from the group consisting of hydrogen, halogen (when X₃ is —C(R⁷)(R⁸)—), hydroxy, C₁₋₆ alkyl, C₃₋₇ cycloalkyl, C₁₋₆ alkyl(oxy), and C₃₋₇ cycloalkyl(oxy), wherein the last four members of the of the above-mentioned R¹ group may optionally be substituted independently of one another with one or more halogen;
R² is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R³ is independently selected from the group consisting of hydrogen, halogen (when X₁ is —C(R⁷)(R⁸)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl;
A is selected from the group consisting of benzo[c][1,2]oxaborol-1(3H)-ol, 3,4-dihydro-1Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇:

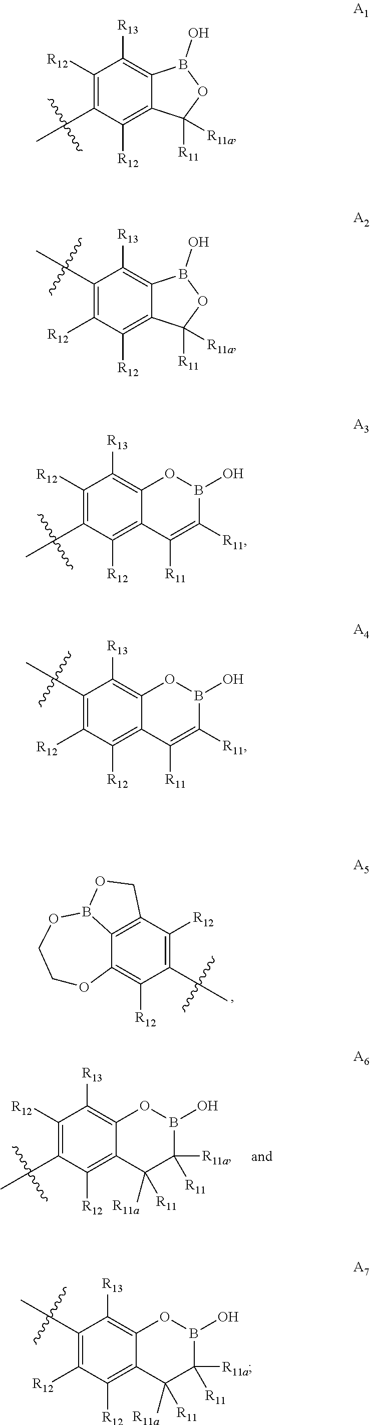

R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇ cycloalkyl, C₁₋₆ alkyl(oxy), C₃₋₇ cycloalkyl(oxy), COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C3-6-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

In one embodiment of the compound of formula (Ia) or (Ib) as described herein
X₁ is —C(R⁶)(R⁷)—;
X₂ is selected from —C(R⁶)(R⁷)—, or X₂ comprises a bond between X₁ and X₃;
X₃ is-C(R⁶)(R⁷)—
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of hydrogen, halogen, C₁-C₃ alkyl, and C₁-C₃ haloalkyl;
R⁶ and R⁷ are independently selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring; and
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

In another embodiment of the compound of formula (Ia) or (Ib) as described herein
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R₄ is selected from the group consisting of hydrogen, halogen, C₁-C₃ alkyl, and C₁-C₃ haloalkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

In yet another embodiment of the compound of formula (Ia) or (Ib) as described herein
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

In yet another embodiment of the compound of formula (Ia) or (Ib) as described herein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or
   X₂ comprises a bond between X₁ and X₃;
X₃ is —C(R⁶)(R⁷)—, except X₁ and X₂ cannot both be —O—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned group may optionally be substituted independently of one another with one or more halogen;
R₄ is selected from the group consisting of halogen, C₁-C₃ alkyl, and C₁-C₃ haloalkyl;
R⁶ and R⁷ are independently selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned group may optionally be substituted independently of one another by one or more substituents selected from the group consisting of halogen and hydroxy, and R⁶ and R⁷ may be linked together to form a ring; and
each R¹² is independently selected from the group consisting of hydrogen, halogen, and C₁₋₆ alkyl.

In another embodiment of the compound of formula (Ia) or (Ib) as described herein
X₁ is selected from the group consisting of —O—, —N(R⁵)—, and —C(R⁶)(R⁷)—;
X₂ is selected from the group consisting of —O— and —C(R⁶)(R⁷)—, except X₁ and X₂ may not both be —O—, or X₂ comprises a bond between X₁ and X₃;
X₃ is —C(R⁶)(R⁷)—;
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl is optionally substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members said R¹³ group may optionally be substituted independently of one another by one or more substituents selected from the group consisting of halogen and hydroxy.

In another embodiment of the compound of formula (Ia) or (Ib) as described herein
X₂ is —C(R⁶)(R⁷)—;
X₃ is —C(R⁶)(R⁷)—;
R¹, R², and R³, are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl is optionally substituted with one or more of halogen;
each R¹¹ is independently selected from the group consisting of hydrogen, halogen, C₁₋₆ alkyl, and C₃₋₇ cycloalkyl, wherein the last two members of the above-mentioned R¹¹ group may optionally be substituted independently of one another with one or more halogen;
each R^{11a} is independently selected from the group consisting of hydrogen, halogen, C₁₋₆ alkyl, and C₃₋₇ cycloalkyl, wherein the last two members of the above-mentioned R¹¹ group may optionally be substituted independently of one another with one or more halogen;
each R¹² is hydrogen; and
each R¹³ is independently selected from the group consisting hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

In one embodiment, the present disclosure is a compound selected from the group shown in Table 1 below:

TABLE 1

| Example | |
|---|---|
| Number | Structure |
| 1 | 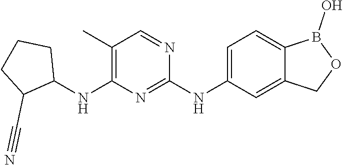 [SMILES: Cc1cnc(Nc2ccc3c(c2)COB3O)nc1NC1CCCC1C#N] |
| 2 | 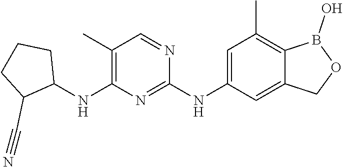 [SMILES: Cc1cnc(Nc2cc(C)c3c(c2)COB3O)nc1NC1CCCC1C#N] |
| 3 | 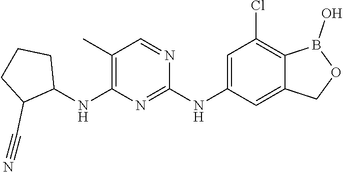 [SMILES: Cc1cnc(Nc2cc(Cl)c3c(c2)COB3O)nc1NC1CCCC1C#N] |
| 4 | 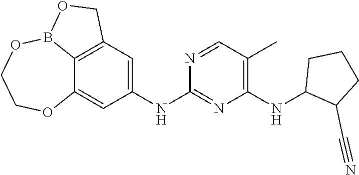 [SMILES: Cc1cnc(Nc2cc3c4c(c2)OCCOB4OC3)nc1NC1CCCC1C#N] |
| 5 | 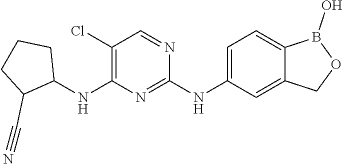 [SMILES: N#CC1CCCC1Nc1nc(Nc2ccc3c(c2)COB3O)ncc1Cl] |
| 6 | 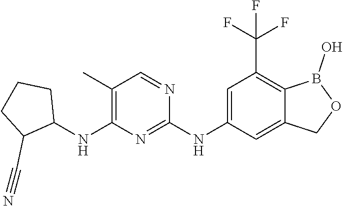 [SMILES: Cc1cnc(Nc2cc3c(c(C(F)(F)F)c2)B(O)OC3)nc1NC1CCCC1C#N] |
| 7 | 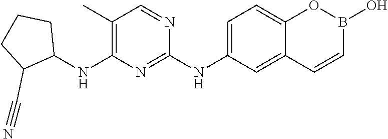 [SMILES: Cc1cnc(Nc2ccc3c(c2)C=CB(O)O3)nc1NC1CCCC1C#N] |
| 8 | 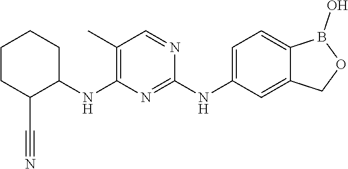 [SMILES: Cc1cnc(Nc2ccc3c(c2)COB3O)nc1NC1CCCCC1C#N] |
| 9 | 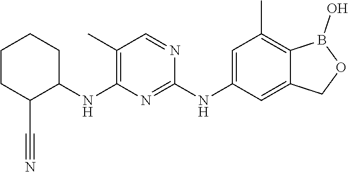 [SMILES: Cc1cnc(Nc2cc(C)c3c(c2)COB3O)nc1NC1CCCCC1C#N] |
| 10 | 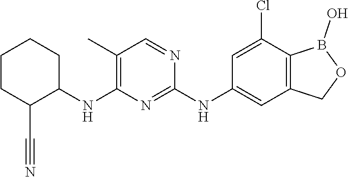 [SMILES: Cc1cnc(Nc2cc(Cl)c3c(c2)COB3O)nc1NC1CCCCC1C#N] |
| 11 | 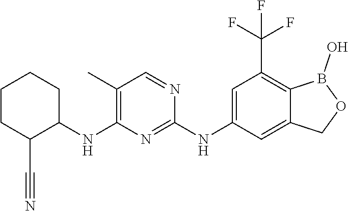 [SMILES: Cc1cnc(Nc2cc3c(c(C(F)(F)F)c2)B(O)OC3)nc1NC1CCCCC1C#N] |
| 12 | 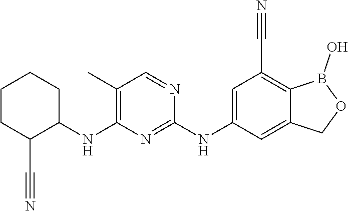 [SMILES: Cc1cnc(Nc2cc(C#N)c3c(c2)COB3O)nc1NC1CCCCC1C#N] |
| 13 | 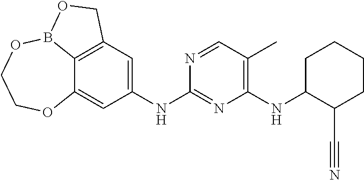 [SMILES: Cc1cnc(Nc2cc3c4c(c2)OCCOB4OC3)nc1NC1CCCCC1C#N] |
| 14 | 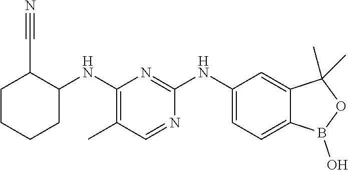 [SMILES: Cc1cnc(Nc2ccc3c(c2)C(C)(C)OB3O)nc1NC1CCCCC1C#N] |
| 15 | 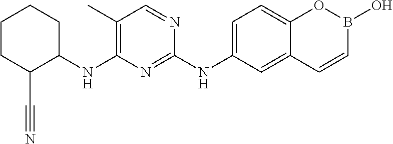 [SMILES: Cc1cnc(Nc2ccc3c(c2)C=CB(O)O3)nc1NC1CCCCC1C#N] |
| 16 | 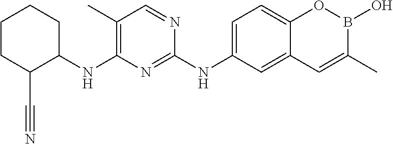 [SMILES: CC1=Cc2cc(Nc3ncc(C)c(NC4CCCCC4C#N)n3)ccc2OB1O] |
| 17 | 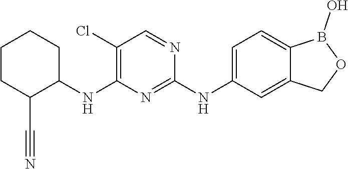 [SMILES: N#CC1CCCCC1Nc1nc(Nc2ccc3c(c2)COB3O)ncc1Cl] |
| 18 | 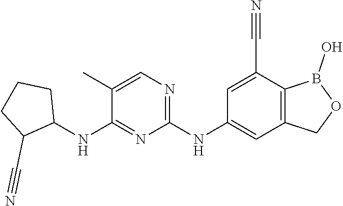 [SMILES: Cc1cnc(Nc2cc(C#N)c3c(c2)COB3O)nc1NC1CCCC1C#N] |
| 19 | 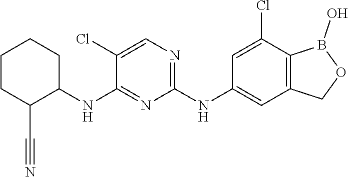 [SMILES: N#CC1CCCCC1Nc1nc(Nc2cc(Cl)c3c(c2)COB3O)ncc1Cl] |
| 20 | 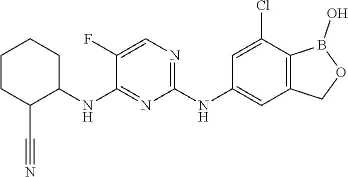 [SMILES: N#CC1CCCCC1Nc1nc(Nc2cc(Cl)c3c(c2)COB3O)ncc1F] |
| 21 | 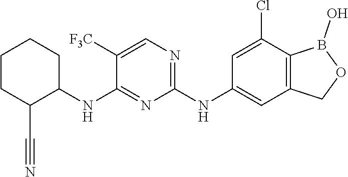 [SMILES: N#CC1CCCCC1Nc1nc(Nc2cc(Cl)c3c(c2)COB3O)ncc1C(F)(F)F] |
| 22 | 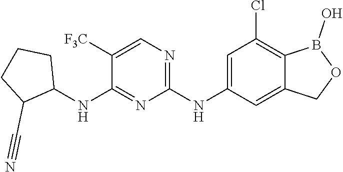 [SMILES: N#CC1CCCC1Nc1nc(Nc2cc(Cl)c3c(c2)COB3O)ncc1C(F)(F)F] |
| 23 | 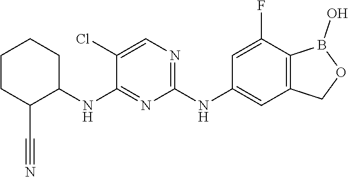 [SMILES: N#CC1CCCCC1Nc1nc(Nc2cc(F)c3c(c2)COB3O)ncc1Cl] |
| 24 | 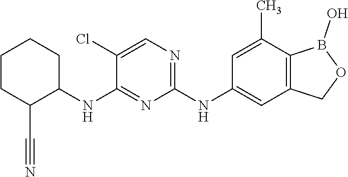 [SMILES: Cc1cc(Nc2ncc(Cl)c(NC3CCCCC3C#N)n2)cc2c1B(O)OC2] |

In one embodiment, the present disclosure is a compound selected from the group shown in Table 2 below:

TABLE 2

| Example | |
|---|---|
| Number | Structure |
| 25 | 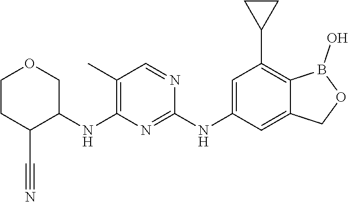 [SMILES: Cc1cnc(Nc2cc3c(c(C4CC4)c2)B(O)OC3)nc1NC1COCCC1C#N] |
| 26 | 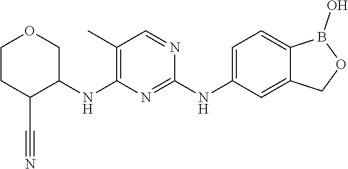 [SMILES: Cc1cnc(Nc2ccc3c(c2)COB3O)nc1NC1COCCC1C#N] |
| 27 | 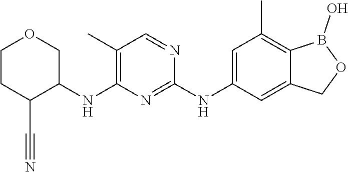 [SMILES: Cc1cnc(Nc2cc(C)c3c(c2)COB3O)nc1NC1COCCC1C#N] |
| 28 | 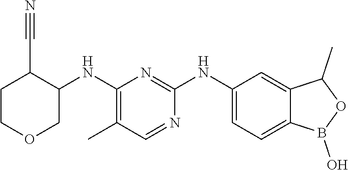 [SMILES: Cc1cnc(Nc2ccc3c(c2)C(C)OB3O)nc1NC1COCCC1C#N] |
| 29 | 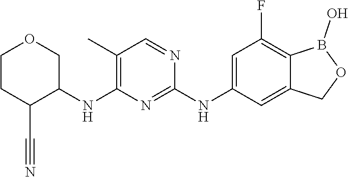 [SMILES: Cc1cnc(Nc2cc(F)c3c(c2)COB3O)nc1NC1COCCC1C#N] |
| 30 | 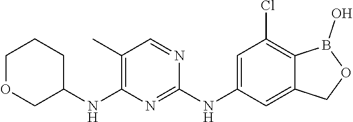 [SMILES: Cc1cnc(Nc2cc(Cl)c3c(c2)COB3O)nc1NC1CCCOC1] |
| 31 | 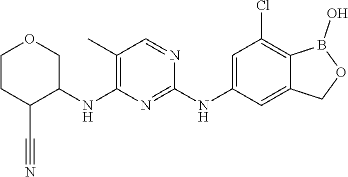 [SMILES: Cc1cnc(Nc2cc(Cl)c3c(c2)COB3O)nc1NC1COCCC1C#N] |
| 32 | 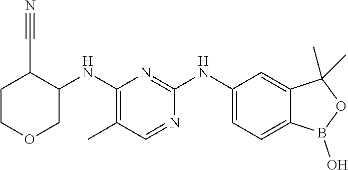 [SMILES: Cc1cnc(Nc2ccc3c(c2)C(C)(C)OB3O)nc1NC1COCCC1C#N] |
| 33 | 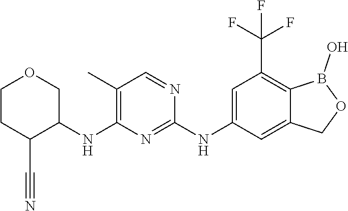 [SMILES: Cc1cnc(Nc2cc3c(c(C(F)(F)F)c2)B(O)OC3)nc1NC1COCCC1C#N] |
| 34 | 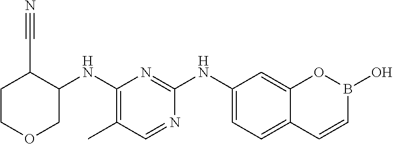 [SMILES: Cc1cnc(Nc2ccc3c(c2)OB(O)C=C3)nc1NC1COCCC1C#N] |
| 35 | 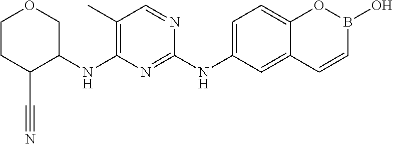 [SMILES: Cc1cnc(Nc2ccc3c(c2)C=CB(O)O3)nc1NC1COCCC1C#N] |
| 36 | 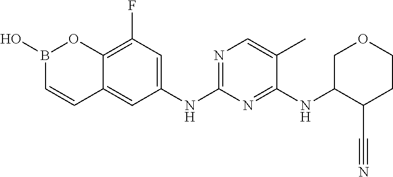 [SMILES: Cc1cnc(Nc2cc(F)c3c(c2)C=CB(O)O3)nc1NC1COCCC1C#N] |
| 37 | 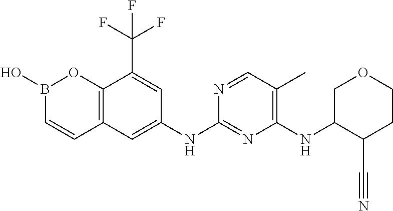 [SMILES: Cc1cnc(Nc2cc3c(c(C(F)(F)F)c2)OB(O)C=C3)nc1NC1COCCC1C#N] |
| 38 | 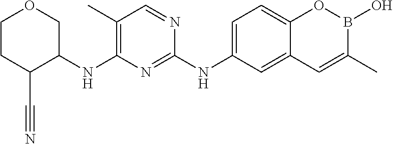 [SMILES: CC1=Cc2cc(Nc3ncc(C)c(NC4COCCC4C#N)n3)ccc2OB1O] |
| 39 | 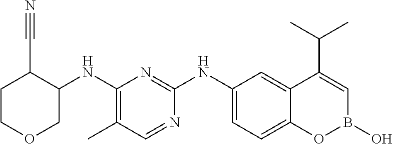 [SMILES: Cc1cnc(Nc2ccc3c(c2)C(C(C)C)=CB(O)O3)nc1NC1COCCC1C#N] |
| 40 | 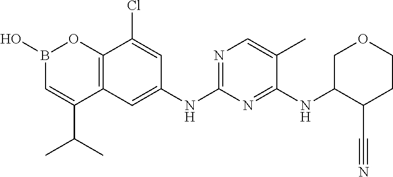 [SMILES: Cc1cnc(Nc2cc(Cl)c3c(c2)C(C(C)C)=CB(O)O3)nc1NC1COCCC1C#N] |
| 41 | 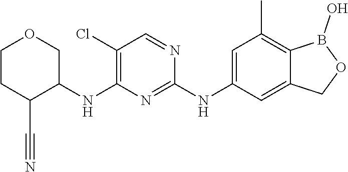 [SMILES: Cc1cc(Nc2ncc(Cl)c(NC3COCCC3C#N)n2)cc2c1B(O)OC2] |
| 42 | 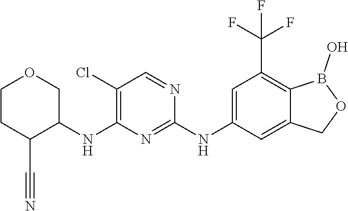 [SMILES: N#CC1CCOCC1Nc1nc(Nc2cc3c(c(C(F)(F)F)c2)B(O)OC3)ncc1Cl] |
| 43 | 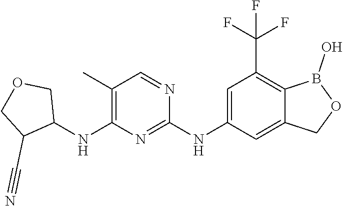 [SMILES: Cc1cnc(Nc2cc3c(c(C(F)(F)F)c2)B(O)OC3)nc1NC1COCC1C#N] |
| 44 | 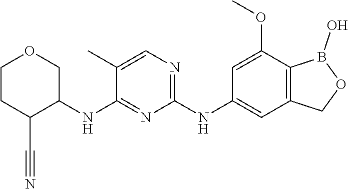 [SMILES: COc1cc(Nc2ncc(C)c(NC3COCCC3C#N)n2)cc2c1B(O)OC2] |
| 45 | 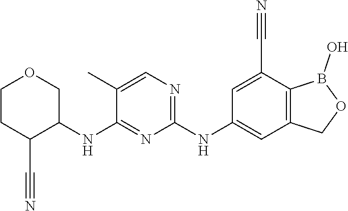 [SMILES: Cc1cnc(Nc2cc(C#N)c3c(c2)COB3O)nc1NC1COCCC1C#N] |
| 46 | 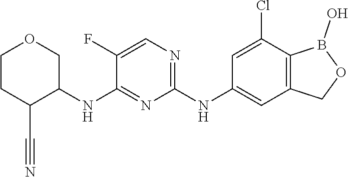 [SMILES: N#CC1CCOCC1Nc1nc(Nc2cc(Cl)c3c(c2)COB3O)ncc1F] |
| 47 | 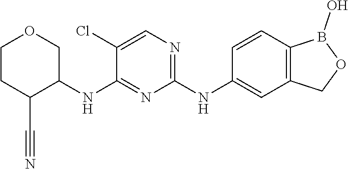 [SMILES: N#CC1CCOCC1Nc1nc(Nc2ccc3c(c2)COB3O)ncc1Cl] |
| 48 | 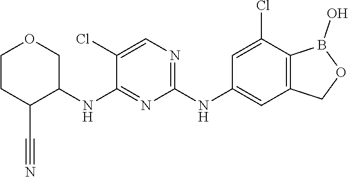 [SMILES: N#CC1CCOCC1Nc1nc(Nc2cc(Cl)c3c(c2)COB3O)ncc1Cl] |
| 49 | 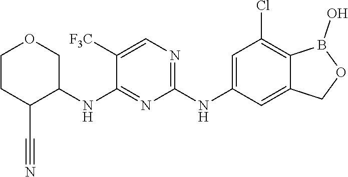 [SMILES: N#CC1CCOCC1Nc1nc(Nc2cc(Cl)c3c(c2)COB3O)ncc1C(F)(F)F] |
| 50 | 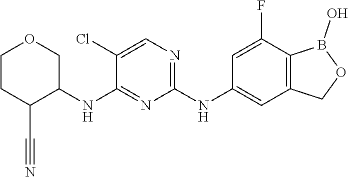 [SMILES: N#CC1CCOCC1Nc1nc(Nc2cc(F)c3c(c2)COB3O)ncc1Cl] |

In one embodiment, the present disclosure provides a compound selected from the group consisting of Examples 1-24 and Examples 25-50 (Table 1 and Table 2) and the compound has trans relative stereochemistry as represented in formula (Ia) and (Ib):

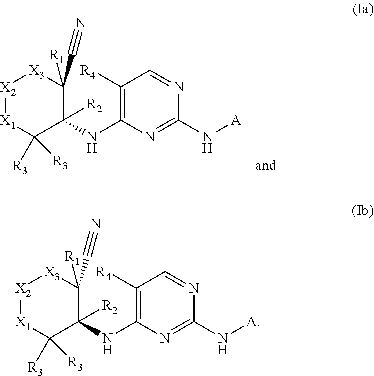

In one embodiment, the present disclosure provides a compound selected from the group consisting of Examples 1-25 (as shown in Table 1) and Examples 26-50 (as shown in Table 2) and has trans relative stereochemistry as represented in formula (Ia).

In one embodiment, the present disclosure provides a compound selected from the group consisting of Examples 1-25 (as shown in Table 1) and Examples 26-50 (as shown in Table 2) and has trans relative stereochemistry as represented in formula (Ib).

### Definitions

Terms used herein will have their customary meaning in the art unless specified otherwise. The organic moieties mentioned in the definitions of the variables of the compound of formula (I), (Ia), and (Ib) are—like the term halogen—collective terms for individual listings of the individual group members. The prefix Cn-Cm (or Cn-m) indicates, in each case, the possible number of carbon atoms in the group.

The term “animal” as used herein includes all mammals, birds and fish and also includes all vertebrate animals. Animals include, but are not limited to, cats, dogs, cattle, chickens, cows, deer, goats, horses, llamas, pigs, sheep and yaks. It also includes an individual animal in all stages of development, including embryonic and fetal stages. In some embodiments, the animal will be a non-human animal.

By the term “enriched” is meant when the weight:weight ratio is at least approximately 1.05 or higher in favor of the enantiomer that displays significant in vitro and in vivo activity (the eutomer).

In one embodiment, the compounds of the instant disclosure are selective JAK1 inhibitors relative to JAK2. The determination of relative selectivity for a given compound of JAK1 inhibition is defined as the relative ratio of the (JAK2 IC₅₀ value/JAK1 IC₅₀ value). In one embodiment, for a given compound, the relative ratio of the (JAK2 IC₅₀ value/JAK1 IC₅₀ value) is at least 2. In yet another embodiment, for a given compound, the relative ratios of the JAK2 IC₅₀ value/JAK1 IC₅₀ value) is at least 5, in another embodiment, the relative ratios of the JAK2 IC₅₀/JAK1 IC₅₀ are preferably at least 10. In one embodiment the ratios of the JAK2 IC₅₀/JAK1 IC₅₀ are greater than 10.

The term “treatment” or “treating” includes alleviating, ameliorating, relieving or otherwise reducing the signs and symptoms associated with a disease or disorder.

“Therapeutically effective amount” means that amount of a drug or pharmaceutical agent that will elicit the biological or medical response of a tissue, a system, animal or human that is being sought by a researcher, veterinarian, medical doctor or other clinician.

The term “composition”, as in pharmaceutical composition, is intended to encompass a product including the active ingredient(s), and the inert ingredient(s)

(pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of formula (I), (Ia), and (Ib) or a mixture thereof, and pharmaceutically acceptable excipients.

The term “optionally substituted” means “unsubstituted or substituted,” and therefore, the generic structural formulas described herein encompasses compounds containing the specified optional substituent as well as compounds that do not contain the optional substituent.

When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

As used herein, “alkyl” refers to monovalent saturated aliphatic hydrocarbon groups having from 1 to 20 carbon atoms, preferably 1-8 carbon atoms, preferably 1-6 carbon atoms. The hydrocarbon chain may be either straight-chained or branched. Illustrative alkyl groups include methyl, ethyl, n-propyl, iso-propyl, n-butyl, iso-butyl, and tert-butyl. Similarly, an “alkenyl” group refers to an alkyl group having one or more double bonds present in the chain, and an “alkynyl” group refers to an alkyl group having one or more triple bonds present in the chain.

As used herein “halogen” or “halo” refers to a halogen. In some embodiments, the halogen is preferably Br, Cl, or F.

As used herein, “haloalkyl” refers to monovalent saturated aliphatic hydrocarbon groups having from 1 to 20 carbon atoms, preferably 1-8 carbon atoms, preferably 1-6 carbon atoms, wherein at least one hydrogen atom is substituted by a halogen, including but not limited to perhalo groups where all hydrogen atoms are replaced with halogen atoms. The haloalkyl chain can be either straight-chained or branched. Illustrative alkyl groups include trifluoromethyl, trifluoroethyl, trifluoropropyl, trifluorobutyl, and pentafluoroethyl. Similarly, a “haloalkenyl” group refers to a haloalkyl group having one or more double bonds present in the chain, and a “haloalkynyl” group refers to a haloalkyl group having one or more triple bonds present in the chain.

Moreover, an “alkylene” linker group refers to a divalent alkyl group, namely (CH₂)ₓ, where x is 1 to 20, preferably 1 to 8, preferably 1 to 6, and more preferably 1 to 3.

As used herein, “hydroxyalkyl” refers to an alkyl group as herein defined substituted with one or more —OH group. Similarly, a “hydroxyalkenyl” group refers to a haloalkyl group having one or more double bonds present in the chain, and a “hydroxyalkynyl” group refers to a haloalkyl group having one or more triple bonds present in the chain.

As used herein, “aryl” refers to a substituted or unsubstituted carbocyclic aromatic ring system, either pendent or fused, such as phenyl, naphthyl, anthracenyl, phenanthryl, tetrahydronaphthyl, indane, or biphenyl. A preferred aryl group is phenyl.

As used herein, “cycloalkyl” refers to an unsaturated or partially saturated hydrocarbon ring, containing from 3 to 15 ring atoms. Illustrative cycloalkyl groups include cyclopropyl, cyclobutyl, cyclopentyl, cyclohexyl, as well as partially saturated versions thereof, such as cyclohexenyl, and cyclohexadienyl. Moreover, bridged rings, such as adamantane, are included within the definition of “cycloalkyl.”

As used herein, the term “heterocyclyl” refers to an unsaturated or partially saturated hydrocarbon ring, containing from 3 to 15 ring atoms, wherein one or more carbon atom is replaced with a heteroatom selected from O, N, S, or Si, where each N, S, or Si may be oxidized, and where each N may be quarternized. A heterocyclyl group may be attached to the remainder of the molecule through a heteroatom. Heterocyclyl does not include heteroaryl.

As used herein, the term “heteroaryl” or “heteroaromatic” refers to aromatic ring groups having 5 to 14 ring atoms selected from carbon and at least one (typically 1-4, more typically 1 or 2) heteroatom (e.g., oxygen, nitrogen, sulfur, or silicon). They include monocyclic rings and polycyclic rings in which a monocyclic heteroaromatic ring is fused to one or more other carbocyclic aromatic or heteroaromatic rings. Examples of monocyclic heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), tetrazolyl (e.g., tetrazolyl) and thienyl (e.g., 2-thienyl, 3-thienyl. Examples of monocyclic six-membered nitrogen-containing heteroaryl groups include pyrimidinyl, pyridinyl and pyridazinyl. Examples of polycyclic aromatic heteroaryl groups include carbazolyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, acridinyl, or benzisoxazolyl.

The terms “arylalkyl,” “heteroarylalkyl,” and “heterocyclylalkyl” refers to those radicals in which an aryl, heteroaryl, or heterocyclyl group is linked through an alkyl group. Examples includes benzyl, phenethyl, pyridylmethyl, and the like. The terms also include alkyl linking groups in which a carbon atom, for example, a methylene group, has been replaced by, for example, an oxygen atom. Examples include phenoxymethyl, pyrid-2-yloxymethyl, 3-(naphth-1-yloxy)propyl, and the like. Similarly, the term “benzyl” as used herein is a radical in which a phenyl group is attached to a CH2 group, thus, a CH2Ph group. Benzyl groups may be substituted or unsubstituted. The term substituted benzyl refers to radicals in which the phenyl group or CH2 contains one or more substituents. In one embodiment, the phenyl group may have 1 to 5 substituents, or in another embodiment 2 to 3 substituents.

As used herein “optionally substituted” refers to a substitution of a hydrogen atom, which would otherwise be present for the substituent. When discussing ring systems, the optional substitution is typically with 1, 2, or 3 substituents replacing the normally-present hydrogen. When referencing straight and branched moieties, however, the number of substitutions may be more, occurring wherever hydrogen is present. The substitutions may be the same or different.

Illustrative substituents, which with multiple substituents can be the same or different, include halogen, haloalkyl, R′, OR′, OH, SH, SR′, NO2, CN, C(O)R′, C(O)(alkyl substituted with one or more of halogen, haloalkyl, NH2, OH, SH, CN, and NO2), C(O)OR′, OC(O)R′, CON(R′)2, OC(O)N(R′)2, NH2, NHR′, N(R′)2, NHCOR′, NHCOH, NHCONH2, NHCONHR′, NHCON(R′)2, NRCOR′, NRCOH, NHCO2H, NHCO2R′, NHC(S)NH2, NHC(S)NHR′, NHC(S)N(R′)2, CO2R′, CO2H, CHO, CONH2, CONHR′, CON(R′)2, S(O)2H, S(O)2R′, SO2NH2, S(O)H, S(O)R′, SO2NHR′, SO2N(R′)2, NHS(O)2H, NR′S(O)2H, NHS(O)2R′, NR′S(O)2R′, Si(R′)3, where each of the preceding may be linked through a divalent alkylene linker, (CH2)x, where x is 1, 2, or 3. In embodiments where a saturated carbon atom is optionally substituted with one or more substituent groups, the substituents may be the same or different and also include ═O, ═S, ═NNHR′, ═NNH2, ═NN(R′)2, ═N—OR′, ═N—OH, ═NNHCOR′, ═NNHCOH, ═NNHCO2R′, ═NNHCO2H, ═NNHSO2R′, ═NNHSO2H, ═N—CN, ═NH, or ═NR′. For each of the preceding, each may be linked through an alkylene linker, (CH2)x, where x is 1, 2, or 3, Each occurrence of R′ is the same or different and, in some embodiments, represents hydrogen, alkyl, alkenyl, alkynyl, cycloalkyl, aryl, heterocyclyl, or heteroaryl, or, in some embodiments, when two R′ are each attached to a nitrogen atom, they may form a saturated or unsaturated heterocyclic ring containing from 4 to 6 ring atoms.

As used herein, the phrase veterinary or veterinarily, or pharmaceutical or pharmaceutically acceptable salt refers to any salt of a compound disclosed herein which retains its biological properties and which is not toxic or otherwise undesirable for veterinary or pharmaceutical use. The general use of the terms pharmaceutical or pharmaceutically is intended to reach either veterinary or veterinarily, as well. The terms may be used interchangeably as context allows.

Such salts may be derived from a variety of organic and inorganic counter-ions known in the art. Such salts include acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and like acids.

Salts further include, by way of example only, salts of non-toxic organic or inorganic acids, such as halides, such as, chloride and bromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

Examples of inorganic bases that may be used to form base addition salts include, but are not limited to, metal hydroxides, such as lithium hydroxide, sodium hydroxide, and potassium hydroxide; metal amides, such as lithium amide and sodium amide; metal carbonates, such as lithium carbonate, sodium carbonate, and potassium carbonate; and ammonium bases such as ammonium hydroxide and ammonium carbonate.

Examples of organic bases that may be used to form base addition salts include, but are not limited to, metal alkoxides, such as lithium, sodium, and potassium alkoxides including lithium methoxide, sodium methoxide, potassium methoxide, lithium ethoxide, sodium ethoxide, potassium ethoxide, and potassium tert-butoxide; quaternary ammonium hydroxides, such as choline hydroxide; and amines including, but not limited to, aliphatic amines (i.e., alkylamines, alkenylamines, alkynylamines, and alicyclic amines), heterocyclic amines, arylamines, heteroarylamines, basic amino acids, amino sugars, and polyamines.

The base may be a quaternary ammonium hydroxide, wherein one or more of the alkyl groups of the quaternary ammonium ion are optionally substituted with one or more suitable substituents. Preferably, at least one alkyl group is substituted with one or more hydroxyl groups. Non-limiting examples of quaternary ammonium hydroxides that may be used in accordance with the present disclosure include choline hydroxide, trimethylethylammonium hydroxide, tetramethylammonium hydroxide, and is preferably choline hydroxide. An alkylamine base may be substituted or unsubstituted. Non-limiting examples of unsubstituted alkylamine bases that may be used in accordance with the present disclosure include methylamine, ethylamine, diethylamine, and triethylamine. A substituted alkylamine base may be substituted with one or more hydroxyl groups, and preferably one to three hydroxyl groups. Non-limiting examples of substituted alkylamine bases that may be used in accordance with the present disclosure include 2-(diethylamino)ethanol, N,N-dimethylethanolamine (deanol), tromethamine, ethanolamine, and diolamine.

### Stereoisomers and Polymorphic Forms

In certain cases, the depicted substituents may contribute to optical isomers and/or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. When a compound has an asymmetric center, for example when it is bonded to four different groups, a pair of enantiomers is possible. A molecule with at least one stereocenter may be characterized by the absolute configuration of its asymmetric center and is designated (R) or (S) according to the rules of Cahn and Prelog (Cahn et al., 1966, Angew. Chem. 78: 413-447, Angew. Chem., Int. Ed. Engl. 5: 385-414 (errata: Angew. Chem., Int. Ed. Engl. 5:511); Prelog and Helmchen, 1982, Angew. Chem. 94: 614-631, Angew. Chem. Internat. Ed. Eng. 21: 567-583; Mata and Lobo, 1993, Tetrahedron: Asymmetry 4: 657-668) or may be characterized by the manner in which the molecule rotates the plane of polarized light and is designated dextrorotatory or levorotatory (namely, as (+)- or (−)-isomers, respectively). A chiral compound may exist as either an individual enantiomer or as a mixture thereof. A mixture containing equal proportions of enantiomers is called a “racemic mixture”.

In certain embodiments, the compounds disclosed herein may possess one or more asymmetric centers, and such compounds may therefore be produced as a racemic mixture, an enantiomerically enriched mixture, or as an individual enantiomer. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art.

In certain embodiments, the compounds disclosed herein are “stereochemically pure”. A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

In addition, the compounds disclosed herein (‘active agents’) may exist as hydrates or solvates, in which a certain stoichiometric amount of water or a solvent is associated with the molecule in the crystalline form. The compositions of the disclosure may include hydrates and solvates of the active agents. In some embodiments, the compositions of the disclosure may include up to 15% (w/w), up to 20% (w/w), or up to 30% (w/w) of a particular solid form.

As used herein, the terms “subject” and “patient” may be used interchangeably herein. In one embodiment, the subject is a human. In one embodiment, the subject is a companion animal such as a dog or cat. In a further embodiment, the subject is an animal such as a sheep, cow, horse, goat, fish, pig, or domestic fowl (e.g., chicken, turkey, duck, or goose). In another embodiment, the subject is a primate such as a monkey such as a cynomolgous monkey or a chimpanzee.

In addition, a pharmaceutically acceptable prodrug of the compound represented by the formula (I), formula (Ia), and formula (Ib) is also included in the present disclosure. The pharmaceutically acceptable prodrug refers to a compound having a group which may be converted into an amino group, a hydroxyl group, a carboxyl group, or the like, by solvolysis or under a physiological condition. Examples of the groups forming the prodrug include those as described in Prog. Med., 5, 2157-2161 (1985) or “Pharmaceutical Research and Development” (Hirokawa Publishing Company, 1990), vol. 7, Drug Design, 163-198. The term prodrug is used throughout the specification to describe any pharmaceutically acceptable form of a compound which, upon administration to a patient, provides the active compound. Pharmaceutically acceptable prodrugs refer to a compound that is metabolized, for example hydrolyzed or oxidized, in the host to form the compound of the present disclosure. Typical examples of prodrugs include compounds that have biologically labile protecting groups on a functional moiety of the active compound. Prodrugs include compounds that may be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound.

The present disclosure includes all pharmaceutically acceptable isotopically-labelled compounds wherein one or more atoms are replaced by atoms having the same atomic number, but an atomic mass or mass number different from the atomic mass or mass number usually found in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of the disclosure, such as those incorporating a radioactive isotope, may be useful in drug or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds of the disclosure may generally be prepared by conventional techniques known to those skilled in the art or by processes analogous to those described in the accompanying Examples using an appropriate isotopically-labeled reagent in place of the non-labeled reagent previously employed. In one aspect, the disclosure provides a process for preparing a compound of any embodiment of formula (I), formula (Ia) and formula (Ib) as described herein.

### Compositions and Methods of Administration

The compounds of formula (I), formula (Ia) and formula (Ib) used in the methods disclosed herein may be administered in certain embodiments using veterinary or pharmaceutical compositions including at least one compound of formula (I), formula (Ia) and formula (Ib), if appropriate in the salt form, either used alone or in the form of a combination with one or more compatible and veterinary or pharmaceutically acceptable carriers, such as diluents or adjuvants, or with another agent. There are provided compositions which comprise a derivative of a compound of formula (I), formula (Ia) and formula (Ib) or a salt thereof, and an acceptable excipient, carrier or diluent. The composition may also be in a variety of forms which include, but are not limited to, oral formulations, injectable formulations, and topical, dermal or subdermal formulations. The particular route selected by the practitioner depends upon factors such as the physicochemical properties of the pharmaceutical or therapeutic agent, the condition of the host and economics.

In one aspect, the disclosure provides for a method for treating a patient having a disease or disorder susceptible to modulation of JAK including administering a therapeutically effective amount of a compound as described herein, including any embodiment of a compound according formula (I), (Ia), or (Ib).

In one embodiment, the disclosure provides a method of treating a patient having a disease or disorder that can be ameliorated by the selective inhibition of a Janus kinase JAK 1 relative to JAK 2.

In one embodiment, the disclosure provides for a method of treating a patient having a disease or disorder with a therapeutically effective amount of a compound which is an embodiment of formula (I), formula (Ia) and formula (Ib), wherein the disease or disorder is one or more of atopic dermatitis, flea allergy dermatitis, eczema, pruritus, psoriasis, psoriatic arthritis, Bechet's disease, pityriasis rubra pilaris, alopecia areata, discoid lupus erythematosus, vitiligo, palmoplantar pustulosis, mucocutaneous disease erythema multiforme, mycosis fungoides, graft-versus-host disease, cutaneous lupus, rheumatoid arthritis (RA), arthritis, ulcerative colitis, Crohn's disease, inflammatory bowel disease (IBD), transplant rejection, systemic lupus erythematosus (SLE), dermatomyositis, Sjogren's syndrome, dry eye disease, secondary hypereosinophilic syndrome (HES), allergy, allergic dermatitis, asthma, vasculitis, multiple sclerosis, diabetic nephropathy, cardiovascular disease, artherosclerosis, and cancer.

In one embodiment, the disclosure provides a method of treating a patient as described above, wherein the the disease or disorder is one or more of atopic dermatitis, flea allergy dermatitis psoriasis, and rheumatoid arthritis.

According to one embodiment, the disclosure provides for a method of treating a patient having a disease or disorder susceptible to modulating of JAK and including administering an amount of a compound according to the disclosure in an amount to perturb an immune regulatory pathway in a cell. In one such embodiment, the perturbation results in an effect on the JAK-STAT pathway.

In one aspect, the disclosure provides a method of inhibiting JAK in a mammalian cell including contacting the mammalian cell with a compound according to any embodiment of formula (I), (Ia), or (Ib). In certain embodiments, the mammalian cell is a cell from a subject having an inflammatory condition.

In one aspect, the disclosure provides for a composition including a compound according to any embodiment of formula (I), formula (Ia) and formula (Ib) and a pharmaceutically or veterinary acceptable carrier.

The composition may be in a form suitable for oral use, for example, as dietary supplements, troches, lozenges, chewables, tablets, hard or soft capsules, emulsions, aqueous or oily suspensions, aqueous or oily solutions, dispersible powders or granules, syrups, or elixirs. Compositions intended for oral use may be prepared according to any method known in the art for the manufacture of veterinary or pharmaceutical compositions and such compositions may contain one or more agents selected from the group consisting of sweetening agents, bittering agents, flavoring agents, coloring agents and preserving agents in order to provide elegant and palatable preparations. In certain cases, it is convenient and efficient to administer veterinary medicines orally by placing the therapeutic agent in a solid or liquid matrix that is suitable for oral delivery. These methods include chewable drug-delivery formulations. The problem associated with administering oral formulations to animals is that the therapeutic agent often provides an unpleasant taste, aroma, or texture, which causes the animals to reject the composition. This is further exacerbated by compositions that are hard and difficult to swallow.

Oral veterinary compositions in the form of soft chewable compositions (“soft chews”), or chewable tablets that are palatable are usually convenient to administer to certain animals, particularly cats and dogs, and may be used effectively to dose veterinary medicine to these animals. However, many oral compositions including active agents with a bitter or unpleasant taste are not well accepted by cats and dogs. Furthermore, when the bioavailability of an active agent from an oral dosage form is not sufficient or is variable, the required exposure of the animal to the active ingredient may not be sufficient to provide the desired efficacy. Problems such as these often lead to low or sub-optimal efficacy and control of parasites.

Chewable dosage forms for drug delivery are well known to pharmaceutical technology. It is known in the pharmaceutical industry that the act of chewing increases the surface area of the available active ingredient and may increase the rate of absorption by the digestive tract. Chewable systems are also advantageous where it is desirable to make an active ingredient available topically to the mouth or throat areas for both local effects and/or systemic absorption. Further, chewable dosage forms are also utilized to ease drug administration in pediatric and geriatric patients. Examples of chewable dosage forms may be found in U.S. Pat. Nos. 6,387,381; 4,284,652; 4,327,076; 4,935,243; 6,270,790; 6,060,078; 4,609,543; and 5,753,255, all incorporated herein by reference.

Palatability and “mouth feel” are important characteristics to be considered in providing a dosage form, or matrix, for an active pharmaceutical or medicinal. Unfortunately, many pharmaceuticals and other active ingredients have a bitter or otherwise unpalatable taste, or an unacceptable mouth feel, due to the grittiness or chalkiness of the compound, or both. These characteristics make it difficult to incorporate such active ingredients into the current state of the art for chewable dosage forms because the objectionable taste and/or mouth feel make it less likely to obtain compliance by the user. Oral veterinary dosage forms that are not palatable to the animal treated result in low acceptance of the medicament by the animal and a low level of compliance. Thus, there is a need for improved oral veterinary dosage forms that are palatable and well accepted by the treated animal.

Another challenge with oral veterinary compositions, particularly soft chewable compositions, is that the release and dissolution of the active agent from the composition after it is ingested by the animal can be variable and incomplete. This leads to variability in the amount of the drug that is absorbed from the digestive tract of the animal.

U.S. Pat. No. 7,955,632 (incorporated herein by reference) describes palatable, edible soft chewable medication vehicles for the delivery of pharmaceutically acceptable active ingredients to an animal and processes of making the same.

Furthermore, US 2004/0037869 A1, US 2004/0151759 A1, WO 2005/062782 and WO 2004/016252 to Cleverly et al. (incorporated herein by reference) describe chewable veterinary formulations and tablets that contain at least one pharmaceutical active agent, and WO 2009/02451A2 and US 2011/0059988 to Heckeroth et al. describe various compositions for oral administration to animals (all incorporated herein by reference).

Traditionally, in veterinary formulations, palatability had been achieved by the inclusion of animal byproducts or flavors derived from animal sources into the formulation. For example, it is customary to include excipients, such as chicken powder, liver powder, beef, ham, fish, or rawhide-derived products in dog chews to make the chew attractive and palatable to the dog. See, e.g., U.S. Pat. Nos. 6,086,940; 6,093,441; 6,159,516; 6,110,521; 5,827,565; 6,093,427, all to Axelrod et at. (all incorporated herein by reference).

Exceptionally palatable soft chewable oral veterinary compositions that provide high bioavailability of active agent are described in U.S. Pat. Nos. 9,259,417; 9,233,100; 9,931,320; 10,596,156; all to Soll et al. (all incorporated herein by reference).

Lozenges are solid compositions containing one or more active ingredients intended to dissolve or disintegrate slowly in the oral cavity by passive incubation in the oral cavity, or actively by sucking or chewing. They may be used for systemic effect if the drug is absorbed through the buccal or esophageal lining or is swallowed. In particular, soft lozenges may be chewed or allowed to dissolve slowly in the mouth. These dosage forms have the advantage of being flavored and thus easy to administer to both human and animal patients; have formulas that are easy to change and may be patient specific; may deliver accurate amounts of the active ingredient to the oral cavity and digestive system; and allow for the drug to remain in contact with the oral or esophageal cavity for an extended period of time.

Tablets may contain the active ingredient in admixture with non-toxic, pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period.

Formulations for oral use may be hard gelatin capsules, wherein the active ingredient is mixed with an inert solid diluent, for example, calcium carbonate, calcium phosphate or kaolin. Capsules may also be soft gelatin capsules, wherein the active ingredient is mixed with water or miscible solvents such as propylene glycol, PEGs and ethanol, or an oil medium, for example, peanut oil, liquid paraffin, or olive oil.

The compositions may also be in the form of oil-in-water or water-in-oil emulsions. The oily phase may be a vegetable oil, for example, olive oil or arachis oil, or a mineral oil, for example, liquid paraffin or mixtures of these. Suitable emulsifying agents may be naturally-occurring phosphatides, for example, soy bean, lecithin, and esters or partial esters derived from fatty acids and hexitol anhydrides, for example, sorbitan monoleate, and condensation products of the said partial esters with ethylene oxide, for example, polyoxyethylene sorbitan monooleate. The emulsions may also contain sweetening agents, bittering agents, flavoring agents, and preservatives.

In one embodiment of the formulation, the composition is in the form of a microemulsion. Microemulsions are well suited as the liquid carrier vehicle. Microemulsions are quaternary systems including an aqueous phase, an oily phase, a surfactant and a cosurfactant. They are translucent and isotropic liquids. Microemulsions are composed of stable dispersions of microdroplets of the aqueous phase in the oily phase or conversely of microdroplets of the oily phase in the aqueous phase. The size of these microdroplets is less than 200 nm (1000 to 100,000 nm for emulsions). The interfacial film is composed of an alternation of surface-active (SA) and co-surface-active (Co-SA) molecules which, by lowering the interfacial tension, allows the microemulsion to be formed spontaneously. In one embodiment of the oily phase, the oily phase may be formed from mineral or vegetable oils, from unsaturated polyglycosylated glycerides or from triglycerides, or alternatively from mixtures of such compounds. In one embodiment of the oily phase, the oily phase comprises of triglycerides; in another embodiment of the oily phase, the triglycerides are medium-chain triglycerides, for example, C₈-C₁₀ caprylic/capric triglyceride. In another embodiment, the oily phase will represent a % v/v range selected from the group consisting of about 2 to about 15%; about 7 to about 10%; and about 8 to about 9% v/v of the microemulsion. The aqueous phase includes, for example, water or glycol derivatives, such as propylene glycol, glycol ethers, polyethylene glycols or glycerol. In one embodiment of the glycol derivatives, the glycol is selected from the group consisting of propylene glycol, diethylene glycol monoethyl ether, dipropylene glycol monoethyl ether and mixtures thereof. Generally, the aqueous phase will represent a proportion from about 1 to about 4% v/v in the microemulsion. Surfactants for the microemulsion include diethylene glycol monoethyl ether, dipropylene glycol monomethyl ether, polyglycolyzed C₈-C₁₀ glycerides or polyglyceryl-6 dioleate. In addition to these surfactants, the cosurfactants include short-chain alcohols, such as ethanol and propanol. Some compounds are common to the three components discussed above, for example, aqueous phase, surfactant and cosurfactant. However, it is well within the skill level of the practitioner to use different compounds for each component of the same formulation. In one embodiment, for example, for the amount of surfactant/cosurfactant, the cosurfactant to surfactant ratio may be from about 1/10 to about 1/2. In another embodiment for the amount of cosurfactant, there will be from about 25 to about 75% v/v of surfactant and from about 10 to about 55% v/v of cosurfactant in the microemulsion.

Oily suspensions may be formulated by suspending the active ingredient in a vegetable oil, for example, atachis oil, olive oil, sesame oil or coconut oil, or in mineral oil such as liquid paraffin. The oily suspensions may contain a thickening agent, for example, beeswax, hard paraffin or cetyl alcohol. Sweetening agents such as sucrose, saccharin or aspartame, bittering agents, and flavoring agents may be added to provide a palatable oral preparation. These compositions may be preserved by the addition of an anti-oxidant such as ascorbic acid, or other known preservatives.

Aqueous suspensions may contain the active material in admixture with excipients suitable for the manufacture of aqueous suspensions. Such excipients are suspending agents, for example, sodium carboxymethylcellulose, methylcellulose, hydroxypropylmethylcellulose, sodium alginate, polvinylpyrrolidone, gum tragacanth and gum acacia; dispersing or wetting agents may be a naturally-occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide, with partial esters derived from fatty acids and hexitol anhydrides, for example, polyethylene sorbitan monooleate. The aqueous suspensions may also contain one or more preservatives, for example, ethyl, or n-propyl, p-hydroxybenzoate, one or more coloring agents, one or more flavoring agents, and one or more sweetening agents and/or bittering agents, such as those set forth herein.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, bittering, flavoring and coloring agents, may also be present.

Syrups and elixirs may be formulated with sweetening agents, for example, glycerol, propylene glycol, sorbitol or sucrose. Such formulations may also contain a demulcent, a preservative, flavoring agent(s) and coloring agent(s).

The compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example, as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. Preservatives, such as phenol or benzyl alcohol, may be used.

In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Topical, dermal and subdermal formulations may include emulsions, creams, ointments, gels or pastes.

Organic solvents that may be used include but are not limited to: acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone (e.g. N-methylpyrrolidone), diethylene glycol monoethyl ether, ethylene glycol and diethyl phthalate, or a mixture of at least two of these solvents.

As vehicle or diluent, compositions of the present disclosure may include plant oils such as, but not limited to soybean oil, groundnut oil, castor oil, corn oil, cotton oil, olive oil, grape seed oil, sunflower oil, etc.; mineral oils such as, but not limited to, petrolatum, paraffin, silicone, etc.; aliphatic or cyclic hydrocarbons or alternatively, for example, medium-chain (such as C₈-C₁₂) triglycerides.

Dosage forms may contain from about 0.5 mg to about 5 g of an active agent.

In one embodiment of the disclosure, the active agent is present in the formulation at a concentration of about 0.05 to 10% weight/volume.

A compound of formula (I), formula (a) and formula (IVb) may be employed as such or in the form of their preparations or formulations as combinations.

A compound of formula (I), formula (Ia) and formula (Ib) according to the disclosure may be combined with one or more agents having the same sphere of activity, for example, to increase activity, or with substances having another sphere of activity, for example, to broaden the range of activity. As an example, a combination of a compound of formula (I) (or of a formula (Ia) and formula (Ib)) with one or more of an additional JAK inhibitor or a JAK/Signal Transducer and Activator of Transcription (JAK/STAT) modulator may offer therapeutic advantage. Examples of JAK inhibitors that may be useful as combination agents include Baricitinib, Ruxolitinib, Filgotinib, CYT387, Upadacitinib, Fedratinib, Peficitinib, Lestaurtinib, Pacritinib, Oclacitinib, Cerdulatinib, and Tofacitinib.

The compounds of formula (I), formula (Ia) or formula (Ib) according to the disclosure may be combined with one or more additional pharmaceutical or veterinary active agents. Further additional active agents which may be used in the methods provided herein in combination with a compound of formula (I), (Ia) or (Ib) include, but are not limited to, disease-modifying anti-rheumatic drugs (DMARDs such as cyclosporine A and methotrexate), anti-inflammatory agents such as nonsteroidal anti-inflammatory drugs (NSAIDs), immnunosuppressants, mycophenolate mofetil, biologic agents, TNF-a inhibitors (such as etanercept), Cox-2 inhibitors (such as firocoxib), and analgesics. These agents may include but are not limited to cyclosporin A, e.g. Sandimmune® or Neoral®, rapamycin, FK-506 (tacrolimus), leflunomide, deoxyspergualin, mycophenolate, e.g., Cellcept®, azathioprine, e.g. Imuran®, daclizumab, e.g. Zenapax®, OKT3, e.g. Orthocolone®, AtGam, aspirin, acetaminophen, ibuprofen, naproxen, piroxicam, and anti-inflammatory steroids, e.g. prednisolone or dexamethasone.

In some embodiments, the second active agents may include, but are not limited to, anti-inflammatories such as NSAIDs including, but not limited to, diclofenac (e.g., ARTHROTEC®), diflunisal (e.g., DOLOBID®), etodolac (e.g., LODINE®), fenoprofen (e.g., NALFON®), ibuprofen (e.g., ADVIL®, CHILDREN'S ADVIL/MOTRIN®, MEDIPREN®, MOTRIN®, NUPRIN®, or PEDIACARE FEVER®), indomethacin (e.g., ARTHREXIN®), ketoprofen (e.g., ORUVAIL®), ketorolac (e.g., TORADOL®), fosfomycin tromethamine (e.g., MONURAL®), meclofenamate (e.g., MECLOMEN®), nabumetone (e.g., RELAFEN®), naproxen (e.g., ANAPROX®, ANAPROX® DS, EC-NAPROSYN®, NAPRELAN® or NAPROSYN®), oxaprozin (e.g., DAY PRO®), piroxicam (e.g., FELDENE®), sulindac (e.g., CLINORIL®), and tolmetin (e.g., TOLECTIN® DS or TOLECTIN®).

In other embodiments, the second active agents may include, but are not limited to, disease-modifying antirheumatic drugs (e.g., DMARDs) or immnunosuppressants such as, but not limited to, methotrexate (e.g., RHEUMATREX®), sulfasalazine (e.g., AZULFIDINE®), and cyclosporine (e.g., SANDIMMUNE® or NEROAL®; and including cyclosporine A).

In other embodiments, the second active agents may include, but are not limited to, mycophenolate mofetil (e.g., CellCept®), an immunosuppressive agent widely used in organ transplantation and gaining favor in treating autoimmune and inflammatory skin disorders.

In further embodiments, the second active agents may include, but are not limited to, biologic agents such as etanercept (e.g., ENBREL®), infliximab (e.g., REMICADE®) and adalimumab (e.g., HUMIRA®).

In further embodiments of interest, the second active agents may include, but are not limited to Cox-2 inhibitors such as celecoxib (e.g., CELEBREX®), firocoxib (e.g. PREVICOX®), valdecoxib (e.g., BEXTRA®) and meloxicam (e.g., MOBIC®, METACAM®).

In further embodiments, the second active agents may include veterinary therapeutic agents that are well-known in the art (see e.g. Plumb's Veterinary Drug Handbook, 5th Edition, ed. Donald C. Plumb, Blackwell Publishing, (2005) or The Merck Veterinary Manual, 9th Edition, (January 2005)) and include but are not limited to acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitriptyline, amlodipine besylate, ammonium chloride, ammonium molybdenate, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, clorsulon, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide, clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cyclosporine, cyproheptadine, cytarabine, dacarbazine, dactinomycin/actinomycin D, dalteparin sodium, danazol, dantrolene sodium, dapsone, decoquinate, deferoxamine mesylate, deracoxib, deslorelin acetate, desmopressin acetate, desoxycorticosterone pivalate, detomidine, dexamethasone, dexpanthenol, dexraazoxane, dextran, diazepam, diazoxide (oral), dichlorphenamide, diclofenac sodium, dicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprozole, ondansetron, opiate antidiarrheals, orbifloxacin, oxacillin sodium, oxazepam, oxibutynin chloride, oxymorphone, oxytretracycline, oxytocin, pamidronate disodium, pancreplipase, pancuronium bromide, paromomycin sulfate, parozetine, pencillamine, general information penicillins, penicillin G, penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenytoin sodium, pheromones, parenteral phosphate, phytonadione/vitamin K-1, pimobendan, piperazine, pirlimycin, piroxicam, polysulfated glycosaminoglycan, ponazuril, potassium chloride, pralidoxime chloride, prazosin, prednisolone/prednisone, primidone, procainamide, procarbazine, prochlorperazine, propantheline bromide, Propionibacterium acnes injection, propofol, propranolol, protamine sulfate, pseudoephedrine, psyllium hydrophilic mucilloid, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/l-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocainide, tolazoline, telfenamic acid, topiramate, tramadol, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, and zonisamide and mixtures thereof.

These one or more additional active agents may be administered as part of the same or separate dosage forms, via the same or different routes of administration, and on the same or different administration schedules according to standard pharmaceutical practice known to one skilled in the art.

The pharmaceutical preparation including the compounds of formula (Ia), (Ib), and (I) for delivery to a human or other mammal, is preferably in unit dosage form, in which the preparation is subdivided into unit doses containing an appropriate quantity of the active component. The unit dosage form may be a packaged preparation containing discrete quantities of the preparation, such as packaged tablets, capsules, and powders in vials or ampoules. Also, the unit dosage form may be a capsule, tablet or lozenge itself, or it may be an appropriate number of any of these in packaged form.

The quantity of active component in a unit dose preparation may be varied or adjusted from about 0.1 mg to about 1000 mg, according to the particular application and the potency of the active component. The composition may, if desired, also contain other compatible therapeutic agents. In therapeutic use for the treatment or alleviation of inflammation, auto-immune diseases, and cancer in a human or other mammal, the compounds utilized in the method of treatment are administered at an initial dosage of about 0.1 mg/kg to about 100 mg/kg per interval, about 0.1 mg/kg to about 50.0 mg/kg per interval, about 0.1 mg/kg to about 10.0 mg/kg per interval, about 0.1 mg/kg to about 5.0 mg/kg per interval, about 0.1 mg/kg to about 2.5 mg/kg per interval, about 0.1 mg/kg to about 2.0 mg/kg per interval, about 0.1 mg/kg to about 1.0 mg/kg per interval, about 0.4 mg/kg to about 1.0 mg/kg per interval, or about 0.4 mg/kg to about 0.6 mg/kg per interval. Preferred intervals may be daily, weekly, monthly, quarterly, semi-annually, or annually.

The dosages may be varied depending on the requirements of the patient, for example, the size of the human or mammal being treated, the severity of the condition being treated, the route of administration, and the potency of the compound(s) being used. Determination of the proper dosage and route of administration for a particular situation is within the skill of the practitioner. Generally, the treatment will be initiated with smaller dosages, which are less than the optimum dose of the compound, which may be increased in small increments until the optimum effect under the particular circumstances of the condition is reached. For convenience, the total daily dosage may be divided and administered in portions during the day if desired.

In therapeutic use, the compounds of formula (Ia), (Ib), and (I) are useful in manufacture of a medicament for a method of the treating any indication where inhibition of JAK would be desirable, including but not limited to cancer, neuroinflammation, inflammatory airway diseases, ankylosing spondylitis, inflammatory bowel diseases, rheumatoid arthritis, psoriasis, and atopic dermatitis. In one or more embodiments, one or more of a compound of formula (Ia), (Ib), and (I) is useful in the treatment of one or more of atopic dermatitis, psoriasis, psoriatic arthritis, Bechet's disease, pityriasis rubra pilaris, alopecia areata, discoid lupus erythematosus, vitiligo, palmoplantar pustulosis, mucocutaneous disease erythema multiforme, mycosis fungoides, graft-versus-host disease, cutaneous lupus, rheumatoid arthritis (RA), arthritis, ulcerative colitis, Crohn's disease, inflammatory bowel disease (IBD), transplant rejection, systemic lupus erythematosus (SLE), dermatomyositis, Sjogren's syndrome, dry eye disease, secondary hypereosinophilic syndrome (HES), allergy, asthma, vasculitis, multiple sclerosis, diabetic nephropathy, cardiovascular disease, artherosclerosis, and cancer. One route of administration may be oral. One route of administration may be topical.

In one aspect, the present disclosure provides for a method of treating one or more diseases or disorders of inflammation, auto-immune dysfunction, and cancer including administering to a subject in need thereof an effective amount of a compound that is an embodiment herein of formula (I), (Ia) or (Ib).

In one embodiment, the present disclosure provides a method of treating atopic dermatitis, flea allergy dermatitis, psoriasis, or rheumatoid arthritis including administering to a subject in need thereof an effective amount of a compound that is an embodiment of formula (I), (Ia) or (Ib). In one embodiment, the compound is administered orally; in one embodiment, the compound is administered parenterally; in one embodiment, the compound is administered topically.

In one embodiment, the present disclosure provides for a method for treating one or more diseases or disorders of inflammation, auto-immune dysfunction, and cancer including administering to a subject in need thereof an effective amount of a compound according to any embodiment herein of formula (I), (Ia) or (Ib) wherein the subject is a mammal.

In one embodiment, the present disclosure provides for method for treating one or more diseases or disorders of inflammation, auto-immune dysfunction, and cancer including administering to a subject in need thereof an effective amount of a compound according to any embodiment herein of formula (I), (Ia) or (Ib) wherein the subject is selected from one or more of livestock mammals, domestic mammals, and companion animals. In one embodiment, the mammal is one or more of humans, cattle, sheep, goats, llamas, alpacas, pigs, horses, donkeys, dogs, and cats. In one embodiment, the mammal is a human, a dog, or a cat.

In one aspect, the present disclosure provides a compound for use in medicine, wherein the compound is an embodiment of formula (I), (Ia), or (Ib) herein. In another aspect, the present disclosure provides for use of a compound according to any embodiment herein of any of formula (I), (Ia), and (Ib) for the manufacture of a medicament for the treatment of one or more diseases or disorder of inflammation, auto-immune dysfunction, and cancer. In one embodiment, the present disclosure provides for use of a compound according to any embodiment herein of formula (I), (Ia), or (Ib) for the manufacture of a medicament for the treatment of a disease or disorder that can be be ameliorated by the selective inhibition of a Janus kinase JAK 1 relative to JAK 2. In one embodiment, the disease or disorder is atopic dermatitis, flea allergy dermatitis, psoriasis, or rheumatoid arthritis. In one aspect, the present disclosure provides for use of a compound according to an embodiment of formula (I), (Ia) or (Ib) herein and a second active agent in the manufacture of a medicament for the treatment of a disease or a disorder that can be ameliorated by the selective inhibition of a Janus kinase JAK 1 relative to JAK 2. In one aspect, the disclosure provides for use of a compound according to an embodiment of formula (I), (Ia), or (Ib) herein for the treatment of one or more diseases or disorders of inflammation, auto-immune dysfunction, and cancer. In one embodiment, the disease or disorder is atopic dermatitis, flea allergy dermatitis, psoriasis, or rheumatoid arthritis.

The present disclosure explicitly encompasses compounds described herein, including salt forms thereof, and salt forms thereof. The present disclosure also encompasses those compounds presented herein, including stereoisomers thereof. The compounds encompassed by the present disclosure include, in some embodiments, compounds selected from Tables 1 and 2 as described herein.

A composition including a therapeutically acceptable amount of any of the compounds described herein is within the scope of the disclosure. The composition may further comprise a pharmaceutically or veterinary acceptable excipient, diluent, carrier, or mixture thereof. Such a composition may be administered to a subject in need thereof to treat or control a disease or disorder mediated, in whole or in part, directly or indirectly, by JAK. The composition may further comprise an additional active agent, as described herein.

### Examples

### Experimental Procedures:

The following examples provide a more detailed description of the process conditions for preparing compounds of the present disclosure. It is to be understood, however, that the disclosure, as fully described herein and as recited in the claims, is not intended to be limited by the details of the following schemes or modes of preparation.

Certain abbreviations may be used in describing the examples of the present disclosure. The abbreviations are believed to be used consistently within commonly accepted use of those skilled in the art.

### Chemistry Examples

In the following schemes, general substituent groups are represented with assignments that may not align with the formulae of the present disclosure. The following schemes provide a key for such substituent groups that should be followed for the schemes and not applied to the formulae of the present disclosure.

The following abbreviations are used in the chemistry example schemes herein.

### Abbreviations

| | |
|---|---|
| ACN | acetonitrile |
| Ag₂SO₄ | Silver sulfate |
| Aq. | aqueous |
| Boc₂O | Di-tert-butyl dicarbonate |
| ° C. | degree celsius |
| conc. | concentrated |
| DCC | N,N′-dicyclohexylmethanediimine |
| DCM | dichloro methane |
| 1,2-DCE | 1,2-dichloro ethane |
| DEE | diethyl ether |
| DIPE | diisopropylether |
| DIBAL | Diisobutylaluminum hydride |
| DIEA | N,N-diethyl amine |
| DIPEA | N,N-diisopropylethylamine |
| DMA | Dimethylacetamide |
| DMAP | Dimethylaminopyridine |
| 1,2-DME | 1,2-dimethyl ether |
| DMF | N,N-dimethylformamide |
| DMSO | dimethyl sulfoxide |
| ESI-MS | electrospray ionisation mass spectrometry |
| Et₂O | diethylether |
| EtOAc/EE/EA | ethyl acetate |
| eq | equivalent |
| FA | Formic Acid |
| h | hour |
| HPLC | high performance liquid chromatography |
| HOAc | acetic acid |
| HCl | hydrochlorid acid |
| H₂0 | water |
| IPA | Isopropyl alcohol |
| LiBH₄ | Lithium borohydride |
| LiOH | lithium hydroxide |
| LCMS | Liquid chromatography mass spectrometry |
| KOAc | Potassium acetate |
| MeOH | methanol |
| MeCN | acetonitrile |
| NaHCO₃ | sodium bicarbonate |
| NaBH₄ | Sodium borohydride |
| Na₂SO₄ | sodium sulfate |
| NBS | N-bromosuccinimide |
| NClS | N-chlorosuccinimide |
| NH₃ | ammonia |
| NH₃H₂O | Aqueous ammonia |
| NH₄Cl | ammonium chloride |
| NIS | N-iodosuccinimide |
| NMP | N-methyl-2-pyrrolidone |
| Mg | Magnesium turnings |
| min | minute |
| mL | milliliter |
| MTBE | Methyl tert-butyl ether |
| PE | petroleum ether |
| RT | room temperature (about 20° C.) |
| Rₜ | retention time |
| sat. | saturated |
| SFC | Supercritical fluid chromatography |
| SiO₂ | Silicon Dioxide |
| TBAF | Tetrabutylammonium fluoride |
| TBSOTf | tert-Butyldimethylsilyl trifluoromethanesulfonate |
| TEA | triethylamine |
| TFA | trifluoroacetic acid |
| TsOH | Tosic acid |
| THF | tetrahydrofuran |
| TLC | thin-layer chromatography on SiO₂ |
| TMSCN | Trimethylsilylcyanide |
| Zn(CN)₂ | Zinc cyanide |

### Common Intermediate Syntheses

### Preparation of (trans)-2-[(2-chloro-5-methyl-pyrimidin-4-yl)amino]cyclopentanecarbonitrile

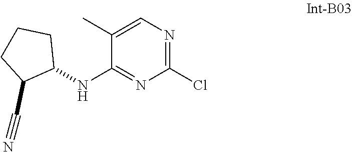

### Preparation of 6-(p-tolylsulfonyl)-6-azabicyclo[3.1.0]hexane

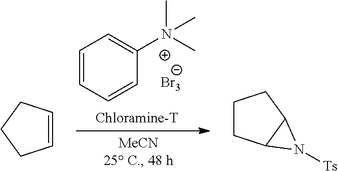

Two reaction mixtures were carried out in parallel. To a mixture of cyclopentene (50 g, 734 mmol, 64.9 mL, 1 eq) and [chloro(p-tolylsulfonyl)amino]sodium (184 g, 807 mmol, 1.1 eq) in MeCN (2.5 L) was added Phenyltrimethylammonium tribromide (27.6 g, 73.4 mmol, 0.1 eq) in portions at 25° C. under N₂. The mixture was stirred at 25° C. for 48 h. TLC showed the reaction was complete. The combined reactions were combined and concentrated under vacuum to give a residue. The residue was quenched with H₂O (2 L) and extracted with EtOAc (1 L×2). The combined organic phases were washed with brine (1 L), dried over Na₂SO₄, filtered, and concentrated under reduced pressure to give a residue. The residue was purified by column chromatography (SiO₂, Petroleum ether/ethyl acetate=5/1 to 4/1) to give 6-(p-tolylsulfonyl)-6-azabicyclo[3.1.0]hexane (125 g, 35.8% yield) as a white solid. ¹H NMR (CDCl₃, 400 MHz): 1.36-1.45 (m, 1H) 1.57-1.69 (m, 3H) 1.89-2.00 (m, 2H) 2.44 (s, 3H) 3.33 (s, 2H) 7.29-7.38 (m, 2H) 7.81 (d, J=8.25 Hz, 2H).

### Preparation of N-(trans)-2-cyanocyclopentyl)-4-methylbenzenesulfonamide

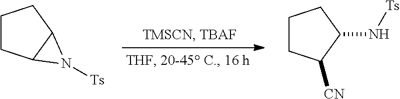 [SMILES: Cc1ccc(S(=O)(=O)N2C3CCCC32)cc1 ; Cc1ccc(S(=O)(=O)N[C@H]2CCC[C@@H]2C#N)cc1]

Two reactions were carried out in parallel. For each a solution of 6-(p-tolylsulfonyl)-6-azabicyclo[3.1.0]hexane (55 g, 231 mmol, 1 eq) in THF (550 mL) was added TMSCN (34.4 g, 347 mmol, 43.5 mL, 1.5 eq) and TBAF (1 M in THF, 46.3 mL, 0.2 eq) drop-wise at 20° C. under N₂. After the addition, the mixture was heated and stirred at 45° C. for 16 h. TLC showed the reaction was complete. The two reaction mixtures were combined and poured into H₂O (1 L), and extracted with EtOAc (1 L×3). The combined organic layers were washed with brine (500 mL×3), dried over Na₂SO₄, filtered, and concentrated under reduced pressure to give a residue. The residue was purified by flash silica gel chromatography (ISCO®; 300 g SepaFlash® Silica Flash Column, Eluent of 0˜50% Ethyl acetate/petroleum ether gradient @ 200 mL/min) to give N-(trans)-(2-cyanocyclopentyl)-4-methyl-benzenesulfonamide (100 g, 81.8% yield) as a yellow solid. ¹H NMR (CDCl₃, 400 MHz): δ=7.82 (d, J=8.3 Hz, 2H), 7.35 (d, J=8.1 Hz, 2H), 5.64 (d, J=5.9 Hz, 1H), 3.76 (m, 1H), 2.84 (dt, J=8.5, 6.0 Hz, 1H), 2.45 (s, 3H), 2.04-2.16 (m, 1H), 1.82-2.02 (m, 2H), 1.66-1.80 (m, 2H), 1.40-1.55 ppm (m, 1H).

### Preparation of tert-butyl ((trans)-2-cyanocyclopentyl)(tosyl)carbamate

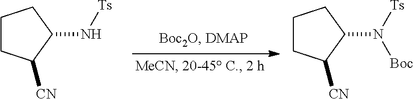 [SMILES: Cc1ccc(S(=O)(=O)N[C@H]2CCC[C@@H]2C#N)cc1 ; Cc1ccc(S(=O)(=O)N(C(=O)OC(C)(C)C)[C@H]2CCC[C@@H]2C#N)cc1]

To a solution of N-(trans)-(2-cyanocyclopentyl)-4-methyl-benzenesulfonamide (95 g, 359 mmol, 1 eq) in MeCN (1520 mL) was added Boc₂O (117 g, 539 mmol, 124 mL, 1.5 eq) and DMAP (13.1 g, 108 mmol, 0.3 eq) at 20° C. under N₂. After the addition, the mixture was heated and stirred at 45° C. for 2 h. TLC showed the reaction was complete. The reaction mixture was concentrated under reduced pressure to give a residue. The residue was purified by flash silica gel chromatography (ISCO®; 220 g SepaFlash® Silica Flash Column, Eluent of 0˜30% Ethyl acetate/petroleum ether gradient @ 200 mL/min) to give tert-butyl ((trans)-2-cyanocyclopentyl)(tosyl)carbamate (130 g, 99.2% yield) as an off-white solid. ¹H NMR (CDCl₃, 400 MHz): δ=7.87 (d, J=8.3 Hz, 2H), 7.33 (d, J=8.1 Hz, 2H), 5.13 (m, 1H), 3.49-3.58 (m, 1H), 2.45 (s, 3H), 2.23-2.33 (m, 1H), 2.11-2.20 (m, 2H), 1.89-2.00 (m, 2H), 1.76-1.87 (m, 1H), 1.36 ppm (s, 9H).

### Preparation of tert-butyl ((trans)-2-cyanocyclopentyl)carbamate

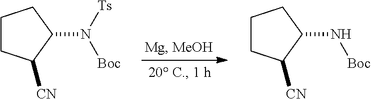 [SMILES: Cc1ccc(S(=O)(=O)N(C(=O)OC(C)(C)C)[C@H]2CCC[C@@H]2C#N)cc1 ; CC(C)(C)OC(=O)N[C@H]1CCC[C@@H]1C#N]

Three reactions were carried out in parallel. To each solution of tert-butyl ((trans)-2-cyanocyclopentyl)-N-(p-tolylsulfonyl)carbamate (32 g, 87.8 mmol, 1 eq) in MeOH (480 mL) was carefully added Mg (14.9 g, 612 mmol, 7 eq) at 20° C. under N₂ over 0.5 h. After the addition, the reaction was continue stirred at 20° C. for 0.5 h. TLC showed the reaction was complete. The three reaction mixtures were combined and diluted with DCM (500 mL) and poured into 1N aq. HCl (500 mL). The mixture was adjusted pH to 5 with 2N aq. HCl at 0° C. and extracted with DCM (300 mL×3). Then the organic phase was washed with saturated aq. NaHCO₃ (100 mL×3), the combined organic layers were washed with brine (100 mL×2), dried over Na₂SO₄, filtered, and concentrated under reduced pressure to give a residue. The residue was purified by flash silica gel chromatography (ISCO®; 220 g SepaFlash® Silica Flash Column, Eluent of 0˜30% Ethyl acetate/petroleum ether gradient @ 100 mL/min) to give tert-butyl ((trans)-2-cyanocyclopentyl)carbamate (49 g, 88.4% yield) as a white solid. ¹H NMR (CDCl₃, 400 MHz)): δ=4.12 (m, 1H), 2.82 (s, 1H), 2.06-2.20 (m, 2H), 1.88-2.00 (m, 1H), 1.81 (m, 2H), 1.38-1.50 ppm (m, 10H).

### Preparation of (trans)-2-aminocyclopentanecarbonitrile hydrochloride

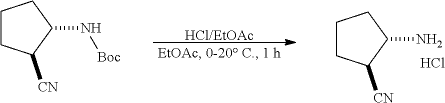 [SMILES: CC(C)(C)OC(=O)N[C@H]1CCC[C@@H]1C#N ; N#C[C@H]1CCC[C@@H]1N ; Cl]

To a mixture of tert-butyl ((trans)-2-cyanocyclopentyl)carbamate (62 g, 294 mmol, 1 eq) in EtOAc (900 mL) was added HCl/EtOAc (4 M, 221 mL, 3 eq) at 0° C. The mixture was stirred at 20° C. for 1 h. White solid was formed after reaction for 30 min. TLC showed the reaction was complete. The reaction mixture was filtered and the filter cake was concentrated in vacuum to give (trans)-2-aminocyclopentanecarbonitrile hydrochloride (36 g, 66% yield) as a white solid. ¹H NMR (D2O-d₆, 400 MHz) δ=3.90 (m, 1H), 3.06 (m, 1H), 2.16-2.30 (m, 2H), 1.90-1.99 (m, 1H), 1.80 (m, 2H), 1.62-1.73 ppm (m, 1H).

### Preparation of (trans)-2-[(2-chloro-5-methyl pyrimidin-4-yl)amino]cyclopentanecarbonitrile

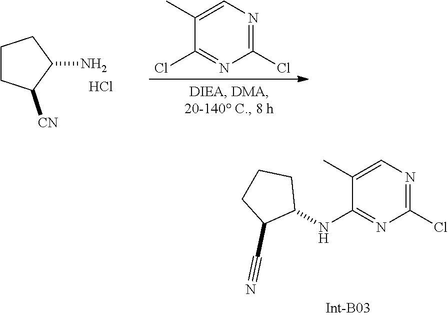

To a mixture of 2,4-dichloro-5-methyl-pyrimidine (22.4 g, 136 mmol, 1 eq) and (trans)-2-aminocyclopentanecarbonitrile hydrochloride (20 g, 136.4 mmol, 1 eq) in DMA (200 mL) was added DIEA (105 mmol, 818 mL, 6 eq) at 20° C. The mixture was heated and stirred at 140° C. for 8 h. TLC showed the reaction was complete. The reaction mixture was poured into H₂O (1 L), then the precipitate was formed. The precipitate was filtered to give (trans)-2-[(2-chloro-5-methyl-pyrimidin-4-yl)amino]cyclopentanecarbonitrile (26 g, 80% yield) as a white solid. ¹H NMR (CDCl₃, 400 MHz) δ=7.84 (s, 1H), 5.13 (m, 1H), 4.64-4.72 (m, 1H), 2.87-3.02 (m, 1H), 2.14-2.36 (m, 2H), 1.96-2.06 (m, 4H), 1.88-1.93 (m, 2H), 1.61-1.72 (m, 1H).

### Preparation of (trans)-2-((2-chloro-5-methylpyrimidin-4-yl)amino)cyclohexane-1-carbonitrile

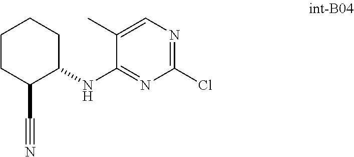

### Preparation of 7-tosyl-7-azabicyclo[4.1.0]heptane

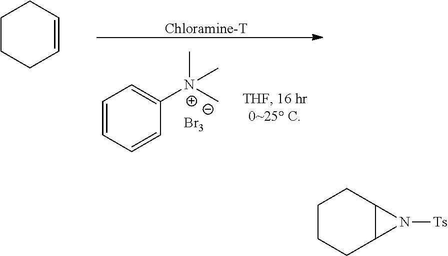

Two reactions were carried out in parallel. For each a mixture of cyclohexene (25 g, 304 mmol, 30.8 mL, 1 eq) in MeCN (500 mL) was added [chloro(p-tolylsulfonyl) amino]sodium (86.6 g, 380 mmol, 1.25 eq) and Phenyltrimethylammonium tribromide (11.4 g, 30.4 mmol, 0.1 eq) at 0° C., and then the mixture was heated and stirred at 25° C. for 16 h. TLC showed the reaction was complete. The two parallel reactions were combined and the reaction mixture was quenched by addition water (200 mL) at 0° C., and then extracted with DCM (200 mL×3). The combined organic layers were washed with brine (200 mL×2), dried over Na₂SO₄, filtered, and concentrated under reduced pressure to give a residue. The residue was purified by column chromatography (SiO₂, Petroleum ether/ethyl acetate=I/O to 20/1) to give 7-tosyl-7-azabicyclo[4.1.0]heptane (66 g, 43.1% yield) as colorless oil. ¹H NMR (CDCl₃-d₆, 400 MHz) δ 7.82 (d, J=8.3 Hz, 2H), 7.33 (d, J=8.2 Hz, 2H), 2.98 (d, J=1.1 Hz, 2H), 2.45 (s, 3H), 1.79 (m, 4H), 1.48-1.35 (m, 2H), 1.25-1.17 (m, 2H).

### Preparation of N-((trans)-2-cyanocyclohexyl)-4-methylbenzenesulfonamide

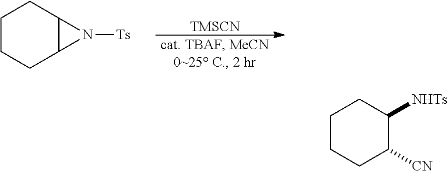

To a mixture of 7-(p-tolylsulfonyl)-7-azabicyclo[4, 1, 0] heptane (60 g, 238 mmol, 1 eq) in THF (600 mL) was added TMSCN (35.5 g, 358 mmol, 1.5 eq) and TBAF (1 M in THF, 47.7 mL, 0.2 eq) at 25° C., and then the mixture was stirred at 25° C. for 2 hr. TLC showed the reaction was complete. The reaction mixture was quenched by addition water (300 mL) at 0° C., and then extracted with DCM (300 mL×3). The combined organic layers were washed with brine (10 mL×2), dried over Na₂SO₄, filtered, and concentrated under reduced pressure to give N-((trans)-2-cyanocyclohexyl)-4-methylbenzenesulfonamide (70 g, crude) as a brown solid. ¹H NMR (CDCl₃-d₆, 400 MHz) δ 7.82 (d, J=8.4 Hz, 2H), 7.33 (d, J=8.0 Hz, 2H), 5.43-5.27 (m, 1H), 5.34 (br d, J=8.0 Hz, 1H), 3.39-3.35 (m, 1H), 2.67-2.65 (1H), 2.44 (s, 3H), 2.05-2.00 (m, 1H), 1.98-1.87 (m, 1H), 1.69-1.62 (m, 2H), 1.37-1.29 (m, 2H).

### Preparation of tert-butyl ((trans)-2-cyanocyclohexyl)-N-(p-tolylsulfonyl) carbamate

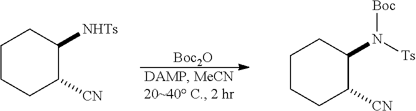

Two reactions were carried out in parallel. For each a mixture of N-((trans)-2-cyanocyclohexyl)-4-methyl-benzenesulfonamide (30 g, 107 mmol, 1 eq) in MeCN (300 mL) was added Boc₂O (35.2 g, 161 mmol, 1.5 eq) and DMAP (3.95 g, 32.3 mmol, 0.3 eq) at 20° C., and then the mixture was stirred at 40° C. for 2 hr. TLC showed the reaction was complete. The two parallel reactions were combined and quenched by addition water (300 mL) at 0° C., and then extracted with DCM (300 mL×3). The combined organic layers were washed with brine (300 mL×2), dried over Na₂SO₄, filtered, and concentrated under reduced pressure to give a residue. The residue was purified by column chromatography (SiO₂, Petroleum ether/ethyl acetate=I/O to 1/1) to give tert-butyl ((trans)-2-cyanocyclohexyl)-N-(p-tolylsulfonyl) carbamate (72 g, 88% yield) as white solid. ¹H NMR (CDCl₃-d₆, 400 MHz) δ 7.93 (d, J=8.4 Hz, 2H), 7.32 (d, J=8.0 Hz, 2H), 4.55-4.48 (m, 1H), 3.66-3.59 (m, 1H), 2.43 (s, 3H), 2.27-2.23 (m, 2H), 2.05-1.97 (m, 1H), 1.80-1.68 (m, 3H), 1.53-1.48 (m, 1H), 1.34 (s, 9H), 1.33-1.22 (m, 1H).

### Preparation of tert-butyl ((trans)-2-cyanocyclohexyl) carbamate

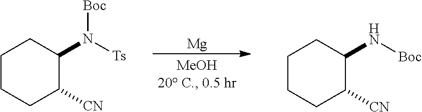

Two reactions were carried out in parallel. For each a mixture of tert-butyl ((trans)-2-cyanocyclohexyl)-N-(p-tolylsulfonyl) carbamate (26 g, 68.7 mmol, 1 eq) in MeOH (300 mL) was carefully added Mg (16.7 g, 687 mmol, 10 eq) at 20° C., and then the mixture was stirred at 20° C. for 0.5 h under N₂. TLC showed the reaction was complete. The two parallel reactions were combined the mixture was diluted with DCM (500 mL) and poured into aq. HCl (1N, 500 mL). Then, adjusted pH to 5 with aq. HCl (2N) at 0° C. and extracted with DCM (300 mL×3). Then the organic phase was washed with saturated aq. NaHCO₃. The combined organic layers were washed with brine (200 mL×2), dried over Na₂SO₄, filtered, and concentrated under reduced pressure to give a residue. The residue was purified by column chromatography (SiO₂, Petroleum ether/ethyl acetate=1/0 to 2/1) to give tert-butyl (trans)-2-cyanocyclohexyl) carbamate (28 g, 90% yield) as white solid. ¹H NMR (CDCl₃-d₆, 400 MHz) δ 4.64 (s, 1H), 3.69 (d, J=6.0 Hz, 1H), 2.60 (s, 1H), 2.15-1.96 (m, 2H), 1.81-1.61 (m, 3H), 1.47 (s, 9H), 1.42-1.22 (m, 3H).

### Preparation of (trans)-2-aminocyclohexanecarbonitrile hydrochloride

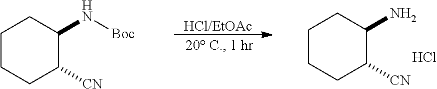 [SMILES: CC(C)(C)OC(=O)N[C@@H]1CCCC[C@H]1C#N ; N#C[C@@H]1CCCC[C@H]1N ; Cl]

To a mixture of tert-butyl (trans)-2-cyanocyclohexyl) carbamate (38 g, 169 mmol, 1 eq) in EtOAc (100 mL) was added HCl/EtOAc (4 M, 300 mL) at 20° C., and then the mixture was stirred at 20° C. for 1 h. TLC showed the reaction was complete. The reaction mixture was concentrated under reduced pressure to give (trans)-2-aminocyclohexanecarbonitrile hydrochloride (27 g, 99.2% yield) as a white solid. ¹H NMR (DMSO-d₆, 400 MHz) δ 8.62 (s, 2H), 3.34 (s, 1H), 3.12-3.01 (m, 1H), 2.52 (d, J=2.0 Hz, 1H), 2.13-2.00 (m, 2H), 1.74-1.55 (m, 3H), 1.50-1.22 (m, 3H).

### Preparation of (trans)-2-((2-chloro-5-methylpyrimidin-4-yl)amino)cyclohexane-1-carbonitrile

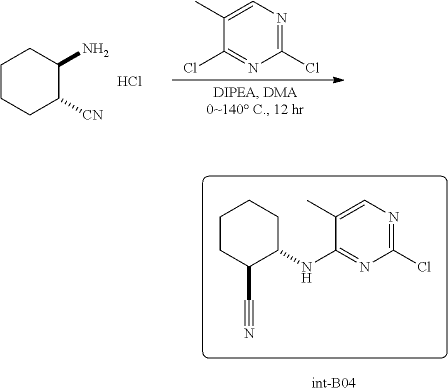

Two reactions were carried out in parallel. For each a mixture of (trans)-2-aminocyclohexanecarbonitrile hydrochloride (13 g, 80.9 mmol, 1 eq) in DMA (150 mL) was added 2,4-dichloro-5-methyl-pyrimidine (13.2 g, 80.9 mmol, 1 eq) and DIPEA (62.7 g, 485 mmol, 6 eq) at 20° C., and then the mixture was stirred at 140° C. for 12 h. TLC showed the reaction was complete. The two parallel reactions were combined and quenched by addition water (100 mL) at 0° C., and then extracted with DCM (100 mL×3). The combined organic layers were washed with brine (100 mL×2), dried over Na₂SO₄, filtered, and concentrated under reduced pressure to give a residue. The residue was purified by column chromatography (SiO₂, Petroleum ether/Ethyl acetate=1/0 to 0/1) to give (trans)-2-((2-chloro-5-methylpyrimidin-4-yl)amino)cyclohexane-1-carbonitrile (30 g, 73.9% yield) as a colorless oil. ¹H NMR (CDCl₃-d₆, 400 MHz) δ 7.87 (s, 1H), 4.86 (d, J=8.0 Hz, 1H), 4.44-4.30 (m, 1H), 2.85-2.75 (m, 1H), 2.17-2.15 (m, 2H), 2.02 (s, 3H), 1.88-1.73 (m, 3H), 1.61-1.49 (m, 1H), 1.46-1.30 (m, 2H).

### Preparation of (trans)-3-((2-chloro-5-methylpyrimidin-4-yl)amino)tetrahydro-2H-pyran-4-carbonitrile

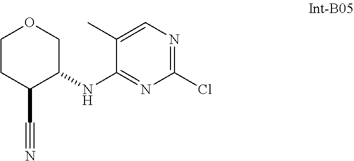

### Preparation of 7-tosyl-3-oxa-7-azabicyclo[4,1,0]heptane

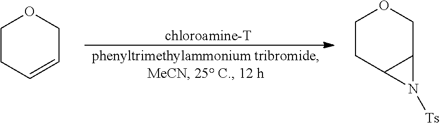 [SMILES: C1=CCOCC1 ; Cc1ccc(S(=O)(=O)N2C3CCOCC32)cc1]

Two reactions were carried out in parallel. A mixture of 3,6-dihydro-2H-pyran (100 g, 1.18 mol, 1 eq) in MeCN (2 L) was added phenyltrimethylammonium tribromide, (67 g, 179 mmol, 0.15 eq) and chloroamine-T (406 g, 1.78 mol, 1.5 eq) in portions under N₂. Then the mixture was stirred at 25° C. for 12 hr under N₂ atmosphere. TLC showed the reaction was complete. The combined reaction mixtures were quenched by addition H₂O (1 L) at 25° C. and extracted with MTBE (2 L×3) and the combined organic layers were washed with brine (1 L×2), dried over Na₂SO₄, filtered, and concentrated under reduced pressure to give a residue. The residue was triturated with MTBE (1 L) at 25° C. for 2 h, then filtered and the filtrate was concentrated under reduced pressure to give 7-tosyl-3-oxa-7-azabicyclo[4,1,0]heptane (750 g, crude) as yellow oil.

### Preparation of N-(trans)-(4-cyanotetrahydro-2H-pyran-3-yl)-4-methylbenzenesulfonamide

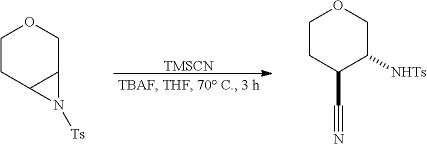

Six reactions were carried out in parallel. To each solution of 7-tosyl-3-oxa-7-azabicyclo[4,1,0]heptane (100 g crude, 395 mmol, 1 eq) in THF (1 L) was added TBAF (1 M in THF, 178 mL, 0.45 eq) and TMSCN (137 g, 1.4 mol, 173 mL, 3.5 eq) in portions under N₂. Then the mixture was stirred at 70° C. for 3 hr under N₂ atmosphere. TLC showed the reaction was complete. The six reaction mixtures were combined and quenched by addition H₂O (3 L) at 25° C., and extracted with EtOAc (3 L×3). The combined organic layers were washed with brine (3 L×2), dried over Na₂SO₄, filtered, and concentrated under reduced pressure to give a residue. The residue was purified by column chromatography (SiO₂, Petroleum ether/ethyl acetate=1/1 to 0/1) to give crude product, which was triturated with MTBE (1 L) at 25° C. for 2 h and filtered to give N-(trans)-(4-cyanotetrahydro-2H-pyran-3-yl)-4-methylbenzenesulfonamide (320 g) as a yellow solid. ¹H NMR (CDCl₃, 400 MHz) δ=7.78 (d, J=8.4 Hz, 2H), 7.35 (d, J=8.4 Hz, 2H), 5.33 (d, J=8.0 Hz, 1H), 3.85 (dd, J=2.4, 12.0 Hz, 1H), 3.77-3.74 (m, 2H), 3.47-3.42 (m, 1H), 3.36-3.32 (m, 1H), 3.06-3.03 (m, 1H), 2.45 (s, 3H), 2.23-2.16 (m, 1H), 1.82-1.76 (m, 1H).

### Preparation of tert-butyl-((trans)-(4-cyanotetrahydro-2H-pyran-3-yl)(tosyl)carbamate

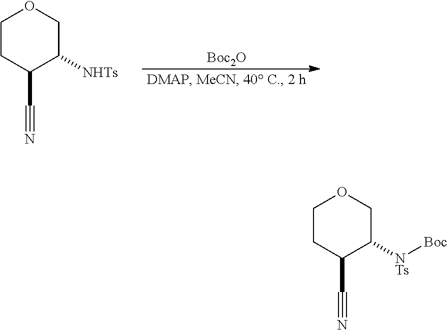

Four reactions were carried out in parallel. To each solution of N-(trans)-(4-cyanotetrahydro-2H-pyran-3-yl)-4-methylbenzenesulfonamide (80 g, 285 mmol, 1 eq) in MeCN (600 mL) was added Boc₂O (93 g, 428 mmol, 98 mL, 1.5 eq) and DMAP (10.5 g, 86 mmol, 0.3 eq) in portions under N₂. The mixture was stirred at 40° C. for 2 hr. TLC showed the reaction was complete. The four reaction mixtures were combined and quenched by addition H₂O (2 L) at 25° C. and concentrated under reduced pressure to remove MeCN, then extracted with EtOAc (2 L×3) and the combined organic layers were washed with brine (2 L×2), dried over Na₂SO₄, filtered, and concentrated under reduced pressure to give a residue. The residue was purified by column chromatography (SiO₂, Petroleum ether/ethyl acetate=1/0 to 3/1) to give tert-butyl-((trans)-(4-cyanotetrahydro-2H-pyran-3-yl)(tosyl)carbamate (300 g, 69.1% yield) as a yellow solid. ¹H NMR (CDCl₃, 400 MHz) δ=7.91 (d, J=8.0 Hz, 2H), 7.33 (d, J=8.0 Hz, 2H), 4.74-4.68 (m, 1H), 4.05-3.89 (m, 4H), 3.41-3.34 (m, 1H), 2.45 (s, 3H), 2.17-2.08 (m, 2H), 1.36 (s, 9H).

### Preparation of tert-butyl-((trans)-4-cyanotetrahydro-2H-pyran-3-yl)carbamate

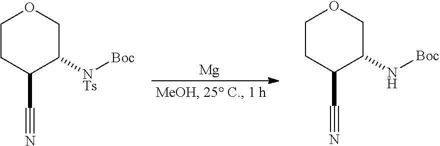

Six reactions were carried out in parallel. To each solution of tert-butyl ((trans)(4-cyanotetrahydro-2H-pyran-3-yl)(tosyl)carbamate (50 g, 131 mmol, 1 eq) in MeOH (800 mL) was added Mg (26 g, 1.1 mol, 8.2 eq) in portions carefully at 25° C. Accompanied by generation of heat (exothermic) and bubbling, the resulting suspension was stirred at 25° C. for 1 h. TLC showed the reaction was complete. The six reactions were combined and poured into ice-water (2.5 L), then adjusted pH to 5 with aq. HCl (2N) and extracted with EtOAc (1.5 L×3). The combined organic layers were washed with brine (3 L×2), dried over Na₂SO₄, filtered, and concentrated under reduced pressure to give tert-butyl ((trans)-4-cyanotetrahydro-2H-pyran-3-yl)carbamate (170 g, 95.5% yield) as a white solid. ¹H NMR (DMSO-d₆, 400 MHz) δ=7.18 (d, J=9.2 Hz, 1H), 3.78 (m, 1H), 3.71 (dd, J=4.4, 11.2 Hz, 1H), 3.57-3.54 (m, 1H), 3.23-3.17 (m, 1H), 2.96 (t, J=10.8 Hz, 1H), 2.88-2.81 (m, 1H), 2.06-2.01 (m, 1H), 1.83-1.72 (m, 1H), 1.39 (s, 9H).

### Preparation of (trans)-3-aminotetrahydro-2H-pyran-4-carbonitrile (hydrogen chloride)

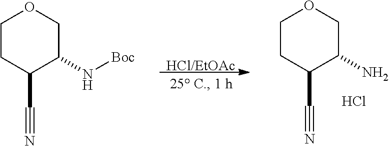 [SMILES: CC(C)(C)OC(=O)N[C@H]1COCC[C@@H]1C#N ; N#C[C@H]1CCOC[C@@H]1N ; Cl]

HCl/EtOAc (4 M, 1.7 L) was slowly added to compound tert-butyl ((trans)-4-cyanotetrahydro-2H-pyran-3-yl)carbamate (170 g, 751 mmol, 1 eq) at 25° C. After addition, the reaction was stirred at 25° C. for 1 h. Solid precipitated out after 30 min reaction. TLC showed the reaction was complete. The reaction mixture was filtered and the filter cake was concentrated under reduced pressure to give (trans)-3-aminotetrahydro-2H-pyran-4-carbonitrile (91 g, 74.5% yield, HCl salt) as a white solid. ¹H NMR (DMSO-d₆, 400 MHz) δ=8.74 (s, 3H), 3.89 (dd, J=2.8, 12.0 Hz, 1H), 3.75-3.65 (m, 1H), 3.62-3.42 (m, 4H), 2.25-2.15 (m, 1H), 1.85-1.75 (m, 1H).

### Preparation of (trans)-3-((2-chloro-5-methylpyrimidin-4-yl)amino)tetrahydro-2H pyran-4-carbonitrile

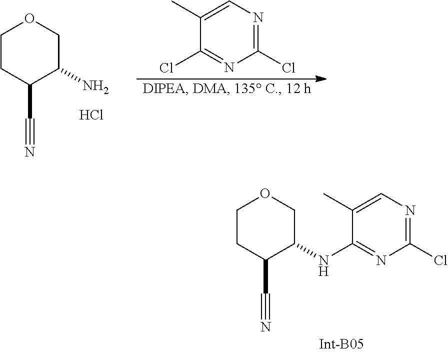

To a solution of (trans)-3-aminotetrahydro-2H-pyran-4-carbonitrile (82 g, 504 mmol, 1 eq, HCl) in DMA (1 L) was added DIPEA (391 g, 3.02 mol, 525 mL, 6 eq) and 2,4-dichloro-5-methyl-pyrimidine (82.5 g, 504 mmol, 1 eq), and then the mixture was stirred at 135° C. for 12 hr. TLC showed the reaction was complete. The reaction mixture was quenched by addition H₂O (4 L) at 20° C., then extracted with EtOAc (2 L×3). The combined organic layers were washed with brine (2 L×2), dried over Na₂SO₄, filtered, and concentrated under reduced pressure to give a residue. The residue was triturated with EtOAc (500 mL) at 20° C. for 12 hr to give (trans)-3-((2-chloro-5-methylpyrimidin-4-yl)amino)tetrahydro-2H-pyran-4-carbonitrile (75 g, 58.9% yield) as a white solid. ¹H NMR (DMSO-d₆, 400 MHz) δ=7.92 (s, 1H), 7.27 (d, J=8.4 Hz, 1H), 4.38-4.29 (m, 1H), 3.89-3.81 (m, 2H), 3.28-3.27 (m, 1H), 3.23-3.16 (m, 1H), 3.10 (t, J=10.8 Hz, 1H), 2.15-2.11 (m, 1H), 2.00 (s, 3H), 1.96-1.86 (m, 1H). This material (20 g) was separated by SFC (column: DAICEL CHIRALPAK IC (250 mm×50 mm, 10 um); mobile phase: [0.1% NH₃H₂O IPA]; B %: 60%-60%, 8.5 min) to give (trans)-3-((2-chloro-5-methylpyrimidin-4-yl)amino)tetrahydro-2H-pyran-4-carbonitrile (8.95 g, 94.5% purity, 97.56% ee, first peak, Rt=2.160 min) stereoisomer one (int-B05-P1) as a white solid. ¹H NMR (DMSO-d₆, 400 MHz) δ=7.91 (s, 1H), 7.27 (d, J=8.8 Hz, 1H), 4.35-4.33 (m, 1H), 3.89-3.81 (m, 2H), 3.33-3.08 (m, 3H), 2.15-2.11 (m, 1H), 2.00 (s, 3H), 1.96-1.86 (m, 1H) and (trans)-3-((2-chloro-5-methylpyrimidin-4-yl)amino)tetrahydro-2H-pyran-4-carbonitrile (9.05 g, 99.19% purity, 98.9% ee, second peak, Rt=3.141 min) stereoisomer two (int-B05-P2) as a white solid. ¹H NMR (DMSO-d₆, 400 MHz) δ=7.92 (s, 1H), 7.27 (d, J=8.8 Hz, 1H), 4.35-4.33 (m, 1H), 3.89-3.81 (m, 2H), 3.33-3.08 (m, 3H), 2.15-2.11 (m, 1H), 2.00 (s, 3H), 1.96-1.86 (m, 1H).

### Preparation of (trans)-3-((2,5-dichloropyrimidin-4-yl)amino)tetrahydro-2H-pyran-4-carbonitrile

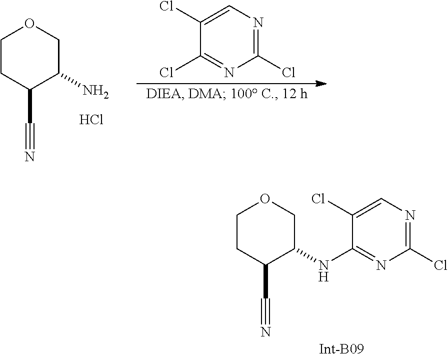 [SMILES: N#C[C@H]1CCOC[C@@H]1N ; Cl ; Clc1ncc(Cl)c(Cl)n1 ; N#C[C@H]1CCOC[C@@H]1Nc1nc(Cl)ncc1Cl]

A mixture of (trans)-3-aminotetrahydro-2H-pyran-4-carbonitrile (2 g, 15.8 mmol, 1 eq, HCl salt), 2,4,5-trichloropyrimidine (2.91 g, 15.8 mmol, 1 eq), DIEA (4.10 g, 31.7 mmol, 5.52 mL, 2 eq) in DMA (20 mL) was degassed and purged with N₂ for 3 times, and then the mixture was stirred at 100° C. for 12 h under N₂ atmosphere. TLC showed the reaction was complete. The reaction mixture was diluted with H₂O (200 mL) and extracted with ethyl acetate (100 mL×3). The combined organic layers were washed with brine (80 mL×2), dried over Na₂SO₄, filtered, and concentrated under reduced pressure to give a residue. The residue was purified by flash silica gel chromatography (ISCO®; 40 g SepaFlash® Silica Flash Column, Eluent of 0˜65% Ethyl acetate/petroleum ether gradient @ 80 mL/min) to obtain (trans)-3-((2,5-dichloropyrimidin-4-yl)amino)tetrahydro-2H-pyran-4-carbonitrile (1.2 g, 27.7% yield) as light yellow oil. ¹H NMR (400 MHz, DMSO-d₆) δ=8.29 (s, 1H), 7.98 (d, J=8.8 Hz, 1H), 4.40-4.28 (m, 1H), 3.90-3.83 (m, 2H), 3.29-3.11 (m, 3H), 2.18-2.08 (m, 1H), 1.93-1.85 (m, 1H).

### Preparation of (trans)-3-((2-chloro-5 fluoropyrimidin-4-yl)amino)tetrahydro-2H-pyran-4-carbonitrile

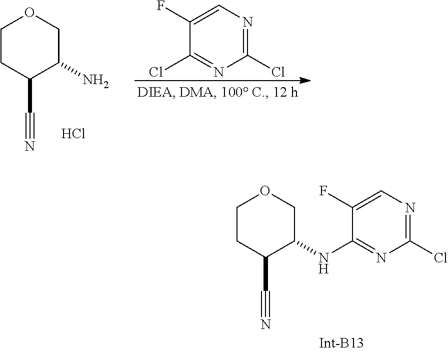 [SMILES: N#C[C@H]1CCOC[C@@H]1N ; Cl ; Fc1cnc(Cl)nc1Cl ; N#C[C@H]1CCOC[C@@H]1Nc1nc(Cl)ncc1F]

To a solution of (trans)-3-aminotetrahydro-2H-pyran-4-carbonitrile (500 mg, 3.07 mmol, 1 eq, HCl salt) in DMA (5 mL) was added DIEA (1.6 g, 12.3 mmol, 2 mL, 4 eq) and 2,4-dichloro-5-fluoro-pyrimidine (513 mg, 3.07 mmol, 1 eq) at 25° C. and then the mixture was stirred at 100° C. for 12 h. TLC showed the reaction was complete. The reaction mixture was quenched by addition H₂O (20 mL) at 25° C., then extracted with EtOAc (10 mL×3) and the combined organic layers were washed with brine (10 mL×2), dried over Na₂SO₄, filtered, and concentrated under reduced pressure to give a residue. The residue was purified by flash silica gel chromatography (ISCO®; 12 g SepaFlash® Silica Flash Column, Eluent of 0˜25% Ethyl acetate/petroleum ether gradient @ 50 mL/min) to give (trans)-3-((2-chloro-5-fluoropyrimidin-4-yl)amino)tetrahydro-2H-pyran-4-carbonitrile (310 mg, 39% yield) as a yellow solid. ¹H NMR (DMSO-d₆, 400 MHz) δ=8.37 (d, J=8.4 Hz, 1H), 8.20 (d, J=3.2 Hz, 1H), 4.27-4.24 (m, 1H), 3.88-3.84 (m, 2H), 3.31-3.07 (m, 3H), 2.14-2.10 (m, 1H), 1.98-1.87 (m, 1H).

### Preparation of (trans)-2-[(2-chloro-5 fluoro-pyrimidin-4-yl) amino]cyclohexanecarbonitrile

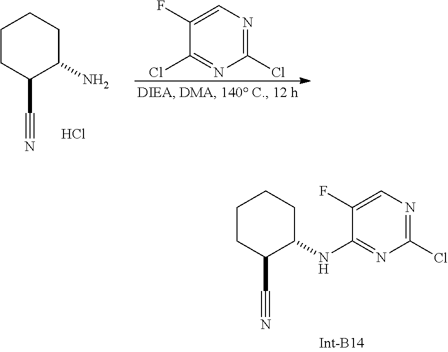 [SMILES: N#C[C@H]1CCCC[C@@H]1N ; Cl ; Fc1cnc(Cl)nc1Cl ; N#C[C@H]1CCCC[C@@H]1Nc1nc(Cl)ncc1F]

A mixture of (trans)-2-aminocyclohexanecarbonitrile (1 g, 6.22 mmol, 1 eq, HCl salt) in DMA (10 mL) was added DIEA (4.83 g, 37.3 mmol, 6.5 mL, 6 eq) and 2,4-dichloro-5-fluoro-pyrimidine (1.04 g, 6.22 mmol, 1 eq) at 25° C., and then the mixture was stirred at 140° C. for 12 h. TLC showed the reaction was complete. The reaction mixture was quenched by addition H₂O (40 mL) at 25° C., and then extracted with EtOAc (20 mL×3). The combined organic layers were washed with brine (50 mL×2), dried with anhydrous Na₂SO₄, filtered, and concentrated in vacuum to give a crude product. The crude product was triturated with MTBE (10 mL) at 25° C. for 30 min to afford (trans)-2-[(2-chloro-5-fluoro-pyrimidin-4-yl) amino]cyclohexanecarbonitrile (1.2 g, 75.8% yield) as a brown solid. ¹H NMR (DMSO-d₆, 400 MHz) δ 8.34 (d, J=8.8 Hz, 1H), 8.15 (d, J=3.2 Hz, 1H), 4.21-4.14 (m, 1H), 2.86-2.79 (m, 1H), 2.13-2.09 (m, 1H), 1.89-1.85 (m, 1H), 1.72-1.64 (m, 3H), 1.36-1.25 (m, 2H), 1.21-1.10 (m, 1H).

### Preparation of (trans)-2-((2,5-dichloropyrimidin-4-yl)amino)cyclohexanecarbonitrile

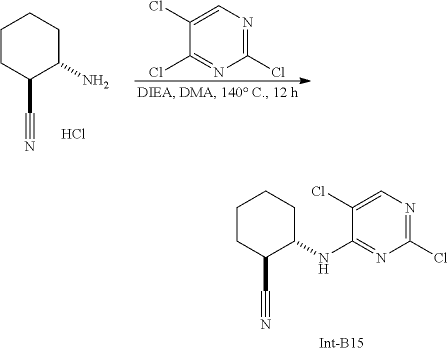 [SMILES: N#C[C@H]1CCCC[C@@H]1N ; Cl ; Clc1ncc(Cl)c(Cl)n1 ; N#C[C@H]1CCCC[C@@H]1Nc1nc(Cl)ncc1Cl]

A mixture of (trans)-2-aminocyclohexanecarbonitrile (2.00 g, 12.4 mmol, 1 eq, HCl salt), 2,4,5-trichloropyrimidine (2.28 g, 12.4 mmol, 1 eq), DIEA (9.65 g, 74.7 mmol, 13.0 mL, 6 eq) in DMA (20 mL) was degassed and purged with N₂ for 3 times, and then the mixture was stirred at 140° C. for 12 h under N₂ atmosphere. TLC showed the reaction was complete. The reaction mixture was diluted with H₂O (50 mL) and extracted with ethyl acetate (30 mL×3). The combined organic layers were washed with brine (30 mL×2), dried over Na₂SO₄, filtered, and concentrated under reduced pressure to give a residue. The residue was purified by flash silica gel chromatography (ISCO®; 40 g SepaFlash® Silica Flash Column, Eluent of 0˜35% Ethyl acetate/petroleum ether gradient @80 mL/min) to obtain (trans)-2-((2,5-dichloropyrimidin-4-yl)amino)cyclohexanecarbonitrile (2 g, 59.2% yield) as a yellow solid. ¹H NMR (400 MHz, DMSO-d₆) δ=8.24 (s, 1H), 7.98 (d, J=9.2 Hz, 1H), 4.30-4.17 (m, 1H), 2.99 (m, 1H), 2.16-2.05 (m, 1H), 1.88-1.78 (m, 1H), 1.73-1.58 (m, 3H), 1.44-1.30 (m, 2H), 1.21-1.06 (m, 1H).

### Preparation of 3-((2-chloro-5-(trifluoromethyl)pyrimidin-4-yl)amino)tetrahydro-2H-pyran-4-carbonitrile

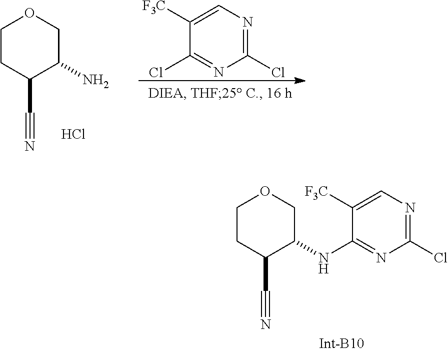 [SMILES: N#C[C@H]1CCOC[C@@H]1N ; Cl ; FC(F)(F)c1cnc(Cl)nc1Cl ; N#C[C@H]1CCOC[C@@H]1Nc1nc(Cl)ncc1C(F)(F)F]

To a solution of 2-aminotetrahydropyran-4-carbonitrile (2 g, 12.3 mmol, 1 eq, HCl) in THE (20 mL) was added DIEA (4.77 g, 36.9 mmol, 6.4 mL, 3 eq) and 2,4-dichloro-5-(trifluoromethyl)pyrimidine (3.20 g, 14.7 mmol, 1.2 eq) at 25° C. The mixture was stirred at 25° C. for 16 hr. LCMS showed the reaction was complete and 2 peaks with desired MS observed. The reaction mixture was concentrated under reduced pressure to give a residue. The residue was purified by prep-HPLC (column: Xtimate C18 10 u 250 mm*80 mm; mobile phase: [water (NH₄HCO₃)-ACN]; B %: 20%-55%, 30 min) to give (trans)-2-[[2-chloro-5-(trifluoromethyl)pyrimidin-4-yl]amino]tetrahydropyran-4-carbonitrile (800 mg, 21.2% yield) as a yellow solid ¹H NMR (400 MHz, DMSO-d₆) δ=8.56-8.44 (m, 1H), 7.85 (d, J=8.4 Hz, 1H), 4.60-4.43 (m, 1H), 3.91-3.78 (m, 2H), 3.45-3.40 (m, 1H), 3.28-3.17 (m, 2H), 2.18-2.07 (m, 1H), 1.96-1.86 (m, 1H).

### Preparation of 2-((2-chloro-5-(trifluoromethyl)pyrimidin-4-yl)amino)cyclohexane-1-carbonitrile

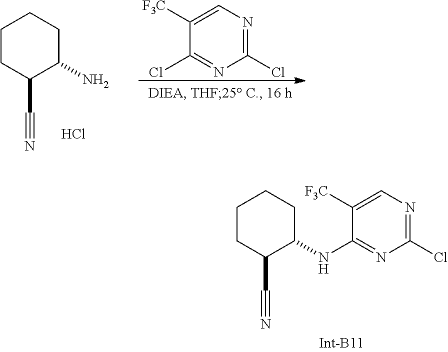 [SMILES: N#C[C@H]1CCCC[C@@H]1N ; Cl ; FC(F)(F)c1cnc(Cl)nc1Cl ; N#C[C@H]1CCCC[C@@H]1Nc1nc(Cl)ncc1C(F)(F)F]

To a solution of (trans)-2-aminocyclohexanecarbonitrile (2 g, 12.4 mmol, 1 eq, HCl) in THF (20 mL) was added DIEA (4.83 g, 37.3 mmol, 3 eq) and 2,4-dichloro-5-(trifluoromethyl)pyrimidine (3.24 g, 14.9 mmol, 1.2 eq) at 25° C. The mixture was stirred at 25° C. for 16 hr. LCMS showed the starting material was consumed completely and desired MS was detected. The reaction mixture was concentrated under reduced pressure to give a residue. The residue was purified by prep-HPLC (column: Xtimate C18 10 u 250 mm*80 mm; mobile phase: [water (NH₄HCO₃)-ACN]; B %: 35%-75%, 35 min) to give (trans)-2-((2-chloro-5-(trifluoromethyl)pyrimidin-4-yl)amino)cyclohexane-1-carbonitrile (900 mg, 23.7% yield) as a yellow solid ¹H NMR (400 MHz, DMSO-d₆) δ=8.47 (s, 1H), 7.86 (d, J=8.8 Hz, 1H), 4.47-4.34 (m, 1H), 3.08 (m, 1H), 2.17-2.06 (m, 1H), 1.85-1.75 (m, 1H), 1.75-1.57 (m, 3H), 1.51-1.28 (m, 2H), 1.21-1.06 (m, 1H).

### Preparation of (trans)-2-[[2-chloro-5-(trifluoromethyl)pyrimidin-4-yl]amino]cyclopentanecarbonitrile

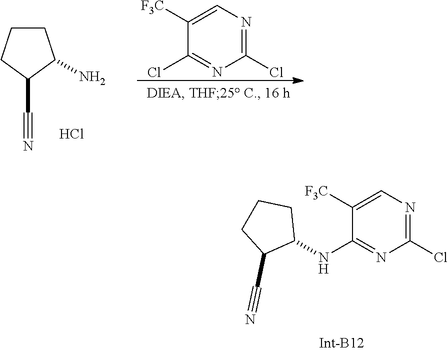 [SMILES: N#C[C@H]1CCC[C@@H]1N ; Cl ; FC(F)(F)c1cnc(Cl)nc1Cl ; N#C[C@H]1CCC[C@@H]1Nc1nc(Cl)ncc1C(F)(F)F]

To a solution of (trans)-2-aminocyclopentanecarbonitrile (2 g, 13.6 mmol, 1 eq, HCl) in THF (20 mL) was added 2,4-dichloro-5-(trifluoromethyl)pyrimidine (2.96 g, 13.6 mmol, 1 eq) and DIEA (5.29 g, 40.9 mmol, 7.13 mL, 3 eq) at 25° C. The mixture was stirred at 25° C. for 16 h. TLC showed the reaction was complete. The reaction mixture was concentrated under reduced pressure to give a residue. The residue was purified by prep-HPLC (column: Agela DuraShell C18 250*70 mm*10 um; mobile phase: [water (NH₄HCO₃)-ACN]; B %: 30%-60%, 20 min) to afford (trans)-2-[[2-chloro-5-(trifluoromethyl)pyrimidin-4-yl]amino] cyclopentanecarbonitrile (1.37 g, 34.5% yield) as a white solid. ¹H NMR (CDCl₃, 400 MHz) δ 8.34 (s, 1H), 5.35 (s, 1H), 4.85-4.75 (m, 1H), 2.92-2.83 (m, 1H), 2.42-2.31 (m, 1H), 2.27-2.17 (m, 1H), 2.14-2.03 (m, 1H), 2.01-1.88 (m, 2H), 1.71-1.61 (m, 1H).

### Synthetic Examples (for Compounds in Table 1 and Table 2)

### 1. Preparation of (trans)-2-((2-((1-hydroxy-1,3-dihydrobenzo[c][1,2]oxaborol-5-yl)amino)-5-methylpyrimidin-4-yl)amino)cyclopentane-1-carbonitrile

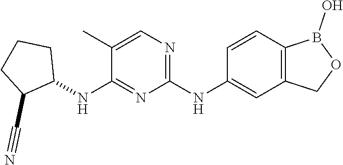

### 1.1 Preparation of methyl 2-bromo-5-((4-(((trans)-2-cyanocyclopentyl)amino)-5-methylpyrimidin-2-yl)amino)benzoate

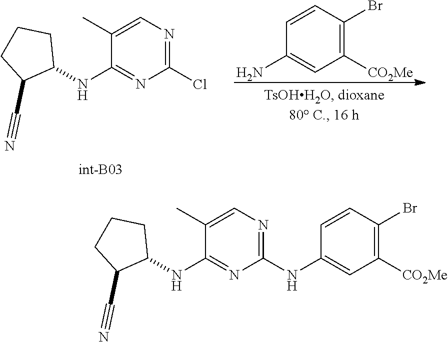 [SMILES: Cc1cnc(Cl)nc1N[C@H]1CCC[C@@H]1C#N ; COC(=O)c1cc(N)ccc1Br ; COC(=O)c1cc(Nc2ncc(C)c(N[C@H]3CCC[C@@H]3C#N)n2)ccc1Br]

A mixture of (trans)-2-[(2-chloro-5-methyl-pyrimidin-4-yl)amino]cyclopentanecarbonitrile (2.00 g, 8.45 mmol, 1 eq), methyl 5-amino-2-bromo-benzoate (2.14 g, 9.29 mmol, 1.1 eq) and TsOH·H₂O (3.21 g, 16.9 mmol, 2 eq) in dioxane (60 mL) was heated and stirred at 80° C. for 12 h. TLC showed the reaction was complete. The reaction mixture was cooled to room temperature and concentrated in vacuum to give a residue. The residue was quenched with saturated aq. Na₂CO₃ (50 mL) and extracted with EtOAc (20 mL×3). The combined organics were dried over Na₂SO₄, filtered, and concentrated in vacuum to give a residue. The residue was triturated with a mixture solvent of MTBE (50 mL) and EtOAc (10 mL) to give methyl 2-bromo-5-((4-(((trans)-2-cyanocyclopentyl)amino)-5-methylpyrimidin-2-yl)amino)benzoate (1.50 g, 41% yield) as a gray solid. ¹H NMR (CDCl₃, 400 MHz) δ 8.05 (s, 1H), 7.80 (s, 1H), 7.76-7.73 (m, 1H), 7.57 (d, J=8.4 Hz, 1H), 7.12 (s, 1H), 4.68-4.62 (m, 1H), 4.50-4.48 (m, 1H), 3.94 (s, 3H), 2.88-2.84 (m, 1H), 2.36-2.34 (m, 1H), 2.15-2.08 (m, 2H), 1.97 (s, 3H), 1.93-1.91 (m, 1H), 1.64-1.62 (m, 1H).

### 1.2 Preparation of methyl 5-((4-(((trans)-2-cyanocyclopentyl)amino)-5-methylpyrimidin-2-yl)amino)-2-(5,5-dimethyl-1,3,2-dioxaborinan-2-yl)benzoate

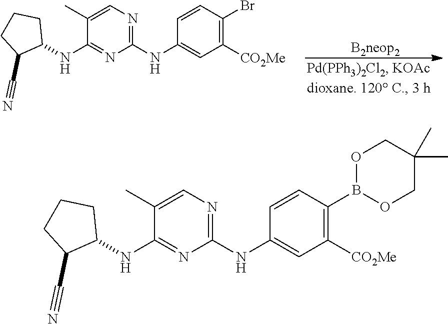 [SMILES: COC(=O)c1cc(Nc2ncc(C)c(N[C@H]3CCC[C@@H]3C#N)n2)ccc1Br ; COC(=O)c1cc(Nc2ncc(C)c(N[C@H]3CCC[C@@H]3C#N)n2)ccc1B1OCC(C)(C)CO1]

A mixture of methyl 2-bromo-5-((4-(((trans)-2-cyanocyclopentyl)amino)-5-methylpyrimidin-2-yl)amino)benzoate (500 mg, 1.16 mmol, 1 eq), 2-(5,5-dimethyl-1,3,2-dioxaborinan-2-yl)-5,5-dimethyl-1,3,2-dioxaborinane (656 mg, 2.90 mmol, 2.5 eq), Pd(PPh₃)₂Cl₂ (81 mg, 116 umol, 0.1 eq) and KOAc (285 mg, 2.90 mmol, 2.5 eq) in dioxane (20 mL) was heated and stirred at 120° C. for 3 h. TLC showed the reaction was complete. The reaction mixture was cooled to room temperature and filtered. Then the filtrate was concentrated in vacuum to give a residue. The residue was triturated with a mixture solvent of MTBE (50 mL), EtOAc (5 mL) and Petroleum ether (10 mL) to give methyl 5-((4-(((trans)-2-cyanocyclopentyl)amino)-5-methylpyrimidin-2-yl)amino)-2-(5,5-dimethyl-1,3,2-dioxaborinan-2-yl)benzoate (300 mg, crude) as a gray solid. ¹H NMR (CDCl₃, 400 MHz) δ 8.14 (s, 1H), 7.82-7.80 (m, 2H), 7.51-7.49 (m, 1H), 7.15 (s, 1H), 4.68-4.65 (m, 1H), 4.46-4.45 (m, 1H), 3.92 (s, 3H), 3.79 (s, 4H), 3.61-3.60 (m, 6H), 2.92-2.87 (m, 1H), 2.36-2.35 (m, 1H), 2.15-2.13 (m, 1H), 2.13-2.05 (m, 1H), 1.96 (s, 3H), 1.93-1.92 (m, 2H), 1.63-1.62 (m, 1H), 1.10 (s, 6H).

### 1.3 Preparation of (trans)-2-((2-((1-hydroxy-1,3-dihydrobenzo[c][1,2]oxaborol-5-yl)amino)-5-methylpyrimidin-4-yl)amino)cyclopentane-1-carbonitrile

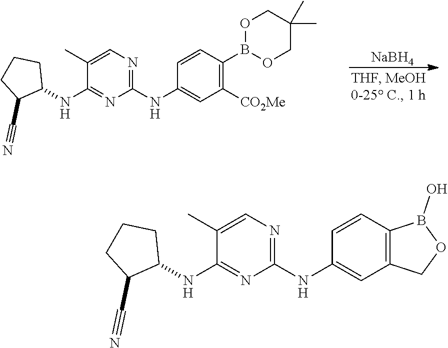 [SMILES: COC(=O)c1cc(Nc2ncc(C)c(N[C@H]3CCC[C@@H]3C#N)n2)ccc1B1OCC(C)(C)CO1 ; "NaBH4" ; Cc1cnc(Nc2ccc3c(c2)COB3O)nc1N[C@H]1CCC[C@@H]1C#N]

To a solution of methyl 5-[[4-[((trans)-2-cyanocyclopentyl)amino]-5-methyl-pyrimidin-2-yl]amino]-2-(5,5-dimethyl-1,3,2-dioxaborinan-2-yl)benzoate (500 mg, 1.08 mmol, 1 eq) in THF (10 mL) and MeOH (2 mL) was added NaBH₄ (204 mg, 5.40 mmol, 5 eq) portion-wise at 0° C. After the addition, the reaction was allowed to warm to 25° C. and stirred at 25° C. for 1 h. LCMS showed the reaction was complete and desired MS observed. The reaction mixture was quenched with aq. HCl (1 N, 5 mL) carefully and diluted with H₂O (10 mL). The mixture was extracted with EtOAc (10 mL×3). The combined organics were washed with brine, dried over Na₂SO₄, filtered, and concentrated in vacuum to give a residue. The residue was triturated with a mixture solvent of MeCN (5 mL) and H₂O (2 mL) to give (trans)-2-((2-((1-hydroxy-1,3-dihydrobenzo[c][1,2]oxaborol-5-yl)amino)-5-methylpyrimidin-4-yl)amino)cyclopentane-1-carbonitrile (48.3 mg, 11.6% yield) as a white solid. ¹HNMR (DMSO-d₆, 400 MHz) δ 12.27 (s, 1H), 10.63 (s, 1H), 9.14 (s, 1H), 8.43 (s, 1H), 7.84 (s, 1H), 7.75-7.73 (m, 2H), 7.48-7.46 (m, 1H), 5.00 (s, 2H), 4.76-4.74 (m, 1H), 3.26-3.24 (m, 1H), 2.18-2.17 (m, 1H), 2.12-2.09 (m, 1H), 2.04 (s, 3H), 1.86-1.71 (m, 4H). MS (ESI): mass calcd. For C18H21BClN5O2 349.17, m/z found 350.1 [M+H]⁺. HPLC: 94.26% (220 nm), 92.97% (254 nm).

### 2. Preparation of (trans)-2-((2-((1-hydroxy-7-methyl-1,3-dihydrobenzo[c][1,2]oxaborol-5-yl)amino)-5-methylpyrimidin-4-yl)amino)cyclopentane-1-carbonitrile

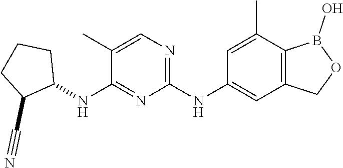

### 2.1 Preparation of methyl 2-bromo-5-[[4-[(trans)-(2-cyanocyclopentyl)amino]-5-methyl-pyrimidin-2-yl]amino]-3-methyl-benzoate

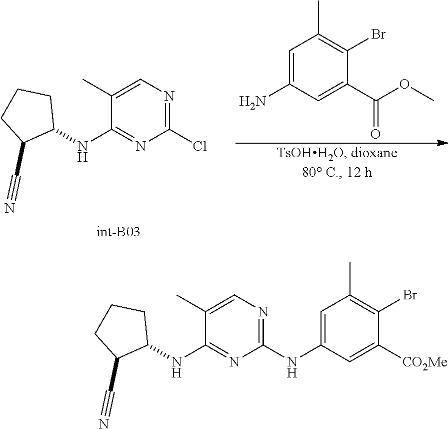 [SMILES: Cc1cnc(Cl)nc1N[C@H]1CCC[C@@H]1C#N ; COC(=O)c1cc(N)cc(C)c1Br ; COC(=O)c1cc(Nc2ncc(C)c(N[C@H]3CCC[C@@H]3C#N)n2)cc(C)c1Br]

To a mixture of (trans)-2-[(2-chloro-5-methyl-pyrimidin-4-yl)amino]cyclopentanecarbonitrile (1.40 g, 5.91 mmol, 1 eq) and methyl 5-amino-2-bromo-3-methyl-benzoate (1.59 g, 6.51 mmol, 1.1 eq) in dioxane (15 mL) was added TsOH·H₂O (2.25 g, 11.8 mmol, 2 eq) in one portion at 20° C. under N₂. The mixture was heated and stirred at 80° C. for 12 h. TLC showed the reaction was complete. The reaction mixture was filtered and the white filter cake was washed with EtOAc (10 mL) to give methyl 2-bromo-5-[[4-[(trans)-(2-cyanocyclopentyl)amino]-5-methyl-pyrimidin-2-yl]amino]-3-methyl-benzoate (1.2 g, 45.6% yield) as a white solid. ¹H NMR (CDCl₃, 400 MHz) δ 10.41 (s, 1H), 8.36 (s, 1H), 7.83 (s, 1H), 7.82 (s, 1H), 7.64 (s, 1H), 4.76-4.68 (m, 1H), 3.86 (s, 3H), 3.21-3.19 (m, 1H), 2.42 (s, 3H), 2.20-2.18 (m, 1H), 2.06-2.03 (m, 4H), 1.85-1.69 (m, 4H).

### 2.2 Preparation of methyl 5-[[4-[(2-cyanocyclopentyl)amino]-5-methyl-pyrimidin-2-yl]amino]-2-(5,5-dimethyl-1,3,2-dioxaborinan-2-yl)-3-methyl-benzoate

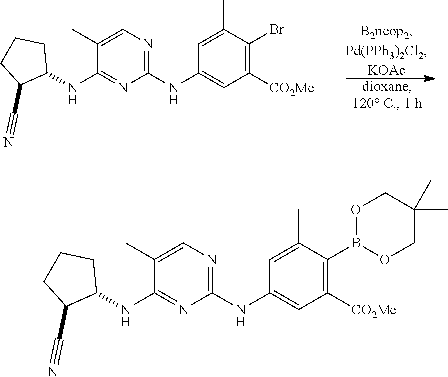 [SMILES: COC(=O)c1cc(Nc2ncc(C)c(N[C@H]3CCC[C@@H]3C#N)n2)cc(C)c1Br ; COC(=O)c1cc(Nc2ncc(C)c(N[C@H]3CCC[C@@H]3C#N)n2)cc(C)c1B1OCC(C)(C)CO1]

To a mixture of methyl 2-bromo-5-[[4-[((trans)-2-cyanocyclopentyl)amino]-5-methyl-pyrimidin-2-yl]amino]-3-methyl-benzoate (600 mg, 1.35 mmol, 1 eq) and 2-(5,5-dimethyl-1,3,2-dioxaborinan-2-yl)-5,5-dimethyl-1,3,2-dioxaborinane (762 mg, 3.38 mmol, 2.5 eq) in dioxane (6 mL) was added KOAc (331 mg, 3.38 mmol, 2.5 eq) and Pd(PPh₃)₂Cl₂ (94 mg, 135 umol, 0.1 eq) in one portion at 20° C. under N₂. The mixture was stirred at 120° C. for 1 h. TLC showed the reaction was complete. The reaction mixture was filtered and the filtrate was concentrated under reduced pressure to give a residue. The crude product was triturated with MTBE (20 mL) at 20° C. for 10 min to give methyl 5-[[4-[((trans)-2-cyanocyclopentyl)amino]-5-methyl-pyrimidin-2-yl]amino]-2-(5,5-dimethyl-1,3,2-dioxaborinan-2-yl)-3-methyl-benzoate (900 mg, crude) as an off-white solid.

### 2.3 Preparation of (trans)-2-((2-((1-hydroxy-7-methyl-1,3-dihydrobenzo[c][1,2]oxaborol-5-yl)amino)-5-methylpyrimidin-4-yl)amino)cyclopentane-1-carbonitrile

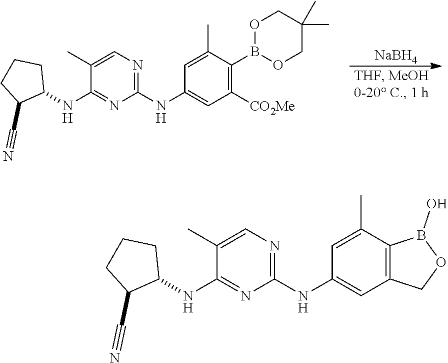 [SMILES: COC(=O)c1cc(Nc2ncc(C)c(N[C@H]3CCC[C@@H]3C#N)n2)cc(C)c1B1OCC(C)(C)CO1 ; "NaBH4" ; Cc1cnc(Nc2cc(C)c3c(c2)COB3O)nc1N[C@H]1CCC[C@@H]1C#N]

To a mixture of methyl 5-[[4-[((trans)-2-cyanocyclopentyl)amino]-5-methyl-pyrimidin-2-yl]amino]-2-(5,5-dimethyl-1,3,2-dioxaborinan-2-yl)-3-methyl-benzoate (900 mg, 1.89 mmol, 1 eq) in THE (10 mL) and MeOH (1 mL) was added NaBH₄ (178 mg, 4.71 mmol, 2.5 eq) in portions at 0° C. After the addition, the reaction was allowed to warm to 20° C. and stirred at 20° C. for 1 h. TLC showed the reaction was complete. The reaction mixture was adjusted pH=6 by aq. HCl (2N). The mixture was extracted with EtOAc (10 mL×3). The combined organic layers were washed with brine (10 mL×2), dried over anhydrous Na₂SO₄, filtered, and concentrated under reduced pressure to give a residue. The residue was purified by prep-HPLC (column: Phenomenex luna C18 80*40 mm*3 um; mobile phase: [water (0.04% HCl)-ACN]; B %: 15%-35%, 7 min) to give (trans) 2-[[2-[(1-hydroxy-7-methyl-3H-2,1-benzoxaborol-5-yl)amino]-5-methyl-pyrimidin-4-yl]amino]cyclopentanecarbonitrile (30 mg) as a white solid. ¹H NMR (DMSO-d₆, 400 MHz) δ 12.62 (s, 1H), 10.47 (s, 1H), 8.88 (s, 1H), 8.42 (d, J=8.0 Hz, 1H), 7.82 (s, 1H), 7.46 (s, 1H), 7.28 (s, 1H), 4.96 (s, 2H), 4.80-4.72 (m, 1H), 3.27-3.22 (m, 1H), 2.44 (s, 3H), 2.23-2.19 (m, 1H), 2.12-2.10 (m, 1H), 2.03 (s, 3H), 1.88-1.70 (m, 4H). MS (ESI): mass calcd. For 19H23BClN5O2, 363.11, m/z found 364.2 [M+H]⁺. HPLC: 96.69% (220 nm), 96.77% (254 nm).

### 3. Preparation of (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile

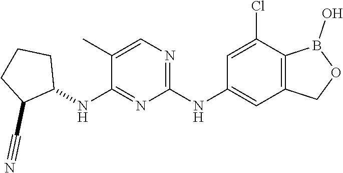

### 3.1 Preparation of methyl 2-amino-3-chloro-5-nitro-benzoate

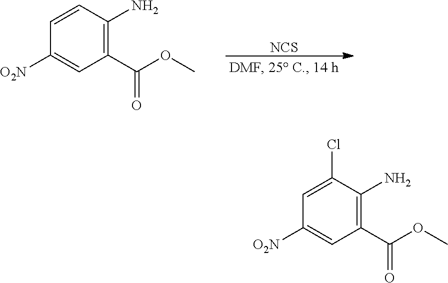

Three reactions were carried out in parallel. For each a solution of methyl 2-amino-5-nitro-benzoate (10 g, 51.0 mmol, 1 eq) in DMF (100 mL) was added NCS (8.85 g, 66.2 mmol, 1.3 eq) at 25° C. The mixture was stirred at 25° C. for 14 h. TLC showed the reaction was complete. The contents of three parallel reactions were combined and the mixture was poured into water (300 mL) at 0° C., then the yellow precipitate formed and was collected. The yellow precipitate was washed with H₂O (80 mL×2) and petroleum ether (50 mL×2) to give methyl 2-amino-3-chloro-5-nitro-benzoate (34.1 g, 96.7% yield) as a yellow solid. ¹H NMR (DMSO-d₆, 400 MHz) δ 8.58 (d, J=2.4 Hz, 1H), 8.36 (d, J=2.4 Hz, 1H), 3.89 (s, 3H).

### 3.2 Preparation of methyl 2-bromo-3-chloro-5-nitro-benzoate

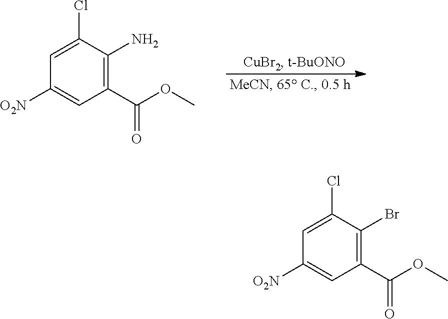

Three reactions were carried out in parallel. For each a mixture of t-BuONO (8.94 g, 86.7 mmol, 10.3 mL, 2 eq) and CuBr₂ (14.5 g, 65.1 mmol, 3.1 mL, 1.5 eq) in MeCN (100 mL) was added methyl 2-amino-3-chloro-5-nitro-benzoate (10 g, 43.3 mmol, 1 eq) in portions at 65° C. The mixture was stirred at 65° C. for 30 min. TLC showed the reaction was complete. The three parallel reactions were combined and poured into H₂O (350 mL) and extracted with EtOAc (100 mL×3). The combined organic layers were washed with saturated aq. Na₂SO₃ (100 mL×3). Then the combined organic layers were washed with brine (100 mL×2), dried over anhydrous Na₂SO₄, filtered, and concentrated under reduced pressure to give methyl 2-bromo-3-chloro-5-nitro-benzoate (33 g, 86.1% yield) as a yellow solid. ¹H NMR (CDCl₃, 400 MHz) δ 8.44-8.42 (m, 2H), 4.02 (s, 3H).

### 3.3 Preparation of methyl 5-amino-2-bromo-3-chloro-benzoate

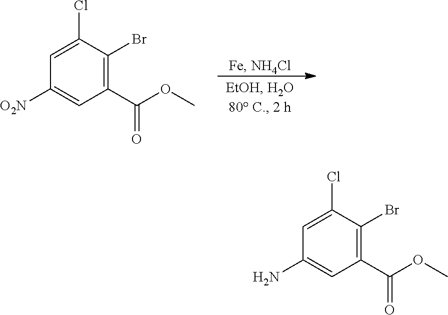

Three reactions were carried out in parallel. For each a mixture of methyl 2-bromo-3-chloro-5-nitro-benzoate (11 g, 37.3 mmol, 1 eq) in EtOH (120 mL) and H₂O (40 mL) was added Fe (6.26 g, 112 mmol, 3 eq) and NH₄Cl (3.00 g, 56.0 mmol, 1.5 eq) in one portion at 25° C. The mixture was heated to 80° C. and stirred at 80° C. for 2 h. TLC showed the reaction was complete. The three parallel reactions were combined and filtered followed by concentration under reduced pressure to give a residue. The residue was quenched with water (500 mL) and extracted with EtOAc (150 mL×3). The combined organic layers were washed with brine (250 mL×3), dried over Na₂SO₄, filtered, and concentrated under reduced pressure to give methyl 5-amino-2-bromo-3-chloro-benzoate (28 g, 94.5% yield) as yellow oil. ¹H NMR (CDCl₃, 400 MHz) δ 6.90 (d, J=2.8 Hz, 1H), 6.86 (d, J=2.8 Hz, 1H), 3.92 (s, 3H), 3.90-3.80 (m, 2H).

### 3.4 Preparation of (5-amino-2-bromo-3-chloro phenyl)methanol

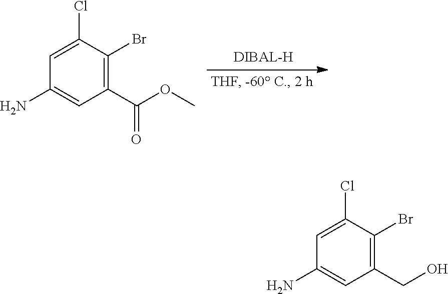

To a solution of methyl 5-amino-2-bromo-3-chloro-benzoate (10 g, 37.8 mmol, 1 eq) in THF (150 mL) was added DIBAL-H (1 M, 113 mL, 3 eq) drop-wise at −60° C. The mixture was stirred at −60° C. for 2 h. TLC showed the reaction was complete. The reaction mixture was quenched by addition aq. seignette salt (500 mL) at 0° C., and then extracted with EtOAc (70 mL×3). The combined organic layers were washed with brine (200 mL×2), dried over Na₂SO₄, filtered, and concentrated under reduced pressure to give a residue. The residue was triturated with MTBE (30 mL) at 25° C. for 5 min to give (5-amino-2-bromo-3-chloro-phenyl)methanol (8.2 g, 91.7% yield) as a yellow solid. ¹H NMR (CDCl₃, 400 MHz) δ 6.76 (d, J=2.8 Hz, 1H), 6.65 (d, J=2.8 Hz, 1H), 5.54 (s, 2H), 5.39 (t, J=5.6 Hz, 1H), 4.37 (d, J=5.6 Hz, 2H).

### 3.5 Preparation of (trans)-2-[[2-[4-bromo-3-chloro-5-(hydroxymethyl)anilino]-5-methyl-pyrimidin-4-yl]amino]cyclopentanecarbonitrile

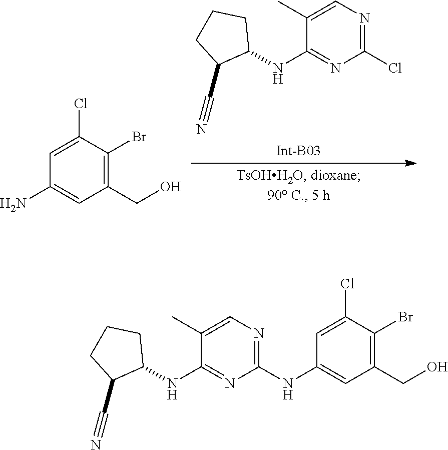

Five reactions were carried out in parallel. For each a solution of (5-amino-2-bromo-3-chloro-phenyl)methanol (0.5 g, 2.11 mmol, 1 eq) and (trans)-2-[(2-chloro-5-methyl-pyrimidin-4-yl)amino]cyclopentanecarbonitrile (525 mg, 2.22 mmol, 1.05 eq) in dioxane (15 mL) was added TsOH·H₂O (603 mg, 3.17 mmol, 1.5 eq) at 25° C. The mixture was heated to 90° C. and stirred at 90° C. for 5 h. TLC showed the reaction was complete. The five parallel reactions were combined and quenched by addition of saturated aq. NaHCO₃ (80 mL) at 0° C. and extracted with EtOAc (30 mL×3). The combined organic layers were washed with brine (80 mL×2), dried over Na₂SO₄, filtered, and concentrated under reduced pressure to give a residue. The residue was triturated with MTBE (15 mL) at 25° C. for 10 min to give (trans)-2-[[2-[4-bromo-3-chloro-5-(hydroxymethyl)anilino]-5-methyl-pyrimidin-4-yl]amino]cyclopentanecarbonitrile (2.8 g, 60.6% yield) as a white solid. ¹H NMR (DMSO-d₆, 400 MHz) δ 9.29 (s, 1H), 8.12 (s, 1H), 7.91 (d, J=2.8 Hz, 1H), 7.76 (s, 1H), 6.81 (d, J=8.8 Hz, 1H), 5.50 (t, J=5.6 Hz, 1H), 4.87-4.78 (m, 1H), 4.48 (d, J=5.6 Hz, 2H), 3.15-3.08 (m, 1H), 2.24-2.12 (m, 2H), 1.94 (s, 3H), 1.89-1.83 (m, 1H), 1.82-1.74 (m, 2H), 1.57-1.47 (m, 1H).

### 3.6 Preparation of (trans)-2-[[2-[4-bromo-3-[[tert-butyl(dimethyl)silyl]oxymethyl]-5-chloro-anilino]-5-methyl-pyrimidin-4-yl]amino]cyclopentanecarbonitrile

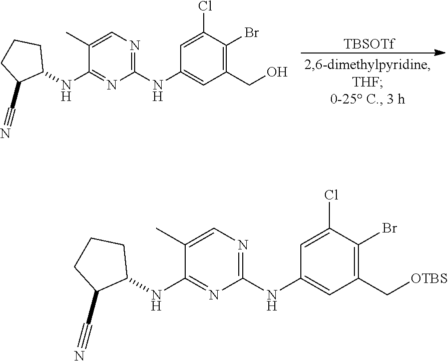

To a solution of (trans)-2-[[2-[4-bromo-3-chloro-5-(hydroxymethyl)anilino]-5-methyl-pyrimidin-4-yl]amino]cyclopentanecarbonitrile (2.8 g, 6.41 mmol, 1 eq) and 2,6-dimethylpyridine (2.08 g, 19.2 mmol, 2.3 mL, 3 eq) in THE (60 mL) was added TBSOTf (2.54 g, 9.62 mmol, 2.2 mL, 1.5 eq) at 0° C. The mixture was stirred at 25° C. for 3 h. TLC showed the reaction was complete. The reaction mixture was quenched by addition saturated aq. NH₄Cl (80 mL) at 0° C., and then extracted with EtOAc (30 mL×3). The combined organic layers were washed with brine (40 mL×2), dried over Na₂SO₄, filtered, and concentrated under reduced pressure to give a residue. The residue was triturated with EtOAc (30 mL) at 25° C. for 20 min to give (trans)-2-[[2-[4-bromo-3-[[tert-butyl(dimethyl)silyl]oxymethyl]-5-chloro-anilino]-5-methyl-pyrimidin-4-yl]amino]cyclopentanecarbonitrile (2.5 g, 70.7% yield) as a white solid. ¹H NMR (DMSO-d₆, 400 MHz) δ 9.39 (s, 1H), 8.30 (d, J=2.8 Hz, 1H), 7.75 (s, 1H), 7.72 (d, J=2.8 Hz, 1H), 6.82 (d, J=8.0 Hz, 1H), 4.78 (m, 1H), 4.66 (s, 2H), 3.13 (q, J=8.4 Hz, 1H), 2.25-2.09 (m, 2H), 1.95 (s, 3H), 1.87-1.73 (m, 3H), 1.60-1.51 (m, 1H), 0.92 (s, 9H), 0.10 (s, 6H).

### 3.7 Preparation of [2-[[tert-butyl(dimethyl)silyl]oxymethyl]-6-chloro-4-[[4-[(trans)-(2-cyanocyclopentyl)amino]-5-methyl pyrimidin-2-yl]amino]phenyl]boronic acid

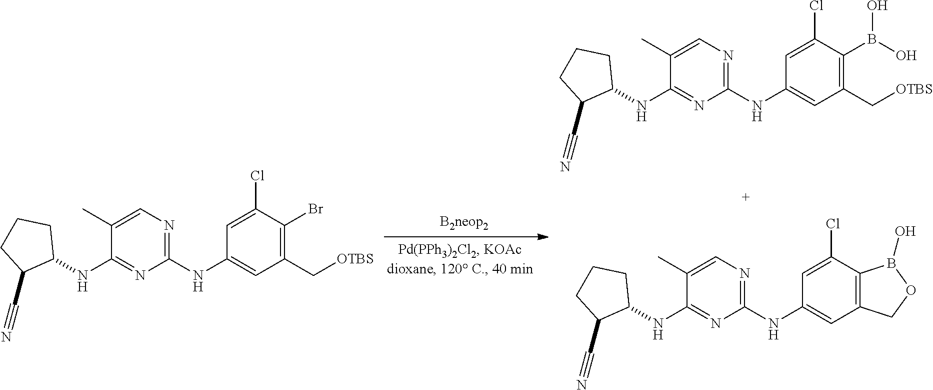

Six reactions were carried out in parallel. For each a mixture of (trans)-2-[[2-[4-bromo-3-[[tert-butyl(dimethyl)silyl]oxymethyl]-5-chloro-anilino]-5-methyl-pyrimidin-4-yl]amino]cyclopentanecarbonitrile (0.4 g, 726 umol, 1 eq), 2-(5,5-dimethyl-1,3,2-dioxaborinan-2-yl)-5,5-dimethyl-1,3,2-dioxaborinane (492 mg, 2.18 mmol, 3 eq), KOAc (214 mg, 2.18 mmol, 3 eq) and Pd(PPh₃)₂Cl₂ (51 mg, 72.6 umol, 0.1 eq) in dioxane (6 mL) was degassed and purged with N₂ for 3 times. Then the mixture was heated to 120° C. and stirred at 120° C. for 40 min under N₂ atmosphere. LCMS showed the reaction was complete and desired MS observed. The six parallel reactions were cooled to room temp, combined and filtered. The filtrate was concentrated under reduced pressure to give a residue. The residue was purified by prep-HPLC (column: Waters Xbridge BEH C18 250*50 mm*10 um; mobile phase: [water (NH₄HCO₃)-ACN]; B %: 50%-90%, 10 min) to give [2-[[tert-butyl(dimethyl)silyl]oxymethyl]-6-chloro-4-[[4-[(trans)-(2-cyanocyclopentyl)amino]-5-methyl-pyrimidin-2-yl]amino]phenyl]boronic acid and 2-((2-((7-chloro-1-hydroxy-1,3-dihydrobenzo[c][1,2]oxaborol-5-yl)amino)-5-methylpyrimidin-4-yl)amino)cyclopentane-1-carbonitrile (1.2 g, mixture) as a white solid. ¹H NMR (DMSO-d₆, 400 MHz) δ 9.11 (s, 1H), 8.04 (s, 2H), 7.91 (s, 1H), 7.73 (s, 1H), 7.51 (s, 1H), 6.78 (d, J=8.0 Hz, 1H), 4.80-4.70 (m, 1H), 4.63 (s, 2H), 3.20-3.10 (m, 1H), 2.25-2.09 (m, 2H), 1.95 (s, 3H), 1.87-1.73 (m, 3H), 1.60-1.51 (m, 1H), 0.94 (s, 9H), 0.13 (s, 6H).

### 3.8 Preparation of (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile

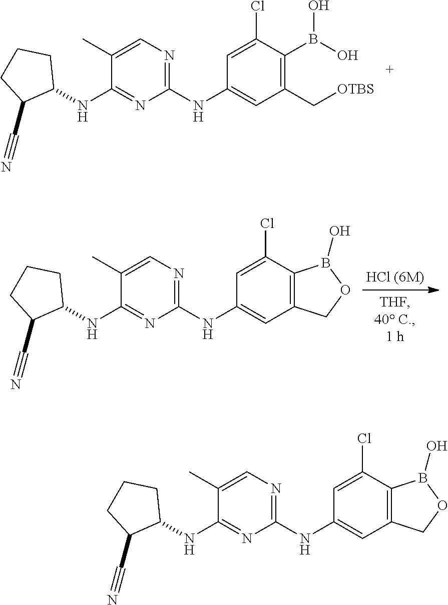

To a solution of [2-[[tert-butyl(dimethyl)silyl]oxymethyl]-6-chloro-4-[[4-[(2-cyanocyclopentyl)amino]-5-methyl-pyrimidin-2-yl]amino]phenyl]boronic acid and 2-((2-((7-chloro-1-hydroxy-1,3-dihydrobenzo[c][1,2]oxaborol-5-yl)amino)-5-methylpyrimidin-4-yl)amino)cyclopentane-1-carbonitrile (mixture, 1.2 g, 1.16 mmol, 1 eq) in THF (10 mL) was added HCl (6 M, 775 uL, 4 eq) at 0° C. The mixture was heated to 40° C. and stirred at 40° C. for 1 h. LCMS showed the reaction was complete and one main peak with desired mass was detected. The reaction was concentrated under reduced pressure to give a residue. The residue was triturated with MeCN (15 mL) at 25° C. for 10 min to give racemic product (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile (880 mg). The racemic product (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile (880 mg) was separated by SFC (column: DAICEL CHIRALPAK AD (250 mm*30 mm, 10 um); mobile phase: [0.1% NH₃H₂O IPA]; B %: 40%-40%, 6 min) to give (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile (stereoisomer one) (335.9 mg, 97.10% purity, 98.96% ee, first peak, Rt=3.898 min) as a white solid. ¹H NMR (DMSO-d₆, 400 MHz) δ 9.39 (s, 1H), 8.84 (s, 1H), 7.81 (s, 1H), 7.78 (s, 1H), 7.76 (s, 1H), 6.88 (d, J=8.0 Hz, 1H), 4.94 (s, 2H), 4.85-4.75 (m, 1H), 3.13 (q, J=8.4 Hz, 1H), 2.25-2.10 (m, 2H), 1.96 (s, 3H), 1.90-1.73 (m, 3H), 1.62-1.53 (m, 1H). MS (ESI): mass calcd. For C₁₈H₁₉BClN₅O₂ 383.13, m/z found 384.1 [M+H]⁺. HPLC: 97.10% (220 nm), 98.17% (254 nm); and 370 mg of stereoisomer two, containing isopropanol residue), which was further purified by pre-HPLC (column: Phenomenex Luna 80*30 mm*3 um; mobile phase: [water(HCl)-ACN]; B %: 5%-35%, 8 min) to remove isopropanol residue to obtain (trans)-2-((2-((7-chloro-1-hydroxy-1,3-dihydrobenzo[c][1,2]oxaborol-5-yl)amino)-5-methylpyrimidin-4-yl)amino)cyclopentane-1-carbonitrile (stereoisomer two) (332.9 mg, 97.31% purity, 97.80% ee, second peak, Rt=4.470 min) as a white solid. ¹H NMR (DMSO-d₆, 400 MHz) δ 10.40 (br s, 1H), 9.05 (br s, 1H), 8.10 (br s, 1H), 7.84 (s, 1H), 7.71 (s, 1H), 7.62 (s, 1H), 4.99 (s, 2H), 4.80-4.70 (m, 1H), 3.26-3.19 (m, 1H), 2.26-2.19 (m, 1H), 2.15-2.09 (m, 1H), 2.03 (s, 3H), 1.90-1.75 (m, 3H), 1.71-1.63 (m, 1H). MS (ESI): mass calcd. For C₁₈H₁₉BClN₅O₂ 383.13, m/z found 384.1 [M+H]⁺. HPLC: 97.31% (220 nm), 97.90% (254 nm).

### 4. Preparation of (trans)-2-((2-((7,8-dihydro-2H-1,6,9-trioxa-9a-borabenzo [cd]azulen-4-yl)amino)-5-methylpyrimidin-4-yl)amino)cyclopentane-1-carbonitrile

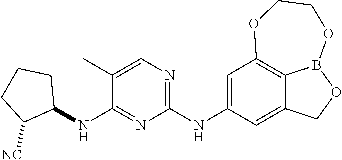

### 4.1 Preparation of methyl 2-amino-3-methoxy-benzoate

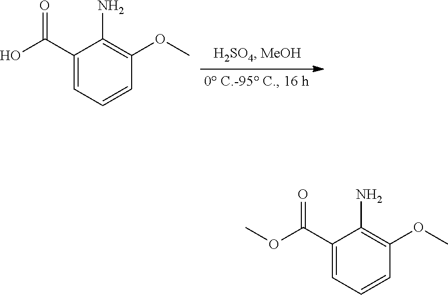

To a solution of 2-amino-3-methoxy-benzoic acid (40.0 g, 239 mmol, 1 eq) in MeOH (500 mL) was drop-wise added a solution of H₂SO₄ (1.32 mol, 70 mL, 5.5 eq) at 0° C. over 5 mins under N₂ at 0° C. After the addition, the reaction mixture was heated and stirred at 95° C. for 16 h. TLC showed the reaction was complete. The reaction mixture was adjusted pH to 7 with saturated aq. Na₂CO₃ (1 L) at 0° C. and extracted with ethyl acetate (500 mL×3). The combined organic phase was washed with brine (300 mL×2), dried with anhydrous Na₂SO₄, filtered, and concentrated in vacuum to give a residue. The residue was purified by column chromatography (SiO₂, Petroleum ether/ethyl acetate=10/1 to 5/1) to give methyl 2-amino-3-methoxy-benzoate (43 g, 99% yield) as a white solid. ¹H NMR (CDCl₃, 400 MHz) δ 7.40 (d, J=8.4 Hz, 1H), 7.77 (d, J=7.6 Hz, 1H), 6.50 (t, J=8.0 Hz, 1H), 5.94 (s, 2H), 3.79 (s, 6H).

### 4.2 Preparation of methyl 2-amino-5-iodo-3-methoxy-benzoate

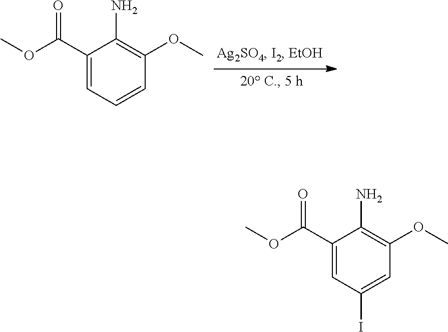

To a mixture of methyl 2-amino-3-methoxy-benzoate (43.0 g, 237 mmol, 1 eq) in EtOH (500 mL) was added Ag₂SO₄ (77.6 g, 249 mmol, 1.05 eq) and I₂ (63.2 g, 249 mmol, 1.05 eq) in one portion at 20° C. The mixture was stirred at 20° C. for 5 h. TLC showed the reaction was complete. The mixture was poured into ice-water (1 L) and stirred for 5 min. The aqueous phase was extracted with ethyl acetate (600 mL×3). The combined organic phase was washed with saturated aq. Na₂SO₃ (400 mL×2) and brine (150 mL×2), dried with anhydrous Na₂SO₄, filtered, and concentrated in vacuum to give a residue. The residue was purified by column chromatography (SiO₂, Petroleum ether/ethyl acetate=10/1 to 5/1) to give methyl 2-amino-5-iodo-3-methoxy-benzoate (45.0 g, 61% yield) as a white solid. ¹H NMR (DMSO-d₆, 400 MHz) δ 7.61 (s, 1H), 7.15 (s, 1H), 6.45 (s, 2H), 3.83 (s, 3H), 3.79 (s, 3H).

### 4.3 Preparation of methyl 2-bromo-5-iodo-3-methoxy-benzoate

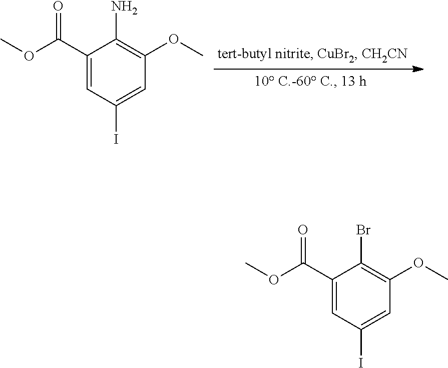

To a solution of methyl 2-amino-5-iodo-3-methoxy-benzoate (45.0 g, 146 mmol, 1 eq) in MeCN (600 mL) was added CuBr₂ (66.4 g, 297 mmol, 2.03 eq) at 25° C. The mixture was stirred at 25° C. for 20 min, and then, tert-butyl nitrite (264 mmol, 31 mL, 1.8 eq) was added dropwise over 10 min at 10° C. The reaction mixture was stirred at 10° C. for additional 30 min, and the mixture was heated and stirred at 60° C. for 12 h. The residue was poured into ice-water (1 L) and saturated aq. Na₂CO₃ (600 mL). The aqueous phase was extracted with ethyl acetate (500 mL×3). The combined organic phase was washed with saturated aq. Na₂SO₃ (500 mL) and brine (500 mL), dried with anhydrous Na₂SO₄, filtered, and concentrated in vacuum to give a residue. The residue was purified by column chromatography (SiO₂, Petroleum ether/ethyl acetate=10/1 to 5/1) to give methyl 2-bromo-5-iodo-3-methoxy-benzoate (33 g, 60% yield) as a white solid. ¹H NMR (DMSO-d₆, 400 MHz) δ 7.47 (s, 1H), 7.44 (s, 1H), 3.92 (s, 3H), 3.85 (s, 3H).

### 4.4 Preparation of methyl 2,5-dibromo-3-hydroxy-benzoate

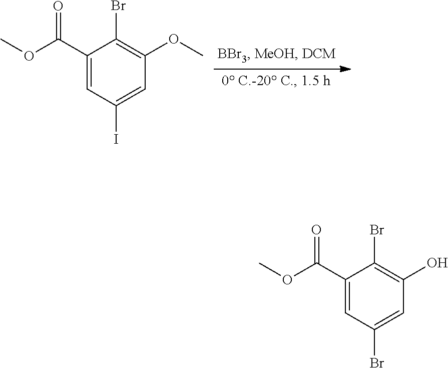

To a solution of methyl 2-bromo-5-iodo-3-methoxy-benzoate (33.0 g, 88.9 mmol, 1 eq) in DCM (400 mL) was slowly added BBr₃ (267 mmol, 25.7 mL, 3 eq) at 0° C. under N₂. After the addition, the reaction was allowed to warm to 20° C. and stirred for 1 h. TLC showed the reaction was complete. To the reaction mixture was slowly added MeOH (2.67 mol, 108 mL, 30 eq) and the resulting mixture was stirred at 20° C. for 30 min. The mixture was poured into ice-water (600 mL) and stirred for 5 min. The aqueous phase was extracted with DCM (350 mL×3). The combined organic phase was washed with brine (400 mL×2), dried with anhydrous Na₂SO₄, filtered, and concentrated in vacuum to give a residue. The residue was purified by column chromatography (SiO₂, Petroleum ether/ethyl acetate=10/1 to 5/1) to give methyl 2,5-dibromo-3-hydroxy-benzoate (17.0 g, 62% yield) as a white solid. ¹H NMR (DMSO-d₆, 400 MHz) δ 11.16 (s, 1H), 7.27 (d, J=2.4 Hz, 1H), 7.22 (d, J=2.4 Hz, 1H), 3.83 (s, 3H).

### 4.5 Preparation of methyl 2,5-dibromo-3-[2-[tert-butyl(dimethyl)silyl]oxyethoxy]benzoate

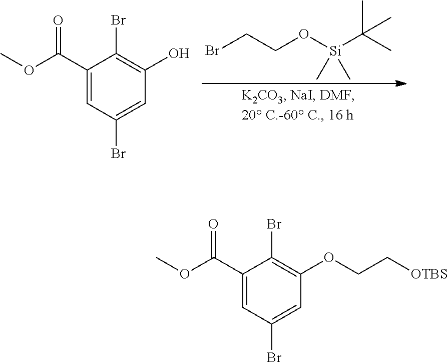

To a mixture of methyl 2,5-dibromo-3-hydroxy-benzoate (8.0 g, 25.8 mmol, 1 eq) and 2-bromoethoxy-tert-butyl-dimethyl-silane (9.26 g, 38.7 mmol, 1.5 eq) in DMF (80 mL) was added K₂CO₃ (7.13 g, 51.6 mmol, 2 eq) and NaI (774 mg, 5.16 mmol, 0.2 eq) in one portion at 20° C. under N₂. The mixture was heated to 60° C. and stirred for 16 h. TLC showed the reaction was complete. The mixture was poured into ice-water (150 mL) and stirred for 10 min. Then aqueous phase was extracted with ethyl acetate (80 mL×3). The combined organic phase was washed with brine (50 mL×2), dried with anhydrous Na₂SO₄, filtered, and concentrated in vacuum to give a residue. The residue was purified by column chromatography (SiO₂, Petroleum ether/ethyl acetate=10/1 to 5/1) to give methyl 2,5-dibromo-3-[2-[tert-butyl(dimethyl)silyl]oxyethoxy]benzoate (12.0 g, 99% yield) as yellow oil. ¹H NMR (CDCl₃, 400 MHz) δ 7.41 (d, J=2.4 Hz, 1H), 7.21 (d, J=2.0 Hz, 1H), 4.14 (t, J=4.8 Hz, 2H), 4.02 (t, J=5.2 Hz, 2H), 3.94 (s, 3H), 0.91 (s, 9H), 0.11 (s, 6H).

### 4.6 Preparation of methyl 2-bromo-5-(tert-butoxycarbonylamino)-3-[2-[tert-butyl(dimethyl) silyl]oxyethoxy]benzoate

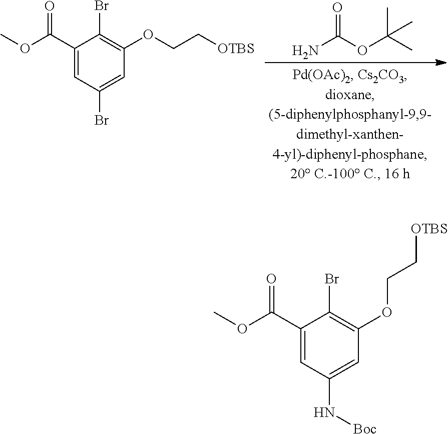

To a mixture of methyl 2,5-dibromo-3-[2-[tert-butyl(dimethyl)silyl]oxyethoxy]benzoate (5.0 g, 10.6 mmol, 1 eq) and NH₂Boc (1.38 g, 11.7 mmol, 1.1 eq) in dioxane (80 mL) was added Pd(OAc)₂ (239 mg, 1.07 mmol, 0.1 eq), (5-diphenylphosphanyl-9,9-dimethyl-xanthen-4-yl)-diphenyl-phosphane (617 mg, 1.07 mmol, 0.1 eq) and Cs₂CO₃ (6.96 g, 21.36 mmol, 2 eq) in one portion at 20° C. under N₂. The mixture was heated to 100° C. and stirred for 16 h. TLC showed the reaction was complete. The reaction mixture was filtered and concentrated in vacuum to give a residue. The residue was purified by column chromatography (SiO₂, Petroleum ether/ethyl acetate=10/1 to 5/1) to give methyl 2-bromo-5-(tert-butoxycarbonylamino)-3-[2-[tert-butyl(dimethyl)silyl]oxyethoxy]benzoate (3.3 g, 61% yield) as a white solid. ¹H NMR (CDCl₃, 400 MHz) δ 7.40 (s, 1H), 7.14 (d, J=2.4 Hz, 1H), 6.53 (s, 1H), 4.13 (t, J=4.8 Hz, 2H), 4.02 (t, J=5.2 Hz, 2H), 3.92 (s, 3H), 1.52 (s, 9H), 0.91 (s, 9H), 0.12 (s, 6H).

### 4.7 Preparation of tert-butyl N-[4-bromo-3-[2-[tert-butyl(dimethyl)silyl]oxyethoxy]-5-(hydroxymethyl)phenyl]carbamate

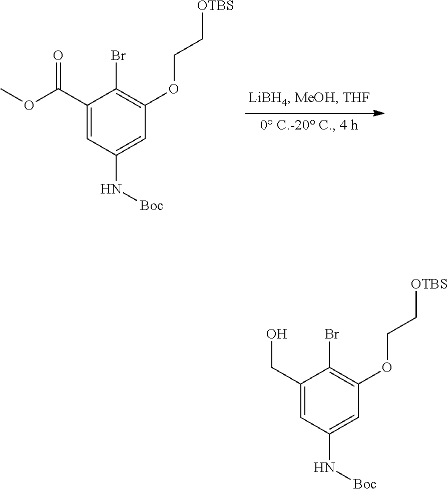 [SMILES: COC(=O)c1cc(NC(=O)OC(C)(C)C)cc(OCCO[Si](C)(C)C(C)(C)C)c1Br ; CC(C)(C)OC(=O)Nc1cc(CO)c(Br)c(OCCO[Si](C)(C)C(C)(C)C)c1]

To a mixture of methyl 2-bromo-5-(tert-butoxycarbonylamino)-3-[2-[tert-butyl (dimethyl) silyl]oxyethoxy]benzoate (4.0 g, 7.93 mmol, 1 eq) in THF (40 mL) was added MeOH (49.4 mmol, 2.00 mL, 6.23 eq) and LiBH₄ (2 M in THF, 15 mL, 3.78 eq) drop-wise at 0° C. under N₂. After the addition, the reaction was allowed to warm to 20° C. and stirred for 4 h. TLC showed the reaction was complete. The mixture was poured into ice-water (100 mL) and the aqueous phase was adjusted pH to 7-8 with aq. HCl (2N). The aqueous phase was extracted with ethyl acetate (50 mL×3). The combined organic phase was washed with brine (50 mL), dried with anhydrous Na₂SO₄, filtered, and concentrated in vacuum to give a residue. The residue was purified by column chromatography (SiO₂, Petroleum ether/ethyl acetate=5/1 to 3/1) to give tert-butyl N-[4-bromo-3-[2-[tert-butyl(dimethyl)silyl]oxyethoxy]-5-(hydroxymethyl) phenyl]carbamate (4 g, crude) as a white solid. ¹H NMR (CDCl₃, 400 MHz) δ 7.26 (s, 1H), 6.91 (d, J=2.4 Hz, 1H), 6.56 (s, 1H), 4.70 (s, 2H), 4.12 (t, J=4.8 Hz, 2H), 4.01 (t, J=4.8 Hz, 2H), 1.52 (s, 9H), 0.92 (s, 9H), 0.12 (s, 6H).

### 4.8 Preparation of tert-butyl N-[7-[2-[tert-butyl(dimethyl)silyl]oxyethoxy]-1-hydroxy-3H-2,1-benzoxaborol-5-yl]carbamate

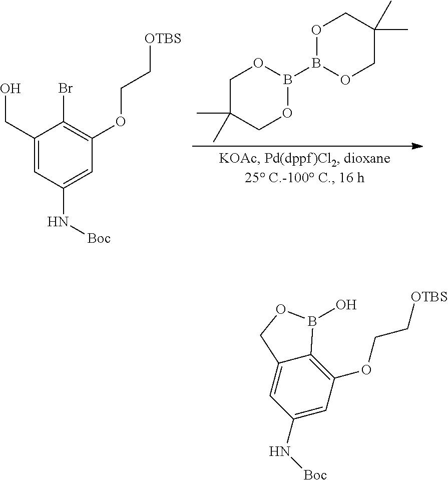

To a mixture of tert-butyl N-[4-bromo-3-[2-[tert-butyl(dimethyl)silyl]oxyethoxy]-5-(hydroxymethyl)phenyl]carbamate (4.0 g, 8.39 mmol, 1 eq) in dioxane (120 mL) was added KOAc (2.47 g, 25.2 mmol, 3 eq), Pd(dppf)Cl₂ (614 mg, 840 umol, 0.1 eq) and 2-(5,5-dimethyl-1,3,2-dioxaborinan-2-yl)-5,5-dimethyl-1,3,2-dioxaborinane (4.74 g, 20.9 mmol, 2.5 eq) in one portion at 25° C. under N₂. Then the mixture was heated to 100° C. and stirred for 16 h. TLC showed the reaction was complete. The reaction mixture was filtered and concentrated in vacuum to give a residue. The residue was purified by column chromatography (SiO₂, Petroleum ether/ethyl acetate=3/1 to 2/1) to give tert-butyl N-[7-[2-[tert-butyl(dimethyl)silyl]oxyethoxy]-1-hydroxy-3H-2,1-benzoxaborol-5-yl]carbamate (2.5 g, crude) as a white solid. ¹H NMR (DMSO-d₆, 400 MHz) δ 7.04 (s, 1H), 6.90 (s, 1H), 6.60 (s, 1H), 5.85 (s, 1H), 5.31 (s, 1H), 5.00 (s, 2H), 4.14 (t, J=4.4 Hz, 2H), 3.98 (t, J=4.8 Hz, 2H), 1.53 (s, 9H), 0.93 (s, 9H), 0.13 (s, 6H).

### 4.9 Preparation of tert-butyl (7,8-dihydro-2H-1,6,9-trioxa-9a-borabenzo[cd]azulen-4-yl)carbamate

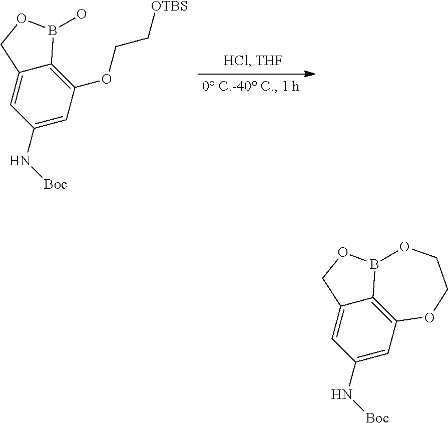 [SMILES: CC(C)(C)OC(=O)Nc1cc2c(c(OCCO[Si](C)(C)C(C)(C)C)c1)B(O)OC2 ; CC(C)(C)OC(=O)Nc1cc2c3c(c1)OCCOB3OC2]

To a mixture of tert-butyl N-[7-[2-[tert-butyl(dimethyl)silyl]oxyethoxy]-1-hydroxy-3H-2,1-benzoxaborol-5-yl]carbamate (250 mg, 590 umol, 1 eq) in THE (10 mL) was added aq. HCl (3 M, 1.18 mL, 6 eq) drop-wise at 0° C. After the addition, the reaction was heated and stirred at 40° C. for 30 min. TLC showed the reaction was complete. The mixture was poured into ice-water (6 mL). The aqueous phase was extracted with ethyl acetate (5 mL×3). The combined organic phase was washed with brine (5 mL), dried with anhydrous Na₂SO₄, filtered, and concentrated in vacuum to give a residue. The residue was purified by prep-TLC (SiO₂, Petroleum ether/ethyl acetate=2:1) and further purified by prep-HPLC (column: Waters Xbridge BEH C18 100*30 mm*10 um; mobile phase: [water (10 mM NH₄HCO₃)-ACN]; B %: 30%-60%, 10 min) to give tert-butyl (7,8-dihydro-2H-1,6,9-trioxa-9a-borabenzo[cd]azulen-4-yl)carbamate (43.2 mg, 99.58% purity) as a off-white solid. ¹H NMR (DMSO-d₆, 400 MHz) δ 9.57 (s, 1H), 7.22 (s, 1H), 6.95 (s, 1H), 5.05 (s, 2H), 4.63-4.59 (m, 1H), 4.30-4.29 (m, 1H), 4.26-4.12 (m, 2H), 1.47 (s, 9H). MS (ESI): mass calcd. For C₁₄H₁₈BNO₅ 291.13, m/z found 290.2[M−H]⁻. HPLC: 99.58% (220 nm), 99.68% (254 nm).

### 4.10 Preparation of 7,8-dihydro-2H-1,6,9-trioxa-9a-borabenzo[cd]azulen-4-amine hydrochloride

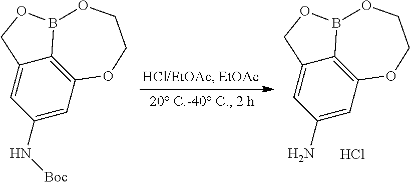 [SMILES: CC(C)(C)OC(=O)Nc1cc2c3c(c1)OCCOB3OC2 ; Nc1cc2c3c(c1)OCCOB3OC2 ; Cl]

To a mixture of tert-butyl (7,8-dihydro-2H-1,6,9-trioxa-9a-borabenzo[cd]azulen-4-yl)carbamate (1.0 g, 3.44 mmol, 1 eq) in EtOAc (10 mL) was added HCl/EtOAc (4 M, 40 mL, 46.58 eq) drop-wise at 20° C. After the addition, the reaction was heated and stirred at 40° C. for 2 h. Then, the mixture was cooled to 10° C. and stirred, solid was precipitate out. The solid was filtered, and filter cake was washed with ethyl acetate (10 mL×2) and dried in vacuuo to give crude product. The crude product was triturated with acetonitrile (10 mL) at 20° C. for 10 min to give 7,8-dihydro-2H-1,6,9-trioxa-9a-borabenzo[cd]azulen-4-amine hydrochloride (550 mg, 96.86% purity) as a white solid. ¹H NMR (DMSO-d₆, 400 MHz) δ 8.60 (s, 3H), 6.63 (s, 1H), 6.43 (s, 1H), 5.04 (s, 2H), 4.63-4.60 (m, 1H), 4.30-4.25 (m, 1H), 4.21-4.15 (m, 2H). MS (ESI): mass calcd. For C₉H₁₁BClNO₃ 227.05, m/z found 192.2 [M+H]⁺. HPLC: 96.86% (220 nm), 98.77% (254 nm).

### 4.11 Preparation of (trans)-2-((2-((7,8-dihydro-2H-1,6,9-trioxa-9a-borabenzo [cd]azulen-4-yl)amino)-5-methylpyrimidin-4-yl)amino)cyclopentanecarbonitrile

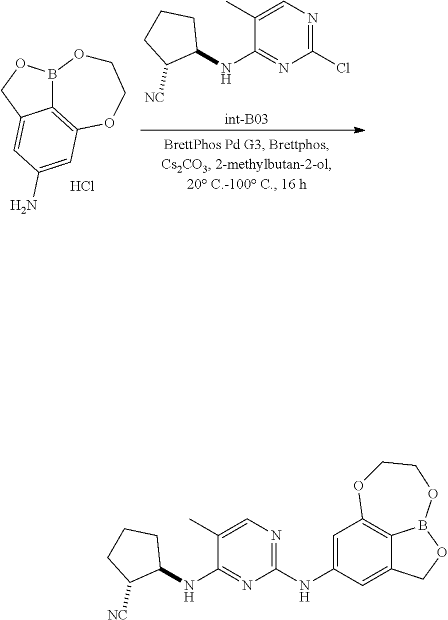

To a mixture of 7,8-dihydro-2H-1,6,9-trioxa-9a-borabenzo[cd]azulen-4-amine hydrochloride (50 mg, 219 umol, 1 eq, HCl salt) and (trans)-2-((2-chloro-5-methylpyrimidin-4-yl)amino) cyclopentanecarbonitrile (57.2 mg, 241 umol, 1.1 eq) in 2-methylbutan-2-ol (2 mL) was added BrettPhos Pd G3 (19.9 mg, 21.9 umol, 0.1 eq), Brettphos (11.8 mg, 21.9 umol, 0.1 eq) and Cs₂CO₃ (214 mg, 659 umol, 3 eq) in one portion at 20° C. under N₂. The mixture was heated to 100° C. and stirred for 16 h. LCMS showed the reaction was complete and desired MS observed. The reaction mixture was filtered and the filtrate was concentrated in vacuum to give a residue. The residue was purified by prep-HPLC (column: Waters Xbridge BEH C18 100*30 mm*10 um; mobile phase: [water (10 mM NH₄HCO₃)-ACN]; B %: 25%-55%, 10 min) to give (trans)-2-((2-((7,8-dihydro-2H-1,6,9-trioxa-9a-borabenzo[cd]azulen-4-yl)amino)-5-methylpyrimidin-4-yl)amino)cyclopentanecarbonitrile (63 mg, 99.18% purity) as a white solid. ¹H NMR (DMSO-d₆, 400 MHz) δ 9.19-9.16 (m, 1H), 7.76 (d, J=3.2 Hz, 1H), 7.53 (d, J=46 Hz, 1H) 7.29 (d, J=25.2 Hz, 1H), 6.85-6.75 (m, 1H), 5.09 (s, 2H), 4.79-4.47 (m, 2H), 4.30-4.25 (m, 2H), 4.16-4.12 (m, 1H), 3.63-3.10 (m, 1H), 2.17-2.05 (m, 2H), 2.01-1.84 (m, 5H), 1.64-1.61 (m, 2H). MS (ESI): mass calcd. For C₂₀H₂₂BN₅O₃ 391.18, m/z found 392.0 [M+H]⁺. HPLC: 99.18% (220 nm), 98.79% (254 nm).

### 5. Preparation of (trans)-2-[[5-chloro-2-[(1-hydroxy-3H-2,1-benzoxaborol-5-yl) amino]pyrimidin-4-yl]amino]cyclopentane-1-carbonitrile

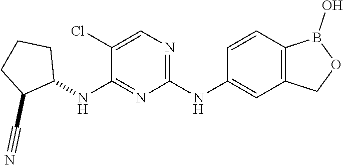

### 5.1 Preparation of methyl 2-bromo-5-[[5-chloro-4-[(trans)(2-cyanocyclopentyl)amino] pyrimidin-2-yl]amino]benzoate

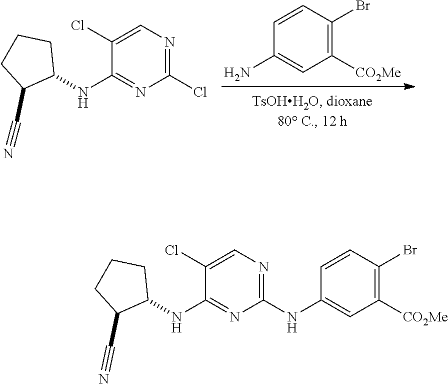

A mixture of (trans)-2-[(2,5-dichloropyrimidin-4-yl)amino]cyclopentane-1-carbonitrile (2.20 g, 8.56 mmol, 1 eq), methyl 5-amino-2-bromo-benzoate (2.17 g, 9.41 mmol, 1.1 eq) and TsOH·H₂O (3.26 g, 17.1 mmol, 2 eq) in dioxane (60 mL) was heated and stirred at 80° C. for 12 h. TLC showed the reaction was complete. The reaction mixture was cooled to room temperature and concentrated in vacuum to give a residue. The residue was triturated with a mixture solvent of EtOAc (50 mL) and saturated aq. Na₂CO₃ (50 mL) and the resulting suspension was filtered. The filter cake was dried in vacuum to give methyl 2-bromo-5-[[5-chloro-4-[(trans)-(2-cyano cyclopentyl)amino]pyrimidin-2-yl]amino]benzoate (2.05 g, 64.8% yield) as a gray solid.

### 5.2 Preparation of methyl 5-[[5-chloro-4-[(trans)-(2-cyanocyclopentyl)amino]pyrimidin-2-yl]amino]-2-(5,5-dimethyl-1,3,2-dioxaborinan-2-yl)benzoate

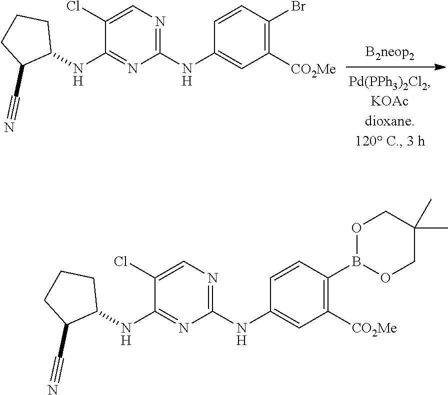

A mixture of methyl 2-bromo-5-[[5-chloro-4-[(2-cyanocyclopentyl)amino]pyrimidin-2-yl] amino]benzoate (2.00 g, 4.44 mmol, 1 eq), 2-(5,5-dimethyl-1,3,2-dioxaborinan-2-yl)-5,5-dimethyl-1,3,2-dioxaborinane (2.51 g, 11.0 mmol, 2.5 eq), Pd(PPh₃)₂Cl₂ (311 mg, 443 umol, 0.1 eq) and KOAc (1.09 g, 11.0 mmol, 2.5 eq) in dioxane (60 mL) was heated and stirred at 120° C. for 3 h. TLC showed the reaction was complete. The reaction mixture was concentrated in vacuum to give a residue. The residue was triturated with a mixture solvent of MTBE (50 mL), EtOAc (5 mL) and petroleum ether (10 mL) to give methyl 5-[[5-chloro-4-[(trans)-(2-cyanocyclopentyl)amino]pyrimidin-2-yl]amino]-2-(5,5-dimethyl-1,3,2-dioxaborinan-2-yl)benzoate (2.0 g, crude) as a brown solid. ¹H NMR (CDCl₃, 400 MHz) δ 8.16 (s, 1H), 8.08 (s, 1H), 7.89 (s, 1H), 7.71 (d, J=7.6 Hz, 2H), 7.50-7.48 (m, 1H), 5.26-5.24 (m, 1H), 4.73-4.66 (m, 1H), 3.90 (s, 3H), 3.77 (s, 4H), 3.57-3.56 (m, 1H), 2.88-2.84 (m, 1H), 2.33-2.31 (m, 1H), 2.15-2.13 (m, 1H), 2.05-2.04 (m, 1H), 1.92-1.86 (m, 2H), 1.65-1.61 (m, 1H), 1.08 (s, 6H).

### 5.3 Preparation of (trans)-2-[[5-chloro-2-[(1-hydroxy-3H-2,1-benzoxaborol-5-yl) amino]pyrimidin-4-yl]amino]cyclopentanecarbonitrile

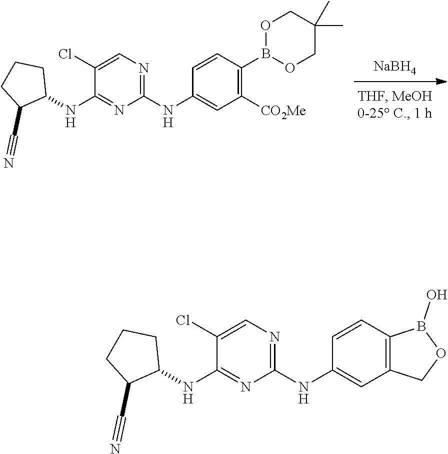

To a solution of methyl To a solution of methyl 5-[[5-chloro-4-[(trans)-2-cyanocyclopentyl) amino]pyrimidin-2-yl]amino]-2-(5,5-dimethyl-1,3,2-dioxaborinan-2-yl)benzoate (5.00 g, 10.3 mmol, 1 eq) in THF (100 mL) and MeOH (5 mL) was added NaBH₄ (1.96 g, 51.6 mmol, 5 eq) portion-wise at 0° C. After the addition, the reaction was allowed to warm to 25° C. and stirred at 25° C. for 1 h. LCMS showed the reaction was complete and desired MS observed. The reaction mixture was quenched with aq. HCl (1 N, 5 mL) carefully and diluted with H₂O (20 mL). The mixture was extracted with EtOAc (50 mL×2). The combined organics were washed with brine, dried over Na₂SO₄, filtered, and concentrated in vacuum to give a residue. The residue was triturated with a mixture solvent of MeCN (10 mL) and H₂O (3 mL) to give (trans)-2-[[5-chloro-2-[(1-hydroxy-3H-2,1-benzoxaborol-5-yl)amino]pyrimidin-4-yl]amino]cyclopentanecarbonitrile (118.6 mg) as a white solid. ¹H NMR (DMSO-d₆, 400 MHz) δ 9.57 (s, 1H), 9.07 (s, 1H), 8.06 (s, 1H), 7.92 (s, 1H), 7.62-7.54 (m, 2H), 7.48-7.46 (m, 1H), 4.95 (s, 2H), 4.83-4.75 (m, 1H), 3.26-3.20 (m, 1H), 2.19-2.09 (m, 2H), 1.89-1.80 (m, 3H), 1.64-1.54 (m, 1H). MS (ESI): mass calcd. For C17H17BClN5O2 369.11, m/z found 370.1 [M+H]⁺. HPLC: 92.18% (220 nm), 93.35% (254 nm).

### 6. Preparation of (trans)2-[[2-[[1-hydroxy-7-(trifluoromethyl)-3H-2,1-benzoxaborol-5-yl]amino]-5-methyl-pyrimidin-4-yl]amino]cyclopentanecarbonitrile

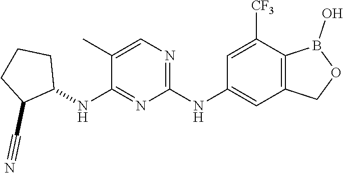

### 6.1 Preparation of methyl 2-bromo-5-[[4-[(2-cyanocyclopentyl)amino]-5-methyl-pyrimidin-2-yl]amino]-3-(trifluoromethyl)benzoate

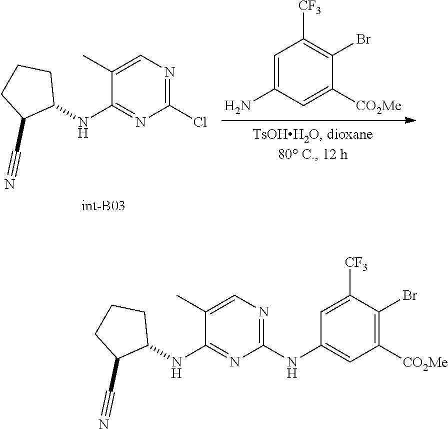

To a mixture of 2-[(2-chloro-5-methyl-pyrimidin-4-yl)amino]cyclopentanecarbonitrile (2.00 g, 8.45 mmol, 1 eq) and methyl 5-amino-2-bromo-3-(trifluoromethyl)benzoate (2.77 g, 9.29 mmol, 1.1 eq) in dioxane (20 mL) was added TsOH·H₂O (3.21 g, 16.9 mmol, 2 eq) in one portion at 20° C. The mixture was heated and stirred at 80° C. for 12 h. TLC showed the reaction was complete. The reaction mixture was adjusted pH=9 by saturated aq. Na₂CO₃. The mixture was extracted with EtOAc (40 mL×3). The combined organic layers were washed with brine (40 mL×2), dried over anhydrous Na₂SO₄, filtered, and concentrated under reduced pressure to give a residue. The residue was triturated with MTBE (30 mL) at 20° C. for 20 min to give methyl 2-bromo-5-[[4-[(2-cyanocyclopentyl) amino]-5-methyl-pyrimidin-2-yl]amino]-3-(trifluoromethyl) benzoate (1.1 g, 26.1% yield) as a white solid. ¹H NMR (CDCl₃, 400 MHz) δ 11.16 (s, 1H), 8.62-8.60 (m, 1H), 8.30 (s, 1H), 8.16 (s, 1H), 7.89 (s, 1H), 4.75-4.67 (m, 1H), 3.91 (s, 3H), 3.30-3.26 (m, 1H), 2.27-2.23 (m, 1H), 2.06-2.03 (m, 4H), 1.80-1.79 (m, 2H), 1.72-1.68 (m, 2H).

### 6.2 Preparation of 2-[[2-[4-bromo-3-(hydroxymethyl)-5-(trifluoromethyl)anilino]-5-methylpyrimidin-4-yl]amino]cyclopentanecarbonitrile

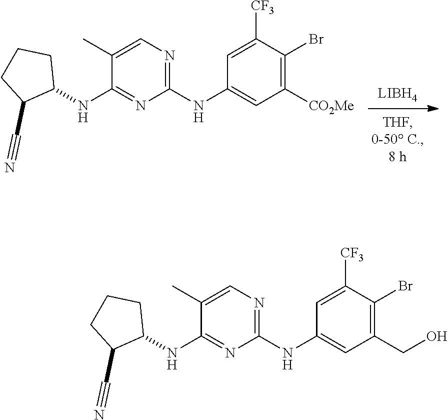

To a mixture of methyl 2-bromo-5-[[4-[(2-cyanocyclopentyl)amino]-5-methyl-pyrimidin-2-yl] amino]-3-(trifluoromethyl)benzoate (0.65 g, 1.30 mmol, 1 eq) in THF (15 mL) was dropwise added LiBH₄ (4 M in THF, 1.63 mL, 5 eq) at 0° C. The mixture was heated and stirred at 50° C. for 8 h. TLC showed the reaction was complete. The reaction was quenched with ice water (30 mL), and adjusted pH=2 by aq. HCl (2N). The white solid was precipitated and filtered. The filter cake was washed with H₂O (10 mL) to give 2-[[2-[4-bromo-3-(hydroxymethyl)-5-(trifluoromethyl)anilino]-5-methyl-pyrimidin-4-yl]amino]cyclopentane carbonitrile (0.45 g, crude) as a white solid. ¹H NMR (CDCl₃, 400 MHz) δ 8.54 (s, 1H), 8.21 (s, 1H), 8.13 (s, 1H), 7.82 (s, 1H), 7.75-7.73 (m, 1H), 5.68-5.65 (m, 1H), 4.81-4.72 (m, 1H), 4.60-4.54 (m, 2H), 3.32 (s, 3H), 3.13-3.08 (m, 1H), 2.19-2.14 (m, 2H), 2.00 (s, 3H), 1.73-1.72 (m, 1H), 1.71-1.65 (m, 2H), 1.58-1.55 (m, 1H).

### 6.3 Preparation of (trans)-2-[[2-[[1-hydroxy-7-(trifluoromethyl)-3H-2,1-benzoxaborol-5-yl]amino]-5-methyl-pyrimidin-4-yl]amino]cyclopentanecarbonitrile

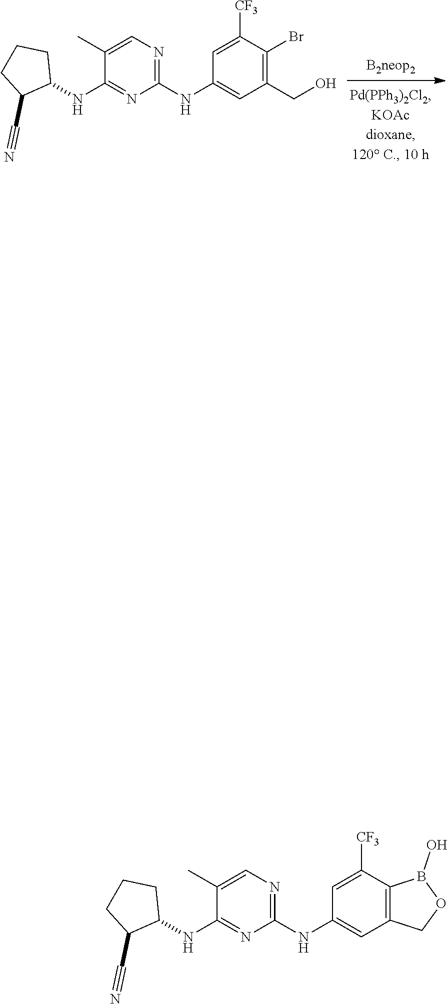

Five reactions were carried out in parallel. For each a mixture of 2-[[2-[4-bromo-3-(hydroxymethyl)-5-(trifluoromethyl)anilino]-5-methyl-pyrimidin-4-yl]amino]cyclopentanecarbonitrile (60 mg, 127 umol, 1 eq) and 2-(5,5-dimethyl-1,3,2-dioxaborinan-2-yl)-5,5-dimethyl-1,3,2-dioxaborinane (72 mg, 318 umol, 2.5 eq) in dioxane (2 mL) was added KOAc (31 mg, 318 umol, 2.5 eq) and Pd(PPh₃)₂Cl₂ (8.95 mg, 12.7 umol, 0.1 eq) in one portion at 20° C. under N₂. The mixture was heated and stirred at 120° C. for 10 h. LCMS showed the reaction was complete and desired MS observed. The five parallel reactions were combined and the mixture was filtered and concentrated under reduced pressure to give a residue. The residue was purified by prep-HPLC (column: Welch Xtimate C18 100*25 mm*3 um; mobile phase: [water (0.04% HCl)-ACN]; B %: 20%-30%, 8 min) to give 2-[[2-[[1-hydroxy-7-(trifluoromethyl)-3H-2,1-benzoxaborol-5-yl]amino]-5-methyl-pyrimidin-4-yl]amino]cyclopentanecarbonitrile (77.1 mg, 30.2% yield) a white solid. ¹H NMR (DMSO-d₆, 400 MHz) δ 10.61 (s, 1H), 9.09 (s, 1H), 8.10 (m, 2H), 7.86 (s, 1H), 5.06 (s, 2H), 4.79-4.75 (m, 1H), 3.25-3.13 (m, 1H), 2.22-2.21 (m, 1H), 2.05-2.03 (m, 4H), 1.82-1.65 (m, 4H). MS (ESI): mass calcd. For Cis 115B04, 417.16, m/z found 418.2 [M+H]⁺. HPLC: 99.09% (220 nm), 99.29 (254 nm).

### 7. Preparation of (trans)-2-((2-((2-hydroxy-2H-benzo[e][1,2]oxaborinin-6-yl)amino)-5-methylpyrimidin-4-yl)amino)cyclopentane-1-carbonitrile

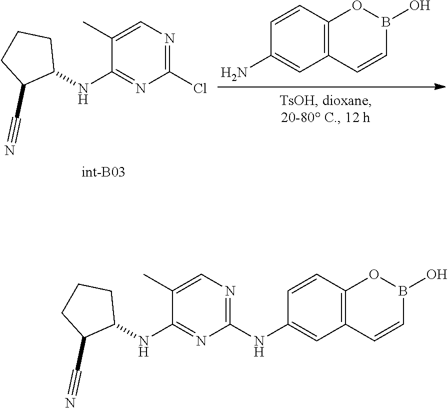

To a mixture of (trans)-2-[(2-chloro-5-methyl-pyrimidin-4-yl)amino]cyclopentanecarbonitrile (100 mg, 422 umol, 1 eq) and 2-hydroxy-1,2-benzoxaborinin-6-amine (83.4 mg, 422 umol, 1 eq, HCl) in dioxane (5 mL) was added TsOH·H₂O (121 mg, 634 umol, 1.5 eq) at 20° C., the mixture was stirred at 80° C. for 12 h. TLC showed the reaction was complete. The reaction mixture was concentrated under reduced pressure to give a residue. The residue was purified by prep-HPLC (column: Waters Xbridge BEH C18 100*30 mm*10 um; mobile phase: [water (10 mM NH₄HCO₃)-ACN]; B %: 15%-45%, 10 min) to give (trans)-2-((2-((2-hydroxy-2H-benzo[e][1,2]oxaborinin-6-yl)amino)-5-methylpyrimidin-4-yl)amino)cyclopentane-1-carbonitrile (17 mg, 95.13% purity) as a yellow solid. ¹H NMR (DMSO-d₆, 400 MHz) δ 9.03 (s, 1H), 8.83 (s, 1H), 7.88 (s, 1H), 7.76-7.68 (m, 2H), 7.60 (d, J=7.6 Hz, 1H), 7.13 (d, J=8.8 Hz, 1H), 6.94-6.70 (m, 1H), 6.11 (d, J=11.6 Hz, 1H), 4.79-4.72 (m, 1H), 3.19-3.09 (m, 1H), 2.20-2.15 (m, 1H), 2.12-2.07 (m, 1H), 1.94 (s, 3H), 1.85-1.77 (m, 2H), 1.73-1.66 (m, 1H), 1.62-1.57 (m, 1H). MS (ESI): mass calcd. For C₁₉H₂₀BN₅O₂ 361.17, m/z found 362.2 [M+H]⁺. HPLC: 95.13% (220 nm), 97.25% (254 nm).

### 8. Preparation of (trans)2-[[2-[(1-hydroxy-3H-2,1-benzoxaborol-5-yl)amino]-5-methyl-pyrimidin-4-yl]amino]cyclohexane-1-carbonitrile

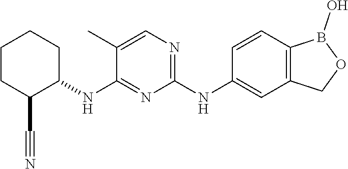

### 8.1 Preparation of methyl 2-bromo-5-[[4-[(trans)-(2-cyanocyclohexyl)amino]-5-methyl-pyrimidin-2-yl]amino]benzoate

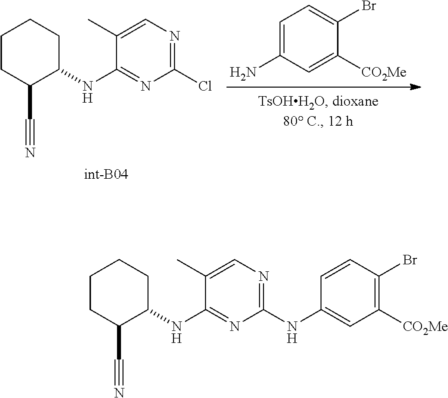

To a mixture of (trans)-2-[(2-chloro-5-methyl-pyrimidin-4-yl)amino]cyclohexanecarbonitrile (1.30 g, 5.18 mmol, 1 eq) and methyl 5-amino-2-bromo-benzoate (1.43 g, 6.22 mmol, 1.2 eq) in dioxane (20 mL) was added TsOH·H₂O (1.97 g, 10.3 mmol, 2 eq) in one portion at 20° C. The mixture was heated and stirred at 80° C. for 12 h. TLC showed the reaction was complete. The reaction mixture was filtered and the filtrate was concentrated under reduced pressure to give a residue. The crude product was triturated with MTBE (10 mL) at 20° C. for 10 min to give methyl 2-bromo-5-[[4-[(2-cyanocyclohexyl) amino]-5-methyl-pyrimidin-2-yl]amino]benzoate (0.8 g, 34% yield) as an off-white solid. ¹H NMR (DMSO-d₆, 400 MHz) δ 9.29 (s, 1H), 8.26 (d, J=2.8 Hz, 1H), 7.76-7.73 (m, 2H), 7.55 (d, J=8.8 Hz, 1H), 6.74 (d, J=8.8 Hz, 1H), 4.27 (br d, J=5.2 Hz, 1H), 3.85 (s, 3H), 3.31 (s, 3H), 2.93 (dt, J=3.6, 11.6 Hz, 1H), 2.21-2.10 (m, 1H), 1.97-1.84 (m, 4H), 1.80-1.52 (m, 3H), 1.38-1.13 (m, 4H).

### 8.2 Preparation of methyl 5-[[4-[((trans)-2-cyanocyclohexyl)amino]-5-methyl pyrimidin-2-yl]amino]-2-(5,5-dimethyl-1,3,2-dioxaborinan-2-yl)benzoate

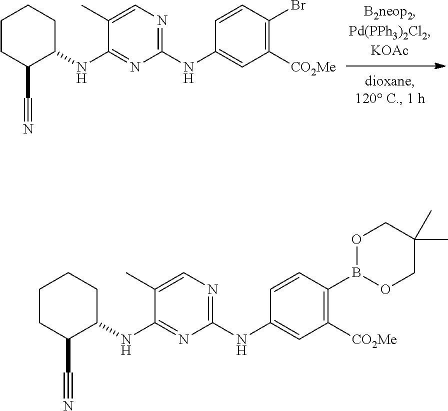

To a mixture of methyl 2-bromo-5-[[4-[((trans)-2-cyanocyclohexyl)amino]-5-methyl-pyrimidin-2-yl]amino] benzoate (800 mg, 1.80 mmol, 1 eq) and 2-(5,5-dimethyl-1,3,2-dioxaborinan-2-yl)-5,5-dimethyl-1,3,2-dioxaborinane (1.02 g, 4.50 mmol, 2.5 eq) in dioxane (10 mL) was added KOAc (442 mg, 4.50 mmol, 2.5 eq) and Pd(PPh₃)₂Cl₂ (253 mg, 360 umol, 0.2 eq) in one portion at 20° C. under N₂. The mixture was heated and stirred at 120° C. for 1 h. TLC showed the reaction was complete. The reaction mixture was filtered and the filtrate was concentrated under reduced pressure to give a residue. The crude product was triturated with MTBE (10 mL) at 20° C. for 15 min to give methyl 5-[[4-[((trans)2-cyanocyclohexyl)amino]-5-methyl-pyrimidin-2-yl]amino]-2-(5,5-dimethyl-1,3,2-dioxaborinan-2-yl)benzoate (0.8 g, crude) as an off-white solid.

### 8.3 Preparation of (trans)-2-[[2-[(1-hydroxy-3H-2,1-benzoxaborol-5-yl)amino]-5-methyl-pyrimidin-4-yl]amino]cyclohexane-1-carbonitrile

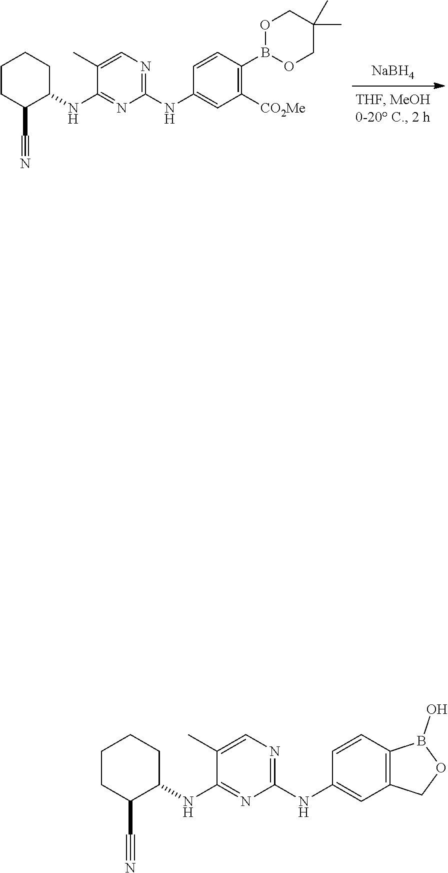

To a mixture of methyl 5-[[4-[((trans)2-cyanocyclohexyl)amino]-5-methyl-pyrimidin-2-yl]amino]-2-(5,5-dimethyl-1,3,2-dioxaborinan-2-yl)benzoate (800 mg, 1.68 mmol, 1 eq) in THF (10 mL) and MeOH (1 mL) was added NaBH₄ (159 mg, 4.19 mmol, 2.5 eq) in portions at 0° C. After the addition, the reaction was allowed to warm to 20° C. and stirred at 20° C. for 2 h. The reaction mixture was adjusted pH=6 by aq. HCl (2N). The mixture was extracted with EtOAc (10 mL×3). The combined organic layers were washed with brine (10 mL×2), dried over anhydrous Na₂SO₄, filtered, and concentrated under reduced pressure to give a residue. The residue was purified by prep-HPLC (column: Phenomenex luna C18 80*40 mm*3 um; mobile phase: [water (0.04% HCl)-ACN]; B %: 18%-43%, 7 min) to give (trans)-2-[[2-[(1-hydroxy-3H-2,1-benzoxaborol-5-yl)amino]-5-methyl-pyrimidin-4-yl]amino]cyclohexanecarbonitrile (80 mg, 13% yield) as a white solid. ¹H NMR (DMSO-d₆, 400 MHz) δ 12.37 (s, 1H), 10.76 (s, 1H), 9.17 (s, 1H), 8.56 (d, J=8.4 Hz, 1H), 7.87 (s, 1H), 7.76-7.72 (m, 2H), 7.48 (dd, J=1.6, 8.0 Hz, 1H), 5.05-4.96 (m, 2H), 4.32-4.23 (m, 1H), 3.08 (m, 1H), 2.19 (d, J=12.8 Hz, 1H), 2.05 (s, 3H), 1.96-1.94 (m, 1H), 1.80-1.71 (m, 2H), 1.66-1.56 (m, 1H), 1.46-1.37 (m, 1H), 1.33-1.15 (m, 2H). MS (ESI): mass calcd. For C₁₉H₂₃BClN₅O₂, 363.19, m/z found 364.2 [M+H]⁺. HPLC: 96.51% (220 nm), 99.68 (254 nm).

### 9. Preparation of (trans)-2-((2-((1-hydroxy-7-methyl-1,3-dihydrobenzo[c][1,2]oxaborol-5-yl)amino)-5-methylpyrimidin-4-yl)amino)cyclohexane-1-carbonitrile

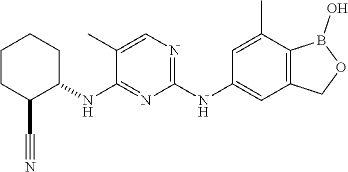

### 9.1 Preparation of 2-bromo-3-methyl-5-nitro-benzoic acid

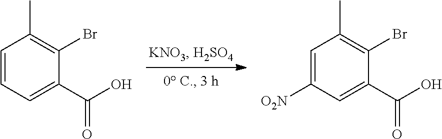

To a mixture of 2-bromo-3-methyl-benzoic acid (10 g, 46.5 mmol, 1 eq) in H₂SO₄ (70 mL, 98% purity) was added a solution of KNO₃ (4.70 g, 46.5 mmol, 1 eq) in H₂SO₄ (30 mL, 98% purity) at 0° C. over 0.5 h, and the mixture was stirred at 0° C. for 2.5 h. TLC showed the reaction was complete. The reaction mixture was poured into ice H₂O (200 mL), the resulting solid was filtered and the filter cake was extracted with ethyl acetate (100 mL). The combined organic phase was washed with brine (150 mL×2), dried with anhydrous Na₂SO₄, filtered, and concentrated in vacuum to give 2-bromo-3-methyl-5-nitro-benzoic acid (10.5 g, 86% yield) as a yellow solid. ¹H NMR (CDCl₃, 400 MHz) δ 8.58 (d, J=2.4 Hz, 1H), 8.28 (d, J=2.4 Hz, 1H), 2.63 (s, 3H).

### 9.2 Preparation of methyl 2-bromo-3-methyl-5-nitro-benzoate

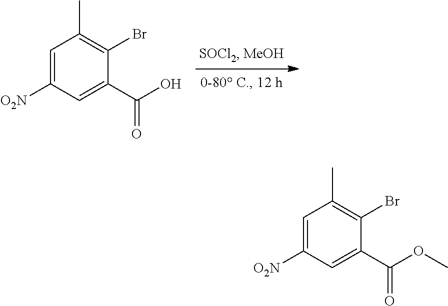

To a mixture of 2-bromo-3-methyl-5-nitro-benzoic acid (8.00 g, 30.8 mmol, 1 eq) in MeOH (80 mL) was added SOCl₂ (61.5 mmol, 4.46 mL, 2 eq) at 0° C. After the addition, the reaction was heated and stirred at 80° C. for 12 h. TLC showed the reaction was complete. The reaction was concentrated under reduced pressure to give a residue. The residue was dissolved in H₂O (80 mL) and adjusted pH to 7 with saturated aq. NaHCO₃ and extracted with EtOAc (30 mL×3). The combined organic layers were washed with brine (50 mL×3), dried over Na₂SO₄, filtered, and concentrated under reduced pressure to give a residue. The residue was purified by flash silica gel chromatography (ISCO®; 80 g SepaFlash® Silica Flash Column, Eluent of 0˜15% Ethyl acetate/petroleum ether gradient @ 100 mL/min) to give methyl 2-bromo-3-methyl-5-nitro-benzoate (6 g, 71% yield) as a yellow solid. ¹H NMR (CDCl₃, 400 MHz) 8.35 (d, J=2.4 Hz, 1H), 8.21 (d, J=2.4 Hz, 1H), 4.00 (s, 3H) 2.59 (s, 3H).

### 9.3 Preparation of methyl 5-amino-2-bromo-3-methyl-benzoate

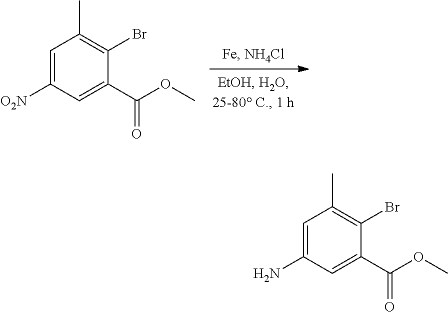

To a mixture of methyl 2-bromo-3-methyl-5-nitro-benzoate (6 g, 21.9 mmol, 1 eq) in EtOH (80 mL) and H₂O (20 mL) was added NH₄Cl (4.68 g, 87.6 mmol, 4 eq) and Fe (4.89 g, 87.6 mmol, 4 eq) at 25° C. under N₂. The mixture was stirred at 80° C. for 1 h. TLC showed the reaction was complete. The reaction was filtered and concentrated under reduced pressure to give a residue. The residue was dissolved in DCM (50 mL), dried over Na₂SO₄, filtered, and concentrated under reduced pressure to give methyl 5-amino-2-bromo-3-methyl-benzoate (5 g, 93% yield) as yellow oil. ¹H NMR (CDCl₃, 400 MHz) δ 6.80 (d, J=2.8 Hz, 1H), 6.68 (d, J=3.2 Hz, 1H), 3.91 (s, 3H), 3.72 (s, 2H), 2.36 (s, 3H).

### 9.4 Preparation of methyl 2-bromo-5-[[4-[((trans)-2-cyanocyclohexyl)amino]-5-methyl-pyrimidin-2-yl]amino]-3-methyl-benzoate

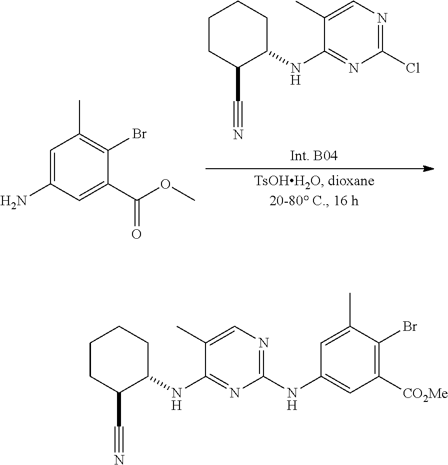

To a mixture of (trans)-2-[(2-chloro-5-methyl-pyrimidin-4-yl)amino]cyclohexane-1-carbonitrile (2.00 g, 7.98 mmol, 1 eq) and methyl 5-amino-2-bromo-3-methyl-benzoate (2.14 g, 8.77 mmol, 1.1 eq) in dioxane (40 mL) was added 4-methylbenzenesulfonicacid·hydrate (3.03 g, 16.0 mmol, 2 eq) in one portion at 20° C. under N₂. The mixture was heated to 80° C. and stirred for 16 h. TLC showed the reaction was complete. The mixture was poured into ice-water (80 mL) and the aqueous phase was adjusted pH to 5-6 with saturated aq. Na₂CO₃. The resulting mixture was diluted with EtOAc (30 mL×3) and then the suspension was filtered. The filter cake was washed with water (30 mL×2) and dried in vacuum to give residue. The residue was purified by trituration with MTBE (30 mL) at 20° C. for 0.5 h to give methyl 2-bromo-5-[[4-[((trans)-2-cyanocyclohexyl)amino]-5-methyl-pyrimidin-2-yl]amino]-3-methyl-benzoate (1.5 g, 41% yield) as an off-white solid.

### 9.5 Preparation of methyl 5-((4-(((trans)-2-cyanocyclohexyl)amino)-5-methylpyrimidin-2-yl)amino)-2-(5,5-dimethyl-1,3,2-dioxaborinan-2-yl)-3-methylbenzoate

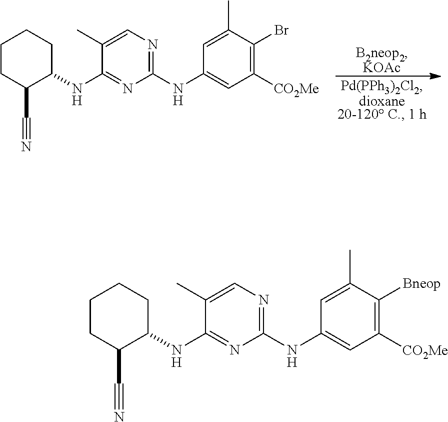

Three reactions were carried out in parallel. For each a mixture of methyl 2-bromo-5-[[4-[((trans)-2-cyanocyclohexyl)amino]-5-methyl-pyrimidin-2-yl]amino]-3-methyl-benzoate (200 mg, 436 umol, 1 eq) and 2-(5,5-dimethyl-1,3,2-dioxaborinan-2-yl)-5,5-dimethyl-1,3,2-dioxaborinane (246 mg, 1.09 mmol, 2.5 eq) in dioxane (6 mL) was added AcOK (107 mg, 1.09 mmol, 2.5 eq) and Pd(PPh₃)₂Cl₂ (15.3 mg, 21.8 umol, 0.05 eq) in one portion at 20° C. under N₂. The mixture was heated and stirred at 120° C. for 1 h. TLC showed the reaction was complete. The three parallel reactions were combined and the reaction mixture was filtered and concentrated in vacuum to give a residue. The crude product was triturated with MTBE (20 mL) at 20° C. for 0.5 h to give methyl 5-((4-(((trans)-2-cyanocyclohexyl)amino)-5-methylpyrimidin-2-yl)amino)-2-(5,5-dimethyl-1,3,2-dioxaborinan-2-yl)-3-methylbenzoate (300 mg, 46% yield) as a gray solid. ¹H NMR (CDCl₃, 400 MHz) δ 7.97 (s, 1H), 7.77 (s, 1H), 7.61 (s, 1H), 4.59-4.48 (m, 1H), 4.44-4.37 (m, 1H), 3.94 (s, 3H), 3.82 (s, 4H), 2.86-2.76 (m, 1H), 2.48 (s, 3H), 2.21-2.18 (m, 1H), 1.99 (s, 3H), 1.84-1.71 (m, 4H), 1.60-1.49 (m, 2H), 1.42-1.36 (m, 2H), 1.15 (s, 6H).

### 9.6 Preparation of (trans)-2-((2-((1-hydroxy-7-methyl-1,3-dihydrobenzo[c][1,2] oxaborol-5-yl)amino)-5-methylpyrimidin-4-yl)amino)cyclohexane-1-carbonitrile

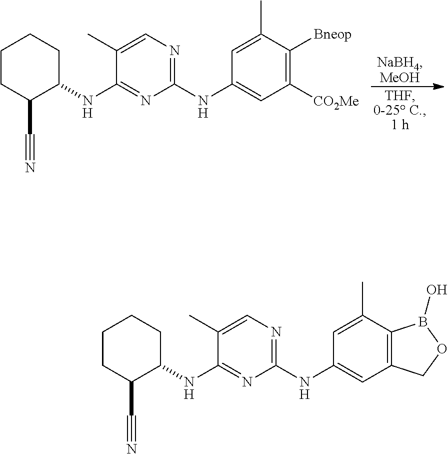

To a solution of methyl methyl 5-((4-(((trans)-2-cyanocyclohexyl)amino)-5-methylpyrimidin-2-yl)amino)-2-(5,5-dimethyl-1,3,2-dioxaborinan-2-yl)-3-methylbenzoate (150 mg, 305 umol, 1 eq) and MeOH (0.5 mL) in THF (6 mL) was added NaBH₄ (57.7 mg, 1.53 mmol, 5 eq) at 0° C. After the addition, the reaction was allowed to warm to 25° C. and stirred for 1 h. The reaction was quenched by H₂O (10 mL), adjusted pH to 5 with aq. HCl (2N). EtOAc (10 mL) was added to the reaction mixture, then precipitate formed. The precipitate was filtered to give (trans)-2-((2-((1-hydroxy-7-methyl-1,3-dihydrobenzo[c][1,2]oxaborol-5-yl)amino)-5-methylpyrimidin-4-yl)amino)cyclohexane-1-carbonitrile (28 mg, 95.47% purity) as an off-white solid. ¹H NMR (DMSO-d₆, 400 MHz) δ 9.03 (s, 1H), 8.57 (s, 1H), 7.74 (s, 1H), 7.71 (s, 1H), 7.37 (s, 1H), 6.68 (d, J=8.8 Hz, 1H), 4.94-4.85 (m, 2H), 4.38-4.25 (m, 1H), 2.99-2.91 (m, 1H), 2.38 (s, 3H), 2.18 (d, J=12.4 Hz, 1H), 1.98-1.96 (m, 1H), 1.94 (s, 3H), 1.81-1.70 (m, 2H), 1.65-1.56 (m, 1H), 1.37-1.30 (m, 2H), 1.24-1.15 (m, 1H). MS (ESI): mass calcd. For C₂₀H₂₄BN₅O₂ 377.20, m/z found 378.2 [M+H]⁺. HPLC: 95.47% (220 nm), 94.36% (254 nm).

### 10. Preparation of (trans)-2-[[2-[(7-chloro-1-hydroxy-1,3dihydrobenzo[c][1,2]oxaboroll-5-yl)amino]-5-methyl-pyrimidin-4-yl]amino]cyclohexane-1-carbonitrile

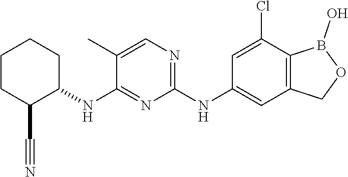

### 10.1 Preparation of (trans)-2-[[2-[4-bromo-3-chloro-5-(hydroxymethyl)anilino]-5-methyl pyrimidin-4-yl]amino]cyclohexane-1-carbonitrile

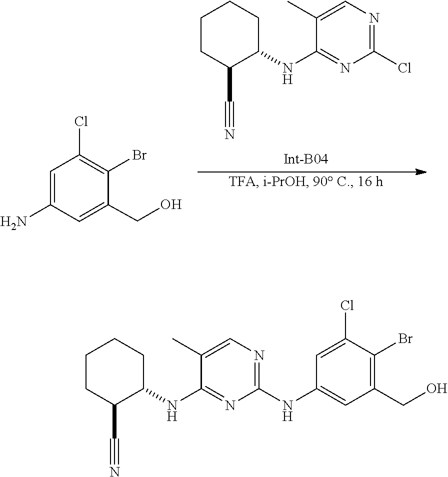

To a solution of (5-amino-2-bromo-3-chloro-phenyl)methanol (3 g, 12.6 mmol, 1 eq) in i-PrOH (30 mL) was added TFA (2.89 g, 25.37 mmol, 1.90 mL, 2 eq) and 2-((2-chloro-5-methylpyrimidin-4-yl)amino)cyclohexane-1-carbonitrile (3.50 g, 13.95 mmol, 1.1 eq) at 20° C. The mixture was heated to 90° C. and stirred at 90° C. for 16 hrs. Solid precipitated out after 3 hours reaction time. TLC showed the reaction was complete. The reaction was filtered to give the crude product. The crude product was triturated with aqueous NaHCO₃ (40 mL) at 20° C. for 30 min, then filtered. The filtered cake was washed with THE (100 mL) to give (trans)-2-[[2-[4-bromo-3-chloro-5-(hydroxymethyl)anilino]-5-methyl-pyrimidin-4-yl]amino]cyclohexane-1-carbonitrile (5 g, 87% purity) as a white solid. ¹H NMR (400 MHz, DMSO-d₆) δ=8.12-7.89 (m, 2H), 7.85 (s, 1H), 7.73 (s, 1H), 5.69 (s, 1H), 4.52 (s, 2H), 4.41-4.28 (m, 1H), 3.84-3.72 (m, 1H), 3.00 (dt, J=2.4, 12.0 Hz, 1H), 2.16 (d, J=12.4 Hz, 1H), 2.02 (s, 3H), 1.90-1.80 (m, 1H), 1.77-1.52 (m, 3H), 1.50-1.30 (m, 2H), 1.27-1.10 (m, 1H).

### 10.2 Preparation of 2-[[2-[4-bromo-3-[[tert-butyl(dimethyl)silyl]oxymethyl]-5-chloro-anilino]-5-methylpyrimidin-4-yl]amino]cyclohexane-1-carbonitrile

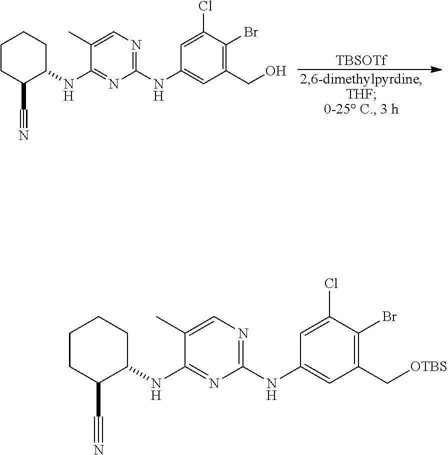

To a solution of (trans)-2-[[2-[4-bromo-3-chloro-5-(hydroxymethyl)anilino]-5-methyl-pyrimidin-4-yl]amino]cyclohexanecarbonitrile (6 g, 13.3 mmol, 1 eq) and 2,6-dimethylpyridine (5.76 g, 53.2 mmol, 4 eq) in THE (60 mL) was added TBSOTf (7.04 g, 26.6 mmol, 6.12 mL, 2 eq) dropwise at 0° C. The mixture was stirred at 25° C. for 3 h. LCMS showed the reaction was complete. The reaction mixture was quenched by addition saturated. aq. NH₄Cl (100 mL) at 0° C. and extracted with Ethyl acetate (50 mL×2). The combined organic layers were washed with brine (50 mL), dried over Na₂SO₄, filtered, and concentrated under reduced pressure to give a residue. The residue was triturated with a solution of petroleum ether (15 mL) and ethyl acetate (15 mL) at 20° C. for 30 min to give (trans)-2-[[2-[4-bromo-3-[[tert-butyl(dimethyl)silyl]oxymethyl]-5-chloro-anilino]-5-methyl-pyrimidin-4-yl]amino]cyclo hexane-1-carbonitrile (5.2 g, 69.1% yield) as a white solid. ¹H NMR (400 MHz, DMSO-d₆) δ=9.45 (s, 1H), 8.32 (s, 1H), 7.75 (s, 1H), 7.66 (s, 1H), 6.77 (d, J=8.0 Hz, 1H), 4.65 (s, 2H), 4.43-4.21 (m, 1H), 2.95-2.85 (m, 1H), 2.27-2.08 (m, 1H), 2.03-1.85 (m, 4H), 1.83-1.67 (m, 2H), 1.65-1.52 (m, 1H), 1.47-1.12 (m, 3H), 0.94 (s, 9H), 0.13 (s, 6H).

### 10.3 Preparation of (trans)-2-[[2-[3-[[tert-butyl(dimethyl)silyl]oxymethyl]-5-chloro-4-(5,5-dimethyl-1,3,2-dioxaborinan-2-yl)anilino]-5-methyl pyrimidin-4-yl]amino]cyclohexane-1-carbonitrile

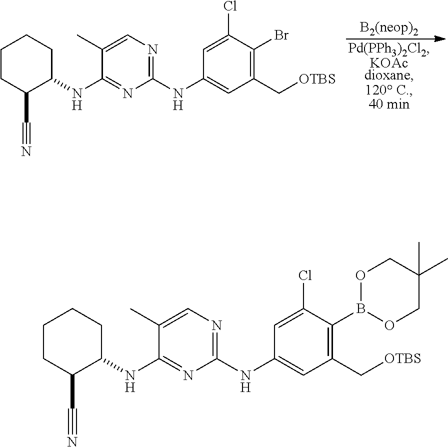

Five reactions were carried out in parallel. For each a solution of (trans)-2-[[2-[4-bromo-3-[[tert-butyl(dimethyl)silyl]oxymethyl]-5-chloro-anilino]-5-methyl-pyrimidin-4-yl]amino]cyclohexane-1-carbonitrile (0.4 g, 708 umol, 1 eq) in dioxane (12 mL) was added 2-(5,5-dimethyl-1,3,2-dioxaborinan-2-yl)-5,5-dimethyl-1,3,2-dioxaborinane (800 mg, 3.54 mmol, 5 eq), KOAC (208 mg, 2.12 mmol, 3 eq) and Pd(PPh₃)₂Cl₂ (50 mg, 70.8 umol, 0.1 eq) at 20° C. Then the mixture was stirred at 120° C. for 40 min under N₂ atmosphere. LCMS showed the reaction was complete. The reactions were cooled to room temperature and the five parallel reactions were combined and filtered. The filtrate was concentrated under reduced pressure to give a residue. The residue was purified by prep-HPLC (column: Welch Xtimate C18 250*70 mm #10 um; mobile phase: [water (NH₄HCO₃)-ACN]; B %: 45%-75%, 20 min) to give 2-[[2-[3-[[tert-butyl(dimethyl) silyl]oxymethyl]-5-chloro-4-(5,5-dimethyl-1,3,2-dioxaborinan-2-yl)anilino]-5-methyl-pyrimidin-4-yl]amino]cyclohexanecarbonitrile (1 g, 47.2% yield, 95% purity) as a white solid. ¹H NMR (400 MHz, DMSO-d₆) δ=9.20 (s, 1H), 7.98 (s, 1H), 7.74 (s, 1H), 7.43 (s, 1H), 6.73 (d, J=8.8 Hz, 1H), 4.63 (s, 2H), 4.42-4.26 (m, 1H), 3.70 (s, 4H), 2.95-2.90 (m, 1H), 2.18 (d, J=11.6 Hz, 1H), 1.98-1.87 (m, 4H), 1.80-1.67 (m, 2H), 1.68-1.53 (m, 1H), 1.46-1.27 (m, 2H), 1.47-1.12 (m, 1H), 1.04 (s, 6H), 0.92-0.87 (m, 9H), 0.06 (s, 6H).

### 10.4 Preparation of (trans)-2-[[2-[(7-chloro-1-hydroxy-3H-2,1-benzoxaborol-5-yl)amino]-5-methyl pyrimidin-4-yl]amino]cyclohexane-1-carbonitrile

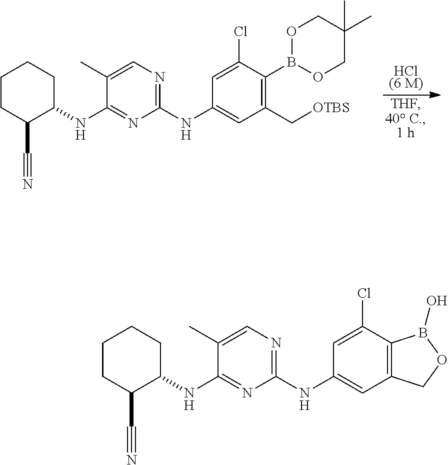

To a solution of (trans)2-[[2-[3-[[tert-butyl(dimethyl)silyl]oxymethyl]-5-chloro-4-(5,5-dimethyl-1,3,2-dioxaborinan-2-yl)anilino]-5-methyl-pyrimidin-4-yl]amino]cyclohexane-1-carbonitrile (1.8 g, 3.40 mmol, 1 eq) in THF (20 mL) was added HCl (6 M, 2.26 mL, 4 eq) at 20° C. The mixture was stirred at 40 for 1 hr. LCMS showed the reaction was complete and one main peak with desired mass was detected. The reaction was concentrated under reduced pressure to give a residue. The residue was triturated with MeCN (15 mL) at 25° C. for 10 min to give racemic product (1.5 g). The racemic product (1.5 g) was purified by SFC (column: DAICEL CHIRALPAK AD (250 mm*30 mm, 10 um); mobile phase: [Neu-IPA]; B %: 42%-42%, 8 min) to give (trans)-2-[[2-[(7-chloro-1-hydroxy-3H-2,1-benzoxaborol-5-yl)amino]-5-methyl-pyrimidin-4-yl]amino]cyclohexane-1-carbonitrile (90.5 mg, 95.79% purity, 98.80% ee, first peak, Rt=1.280 min) stereoisomer one as a white solid. ¹H NMR (400 MHz, DMSO-d₆) δ=9.37 (s, 1H), 8.84 (s, 1H), 7.84 (s, 1H), 7.77 (s, 1H), 7.71 (s, 1H), 6.79 (d, J=8.8 Hz, 1H), 5.00-4.85 (m, 2H), 4.40-4.26 (m, 1H), 2.93 (dt, J=3.2, 11.2 Hz, 1H), 2.19 (d, J=11.2 Hz, 1H), 2.00-1.90 (m, 4H), 1.90-1.76 (m, 2H), 1.75-1.62 (m, 1H), 1.53-1.33 (m, 2H), 1.33-1.19 (m, 1H) MS (ESI): mass calcd. For C₁₉H₂₁BClN₅O₂ 397.15, m/z found 398.0 [M+H]⁺. HPLC: 95.79% (220 nm), 95.88% (254 nm). and (trans)-2-[[2-[(7-chloro-1-hydroxy-3H-2,1-benzoxaborol-5-yl)amino]-5-methyl-pyrimidin-4-yl]amino]cyclohexanecarbonitrile (206 mg, 96.14% purity, 98.78% ee, second peak, Rt=1.841 min) stereoisomer two as a white solid. ¹H NMR (400 MHz, DMSO-d₆) δ=9.37 (s, 1H), 8.84 (s, 1H), 7.84 (s, 1H), 7.77 (s, 1H), 7.71 (s, 1H), 6.78 (d, J=8.8 Hz, 1H), 5.07-4.78 (m, 2H), 4.46-4.13 (m, 1H), 2.93 (dt, J=3.6, 12.0 Hz, 1H), 2.18 (d, J=11.2 Hz, 1H), 2.00-1.90 (s, 4H), 1.83-1.69 (m, 2H), 1.68-1.56 (m, 1H), 1.46-1.27 (m, 2H), 1.26-1.13 (m, 1H). MS (ESI): mass calcd. For C₁₉H₂₁BClN₅O₂ 397.15, m/z found 398.1 [M+H]⁺. HPLC: 96.14% (220 nm), 99.69% (254 nm).

### 11. Preparation of (trans)-2-((2-((1-hydroxy-7-(trifluoromethyl)-1,3-dihydrobenzo[c][1,2]oxaborol-5-yl)amino)-5-methylpyrimidin-4-yl)amino)cyclohexane-1-carbonitrile

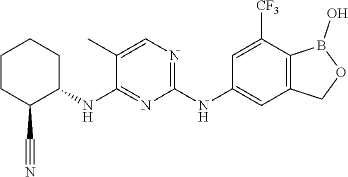 [SMILES: Cc1cnc(Nc2cc3c(c(C(F)(F)F)c2)B(O)OC3)nc1N[C@H]1CCCC[C@@H]1C#N]

### 11.1 Preparation of (trans)-2-[[2-[4-bromo-3-(hydroxymethyl)-5-(trifluoromethyl)anilino]-5-methyl-pyrimidin-4-yl]amino]cyclohexane-1-carbonitrile

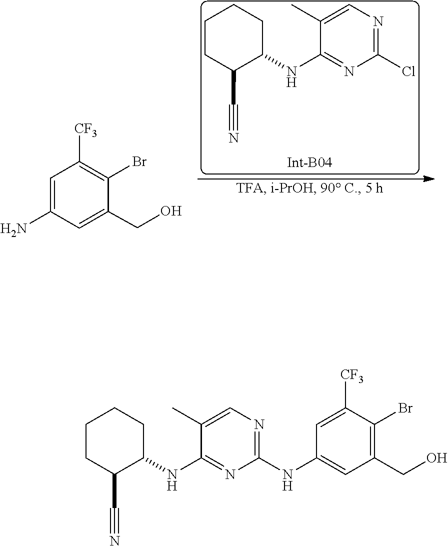

To a mixture of [5-amino-2-bromo-3-(trifluoromethyl)phenyl]methanol (3.6 g, 13.3 mmol, 1 eq) and 2-[(2-chloro-5-methyl-pyrimidin-4-yl)amino]cyclohexanecarbonitrile (3.34 g, 13.3 mmol, 1 eq) in i-PrOH (40 mL) was added TFA (2.28 g, 20.0 mmol, 1.5 mL, 1.5 eq) in one portion at 25° C. The mixture was stirred at 90° C. for 5 h. LCMS showed the reaction was complete and desired MS observed. The reaction mixture was cooled to room temperature and large amount of precipitate formed. The mixture was filtered and the filter cake was triturated with saturated aq. NaHCO₃ to afford 2-[[2-[4-bromo-3-(hydroxymethyl)-5-(trifluoromethyl)anilino]-5-methyl-pyrimidin-4-yl]amino]cyclohexanecarbonitrile (5.2 g, 80.5% yield) as a white solid. ¹H NMR (DMSO-d₆, 400 MHz) δ 9.43 (s, 1H), 8.30 (d, J=2.8 Hz, 1H), 8.18 (d, J=2.4 Hz, 1H), 7.77 (s, 1H), 6.71 (d, J=8.8 Hz, 1H), 5.57 (t, J=5.2 Hz, 1H), 4.54 (d, J=5.2 Hz, 2H), 4.42-4.32 (m, 1H), 2.95-2.88 (m, 1H), 2.18-2.12 (m, 1H), 1.95 (s, 3H), 1.90-1.85 (m, 1H), 1.75-1.65 (m, 2H), 1.66-1.56 (m, 1H), 1.37-1.29 (m, 2H), 1.20-1.10 (m, 1H).

### 11.2 Preparation of (trans)-2-[[2-[4-bromo-3-[[tert-butyl(dimethyl)silyl]oxymethyl]-5-(trifluoromethyl)anilino]-5-methyl pyrimidin-4-yl]amino]cyclohexane-1-carbonitrile

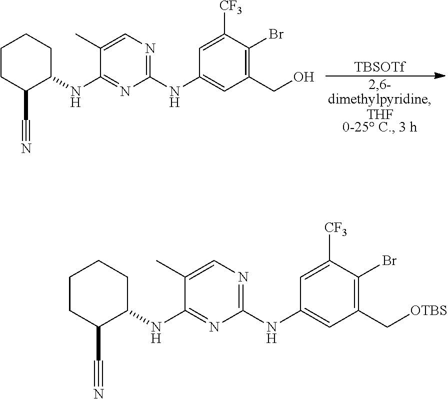

To a solution of (trans)-2-[[2-[4-bromo-3-(hydroxymethyl)-5-(trifluoromethyl)anilino]-5-methyl-pyrimidin-4-yl]amino]cyclohexanecarbonitrile (3.5 g, 7.23 mmol, 1 eq) and 2,6-dimethylpyridine (2.34 g, 21.7 mmol, 2.6 mL, 3 eq) in THF (100 mL) was added TBSOTf (2.87 g, 10.8 mmol, 2.5 mL, 1.5 eq) at 0° C. After the addition, the reaction was allowed to warm to 25° C. and stirred for 3 h. TLC showed the reaction was complete. The reaction mixture was quenched with saturated aq. NH₄Cl (80 mL) at 0° C., and then extracted with EtOAc (50 mL×3). The combined organic layers were washed with brine (50 mL×2), dried over Na₂SO₄, filtered, and concentrated under reduced pressure to give a residue. The residue was triturated with petroleum ether (40 mL) at 25° C. for 30 min to obtain (trans)-2-[[2-[4-bromo-3-[[tert-butyl(dimethyl) silyl]oxymethyl]-5-(trifluoromethyl) anilino]-5-methyl-pyrimidin-4-yl]amino]cyclohexanecarbonitrile (4.1 g, 94.8% yield) as a white solid. ¹H NMR (DMSO-d₆, 400 MHz) δ 9.55 (s, 1H), 8.45 (s, 1H), 8.04 (s, 1H), 7.75 (s, 1H), 6.74 (d, J=9.2 Hz, 1H), 4.72 (s, 2H), 4.37-4.28 (m, 1H), 2.97-2.89 (m, 1H), 2.20-2.14 (m, 1H), 1.95 (s, 3H), 1.88-1.80 (m, 1H), 1.76-1.68 (m, 2H), 1.60-1.50 (m, 1H), 1.39-1.14 (m, 4H), 0.95 (s, 9H), 0.14 (s, 6H).

### 11.3 Preparation of (trans)-2-[[2-[3-[[tert-butyl(dimethyl)silyl]oxymethyl]-4-(5,5-dimethyl-1,3,2-dioxaborinan-2-yl)-5-(trifluoromethyl)anilino]-5-methyl-pyrimidin-4-yl]amino]cyclohexanecarbonitrile

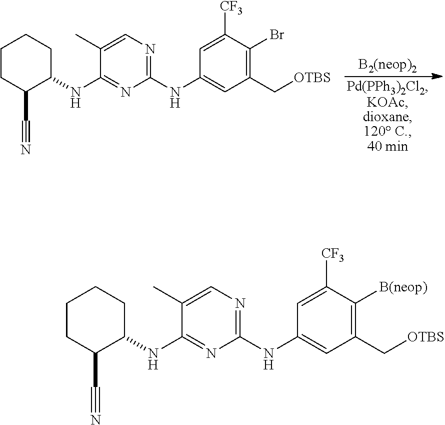

Four reactions were carried out in parallel. For each a mixture of (trans)-2-[[2-[4-bromo-3-[[tert-butyl(dimethyl)silyl]oxymethyl]-5-(trifluoromethyl)anilino]-5-methyl-pyrimidin-4-yl]amino]cyclohexanecarbonitrile (1 g, 1.67 mmol, 1 eq), 2-(5,5-dimethyl-1,3,2-dioxaborinan-2-yl)-5,5-dimethyl-1,3,2-dioxaborinane (1.8 g, 7.97 mmol, 4.8 eq), Pd(PPh₃)₂Cl₂ (117 mg, 167 umol, 0.1 eq), KOAc (492 mg, 5.01 mmol, 3 eq) in dioxane (20 mL) was degassed and purged with N₂ for 3 times, and then the mixture was stirred at 120° C. for 40 min under N₂ atmosphere. LCMS showed the reaction was complete and desired MS observed. The four parallel reactions were combined, filtered and the filter cake was washed with EtOAc (90 mL×2). The filtrate was concentrated under reduced pressure to give a residue (6 g, crude). The residue was purified by flash silica gel chromatography (ISCO®; 120 g SepaFlash® Silica Flash Column, Eluent of 0˜100% Ethyl acetate/petroleum ether gradient @ 75 mL/min) to give crude product, which was purified by prep-HPLC (column: C18 (250*50 mm*10 um); mobile phase: [water (NH₄HCO₃)-ACN]; B %: 75%-95%, 10 min) to give 2-[[2-[3-[[tert-butyl(dimethyl)silyl]oxymethyl]-4-(5,5-dimethyl-1,3,2-dioxaborinan-2-yl)-5-(trifluoromethyl)anilino]-5-methyl-pyrimidin-4-yl]amino]cyclohexanecarbonitrile (2.4 g) as colorless oil. ¹H NMR (DMSO-d₆, 400 MHz) δ 9.34 (s, 1H), 8.27 (s, 1H), 7.79 (s, 1H), 7.75 (s, 1H), 6.69 (d, J=9.2 Hz, 1H), 4.73 (s, 2H), 4.37-4.30 (m, 1H), 3.70 (s, 4H), 2.92 (m, 1H), 2.20-2.15 (m, 1H), 1.95 (s, 3H), 1.90-1.85 (m, 1H), 1.76-1.68 (m, 2H), 1.60-1.50 (m, 1H), 1.36-1.26 (m, 2H), 1.24-1.13 (m, 1H), 1.03 (s, 6H), 0.90 (s, 9H), 0.07 (s, 6H).

### 11.4 Preparation of (trans)-2-((2-((1-hydroxy-7-(trifluoromethyl)-1,3-dihydrobenzo[c][1,2] oxaborol-5-yl)amino)-5-methylpyrimidin-4-yl)amino)cyclohexane-1-carbonitrile

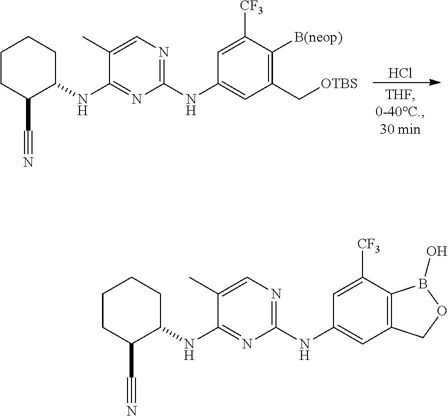

To a mixture of (trans)-2-[[2-[3-[[tert-butyl(dimethyl)silyl]oxymethyl]-4-(5,5-dimethyl-1,3,2-dioxaborinan-2-yl)-5-(trifluoromethyl)anilino]-5-methyl-pyrimidin-4-yl]amino]cyclohexanecarbonitrile (2 g, 3.17 mmol, 1 eq) in THE (20 mL) was added HCl (6 M, 2.64 mL, 5 eq) drop-wise at 0° C. The mixture was stirred at 40° C. for 0.5 h. LCMS showed the reaction was complete and desired MS observed. The reaction mixture was cooled to room temperature and large amount of precipitate formed. The reaction was filtered. The filter cake was washed with deionized water to give crude product. The crude product was triturated with MeCN (20 mL) at 25° C. for 20 min to give (trans)-2-((2-((1-hydroxy-7-(trifluoromethyl)-1,3-dihydrobenzo[c][1,2]oxaborol-5-yl)amino)-5-methylpyrimidin-4-yl)amino)cyclohexane-1-carbonitrile (1.2 g) as a white solid. ¹H NMR (DMSO-d₆, 400 MHz) δ 10.91 (s, 1H), 9.17 (s, 1H), 8.44 (d, J=7.2 Hz, 1H), 8.04 (s, 1H), 7.90 (s, 1H), 7.82 (s, 1H), 5.13-5.04 (m, 2H), 4.32-4.22 (m, 1H), 3.04 (m, 1H), 2.18 (d, J=11.2 Hz, 1H), 2.06 (s, 3H), 1.88 (d, J=12.8 Hz, 1H), 1.78-1.72 (m, 2H), 1.60-1.38 (m, 2H), 1.27-1.16 (m, 2H). Material was separated by prep-SFC (column: DAICEL CHIRALPAK IG (250 mm*50 mm, 10 um); mobile phase: [0.1% NH3H2O MEOH]; B %: 40%-40%, 5 min) to give (trans)-2-((2-((1-hydroxy-7-(trifluoromethyl)-1,3-dihydrobenzo[c][1,2]oxaborol-5-yl)amino)-5-methylpyrimidin-4-yl)amino)cyclohexane-1-carbonitrile (557 mg, 99.90% ee, first peak, Rt=1.246 min) stereoisomer one as an off-white solid. ¹H NMR (DMSO-d₆, 400 MHz) δ 9.51 (s, 1H), 8.89 (s, 1H), 8.14 (s, 1H), 8.04 (s, 1H), 7.79 (s, 1H), 6.76 (d, J=8.8 Hz, 1H), 5.06-4.97 (m, 2H), 4.37-4.28 (m, 1H), 2.93 (m, 1H), 2.18 (d, J=11.6 Hz, 1H), 1.96 (s, 3H), 1.95-1.90 (m, 1H), 1.78-1.70 (m, 2H), 1.65-1.54 (m, 1H), 1.39-1.28 (m, 2H), 1.25-1.14 (m, 1H). MS (ESI): mass calcd. For C₂₀H₂₁BF₃N₅O₂ 431.17, m/z found 432.3 [M+H]⁺. HPLC: 99.25% (220 nm), 99.33% (254 nm) and (trans)-2-((2-((1-hydroxy-7-(trifluoromethyl)-1,3-dihydrobenzo[c][1,2]oxaborol-5-yl)amino)-5-methylpyrimidin-4-yl)amino)cyclohexane-1-carbonitrile (551.7 mg, 99.34% ee, second peak, Rt=1.443 min) stereoisomer two as an off-white solid. ¹H NMR (DMSO-d₆, 400 MHz) δ 9.54 (s, 1H), 8.90 (s, 1H), 8.13 (s, 1H), 8.03 (s, 1H), 7.79 (s, 1H), 6.81 (d, J=8.8 Hz, 1H), 5.06-4.97 (m, 2H), 4.37-4.28 (m, 1H), 2.94 (m, 1H), 2.20-2.15 (m, 1H), 1.97 (s, 3H), 1.95-1.90 (m, 1H), 1.78-1.69 (m, 2H), 1.65-1.54 (m, 1H), 1.39-1.29 (m, 2H), 1.26-1.15 (m, 1H). MS (ESI): mass calcd. For C₂₀H₂₁BF₃N₅O₂ 431.17, m/z found 432.3 [M+H]⁺. HPLC: 97.52% (220 nm), 98.26% (254 nm).

### 12. Preparation of 5-((4-(((trans)-2-cyanocyclohexyl)amino)-5-methylpyrimidin-2-yl)amino)-1-hydroxy-1,3-dihydrobenzo[c][1,2]oxaborole-7-carbonitrile

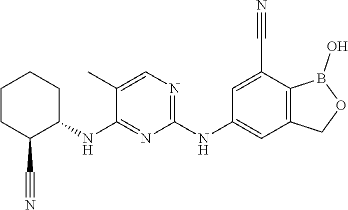 [SMILES: Cc1cnc(Nc2cc(C#N)c3c(c2)COB3O)nc1N[C@H]1CCCC[C@@H]1C#N]

### 12.1 Preparation of methyl 2-amino-3-bromo-5-nitrobenzoate

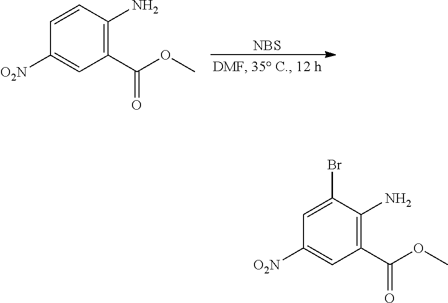

To a solution of methyl 2-amino-5-nitro-benzoate (60 g, 305 mmol, 1 eq) in DMF (700 mL) was added NBS (70.7 g, 397 mmol, 1.3 eq) at 35° C. The mixture was stirred at 35° C. for 12 h. Solid was precipitate out after 3 h reaction. TLC showed the reaction was complete. The reaction mixture was filtered to give crude product (filter cake). The crude product was triturated with MTBE (50 mL) at 25° C. for 1 h to obtain methyl 2-amino-3-bromo-5-nitro-benzoate (70 g, 83.2% yield) as a yellow solid. ¹H NMR (400 MHz, DMSO-d₆) δ=8.61 (d, J=2.8 Hz, 1H), 8.47 (d, J=2.8 Hz, 1H), 3.90 (s, 3H).

### 12.2 Preparation of methyl 2-amino-3-cyano-5-nitrobenzoate

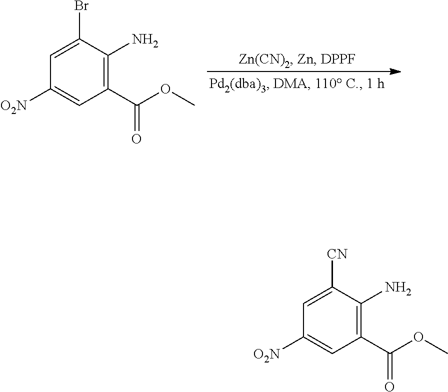

A mixture of methyl 2-amino-3-bromo-5-nitro-benzoate (70 g, 254 mmol, 1 eq), Zn (3.50 g, 53.5 mmol, 0.21 eq), Zn(CN)₂ (59.7 g, 508 mmol, 2 eq), Pd₂(dba)₃ (6.99 g, 7.63 mmol, 0.03 eq) and 1,1-Bis(diphenylphosphino)ferrocene (DPPF, 8.47 g, 15.2 mmol, 0.06 eq) in DMA (600 mL) was degassed and purged with N₂ for 3 times, and then the mixture was stirred at 110° C. for 1 h under N₂ atmosphere. TLC showed the reaction was complete. The reaction mixture was diluted with H₂O (3 L) and extracted with EtOAc (1 L×3). The combined organic layers were washed with brine (500 mL×2), dried over anhydrous Na₂SO₄, filtered, and concentrated under reduced pressure to give a residue. The residue was triturated with DCM (60 mL) at 25° C. for 1 h to obtain methyl 2-amino-3-cyano-5-nitro-benzoate (30 g, 55.3% yield) as a gray solid. ¹H NMR (400 MHz, DMSO-d₆) δ=8.74-8.67 (m, 2H), 8.20 (s, 2H), 3.90 (s, 3H)

### 12.3 Preparation of methyl 2-bromo-3-cyano-5-nitrobenzoate

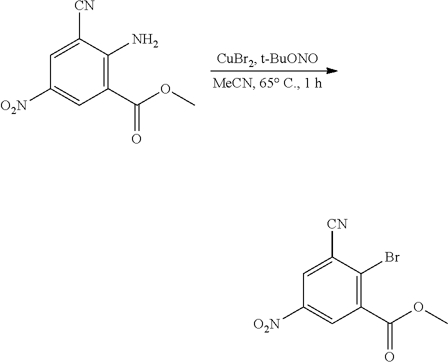

To a mixture of tert-butyl nitrite (27.9 g, 271 mmol, 32.2 mL, 2 eq) and CuBr₂ (45.4 g, 203 mmol, 1.5 eq) in MeCN (100 mL) was added methyl 2-amino-3-cyano-5-nitro-benzoate (30 g, 135 mmol, 1 eq) in portions at 65° C. The mixture was stirred at 65° C. for 1 h. LCMS showed the reaction was complete and desired mass was detected. The reaction mixture was diluted with H₂O (500 mL) and extracted with EtOAc (500 mL×3). The combined organic layers were washed with brine (300 mL×2), dried over Na₂SO₄, filtered, and concentrated under reduced pressure to give a residue. The residue was purified by flash silica gel chromatography (ISCO®; 220 g SepaFlash® Silica Flash Column, eluent of 0˜25% ethyl acetate/petroleum ether gradient @ 100 mL/min) to obtain methyl 2-bromo-3-cyano-5-nitro-benzoate (32 g, 82.7% yield) as a yellow solid. ¹H NMR (400 MHz, DMSO-d₆) δ=8.98 (d, J=2.0 Hz, 1H), 8.76 (d, J=2.0 Hz, 1H), 3.94 (s, 3H).

### 12.4 Preparation of methyl 5-amino-2-bromo-3-cyanobenzoate

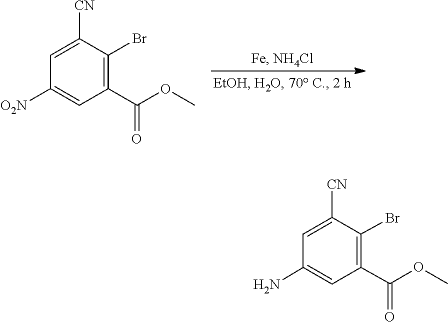

To a solution of methyl 2-bromo-3-cyano-5-nitro-benzoate (30 g, 105 mmol, 1 eq) in EtOH (200 mL) and H₂O (100 mL) was added Fe (13.5 g, 242 mmol, 2.3 eq) and NH₄Cl (12.9 g, 242 mmol, 2.3 eq). The mixture was stirred at 70° C. for 2 h. LCMS showed the reaction was complete and desired mass was detected. The reaction mixture was diluted with H₂O (500 mL) and extracted with ethyl acetate (500 mL×3). The combined organic layers were washed with brine (300 mL×3), dried over Na₂SO₄, filtered, and concentrated under reduced pressure to give a residue. The residue was triturated with MTBE (80 mL) at 25° C. for 1 h to obtain methyl 5-amino-2-bromo-3-cyano-benzoate (22 g, 81.9% yield) as a gray solid. ¹H NMR (400 MHz, DMSO-d₆) δ=7.27-7.00 (m, 2H), 6.05 (s, 2H), 3.84 (s, 3H).

### 12.5 Preparation of 5-amino-2-bromo-3-(hydroxymethyl)benzonitrile

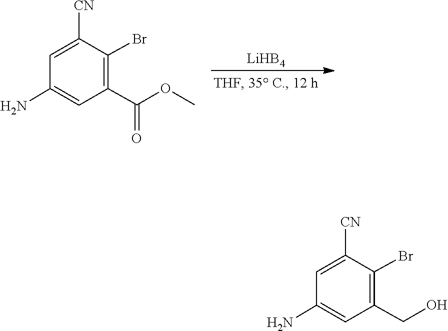

To a solution of methyl 5-amino-2-bromo-3-cyano-benzoate (22 g, 86.2 mmol, 1 eq) in THF (200 mL) was added LiBH₄ (4 M in THF, 64.7 mL, 259 mmol, 3 eq) at 25° C. The mixture was stirred at 35° C. for 12 h. LCMS showed the reaction was complete and desired mass was detected. The reaction mixture was quenched by addition H₂O (300 mL) at 0° C., and adjusted pH to 2 with aq. HCl (2M). The resulting solution was extracted with ethyl acetate (300 mL×3). The combined organic layers were washed with brine (200 mL×2), dried over Na₂SO₄, filtered, and concentrated under reduced pressure to give a residue. The residue was triturated with MTBE (50 mL) at 25° C. for 1 h to obtain 5-amino-2-bromo-3-(hydroxymethyl)benzonitrile (10 g, 51.0% yield) as a yellow solid. ¹H NMR (400 MHz, DMSO-d₆) δ=7.07 (d, J=2.8 Hz, 1H), 6.84 (d, J=2.8 Hz, 1H), 5.79 (s, 2H), 5.52 (t, J=5.6 Hz, 1H), 4.39 (d, J=5.6 Hz, 2H).

### 12.6 Preparation of 2-bromo-5-((4-(((trans)-2-cyanocyclohexyl)amino)-5-methylpyrimidin-2-yl)amino)-3-(hydroxymethyl)benzonitrile

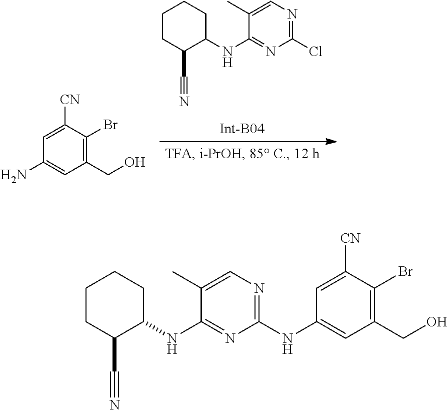

A mixture of 5-amino-2-bromo-3-(hydroxymethyl)benzonitrile (4.12 g, 18.1 mmol, 1.3 eq), 2-((2-chloro-5-methylpyrimidin-4-yl)amino)cyclohexanecarbonitrile (3.5 g, 13.9 mmol, 1 eq), TFA (2.39 g, 20.9 mmol, 1.55 mL, 1.5 eq) in i-PrOH (40 mL) was degassed and purged with N₂ for 3 times, and then the mixture was stirred at 85° C. for 12 h under N₂ atmosphere. LCMS showed the reaction was complete and desired mass was detected. The reaction mixture was filtered and concentrated under reduced pressure to give a residue. The residue was triturated with saturated aq. NaHCO₃ (20 mL) at 25° C. for 2 h to obtain 2-bromo-5-((4-(((trans)-2-cyanocyclohexyl) amino)-5-methylpyrimidin-2-yl)amino)-3-(hydroxymethyl)benzonitrile (2.3 g, 37.3% yield) as a white solid. ¹H NMR (400 MHz, DMSO-d₆) δ=9.50 (s, 1H), 8.37 (d, J=2.4 Hz, 1H), 8.10 (d, J=2.4 Hz, 1H), 7.78 (s, 1H), 6.79 (d, J=8.8 Hz, 1H), 5.63 (s, 1H), 4.50 (s, 2H), 4.41-4.26 (m, 1H), 2.95-2.85 (m, 1H), 2.16 (d, J=11.2 Hz, 1H), 1.99-1.86 (m, 4H), 1.79-1.59 (m, 3H), 1.50-1.27 (m, 2H), 1.25-1.10 (m, 1H).

### 12.7 Preparation of 2-bromo-3-(((tert-butyldimethylsilyl)oxy)methyl)-5-((4-(((trans)-2-cyanocyclohexyl)amino)-5-methylpyrimidin-2-yl)amino)benzonitrile

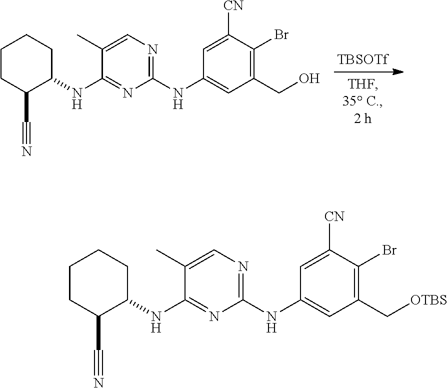

To a solution of 2-bromo-5-((4-(((trans)-2-cyanocyclohexyl)amino)-5-methylpyrimidin-2-yl)amino)-3-(hydroxymethyl)benzonitrile (2.3 g, 5.21 mmol, 1 eq) in THF (50 mL) was added 2,6-dimethylpyridine (1.78 g, 16.6 mmol, 1.94 mL, 3 eq) and [tert-butyl(dimethyl)silyl]trifluoromethanesulfonate (2.20 g, 8.33 mmol, 1.91 mL, 1.5 eq) at 25° C. The mixture was stirred at 35° C. for 2 h. LCMS showed the reaction was consumed completely and one main peak with desired mass was detected. The reaction mixture was diluted with H₂O (100 mL) and extracted with ethyl acetate (100 mL×3). The combined organic layers were washed with brine (100 mL), dried over Na₂SO₄, filtered, and concentrated under reduced pressure to give a residue. The residue was triturated with petroleum ether (50 mL) at 25° C. for 30 min to obtain 2-bromo-3-(((tert-butyldimethylsilyl)oxy)methyl)-5-((4-((2-cyanocyclohexyl)amino)-5-methylpyrimidin-2-yl)amino)benzonitrile (3 g, 97.2% yield) as a white solid. ¹H NMR (400 MHz, DMSO-d₆) δ=9.67 (s, 1H), 8.51 (d, J=2.4 Hz, 1H), 7.97 (d, J=2.0 Hz, 1H), 7.76 (s, 1H), 6.90 (d, J=8.4 Hz, 1H), 4.67 (s, 2H), 4.38-4.22 (m, 1H), 3.00-2.95 (m, 1H), 2.17 (d, J=13.6 Hz, 1H), 1.96 (s, 3H), 1.92-1.84 (m, 1H), 1.80-1.54 (m, 3H), 1.43-1.29 (m, 2H), 1.26-1.08 (m, 1H), 0.94 (s, 9H), 0.13 (s, 6H).

### 12.9 Preparation of (2-(((tert-butyldimethylsilyl)oxy)methyl)-6-cyano-4-((4-(((trans)-2-cyanocyclohexyl)amino)-5-methylpyrimidin-2-yl)amino)phenyl)boronic acid

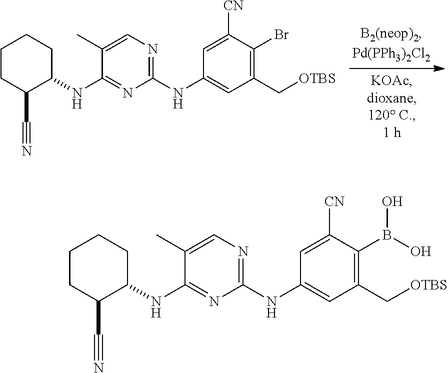

Six reactions were carried out in parallel. For each a mixture of 2-bromo-3-(((tert-butyldimethylsilyl)oxy)methyl)-5-((4-(((trans)-2-cyanocyclohexyl)amino)-5-methylpyrimidin-2-yl)amino)benzonitrile (500 mg, 899 umol, 1 eq), 2-(5,5-dimethyl-1,3,2-dioxaborinan-2-yl)-5,5-dimethyl-1,3,2-dioxaborinane (1.42 g, 6.30 mmol, 7 eq), KOAc (264 mg, 2.70 mmol, 3 eq), Pd(PPh₃)₂Cl₂ (63.1 mg, 90.0 umol, 0.1 eq) in dioxane (10 mL) was degassed and purged with N₂ for 3 times, and then the mixture was stirred at 120° C. for 1 h under N₂ atmosphere. LCMS showed the reaction was consumed completely and desired compound was detected. The six parallel reactions were combined, filtered, and concentrated under reduced pressure to give a residue. The residue was purified by prep-HPLC (neutral condition; column: Waters Xbridge BEH C18 250*50 mm*10 um; mobile phase: [water (NH₄HCO₃)-ACN]; B %: 50%-80%, 10 min) to give (2-(((tert-butyldimethylsilyl)oxy)methyl)-6-cyano-4-((4-(((trans)2-cyanocyclohexyl) amino)-5-methylpyrimidin-2-yl)amino)phenyl)boronic acid (400 mg, 12.5% yield) as a white solid.

### 12.10 Preparation of 5-((4-(((trans)-2-cyanocyclohexyl)amino)-5-methylpyrimidin-2-yl)amino)-1-hydroxy-1,3-dihydrobenzo[c][1,2]oxaborole-7-carbonitrile

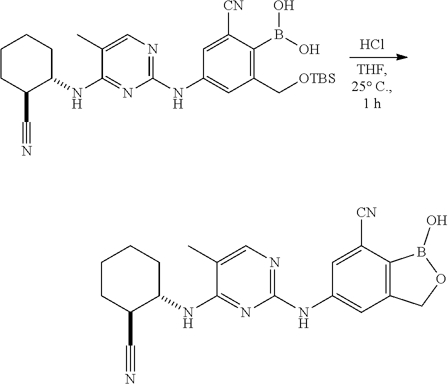

To a solution of (2-(((tert-butyldimethylsilyl)oxy)methyl)-6-cyano-4-((4-((trans)-2-cyanocyclohexyl)amino)-5-methylpyrimidin-2-yl)amino)phenyl)boronic acid (400 mg, 679 umol, 1 eq) in THE (10 mL) was added aq. HCl (6 M, 640 uL, 4 eq) at 25° C. The mixture was stirred at 25° C. for 2 h. Solid was precipitate out after. LCMS showed the reaction was complete and desired MS observed. The reaction mixture was filtered to obtain compound 5-((4-(((trans)-2-cyanocyclohexyl)amino)-5-methylpyrimidin-2-yl)amino)-1-hydroxy-1,3-dihydrobenzo[c][1,2]oxaborole-7-carbonitrile (360 mg, yield 95%, purity 99%) as a white solid.

¹H NMR (400 MHz, DMSO-d₆) δ=11.11 (s, 1H), 9.49-9.35 (m, 1H), 8.65-8.55 (m, 1H), 8.16 (s, 1H), 7.91 (s, 1H), 7.82 (s, 1H), 5.06 (s, 2H), 4.33-4.21 (m, 1H), 3.64-3.56 (m, 1H), 3.12-2.98 (m, 1H), 2.20-2.14 (m, 1H), 2.06 (s, 3H), 1.95-1.87 (m, 1H), 1.74-1.57 (m, 2H), 1.50-1.30 (m, 2H), 1.27-1.13 (m, 1H). The racemic material was separated by SFC (column: DAICEL CHIRALPAK AD (250 mm*30 mm, 10 um); mobile phase: [0.1% NH₃H₂O IPA]; B %: 36%-36%, 6 min) to obtain 5-((4-(((trans)-2-cyanocyclohexyl)amino)-5-methylpyrimidin-2-yl)amino)-1-hydroxy-1,3-dihydrobenzo[c][1,2]oxaborole-7-carbonitrile (80.8 mg, 99.62% purity, 99.42% ee, first peak, Rt=1.108 min) stereoisomer one as a white solid; ¹H NMR (400 MHz, DMSO-d₆) δ=9.55 (s, 1H), 9.17 (s, 1H), 8.23 (s, 1H), 8.02 (s, 1H), 7.80 (s, 1H), 6.82 (d, J=8.8 Hz, 1H), 5.05-4.95 (m, 2H), 4.37-4.25 (m, 1H), 3.00-2.90 (m, 1H), 2.22-2.11 (m, 1H), 2.00-1.95 (s, 4H), 1.81-1.57 (m, 3H), 1.45-1.27 (m, 2H), 1.26-1.12 (m, 1H). MS (ESI): mass calcd. For C₂₀H₂₁BN₆O₂ 388.18, m/z found 389.2 [M+H]⁺. HPLC: 99.62% (220 nm), 99.60% (254 nm). and 5-((4-(((trans)-2-cyanocyclohexyl)amino)-5-methylpyrimidin-2-yl)amino)-1-hydroxy-1,3-dihydrobenzo[c][1,2]oxaborole-7-carbonitrile (72.0 mg, 99.62% purity, 98.64% ee, second peak, Rt=1.499 min) stereoisomer two as a white solid. ¹H NMR (400 MHz, DMSO-d₆) δ=9.58 (s, 1H), 9.19 (s, 1H), 8.23 (s, 1H), 8.02 (s, 1H), 7.80 (s, 1H), 6.84 (d, J=8.8 Hz, 1H), 5.05-4.95 (m, 2H), 4.38-4.25 (m, 1H), 2.98-2.86 (m, 1H), 2.22-2.13 (m, 1H), 2.00-1.95 (s, 4H), 1.82-1.57 (m, 3H), 1.44-1.27 (m, 2H), 1.25-1.12 (m, 1H). MS (ESI): mass calcd. For C₂₀H₂₁BN₆O₂ 388.18, m/z found 387.1 [M−H]⁻. HPLC: 99.62% (220 nm), 100.0% (254 nm).

### 13. Preparation of (trans)-2-((2-((7,8-dihydro-2H-1,6,9-trioxa-9a-borabenzo[c,d]azulen-4-yl)amino)-5-methylpyrimidin-4-yl)amino)cyclohexane-1-carbonitrile

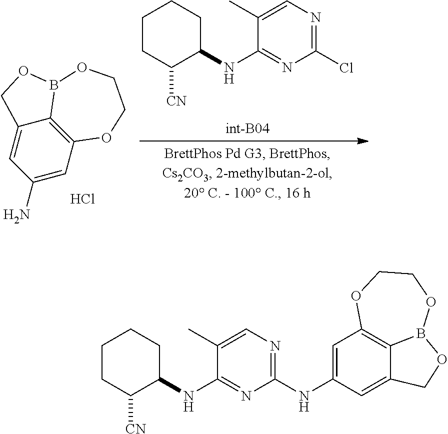

To a mixture of 7,8-dihydro-2H-1,6,9-trioxa-9a-borabenzo[cd]azulen-4-amine (50 mg, 219 umol, 1 eq, HCl) and 2-[(2-chloro-5-methyl-pyrimidin-4-yl)amino]cyclohexanecarbonitrile (60.6 mg, 241 umol, 1.1 eq) in 2-methylbutan-2-ol (3 mL) was added BrettPhos Pd G3 (19.9 mg, 21.9 umol, 0.1 eq), BRETTPHOS (11.8 mg, 21.9 umol, 0.1 eq) and Cs₂CO₃ (214 mg, 659 umol, 3 eq) in one portion at 20° C. under N₂. The mixture was heated to 100° C. and stirred for 16 h. LCMS showed the reaction was complete and desired MS observed. The reaction mixture was filtered and the filtrate was concentrated in vacuum to give a residue. The residue was purified by prep-HPLC (column: Waters Xbridge BEH C18 100*30 mm*10 um; mobile phase: [water (10 mM NH₄HCO₃)-ACN]; B %: 30%-60%, 10 min) to give 2-((2-((7,8-dihydro-2H-1,6,9-trioxa-9a-borabenzo[cd]azulen-4-yl)amino)-5-methylpyrimidin-4-yl)amino)cyclohexane-1-carbonitrile (24 mg, 95.34% purity) as a white solid. ¹H NMR (DMSO-d₆, 400 MHz) δ 9.23 (s, 1H), 7.77 (s, 1H), 7.39 (s, 1H), 7.33 (s, 1H), 6.65 (d, J=6.4 Hz, 1H), 5.06 (s, 2H), 4.66-4.57 (m, 1H), 4.35-4.24 (m, 2H), 4.22-4.12 (m, 1H), 4.03-3.98 (m, 1H), 3.76-3.74 (m, 1H), 2.03-1.99 (m, 1H), 1.98 (s, 3H), 1.88-1.77 (m, 3H), 1.74-1.65 (m, 2H), 1.45-1.39 (m, 2H). MS (ESI): mass calcd. For C₂₁H₂₄BN₅O₃ 405.20, m/z found 404.3 [M−H]⁻. HPLC: 95.34% (220 nm), 90.93% (254 nm).

### 14. Preparation of (trans)-2-[[2-[(1-hydroxy-3,3-dimethyl-2,1-benzoxaborol-5-yl)amino]-5-methyl pyrimidin-4-yl]amino]cyclohexanecarbonitrile hydrochloride

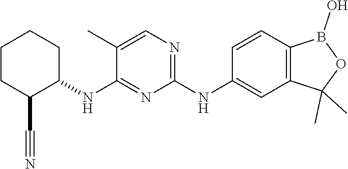

### 14.1 Preparation of methyl 2-bromo-5-(tert-butoxycarbonylamino)benzoate

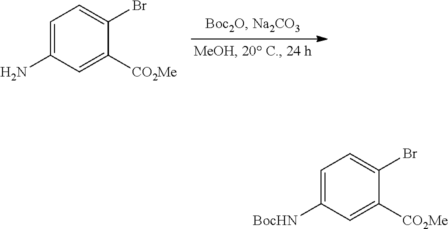 [SMILES: COC(=O)c1cc(N)ccc1Br ; COC(=O)c1cc(NC(=O)OC(C)(C)C)ccc1Br]

To a mixture of methyl 5-amino-2-bromo-benzoate (5.00 g, 21.7 mmol, 1 eq) in MeOH (35 mL) was added Boc₂O (10.4 g, 47.8 mmol, 2.2 eq) and Na₂CO₃ (6.91 g, 65.2 mmol, 3 eq) at 20° C. The mixture was stirred at 20° C. for 24 h. TLC showed the reaction was complete. The reaction was removed methanol under reduced pressure and the resulting residue was quenched with H₂O (50 mL) and extracted with EtOAc (50 mL×3). The combined organic layers were washed with brine (20 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜13% Ethyl acetate/petroleum ether gradient @ 75 mL/min) to give methyl 2-bromo-5-(tert-butoxycarbonylamino)benzoate (6.00 g, 83.6% yield) as a yellow solid. ¹H NMR (CDCl₃, 400 MHz) δ 7.84 (d, J=2.4 Hz, 1H), 7.54 (d, J=8.8 Hz, 1H), 7.40 (dd, J=8.4 Hz, 2.4 Hz, 1H), 6.69 (br s, 1H), 3.91 (s, 3H), 1.51 (s, 9H).

### 14.2 Preparation of 2-(5-amino-2-bromo-phenyl)propan-2-ol

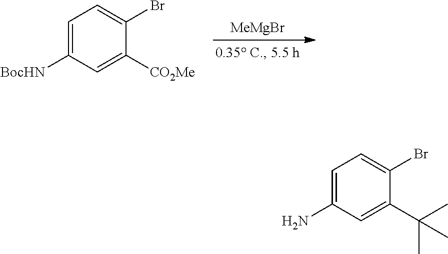 [SMILES: "MeMgBr" ; COC(=O)c1cc(NC(=O)OC(C)(C)C)ccc1Br ; CC(C)(C)c1cc(N)ccc1Br]

To a solution of MeMgBr (3 M in diethyl ether, 48.5 mL, 12 eq) in THE (40 mL) was added methyl 2-bromo-5-(tert-butoxycarbonylamino)benzoate (4.00 g, 12.1 mmol, 1 eq) at 0° C. over 0.5 h. After the addition, the reaction was warmed to 35° C. and stirred for 5.5 h. TLC showed the reaction was complete. The reaction mixture was poured into saturate aq. NH₄Cl (100 mL), adjusted pH to 6 with 2N HCl at 0° C. and extracted with EtOAc (50 mL×3). The combined organic layers were washed with brine (50 mL×3), dried over Na₂SO₄, filtered, and concentrated under reduced pressure to give a residue. The residue was purified by flash silica gel chromatography (ISCO®; 40 g SepaFlash® Silica Flash Column, Eluent of 0˜30% Ethyl acetate/petroleum ether gradient @ 75 mL/min) to give 2-(5-amino-2-bromo-phenyl) propan-2-ol (2.3 g, 82% yield) as a yellow solid. ¹H NMR (CDCl₃, 400 MHz) δ 7.31 (d, J=8.4 Hz, 1H), 7.02 (d, J=2.8 Hz, 1H), 6.44 (dd, J=8.4 Hz, 2.8 Hz, 1H), 1.72 (s, 6H).

### 14.3 Preparation of 2-[[2-[4-bromo-3-(1-hydroxy-1-methyl-ethyl)anilino]-5-methyl-pyrimidin-4-yl]amino]cyclohexanecarbonitrile

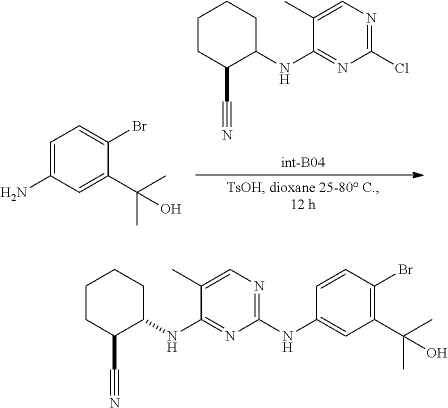

To a solution of 2-[(2-chloro-5-methyl-pyrimidin-4-yl)amino]cyclohexanecarbonitrile (700 mg, 2.79 mmol, 1 eq) in dioxane (20 mL) was added 2-(5-amino-2-bromo-phenyl)propan-2-ol (697 mg, 3.03 mmol, 1.09 eq) and TsOH·H₂O (797 mg, 4.19 mmol, 1.5 eq) at 25° C. under N₂, the mixture was heated and stirred at 80° C. for 12 h. TLC showed the reaction was complete. The reaction mixture was concentrated under reduced pressure to give a residue. The residue was quenched with H₂O (20 mL), adjusted pH to 7 with saturated aq. NaHCO₃ at 0° C. and then extracted with EtOAc (20 mL×3). The combined organic layers were washed with brine (20 mL×3), dried over Na₂SO₄, filtered, and concentrated under reduced pressure to give a residue. The residue was triturated with MTBE (20 mL) at 25° C. to give 2-[[2-[4-bromo-3-(1-hydroxy-1-methyl-ethyl)anilino]-5-methyl-pyrimidin-4-yl] amino]cyclohexanecarbonitrile (700 mg, 56.4% yield) as an off-white solid. ¹H NMR (DMSO-d₆, 400 MHz) δ 8.99 (s, 1H), 8.13 (d, J=2.4 Hz, 1H), 7.71 (s, 1H), 7.64 (dd, J=8.8 Hz, 2.8 Hz, 1H), 7.37 (d, J=8.4 Hz, 1H), 6.63 (d, J=8.8 Hz, 1H), 5.13 (s, 1H), 4.39-4.31 (m, 1H), 2.97-2.90 (m, 1H), 2.16-2.11 (m, 1H), 1.93 (s, 3H), 1.92-1.88 (m, 1H), 1.75-1.66 (m, 3H), 1.61 (s, 6H), 1.45-1.23 (m, 3H).

### 14.4 Preparation of (trans)-2-[[2-[(1-hydroxy-3,3-dimethyl-2,1-benzoxaborol-5-yl)amino]-5-methyl-pyrimidin-4-yl]amino]cyclohexane-1-carbonitrile hydrochloride

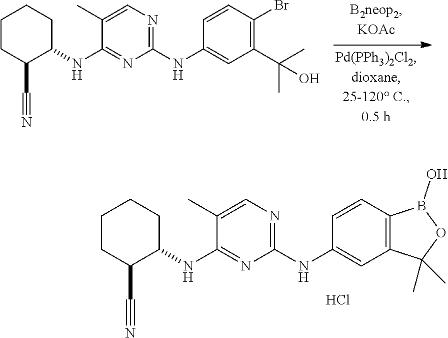

Five reactions were carried out in parallel. For each a solution of (trans)-2-[[2-[4-bromo-3-(1-hydroxy-1-methyl-ethyl)anilino]-5-methyl-pyrimidin-4-yl]amino]cyclohexanecarbonitrile (100 mg, 225 umol, 1 eq) and 2-(5,5-dimethyl-1,3,2-dioxaborinan-2-yl)-5,5-dimethyl-1,3,2-dioxaborinane (127 mg, 563 umol, 2.5 eq) in dioxane (5 ml) was added KOAc (44.2 mg, 450 umol, 2 eq), Pd(PPh₃)₂Cl₂ (15.8 mg, 22.5 umol, 0.1 eq) at 25° C. under N₂. The mixture was heated and stirred at 120° C. for 0.5 h. TLC showed the reaction was complete. The five parallel reactions were combined and filtered. The filtrate was concentrated under reduced pressure to give a residue. The residue was purified by Prep-HPLC (column: Phenomenex luna C18 80*40 mm*3 um; mobile phase: [water (0.04% HCl)-ACN]; B %: 18%-32%, 7 min) to give (trans)-2-[[2-[(1-hydroxy-3,3-dimethyl-2,1-benzoxaborol-5-yl)amino]-5-methyl-pyrimidin-4-yl]amino]cyclohexanecarbonitrile hydrochloride (163.3 mg, 35.2% yield, 95.81% purity) as a white solid. ¹H NMR (DMSO-d₆, 400 MHz) δ 12.17 (s, 1H), 10.42 (s, 1H), 9.03 (s, 1H), 8.36 (s, 1H), 7.84 (s, 1H), 7.68 (d, J=7.6 Hz, 1H), 7.59 (s, 1H), 7.47 (dd, J=9.6 Hz, 8.0 Hz 1H), 4.34-4.25 (m, 1H), 3.08-2.99 (m, 1H), 2.24-2.17 (m, 1H), 2.04 (s, 3H), 1.93-1.85 (m, 1H), 1.78-1.69 (m, 2H), 1.61-1.51 (m, 1H), 1.48 (d, J=10.4 Hz, 6H), 1.44-1.37 (m, 1H), 1.28-1.14 (m, 2H). MS (ESI): mass calcd. For C₂₁H₂₇BClN₅O₂ 391.22, m/z found 392.3 [M+H]⁺. HPLC: 95.81% (220 nm), 96.45% (254 nm).

### 15. Preparation of (trans)-2-[[2-[(2-hydroxy-1,2-benzoxaborinin-6-yl)amino]-5-methyl-pyrimidin-4-yl]amino]cyclohexanecarbonitrile

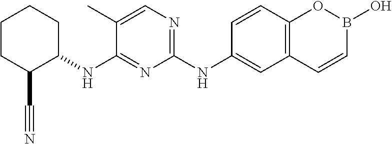

### 15.1 Preparation of 2-[[2-[(4-methoxyphenyl)methylamino]-5-methyl-pyrimidin-4-yl]amino]cyclohexanecarbonitrile

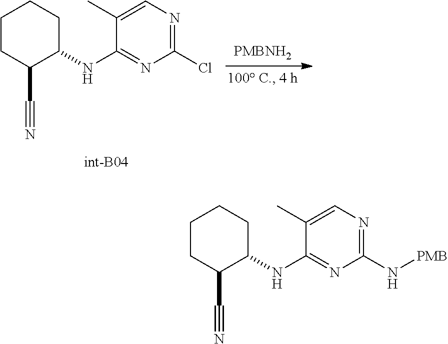

A mixture of (trans)-2-[(2-chloro-5-methyl-pyrimidin-4-yl)amino]cyclohexane-1-carbonitrile (600 mg, 2.39 mmol, 1 eq) and PMBNH₂ (6.36 g, 46 mmol, 6 mL, 19 eq) was stirred at 100° C. for 4 h. TLC showed the reaction was complete. The reaction was poured into saturated aq. NH₄Cl (20 mL) and adjusted pH to 5 with aq. HCl (2N). The aqueous was extracted with EtOAc (20 mL×3). The combined organic layers were washed with brine (30 mL×2), dried over Na₂SO₄, filtered, and concentrated under reduced pressure to give a residue. The residue was purified by column chromatography (SiO₂, Petroleum ether/ethyl acetate=5/1 to 0/1) to give (trans)-2-[[2-[(4-methoxyphenyl)methylamino]-5-methyl-pyrimidin-4-yl]amino]cyclohexane-1-carbonitrile (370 mg, 44% yield) as a white solid. ¹H NMR (CDCl₃, 400 MHz) δ 12.16 (s, 1H), 8.45 (s, 1H), 8.23 (q, J=8.4 Hz, 1H), 7.65 (s, 1H), 8.28 (q, J=8.8 Hz, 2H), 6.89 (q, J=8.4 Hz, 2H), 4.55-4.25 (m, 2H), 4.40-4.20 (m, 1H), 3.72 (s, 3H), 3.10-2.90 (m, 1H), 2.20-2.10 (m, 1H), 1.95 (s, 3H), 1.75-1.65 (m, 2H), 1.63-1.50 (m. 1H), 1.40-1.20 (m, 2H), 1.20-1.00 (m, 1H).

### 15.2 Preparation of 2-[(2-amino-5-methyl pyrimidin-4-yl)amino]cyclohexane-1-carbonitrile

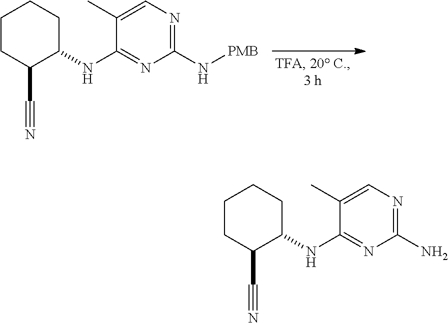

A mixture of (trans)-2-[[2-[(4-methoxyphenyl)methylamino]-5-methyl-pyrimidin-4-yl]amino] cyclohexane-1-carbonitrile (300 mg, 853 umol, 1 eq) in TFA (3 mL) was stirred at 20° C. for 3 h under N₂ atmosphere. TLC showed the reaction was complete. The reaction mixture was concentrated under reduced pressure to remove TFA. The residue was diluted with water 20 mL and extracted with DCM (10 mL×3). The aqueous layer was lyophilized to give (trans)-2-[(2-amino-5-methyl-pyrimidin-4-yl)amino]cyclohexane-1-carbonitrile (200 mg, 96% yield) as a white solid.

### 15.3 Preparation of (trans)-2-[[2-[(2-hydroxy-1,2-benzoxaborinin-6-yl)amino]-5-methyl-pyrimidin-4-yl]amino]cyclohexane-1-carbonitrile

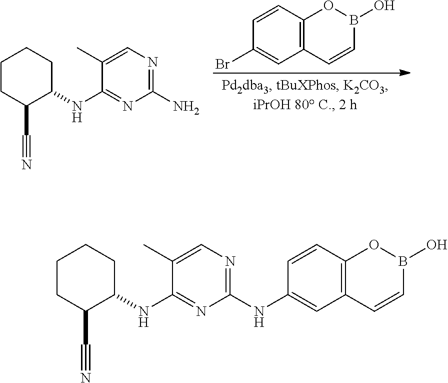

A mixture of 6-bromo-2-hydroxy-1,2-benzoxaborinine (100 mg, 444 umol, 1 eq), (trans)-2-[(2-amino-5-methyl-pyrimidin-4-yl)amino]cyclohexane-1-carbonitrile (103 mg, 444 umol, 1 eq), K₂CO₃ (123 mg, 889 umol, 2 eq), Pd₂(dba)₃ (20 mg, 22 umol, 0.05 eq) and t-Bu Xphos (19 mg, 44 umol, 0.1 eq) in i-PrOH (6 mL) was degassed and purged with N₂ for 3 times, and then the mixture was stirred at 80° C. for 2 h under N₂ atmosphere. TLC showed the reaction was complete. The reaction mixture was quenched by addition saturated aq. NH₄Cl (10 mL) at 20° C., and then extracted with EtOAc (5 mL×3). The combined organic layers were washed with brine (10 mL×3), dried over Na₂SO₄, filtered, and concentrated under reduced pressure to give a residue. The residue was purified by prep-HPLC (column: Waters Xbridge BEH C18 100*30 mm*10 um; mobile phase: [water (10 mM NH₄HCO₃)-ACN]; B %: 25%-50%, 10 min) to give (trans)-2-[[2-[(2-hydroxy-1,2-benzoxaborinin-6-yl)amino]-5-methyl-pyrimidin-4-yl]amino]cyclohexane-1-carbonitrile (31.8 mg, 19% yield) as a white solid. ¹H NMR (DMSO-d₆, 400 MHz) δ 8.85 (s, 1H), 8.80 (s, 1H), 7.89 (s, 1H), 7.80-7.65 (m, 2H), 7.60 (d, J=8.8 Hz, 1H), 7.11 (d, J=8.8 Hz, 1H), 6.63 (d, J=8.8 Hz, 1H), 6.11 (d, J=12.0 Hz, 1H), 4.35-4.25 (m, 1H), 3.00-2.90 (m, 1H), 2.18 (d, J=11.6 Hz, 1H), 2.00-1.90 (m, 4H), 1.85-1.70 (m, 2H), 1.65-1.50 (m, 1H), 1.40-1.25 (m, 2H), 1.25-1.10 (m, 1H). MS (ESI): mass calcd. For C₂₀H₂₂BN₅O₂ 375.19, m/z found 374.3 [M−H]⁻. HPLC: 98.55% (220 nm), 99.53% (254 nm).

### 16. Preparation of (trans)-2-((2-((2-hydroxy-3-methyl-2H-benzo[e][1,2]oxaborinin-6-yl)amino)-5-methylpyrimidin-4-yl)amino)cyclohexane-1-carbonitrile

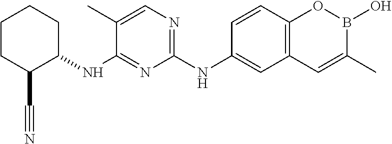

### 16.1 Preparation of tert-butyl N-(2-hydroxy-3-methyl-1,2-benzoxaborinin-6-yl)carbamate

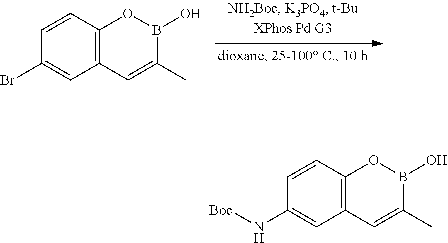 [SMILES: CC1=Cc2cc(Br)ccc2OB1O ; CC1=Cc2cc(NC(=O)OC(C)(C)C)ccc2OB1O]

Three reactions were carried out in parallel. For each a mixture of 6-bromo-2-hydroxy-3-methyl-1,2-benzoxaborinine (200 mg, 837 umol, 1 eq) and tert-butyl carbamate (118 mg, 1.0 mmol, 1.2 eq) in dioxane (3 mL) was added t-Bu Xphos (36 mg, 83.7 umol, 0.1 eq), K₃PO₄ (355 mg, 1.67 mmol, 2 eq) and [2-(2-aminophenyl)phenyl]-methylsulfonyloxy-palladium;ditert-butyl-[2-(2,4,6-triisopropylphenyl)phenyl]phosphane (t-Bu Xphos Pd G3, 66 mg, 83.7 umol, 0.1 eq) in one portion at 25° C. under N₂. The mixture was heated and stirred at 100° C. for 10 h. TLC showed the reaction was complete. The three parallel reactions were combined, filtered and the filtrate was concentrated under reduced pressure to give a residue. The residue was purified by flash silica gel chromatography (ISCO®; 20 g SepaFlash® Silica Flash Column, Eluent of 70˜80% Ethyl acetate/petroleum ether gradient @ 60 mL/min) to give tert-butyl N-(2-hydroxy-3-methyl-1,2-benzoxaborinin-6-yl)carbamate (600 mg, 60% yield, 70% purity) as a white solid. ¹H NMR (DMSO-d₆, 400 MHz) δ 9.26 (s, 1H), 8.81 (s, 1H), 7.53 (s, 1H), 7.39 (s, 1H), 7.31 (d, J=8.8 Hz, 1H), 7.07 (d, J=8.8 Hz, 1H), 2.00 (s, 3H), 1.48 (s, 9H).

### 16.2 Preparation of 2-hydroxy-3-methyl-1,2-benzoxaborinin-6-amine hydrochloride

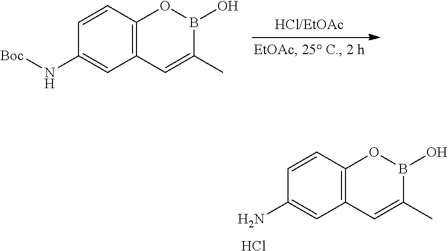 [SMILES: CC1=Cc2cc(NC(=O)OC(C)(C)C)ccc2OB1O ; CC1=Cc2cc(N)ccc2OB1O ; Cl]

To a mixture of tert-butyl N-(2-hydroxy-3-methyl-1,2-benzoxaborinin-6-yl)carbamate (500 mg, 1.27 mmol, 70% purity, 1 eq) in EtOAc (3 mL) was added HCl/EtOAc (4N, 6.36 mL, 20 eq) in one portion at 25° C. under N₂. The mixture was stirred at 25° C. for 2 h. TLC showed the reaction was complete. The mixture was concentrated to give a residue. The residue was triturated with EtOAc (2 mL) to give 2-hydroxy-3-methyl-1,2-benzoxaborinin-6-amine hydrochloride (150 mg, 55% yield, HCl salt) as a yellow solid. ¹H NMR (DMSO-d₆, 400 MHz) δ 10.12 (s, 3H), 9.09 (s, 1H), 7.52 (s, 1H), 7.38 (s, 1H), 7.32-7.27 (m, 2H), 2.03 (s, 3H).

### 16.3 Preparation of (trans)-2-((2-((2-hydroxy-3-methyl-2H-benzo[e][1,2]oxaborinin-6-yl)amino)-5-methylpyrimidin-4-yl)amino)cyclohexane-1-carbonitrile

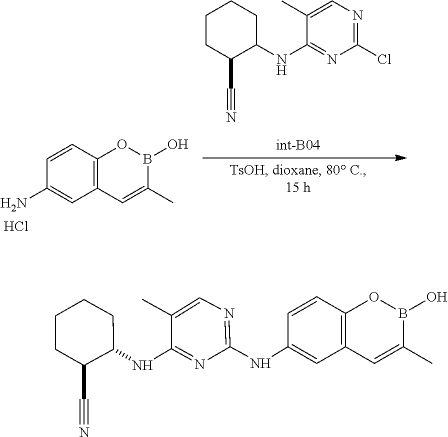

Two reactions were carried out in parallel. For each a mixture of 2-hydroxy-3-methyl-1,2-benzoxaborinin-6-amine hydrochloride (100 mg, 472 umol, 1 eq, HCl salt) and 2-[(2-chloro-5-methyl-pyrimidin-4-yl)amino]cyclohexanecarbonitrile (119 mg, 472 umol, 1 eq) in dioxane (5 mL) was added TsOH·H₂O (81 mg, 472 umol, 1 eq) in one portion at 25° C. under N₂. The mixture was heated and stirred at 80° C. for 15 h. TLC showed the reaction was complete. The two parallel reactions were combined and concentrated under reduced pressure to give a residue. The residue was dissolved in EtOAc (10 mL) and washed with saturated aq. NaHCO₃ (5 mL×3), dried over Na₂SO₄, filtered, and concentrated under reduced pressure to get the residue. The residue was triturated with a mixture solvent of MeCN (5 mL) and H₂O (5 mL) to give 2-((2-((2-hydroxy-3-methyl-2H-benzo[e][1,2]oxaborinin-6-yl)amino)-5-methylpyrimidin-4-yl)amino)cyclohexane-1-carbonitrile (102 mg, 27% yield, 99.68% purity) as a white solid. ¹H NMR (DMSO-d₆, 400 MHz) δ 8.79 (s, 1H), 7.80 (s, 1H), 7.70 (s, 1H), 7.51 (d, J=8.0 Hz, 1H), 7.35 (s, 1H), 7.03 (d, J=8.0 Hz, 1H), 6.62 (d, J=8.8 Hz, 1H), 4.31-4.28 (m, 1H), 2.98-2.91 (m, 1H), 2.19-2.16 (m, 1H), 1.99 (s, 3H), 1.93 (s, 3H), 1.82-1.90 (m, 1H), 1.76-1.69 (m, 2H), 1.59-1.56 (m, 1H), 1.32-1.24 (m, 2H), 1.21-1.14 (m, 1H). MS (ESI): mass calcd. For C₂₁H₂₄BN₅O₂ 389.20, m/z found 390.3 [M+H]⁺. HPLC: 99.68% (220 nm), 99.75% (254 nm).

### 17. Preparation of (trans)-2-((5-chloro-2-((1-hydroxy-1,3-dihydrobenzo[c][1,2]oxaborol-5-yl)amino)pyrimidin-4-yl)amino)cyclohexane-1-carbonitrile

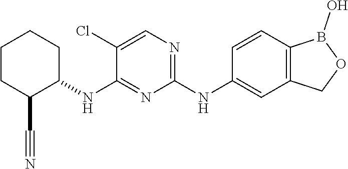

### 17.1 Preparation of methyl 2-bromo-5-((5-chloro-4-((trans)-2-cyanocyclohexyl) amino)pyrimidin-2-yl)amino)benzoate

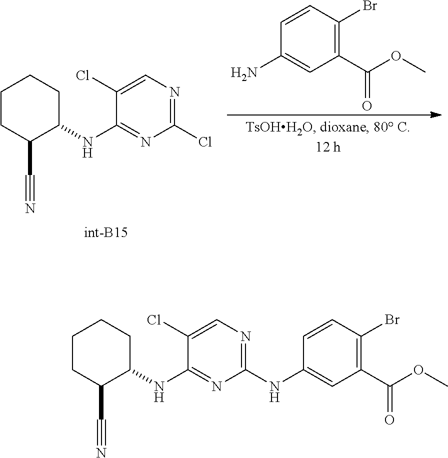

A mixture of (trans)-2-[(2,5-dichloropyrimidin-4-yl)amino]cyclohexane-1-carbonitrile (2.30 g, 8.48 mmol, 1 eq), methyl 5-amino-2-bromo-benzoate (2.15 g, 9.33 mmol, 1.1 eq) and TsOH·H₂O (3.23 g, 16.9 mmol, 2 eq) in dioxane (60 mL) was heated and stirred at 80° C. for 12 h. TLC showed the reaction was complete. The reaction mixture was cooled and concentrated in vacuum to give a residue. The residue was triturated with a mixture solvent of EtOAc (50 mL) and saturated aq. Na₂CO₃ (50 mL) to give methyl 2-bromo-5-[[5-chloro-4-[(2-cyanocyclo hexyl)amino]pyrimidin-2-yl]amino]benzoate (2.1 g, 53% yield) as a gray solid. ¹H NMR (CDCl₃, 400 MHz) δ 8.02 (s, 1H), 7.95 (s, 1H), 7.59-7.54 (m, 2H), 7.15 (s, 1H), 5.27-5.25 (m, 1H), 4.38-4.32 (m, 1H), 3.92 (s, 3H), 2.76-2.73 (m, 1H), 2.12-2.10 (m, 2H), 1.80-1.77 (m, 3H), 1.62-1.58 (m, 1H), 1.47-1.43 (m, 2H).

### 17.2 Preparation of methyl 5-((5-chloro-4-(((trans)-2-cyanocyclohexyl)amino) pyrimidin-2-yl)amino)-2-(5,5-dimethyl-1,3,2-dioxaborinan-2-yl)benzoate

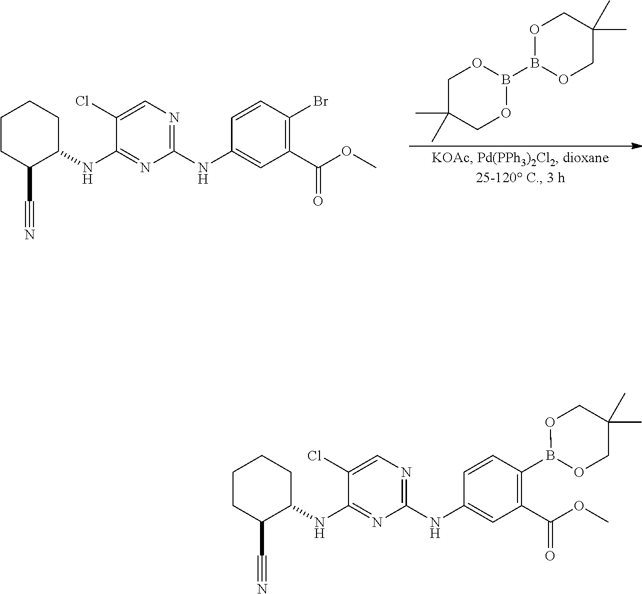

To a mixture of methyl 2-bromo-5-((5-chloro-4-(((trans)-2-cyanocyclohexyl) amino)pyrimidin-2-yl)amino)benzoate (500 mg, 1.08 mmol, 1 eq) in dioxane (15 mL) was added KOAc (264 mg, 2.69 mmol, 2.5 eq), Pd(PPh₃)₂Cl₂ (75.5 mg, 107 umol, 0.1 eq) and 2-(5,5-dimethyl-1,3,2-dioxaborinan-2-yl)-5,5-dimethyl-1,3,2-dioxaborinane (608 mg, 2.69 mmol, 2.5 eq) in one portion at 25° C. under N₂. Then the mixture was heated to 120° C. and stirred for 3 hours. TLC showed the reaction was complete. The reaction mixture was filtered and concentrated under reduced pressure to give a residue. The residue was triturated with MTBE (20 mL) at 20° C. for 30 min to give methyl 5-((5-chloro-4-(((trans)-2-cyanocyclohexyl)amino) pyrimidin-2-yl)amino)-2-(5,5-dimethyl-1,3,2-dioxaborinan-2-yl)benzoate (500 mg, crude) as a off-white solid, which was used directly for next step.

### 17.3 Preparation of (trans)-2-((5-chloro-2-((1-hydroxy-1,3-dihydrobenzo[c][1,2] oxaborol-5-yl)amino)pyrimidin-4-yl)amino)cyclohexane-1-carbonitrile

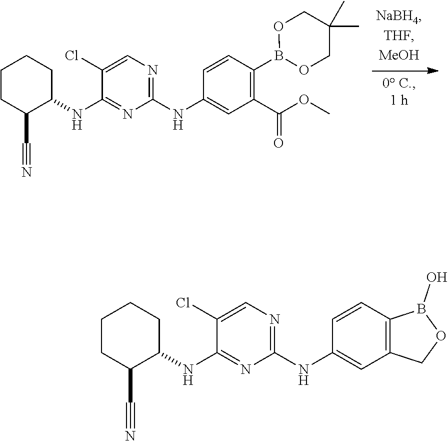

To a mixture of methyl methyl 5-((5-chloro-4-(((trans)-2-cyanocyclohexyl)amino) pyrimidin-2-yl)amino)-2-(5,5-dimethyl-1,3,2-dioxaborinan-2-yl)benzoate (500 mg, 1.00 mmol, 1 eq) in THF (10 mL) was added MeOH (12.3 mmol, 0.5 mL, 12.3 eq) and NaBH₄ (95.0 mg, 2.51 mmol, 2.5 eq) in portions at 0° C. under N₂. The mixture was stirred at 0° C. for 1 h. TLC showed the reaction was complete. The mixture was poured into ice-water (20 mL) and the aqueous phase was adjusted pH to 5-6 with aq. HCl (2N). The resulting mixture was diluted with ethyl acetate (20 mL) and then the suspension was filtered. The filter cake was washed with water (10 mL×3) and dried in vacuum to give a crude product. The crude product was purified by trituration with THF (10 mL) at 20° C. for 0.5 h to give (trans)-2-((5-chloro-2-((1-hydroxy-1,3-dihydrobenzo[c][1,2] oxaborol-5-yl)amino)pyrimidin-4-yl)amino)cyclohexane-1-carbonitrile (132.4 mg, 32% yield, 95.1% purity) as an off-white solid. ¹H NMR (DMSO-d₆, 400 MHz) δ 9.52 (s, 1H), 8.94 (br s, 1H), 8.04 (s, 1H), 7.91 (s, 1H), 7.57 (q, J=8.0 Hz, 2H), 7.35 (d, J=8.8 Hz, 1H), 4.99-4.90 (m, 2H), 4.33-4.27 (m, 1H), 3.05-3.04 (m, 1H), 2.20-2.17 (m, 1H), 1.95-1.91 (m, 1H), 1.80-1.79 (m, 1H), 1.76-1.71 (m, 1H), 1.63-1.59 (m, 1H), 1.37-1.35 (m, 1H), 1.34-1.20 (m, 2H). MS (ESI): mass calcd. For C₁₈H₁₉BClN₅O₂ 383.64, m/z found 384.0 [M+H]⁺. HPLC: 95.1% (220 nm), 93.88% (254 nm).

### 18. Preparation of 5-((4-(((trans)-2-cyanocyclopentyl)amino)-5-methylpyrimidin-2-yl)amino)-1-hydroxy-1,3-dihydrobenzo[c][1,2]oxaborole-7-carbonitrile

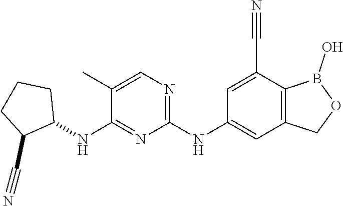 [SMILES: Cc1cnc(Nc2cc(C#N)c3c(c2)COB3O)nc1N[C@H]1CCC[C@@H]1C#N]

### 18.1 Preparation of 2-bromo-5-((4-(((trans)-2-cyanocyclopentyl)amino)-5-methylpyrimidin-2-yl)amino)-3-(hydroxymethyl)benzonitrile

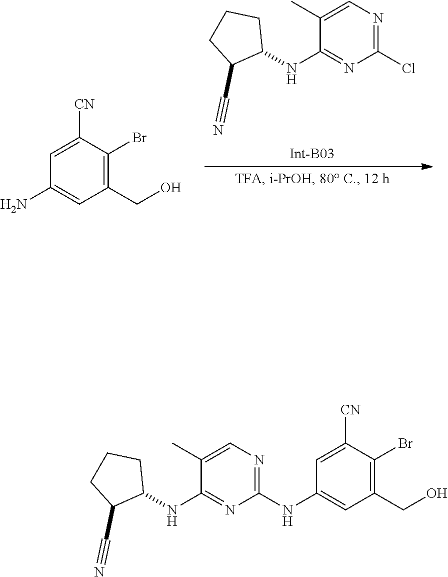

To a solution of 5-amino-2-bromo-3-(hydroxymethyl)benzonitrile (3 g, 13.2 mmol, 1 eq) in i-PrOH (25 mL) was added TFA (2.26 g, 19.8 mmol, 1.47 mL, 1.5 eq) and 2-((2-chloro-5-methylpyrimidin-4-yl)amino)cyclopentanecarbonitrile (3.13 g, 13.3 mmol, 1 eq) at 25° C. The mixture was stirred at 80° C. for 12 h. Solid was precipitate out after 3 h reaction. TLC showed the reaction was complete. The reaction mixture was filtered to give a filter cake. The filter cake was triturated with saturated aq. NaHCO₃ (20 mL) at 25° C. for 30 min to give 2-bromo-5-((4-(((trans)-2-cyanocyclopentyl)amino)-5-methylpyrimidin-2-yl)amino)-3-(hydroxymethyl) benzonitrile (3.6 g, 63.7% yield) as a white solid. ¹H NMR (400 MHz, DMSO-d₆) δ=9.51 (s, 1H), 8.36 (d, J=2.4 Hz, 1H), 8.23 (d, J=2.4 Hz, 1H), 7.80 (s, 1H), 6.88 (d, J=8.4 Hz, 1H), 5.63 (s, 1H), 4.90-4.80 (m, 1H), 4.51 (s, 2H), 3.13 (q, J=8.8 Hz, 1H), 2.26-2.10 (m, 2H), 1.98 (s, 3H), 1.94-1.74 (m, 3H), 1.63-1.50 (m, 1H).

### 18.2 Preparation of 2-bromo-3-(((tert-butyldimethylsilyl)oxy)methyl)-5-((4-(((trans)-2-cyanocyclopentyl)amino)-5-methylpyrimidin-2-yl)amino)benzonitrile

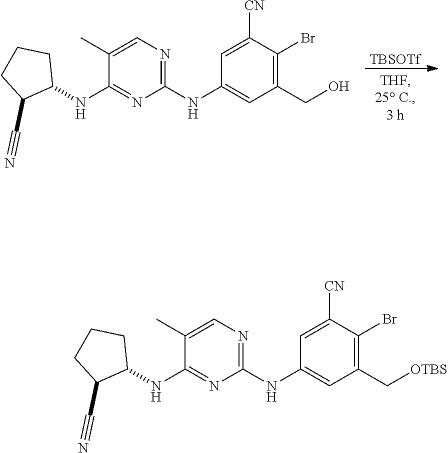

To a solution of 2-bromo-5-((4-(((trans)-2-cyanocyclopentyl)amino)-5-methylpyrimidin-2-yl)amino)-3-(hydroxymethyl)benzonitrile (3.6 g, 8.43 mmol, 1 eq) in THF (35 mL) was added 2,6-dimethylpyridine (2.71 g, 25.2 mmol, 2.94 mL, 3 eq) and [tert-butyl(dimethyl)silyl]trifluoromethanesulfonate (3.34 g, 12.6 mmol, 2.90 mL, 1.5 eq) at 25° C. The mixture was stirred at 25° C. for 2 h. TLC showed the reaction was complete. The reaction mixture was quenched by addition of saturated aq. NH₄Cl (80 mL) at 0° C., and then extracted with EtOAc (80 mL×3). The combined organic layers were washed with brine (50 mL×2), dried over Na₂SO₄, filtered, and concentrated under reduced pressure to give a residue. The residue was triturated with Petroleum ether (10 mL) at 25° C. for 30 min to give compound 2-bromo-3-(((tert-butyldimethylsilyl)oxy)methyl)-5-((4-(((trans)-2-cyanocyclopentyl)amino)-5-methylpyrimidin-2-yl)amino)benzonitrile (3 g, 65.7% yield) as a white solid. ¹H NMR (400 MHz, DMSO-d₆) δ=9.60 (s, 1H), 8.51 (d, J=2.8 Hz, 1H), 8.02 (d, J=2.0 Hz, 1H), 7.77 (s, 1H), 6.86 (d, J=8.0 Hz, 1H), 4.85-4.75 (m, 1H), 4.67 (s, 2H), 3.11 (q, J=8.4 Hz, 1H), 2.26-2.06 (m, 2H), 1.96 (s, 3H), 1.89-1.73 (m, 3H), 1.63-1.52 (m, 1H), 0.94 (s, 9H), 0.13 (s, 6H).

### 18.3 Preparation of 3-(((tert-butyldimethylsilyl)oxy)methyl)-5-((4-(((trans)-2-cyanocyclopentyl)amino)-5-methylpyrimidin-2-yl)amino)-2-(5,5-dimethyl-1,3,2-dioxaborinan-2-yl)benzonitrile

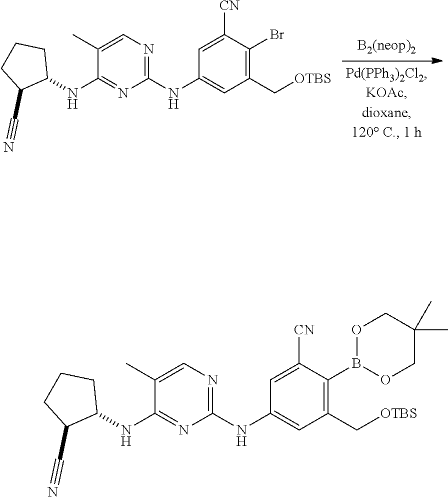

Six reactions were carried out in parallel. For each a mixture of 2-bromo-3-(((tert-butyldimethylsilyl)oxy)methyl)-5-((4-(((trans)-2-cyanocyclopentyl)amino)-5-methylpyrimidin-2-yl)amino)benzonitrile (500 mg, 923 umol, 1 eq), 2-(5,5-dimethyl-1,3,2-dioxaborinan-2-yl)-5,5-dimethyl-1,3,2-dioxaborinane (834 mg, 3.69 mmol, 4 eq), KOAc (271 mg, 2.77 mmol, 3 eq), Pd(PPh₃)₂Cl₂ (64.8 mg, 92.3 umol, 0.1 eq) in dioxane (10 mL) was degassed and purged with N₂ for 3 times, and then the mixture was stirred at 120° C. for 1 h under N₂ atmosphere. LCMS showed the reaction was complete and desired MS observed. The six parallel reactions were combined, diluted with H₂O (150 mL) and extracted with ethyl acetate (30 mL×3). The combined organic layers were washed with brine (70 mL), dried over Na₂SO₄, filtered, and concentrated under reduced pressure to give a residue. The residue was purified by prep-HPLC (neutral condition; column: Waters Xbridge BEH C18 250*50 mm*10 um; mobile phase: [water (NH₄HCO₃)-ACN]; B %: 40%-70%, 10 min) to give 3-(((tert-butyldimethylsilyl)oxy)methyl)-5-((4-(((trans)-2-cyanocyclopentyl)amino)-5-methylpyrimidin-2-yl)amino)-2-(5,5-dimethyl-1,3,2-dioxaborinan-2-yl)benzonitrile (900 mg, 28.2% yield) as a white solid.

### 18.4 Preparation of 5-((4-(((trans)-2-cyanocyclopentyl)amino)-5-methylpyrimidin-2-yl)amino)-1-hydroxy-1,3-dihydrobenzo[c][1,2]oxaborole-7-carbonitrile

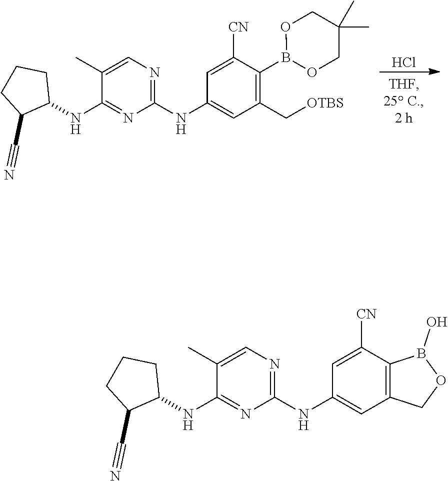

To a solution of 3-(((tert-butyldimethylsilyl)oxy)methyl)-5-((4-(((trans)-2-cyanocyclopentyl) amino)-5-methylpyrimidin-2-yl)amino)-2-(5,5-dimethyl-1,3,2-dioxaborinan-2-yl)benzonitrile (900 mg, 1.57 mmol, 1 eq) in THF (5 mL) was added HCl (6 M, 2 mL, 7.66 eq). The mixture was stirred at 25° C. for 2 h. LCMS showed the reaction was complete and one main peak with desired mass was detected. The reaction was concentrated under reduced pressure to give a residue. The residue was purified by prep-HPLC (formic acid (FA) condition; column: Phenomenex luna C18 (250*70 mm, 15 um); mobile phase: [water (FA)-ACN]; B %: 15%-45%, 20 min) to give 5-((4-(((trans)-2-cyanocyclopentyl)amino)-5-methylpyrimidin-2-yl)amino)-1-hydroxy-1,3-dihydrobenzo[c][1,2]oxaborole-7-carbonitrile (160.5 mg, 28.5% yield) as a white solid. ¹H NMR (400 MHz, DMSO-d₆) δ=9.55 (s, 1H), 9.17 (s, 1H), 8.22 (d, J=1.6 Hz, 1H), 8.09 (s, 1H), 7.80 (s, 1H), 6.88 (d, J=8.0 Hz, 1H), 5.01 (s, 2H), 4.84-4.75 (m, 1H), 3.32-3.07 (m, 1H), 2.25-2.08 (m, 2H), 1.97 (s, 3H), 1.92-1.72 (m, 3H), 1.66-1.51 (m, 1H). MS (ESI): mass calcd. For C₁₉H₁₉BN₆O₂ 374.17, m/z found 375.1 [M+H]⁺. HPLC: 96.77% (220 nm), 95.94% (254 nm).

### 19. Preparation of (trans)-2-((5-chloro-2-((7-chloro-1-hydroxy-1,3-dihydrobenzo[c][1,2]oxaborol-5-yl)amino)pyrimidin-4-yl)amino)cyclohexane-1-carbonitrile

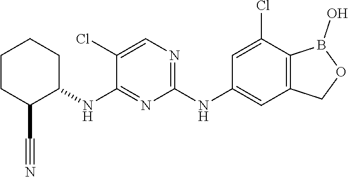

### 19.1 Preparation of 2-((2-((4-bromo-3-chloro-5-(hydroxymethyl)phenyl)amino)-5-chloropyrimidin-4-yl)amino)cyclohexanecarbonitrile

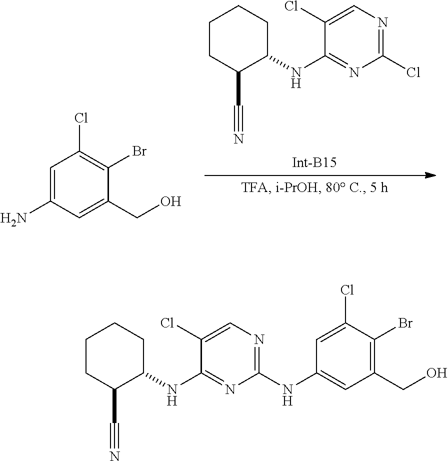

To a solution of (5-amino-2-bromo-3-chloro-phenyl)methanol (800 mg, 3.38 mmol, 1 eq) in i-PrOH (10 mL) was added TFA (578 mg, 5.07 mmol, 375 uL, 1.5 eq) and (trans)-2-[(2,5-dichloropyrimidin-4-yl)amino]cyclohexanecarbonitrile (917 mg, 3.38 mmol, 1 eq) at 25° C. The mixture was stirred at 80° C. for 5 h. Solid was precipitate out. TLC showed the reaction was complete. The reaction mixture was filtered to give a filter cake. The filter cake was triturated with saturated aq. NaHCO₃ (10 mL) to give (trans)-2-((2-((4-bromo-3-chloro-5-(hydroxymethyl)phenyl)amino)-5-chloropyrimidin-4-yl)amino) cyclohexanecarbonitrile (1 g, 62.7% yield) as a yellow solid. ¹H NMR (400 MHz, DMSO-d₆) δ=9.65 (s, 1H), 8.09-8.03 (m, 1H), 7.83 (s, 1H), 7.36 (d, J=9.2 Hz, 1H), 5.56 (s, 1H), 4.49 (s, 2H), 4.41-4.29 (m, 1H), 3.60 (t, J=6.4 Hz, 1H), 3.08-2.96 (m, 1H), 2.20-2.10 (m, 1H), 1.95-1.85 (s, 1H), 1.75-1.58 (m, 3H), 1.46-1.31 (m, 2H), 1.24-1.09 (m, 1H).

### 19.2 Preparation of (trans)-2-((2-((4-bromo-3-(((tert-butyldimethylsilyl)oxy)methyl)-5-chlorophenyl)amino)-5-chloropyrimidin-4-yl)amino)cyclohexanecarbonitrile

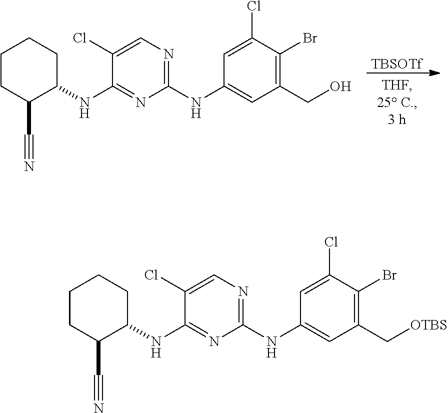

To a solution of (trans)-2-((2-((4-bromo-3-chloro-5-(hydroxymethyl)phenyl)amino)-5-chloropyrimidin-4-yl)amino) cyclohexanecarbonitrile (900 mg, 1.91 mmol, 1 eq) in THE (20 mL) was added 2,6-dimethylpyridine (818 mg, 7.64 mmol, 4 eq) and [tert-butyl(dimethyl)silyl]trifluoromethanesulfonate (757 mg, 2.87 mmol, 1.5 eq) at 25° C. The mixture was stirred at 25° C. for 3 h. TLC showed the reaction was complete. The reaction mixture was quenched by addition of saturated aq. NH₄Cl (30 mL) at 0° C., and then extracted with EtOAc (30 mL×3). The combined organic layers were washed with brine (30 mL×2), dried over Na₂SO₄, filtered, and concentrated under reduced pressure to give a residue. The residue was triturated with petroleum ether (10 mL) to give (trans)-2-((2-((4-bromo-3-(((tert-butyldimethylsilyl)oxy) methyl)-5-chlorophenyl)amino)-5-chloropyrimidin-4-yl)amino)cyclohexanecarbonitrile (600 mg, 53.6% yield) as a white solid. ¹H NMR (400 MHz, DMSO-d₆) δ=9.78 (s, 1H), 8.27 (d, J=2.4 Hz, 1H), 8.03 (s, 1H), 7.62 (s, 1H), 7.39 (d, J=8.8 Hz, 1H), 4.66 (s, 2H), 4.36-4.25 (m, 1H), 3.02 (t, J=2.8, 12.4 Hz, 1H), 2.22-2.13 (m, 1H), 1.95-1.85 (m, 1H), 1.79-1.67 (m, 2H), 1.64-1.50 (m, 1H), 1.48-1.28 (m, 2H), 1.24-1.10 (m, 1H), 0.94 (s, 9H), 0.13 (s, 6H).

### 19.3 Preparation of (trans)-2-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino) cyclohexane-1-carbonitrile

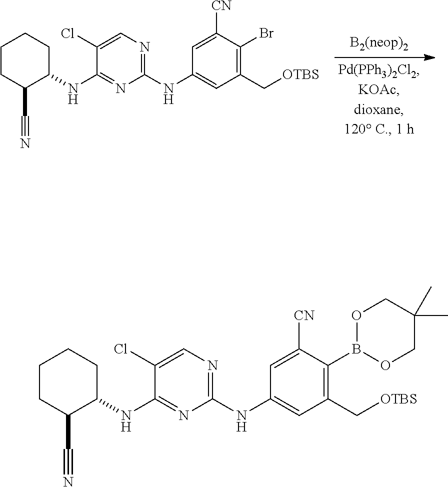

Two reactions were carried out in parallel. For each a mixture of (trans)-2-((2-((4-bromo-3-(((tert-butyldimethylsilyl)oxy)methyl)-5-chlorophenyl)amino)-5-chloropyrimidin-4-yl)amino)cyclohexanecarbonitrile (200 mg, 341 umol, 1 eq), 2-(5,5-dimethyl-1,3,2-dioxaborinan-2-yl)-5,5-dimethyl-1,3,2-dioxaborinane (540 mg, 2.39 mmol, 7 eq), KOAc (100 mg, 1.02 mmol, 3 eq), Pd(PPh₃)₂Cl₂ (24 mg, 34.1 umol, 0.1 eq) in dioxane (3 mL) was degassed and purged with N₂ for 3 times, and then the mixture was stirred at 120° C. for 1 h under N₂ atmosphere. LCMS showed the reaction was complete and desired MS observed. The two parallel reactions were combined and filtered, then the residue was purified by prep-HPLC (neutral condition; column: Waters Xbridge Prep OBD C18 150*40 mm*10 um; mobile phase: [water (NH₄HCO₃)-ACN]; B %: 50%-90%, 8 min) to give (trans)-2-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino)cyclohexane-1-carbonitrile (60 mg, 15% yield) as a white solid.

### 19.4 Preparation of (trans)-2-((5-chloro-2-((7-chloro-1-hydroxy-1,3-dihydrobenzo [c][1,2]oxaborol-5-yl)amino)pyrimidin-4-yl)amino)cyclohexane-1-carbonitrile

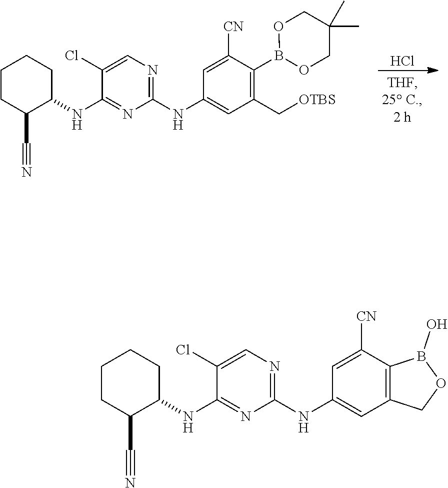

To a solution of (trans)-2-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino)cyclohexane-1-carbonitrile (60 mg, 109 umol, 1 eq) in THE (5 mL) was added aq. HCl (6 M, 0.2 mL) at 25° C. The mixture was stirred at 25° C. for 1 h. LCMS showed the reaction was complete and desired MS observed. The reaction was concentrated under reduced pressure to give a residue. The residue was triturated with n-pentane (1 mL) to give (trans)-2-((5-chloro-2-((7-chloro-1-hydroxy-1,3-dihydrobenzo[c][1,2]oxaborol-5-yl)amino)pyrimidin-4-yl)amino)cyclohexane-1-carbonitrile (18.9 mg, 41.4% yield) as a white solid. ¹H NMR (400 MHz, DMSO-d₆) δ=9.72 (s, 1H), 8.91 (s, 1H), 8.07 (s, 1H), 7.82 (s, 1H), 7.66 (s, 1H), 7.43 (d, J=8.8 Hz, 1H), 5.05-4.95 (m, 2H), 4.36-4.24 (m, 1H), 3.03 (t, J=2.8, 12.0 Hz, 1H), 2.20-2.15 (d, J=12.4 Hz, 1H), 1.95-1.87 (m, 1H), 1.81-1.68 (m, 2H), 1.67-1.55 (m, 1H), 1.44-1.29 (m, 2H), 1.24-1.13 (m, 1H) MS (ESI): mass calcd. For C₁₈H₁₈BCl₂N₅O₂ 417.09, m/z found 418.0 [M+H]⁺. HPLC: 97.31% (220 nm), 98.50% (254 nm).

### 20. Preparation of (trans)-2-[[2-[(7-chloro-1-hydroxy-3H-2,1-benzoxaborol-5-yl)amino]-5 fluoro pyrimidin-4-yl]amino]cyclohexanecarbonitrile

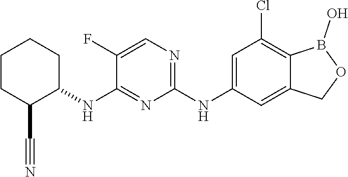

### 20.1 Preparation of (trans)-2-[[2-[4-bromo-3-chloro-5-(hydroxymethyl)anilino]-5 fluoro-pyrimidin-4-yl]amino]cyclohexanecarbonitrile

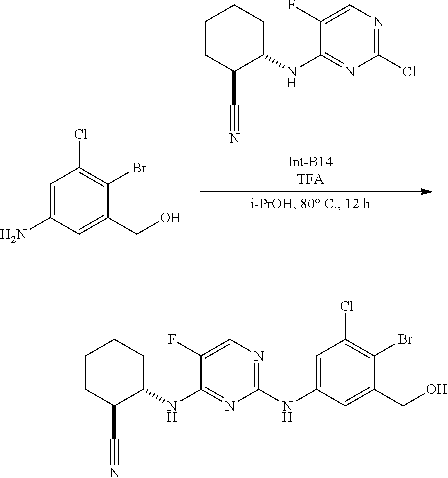

To a solution of (5-amino-2-bromo-3-chloro-phenyl)methanol (1 g, 4.22 mmol, 1 eq) and (trans)-2-[(2-chloro-5-fluoro-pyrimidin-4-yl)amino]cyclohexanecarbonitrile (1.07 g, 4.22 mmol, 1 eq) in i-PrOH (10 mL) was dropwise added TFA (1.44 g, 12.7 mmol, 3 eq) at 25° C. The resulting mixture was stirred at 80° C. for 12 h. Solid was precipitate out after 3 hour reaction. TLC showed the reaction was complete. The reaction mixture was filtered to give a residue. The residue was triturated with saturated aq. NaHCO₃ (20 mL) and filtered to give (trans)-2-[[2-[4-bromo-3-chloro-5-(hydroxymethyl)anilino]-5-fluoro-pyrimidin-4-yl]amino]cyclohexanecarbonitrile (1 g, 52% yield) as a white solid. ¹H NMR (DMSO-d₆, 400 MHz) δ 10.37 (s, 1H), 8.76 (s, 1H), 8.20 (d, J=4.4 Hz, 1H), 7.97 (d, J=2.8 Hz, 1H), 7.79 (d, J=2.8 Hz, 1H), 4.52 (s, 2H), 4.32-4.20 (m, 1H), 2.93-2.75 (m, 1H), 2.20-2.10 (m, 1H), 2.02-1.93 (m, 1H), 1.78-1.61 (m, 3H), 1.41-1.35 (m, 2H), 1.34-1.23 (m, 1H).

### 20.2 Preparation of (trans)-2-((2-((4-bromo-3-(((tert-butyldimethylsilyl)oxy)methyl)-5-chlorophenyl)amino)-5 fluoropyrimidin-4-yl)amino)cyclohexane-1-carbonitrile

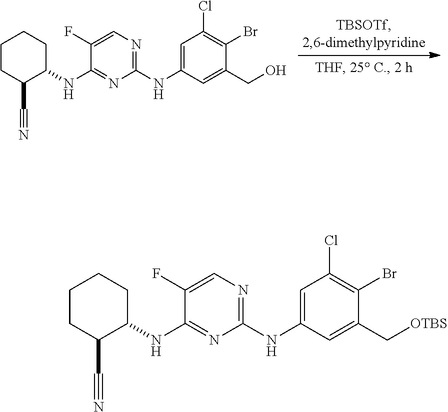

To a solution of (trans)-2-[[2-[4-bromo-3-chloro-5-(hydroxymethyl)anilino]-5-fluoro-pyrimidin-4-yl]amino]cyclohexanecarbonitrile (803 mg, 1.77 mmol, 1 eq) in THF (10 mL) was added 2,6-dimethylpyridine (757 mg, 7.07 mmol, 820 uL, 4 eq) and [tert-butyl(dimethyl)silyl]trifluoromethanesulfonate (700 mg, 2.65 mmol, 600 uL, 1.5 eq) at 25° C. The mixture was stirred at 25° C. for 2 h. TLC showed the reaction was complete. The reaction mixture was quenched by addition of H₂O (20 mL) at 0° C. and extracted with EtOAc (20 mL×3). The combined organic layers were washed with brine (20 mL×2), dried over Na₂SO₄, filtered, and concentrated under reduced pressure to give a residue. The residue was purified by column chromatography (SiO₂, Petroleum ether/ethyl acetate=10/1 to 5/1) to give (trans)-2-((2-((4-bromo-3-(((tert-butyldimethylsilyl)oxy)methyl)-5-chlorophenyl)amino)-5-fluoropyrimidin-4-yl)amino)cyclohexane-1-carbonitrile (0.8 g, 79.6% yield) as a white solid.

### 20.3 Preparation of (trans)-2-((2-((3-(((tert-butyldimethylsilyl)oxy)methyl)-5-chloro-4-(5,5-dimethyl-1,3,2-dioxaborinan-2-yl)phenyl)amino)-5 fluoropyrimidin-4-yl)amino)cyclohexane-1-carbonitrile

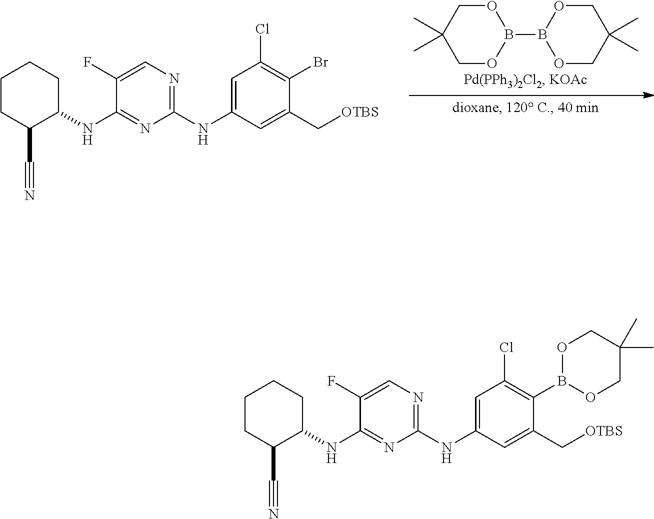

Five reactions were carried out in parallel. For each a mixture of (trans)-2-[[2-[4-bromo-3-[[tert-butyl(dimethyl) silyl]oxymethyl]-5-chloro-anilino]-5-fluoro-pyrimidin-4-yl]amino]cyclohexanecarbonitrile (100 mg, 175 umol, 1 eq), 2-(5,5-dimethyl-1,3,2-dioxaborinan-2-yl)-5,5-dimethyl-1,3,2-dioxaborinane (198 mg, 878 umol, 5 eq), KOAc (52 mg, 527 umol, 3 eq) and Pd(PPh₃)₂Cl₂ (12 mg, 17.5 umol, 0.1 eq) in dioxane (5 mL) was degassed and purged with N₂ for 3 times, and then the mixture was stirred at 120° C. for 40 min under N₂ atmosphere. TLC showed the reaction was complete. The five parallel reactions were combined, filtered and the filtrate was concentrated under reduced pressure to give a residue. The residue was purified by column chromatography (SiO₂, Petroleum ether/ethyl acetate=3/1 to 1/1) to give (trans)-2-((2-((3-(((tert-butyldimethylsilyl)oxy)methyl)-5-chloro-4-(5,5-dimethyl-1,3,2-dioxaborinan-2-yl)phenyl)amino)-5-fluoropyrimidin-4-yl)amino)cyclohexane-1-carbonitrile (0.5 g, crude) as a white solid.

### 20.4 Preparation of (trans)-2-[[2-[(7-chloro-1-hydroxy-3H-2,1-benzoxaborol-5-yl)amino]-5 fluoro-pyrimidin-4-yl]amino]cyclohexanecarbonitrile

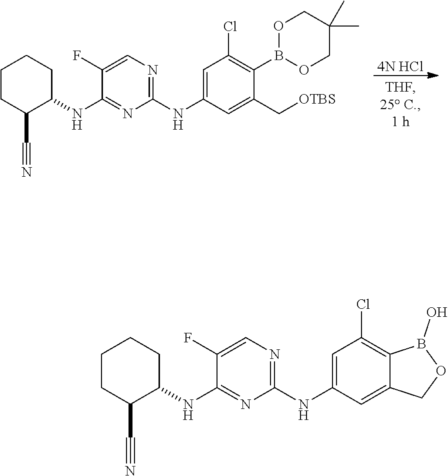

To a solution of (trans)-2-((2-((3-(((tert-butyldimethylsilyl)oxy)methyl)-5-chloro-4-(5,5-dimethyl-1,3,2-dioxaborinan-2-yl)phenyl)amino)-5-fluoropyrimidin-4-yl)amino)cyclohexane-1-carbonitrile (0.5 g, 830 umol, 1 eq) in THE (10 mL) was added aq. HCl (4 M, 2.0 mL, 9.6 eq) at 25° C. The mixture was stirred at 25° C. for 1 h. Solid was precipitate out. The reaction mixture was filtered to give a residue. The residue was triturated with MeCN (5 mL) and filtered to give 2-[[2-[(7-chloro-1-hydroxy-3H-2,1-benzoxaborol-5-yl)amino]-5-fluoro-pyrimidin-4-yl]amino]cyclohexanecarbonitrile (81 mg, 24.3% yield, 98.07% purity) as a white solid. ¹H NMR (DMSO-d₆, 400 MHz) δ 9.59 (s, 1H), 8.89 (s, 1H), 8.01 (d, J=3.6 Hz, 1H), 7.85-7.75 (m, 2H), 7.69 (s, 1H), 5.00-4.90 (m, 2H), 4.32-4.20 (m, 1H), 2.92-2.63 (m, 1H), 2.38-2.30 (m, 1H), 1.86-1.77 (m, 1H), 1.76-1.75 (m, 2H), 1.74-1.70 (m, 1H), 1.41-1.35 (m, 2H), 1.34-1.30 (m, 1H). MS (ESI): mass calcd. For C₁₈H₁₈BClFN₅O₂ 401.12, m/z found 402.2 [M+H]⁺. HPLC: 98.07% (220 nm), 98.08% (254 nm).

### 21. Preparation of (trans)-2-((2-((7-chloro-1-hydroxy-1,3-dihydrobenzo[c][1,2]oxaborol-5-yl)amino)-5-(trifluoromethyl)pyrimidin-4-yl)amino)cyclohexanecarbonitrile

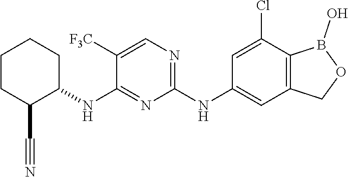

### 21.1 Preparation of (trans)-2-((2-((4-bromo-3-chloro-5-(hydroxymethyl)phenyl) amino)-5-(trifluoromethyl)pyrimidin-4-yl)amino)cyclohexanecarbonitrile

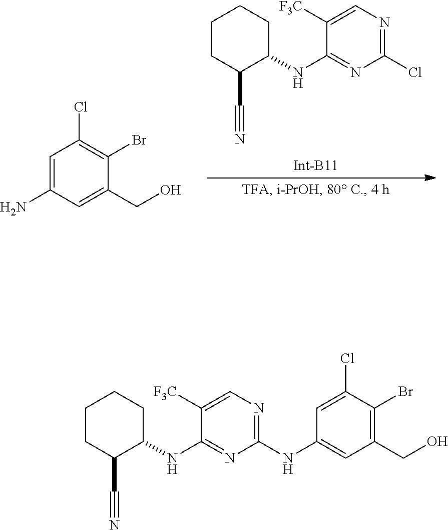

To a solution of (5-amino-2-bromo-3-chloro-phenyl)methanol (500 mg, 2.11 mmol, 1 eq) in i-PrOH (5 mL) was added TFA (361 mg, 3.17 mmol, 1.5 eq) and (trans)-2-[[2-chloro-5-(trifluoromethyl)pyrimidin-4-yl]amino]cyclohexanecarbonitrile (643 mg, 2.11 mmol, 1 eq) at 25° C. The mixture was stirred at 80° C. for 4 hrs. Solid was precipitate out. TLC showed the reaction was complete. The reaction mixture was filtered and the filter cake was triturated with saturated aq. NaHCO₃ (30 mL) and filtered to give (trans)-2-((2-((4-bromo-3-chloro-5-(hydroxymethyl)phenyl)amino)-5-(trifluoromethyl)pyrimidin-4-yl)amino) cyclohexane carbonitrile (800 mg, 75.1% yield) as a brown solid. ¹H NMR (400 MHz, DMSO-d₆) δ=10.36 (s, 1H), 8.36 (s, 1H), 8.05 (s, 1H), 7.79 (s, 1H), 7.51 (d, J=6.0 Hz, 1H), 4.51 (s, 2H), 4.50-4.40 (m, 1H), 3.20-3.10 (m, 1H), 2.20-2.09 (m, 1H), 1.90-1.79 (m, 1H), 1.78-1.54 (m, 3H), 1.52-1.28 (m, 2H), 1.21-1.05 (m, 1H).

### 21.2 Preparation of (trans)-2-((2-((4-bromo-3-(((tert-butyldimethylsilyl)oxy)methyl)-5-chlorophenyl)amino)-5-(trifluoromethyl)pyrimidin-4-yl)amino)cyclohexanecarbonitrile

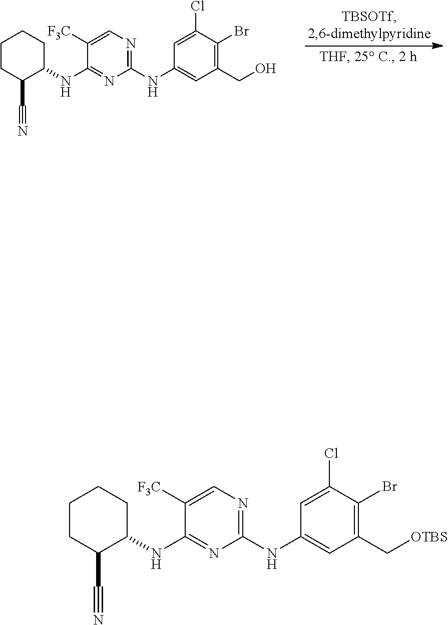

To a solution of (trans)-2-[[2-[4-bromo-3-chloro-5-(hydroxymethyl)anilino]-5-(trifluoromethyl)pyrimidin-4-yl]amino]cyclohexanecarbonitrile (500 mg, 990 umol, 1 eq) in THF (20 mL) was added 2,6-dimethylpyridine (424 mg, 3.96 mmol, 4 eq) and [tert-butyl(dimethyl)silyl] trifluoromethanesulfonate (393 mg, 1.49 mmol, 1.5 eq) at 25° C. The mixture was stirred at 25° C. for 2 hr. TLC showed the reaction was complete. The reaction mixture was quenched by addition of H₂O (20 mL) at 0° C. and extracted with EtOAc (20 mL×3). The combined organic layers were washed with brine (20 mL×2), dried over Na₂SO₄, filtered, and concentrated under reduced pressure to give a residue. The residue was purified by column chromatography (SiO₂, Petroleum ether/ethyl acetate=10/1 to 3/1) to give (trans)-2-((2-((4-bromo-3-(((tert-butyldimethylsilyl)oxy)methyl)-5-chlorophenyl)amino)-5-(trifluoromethyl) pyrimidin-4-yl)amino)cyclohexanecarbonitrile (500 mg, 81.5% yield) as a white solid. ¹H NMR (400 MHz, DMSO-d₆) δ=10.14 (s, 1H), 8.28 (s, 2H), 7.62 (s, 1H), 7.18 (d, J=8.8 Hz, 1H), 4.68 (s, 2H), 4.54-4.38 (m, 1H), 3.19-3.04 (m, 1H), 2.22-2.09 (m, 1H), 1.90-1.80 (m, 1H), 1.79-1.65 (m, 2H), 1.65-1.51 (m, 1H), 1.51-1.40 (m, 1H), 1.40-1.29 (m, 1H), 1.22-1.08 (m, 1H), 0.94 (s, 9H), 0.13 (s, 6H).

### 21.3 Preparation of (trans)-2-[[2-[3-[[tert-butyl(dimethyl)silyl]oxymethyl]-5-chloro-4-(5,5-dimethyl-1,3,2-dioxaborinan-2-yl)anilino]-5-(trifluoromethyl)pyrimidin-4-yl]amino]cyclohexane carbonitrile

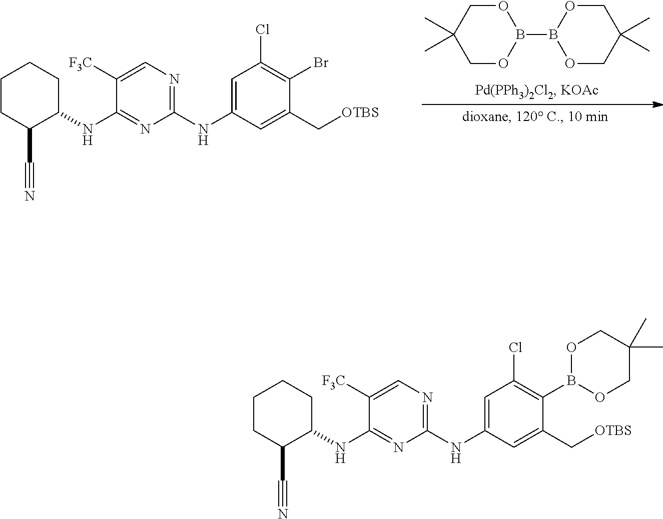

To a solution of (trans)-2-((2-((4-bromo-3-(((tert-butyldimethylsilyl)oxy)methyl)-5-chlorophenyl)amino)-5-(trifluoromethyl)pyrimidin-4-yl)amino)cyclohexanecarbonitrile (400 mg, 646 umol, 1 eq) in dioxane (8 mL) was added Pd(PPh₃)₂Cl₂ (45.3 mg, 64.6 umol, 0.1 eq) and KOAc (190 mg, 1.94 mmol, 3 eq) and 2-(5,5-dimethyl-1,3,2-dioxaborinan-2-yl)-5,5-dimethyl-1,3,2-dioxaborinane (729 mg, 3.23 mmol, 5 eq) at 25° C. The mixture was stirred at 120° C. for 10 min. TLC showed the reaction was complete. The reaction mixture was diluted with H₂O (10 mL) and extracted with EtOAc (20 mL×3). The combined organic layers were washed with brine (10 mL×2), dried over anhydrous Na₂SO₄, filtered, and concentrated under reduced pressure to give a residue. The residue was purified by prep-HPLC (column: Phenomenex luna C18 100*40 mm*3 um; mobile phase: [water (FA formic acid)-ACN]; B %: 30%-80%, 8 min) to give 2-[[2-[3-[[tert-butyl(dimethyl)silyl]oxymethyl]-5-chloro-4-(5,5-dimethyl-1,3,2-dioxaborinan-2-yl)anilino]-5-(trifluoromethyl)pyrimidin-4-yl]amino]cyclohexanecarbonitrile (110 mg, 26.1% yield) as a white solid.

### 21.4 Preparation of (trans)-2-((2-((7-chloro-1-hydroxy-1,3-dihydrobenzo[c][1,2]oxaborol-5-yl)amino)-5-(trifluoromethyl)pyrimidin-4-yl)amino)cyclohexanecarbonitrile

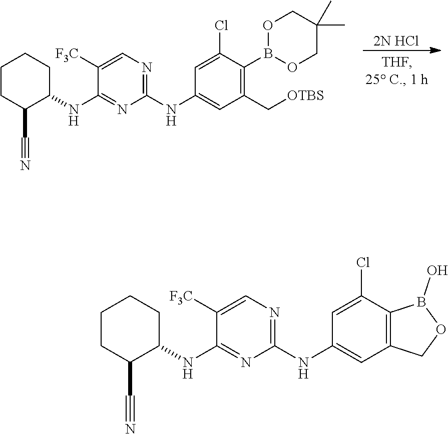

To a solution of (trans)-2-[[2-[3-[[tert-butyl(dimethyl)silyl]oxymethyl]-5-chloro-4-(5,5-dimethyl-1,3,2-dioxaborinan-2-yl)anilino]-5-(trifluoromethyl)pyrimidin-4-yl]amino]cyclohexanecarbonitrile (110 mg, 168 umol, 1 eq) in THF (2 mL) was added aq. HCl (6 M, 2 mL) at 25° C. The mixture was stirred at 25° C. for 1 hr. Solid was precipitated out. TLC showed the reaction was complete. The reaction mixture was filtered and the filter cake was triturated with THE (5 mL) to give (trans)-2-((2-((7-chloro-1-hydroxy-1,3-dihydrobenzo[c][1,2]oxaborol-5-yl)amino)-5-(trifluoromethyl)pyrimidin-4-yl)amino)cyclohexanecarbonitrile (13.6 mg, 17.8% yield) as a white solid. ¹H NMR (400 MHz, DMSO-d₆) δ=10.09 (s, 1H), 8.31 (s, 1H), 7.84 (s, 1H), 7.65 (s, 1H), 7.25 (d, J=8.4 Hz, 1H), 5.03-4.89 (m, 2H), 4.52-4.40 (m, 1H), 3.19-3.05 (m, 1H), 2.25-2.15 (m, 1H), 1.91-1.84 (m, 1H), 1.81-1.67 (m, 2H), 1.67-1.54 (m, 1H), 1.52-1.29 (m, 2H), 1.27-1.06 (m, 1H). MS (ESI): mass calcd. For C₁₉H₁₈BClF₃N₅O₂ 451.12, m/z found 449.9 [M−H]⁻. HPLC: 98.10% (220 nm), 100% (254 nm).

### 22. Preparation of (trans)-2-[[2-[(7-chloro-1-hydroxy-3H-2,1-benzoxaborol-5-yl)amino]-5-(trifluoromethyl)pyrimidin-4-yl]amino]cyclopentanecarbonitrile

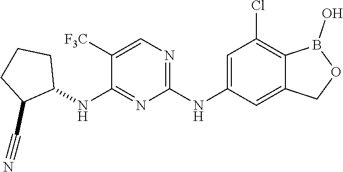 [SMILES: N#C[C@H]1CCC[C@@H]1Nc1nc(Nc2cc(Cl)c3c(c2)COB3O)ncc1C(F)(F)F]

### 22.1 Preparation of (trans)-2-[[2-[4-bromo-3-chloro-5-(hydroxymethyl)anilino]-5-(trifluoromethyl)pyrimidin-4-yl]amino]cyclopentanecarbonitrile

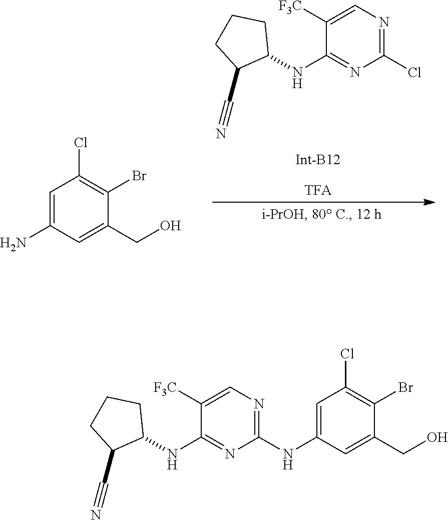

To a solution of (5-amino-2-bromo-3-chloro-phenyl)methanol (500 mg, 2.11 mmol, 1 eq) in i-PrOH (10 mL) was added (trans)-2-[[2-chloro-5-(trifluoromethyl)pyrimidin-4-yl]amino]cyclopentanecarbonitrile (614 mg, 1.83 mmol, 1 eq) and TFA (360 mg, 3.17 mmol, 1.5 eq) at 25° C. The mixture was stirred at 80° C. for 12 h. Solid was precipitate out. TLC showed the reaction was complete. The reaction mixture was filtered to give the crude product (filter cake). The crude product was triturated with saturated aq. NaHCO₃ (30 mL) and filtered to give (trans)-2-[[2-[4-bromo-3-chloro-5-(hydroxymethyl)anilino]-5-(trifluoromethyl)pyrimidin-4-yl]amino]cyclopentanecarbonitrile (820 mg, 79.1% yield) as a white solid. ¹H NMR (DMSO-d₆, 400 MHz) δ 10.16 (s, 1H), 8.33 (s, 1H), 8.02 (s, 1H), 7.94 (s, 1H), 7.42 (d, J=8.0 Hz, 1H), 5.02-4.91 (m, 1H), 4.51 (s, 2H), 3.35-3.20 (m, 1H), 2.23-2.07 (m, 2H), 1.93-1.71 (m, 3H), 1.64-1.52 (m, 1H).

### 22.2 Preparation of (trans)-2-[[2-[4-bromo-3-[[tert-butyl(dimethyl)silyl]oxymethyl]-5-chloro-anilino]-5-(trifluoromethyl)pyrimidin-4-yl]amino]cyclopentanecarbonitrile

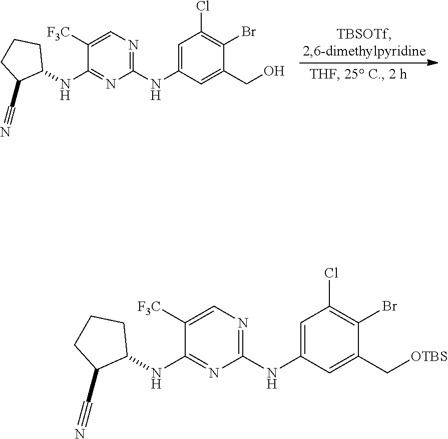

To a solution of (trans)-2-[[2-[4-bromo-3-chloro-5-(hydroxymethyl)anilino]-5-(trifluoromethyl)pyrimidin-4-yl]amino]cyclopentanecarbonitrile (820 mg, 1.67 mmol, 1 eq) in THF (20 mL) was added 2,6-dimethylpyridine (716 mg, 6.68 mmol, 780 uL, 4 eq) and [tert-butyl(dimethyl)silyl] trifluoromethanesulfonate (662 mg, 2.51 mmol, 580 uL, 1.5 eq) at 25° C. The mixture was stirred at 25° C. for 2 h. TLC showed the reaction was complete. The reaction mixture was quenched by addition of H₂O (20 mL) at 0° C. and extracted with EtOAc (10 mL×3). The combined organic layers were washed with brine (20 mL×2), dried over Na₂SO₄, filtered, and concentrated under reduced pressure to give a residue. The residue was purified by column chromatography (SiO₂, petroleum ether/ethyl acetate=10/1 to 5/1) to give (trans)-2-[[2-[4-bromo-3-[[tert-butyl(dimethyl) silyl]oxymethyl]-5-chloro-anilino]-5-(trifluoromethyl)pyrimidin-4-yl]amino] cyclopentanecarbonitrile (840 mg, 83.0% yield) as a white solid. ¹H NMR (DMSO-d₆, 400 MHz) δ 10.14 (s, 1H), 8.29 (s, 2H), 7.66 (s, 1H), 7.28 (d, J=8.0 Hz, 1H), 4.98-4.86 (m, 1H), 4.68 (s, 2H), 3.31-3.24 (m, 1H), 2.26-2.05 (m, 2H), 1.90-1.70 (m, 3H), 1.67-1.54 (m, 1H), 0.94 (s, 9H), 0.13 (s, 6H).

### 22.3 Preparation of (trans)-2-[[2-[3-[[tert-butyl(dimethyl)silyl]oxymethyl]-5-chloro-4-(5,5-dimethyl-1,3,2-dioxaborinan-2-yl)anilino]-5-(trifluoromethyl)pyrimidin-4-yl]amino]cyclopentanecarbonitrile

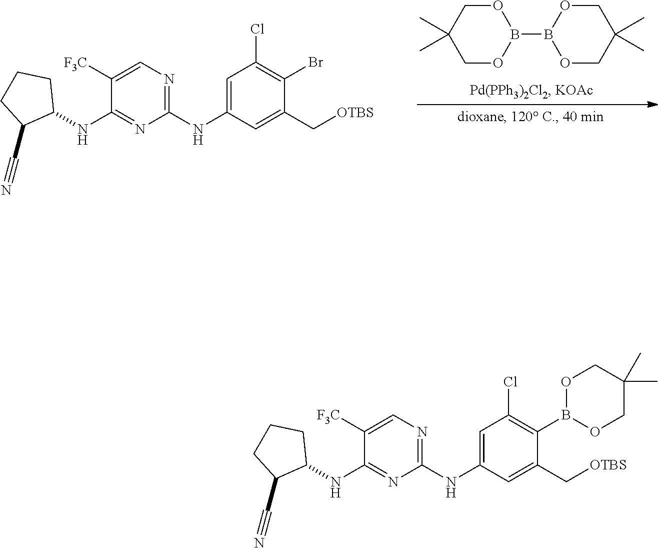

Two reactions were carried out in parallel. For each a mixture of (trans)-2-[[2-[4-bromo-3-[[tert-butyl(dimethyl) silyl]oxymethyl]-5-chloro-anilino]-5-(trifluoromethyl)pyrimidin-4-yl]amino]cyclopentanecarbonitrile (400 mg, 661 umol, 1 eq), 2-(5,5-dimethyl-1,3,2-dioxaborinan-2-yl)-5,5-dimethyl-1,3,2-dioxaborinane (746 mg, 3.31 mmol, 5 eq), Pd(PPh₃)₂Cl₂ (69.6 mg, 99.1 umol, 0.15 eq) and KOAc (142 mg, 1.45 mmol, 2.2 eq) in dioxane (10 mL) was degassed and purged with N₂ for 3 times, and then the mixture was stirred at 120° C. for 40 min under N₂ atmosphere. TLC showed the reaction was complete.

The two parallel reactions were combined, filtered and the filtrate was concentrated under reduced pressure to give a residue. The residue was purified by column chromatography (SiO₂, Petroleum ether/ethyl acetate=3/1 to 1/1) to give (trans)-2-[[2-[3-[[tert-butyl(dimethyl)silyl]oxymethyl]-5-chloro-4-(5,5-dimethyl-1,3,2-dioxaborinan-2-yl)anilino]-5-(trifluoromethyl) pyrimidin-4-yl]amino]cyclopentanecarbonitrile (0.5 g, 59.2% yield) as a yellow solid.

### 22.4 Preparation of (trans)-2-[[2-[(7-chloro-1-hydroxy-3H-2,1-benzoxaborol-5-yl)amino]-5-(trifluoromethyl)pyrimidin-4-yl]amino]cyclopentanecarbonitrile

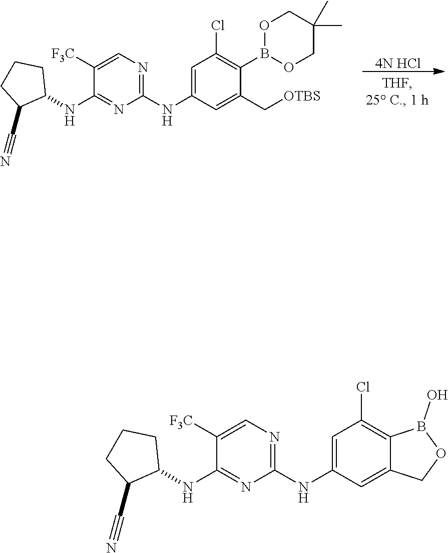

To a solution of (trans)-2-[[2-[3-[[tert-butyl(dimethyl)silyl]oxymethyl]-5-chloro-4-(5,5-dimethyl-1,3,2-dioxaborinan-2-yl) anilino]-5-(trifluoromethyl)pyrimidin-4-yl]amino]cyclopentanecarbonitrile (0.5 g, 783 umol, 1 eq) in THF (5 mL) was added aq. HCl (4 M, 2 mL, 10 eq) at 25° C. The mixture was stirred at 25° C. for 1 h. Solid was precipitated out. TLC showed the reaction was complete. The reaction mixture was filtered to give crude product (filter cake). The crude product was triturated with THF (5 mL) and filtered to give (trans)-2-[[2-[(7-chloro-1-hydroxy-3H-2,1-benzoxaborol-5-yl)amino]-5-(trifluoromethyl)pyrimidin-4-yl]amino]cyclopentanecarbonitrile (157.5 mg, 45.9% yield, 97.36% purity) as a white solid. 1H NMR (DMSO-d₆, 400 MHz) δ 10.10 (s, 1H), 8.33 (s, 1H), 7.83 (s, 1H), 7.72 (s, 1H), 7.33 (d, J=8.4 Hz, 1H), 5.00-4.87 (m, 3H), 3.35-3.25 (m, 1H), 2.26-2.04 (m, 2H), 1.92-1.70 (m, 3H), 1.68-1.55 (m, 1H). MS (ESI): mass calcd. For C₁₈H₁₆BClF₃N₅O₂ 437.10, m/z found 435.9 [M−H]⁻. HPLC: 97.36% (220 nm), 97.17% (254 nm).

### 23. Preparation of (trans)-2-((5-chloro-2-((7 fluoro-1-hydroxy-1,3-dihydrobenzo[c][1,2]oxaborol-5-yl)amino)pyrimidin-4-yl)amino)cyclohexane-1-carbonitrile

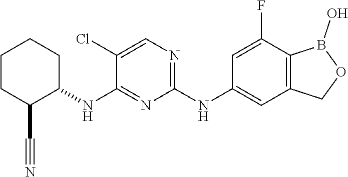

### 23.1 Preparation of (trans)-2-((2-((4-bromo-3 fluoro-5-(hydroxymethyl)phenyl)amino)-5-chloropyrimidin-4-yl)amino)cyclohexanecarbonitrile

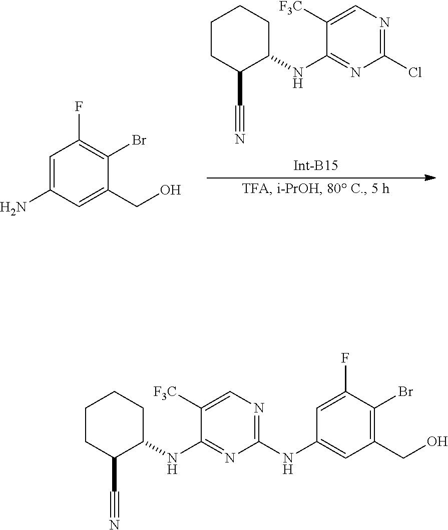

To a solution of (5-amino-2-bromo-3-fluoro-phenyl)methanol (500 mg, 2.27 mmol, 1 eq) in i-PrOH (10 mL) was added TFA (388 mg, 3.41 mmol, 1.5 eq) and (trans)-2-[(2,5-dichloropyrimidin-4-yl)amino]cyclohexanecarbonitrile (616 mg, 2.27 mmol, 1 eq) at 25° C. The mixture was stirred at 80° C. for 5 h. Solid was precipitated out. TLC showed the reaction was complete. The reaction mixture was filtered to obtained filter cake. The filter cake was triturated with saturated aq. NaHCO₃ (10 mL) at 25° C. for 30 min to give (trans)-2-[[2-[4-bromo-3-fluoro-5-(hydroxymethyl)anilino]-5-chloro-pyrimidin-4-yl]amino] cyclohexanecarbonitrile (850 mg, 82.2% yield) as a white solid. ¹H NMR (400 MHz, DMSO-d₆) δ=9.67 (s, 1H), 8.05 (s, 1H), 7.80 (dd, J=2.4, 12.0 Hz, 1H), 7.71 (s, 1H), 7.38 (d, J=9.2 Hz, 1H), 5.52 (s, 1H), 4.50 (s, 2H), 4.40-4.24 (m, 1H), 3.08-2.96 (m 1H), 2.16 (d, J=12.0 Hz, 1H), 1.94-1.86 (m, 1H), 1.76-1.56 (m, 3H), 1.40-1.35 (m, 2H), 1.24-1.09 (m, 1H).

### 23.2 Preparation of (trans)-2-((2-((4-bromo-3-(((tert-butyldimethylsilyl)oxy)methyl)-5-fluorophenyl)amino)-5-chloropyrimidin-4-yl)amino)cyclohexanecarbonitrile

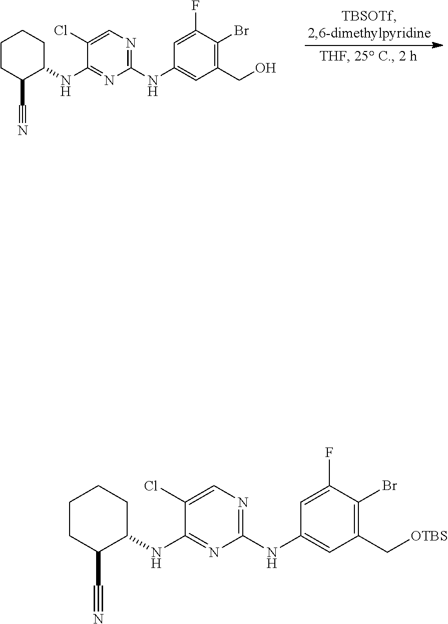

To a solution of (trans)-2-[[2-[4-bromo-3-fluoro-5-(hydroxymethyl)anilino]-5-chloro-pyrimidin-4-yl]amino]cyclohexanecarbonitrile (850 mg, 1.87 mmol, 1 eq) and 2,6-dimethylpyridine (801 mg, 7.48 mmol, 870 uL, 4 eq) in THF (10 mL) was added TBSOTf (741 mg, 2.80 mmol, 640 uL, 1.5 eq) at 25° C. Then the mixture was stirred at 25° C. for 2 h. TLC showed the reaction was complete. The reaction mixture was quenched by addition of saturated aq. NH₄Cl (20 mL) at 0° C., and then extracted with EtOAc (20 mL×3). The combined organic layers were washed with brine (50 mL×2), dried over Na₂SO₄, filtered, and concentrated under reduced pressure to give a residue. The residue was triturated with Petroleum ether (20 mL) at 25° C. for 30 min to give 2-[[2-[4-bromo-3-[[tert-butyl(dimethyl)silyl]oxymethyl]-5-fluoro-anilino]-5-chloro-pyrimidin-4-yl]amino]cyclohexanecarbonitrile (570 mg, 53.5% yield) as a white solid. ¹H NMR (400 MHz, DMSO-d₆) δ=9.81 (s, 1H), 8.03 (s, 1H), 7.99 (dd, J=2.8, 12.4 Hz, 1H), 7.52 (s, 1H), 7.42 (d, J=9.2 Hz, 1H), 4.67 (s, 2H), 4.36-4.20 (m, 1H), 3.09-2.97 (m, 1H), 2.21-2.15 (m, 1H), 1.94-1.87 (m, 1H), 1.81-1.67 (m, 2H), 1.62-1.49 (m, 1H), 1.44-1.25 (m, 2H), 1.25-1.11 (m, 1H), 0.93 (s, 9H), 0.13 (s, 6H).

### 23.3 Preparation of (trans)-2-((2-((3-(((tert-butyldimethylsilyl)oxy)methyl)-4-(5,5-dimethyl-1,3,2-dioxaborinan-2-yl)-5 fluorophenyl)amino)-5-chloropyrimidin-4-yl)amino)cyclohexane-1-carbonitrile

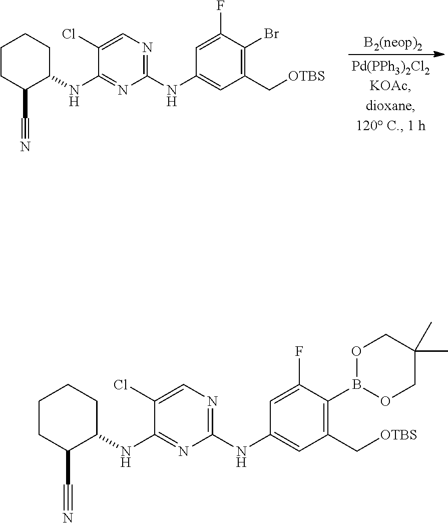

Five reactions were carried out in parallel. For each a mixture of (trans)-2-[[2-[4-bromo-3-[[tert-butyl(dimethyl) silyl]oxymethyl]-5-fluoro-anilino]-5-chloro-pyrimidin-4-yl]amino]cyclohexanecarbonitrile (100 mg, 175 umol, 1 eq), 2-(5,5-dimethyl-1,3,2-dioxaborinan-2-yl)-5,5-dimethyl-1,3,2-dioxaborinane (277 mg, 1.23 mmol, 7 eq), KOAc (51.7 mg, 527 umol, 3 eq), Pd(PPh₃)₂Cl₂ (12.3 mg, 17.5 umol, 0.1 eq) in dioxane (5 mL) was degassed and purged with N₂ for 3 times, and then the mixture was stirred at 120° C. for 1 h under N₂ atmosphere. LCMS showed the reaction was complete and desired MS observed.

The five parallel reactions were combined, filtered, and concentrated under reduced pressure to give a residue. Then the residue was purified by prep-HPLC (column: Phenomenex Luna C18 75*30 mm*3 um; mobile phase: [water (formic acid FA)-ACN]; B %: 40%-70%, 8 min) to give (trans)-2-((2-((3-(((tert-butyldimethylsilyl)oxy)methyl)-4-(5,5-dimethyl-1,3,2-dioxaborinan-2-yl)-5-fluorophenyl)amino)-5-chloropyrimidin-4-yl)amino)cyclohexane-1-carbonitrile (200 mg, 42.6% yield) as a white solid.

### 23.4 Preparation of (trans)-2-((5-chloro-2-((7 fluoro-1-hydroxy-1,3-dihydrobenzo[c][1,2]oxaborol-5-yl)amino)pyrimidin-4-yl)amino)cyclohexane-1-carbonitrile

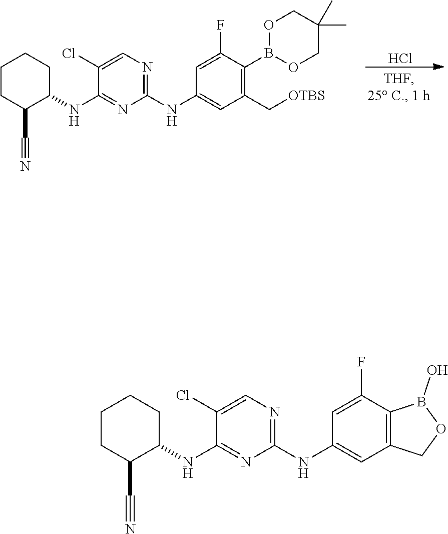

To a solution of (trans)-2-((2-((3-(((tert-butyldimethylsilyl)oxy)methyl)-4-((5-chloro-4-((2-cyanocyclohexyl)amino)pyrimidin-2-yl)amino)-6-fluorophenyl)boronic acid (200 mg, 374 umol, 1 eq) in THF (3 mL) was added aq. HCl (6 M, 0.3 mL, 5 eq) at 25° C. The mixture was stirred at 25° C. for 1 h. Solid was precipitate out. LCMS showed the reaction was complete and desired MS observed. The reaction mixture was filtered to give a residue. The residue was triturated with THF (2 mL) and filtered to give (trans)-2-((5-chloro-2-((7-fluoro-1-hydroxy-1,3-dihydrobenzo[c][1,2]oxaborol-5-yl)amino)pyrimidin-4-yl)amino)cyclohexane-1-carbonitrile (65.5 mg, 43.1% yield) as a white solid. ¹H NMR (400 MHz, DMSO-d₆) δ=9.91 (s, 1H), 8.11 (s, 1H), 7.66 (d, J=7.6 Hz, 1H), 7.54 (s, 1H), 7.51 (d, J=11.2 Hz, 1H), 5.00-4.95 (m, 2H), 4.34-4.22 (m, 1H), 3.10-2.99 (m, 1H), 2.19 (d, J=11.6 Hz, 1H), 1.98-1.89 (m, 1H), 1.82-1.68 (m, 2H), 1.65-1.52 (m, 1H), 1.46-1.26 (m, 2H), 1.25-1.12 (m, 1H). MS (ESI): mass calcd. For C₁₈H₁₈BClFN₅O₂ 401.12, m/z found 402.1 [M+H]⁺. HPLC: 99.09% (220 nm), 99.70% (254 nm).

### 24. Preparation of 2-[[5-chloro-2-[(1-hydroxy-7-methyl-3H-2,1-benzoxaborol-5-yl)amino]pyrimidin-4-yl]amino]cyclohexanecarbonitrile

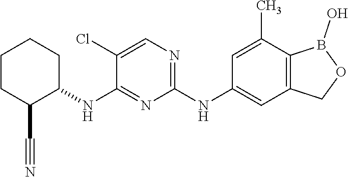

### 24.1 Preparation of 2-bromo-3-methyl-5-nitro-benzoic acid

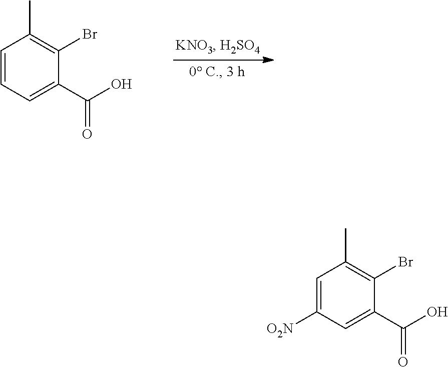

The compound 2-bromo-3-methyl-benzoic acid (10 g, 46.5 mmol, 1 eq) was dissolved in concentrated H₂SO₄ (70 mL). KNO₃ (4.61 g, 45.5 mmol, 0.98 eq) dissolved in H₂SO₄ (30 mL) was drop-wise added to the solution at 0° C. The mixture was stirred for 3 h at 0° C. TLC showed the reaction was complete. The reaction mixture was poured into ice-water (200 mL) and filtered to give 2-bromo-3-methyl-5-nitro-benzoic acid (10.6 g, crude) as a brown solid. ¹H NMR (CDCl₃, 400 MHz) δ 8.55 (d, J=2.8 Hz, 1H), 8.27 (d, J=2.8 Hz, 1H), 2.63 (s, 3H).

### 24.2 Preparation of methyl 2-bromo-3-methyl-5-nitro-benzoate

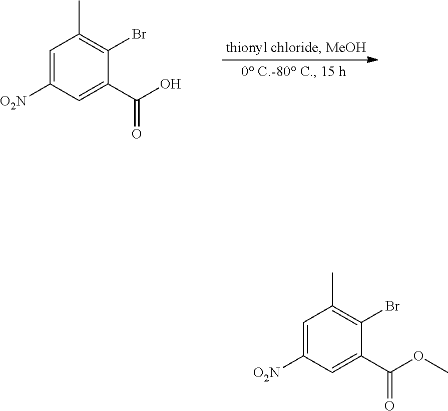

To a mixture of 2-bromo-3-methyl-5-nitro-benzoic acid (8.5 g, 32.7 mmol, 1 eq) in MeOH (100 mL) was drop-wise added thionyl chloride (65.3 mmol, 4.7 mL, 2 eq) at 0° C. The mixture was heated to 80° C. and stirred for 15 h. TLC showed the reaction was complete. The reaction mixture was concentrated in vacuum to give a residue. The residue was purified by column chromatography (SiO₂, petroleum ether/ethyl acetate=10/1 to 5/1) to give methyl 2-bromo-3-methyl-5-nitro-benzoate (8.5 g, 94.9% yield) as an off-white solid. ¹H NMR (CDCl₃, 400 MHz) δ 8.35 (d, J=2.4 Hz, 1H), 8.20 (d, J=2.4 Hz, 1H), 3.99 (s, 3H), 2.59 (s, 3H).

### 24.3 Preparation of methyl 5-amino-2-bromo-3-methyl-benzoate

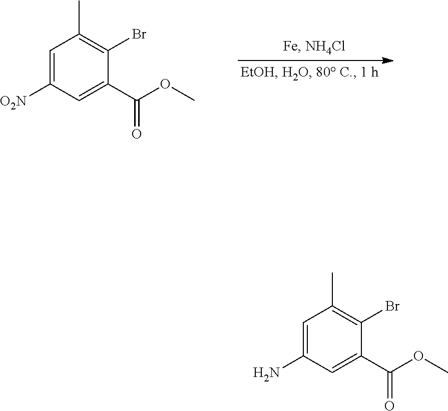

To a solution of methyl 2-bromo-3-methyl-5-nitro-benzoate (7.5 g, 27.3 mmol, 1 eq) in EtOH (100 mL) and H₂O (20 mL) was added NH₄Cl (5.86 g, 109 mmol, 4 eq) and Fe powder (6.11 g, 109 mmol, 4 eq) at 25° C. The mixture was stirred at 80° C. for 1 h. TLC showed the reaction was complete. The reaction mixture was filtered and the filtrate was concentrated under reduced pressure to give a residue. The residue was diluted with H₂O (50 mL) and extracted with EtOAc (50 mL×3). The combined organic layers were washed with brine (50 mL×2), dried over Na₂SO₄, filtered, and concentrated under reduced pressure to give methyl 5-amino-2-bromo-3-methyl-benzoate (5.5 g, 82.3% yield) as brown oil. ¹H NMR (CDCl₃, 400 MHz) δ 6.80 (s, 1H), 6.68 (s, 1H), 3.91 (s, 3H), 3.72 (s, 2H), 2.36 (s, 3H).

### 24.4 Preparation of (5-amino-2-bromo-3-methyl-phenyl)methanol

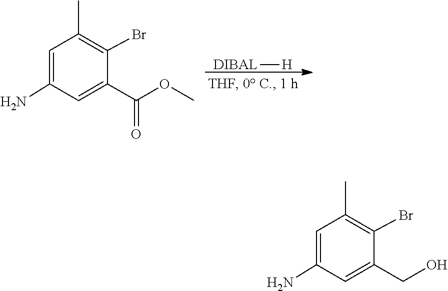

To a solution of methyl 5-amino-2-bromo-3-methyl-benzoate (5.5 g, 22.5 mmol, 1 eq) in THF (50 mL) was added DIBAL-H (1 M in THF, 90.1 mL, 4 eq) drop-wise at 0° C. The mixture was stirred at 0° C. for 1 h. TLC showed the reaction was complete. The reaction mixture was poured into saturated.aq.seignette salt (100 mL) at 0° C. and stirred at room temperature for 1 h, filtered and the filtrate was concentrated under reduced pressure to give a residue. The residue was triturated with MTBE (30 mL) and filtered to give (5-amino-2-bromo-3-methyl-phenyl)methanol (1.5 g, 30.8% yield) as a yellow solid. ¹H NMR (CDCl₃, 400 MHz) δ 6.67 (d, J=2.8 Hz, 1H), 6.54 (d, J=2.8 Hz, 1H), 4.66 (s, 2H), 3.58 (s, 2H), 2.33 (s, 3H).

### 24.5 Preparation of (trans)-2-[[2-[4-bromo-3-(hydroxymethyl)-5-methyl-anilino]-5-chloro-pyrimidin-4-yl]amino]cyclohexanecarbonitrile

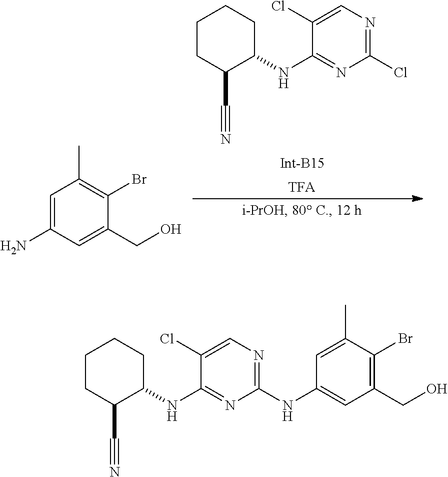 [SMILES: N#C[C@H]1CCCC[C@@H]1Nc1nc(Cl)ncc1Cl ; Cc1cc(N)cc(CO)c1Br ; Cc1cc(Nc2ncc(Cl)c(N[C@H]3CCCC[C@@H]3C#N)n2)cc(CO)c1Br]

To a solution of (5-amino-2-bromo-3-methyl-phenyl)methanol (500 mg, 2.31 mmol, 1 eq) in i-PrOH (5 mL) was added (trans)-2-[(2,5-dichloropyrimidin-4-yl)amino]cyclohexanecarbonitrile (627 mg, 2.31 mmol, 1 eq) and TFA (395 mg, 3.47 mmol, 256 uL, 1.5 eq) at 25° C. The mixture was stirred at 80° C. for 12 h. Solid was precipitated out. TLC showed the reaction was complete. The reaction mixture was filtered to give a residue. The residue was triturated with saturated aq. NaHCO₃ (30 mL) and filtered to give (trans)-2-[[2-[4-bromo-3-(hydroxymethyl)-5-methyl-anilino]-5-chloro-pyrimidin-4-yl]amino]cyclohexanecarbonitrile (875 mg, 83.8% yield) as a yellow solid. ¹H NMR (CDCl₃, 400 MHz) δ 7.95 (s, 1H), 7.67 (d, J=2.8 Hz, 1H), 7.34 (d, J=2.4 Hz, 1H), 7.02 (s, 1H), 5.25 (d, J=8.4 Hz, 1H), 4.76 (s, 2H), 4.49-4.40 (m, 1H), 2.84-2.74 (m, 1H), 2.43 (s, 3H), 2.19-2.06 (m, 2H), 1.85-1.69 (m, 3H), 1.62-1.35 (m, 3H).

### 24.6 Preparation of (trans)-2-[[2-[4-bromo-3-[[tert-butyl(dimethyl)silyl]oxymethyl]-5-methyl-anilino]-5-chloro pyrimidin-4-yl]amino]cyclohexanecarbonitrile

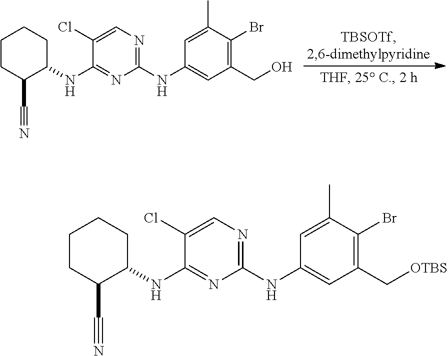 [SMILES: Cc1cc(Nc2ncc(Cl)c(N[C@H]3CCCC[C@@H]3C#N)n2)cc(CO)c1Br ; Cc1cc(Nc2ncc(Cl)c(N[C@H]3CCCC[C@@H]3C#N)n2)cc(CO[Si](C)(C)C(C)(C)C)c1Br]

To a solution of (trans)-2-[[2-[4-bromo-3-(hydroxymethyl)-5-methyl-anilino]-5-chloro-pyrimidin-4-yl]amino]cyclohexanecarbonitrile (460 mg, 1.02 mmol, 1 eq) in THF (5 mL) was added 2,6-dimethylpyridine (437 mg, 4.08 mmol, 4 eq) and [tert-butyl(dimethyl)silyl]trifluoromethanesulfonate (404 mg, 1.53 mmol, 1.5 eq) at 25° C. The mixture was stirred at 25° C. for 2 h. TLC showed the reaction was complete. The reaction mixture was quenched by addition of H₂O (20 mL) at 0° C. and extracted with EtOAc (20 mL×3). The combined organic layers were washed with brine (20 mL×2), dried over Na₂SO₄, filtered, and concentrated under reduced pressure to give a residue. The residue was purified by column chromatography (SiO₂, Petroleum ether/ethyl acetate=10/1 to 5/1) to give (trans)-2-[[2-[4-bromo-3-[[tert-butyl(dimethyl)silyl] oxymethyl]-5-methyl-anilino]-5-chloro-pyrimidin-4-yl]amino]cyclohexanecarbonitrile (500 mg, 86.7% yield) as a yellow solid. ¹H NMR (DMSO-d₆, 400 MHz) δ 9.43 (s, 1H), 7.98 (s, 1H), 7.78 (s, 1H), 7.58 (s, 1H), 7.27 (d, J=9.2 Hz, 1H), 4.66 (s, 2H), 4.35-4.24 (m, 1H), 3.10-3.00 (m, 1H), 2.35 (s, 3H), 2.22-2.13 (m, 1H), 1.92-1.83 (m, 1H), 1.79-1.66 (m, 2H), 1.62-1.49 (m, 1H), 1.37-1.10 (m, 3H), 0.94 (s, 9H), 0.12 (s, 6H).

### 24.7 Preparation of (trans)-2-((2-((3-(((tert-butyldimethylsilyl)oxy)methyl)-4-(5,5-dimethyl-1,3,2-dioxaborinan-2-yl)-5-methylphenyl)amino)-5-chloropyrimidin-4-yl)amino)cyclohexane-1-carbonitrile

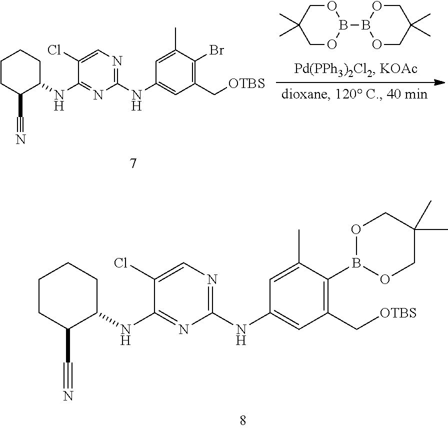

A mixture of (trans)-2-[[2-[4-bromo-3-[[tert-butyl(dimethyl)silyl]oxymethyl]-5-methyl-anilino]-5-chloro-pyrimidin-4-yl]amino]cyclohexanecarbonitrile (500 mg, 884 umol, 1 eq), 2-(5,5-dimethyl-1,3,2-dioxaborinan-2-yl)-5,5-dimethyl-1,3,2-dioxaborinane (999 mg, 4.42 mmol, 5 eq), Pd(PPh₃)₂Cl₂ (93 mg, 132 umol, 0.15 eq) and KOAc (191 mg, 1.95 mmol, 2.2 eq) in dioxane (10 mL) was degassed and purged with N₂ for 3 times, and then the mixture was stirred at 120° C. for 40 min under N₂ atmosphere. TLC showed the reaction was complete. The reaction mixture was filtered and the filtrate was concentrated under reduced pressure to give a residue. The residue was purified by column chromatography (SiO₂, petroleum ether/ethyl acetate=3/1 to 1/1) to give (trans)-2-((2-((3-(((tert-butyldimethylsilyl)oxy)methyl)-4-(5,5-dimethyl-1,3,2-dioxaborinan-2-yl)-5-methylphenyl)amino)-5-chloropyrimidin-4-yl)amino)cyclohexane-1-carbonitrile (0.4 g, 75.5% yield) as a yellow solid.

### 24.8 Preparation of (trans)-2-[[5-chloro-2-[(1-hydroxy-7-methyl-3H-2,1-benzoxaborol-5-yl)amino]pyrimidin-4-yl]amino]cyclohexanecarbonitrile

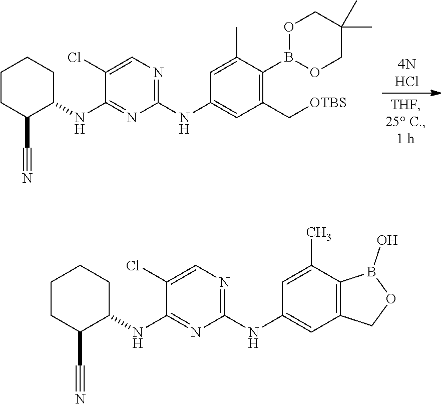

To a solution of (trans)-2-((2-((3-(((tert-butyldimethylsilyl)oxy)methyl)-4-(5,5-dimethyl-1,3,2-dioxaborinan-2-yl)-5-methylphenyl)amino)-5-chloropyrimidin-4-yl)amino)cyclohexane-1-carbonitrile (0.4 g, 668 umol, 1 eq) in THF (5 mL) was added aq. HCl (4 M, 1.67 mL, 10 eq) at 25° C. The mixture was stirred at 25° C. for 1 h. Solid was precipitate out. TLC showed the reaction was complete. The reaction mixture was filtered to give a residue. The residue was triturated with THF (5 mL) and filtered to give (trans)-2-[[5-chloro-2-[(1-hydroxy-7-methyl-3H-2,1-benzoxaborol-5-yl)amino]pyrimidin-4-yl]amino]cyclohexanecarbonitrile (99.0 mg, 37.2% yield, 98.68% purity) as a white solid. ¹H NMR (DMSO-d₆, 400 MHz) δ 9.98 (s, 1H), 8.16 (s, 1H), 8.01 (d, J=4.4 Hz, 1H), 7.55 (s, 1H), 7.34 (s, 1H), 4.99-4.88 (m, 2H), 4.32-4.21 (m, 1H), 3.10-3.00 (m, 1H), 2.42 (s, 3H), 2.23-2.14 (m, 1H), 1.95-1.86 (m, 1H), 1.82-1.67 (m, 2H), 1.64-1.51 (m, 1H), 1.48-1.36 (m, 1H), 1.34-1.10 (m, 2H). MS (ESI): mass calcd. For C₁₉H₂₁BClN₅O₂ 397.15, m/z found 398.2 [M+H]⁺. HPLC: 98.68% (220 nm), 98.78% (254 nm).

### 25. Preparation of 3-[[2-[(7-cyclopropyl-1-hydroxy-3H-2,1-benzoxaborol-5-yl)amino]-5-methyl pyrimidin-4-yl]amino]tetrahydropyran-4-carbonitrile

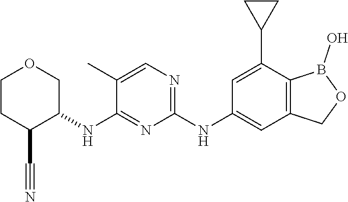

### 25.1 Preparation of methyl 2-hydroxy-3-methyl-benzoate

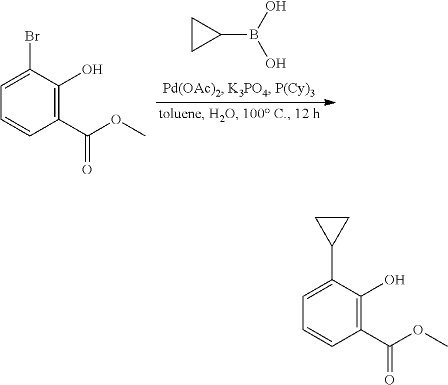

Four reactions were carried out in parallel. For each a solution of methyl 3-bromo-2-hydroxy-benzoate (8.75 g, 37.8 mmol, 1 eq) in toluene (360 mL) and water (36 mL) was added cyclopropylboronic acid (13.0 g, 151 mmol, 4 eq), tricyclohexylphosphane (4.25 g, 15.1 mmol, 4.91 mL, 0.4 eq), K₃PO₄ (16.0 g, 75.7 mmol, 2 eq), and Pd(OAc)₂ (850 mg, 3.79 mmol, 0.1 eq) at 20° C. The mixture was stirred at 100° C. for 12 h. TLC showed the reaction was complete. The four parallel reactions were combined and poured into H₂O (150 mL) and extracted with MTBE (200 mL×3). The combined organic layers were washed with brine (100 mL×3), dried over Na₂SO₄, filtered, and concentrated under reduced pressure to give a residue. The residue was purified by column chromatography (SiO₂, petroleum ether/ethyl acetate=99/1 to 3/1) to give methyl 2-hydroxy-3-methyl-benzoate (11.5 g, 45.6% yield) as yellow oil. ¹H NMR (CDCl₃, 400 MHz) δ 11.17 (s, 1H), 7.67 (d, J=7.6 Hz, 1H), 7.05 (d, J=7.6 Hz, 1H), 6.78 (t, J=8.0 Hz, 1H), 3.95 (s, 3H), 2.25-2.18 (m, 1H), 1.00-0.95 (m, 2H), 0.70-0.65 (m, 2H).

### 25.2 Preparation of methyl 3-cyclopropyl-2-hydroxy-5-nitro-benzoate

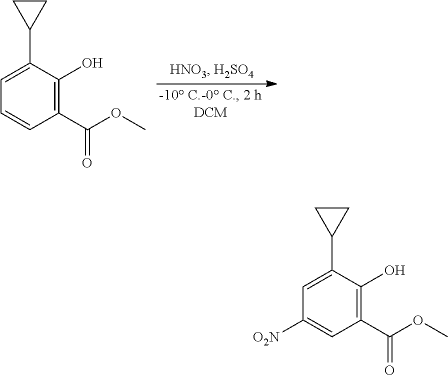 [SMILES: COC(=O)c1cccc(C2CC2)c1O ; COC(=O)c1cc([N+](=O)[O-])cc(C2CC2)c1O]

Two reactions were carried out in parallel. For each a solution of methyl 3-cyclopropyl-2-hydroxy-benzoate (8.4 g, 43.7 mmol, 1 eq) in DCM (35 mL) was cooled to −10° C., then mixed acid of HNO₃ (6.35 g, 65.5 mmol, 4.5 mL, 65% purity, 1.5 eq) and H₂SO₄ (15.4 g, 157 mmol, 8.4 mL, 3.61 eq) was added dropwise at −10° C. After the addition, the reaction was allowed to warm to 0° C. and stirred at 0° C. for 2 hr. TLC showed the reaction was complete. The two parallel reactions were combined and poured into H₂O (100 mL) and extracted with DCM (50 mL×3). The combined organic layers were washed with brine (30 mL×3), dried over Na₂SO₄, filtered, and concentrated under reduced pressure to give a residue. The residue was purified by column chromatography (SiO₂, petroleum ether/ethyl acetate=20/1 to 3/1) to give methyl 3-cyclopropyl-2-hydroxy-5-nitro-benzoate (10.6 g, 51.1% yield) as a yellow solid. ¹H NMR (CDCl₃, 400 MHz) δ 11.85 (s, 1H), 8.62 (s, 1H), 7.91 (s, 1H), 4.04 (s, 3H), 2.28-2.20 (m, 1H), 1.15-1.01 (m, 2H), 0.81-0.74 (m, 2H).

### 25.3 Preparation of methyl 5-amino-3-cyclopropyl-2-hydroxy-benzoate

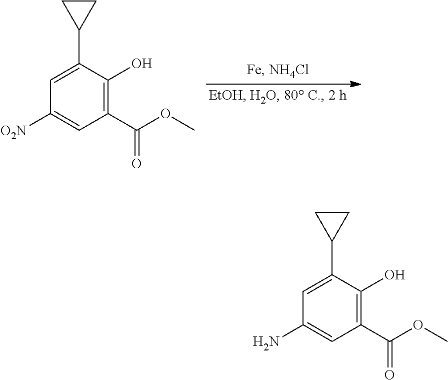 [SMILES: COC(=O)c1cc([N+](=O)[O-])cc(C2CC2)c1O ; COC(=O)c1cc(N)cc(C2CC2)c1O]

To a solution of methyl 3-cyclopropyl-2-hydroxy-5-nitro-benzoate (3.5 g, 14.7 mmol, 1 eq) in EtOH (53 mL) and water (17 mL) was added Fe powder (2.47 g, 44.2 mmol, 3 eq) and NH₄Cl (1.18 g, 22.1 mmol, 1.5 eq) at 25° C. The mixture was stirred at 80° C. for 2 hr. TLC showed the reaction was complete. The reaction mixture was filtered and the filtrate was concentrated to give a residue. The residue was extracted with EtOAc (50 mL×3). The combined organic layers were washed with brine (30 mL×3), dried over Na₂SO₄, filtered, and concentrated under reduced pressure to give residue. The residue was purified by column chromatography (SiO₂, petroleum ether/ethyl acetate=2/1 to 3/1) to give methyl 5-amino-3-cyclopropyl-2-hydroxy-benzoate (2.7 g, 87.2% yield) as a yellow solid. ¹H NMR (CDCl₃, 400 MHz) δ 10.62 (s, 1H), 7.00 (s, 1H), 6.48 (s, 1H), 3.93 (s, 3H), 3.41 (s, 2H), 2.24-2.15 (m, 1H), 1.01-0.90 (m, 2H), 0.70-0.60 (m, 2H).

### 25.4 Preparation of methyl 5-((4-(((trans)-4-cyanotetrahydro-2H-pyran-3-yl)amino)-5-methylpyrimidin-2-yl)amino)-3-cyclopropyl-2-hydroxybenzoate

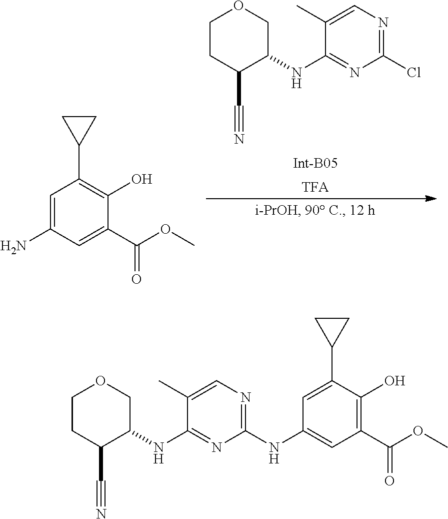 [SMILES: COC(=O)c1cc(N)cc(C2CC2)c1O ; Cc1cnc(Cl)nc1N[C@H]1COCC[C@@H]1C#N ; COC(=O)c1cc(Nc2ncc(C)c(N[C@H]3COCC[C@@H]3C#N)n2)cc(C2CC2)c1O]

To a solution of methyl 5-amino-3-cyclopropyl-2-hydroxy-benzoate (0.85 g, 4.10 mmol, 1 eq) and (trans)-3-[(2-chloro-5-methyl-pyrimidin-4-yl)amino]tetrahydropyran-4-carbonitrile (1.04 g, 4.10 mmol, 1 eq) in i-PrOH (34 mL) was added TFA (935 mg, 8.20 mmol, 2 eq) at 20° C. The mixture was stirred at 90° C. for 12 h. TLC showed the reaction was complete. The residue was poured into H₂O (35 mL) and extracted with EtOAc (30 mL×3). The combined organic layers were washed with brine (20 mL×3), dried over Na₂SO₄, filtered, and concentrated under reduced pressure to give a residue. The residue was purified by column chromatography (SiO₂, petroleum ether/ethyl acetate=2/1 to 0/1) to give methyl 5-((4-(((trans)-4-cyanotetrahydro-2H-pyran-3-yl)amino)-5-methylpyrimidin-2-yl)amino)-3-cyclopropyl-2-hydroxybenzoate (1.25 g, 71.9% yield) as a yellow solid. ¹H NMR (CDCl₃, 400 MHz) δ 11.49 (br s, 1H), 11.11 (s, 1H), 7.86 (s, 1H), 7.49 (s, 1H), 7.05 (s, 1H), 5.99 (d, J=7.2 Hz, 1H), 4.25-4.22 (m, 1H), 4.08-4.03 (m, 1H), 3.96 (s, 3H), 3.86-3.83 (m, 1H), 3.78-3.74 (m, 2H), 3.42-3.30 (m, 1H), 2.13-2.06 (m, 1H), 2.05-2.01 (m, 4H), 1.85-1.79 (m, 1H), 1.01-0.90 (m, 2H), 0.70-0.60 (m, 2H).

### 25.5 Preparation of methyl 5-((4-(((trans)-4-cyanotetrahydropyran-3-yl]amino]-5-methyl pyrimidin-2-yl]amino]-3-cyclopropyl-2-(trifluoromethylsulfonyloxy)benzoate

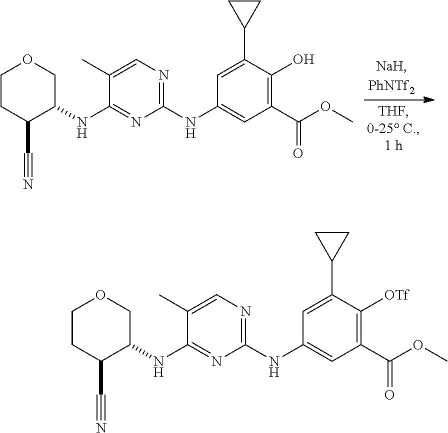 [SMILES: COC(=O)c1cc(Nc2ncc(C)c(N[C@H]3COCC[C@@H]3C#N)n2)cc(C2CC2)c1O ; COC(=O)c1cc(Nc2ncc(C)c(N[C@H]3COCC[C@@H]3C#N)n2)cc(C2CC2)c1OS(=O)(=O)C(F)(F)F]

To a solution of methyl 5-((4-(((trans)-4-cyanotetrahydropyran-3-yl]amino]-5-methyl-pyrimidin-2-yl]amino]-3-cyclopropyl-2-hydroxy-benzoate (1.15 g, 2.72 mmol, 1 eq) in THF (20 mL) was added NaH (325 mg, 8.15 mmol, 60% purity, 3 eq) at 0° C. and stirred for 10 min. Then, 1,1,1-trifluoro-N-phenyl-N-(trifluoromethylsulfonyl)methanesulfonamide (PhNTf₂, 2.91 g, 8.15 mmol, 3 eq) in THF (30 mL) was added to the reaction. The mixture was stirred at 25° C. for 1 hr. TLC showed the reaction was complete. The residue was poured into ice-water (100 mL) and extracted with EtOAC (50 mL×3). The combined organic layers were washed with brine (30 mL×3), dried over Na₂SO₄, filtered, and concentrated under reduced pressure to give a residue. The residue was purified by column chromatography (SiO₂, petroleum ether/ethyl acetate=3/1 to 0/1) to give methyl 5-((4-(((trans)-4-cyanotetrahydropyran-3-yl]amino]-5-methyl-pyrimidin-2-yl]amino]-3-cyclopropyl-2-(trifluoromethylsulfonyloxy)benzoate (1.2 g, 79.5% yield) as a yellow solid. ¹H NMR (CDCl₃, 400 MHz) δ, 8.15 (s, 1H), 7.84 (s, 1H), 7.32 (s, 1H), 7.07 (s, 1H), 5.05 (d, J=7.2 Hz, 1H), 4.45-4.38 (m, 1H), 3.95 (s, 3H), 3.89-3.85 (m, 2H), 3.81-3.78 (m, 1H), 3.32-3.25 (m, 1H), 2.20-2.11 (m, 2H), 2.03 (s, 3H), 1.89-1.81 (m, 1H), 1.15-1.11 (m, 2H), 0.82-0.78 (m, 2H).

### 25.6 Preparation of methyl 5-((4-(((trans)-4-cyanotetrahydropyran-3-yl]amino]-5-methyl pyrimidin-2-yl]amino]-3-cyclopropyl-2-(5,5-dimethyl-1,3,2-dioxaborinan-2-yl)benzoate

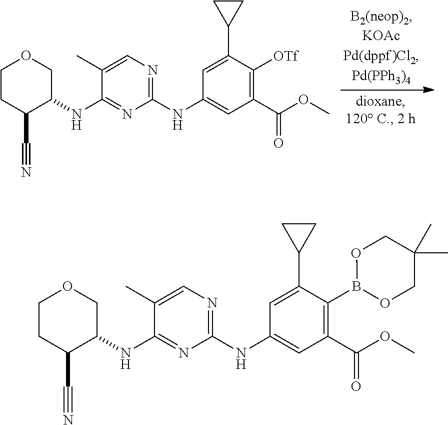 [SMILES: COC(=O)c1cc(Nc2ncc(C)c(N[C@H]3COCC[C@@H]3C#N)n2)cc(C2CC2)c1OS(=O)(=O)C(F)(F)F ; COC(=O)c1cc(Nc2ncc(C)c(N[C@H]3COCC[C@@H]3C#N)n2)cc(C2CC2)c1B1OCC(C)(C)CO1]

A mixture of methyl 5-((4-(((trans)-4-cyanotetrahydropyran-3-yl]amino]-5-methyl-pyrimidin-2-yl]amino]-3-cyclopropyl-2-(trifluoromethylsulfonyloxy)benzoate (0.5 g, 900 umol, 1 eq), 2-(5,5-dimethyl-1,3,2-dioxaborinan-2-yl)-5,5-dimethyl-1,3,2-dioxaborinane (609 mg, 2.70 mmol, 3 eq), KOAc (265 mg, 2.70 mmol, 3 eq), Pd(dppf)Cl₂ (32.9 mg, 45.0 umol, 0.05 eq) and Pd(PPh₃)₄ (52.0 mg, 45.0 umol, 0.05 eq) in dioxane (10 mL) was degassed and purged with N₂ for 3 times. Then the mixture was heated to 120° C. and stirred at 120° C. for 2 h under N₂ atmosphere. TLC showed the reaction was complete. The residue was poured into water (10 mL) and extracted with EtOAC (10 mL×3). The combined organic layers were washed with brine (10 mL×3), dried over Na₂SO₄, filtered, and concentrated under reduced pressure to give a residue. The residue was purified by prep-HPLC (HCl condition) to give methyl 5-((4-(((trans)-4-cyanotetrahydropyran-3-yl]amino]-5-methyl-pyrimidin-2-yl]amino]-3-cyclopropyl-2-(5,5-dimethyl-1,3,2-dioxaborinan-2-yl)benzoate (160 mg, 34.2% yield) as a yellow solid.

### 25.7 Preparation of (trans)-3-[[2-[(7-cyclopropyl-1-hydroxy-3H-2,1-benzoxaborol-5-yl)amino]-5-methyl pyrimidin-4-yl]amino]tetrahydropyran-4-carbonitrile

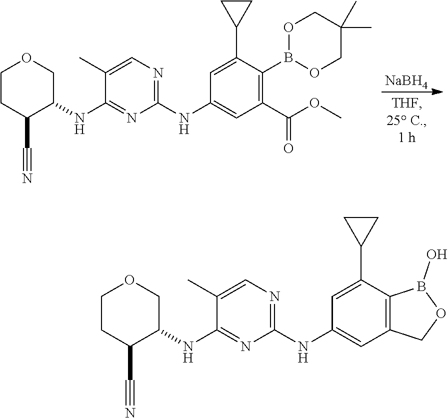 [SMILES: COC(=O)c1cc(Nc2ncc(C)c(N[C@H]3COCC[C@@H]3C#N)n2)cc(C2CC2)c1B1OCC(C)(C)CO1 ; "NaBH4" ; Cc1cnc(Nc2cc3c(c(C4CC4)c2)B(O)OC3)nc1N[C@H]1COCC[C@@H]1C#N]

To a solution of methyl 5-((4-(((trans)-4-cyanotetrahydropyran-3-yl)amino]-5-methyl-pyrimidin-2-yl]amino]-3-cyclopropyl-2-(5,5-dimethyl-1,3,2-dioxaborinan-2-yl)benzoate (0.13 g, 250 umol, 1 eq) in THF (2 mL) was added NaBH₄ (28.4 mg, 750 umol, 3 eq) at 0° C. The mixture was stirred at 25° C. for 1 h. TLC showed the reaction was complete. The reaction mixture was quenched by addition H₂O (2 mL) at 0° C. and adjust pH=6 with aq. HCl (2N), then filtered to give (trans)-3-[[2-[(7-cyclopropyl-1-hydroxy-3H-2,1-benzoxaborol-5-yl)amino]-5-methyl-pyrimidin-4-yl]amino]tetrahydropyran-4-carbonitrile (70.8 mg, 69.8% yield, 96.9% purity) as a white solid. ¹H NMR (DMSO-d₆, 400 MHz) δ 8.91 (s, 1H), 8.23 (s, 1H), 7.83 (s, 1H), 7.62 (d, J=8.8 Hz, 1H), 7.53 (s, 1H), 6.87 (s, 1H), 4.99-4.88 (m, 2H), 4.39-4.28 (m, 1H), 3.92-3.81 (m, 2H), 3.31-3.19 (m, 2H), 3.14 (t, J=10.4 Hz, 1H), 2.48-2.41 (m, 1H), 2.22-2.14 (m, 1H), 1.99 (s, 3H), 1.93-1.80 (m, 1H), 1.03-0.95 (m, 2H), 0.87-0.77 (m, 2H). MS (ESI): mass calcd. For C₂₁H₂₄BN₅O₃ 405.20, m/z found 406.2 [M+H]⁺. HPLC: 96.90% (220 nm), 96.88% (254 nm).

### 26. Preparation of (trans)-3-[[2-[(1-hydroxy-3H-2,1-benzoxaborol-5-yl)amino]-5-methyl-pyrimidin-4-yl]amino]tetrahydropyran-4-carbonitrile

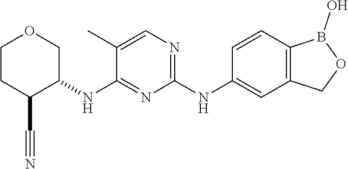

### 26.1 Preparation of (5-amino-2-bromophenyl)methanol

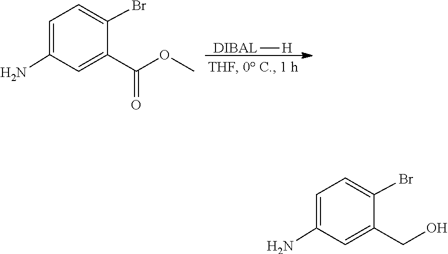

To a solution of methyl 5-amino-2-bromo-benzoate (5 g, 21.7 mmol, 1 eq) in THF (50 mL) was drop-wise added DIBAL-H (1 M in THF, 86.9 mL, 4 eq) at 0° C., and then stirred at 0° C. for 1 h. TLC showed the reaction was complete. The reaction mixture was quenched with saturated aq. Na₂CO₃ (200 mL) and extracted with EtOAc (100 mL×3). The combined organic layers were washed with brine (100 mL), dried over anhydrous Na₂SO₄, filtered, and concentrated under reduced pressure to give a residue. The residue was purified by silica gel column chromatography (petroleum ether:EtOAc=100:1 to 1:1) to obtain (5-amino-2-bromophenyl) methanol (4.3 g) as a yellow solid.

### 26.2 Preparation of (trans)-3-((2-((4-bromo-3-(((tert-butyldimethylsilyl)oxy)methyl) phenyl)amino)-5-methylpyrimidin-4-yl)amino)tetrahydro-2H-pyran-4-carbonitrile

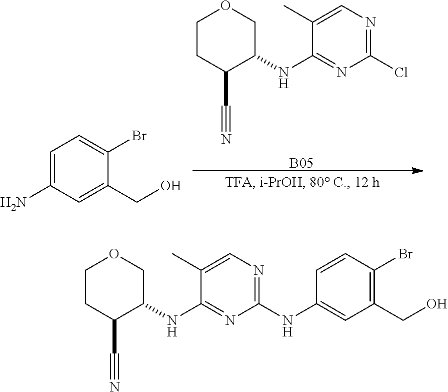 [SMILES: Nc1ccc(Br)c(CO)c1 ; Cc1cnc(Cl)nc1N[C@H]1COCC[C@@H]1C#N ; "B05" ; Cc1cnc(Nc2ccc(Br)c(CO)c2)nc1N[C@H]1COCC[C@@H]1C#N]

A mixture of (5-amino-2-bromo-phenyl)methanol (2 g, 9.90 mmol, 1 eq), (trans)-3-[(2-chloro-5-methyl-pyrimidin-4-yl)amino]tetrahydropyran-4-carbonitrile (2.5 g, 9.90 mmol, 1 eq) and TFA (1.69 g, 14.8 mmol, 1.5 eq) in i-PrOH (40 mL) was degassed and purged with N₂ for 3 times, and then the mixture was stirred at 80° C. for 12 h under N₂ atmosphere. TLC showed the reaction was complete. The reaction mixture was concentrated under reduced pressure to give a residue. The residue was triturated with MTBE (20 mL) to give (trans)-3-((2-((4-bromo-3-(((tert-butyldimethylsilyl)oxy)methyl)phenyl)amino)-5-methylpyrimidin-4-yl)amino)tetrahydro-2H-pyran-4-carbonitrile (5 g, crude) as yellow oil.

### 26.3 Preparation of (trans)-3-((2-((4-bromo-3-(((tert-butyldimethylsilyl)oxy)methyl) phenyl)amino)-5-methylpyrimidin-4-yl)amino)tetrahydro-2H-pyran-4-carbonitrile

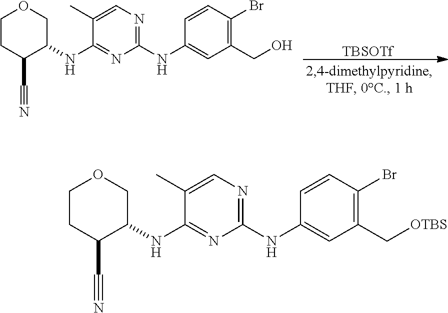 [SMILES: "TBSOTf" ; Cc1cnc(Nc2ccc(Br)c(CO)c2)nc1N[C@H]1COCC[C@@H]1C#N ; Cc1cnc(Nc2ccc(Br)c(CO[Si](C)(C)C(C)(C)C)c2)nc1N[C@H]1COCC[C@@H]1C#N]

A mixture of (trans)-3-[[2-[4-bromo-3-(hydroxymethyl)anilino]-5-methyl-pyrimidin-4-yl]amino]tetrahydropyran-4-carbonitrile (4.9 g, 11.7 mmol, 1 eq), and 2,4-dimethylpyridine (5 g, 46.8 mmol, 4 eq) in THE (40 mL) was added [tert-butyl(dimethyl)silyl]trifluoromethanesulfonate (6.2 g, 23.4 mmol, 2 eq) under N₂, and then the mixture was stirred at 0° C. for 1 h. TLC showed the reaction was complete. The reaction mixture was quenched with aq. NH₄Cl (100 mL) and extracted with EtOAc (100 mL×3). The combined organic layers were washed with brine (100 mL), dried over anhydrous Na₂SO₄, filtered, and concentrated under reduced pressure to give a residue. The residue was purified by silica gel column chromatography (petroleum ether:EtOAc=100:1 to 1:1) to give (trans)-3-((2-((4-bromo-3-(((tert-butyldimethylsilyl)oxy)methyl)phenyl)amino)-5-methylpyrimidin-4-yl)amino)tetrahydro-2H-pyran-4-carbonitrile (5 g, 80% yield) as yellow oil. ¹H NMR (400 MHz, DMSO-d6) δ 9.18 (s, 1H), 7.97 (d, J=2.4 Hz, 1H), 7.73 (s, 1H), 7.64 (dd, J=2.8, 8.8 Hz, 1H), 7.37 (d, J=8.8 Hz, 1H), 6.66 (d, J=8.4 Hz, 1H), 4.64 (s, 2H), 4.55-4.35 (m, 1H), 3.87 (m, 2H), 3.30-3.19 (m, 2H), 3.13 (t, J=10.8 Hz, 1H), 2.22-2.10 (m, 1H), 1.91-1.77 (m, 1H), 0.94 (s, 9H), 0.12 (s, 6H).

### 26.4 Preparation of (trans)-3-((2-((3-(((tert-butyldimethylsilyl)oxy)methyl)-4-(5,5-dimethyl-1,3,2-dioxaborinan-2-yl)phenyl)amino)-5-methylpyrimidin-4-yl)amino)tetrahydro-2H-pyran-4-carbonitrile

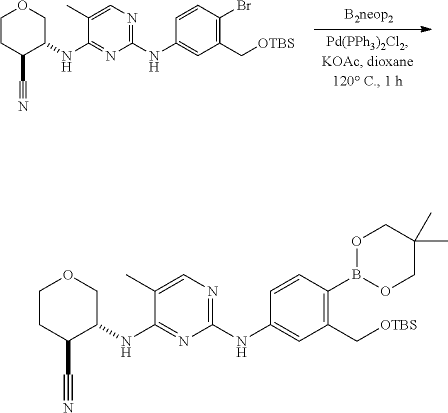

Five reactions were carried out in parallel. For each a mixture of (trans)-3-[[2-[4-bromo-3-[[tert-butyl(dimethyl)silyl]oxymethyl]anilino]-5-methyl-pyrimidin-4-yl]amino]tetrahydropyran-4-carbonitrile (1 g, 1.88 mmol, 1 eq), 2-(5,5-dimethyl-1,3,2-dioxaborinan-2-yl)-5,5-dimethyl-1,3,2-dioxaborinane (1.70 g, 7.51 mmol, 4 eq), KOAc (552 mg, 5.63 mmol, 3 eq) and Pd(PPh₃)₂Cl₂ (131 mg, 187 umol, 0.1 eq) in dioxane (20 mL) was degassed and purged with N₂ for 3 times, and then the mixture was stirred at 120° C. for 1 hr under N₂ atmosphere. TLC showed the reaction was complete. The five parallel reactions were combined and concentrated under reduced pressure to give a residue. The residue was purified by prep-HPLC (column: C18 (250*50 mm*10 um); mobile phase: [water (NH₄HCO₃)-ACN]; B %: 40%-70%, 10 min) to give (trans)-3-((2-((3-(((tert-butyldimethylsilyl)oxy)methyl)-4-(5,5-dimethyl-1,3,2-dioxaborinan-2-yl)phenyl)amino)-5-methylpyrimidin-4-yl)amino)tetrahydro-2H-pyran-4-carbonitrile (5 g, crude) as yellow oil.

### 26.5 Preparation of (3R,4S)-3-((2-((1-hydroxy-1,3-dihydrobenzo[c][1,2]oxaborol-5-yl)amino)-5-methylpyrimidin-4-yl)amino)tetrahydro-2H-pyran-4-carbonitrile

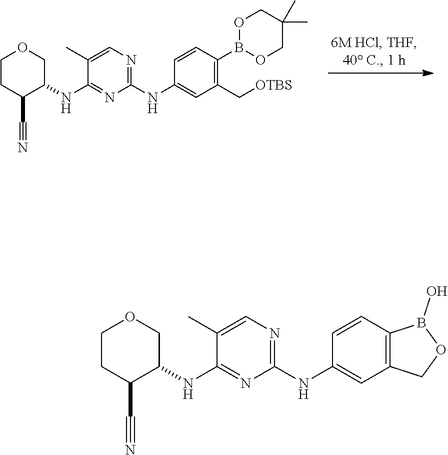

A mixture of (trans)-3-[[2-[3-[[tert-butyl(dimethyl)silyl]oxymethyl]-4-(5,5-dimethyl-1,3,2-dioxaborinan-2-yl)anilino]-5-methyl-pyrimidin-4-yl]amino]tetrahydropyran-4-carbonitrile (4.5 g, 7.96 mmol, 1 eq) in THF (50 mL) was added aq. HCl (6 M, 5.3 mL, 4 eq), and then the mixture was stirred at 40° C. for 1 h under N₂ atmosphere. LCMS showed the reaction was complete and desired MS observed. The reaction mixture was filtered and the filter cake was washed with MeCN (10 mL), dried in vacuum to give (trans)-3-((2-((1-hydroxy-1,3-dihydrobenzo[c][1,2] oxaborol-5-yl)amino)-5-methylpyrimidin-4-yl)amino)tetrahydro-2H-pyran-4-carbonitrile (1.5 g, 51% yield) as a white solid, which was separated by SFC (column: DAICEL CHIRALPAK AD (250 mm*30 mm, 10 um); mobile phase: [0.1% NH₃H₂O EtOH]; B %: 60%-60%, 6 min) to give (trans)-3-((2-((1-hydroxy-1,3-dihydrobenzo[c][1,2]oxaborol-5-yl)amino)-5-methylpyrimidin-4-yl)amino)tetrahydro-2H-pyran-4-carbonitrile (343 mg, 99.34% purity, 100% ee, first peak, RT=1.424 min) stereoisomer one as a white solid. ¹H NMR (400 MHz, DMSO-d₆) δ=9.28 (s, 1H), 8.90 (s, 1H), 7.96 (s, 1H), 7.79 (d, J=0.8 Hz, 1H), 7.61-7.50 (m, 2H), 6.83 (m, 1H), 4.99-4.87 (m, 2H), 4.50-4.40 (m, 1H), 3.95-3.84 (m, 2H), 3.31-3.19 (m, 2H), 3.14 (t, J=10.8 Hz, 1H), 2.23-2.14 (m, 1H), 1.96 (s, 3H), 1.91-1.78 (m, 1H) MS (ESI): mass calcd. For C₁₈H₂₀BN₅O₃, 365.17 m/z found 364.0[M−H]⁻. HPLC: 99.34% (220 nm), 99.9% (254 nm) and (trans)-3-((2-((1-hydroxy-1,3-dihydrobenzo[c][1,2]oxaborol-5-yl)amino)-5-methylpyrimidin-4-yl)amino)tetrahydro-2H-pyran-4-carbonitrile (185 mg, 99.40% purity, 99.90% ee, second peak, RT=1.737 min) stereoisomer two as a white solid. ¹H NMR (400 MHz, DMSO-d₆) δ=9.40 (s, 1H), 8.93 (s, 1H), 7.93 (s, 1H), 7.79 (s, 1H), 7.61-7.55 (m, 1H), 7.54-7.49 (m, 1H), 6.99 (m, 1H), 4.99-4.86 (m, 2H), 4.52-4.39 (m, 1H), 3.90 (m, 2H), 3.29-3.20 (m, 2H), 3.15 (t, J=10.8 Hz, 1H), 2.18 (m, 1H), 1.97 (s, 3H), 1.92-1.79 (m, 1H) MS (ESI): mass calcd. For C₁₈H₂₀BN₅O₃, 365.17 m/z found 364.1[M−H]⁻. HPLC: 99.40% (220 nm), 99.95% (254 nm).

### 27. Preparation of (trans)-3-((2-((1-hydroxy-7-methyl-1,3-dihydrobenzo[c][1,2]oxaborol-5-yl)amino)-5-methylpyrimidin-4-yl)amino)tetrahydro-2H-pyran-4-carbonitrile

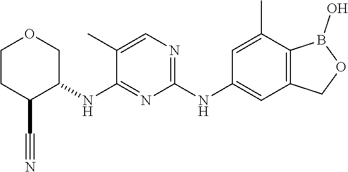

### 27.1 Preparation of methyl 2-bromo-5-((4-(((trans)-4-cyanotetrahydro-2H-pyran-3-yl)amino)-5-methylpyrimidin-2-yl)amino)-3-methylbenzoate

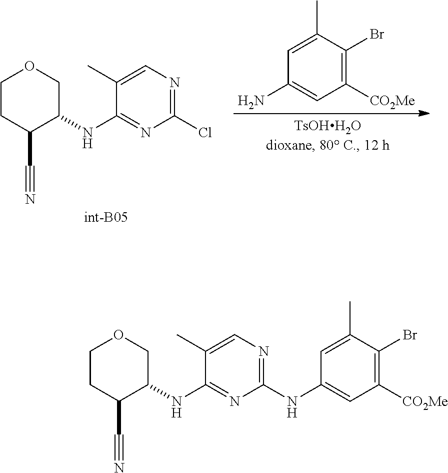

To a solution of (trans)-3-[(2-chloro-5-methyl-pyrimidin-4-yl)amino]tetrahydropyran-4-carbonitrile (700 mg, 2.49 mmol, 90% purity, 1 eq) in dioxane (10 mL) was added TsOH·H₂O (711 mg, 3.74 mmol, 1.5 eq) and methyl 5-amino-2-bromo-3-methyl-benzoate (730 mg, 2.99 mmol, 1.2 eq). The mixture was stirred at 80° C. for 12 h. TLC showed the reaction was complete. The reaction mixture was quenched by addition H₂O (30 mL) at 0° C. and adjust pH=7, then extracted with EtOAc (10 mL×3). The combined organic layers were washed with brine (10 mL×2), dried over with Na₂SO₄, filtered, and concentrated under reduced pressure to give a residue. The residue was purified by flash silica gel chromatography (ISCO®; 20 g SepaFlash® Silica Flash Column, eluent of 100˜0% ethyl acetate/petroleum ether gradient @ 50 mL/min) to give methyl 2-bromo-5-((4-(((trans)-4-cyanotetrahydro-2H-pyran-3-yl)amino)-5-methylpyrimidin-2-yl)amino)-3-methylbenzoate (950 mg, 82% yield) as a yellow solid. ¹H NMR (DMSO-d₆, 400 MHz) δ 7.82 (s, 1H), 7.79 (d, J=2.8 Hz, 1H), 7.64 (d, J=2.4 Hz, 1H), 6.96 (s, 1H), 5.06-5.04 (m, 1H), 4.39-4.37 (m, 1H), 4.16-4.10 (m, 1H), 3.95 (s, 3H), 3.89-3.86 (m, 2H), 3.82-3.79 (m, 1H), 3.32-3.29 (m, 1H), 2.48 (s, 3H), 2.18-2.11 (m, 1H), 2.02 (s, 3H), 1.86-1.82 (m, 1H).

### 27.2 Preparation of methyl 5-[[4-[(4-cyanotetrahydropyran-3-yl)amino]-5-methyl-pyrimidin-2-yl]amino]-2-(5,5-dimethyl-1,3,2-dioxaborinan-2-yl)-3-methyl-benzoate

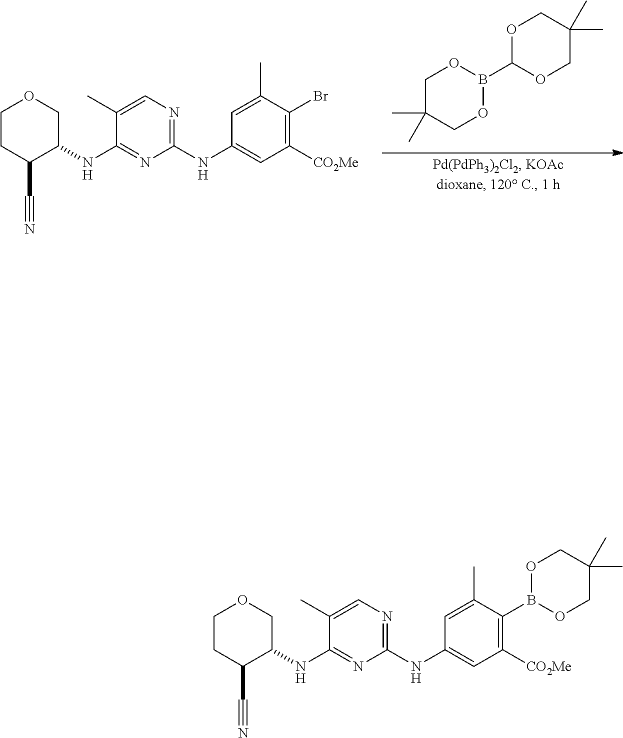

Nine reactions were carried out in parallel. For each a mixture of 2-(5,5-dimethyl-1,3,2-dioxaborinan-2-yl)-5,5-dimethyl-1,3,2-dioxaborinane (123 mg, 543 umol, 2.5 eq), methyl 2-bromo-5-[[4-[(4-cyanotetrahydropyran-3-yl)amino]-5-methyl-pyrimidin-2-yl]amino]-3-methyl-benzoate (100 mg, 217 umol, 1 eq), KOAc (53 mg, 543 umol, 2.5 eq) and Pd(PPh₃)₂Cl₂ (15 mg, 21.7 umol, 0.1 eq) in dioxane (5 mL) was stirred at 120° C. for 1 h under N₂ atmosphere. The nine parallel reactions were combined and the mixture was filtered and concentrated under reduced pressure to give a residue. The residue was purified by flash silica gel chromatography (ISCO®; 20 g SepaFlash® Silica Flash Column, eluent of 0˜50% ethyl acetate/petroleum ether gradient @ 50 mL/min) to give methyl 5-[[4-[((trans)-4-cyanotetrahydropyran-3-yl)amino]-5-methyl-pyrimidin-2-yl)amino]-2-(5,5-dimethyl-1,3,2-dioxaborinan-2-yl)-3-methyl-benzoate (1.1 g, crude) as a brown solid, which was used directly for next step.

### 27.4 Preparation of (trans)-3-((2-((1-hydroxy-7-methyl-1,3-dihydrobenzo[c][1,2] oxaborol-5-yl)amino)-5-methylpyrimidin-4-yl)amino)tetrahydro-2H-pyran-4-carbonitrile

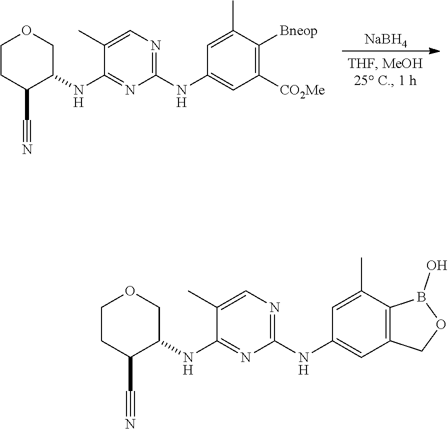

To a solution of methyl 5-[[4-[((trans)4-cyanotetrahydropyran-3-yl)amino]-5-methyl-pyrimidin-2-yl]amino]-2-(5,5-dimethyl-1,3,2-dioxaborinan-2-yl)-3-methyl-benzoate (1 g, 2.03 mmol, 1 eq) in THF (10 mL) and MeOH (2 mL) was added NaBH₄ (767 mg, 20.2 mmol, 10 eq) in portions at 0° C. After the addition, the reaction was allowed to warm to 25° C. and stirred for 1 h. The mixture was adjusted pH=4 with aq. HCl (2N), added H₂O (30 mL) and then extracted with EtOAc (30 mL×3). The combined organic phases were washed with brine (10 mL×3), dried with anhydrous Na₂SO₄, filtered, and concentrated in vacuum to give the residue. The residue was purified by prep-HPLC (column: Phenomenex Luna 80*30 mm*3 um; mobile phase: [water (0.04% HCl)-MEOH]; B %: 10%-30%, 8 min) to give (trans)-3-((2-((1-hydroxy-7-methyl-1,3-dihydrobenzo[c][1,2]oxaborol-5-yl)amino)-5-methylpyrimidin-4-yl)amino)tetrahydro-2H-pyran-4-carbonitrile (127.4 mg, 16% yield) as a white solid. ¹H NMR (DMSO-d₆, 400 MHz) δ 10.16 (s, 1H), 8.86 (s, 1H), 8.10 (s, 1H), 7.82 (s, 1H), 7.44 (s, 1H), 7.31 (s, 1H), 4.94-4.93 (m, 2H), 4.43-4.38 (m, 1H), 4.91-3.85 (m, 2H), 3.23-3.14 (m, 3H), 2.44 (s, 3H), 2.20-2.13 (m, 1H), 2.02 (s, 3H), 1.87-1.83 (m, 1H). MS (ESI): mass calcd. For C₁₉H₂₂BN₅O₃ 379.22, m/z found 380.2 [M+H]⁺. HPLC: 92.4% (220 nm), 89.52% (254 nm).

### 28. Preparation of (trans)-3-[[2-[(1-hydroxy-3-methyl-3H-2,1-benzoxaborol-5-yl)amino]-5-methyl-pyrimidin-4-yl]amino]tetrahydropyran-4-carbonitrile

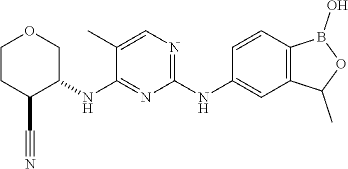

### 28.1 Preparation of (trans)-3-((2-((3-acetyl-4-bromophenyl)amino-5-methylpyrimidi-4-yo)amino)tetrahydro-2H-pyran-4-carbonitrile

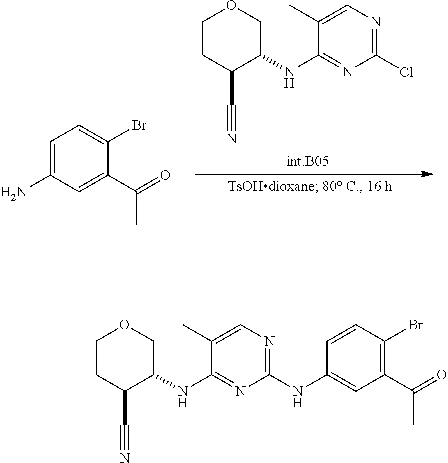

Two reactions were carried out in parallel. For each a solution of 1-(5-amino-2-bromo-phenyl)ethanone (212 mg, 989 umol, 1 eq) and (trans)-3-[(2-chloro-5-methyl-pyrimidin-4-yl)amino]tetrahydropyran-4-carbonitrile (250 mg, 989 umol, 1 eq) in dioxane (3 mL) was added TsOH (256 mg, 1.48 mmol, 1.5 eq) at 20° C. The mixture was heated and stirred at 80° C. for 16 h. TLC showed the reaction was complete. The two parallel reactions were combined and concentrated under reduced pressure to give a residue. The residue was diluted with EtOAc (10 mL) and adjusted pH to 7 with saturated aq. NaHCO₃. The resulting mixture was extracted with EtOAc (10 mL×3). The combined organic layers were washed with brine (15 mL), dried over Na₂SO₄, filtered, and concentrated under reduced pressure to give a residue. The residue was purified by flash silica gel chromatography (ISCO®; 12 g SepaFlash® Silica Flash Column, eluent of 0˜100% ethyl acetate/petroleum ether gradient @ 36 mL/min) to give (trans)-3-[[2-(3-acetyl-4-bromo-anilino)-5-methyl-pyrimidin-4-yl]amino] tetrahydropyran-4-carbonitrile (450 mg) as a yellow solid. ¹H NMR (DMSO-d₆, 400 MHz) δ 9.30 (s, 1H), 8.11 (s, 1H), 7.80 (s, 1H), 7.70 (d, J=8.8 Hz, 1H), 7.51 (d, J=8.8 Hz, 1H), 6.74 (d, J=8.4 Hz, 1H), 4.50-4.40 (m, 1H), 3.90-3.80 (m, 2H), 3.30-3.10 (m, 2H), 2.55 (s, 3H), 2.17 (q, J=10.4 Hz, 1H), 2.20-1.80 (m, 5H).

### 28.2 Preparation of (trans)-3-[[2-[4-bromo-3-(1-hydroxyethyl)anilino]-5-methyl-pyrimidin-4-yl]amino]tetrahydropyran-4-carbonitrile

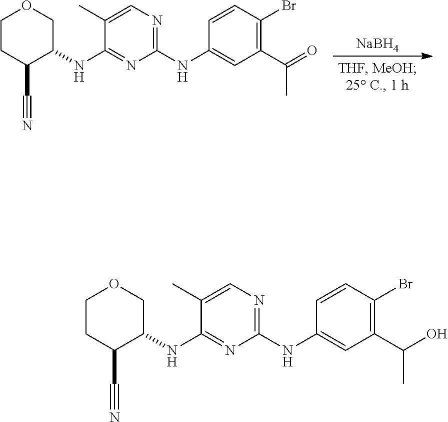

To a solution of (trans)-3-[[2-(3-acetyl-4-bromo-anilino)-5-methyl-pyrimidin-4-yl]amino]tetrahydropyran-4-carbonitrile (300 mg, 697 umol, 1 eq) in THF (3 mL) was added NaBH₄ (380 mg, 10.0 mmol, 14 eq) and MeOH (0.3 mL) at 0° C. After the addition, the reaction was allowed to warm to 25° C. and stirred for 1 h. The reaction mixture was added saturated aq. NH₄Cl (10 mL) at 0° C. and adjust pH to 5 with aq. HCl (2N), and then extracted with EtOAc (10 mL×3). The combined organic layers were washed with brine (15 mL×2), dried over Na₂SO₄, filtered, and concentrated under reduced pressure to give 3-[[2-[4-bromo-3-(1-hydroxyethyl)anilino]-5-methyl-pyrimidin-4-yl] amino]tetrahydropyran-4-carbonitrile (200 mg, 66.3% yield) as a brown solid.

### 28.3 Preparation of (trans)-3-[[2-[(1-hydroxy-3-methyl-3H-2,1-benzoxaborol-5-yl)amino]-5-methyl-pyrimidin-4-yl]amino]tetrahydropyran-4-carbonitrile

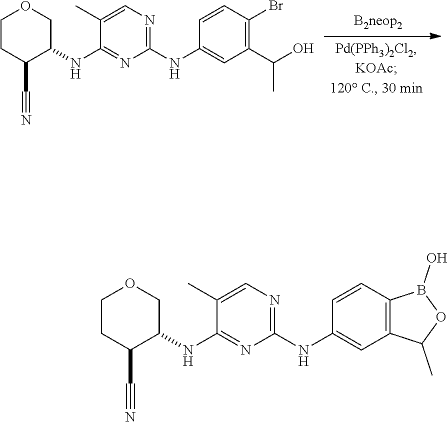

Four reactions were carried out in parallel. For each a mixture of (trans)-3-[[2-[4-bromo-3-(1-hydroxyethyl)anilino]-5-methyl-pyrimidin-4-yl]amino] tetrahydropyran-4-carbonitrile (50 mg, 115 umol, 1 eq), 2-(5,5-dimethyl-1,3,2-dioxaborinan-2-yl)-5,5-dimethyl-1,3,2-dioxaborinane (52 mg, 231 umol, 2 eq), Pd(PPh₃)₂Cl₂ (16 mg, 23.1 umol, 0.2 eq), KOAc (28 mg, 289 umol, 2.5 eq) in dioxane (2 mL) was degassed and purged with N₂ for 3 times, and then the mixture was stirred at 120° C. for 30 min under N₂ atmosphere. LCMS showed the reaction was complete and desired MS observed. The four parallel reactions were combined and the mixture was poured into saturated aq. NH₄Cl (10 mL) at 0° C., and then extracted with EtOAc (10 mL×3). The combined organic layers were washed with brine (15 mL×2), dried over Na₂SO₄, filtered, and concentrated under reduced pressure to give a residue. The residue was purified by prep-HPLC (column: Phenomenex Luna 80*30 mm*3 um; mobile phase: [water (0.04% HCl)-ACN]; B %: 15%-45%, 8 min) to give crude product (100 mg, crude). The crude product was further purified by prep-HPLC (column: Phenomenex Luna 80*30 mm*3 um; mobile phase: [water (0.04% HCl)-MEOH]; B %: 15%-20%, 8 min) to give (trans)-3-[[2-[(1-hydroxy-3-methyl-3H-2,1-benzoxaborol-5-yl)amino]-5-methyl-pyrimidin-4-yl]amino]tetrahydropyran-4-carbonitrile (36.7 mg) mixture of isomers as a white solid. ¹H NMR (DMSO-d₆, 400 MHz) δ 10.45 (s, 1H), 9.09 (s, 1H), 8.29 (s, 1H), 7.86 (s, 1H), 7.76-7.65 (m, 2H), 7.39 (d, J=8.8 Hz, 1H), 5.25-5.15 (m, 1H), 4.50-4.40 (m, 1H), 3.95-3.85 (m, 2H), 3.30-3.20 (m, 3H), 2.25-2.10 (m, 1H), 2.06 (s, 3H), 1.85-1.70 (m, 1H), 1.45-1.40 (m, 3H). MS (ESI): mass calcd. For C₁₉H₂₂BN₅O₃ 379.18, m/z found 380.2 [M+H]⁺. HPLC: 99.96% (220 nm), 99.97% (254 nm).

### 29. Preparation of (trans)-3-[[2-[(7 fluoro-1-hydroxy-3H-2,1-benzoxaborol-5-yl)amino]-5-methyl pyrimidin-4-yl]amino]tetrahydropyran-4-carbonitrile

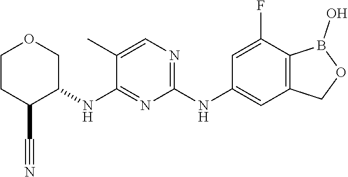 [SMILES: Cc1cnc(Nc2cc(F)c3c(c2)COB3O)nc1N[C@H]1COCC[C@@H]1C#N]

### 29.1 Preparation of (5-amino-2-bromo-3 fluoro-phenyl)methanol

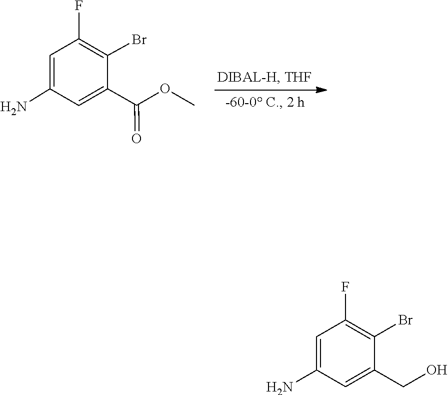

To a solution of methyl 5-amino-2-bromo-3-fluoro-benzoate (5.7 g, 23.0 mmol, 1 eq) in THF (60 mL) was added DIBAL-H (1 M in THF, 57.5 mL, 2.5 eq) drop-wise at −60° C. The mixture was stirred at −60° C. for 0.5 h. After the addition, the reaction was allowed to warm to 0° C. and stirred at 0° C. for 1.5 h. TLC showed the reaction was complete. The reaction mixture was quenched by addition aq. seignette salt (100 mL) at 0° C., and then extracted with EtOAc (100 mL×3). The combined organic layers were washed with brine (200 mL×2), dried over Na₂SO₄, filtered, and concentrated under reduced pressure to give a residue. The residue was triturated with Petroleum ether at 25° C. for 30 min to give (5-amino-2-bromo-3-fluoro-phenyl)methanol (5 g, 98% yield) as a brown solid. ¹H NMR (DMSO-d₆, 400 MHz) δ 6.63 (s, 1 H), 6.34 (dd, J=2.8, 7.6 Hz, 1H), 5.57 (s, 2H), 5.33 (t, J=6.0 Hz, 1H), 4.38 (d, J=6.0 Hz, 2H).

### 29.2 Preparation of (trans)-3-[[2-[4-bromo-3 fluoro-5-(hydroxymethyl)anilino]-5-methyl-pyrimidin-4-yl]amino]tetrahydropyran-4-carbonitrile

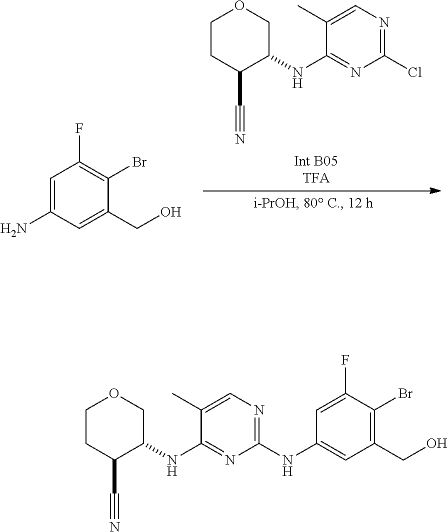

To a mixture of (5-amino-2-bromo-3-fluoro-phenyl)methanol (2.5 g, 11.3 mmol, 1 eq) and (trans)-3-[(2-chloro-5-methyl-pyrimidin-4-yl)amino]tetrahydropyran-4-carbonitrile (2.87 g, 11.36 mmol, 1 eq) in i-PrOH (30 mL) was added TFA (1.94 g, 17.0 mmol, 1.26 mL, 1.5 eq) at 25° C. The mixture was stirred at 80° C. for 12 h under N₂ atmosphere. TLC showed the reaction was complete. After cooling the reaction to room temperature, large amount of solid formed. The reaction mixture was filtered and the filter cake was washed respectively with EtOAc (20 mL), saturated aq. NaHCO₃ (20 mL) and MeCN (20 mL) to give (trans)-3-[[2-[4-bromo-3-fluoro-5-(hydroxymethyl)anilino]-5-methyl-pyrimidin-4-yl]amino]tetrahydr-opyran-4-carbonitrile (3.7 g, 74% yield) as a white solid. ¹H NMR (DMSO-d₆, 400 MHz) δ 9.40 (s, 1H), 7.90 (dd, J=2.4, 12.4 Hz, 1H), 7.80 (s, 1H), 7.68 (s, 1H), 6.76 (d, J=8.8 Hz, 1H), 5.44 (s, 1H), 4.56-4.40 (m, 3H), 3.92-3.82 (m, 2H), 3.32-3.19 (m, 2H), 3.18-3.11 (m, 1H), 2.20-2.12 (m, 1H), 1.95 (s, 3H), 1.92-1.80 (m, 1H).

### 29.3 Preparation of (trans)-3-[[2-[4-bromo-3-[[tert-butyl(dimethyl)silyl]oxymethyl]-5-fluoro-anilino]-5-methyl-pyrimidin-4-yl]amino]tetrahydropyran-4-carbonitrile

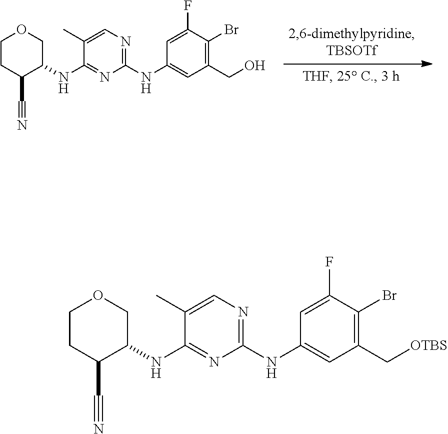

To a solution of (trans)-3-[[2-[4-bromo-3-fluoro-5-(hydroxymethyl)anilino]-5-methyl-pyrimidin-4-yl]amino]tetrahydropyran-4-carbonitrile (2.1 g, 4.81 mmol, 1 eq) and 2,6-dimethylpyridine (1.55 g, 14.4 mmol, 1.68 mL, 3 eq) in THE (30 mL) was added [tert-butyl(dimethyl)silyl] trifluoromethanesulfonate (1.91 g, 7.22 mmol, 1.5 eq) at 25° C. The mixture was stirred at 25° C. for 3 h. TLC showed the reaction was complete. The reaction mixture was quenched by addition H₂O (30 mL) at 0° C. and extracted with EtOAc (30 mL×3). The combined organic layers were washed with brine (20 mL×2), dried over Na₂SO₄, filtered, and concentrated under reduced pressure to give a residue. The residue was triturated with petroleum ether (40 mL) to give (trans)-3-[[2-[4-bromo-3-[[tert-butyl(dimethyl) silyl]oxymethyl]-5-fluoro-anilino]-5-methyl-pyrimidin-4-yl]amino]tetrahydropyran-4-carbonitrile (2.54 g, 95% yield) as a white solid. ¹H NMR (DMSO-d₆, 400 MHz) δ 9.52 (s, 1H), 7.93 (dd, J=2.0, 12.0 Hz, 1H), 7.78 (s, 1H), 7.64 (s, 1H), 6.84 (d, J=8.4 Hz, 1H), 4.67 (s, 2H), 4.54-4.42 (m, 1H), 3.92-3.83 (m, 2H), 3.27-3.10 (m, 3H), 2.22-2.14 (m, 1H), 1.96 (s, 3H), 1.89-1.75 (m, 1H), 0.94 (s, 9H), 0.13 (s, 6H).

### 29.4 Preparation of [2-[[tert-butyl(dimethyl)silyl]oxymethyl]-4-[[4-[((trans)-4-cyanotetrahydropyran-3-yl)amino]-5-methyl-pyrimidin-2-yl]amino]-6 fluoro-phenyl]boronic acid

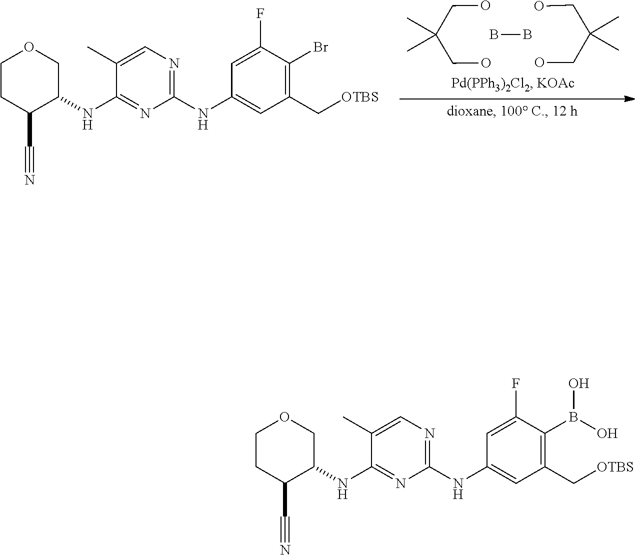

Five reactions were carried out in parallel. For each a mixture of (trans)3-[[2-[4-bromo-3-[[tert-butyl(dimethyl) silyl]oxymethyl]-5-fluoro-anilino]-5-methyl-pyrimidin-4-yl]amino]tetrahydropyran-4-carbonitrile (0.5 g, 908 umol, 1 eq), 2-(5,5-dimethyl-1,3,2-dioxaborinan-2-yl)-5,5-dimethyl-1,3,2-dioxaborinane (1.23 g, 5.45 mmol, 6 eq), Pd(PPh₃)₂Cl₂ (95.6 mg, 136 umol, 0.15 eq) and KOAc (196 mg, 2.00 mmol, 2.2 eq) in dioxane (10 mL) was stirred at 100° C. for 12 h under N₂ atmosphere. TLC showed the reaction was complete. The five parallel reactions were combined, filtered, and concentrated under reduced pressure to give a residue. The residue was triturated with MTBE (20 mL) and filtered. The filtrate was concentrated under reduced pressure to give a residue. The residue was purified by column chromatography (SiO₂, Petroleum ether/ethyl acetate=1/1 to 0/1) to give a crude product. The crude product was further purified by prep-HPLC (column: Waters Xbridge BEH C18 100*30 mm*10 um; mobile phase: [water (NH₄HCO₃)-ACN]; B %: 35%-65%, 10 min) to give [2-[[tert-butyl(dimethyl)silyl]oxymethyl]-4-[[4-[((trans)-4-cyanotetrahydropyran-3-yl)amino]-5-methyl-pyrimidin-2-yl]amino]-6-fluoro-phenyl] boronic acid (2 g) as a white solid. ¹H NMR (DMSO-d₆, 400 MHz) δ 9.18 (s, 1H), 7.99 (s, 2H), 7.77 (s, 1H), 7.52-7.47 (m, 2H), 6.69 (d, J=8.8 Hz, 1H), 4.68 (s, 2H), 4.53-4.43 (m, 1H), 3.92-3.83 (m, 2H), 3.28-3.10 (m, 3H), 2.22-2.15 (m, 1H), 1.95 (s, 3H), 1.88-1.75 (m, 1H), 0.92 (s, 9H), 0.09 (s, 6H).

### 29.5 Preparation of (trans)-3-[[2-[(7 fluoro-1-hydroxy-3H-2,1-benzoxaborol-5-yl)amino]-5-methyl pyrimidin-4-yl]amino]tetrahydropyran-4-carbonitrile

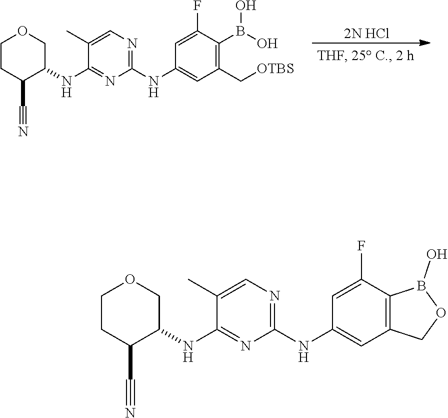 [SMILES: Cc1cnc(Nc2cc(F)c(B(O)O)c(CO[Si](C)(C)C(C)(C)C)c2)nc1N[C@H]1COCC[C@@H]1C#N ; "2N HCl" ; Cc1cnc(Nc2cc(F)c3c(c2)COB3O)nc1N[C@H]1COCC[C@@H]1C#N]

To a solution of [2-[[tert-butyl(dimethyl)silyl]oxymethyl]-4-[[4-[((trans)-4-cyanotetrahydropyran-3-yl)amino]-5-methyl-pyrimidin-2-yl]amino]-6-fluoro-phenyl]boronic acid (2 g, 3.87 mmol, 1 eq) in THF (20 mL) was added aq. HCl (2 M, 19.4 mL, 10 eq) at 25° C. The mixture was stirred at 25° C. for 2 h. LCMS showed the reaction was complete and desired MS observed. The reaction mixture was filtered and the filter cake was triturated with MTBE (20 mL) to give (trans)-3-((2-((7-fluoro-1-hydroxy-1,3-dihydrobenzo[c][1,2]oxaborol-5-yl)amino)-5-methylpyrimidin-4-yl)amino)tetrahydro-2H-pyran-4-carbonitrile (1.8 g), which was separated by SFC (column: DAICEL CHIRALPAK AD (250 mm×50 mm, 10 um); mobile phase: [0.1% NH₃H₂O ETOH]; B %: 40%-40%, 6 min) to give (trans)-3-[[2-[(7-fluoro-1-hydroxy-3H-2,1-benzoxaborol-5-yl)amino]-5-methyl-pyrimidin-4-yl]amino] tetrahydropyran-4-carbonitrile (367.9 mg, 98.91% purity, 97.08% ee, first peak, Rt=1.393 min) stereoisomer one as a white solid. ¹H NMR (DMSO-d₆, 400 MHz) δ 9.44 (s, 1H), 8.97 (s, 1H), 7.82 (s, 1H), 7.68 (s, 1H), 7.47 (d, J=11.6 Hz, 1H), 6.80 (d, J=8.8 Hz, 1H), 4.99-4.88 (m, 2H), 4.52-4.41 (m, 1H), 3.92-3.85 (m, 2H), 3.28-3.24 (m, 1H), 3.23-3.10 (m, 2H), 2.22-2.14 (m, 1H), 1.96 (s, 3H), 1.91-1.79 (m, 1H). MS (ESI): mass calcd. For C₁₈H₁₉BFN₅O₃ 383.16, m/z found 382.0 [M−H]⁻. HPLC: 98.91% (220 nm), 98.55% (254 nm) and (trans)-3-[[2-[(7-fluoro-1-hydroxy-3H-2,1-benzoxaborol-5-yl)amino]-5-methyl-pyrimidin-4-yl]amino]tetrahydropyran-4-carbonitrile (470.2 mg, 98.93% purity, 96.96% ee, second peak, Rt=1.516 min) stereoisomer two as a white solid. ¹H NMR (DMSO-d₆, 400 MHz) δ 9.44 (s, 1H), 8.97 (s, 1H), 7.82 (s, 1H), 7.68 (s, 1H), 7.47 (d, J=10.8 Hz, 1H), 6.80 (d, J=8.4 Hz, 1H), 4.99-4.88 (m, 2H), 4.52-4.41 (m, 1H), 3.92-3.85 (m, 2H), 3.28-3.24 (m, 1H), 3.23-3.10 (m, 2H), 2.22-2.14 (m, 1H), 1.96 (s, 3H), 1.91-1.79 (m, 1H). MS (ESI): mass calcd. For C₁₈H₁₉BFN₅O₃ 383.16, m/z found 382.0 [M−H]⁻. HPLC: 98.93% (220 nm), 98.83% (254 nm).

### 30a. Preparation of (R)-7-chloro-5-((5-methyl-4-((tetrahydro-2H-pyran-3-yl)amino)pyrimidin-2-yl)benzo[c][1,2]oxaborol-1(3H)-ol

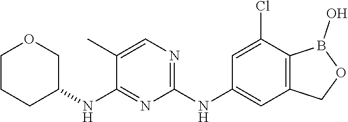 [SMILES: Cc1cnc(Nc2cc(Cl)c3c(c2)COB3O)nc1N[C@@H]1CCCOC1]

### 30a.1 Preparation of 2-chloro-5-methyl-N-[(3R)-tetrahydropyran-3-yl]pyrimidin-4-amine

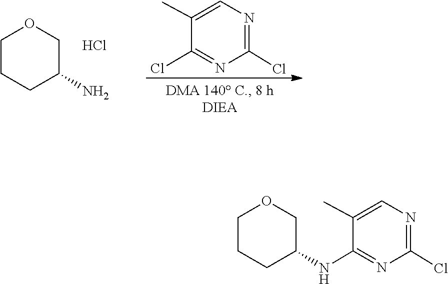

To a mixture of (3R)-tetrahydropyran-3-amine (1 g, 7.27 mmol, 1 eq, HCl salt) and 2,4-dichloro-5-methyl-pyrimidine (1.18 g, 7.27 mmol, 1 eq) in DMA (10 mL) was added DIEA (5.64 g, 43.6 mmol, 7.6 mL, 6 eq) at 20° C. The mixture was stirred at 140° C. for 8 h. TLC showed the reaction was complete. The residue was poured into H₂O (30 mL), and extracted with EtOAc (30 mL×3). The combined organic layers were washed with brine (20 mL×3), dried over Na₂SO₄ and filtered and concentrated under reduced pressure to give a residue. The residue was purified by column chromatography (SiO₂, petroleum ether/ethyl acetate=20/1 to 1/1) to give 2-chloro-5-methyl-N-[(3R)-tetrahydropyran-3-yl]pyrimidin-4-amine (1.4 g, 84.6% yield) as yellow oil. ¹H NMR (400 MHz, CDCl₃) δ 7.82 (s, 1H), 5.09 (d, J=7.2 Hz, 1H), 4.35-4.28 (m, 1H), 3.86-3.77 (m, 2H), 3.67-3.60 (m, 2H), 2.03 (s, 3H), 1.92-1.87 (m, 1H), 1.80-1.73 (m, 1H), 1.65-1.59 (m, 1H).

### 30a.2 Preparation of [2-bromo-3-chloro-5-[[5-methyl-4-[[(3R)-tetrahydropyran-3-yl]amino] pyrimidin-2-yl]amino]phenyl]methanol

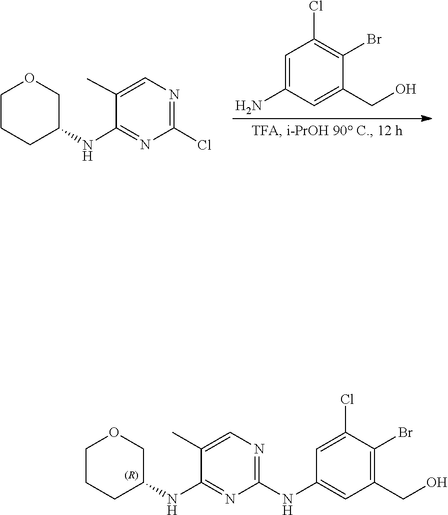

To a solution of 2-chloro-5-methyl-N-[(3R)-tetrahydropyran-3-yl]pyrimidin-4-amine (1.2 g, 5.27 mmol, 1 eq) and (5-amino-2-bromo-3-chloro-phenyl)methanol (1.25 g, 5.27 mmol, 1 eq) in i-PrOH (20 mL) was added TFA (1.20 g, 10.5 mmol, 780 uL, 2 eq) at 20° C., the mixture was stirred at 90° C. for 12 h. Solid was precipitate out. LCMS showed the reaction was complete and desired MS observed. The reaction mixture was filtered, and the filter cake was washed with aq. NaHCO₃ (20 mL) to give [2-bromo-3-chloro-5-[[5-methyl-4-[[(3R)-tetrahydropyran-3-yl]amino]pyrimidin-2-yl]amino]phenyl]methanol (1.85 g, 82.0% yield) as white solid. ¹H NMR (400 MHz, DMSO-d₆) δ 12.50-12.10 (m, 1H), 10.35 (s, 1H), 8.04 (s, 1H), 7.77 (s, 1H), 7.64 (s, 1H), 5.64-5.58 (m, 1H), 4.51 (s, 2H), 4.25-4.15 (m, 1H), 3.85-3.75 (m, 2H), 3.5-3.30 (m, 2H), 1.99 (s, 4H), 1.70-1.63 (m, 3H).

### 30a.3 Preparation of N₂-[4-bromo-3-[[tert-butyl(dimethyl)silyl]oxymethyl]-5-chloro-phenyl]-5-methyl-N₄-[(3R)-tetrahydropyran-3-yl]pyrimidine-2,4-diamine

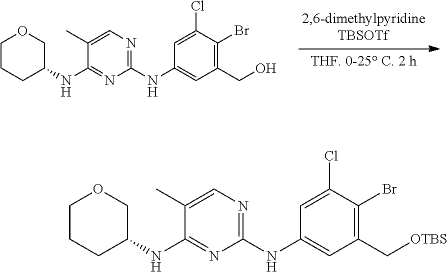 [SMILES: Cc1cnc(Nc2cc(Cl)c(Br)c(CO)c2)nc1N[C@@H]1CCCOC1 ; Cc1cnc(Nc2cc(Cl)c(Br)c(CO[Si](C)(C)C(C)(C)C)c2)nc1N[C@@H]1CCCOC1]

To a solution of [2-bromo-3-chloro-5-[[5-methyl-4-[[(3R)-tetrahydropyran-3-yl]amino]pyrimidin-2-yl]amino]phenyl]methanol (1.6 g, 3.74 mmol, 1 eq) and 2,6-dimethylpyridine (1.20 g, 11.2 mmol, 1.31 mL, 3 eq) in THE (32 mL) was added TBSOTf (1.48 g, 5.61 mmol, 1.29 mL, 1.5 eq) at 0° C. The reaction was stirred it at 25° C. for 2 h. TLC showed the reaction was complete. The reaction mixture was quenched by addition saturated aq. NH₄Cl (30 mL) at 0° C., and extracted with EtOAc (30 mL×4). The combined organic layers were dried over Na₂SO₄, filtered, and concentrated under reduced pressure to give a residue. The residue was triturated with EtOAc (15 mL) to give N2-[4-bromo-3-[[tert-butyl(dimethyl)silyl]oxymethyl]-5-chloro-phenyl]-5-methyl-N4-[(3R)-tetrahydropyran-3-yl]pyrimidine-2,4-diamine (1.8 g, 88.7% yield) as a white solid. ¹H NMR (400 MHz, DMSO-d₆) δ 9.38 (s, 1H), 8.23 (d, J=2.4 Hz, 1H), 7.76 (s, 1H), 7.70 (s, 1H), 6.31 (d, J=7.6 Hz, 1H), 4.65 (s, 2H), 4.20-4.15 (m, 1H), 3.89-3.79 (m, 2H), 3.20 (t, J=10.0 Hz, 2H), 1.93 (s, 3H), 1.71-1.61 (m, 3H), 0.94 (s, 9H), 0.13 (s, 6H).

### 30a.4 Preparation of N₂-[3-[[tert-butyl(dimethyl)silyl]oxymethyl]-5-chloro-4-(5,5-dimethyl-1,3,2-dioxaborinan-2-yl)phenyl]-5-methyl-N4-[(3R)-tetrahydropyran-3-yl]pyrimidine-2,4-diamine

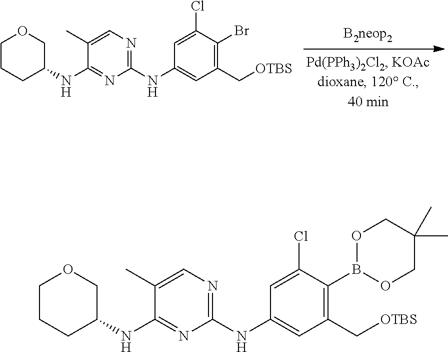 [SMILES: Cc1cnc(Nc2cc(Cl)c(Br)c(CO[Si](C)(C)C(C)(C)C)c2)nc1N[C@@H]1CCCOC1 ; Cc1cnc(Nc2cc(Cl)c(B3OCC(C)(C)CO3)c(CO[Si](C)(C)C(C)(C)C)c2)nc1N[C@@H]1CCCOC1]

Five reactions were carried out in parallel. For each a mixture of N2-[4-bromo-3-[[tert-butyl(dimethyl) silyl]oxymethyl]-5-chloro-phenyl]-5-methyl-N4-[(3R)-tetrahydropyran-3-yl]pyrimidine-2,4-diamine (450 mg, 830 umol, 1 eq), 2-(5,5-dimethyl-1,3,2-dioxaborinan-2-yl)-5,5-dimethyl-1,3,2-dioxaborinane (938 mg, 4.15 mmol, 5 eq), KOAc (244 mg, 2.49 mmol, 3 eq) and Pd(PPh₃)₂Cl₂ (58 mg, 83.0 umol, 0.1 eq) in dioxane (6 mL) was degassed and purged with N2 for 3 times. Then the mixture was heated to 120° C. and stirred at 120° C. for 40 min under N2 atmosphere. TLC showed the reaction was complete. The five parallel reactions were combined, filtered and washed with EtOAc (30 mL). The filtrate was concentrated to give a residue. The residue was purified by column chromatography (SiO2, petroleum ether/ethyl acetate=1/1 to 0/1) to give N2-[3-[[tert-butyl(dimethyl)silyl]oxymethyl]-5-chloro-4-(5,5-dimethyl-1,3,2-dioxaborinan-2-yl)phenyl]-5-methyl-N₄-[(3R)-tetrahydropyran-3-yl]pyrimidine-2,4-diamine (1.8 g, 75.3% yield) as a white solid.

### 30a.5 Preparation of (R)-7-chloro-5-((5-methyl-4-((tetrahydro-2H-pyran-3-yl)amino)pyrimidin-2-yl)benzo[c][1,2]oxaborol-1(3H)-ol

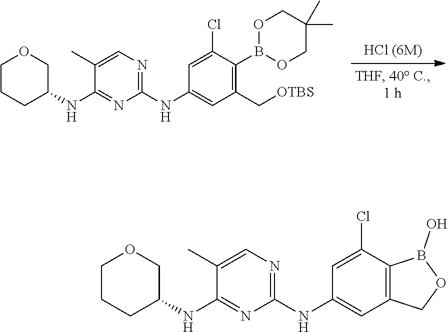 [SMILES: Cc1cnc(Nc2cc(Cl)c(B3OCC(C)(C)CO3)c(CO[Si](C)(C)C(C)(C)C)c2)nc1N[C@@H]1CCCOC1 ; Cc1cnc(Nc2cc(Cl)c3c(c2)COB3O)nc1N[C@@H]1CCCOC1]

To a solution of N2-[3-[[tert-butyl(dimethyl)silyl]oxymethyl]-5-chloro-4-(5,5-dimethyl-1,3,2-dioxaborinan-2-yl)phenyl]-5-methyl-N4-[(3R)-tetrahydropyran-3-yl]pyrimidine-2,4-diamine (2.2, 3.83 mmol, 1 eq) in THF (22 mL) was added aq. HCl (6 M, 2.55 mL, 4 eq) at 25° C., the reaction mixture was stirred at 40° C. for 1 h. Solid was precipitated out. LCMS showed the reaction was complete. The mixture was filtered. The filter cake was triturated with a mixture solvent of MeOH (3 mL) and DCM (25 mL) at 25° C. for 20 min to give (R)-7-chloro-5-((5-methyl-4-((tetrahydro-2H-pyran-3-yl)amino)pyrimidin-2-yl)benzo[c][1,2]oxaborol-1(3H)-ol (400 mg, 97.69% purity, 97.38% ee, second peak, Rt=1.284 min) as a white solid. ¹H NMR (400 MHz, DMSO-d₆) δ 11.05 (s, 1H), 9.19 (s, 1H), 8.06-8.14 (m, 1H), 7.85 (s, 1H), 7.69 (s, 1H), 7.58 (s, 1H), 4.98 (s, 2H), 4.18-4.12 (m, 1H), 3.89-3.81 (m, 2H), 3.35-3.29 (m, 2H), 2.05-1.95 (m, 4H), 1.80-1.60 (m, 3H). MS (ESI): mass calcd. For C₁₇H₂₀BClN₄O₃ 374.13, m/z found 375.1 [M+H]⁺. HPLC: 97.69% (220 nm), 99.16% (254 nm).

### 30b.1 Preparation of (S)-7-chloro-5-((5-methyl-4-((tetrahydro-2H-pyran-3-yl)amino) pyrimidin-2-yl)amino)benzo[c][1,2]oxaborol-1(3H)-ol

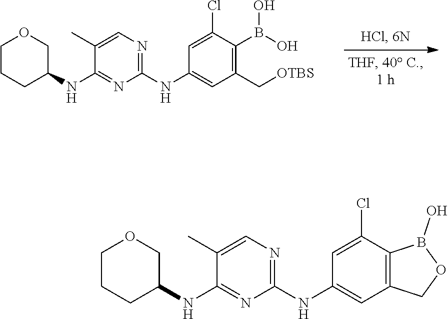 [SMILES: Cc1cnc(Nc2cc(Cl)c(B(O)O)c(CO[Si](C)(C)C(C)(C)C)c2)nc1N[C@H]1CCCOC1 ; Cc1cnc(Nc2cc(Cl)c3c(c2)COB3O)nc1N[C@H]1CCCOC1]

Derived from (3S)-tetrahydropyran-3-amine a solution of (S)-(2-(((tert-butyldimethylsilyl)oxy)methyl)-6-chloro-4-((5-methyl-4-((tetrahydro-2H-pyran-3-yl)amino)pyrimidin-2-yl)amino)phenyl)boronic acid (1 g, 1.97 mmol, 1 eq) in THF (10 mL) was added HCl (6 M, 1.32 mL, 4 eq) at 25° C. The mixture was stirred at 40° C. for 1 hr. Solid was precipitate out after 30 min reaction. TLC showed the reaction was complete. The mixture was filtered and the filter cake was washed with MeCN (20 mL) to give (S)-7-chloro-5-((5-methyl-4-((tetrahydro-2H-pyran-3-yl)amino)pyrimidin-2-yl)amino)benzo[c][1,2]oxaborol-1(3H)-ol (500 mg, 67.5 yield) as a white solid. ¹H NMR (DMSO-d₆, 400 MHz) δ 9.33 (br s, 1H), 8.83 (s, 1H), 7.82 (s, 1H), 7.77 (s, 1H), 7.73 (s, 1H), 6.33 (d, J=7.2 Hz, 1H), 4.92 (s, 2H), 4.25-4.15 (m, 1H), 3.95-3.75 (m, 2H), 3.30-3.25 (m, 1H), 3.25-3.10 (m, 1H), 2.10-1.80 (m, 4H), 1.75-1.50 (m, 3H). MS (ESI): mass calcd. For C₁₇H₂₀BClN₄O₃ 374.13, m/z found 375.1 [M+H]⁺. HPLC: 98.24% (220 nm), 99.57% (254 nm).

### 31. Preparation of (trans)-3-((2-((7-chloro-1-hydroxy-1,3-dihydrobenzo[c][1,2]oxaborol-5-yl)amino)-5-methylpyrimidin-4-yl)amino)tetrahydro-2H-pyran-4-carbonitrile

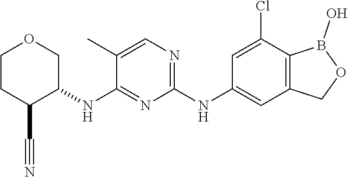 [SMILES: Cc1cnc(Nc2cc(Cl)c3c(c2)COB3O)nc1N[C@H]1COCC[C@@H]1C#N]

### 31.1 Preparation of (trans)-3-[[2-[4-bromo-3-chloro-5-(hydroxymethyl)anilino]-5-methyl-pyrimidin-4-yl]amino]tetrahydropyran-4-carbonitrile

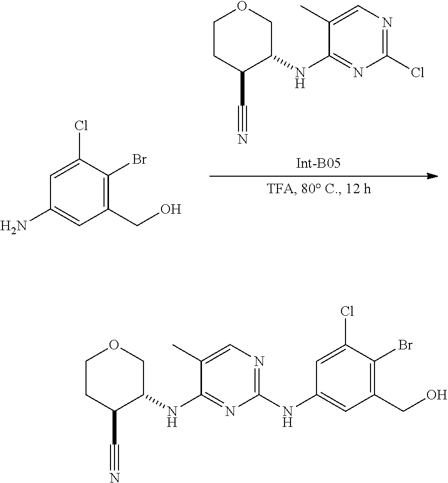 [SMILES: Nc1cc(Cl)c(Br)c(CO)c1 ; Cc1cnc(Cl)nc1N[C@H]1COCC[C@@H]1C#N ; Cc1cnc(Nc2cc(Cl)c(Br)c(CO)c2)nc1N[C@H]1COCC[C@@H]1C#N]

A mixture of (5-amino-2-bromo-3-chloro-phenyl)methanol (2.5 g, 10.5 mmol, 1 eq), (trans)-3-[(2-chloro-5-methyl-pyrimidin-4-yl)amino]tetrahydropyran-4-carbonitrile (2.67 g, 10.5 mmol, 1 eq) and TFA (1.81 g, 15.8 mmol, 1.17 mL, 1.5 eq) in i-POH (10 mL) was degassed and purged with N₂ for 3 times, and then the mixture was stirred at 80° C. for 12 h under N₂ atmosphere. TLC showed the reaction was complete. The reaction mixture was quenched by addition saturated aq. NaHCO₃ (50 mL) at 0° C. and extracted with EtOAc (50 mL×3). The combined organic layers were washed with brine (30 mL×2), dried over Na₂SO₄, filtered, and concentrated under reduced pressure to give a residue. The residue was triturated with MTBE (30 mL) at 25° C. for 10 min to give (trans)-3-[[2-[4-bromo-3-chloro-5-(hydroxymethyl)anilino]-5-methyl-pyrimidin-4-yl]amino]tetrahydropyran-4-carbonitrile (3.5 g, 73% yield) as a white solid.

### 31.2 Preparation of (trans)-3-[[2-[4-bromo-3-[[tert-butyl(dimethyl)silyl]oxymethyl]-5-chloro-anilino]-5-methyl-pyrimidin-4-yl]amino]tetrahydropyran-4-carbonitrile

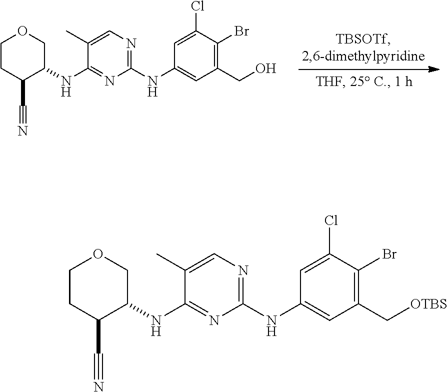 [SMILES: Cc1cnc(Nc2cc(Cl)c(Br)c(CO)c2)nc1N[C@H]1COCC[C@@H]1C#N ; Cc1cnc(Nc2cc(Cl)c(Br)c(CO[Si](C)(C)C(C)(C)C)c2)nc1N[C@H]1COCC[C@@H]1C#N]

To a solution of (trans)-3-[[2-[4-bromo-3-chloro-5-(hydroxymethyl)anilino]-5-methyl-pyrimidin-4-yl]amino]tetrahydropyran-4-carbonitrile (4.4 g, 9.72 mmol, 1 eq) in THE (50 mL) was added 2,6-dimethylpyridine (4.20 g, 38.8 mmol, 4.57 mL, 4 eq) and [tert-butyl(dimethyl)silyl] trifluoromethanesulfonate (3.85 g, 14.5 mmol, 3.35 mL, 1.5 eq). The mixture was stirred at 25° C. for 1 h. TLC showed the reaction was complete. The reaction mixture was quenched by addition saturated aq. NH₄Cl (50 mL) at 0° C. and extracted with EtOAc (50 mL×3). The combined organic layers were washed with brine (40 mL×2), dried over Na₂SO₄, filtered, and concentrated under reduced pressure to give a residue. The residue was triturated with EtOAc (30 mL) at 25° C. for 20 min to give (trans)-3-[[2-[4-bromo-3-[[tert-butyl(dimethyl)silyl]oxymethyl]-5-chloro-anilino]-5-methyl-pyrimidin-4-yl]amino]tetrahydropyran-4-carbonitrile (4.5 g, 81% yield) as a white solid.

### 31.3 Preparation of (trans)-3-((2-((3-(((tert-butyldimethylsilyl)oxy)methyl)-5-chloro-4-(5,5-dimethyl-1,3,2-dioxaborinan-2-yl)phenyl)amino)-5-methylpyrimidin-4-yl)amino) tetrahydro-2H-pyran-4-carbonitrile

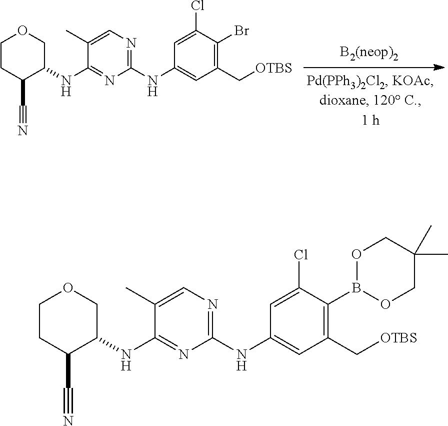 [SMILES: Cc1cnc(Nc2cc(Cl)c(Br)c(CO[Si](C)(C)C(C)(C)C)c2)nc1N[C@H]1COCC[C@@H]1C#N ; Cc1cnc(Nc2cc(Cl)c(B3OCC(C)(C)CO3)c(CO[Si](C)(C)C(C)(C)C)c2)nc1N[C@H]1COCC[C@@H]1C#N]

Nine reactions were carried out in parallel. For each a mixture of (trans)-2-[[2-[4-bromo-3-[[tert-butyl(dimethyl)silyl]oxymethyl]-5-chloro-anilino]-5-methyl-pyrimidin-4-yl]amino]cyclopentanecarbonitrile (0.5 g, 881 umol, 1 eq), 2-(5,5-dimethyl-1,3,2-dioxaborinan-2-yl)-5,5-dimethyl-1,3,2-dioxaborinane (1.02 g, 4.4 mmol, 5 eq), KOAc (267 mg, 2.72 mmol, 3 eq) and Pd(PPh₃)₂Cl₂ (63.6 mg, 88.1 umol, 0.1 eq) in dioxane (20 mL) was degassed and purged with N₂ for 3 times, and then the mixture was stirred at 120° C. for 1 h under N₂ atmosphere. TLC showed the reaction was complete. The reactions were cooled to room temperature and filtered. The nine parallel reactions were combined and the filtrate was concentrated under reduced pressure to give a residue. The residue was purified by column chromatography (SiO₂, petroleum ether/ethyl acetate=10/1 to 5/1) to give (trans)-3-((2-((3-(((tert-butyldimethylsilyl) oxy)methyl)-5-chloro-4-(5,5-dimethyl-1,3,2-dioxa borinan-2-yl)phenyl)amino)-5-methyl pyrimidin-4-yl)amino)tetrahydro-2H-pyran-4-carbonitrile (4.1 g, 84% yield) as a white solid.

### 31.4 Preparation of (trans)-3-((2-((7-chloro-1-hydroxy-1,3-dihydrobenzo[c][1,2]oxaborol-5-yl)amino)-5-methylpyrimidin-4-yl)amino)tetrahydro-2H-pyran-4-carbonitrile

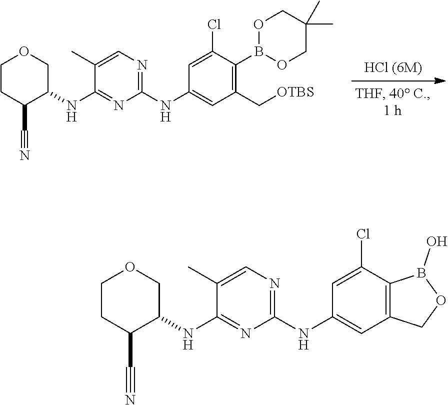 [SMILES: Cc1cnc(Nc2cc(Cl)c(B3OCC(C)(C)CO3)c(CO[Si](C)(C)C(C)(C)C)c2)nc1N[C@H]1COCC[C@@H]1C#N ; Cc1cnc(Nc2cc(Cl)c3c(c2)COB3O)nc1N[C@H]1COCC[C@@H]1C#N]

To a solution of (trans)-3-((2-((3-(((tert-butyldimethylsilyl)oxy)methyl)-5-chloro-4-(5,5-dimethyl-1,3,2-dioxaborinan-2-yl)phenyl)amino)-5-methylpyrimidin-4-yl)amino)tetrahydro-2H-pyran-4-carbonitrile (3 g, 5.00 mmol, 1 eq) in THF (30 mL) was added aq. HCl (4 M, 7.95 mL, 6 eq). The mixture was stirred at 25° C. for 1 h. LCMS showed the reaction was complete and desired MS observed. The reaction mixture was filtered and triturated with EtOAc (30 mL) to give racemic product (1 g). The racemic product (1 g) was separated by SFC (column: DAICEL CHIRALPAK AD (250 mm*30 mm, 10 um); mobile phase: [0.1% NH₃H₂O IPA]; B %: 40%-40%, 6 min) to give (trans)-3-((2-((7-chloro-1-hydroxy-1,3-dihydrobenzo[c][1,2]oxaborol-5-yl)amino)-5-methylpyrimidin-4-yl)amino)tetrahydro-2H-pyran-4-carbonitrile (313.7 mg, 98.88% purity, 100% ee, first peak, Rt=0.685 min) stereoisomer one as a white solid. ¹H NMR (DMSO-d₆, 400 MHz) δ 9.42 (s, 1H), 8.84 (s, 1H), 7.83-7.74 (m, 3H), 6.78 (d, J=8.8 Hz, 1H), 4.94 (m, 2H), 4.60-4.45 (m, 1H), 4.00-3.84 (m, 2H), 3.34-3.12 (m, 3H), 2.35-2.05 (m, 1H), 1.96 (s, 3H), 1.90-1.81 (m, 1H). MS (ESI): mass calcd. For C₁₈H₁₉BCl₂N₅O₂ 399.13, m/z found 400.1 [M+H]⁺. HPLC: 98.88% (220 nm), 99.25% (254 nm) and (trans)-3-((2-((7-chloro-1-hydroxy-1,3-dihydrobenzo[c][1,2]oxaborol-5-yl)amino)-5-methylpyrimidin-4-yl)amino)tetrahydro-2H-pyran-4-carbonitrile (333.9 mg, 98.20% purity, 98.06% ee, second peak, Rt=0.874 min) stereoisomer two as a white solid. ¹H NMR (DMSO-d₆, 400 MHz) δ 9.42 (s, 1H), 8.84 (s, 1H), 7.83-7.74 (m, 3H), 6.78 (d, J=8.8 Hz, 1H), 4.94 (m, 2H), 4.60-4.45 (m, 1H), 4.00-3.84 (m, 2H), 3.34-3.12 (m, 3H), 2.35-2.05 (m, 1H), 1.96 (s, 3H), 1.90-1.81 (m, 1H). MS (ESI): mass calcd. For C₁₈H₁₉BCl₂N₅O₂ 399.13, m/z found 400.2 [M+H]⁺. HPLC: 98.20% (220 nm), 99.33% (254 nm).

### 32. Preparation of (trans)-3-[[2-[(1-hydroxy-3,3-dimethyl-2,1-benzoxaborol-5-yl)amino]-5-methyl-pyrimidin-4-yl]amino]tetrahydropyran-4-carbonitrile

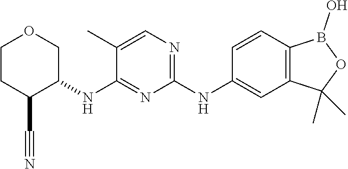 [SMILES: Cc1cnc(Nc2ccc3c(c2)C(C)(C)OB3O)nc1N[C@H]1COCC[C@@H]1C#N]

### 32.1 Preparation of (trans)-3-[[2-[4-bromo-3-(1-hydroxy-1-methyl-ethyl)anilino]-5-methyl-pyrimidin-4-yl]amino]tetrahydropyran-4-carbonitrile

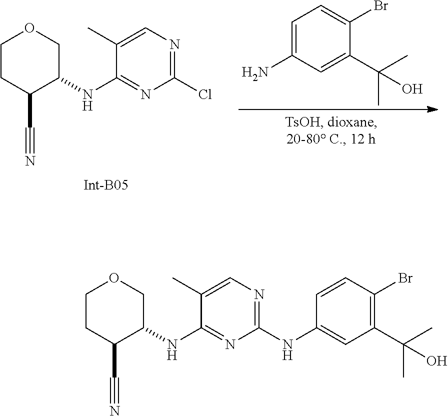 [SMILES: Cc1cnc(Cl)nc1N[C@H]1COCC[C@@H]1C#N ; CC(C)(O)c1cc(N)ccc1Br ; Cc1cnc(Nc2ccc(Br)c(C(C)(C)O)c2)nc1N[C@H]1COCC[C@@H]1C#N]

To a solution of (trans)-3-[(2-chloro-5-methyl-pyrimidin-4-yl)amino]tetrahydropyran-4-carbonitrile (600 mg, 2.37 mmol, 1 eq) in dioxane (10 mL) was added 2-(5-amino-2-bromo-phenyl) propan-2-ol (437 mg, 1.90 mmol, 0.8 eq) and TsOH·H₂O (677 mg, 3.56 mmol, 1.5 eq) at 25° C. under N₂. The mixture was heated and stirred at 80° C. for 12 h. TLC showed the reaction was complete. The reaction mixture was concentrated under reduced pressure to give a residue. The crude product was triturated with DCM (20 mL) at 25° C. to give (trans)-3-[[2-[4-bromo-3-(1-hydroxy-1-methyl-ethyl)anilino]-5-methyl-pyrimidin-4-yl]amino]tetrahydropyran-4-carbonitrile (400 mg, 37.7% yield) as an off-white solid. ¹H NMR (DMSO-d₆, 400 MHz) δ 9.08 (s, 1H), 8.13 (d, J=2.8 Hz, 1H), 7.75 (s, 1H), 7.61 (dd, J=8.4 Hz, 2.8 Hz, 1H), 7.36 (d, J=8.4 Hz, 1H), 6.64 (d, J=8.4 Hz, 1H), 5.08 (s, 1H), 4.53-4.44 (m, 1H), 3.87-3.83 (m, 2H), 3.30-3.20 (m, 2H), 3.12 (t, J=10.8 Hz, 1H), 2.16-2.12 (m, 1H), 1.94 (s, 3H), 1.61 (d, J=2.8 Hz, 6H).

### 32.2 Preparation of (trans)-3-[[2-[(1-hydroxy-3,3-dimethyl-2,1-benzoxaborol-5-yl)amino]-5-methyl-pyrimidin-4-yl]amino]tetrahydropyran-4-carbonitrile

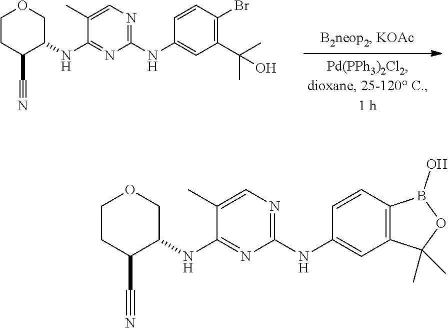 [SMILES: Cc1cnc(Nc2ccc(Br)c(C(C)(C)O)c2)nc1N[C@H]1COCC[C@@H]1C#N ; Cc1cnc(Nc2ccc3c(c2)C(C)(C)OB3O)nc1N[C@H]1COCC[C@@H]1C#N]

Four reactions were carried out in parallel. For each a solution of (trans)-3-[[2-[4-bromo-3-(1-hydroxy-1-methyl-ethyl)anilino]-5-methyl-pyrimidin-4-yl] amino]tetrahydropyran-4-carbonitrile (100 mg, 224 umol, 1 eq) and 2-(5,5-dimethyl-1,3,2-dioxaborinan-2-yl)-5,5-dimethyl-1,3,2-dioxaborinane (127 mg, 560 umol, 2.5 eq) in dioxane (3 mL) was added KOAc (44.0 mg, 448 umol, 2 eq), Pd(PPh₃)₂Cl₂ (15.7 mg, 22.4 umol, 0.1 eq) at 25° C. under N₂. The mixture was heated and stirred at 120° C. for 1 h. TLC showed the reaction was complete. The four parallel reactions were combined and filtered The reaction filtrate was concentrated under reduced pressure to give a residue. The residue was purified by prep-HPLC (column: Phenomenex Luna 80*30 mm*3 um; mobile phase: [water (0.04% HCl)-MEOH]; B %: 10%-30%, 8 min) to give (trans)-3-[[2-[(1-hydroxy-3,3-dimethyl-2,1-benzoxaborol-5-yl)amino]-5-methyl-pyrimidin-4-yl]amino]tetrahydropyran-4-carbonitrile (59 mg, 94.03% purity) as an off-white solid. ¹H NMR (DMSO-d₆, 400 MHz) δ 10.74 (s, 1H), 8.52 (d, J=8.4 Hz, 1H), 7.91 (s, 1H), 7.76 (s, 1H), 7.68 (d, J=8.0 Hz, 1H), 7.32 (dd, J=8.0 Hz, 1.6 Hz, 1H), 4.49-4.40 (m, 1H), 3.93-3.86 (m, 2H), 3.32-3.23 (m, 3H), 2.24-2.20 (m, 1H), 2.06 (d, J=7.2 Hz, 3H), 1.82-1.71 (m, 1H), 1.48 (d, J=4.8 Hz, 6H). MS (ESI): mass calcd. For C₂₀H₂₄BN₅O₃ 393.20, m/z found 394.3 [M+H]⁺. HPLC: 94.03% (220 nm), 96.18% (254 nm).

### 33. Preparation of (trans)-3-[[2-[[1-hydroxy-7-(trifluoromethyl)-3H-2,1-benzoxaborol-5-yl]amino]-5-methyl-pyrimidin-4-yl]amino]tetrahydropyran-4-carbonitrile

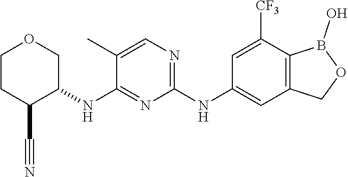 [SMILES: Cc1cnc(Nc2cc3c(c(C(F)(F)F)c2)B(O)OC3)nc1N[C@H]1COCC[C@@H]1C#N]

### 33.1 Preparation of methyl 2-amino-3-(trifluoromethyl)benzoate

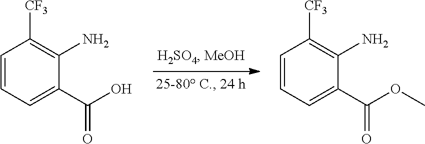

A mixture of 2-amino-3-(trifluoromethyl)benzoic acid (5 g, 24.4 mmol, 1 eq) in MeOH (100 mL) was added H₂SO₄ (7.36 g, 75.1 mmol, 4.0 mL, 3.1 eq) at 25° C. under N₂, and then the mixture was stirred 80° C. for 24 h. TLC showed the reaction was complete. The reaction mixture was concentrated under reduced pressure to give a residue. The residue was added saturated aq. NaHCO₃ (200 mL) and extracted with EtOAc (150 mL×3). The combined organic layers were washed with brine (300 mL), dried over Na₂SO₄, filtered, and concentrated under reduced pressure to give methyl 2-amino-3-(trifluoromethyl)benzoate (4 g, 75% yield) as brown oil. ¹H NMR (CDCl₃, 400 MHz) δ 8.06 (d, J=8.0 Hz, 1H), 7.59 (d, J=7.6 Hz, 1H), 6.67 (t, J=8.0 Hz, 1H), 6.47 (s, 2H), 3.89 (s, 3H).

### 33.2 Preparation of methyl 2-bromo-3-(trifluoromethyl)benzoate

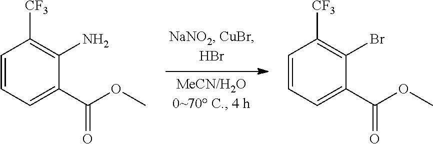

To a stirred solution of methyl 2-amino-3-(trifluoromethyl)benzoate (5 g, 22.8 mmol, 1 eq) in MeCN (30 mL) was added HBr (50.0 g, 247 mmol, 33.6 mL, 40% purity, 10.9 eq) drop-wise over 10 min at 0° C. Then a solution of NaNO₂ (1.73 g, 25.1 mmol, 1.1 eq) in H₂O (7.5 mL) was added drop-wise to the reaction over 1 h at 0° C. After addition, the solution was stirred at 0° C. for 20 min, then added CuBr (3.76 g, 26.2 mmol, 1.15 eq) in portions over 30 min at 0° C. The mixture was heated and stirred at 70° C. for 2 h. TLC showed the reaction was complete. The reaction mixture was quenched with H₂O (100 mL) at 0° C., and extracted with EtOAc (100 mL×3). The combined organic layers were washed with brine (100 mL×2), dried over Na₂SO₄, filtered, and concentrated under reduced pressure to give a residue. The residue was purified by flash silica gel chromatography (ISCO®; 40 g SepaFlash® Silica Flash Column, Eluent of 0˜10% Ethyl acetate/petroleum ether gradient @ 80 mL/min) to give methyl 2-bromo-3-(trifluoromethyl)benzoate (5 g, 77% yield) as yellow oil. ¹H NMR (CDCl₃, 400 MHz) δ 7.80 (d, J=8.0 Hz, 1H), 7.74 (d, J=7.6 Hz, 1H), 7.48 (t, J=8.0 Hz, 1H), 3.97 (s, 3H).

### 33.3 Preparation of methyl 2-bromo-5-nitro-3-(trifluoromethyl)benzoate

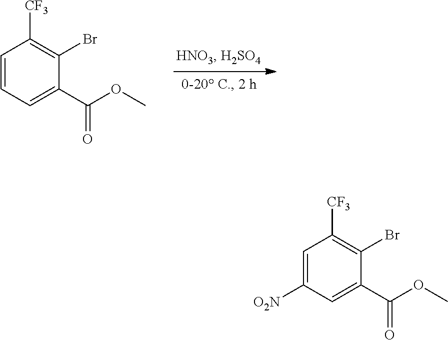 [SMILES: COC(=O)c1cccc(C(F)(F)F)c1Br ; COC(=O)c1cc([N+](=O)[O-])cc(C(F)(F)F)c1Br]

To a solution of methyl 2-bromo-3-(trifluoromethyl)benzoate (13 g, 45.9 mmol, 1 eq) in H₂SO₄ (110 mL) was drop-wise added fuming nitric acid (10.8 g, 163 mmol, 7.75 mL, 95% purity, 3.56 eq) at 0° C. After the addition, the reaction was allowed to warm to 20° C. and stirred at 20° C. for 2 h under N₂ atmosphere. TLC showed the reaction was complete. The reaction mixture was poured into ice-water (80 mL) at 0° C., and large amount of precipitate formed. The mixture was filtered and the filter cake was washed with H₂O (50 mL×3) to give methyl 2-bromo-5-nitro-3-(trifluoromethyl)benzoate (10 g, 66% yield) as a white solid. ¹H NMR (CDCl₃, 400 MHz) δ 8.65-8.60 (m, 2H), 4.04 (s, 3H).

### 33.4 Preparation of methyl 5-amino-2-bromo-3-(trifluoromethyl)benzoate

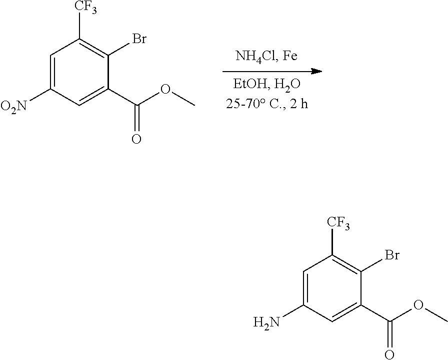 [SMILES: COC(=O)c1cc([N+](=O)[O-])cc(C(F)(F)F)c1Br ; COC(=O)c1cc(N)cc(C(F)(F)F)c1Br]

A mixture of methyl 2-bromo-5-nitro-3-(trifluoromethyl)benzoate (5 g, 15.2 mmol, 1 eq) in EtOH (60 mL) and H₂O (20 mL) was added Fe (2.55 g, 45.7 mmol, 3.00 eq) and NH₄Cl (2.45 g, 45.8 mmol, 3.00 eq) at 25° C., and then the mixture was stirred at 70° C. for 2 h. TLC showed the reaction was complete. The reaction mixture was filtered and concentrated under reduced pressure to give a residue. The residue was added H₂O (50 mL) and extracted with EtOAc (50 mL×3). The combined organic layers were washed with brine (50 mL×2), dried over anhydrous Na₂SO₄, filtered, and concentrated under reduced pressure to give methyl 5-amino-2-bromo-3-(trifluoromethyl)benzoate (4 g, 88% yield) as a brown solid. ¹H NMR (CDCl₃, 400 MHz) δ 7.08 (d, J=2.8 Hz, 1H), 7.00 (d, J=2.8 Hz, 1H), 4.13-3.96 (m, 2H), 3.95 (s, 3H).

### 33.5 Preparation of [5-amino-2-bromo-3-(trifluoromethyl)phenyl]methanol

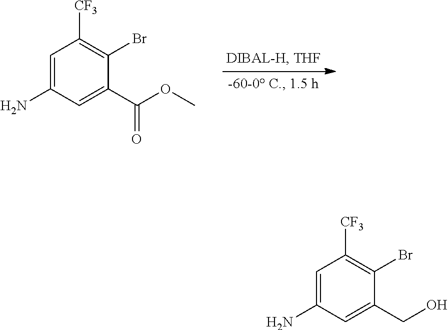 [SMILES: COC(=O)c1cc(N)cc(C(F)(F)F)c1Br ; Nc1cc(CO)c(Br)c(C(F)(F)F)c1]

To a solution of methyl 5-amino-2-bromo-3-(trifluoromethyl)benzoate (6 g, 20.1 mmol, 1 eq) in THF (60 mL) was added DIBAL-H (1 M in THF, 50.3 mL, 2.5 eq) drop-wise at −60° C. The mixture was stirred at −60° C. for 0.5 h. Then the reaction was allowed to warm to 0° C. and stirred at 0° C. for 1 h. TLC showed the reaction was complete. The reaction mixture was quenched by addition aq. seignette salt (100 mL) at 0° C., and then extracted with EtOAc (30 mL×3). The combined organic layers were washed with brine (100 mL×2), dried over Na₂SO₄, filtered, and concentrated under reduced pressure to give a residue. The residue was triturated with Petroleum ether at 25° C. for 30 min to give [5-amino-2-bromo-3-(trifluoromethyl)phenyl]methanol (5.2 g, 95% yield) as a yellow solid. ¹H NMR (CDCl₃, 400 MHz) δ 7.01 (d, J=2.8 Hz, 1H), 6.34 (d, J=2.8 Hz, 1H), 4.74 (d, J=5.6 Hz, 2H), 3.90 (s, 2H).

### 33.6 Preparation of (trans)-3-[[2-[4-bromo-3-(hydroxymethyl)-5-(trifluoromethyl)anilino]-5-methyl-pyrimidin-4-yl]amino]tetrahydropyran-4-carbonitrile

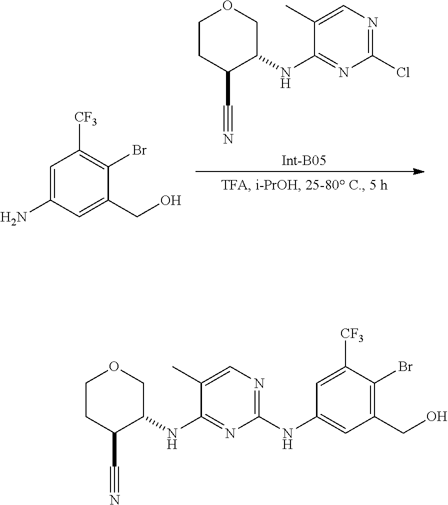

To a mixture of [5-amino-2-bromo-3-(trifluoromethyl)phenyl]methanol (3 g, 11.1 mmol, 1 eq) and (trans)-3-[(2-chloro-5-methyl-pyrimidin-4-yl)amino]tetrahydropyran-4-carbonitrile (2.81 g, 11.1 mmol, 1 eq) in i-PrOH (30 mL) was added TFA (1.90 g, 16.7 mmol, 1.23 mL, 1.5 eq) in one portion at 25° C. The mixture was stirred at 25° C. for 10 min, then heated to 80° C. and stirred for 5 hours. TLC showed the reaction was complete. The reaction mixture was filtered, and the filter cake was triturated with saturated aq. NaHCO₃ (50 mL) to give 3-[[2-[4-bromo-3-(hydroxymethyl)-5-(trifluoromethyl)anilino]-5-methyl-pyrimidin-4-yl]amino]tetrahydropyran-4-carbonitrile (5.3 g, 98% yield) as a white solid. ¹H NMR (DMSO-d₆, 400 MHz) δ 9.78 (s, 1H), 8.31 (d, J=2.4 Hz, 1H), 8.10 (s, 1H), 7.83 (s, 1H), 7.09 (s, 1H), 5.57 (s, 1H), 4.55 (s, 2H), 4.51-4.48 (m, 1H), 3.86-3.81 (m, 2H), 3.25-3.12 (m, 3H), 2.15 (d, J=10.0 Hz, 1H), 1.98 (s, 3H), 1.87-1.82 (m, 1H).

### 33.7 Preparation of (trans)-3-[[2-[4-bromo-3-[[tert-butyl(dimethyl)silyl]oxymethyl]-5-(trifluoromethyl)anilino]-5-methyl pyrimidin-4-yl]amino]tetrahydropyran-4-carbonitrile

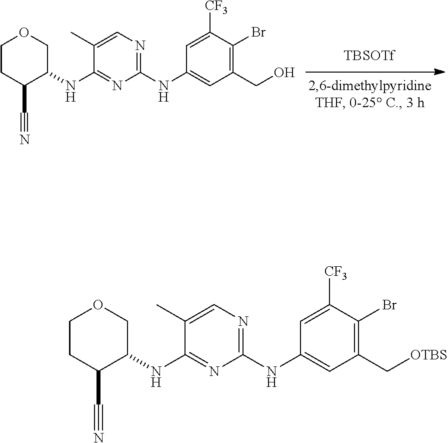

To a solution of (trans)-3-[[2-[4-bromo-3-(hydroxymethyl)-5-(trifluoromethyl)anilino]-5-methyl-pyrimidin-4-yl]amino]tetrahydropyran-4-carbonitrile (4 g, 8.2 mmol, 1 eq) and 2,6-dimethylpyridine (2.64 g, 24.7 mmol, 2.9 mL, 3 eq) in THE (60 mL) was added [tert-butyl(dimethyl)silyl] trifluoromethanesulfonate (3.26 g, 12.3 mmol, 2.8 mL, 1.5 eq) at 0° C. After the addition, the reaction was allowed to warm to 25° C. and stirred for 3 h. TLC showed the reaction was complete. The reaction mixture was quenched by addition saturated aq. NH₄Cl (100 mL) at 0° C., and then extracted with EtOAc (100 mL×3). The combined organic layers were washed with brine (100 mL×2), dried over Na₂SO₄, filtered, and concentrated under reduced pressure to give a residue. The residue was triturated with Petroleum ether (50 mL) to give (trans)-3-[[2-[4-bromo-3-[[tert-butyl(dimethyl)silyl]oxymethyl]-5-(trifluoromethyl)anilino]-5-methyl-pyrimidin-4-yl]amino]tetrahydropyran-4-carbonitrile (4.5 g, 91% yield) as a white solid. ¹H NMR (DMSO-d₆, 400 MHz) δ 9.62 (s, 1H), 8.40 (d, J=2.8 Hz, 1H), 8.12 (s, 1H), 7.80 (d, J=10.8 Hz, 1H), 6.79 (d, J=8.8 Hz, 1H), 4.73 (s, 2H), 4.51-4.48 (m, 1H), 3.86-3.83 (m, 2H), 3.28-3.24 (m, 2H), 3.23-3.12 (m, 1H), 2.17 (d, J=9.6 Hz, 1H), 1.97-1.96 (m, 3H), 1.81-1.79 (m, 1H), 0.95 (s, 9H), 0.14 (s, 6H).

### 33.8 Preparation of (trans)-3-[[2-[3-[[tert-butyl(dimethyl)silyl]oxymethyl]-4-(5,5-dimethyl-1,3,2-dioxaborinan-2-yl)-5-(trifluoromethyl)anilino]-5-methyl-pyrimidin-4-yl]amino]tetrahydropyran-4-carbonitrile

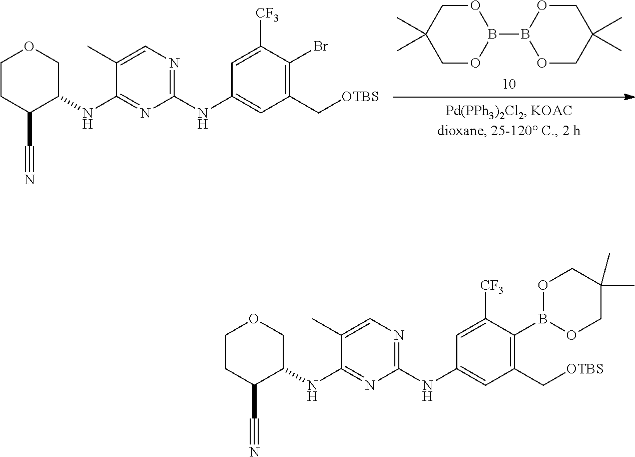

To a mixture of (trans)-3-[[2-[4-bromo-3-[[tert-butyl(dimethyl)silyl]oxymethyl]-5-(trifluoromethyl) anilino]-5-methyl-pyrimidin-4-yl]amino]tetrahydropyran-4-carbonitrile (700 mg, 1.2 mmol, 1 eq) and 2-(5,5-dimethyl-1,3,2-dioxaborinan-2-yl)-5,5-dimethyl-1,3,2-dioxaborinane (1.84 g, 8.2 mmol, 7 eq) in dioxane (30 mL) was added KOAc (343 mg, 3.5 mmol, 3 eq) and Pd(PPh₃)₂Cl₂ (82 mg, 117 umol, 0.1 eq) at 25° C. under N₂. The mixture was stirred at 120° C. for 2 h. LCMS showed the reaction was complete and desired MS observed. 6 parallel reactions were combined for work up. The reaction mixture was filtered and the filtrate concentrated in vacuum to give a residue. The residue was purified by prep-HPLC (column: C18 (250×50 mm×10 um); mobile phase: [water (NH₄HCO₃)-ACN]; B %: 75%-90%, 10 min) to give (trans)-3-[[2-[3-[[tert-butyl(dimethyl)silyl] oxymethyl]-4-(5,5-dimethyl-1,3,2-dioxaborinan-2-yl)-5-(trifluoromethyl)anilino]-5-methyl-pyrimidin-4-yl]amino]tetrahydropyran-4-carbonitrile (1.4 g) as an off-white solid. ¹H NMR (DMSO-d₆, 400 MHz) δ 9.39 (s, 1H), 8.17 (s, 1H), 8.12 (s, 1H), 7.90 (s, 1H), 7.79 (s, 1H), 6.70 (d, J=8.8 Hz, 1H), 4.74 (s, 2H), 4.50-4.46 (m, 1H), 3.89-3.85 (m, 2H), 3.70 (s, 4H), 3.32-3.24 (m, 2H), 3.16-3.12 (m, 1H), 2.19 (d, J=8.0 Hz, 1H), 1.96 (s, 3H), 1.87-1.83 (m, 1H), 1.03 (s, 6H), 0.90 (s, 9H), 0.07 (s, 6H).

### 33.9 Preparation of (trans)-3-[[2-[[1-hydroxy-7-(trifluoromethyl)-3H-2,1-benzoxaborol-5-yl] amino]-5-methyl pyrimidin-4-yl]amino]tetrahydropyran-4-carbonitrile

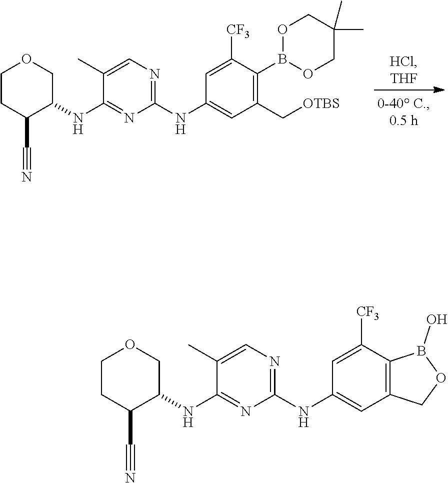

To a mixture of (trans)-3-[[2-[3-[[tert-butyl(dimethyl)silyl]oxymethyl]-4-(5,5-dimethyl-1,3,2-dioxaborinan-2-yl)-5-(trifluoromethyl)anilino]-5-methyl-pyrimidin-4-yl]amino]tetrahydropyran-4-carbonitrile (700 mg, 1.1 mmol, 1 eq) in THF (8 mL) was added HCl (6 M, 0.9 mL, 5 eq) drop-wise at 0° C. The mixture was stirred at 40° C. for 0.5 h. LCMS showed the reaction was complete and desired MS observed. 2 parallel reactions were combined for work up. The reaction mixture was filtered and the filter cake was wished with H₂O (10 mL). The crude product was triturated with MeCN (10 mL) at 25° C. for 30 min to give (trans)-3-[[2 [[1-hydroxy-7-(trifluoromethyl)-3H-2,1-benzoxaborol-5-yl]amino]-5-methyl-pyrimidin-4-yl]amino]tetrahydropyran-4-carbonitrile (800 mg, 97.53% purity) as a white solid. This material was separated by prep-SFC (column: ChiralPak IH, 250×30 mm, 10 um; mobile phase: [0.1% NH₃H₂O IPA]; B %: 25%-25%, 9 min) to give (trans)-3-[[2-[[1-hydroxy-7-(trifluoromethyl)-3H-2,1-benzoxaborol-5-yl]amino]-5-methyl-pyrimidin-4-yl]amino]tetrahydropyran-4-carbonitrile (345.4 mg, 98.39% purity, 95.36% ee, first peak, Rt=1.824 min) stereoisomer one as a white solid ¹H NMR (DMSO-d₆, 400 MHz) δ 9.74 (s, 1H), 8.93 (s, 1H), 8.06 (s, 2H), 7.84 (s, 1H), 7.05 (broad doublet, 1H), 5.06-5.00 (m, 2H), 5.49-4.44 (m, 1H), 3.90-3.85 (m, 2H), 3.27-3.21 (m, 2H), 3.17-3.11 (m, 1H), 2.19 (d, J=10.0 Hz, 1H), 1.98 (s, 3H), 1.86-1.83 (m, 1H). MS (ESI): mass calcd. For C₁₉H₁₉BF₃N₅O₃ 433.15, m/z found 434.2 [M+H]⁺. HPLC: 98.39% (220 nm), 97.98% (254 nm) and (trans)-3-[[2-[[1-hydroxy-7-(trifluoromethyl)-3H-2,1-benzoxaborol-5-yl] amino]-5-methyl-pyrimidin-4-yl]amino]tetrahydropyran-4-carbonitrile (285.4 mg, 98.34% purity, 97.14% ee, second peak, Rt=1.963 min) stereoisomer two as a white solid. ¹H NMR (DMSO-d₆, 400 MHz) δ 9.64 (s, 1H), 8.90 (s, 1H), 8.09 (d, J=5.2 Hz, 2H), 7.83 (s, 1H), 6.88 (d, J=7.6 Hz, 1H), 5.06-4.96 (m, 2H), 4.48-4.44 (m, 1H), 3.90-3.85 (m, 2H), 3.30-3.22 (m, 2H), 3.17-3.10 (m, 1H), 2.19 (d, J=10.0 Hz, 1H), 1.97 (s, 3H), 1.87-1.83 (m, 1H). MS (ESI): mass calcd. For C₁₉H₁₉BF₃N₅O₃ 433.15, m/z found 434.2 [M+H]⁺. HPLC: 98.34% (220 nm), 99.50% (254 nm).

### 34. Preparation of (trans)-3-[[2-[(2-hydroxy-1,2-benzoxaborinin-7-yl)amino]-5-methyl-pyrimidin-4-yl]amino]tetrahydropyran-4-carbonitrile

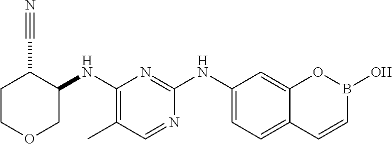 [SMILES: Cc1cnc(Nc2ccc3c(c2)OB(O)C=C3)nc1N[C@H]1COCC[C@@H]1C#N]

### 34.1 Preparation of (trans)-3-[[2-[(2,4-dimethoxyphenyl)methylamino]-5-methyl-pyrimidin-4-yl]amino]tetrahydropyran-4-carbonitrile

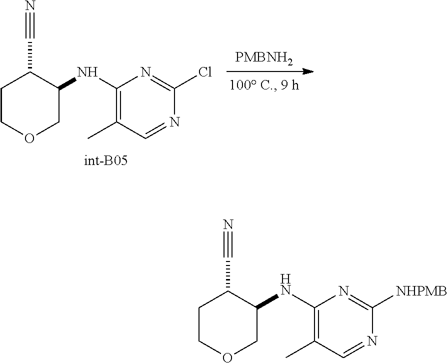

Three reactions were carried out in parallel. For each a mixture of (trans)-3-[(2-chloro-5-methyl-pyrimidin-4-yl)amino]tetrahydropyran-4-carbonitrile (650 mg, 2.57 mmol, 1 eq) and (2,4-dimethoxyphenyl)methanamine (6.45 g, 38.5 mmol, 5.81 mL, 15 eq) was stirred at 100° C. for 9 h. TLC showed the reaction was complete. The three parallel reactions were combined and the mixture was adjusted pH=4-5 with aq. HCl (2N), and added H₂O (20 mL). The aqueous layer was extracted with EtOAc (20 mL×2). The aqueous layers were adjusted pH=8-9 with saturated aq. NaHCO₃ and extracted with EtOAc (10 mL×4). The combined organics was dried over Na₂SO₄, filtered, and concentrated under reduced pressure to give a residue. The residue was triturated with DCM (10 mL) to give (trans)-3-[[2-[(2,4-dimethoxyphenyl)methylamino]-5-methyl-pyrimidin-4-yl]amino]tetrahydropyran-4-carbonitrile (2.00 g, 67% yield) as a white solid. ¹H NMR (DMSO-d₆, 400 MHz) δ 7.53 (s, 1H), 7.06 (d, J=8.0 Hz, 1H), 6.54-6.50 (m, 2H), 6.42-6.39 (m, 2H), 4.35-4.33 (m, 1H), 4.32-4.26 (m, 2H), 3.84-3.82 (m, 2H), 3.81 (s, 3H), 3.71 (s, 3H), 3.25-3.21 (m, 2H), 3.20-3.18 (m, 1H), 2.08-2.05 (m, 1H), 1.84 (s, 3H), 1.81-1.77 (m, 1H).

### 34.2 Preparation of (trans)-3-[(2-amino-5-methyl-pyrimidin-4-yl)amino] tetrahydropyran-4-carbonitrile

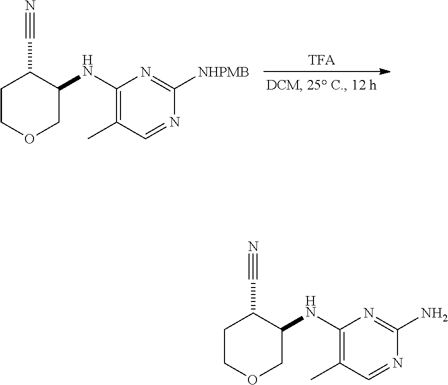

To a mixture of (trans)-3-[[2-[(2,4-dimethoxyphenyl)methylamino]-5-methyl-pyrimidin-4-yl]amino]tetrahydropyran-4-carbonitrile (1.80 g, 4.69 mmol, 1 eq) in DCM (20 mL) was added TFA (5.35 g, 46.9 mmol, 3.48 mL, 10 eq) in one portion at 25° C. under N₂. The mixture was stirred at 25° C. for 12 h. TLC showed the reaction was complete. The mixture was adjusted pH=8 with saturated aq. NaHCO₃ and diluted with H₂O (20 mL). The aqueous layer was extracted with EtOAc (20 mL×4). The combined organic layers were washed with brine (20 mL×2), dried over Na₂SO₄, filtered, and concentrated under reduced pressure to give (trans)-3-[(2-amino-5-methyl-pyrimidin-4-yl)amino]tetrahydropyran-4-carbonitrile (750 mg, 68% yield) as a white solid, which used directly into next step without further purification. ¹H NMR (DMSO-d₆, 400 MHz) δ 7.54 (s, 1H), 6.54 (d, J=8.8 Hz, 1H), 6.06 (s, 2H), 4.40-4.36 (m, 1H), 3.81-3.77 (m, 2H), 3.25-3.22 (m, 2H), 3.08-3.05 (m, 1H), 2.12-2.11 (m, 1H), 1.86 (s, 3H), 1.83-1.79 (m, 1H).

### 34.3 Preparation of 5-bromo-2-[2-(4,4,5,5-tetramethyl-1,3,2-dioxaborolan-2-yl) vinyl]phenol

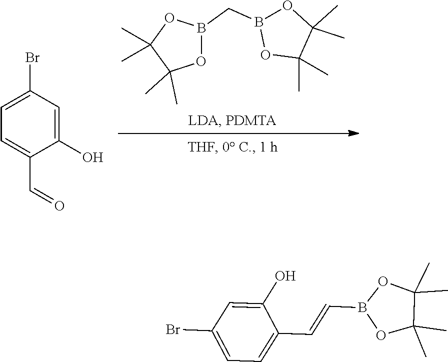

To a mixture of LDA (2 M in THF, 37.3 mL, 3 eq) in THF (50 mL) was added 4,4,5,5-tetramethyl-2-[(4,4,5,5-tetramethyl-1,3,2-dioxaborolan-2-yl)methyl]-1,3,2-dioxaborolane (20.0 g, 74.6 mmol, 3 eq) and N-[2-(dimethylamino)ethyl]-N,N,N′-trimethyl-ethane-1,2-diamine (8.62 g, 49.7 mmol, 10.4 mL, 2 eq) drop-wise at 0° C. under N₂. The mixture was stirred at 0° C. for 30 min. The reaction mixture was added 4-bromo-2-hydroxy-benzaldehyde (5 g, 24.8 mmol, 1 eq) and stirred for 30 min at 0° C. TLC showed the reaction was complete. The mixture was quenched with saturated aq. NH₄Cl (100 mL) and extracted with EtOAc (1000 mL×3). The combined organic layers were washed by brine (50 mL×2), dried over Na₂SO₄, filtered, and concentrated under reduced pressure to give a residue. The residue was purified by flash silica gel chromatography (ISCO®; 80 g SepaFlash® Silica Flash Column, Eluent of 10˜15% Ethyl acetate/petroleum ether gradient @ 100 mL/min) to give 5-bromo-2-[2-(4,4,5,5-tetramethyl-1,3,2-dioxaborolan-2-yl)vinyl]phenol (5 g, 61% yield) as a yellow oil. ¹H NMR (CDCl₃, 400 MHz) δ 7.56 (d, J=18.4 Hz, 1H), 7.33 (d, J=8.4 Hz, 1H), 7.05 (d, J=8.4 Hz, 1H), 7.00 (s, 1H), 6.16 (d, J=18.4 Hz, 1H), 5.49 (br d, 1H), 1.29 (s, 12H).

### 34.4 Preparation of 7-bromo-2-hydroxy-1,2-benzoxaborinine

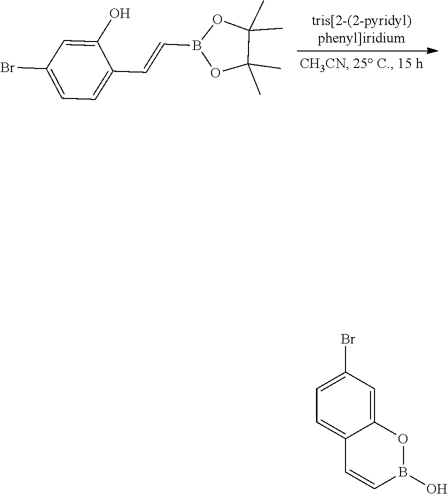 [SMILES: CC1(C)OB(/C=C/c2ccc(Br)cc2O)OC1(C)C ; OB1C=Cc2ccc(Br)cc2O1]

Three reactions were carried out in parallel. For each a mixture of 5-bromo-2-[2-(4,4,5,5-tetramethyl-1,3,2-dioxaborolan-2-yl)vinyl]phenol (500 mg, 1.54 mmol, 1 eq) in MeCN (11 mL) was added tris[2-(2-pyridyl)phenyl]iridium (10 mg, 15 umol, 0.01 eq) in one portion at 25° C. under N₂. The reaction was stirred and irradiated using 34W blue LED lamps for 15 h. TLC showed the reaction was complete. The three parallel reactions were combined, filtered, and concentrated under reduced pressure to give a residue. The residue was purified by prep-HPLC (column: Waters Xbridge Prep OBD C18 150*40 mm*10 um; mobile phase: [water (10 mM NH₄HCO₃)-ACN]; B %: 30%-60%, 8 min) to obtain 7-bromo-2-hydroxy-1,2-benzoxaborinine (240 mg, 23% yield) as a white solid. ¹H NMR (DMSO-d₆, 400 MHz) δ 9.17 (s, 1H), 7.78 (d, J=12.0 Hz, 1H), 7.47 (d, J=8.0 Hz, 1H), 7.45 (d, J=2 Hz, 1H), 7.33 (dd, J=8, 2 Hz, 1H), 6.17 (d, J=12.0 Hz, 1H).

### 34.5 Preparation of (trans)-3-[[2-[(2-hydroxy-1,2-benzoxaborinin-7-yl)amino]-5-methyl-pyrimidin-4-yl]amino]tetrahydropyran-4-carbonitrile

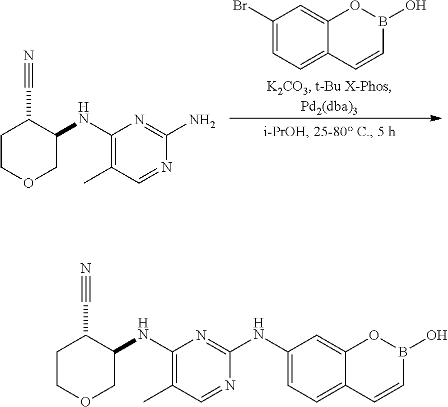

Five reactions were carried out in parallel. For each a mixture of (trans)-3-[(2-amino-5-methyl-pyrimidin-4-yl)amino]tetrahydropyran-4-carbonitrile (150 mg, 643 umol, 1 eq) and 7-bromo-2-hydroxy-1,2-benzoxaborinine (231 mg, 1.03 mmol, 1.6 eq) in i-PrOH (3 mL) was added K₂CO₃ (160 mg, 1.16 mmol, 1.8 eq), Pd₂(dba)₃ (82.4 mg, 90 umol, 0.14 eq) and t-Bu Xphos (191 mg, 450 umol, 0.7 eq) in one portion at 25° C. under N₂. The mixture was stirred at 80° C. for 5 h. LCMS showed the reaction was complete and desired MS observed. The five parallel reactions were combined and the mixture was adjusted pH=6 with aq. HCl (2N) and concentrated under reduced pressure to give a residue. The residue was purified by prep-HPLC (column: Phenomenex Luna 80*30 mm*3 um; mobile phase: [water (HCl)-ACN]; B %: 10%-30%, 8 min) to give (trans)-3-[[2-[(2-hydroxy-1,2-benzoxaborinin-7-yl)amino]-5-methyl-pyrimidin-4-yl]amino]tetrahydropyran-4-carbonitrile (383 mg, 31% yield, 99.45% purity) as a yellow solid. ¹H NMR (DMSO-d₆, 400 MHz) δ 10.40 (s, 1H), 9.00 (s, 1H), 8.26 (s, 1H), 7.81 (s, 1H), 7.78 (s, 1H), 7.52 (d, J=8.4 Hz, 1H), 7.40 (s, 1H), 7.35 (d, J=8.4 Hz, 1H), 6.06 (d, J=11.6 Hz, 1H), 4.49-4.43 (m, 1H), 3.88-3.83 (m, 2H), 3.29-3.26 (m, 3H), 2.20-2.17 (m, 1H), 2.04 (s, 3H), 1.88-1.85 (m, 1H). MS (ESI): mass calcd. For C₁₉H₂₀BN₅O₃ 377.17, m/z found 378.2 [M+H]⁺.

HPLC: 99.45% (220 nm), 99.18% (254 nm).

### 35. Preparation of 3-[[2-[(2-hydroxy-1,2-benzoxaborinin-6-yl)amino]-5-methyl-pyrimidin-4-yl]amino]tetrahydropyran-4-carbonitrile

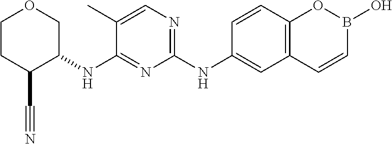 [SMILES: Cc1cnc(Nc2ccc3c(c2)C=CB(O)O3)nc1N[C@H]1COCC[C@@H]1C#N]

### 35.1 Preparation of 6-bromo-2-hydroxy-1,2-benzoxaborinine

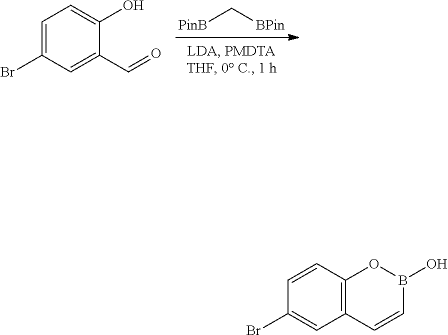

To a mixture of LDA (2 M, 18.6 mL, 2.5 eq) in THF (50 mL) was added 4,4,5,5-tetramethyl-2-[(4,4,5,5-tetramethyl-1,3,2-dioxaborolan-2-yl)methyl]-1,3,2-dioxaborolane (10.0 g, 37.3 mmol, 2.5 eq) and N′-[2-(dimethylamino)ethyl]-N,N,N′-trimethyl-ethane-1,2-diamine (7.76 g, 44.7 mmol, 9.35 mL, 3 eq) drop-wise at 0° C. under N₂. The mixture was stirred at 0° C. for 30 min. To the resulting mixture was added a solution of 5-bromo-2-hydroxy-benzaldehyde (3 g, 14.9 mmol, 1 eq) in THF (10 mL) drop-wise at 0° C. The mixture was stirred for 30 min at 0° C. TLC showed the reaction was complete. The mixture was quenched with saturated aq. NH₄Cl (30 mL) and extracted with EtOAc (30 mL×2). The combined organic layers were washed with brine (30 mL×2), dried over Na₂SO₄, filtered, and concentrated under reduced pressure to give a residue. The residue was purified by flash silica gel chromatography (ISCO®; 40 g SepaFlash® Silica Flash Column, Eluent of 20˜40% Ethyl acetate/petroleum ether gradient @ 75 mL/min) to get 6-bromo-2-hydroxy-1,2-benzoxaborinine (500 mg, 14% yield) as a white solid. ¹H NMR (CDCl₃, 400 MHz) δ 7.69 (d, J=12 Hz, 1H), 7.55 (d, J=2.4 Hz, 1H), 7.46 (dd, J=8.8, 2.4 Hz, 1H), 7.14 (d, J=8.8 Hz, 1H), 6.28 (d, J=12 Hz, 1H).

### 35.2 Preparation of tert-butyl N-(2-hydroxy-1,2-benzoxaborinin-6-yl)carbamate

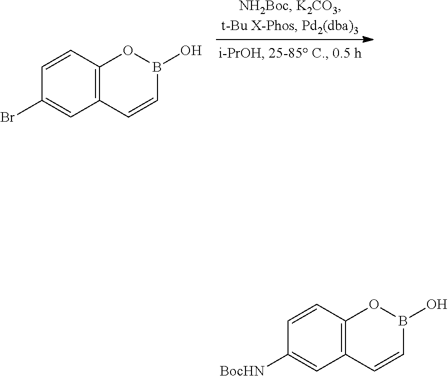 [SMILES: "NH2Boc, K2CO3,t-Bu X-Phos, Pd2(dba)3" ; OB1C=Cc2cc(Br)ccc2O1 ; CC(C)(C)OC(=O)Nc1ccc2c(c1)C=CB(O)O2]

Three reactions were carried out in parallel. For each a mixture of 6-bromo-2-hydroxy-1,2-benzoxaborinine (1 g, 4.45 mmol, 1 eq) and tert-butyl carbamate (625 mg, 5.34 mmol, 1.2 eq) in i-PrOH (30 mL) was added K₂CO₃ (1.54 g, 11.1 mmol, 2.5 eq), t-Bu Xphos (188 mg, 444 umol, 0.1 eq) and Pd₂(dba)₃ (81.5 mg, 89 umol, 0.02 eq) in one portion at 25° C. under N₂. The mixture was heated and stirred at 85° C. for 0.5 h. TLC showed the reaction was complete. The three parallel reactions were combined and quenched with H₂O (20 mL) and extracted with EtOAc (30 mL×2). The combined organic layers were washed with brine (20 mL×2), dried over Na₂SO₄, filtered, and concentrated under reduced pressure to give a residue. The residue was purified by flash silica gel chromatography (ISCO®; 80 g SepaFlash® Silica Flash Column, Eluent of 20˜30% Ethyl acetate/petroleum ether gradient @ 100 mL/min) to give a crude product. The crude product was further purified by prep-HPLC (column: Waters Xbridge BEH C18 100*30 mm*10 um; mobile phase: [water (NH₄HCO₃)-ACN]; B %: 30%-50%, 10 min) to give tert-butyl N-(2-hydroxy-1,2-benzoxaborinin-6-yl)carbamate (1 g, 28% yield) as a white solid. ¹H NMR (CDCl₃, 400 MHz) δ 7.73 (d, J=12 Hz, 1H), 7.58 (s, 1H), 7.23 (dd, J=8.8, 2.8 Hz, 1H), 7.16 (d, J=8.8 Hz, 1H), 6.22 (d, J=12 Hz, 1H), 1.54 (s, 9H).

### 35.3 Preparation of 2-hydroxy-1,2-benzoxaborinin-6-amine hydrochloride

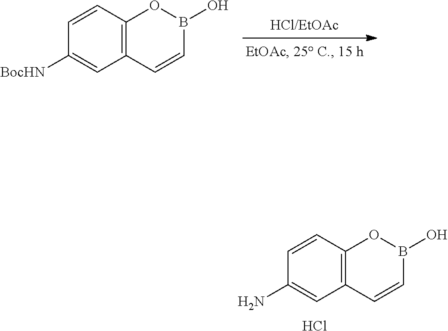 [SMILES: CC(C)(C)OC(=O)Nc1ccc2c(c1)C=CB(O)O2 ; Nc1ccc2c(c1)C=CB(O)O2 ; Cl]

To a mixture of tert-butyl N-(2-hydroxy-1,2-benzoxaborinin-6-yl)carbamate (1 g, 3.83 mmol, 1 eq) in EtOAc (5 mL) was added HCl/EtOAc (4 N, 38.3 mL, 80 eq) in one portion at 25° C. under N₂. The mixture was stirred at 25° C. for 15 h. TLC showed the reaction was complete. The mixture was filtered to give 2-hydroxy-1,2-benzoxaborinin-6-amine hydrochloride (601 mg, 78% yield, HCl) as an off-white solid. ¹H NMR (DMSO-d₆, 400 MHz) δ 10.03 (s, 3H), 9.12 (s, 1H), 7.83 (d, J=12 Hz, 1H), 7.46 (d, J=2.4 Hz, 1H), 7.35 (dd, J=8.4, 2.4 Hz, 1H), 7.18 (d, J=8.4 Hz, 1H), 6.22 (d, J=12 Hz, 1H).

### 35.4 Preparation of (trans)-3-[[2-[(2-hydroxy-1,2-benzoxaborinin-6-yl)amino]-5-methyl-pyrimidin-4-yl]amino]tetrahydropyran-4-carbonitrile

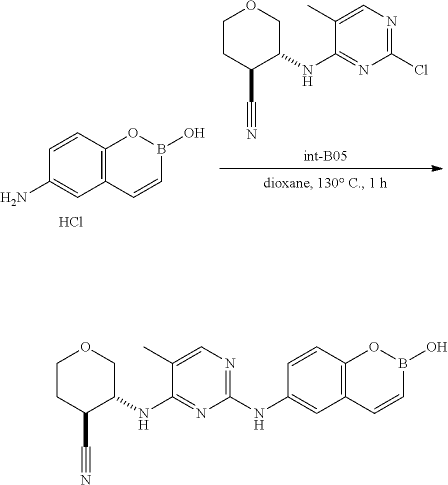 [SMILES: Nc1ccc2c(c1)C=CB(O)O2 ; Cc1cnc(Cl)nc1N[C@H]1COCC[C@@H]1C#N ; Cl ; Cc1cnc(Nc2ccc3c(c2)C=CB(O)O3)nc1N[C@H]1COCC[C@@H]1C#N]

Five reactions were carried out in parallel. For each a mixture of 2-hydroxy-1,2-benzoxaborinin-6-amine hydrochloride (100 mg, 506 umol, 1 eq, HCl) and (trans)-3-[(2-chloro-5-methyl-pyrimidin-4-yl)amino]tetrahydropyran-4-carbonitrile (141 mg, 557 umol, 1.1 eq) in dioxane (2 mL) was stirred at 130° C. for 1 h. TLC showed the reaction was complete. The five parallel reactions were combined and quenched with a mixture solvent of H₂O (0.5 mL) and THF (3 mL) and concentrated to give a residue. The residue was purified by flash silica gel chromatography (ISCO®; 2 g SepaFlash® Silica Flash Column, Eluent of 7˜10% DCM/MeOH @ 60 mL/min) to give (trans)-3-[[2-[(2-hydroxy-1,2-benzoxaborinin-6-yl)amino]-5-methyl-pyrimidin-4-yl]amino]tetrahydropyran-4-carbonitrile (535 mg, 50% yield) as a white solid. ¹H NMR (DMSO-d₆, 400 MHz) δ 12.25 (s, 1H), 10.28 (s, 1H), 9.02 (s, 1H), 8.20 (s, 1H), 7.80-7.68 (m, 3H), 7.44 (d, J=8.4 Hz, 1H), 7.24 (d, J=8.4 Hz, 1H), 6.17 (d, J=12 Hz, 1H), 4.41-4.38 (m, 1H), 3.89-3.85 (m, 2H), 3.40-3.37 (m, 1H), 3.27-3.22 (m, 2H), 2.19-2.15 (m, 1H), 2.03 (s, 3H), 1.81-1.79 (m, 1H). MS (ESI): mass calcd. For C19H20BN5O3 377.17, m/z found 378.2 [M+H]⁺. HPLC: 98.25% (220 nm), 98.43% (254 nm).

### 36. Preparation of (trans)-3-((2-((8 fluoro-2-hydroxy-2H-benzo[e][1,2] oxaborinin-6-yl)amino)-5-methylpyrimidin-4-yl)amino)tetrahydro-2H-pyran-4-carbonitrile

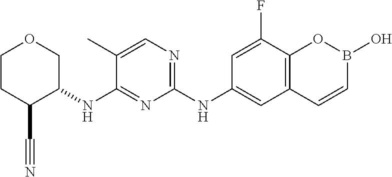 [SMILES: Cc1cnc(Nc2cc(F)c3c(c2)C=CB(O)O3)nc1N[C@H]1COCC[C@@H]1C#N]

### 36.1 Preparation of 5-bromo-3-fluoro-2-hydroxybenzaldehyde

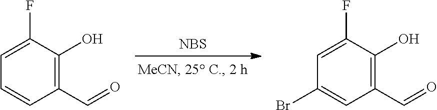 [SMILES: O=Cc1cccc(F)c1O ; "NBS" ; O=Cc1cc(Br)cc(F)c1O]

To a solution of 3-fluoro-2-hydroxy-benzaldehyde (10 g, 71.3 mmol, 1 eq) in MeCN (100 mL) was added NBS (12.8 g, 72.0 mmol, 1.01 eq) and NH₄OAc (550 mg, 7.14 mmol, 0.1 eq) at 25° C. The mixture was stirred at 25° C. for 2 hrs. LCMS showed 3-fluoro-2-hydroxy-benzaldehyde was consumed completely and one main peak with desired mass was detected. The reaction mixture was quenched by addition saturated aq. NH₄Cl (100 mL) at 0° C. and then diluted with H₂O (200 mL) and extracted with EtOAc (100 mL×3). The combined organic layers were washed with brine (200 mL), dried over Na₂SO₄, filtered, and concentrated under reduced pressure to give a residue. The residue was purified by flash silica gel chromatography (ISCO®; 120 g SepaFlash® Silica Flash Column, Eluent of 0˜25% Ethyl acetate/petroleum ether gradient @ 100 mL/min) to give compound 5-bromo-3-fluoro-2-hydroxy-benzaldehyde (14.5 g, 92% yield) as a light yellow solid. ¹H NMR (400 MHz, CHLOROFORM-d) 8=10.89 (s, 1H), 9.88 (s, 1H), 7.55-7.47 (m, 2H).

### 36.2 Preparation of (E)-4-bromo-2 fluoro-6-(2-(4,4,5,5-tetramethyl-1,3,2-dioxaborolan-2-yl)vinyl)phenol

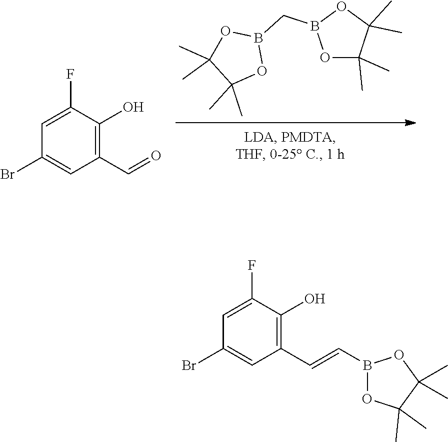

To a mixture of LDA (2 M in THF, 82.7 mL, 2.5 eq) in THF (140 mL) was drop-wise added N′-[2-(dimethylamino)ethyl]-N,N,N′-trimethyl-ethane-1,2-diamine (PMDTA, 22.9 g, 132 mmol, 27.6 mL, 2 eq) and 4,4,5,5-tetramethyl-2-[(4,4,5,5-tetramethyl-1,3,2-dioxaborolan-2-yl)methyl]-1,3,2-dioxaborolane (35.4 g, 132 mmol, 2 eq) at 0° C. After addition, the mixture was stirred at 0° C. for 20 min, and then a solution of 5-bromo-3-fluoro-2-hydroxy-benzaldehyde (14.5 g, 66.2 mmol, 1 eq) in THF (20 mL) was drop-wise added at 0° C. After the addition, the reaction was allowed to warm to 25° C. and stirred for 40 min. LCMS showed 5-bromo-3-fluoro-2-hydroxy-benzaldehyde was consumed completely and one main peak with desired mass was detected. The reaction mixture was quenched with aq. NH₄Cl (100 mL) and extracted with EtOAc (80 mL×3). The combined organic layers were washed with brine (100 mL), dried over anhydrous Na₂SO₄, filtered, and concentrated under reduced pressure to give a residue. The residue was purified by flash silica gel chromatography (ISCO®; 120 g SepaFlash® Silica Flash Column, eluent of 0˜15% ethyl acetate/petroleum ether gradient @ 100 mL/min) to obtain compound 4-bromo-2-fluoro-6-[(E)-2-(4,4,5,5-tetramethyl-1,3,2-dioxaborolan-2-yl)vinyl]phenol (24 g, 60% purity) as light yellow oil.

### 36.3 Preparation of 6-bromo-8 fluoro-2-hydroxy-1,2-benzoxaborinine

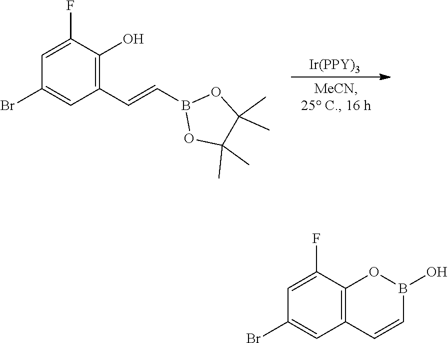 [SMILES: CC1(C)OB(/C=C/c2cc(Br)cc(F)c2O)OC1(C)C ; "Ir(PPY)3" ; OB1C=Cc2cc(Br)cc(F)c2O1]

Six reactions were carried out in parallel. For each a solution of 4-bromo-2-fluoro-6-[(E)-2-(4,4,5,5-tetramethyl-1,3,2-dioxaborolan-2-yl)vinyl] phenol (4 g, 6.98 mmol, 60% purity, 1 eq) in MeCN (30 mL) was added tris[2-(2-pyridyl)phenyl]iridium (114 mg, 175 μmol, 0.025 eq). The reaction was stirred and irradiated using 34 W blue LED lamps at 25° C. for 16 h. TLC showed the reaction was complete. The six parallel reactions were combined for work up. The reaction mixture was diluted with H₂O (100 mL) and extracted with ethyl acetate (200 mL×3). The combined organic layers were washed with brine (40 mL×2), dried over Na₂SO₄, filtered, and concentrated under reduced pressure to give a residue. The residue was purified by flash silica gel chromatography (ISCO®; 120 g SepaFlash® Silica Flash Column, eluent of 0˜25% ethyl acetate/petroleum ether gradient @ 100 mL/min) to obtain 6-bromo-8-fluoro-2-hydroxy-1,2-benzoxaborinine (10 g, 85% purity) as a yellow solid. ¹H NMR (400 MHz, DMSO-d₆) δ=9.44 (s, 1H), 7.79 (d, J=12.0 Hz, 1H), 7.68-7.60 (m, 2H), 6.26 (d, J=12.0 Hz, 1H).

### 36.4 Preparation of tert-butyl (8 fluoro-2-hydroxy-2H-benzo[e][1,2]oxaborinin-6-yl) carbamate

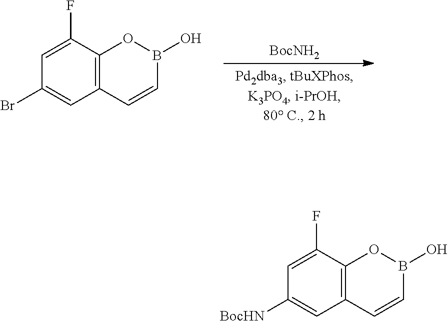 [SMILES: OB1C=Cc2cc(Br)cc(F)c2O1 ; "BocNH2" ; CC(C)(C)OC(=O)Nc1cc(F)c2c(c1)C=CB(O)O2]

A mixture of 6-bromo-8-fluoro-2-hydroxy-1,2-benzoxaborinine (10 g, 35.0 mmol, 85% purity, 1 eq), tert-butyl carbamate (4.92 g, 42.0 mmol, 1.2 eq), Pd₂(dba)₃ (3.21 g, 3.50 mmol, 0.1 eq), K₃PO₄ (11.1 g, 52.50 mmol, 1.5 eq) and ditert-butyl-[2-(2,4,6-triisopropylphenyl)phenyl]phosphane (1.49 g, 3.50 mmol, 0.1 eq) in i-PrOH (150 mL) was degassed and purged with N₂ for 3 times, and then the mixture was stirred at 80° C. for 2 h under N₂ atmosphere. LCMS showed the reaction was complete and one peak with desired mass was detected. The reaction mixture was quenched by addition H₂O (250 mL) and extracted with ethyl acetate (200 mL×3). The combined organic layers were washed with brine (200 mL), dried over Na₂SO₄, filtered, and concentrated under reduced pressure to give a residue. The residue was purified by flash silica gel chromatography (ISCO®; 40 g SepaFlash® Silica Flash Column, eluent of 0˜24% ethyl acetate/petroleum ether gradient @ 90 mL/min) to obtain tert-butyl (8-fluoro-2-hydroxy-2H-benzo[e][1,2]oxaborinin-6-yl)carbamate (1.8 g, 18.6% yield) as a gray solid. ¹H NMR (400 MHz, DMSO-d₆) δ=9.48 (s, 1H), 9.17 (s, 1H), 7.74 (d, J=12.0 Hz, 1H), 7.45-7.35 (m, 2H), 6.20 (d, J=12.0 Hz, 1H), 1.47 (s, 9H).

### 36.5 Preparation of 6-amino-8 fluoro-2H-benzo[e][1,2]oxaborinin-2-ol hydrochloride

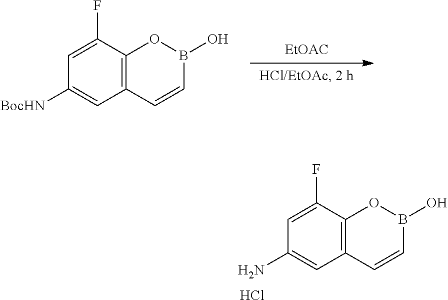 [SMILES: CC(C)(C)OC(=O)Nc1cc(F)c2c(c1)C=CB(O)O2 ; Nc1cc(F)c2c(c1)C=CB(O)O2 ; Cl]

To a solution of tert-butyl (8-fluoro-2-hydroxy-2H-benzo[e][1,2]oxaborinin-6-yl)carbamate (1.7 g, 6.09 mmol, 1 eq) in EtOAc (10 mL) was added HCl/EtOAc (4 M, 54 mL) at 25° C. The mixture was stirred at 25° C. for 2 h. TLC showed the reaction was complete. The reaction mixture was concentrated under reduced pressure to give a residue. The residue was triturated with MTBE (15 mL) at 25° C. for 10 min to obtain 6-amino-8-fluoro-2H-benzo[e][1,2]oxaborinin-2-ol hydrochloride (1.4 g, HCl salt) as a gray solid. ¹H NMR (400 MHz, DMSO-d₆) δ 7.84 (d, J=12.0 Hz, 1H), 7.41-7.23 (m, 2H), 6.28 (d, J=12.0 Hz, 1H).

### 36.6 Preparation of 3-((2-((8 fluoro-2-hydroxy-2H-benzo[e][1,2]oxaborinin-6-yl) amino)-5-methylpyrimidin-4-yl)amino)tetrahydro-2H-pyran-4-carbonitrile

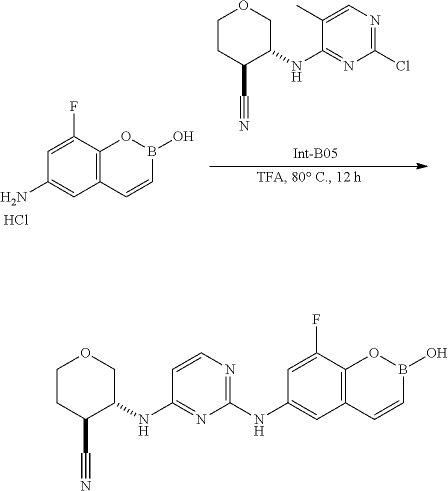

To a solution of 6-amino-8-fluoro-2H-benzo[e][1,2]oxaborinin-2-ol (1.3 g, 6.03 mmol, 1 eq, HCl) in i-PrOH (18 mL) was added (trans)-3-[(2-chloro-5-methyl-pyrimidin-4-yl)amino]tetrahydropyran-4-carbonitrile (1.52 g, 6.03 mmol, 1 eq) and TFA (1.03 g, 9.05 mmol, 1.5 eq). The mixture was stirred at 80° C. for 12 h. LCMS showed the reaction was complete and desired MS observed. The reaction mixture was quenched by addition H₂O (5 mL) and THE (4 mL), and then concentrated under reduced pressure to give a residue. The residue was triturated with MTBE (15 mL) at 25° C. for 30 min to obtain (trans)3-((2-((8-fluoro-2-hydroxy-2H-benzo[e][1,2]oxaborinin-6-yl)amino)-5-methylpyrimidin-4-yl)amino)tetrahydro-2H-pyran-4-carbonitrile (780 mg, 32% yield) as a gray solid. ¹H NMR (400 MHz, DMSO-d₆) δ 10.27 (s, 1H), 9.1 (s, 1H), 8.15 (s, 1H), 7.84-7.76 (m, 2H), 7.60-7.52 (m, 2H), 6.25 (d, J=12.0 Hz, 1H), 4.45-4.34 (m, 1H), 3.90-3.85 (m 2H), 3.28-3.21 (m, 3H), 2.23-2.15 (m, 1H), 2.03 (s, 3H), 1.84-1.71 (m, 1H). This material was separated by SFC (column: DAICEL CHIRALPAK AD (250 mm*30 mm, 10 um); mobile phase: [0.1% NH₃H₂O IPA]; B %: 30%-30%, 5 min) to obtain (trans)-3-((2-((8-fluoro-2-hydroxy-2H-benzo[e][1,2] oxaborinin-6-yl)amino)-5-methylpyrimidin-4-yl)amino)tetrahydro-2H-pyran-4-carbonitrile (195.9 mg, 98.80% purity, 97.40% ee, first peak, Rt=3.435 min) stereoisomer one as a white solid, ¹H NMR (400 MHz, DMSO-d₆) δ=9.10 (s, 2H), 7.79-7.65 (m, 4H), 6.71 (d, J=8.4 Hz, 1H), 6.17 (d, J=11.6 Hz, 1H), 4.52-4.40 (m, 1H), 3.95-3.85 (m 2H), 3.29-3.09 (m, 3H), 2.23-2.15 (m, 1H), 1.95 (s, 3H), 1.90-1.80 (m 1H). MS (ESI): mass calcd. For C₁₉H₁₉BFN₅O₃ 395.16, m/z found 396.0 [M+H]⁺. HPLC: 98.80% (220 nm), 99.37% (254 nm)′ and (trans)-3-((2-((8-fluoro-2-hydroxy-2H-benzo[e][1,2]oxaborinin-6-yl)amino)-5-methylpyrimidin-4-yl)amino)tetrahydro-2H-pyran-4-carbonitrile (299.1 mg, 98.23% purity, 95.72% ee, second peak, Rt=3.670 min) stereoisomer two as a white solid. ¹H NMR (400 MHz, DMSO-d₆) δ=9.10 (s, 2H), 7.80-7.65 (m, 4H), 6.72 (d, J=8.4 Hz, 1H), 6.17 (d, J=12.0 Hz, 1H), 4.54-4.39 (m, 1H), 3.5-3.85 (m, 2H), 3.30-3.10 (m, 3H), 2.23-2.15 (m, 1H), 1.95 (s, 3H), 1.90-1.80 (m, 1H). MS (ESI): mass calcd. For C₁₉H₁₉BFN₅O₃ 395.16, m/z found 396.1 [M+H]⁺. HPLC: 98.23% (220 nm), 99.34% (254 nm).

### 37. Preparation of (trans)-3-[[2-[[2-hydroxy-8-(trifluoromethyl)-1,2-benzoxaborinin-6-yl]amino]-5-methyl-pyrimidin-4-yl]amino]tetrahydropyran-4-carbonitrile

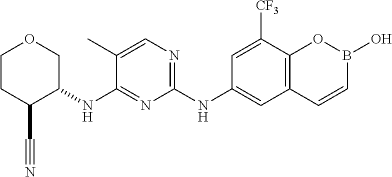 [SMILES: Cc1cnc(Nc2cc3c(c(C(F)(F)F)c2)OB(O)C=C3)nc1N[C@H]1COCC[C@@H]1C#N]

### 37.1 Preparation of 2-hydroxy-3-(trifluoromethyl)benzaldehyde

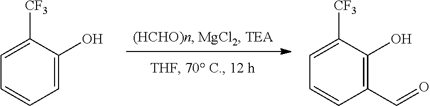 [SMILES: Oc1ccccc1C(F)(F)F ; O=Cc1cccc(C(F)(F)F)c1O]

To a solution of 2-(trifluoromethyl)phenol (14 g, 86.3 mmol, 1 eq) in THF (300 mL) was added paraformaldehyde (20.7 g, 690 mmol, 19.0 mL, 8 eq), MgCl₂ (12.3 g, 129 mmol, 1.5 eq) and TEA (30.5 g, 302 mmol, 42.1 mL, 3.5 eq) at 25° C. The mixture was heated and stirred at 70° C. for 12 h. TLC showed the reaction was complete. The reaction mixture was quenched by addition H₂O (100 mL) at 0° C. and extracted with EtOAc (50 mL×3). The combined organic layers were washed with brine (50 mL×3), dried over Na₂SO₄, filtered, and concentrated under reduced pressure to give a residue. The residue was purified by column chromatography (SiO₂, petroleum ether/ethyl acetate=20/1 to 5/1) to give 2-hydroxy-3-(trifluoromethyl)benzaldehyde (10.7 g, 65.1% yield) as yellow oil. ¹H NMR (CDCl₃, 400 MHz) δ 11.74 (s, 1H), 9.97 (s, 1H), 7.84 (d, J=8.0 Hz, 1H), 7.78 (d, J=7.6 Hz, 1H), 7.13 (t, J=7.6 Hz, 1H).

### 37.2 Preparation of 5-bromo-2-hydroxy-3-(trifluoromethyl)benzaldehyde

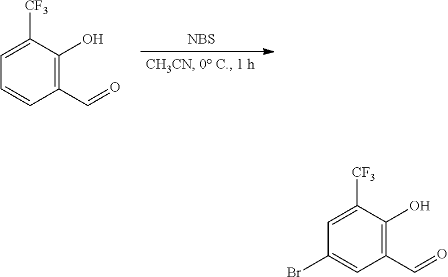 [SMILES: O=Cc1cccc(C(F)(F)F)c1O ; "NBS" ; O=Cc1cc(Br)cc(C(F)(F)F)c1O]

To a solution of 2-hydroxy-3-(trifluoromethyl)benzaldehyde (10.7 g, 56.3 mmol, 1 eq) in MeCN (150 mL) was added NBS (12.0 g, 67.5 mmol, 1.2 eq) at 0° C. The mixture was stirred at 0° C. for 1 h. TLC showed the reaction was complete. The reaction mixture was quenched by addition H₂O (200 mL) at 0° C. and extracted with EtOAc (50 mL×3). The combined organic layers were washed with saturated aq. Na₂SO₃ (60 mL) and brine (50 mL×3), dried over Na₂SO₄, filtered, and concentrated under reduced pressure to give a residue. The residue was purified by column chromatography (SiO₂, petroleum ether/ethyl acetate=1/0 to 10/1) to give 5-bromo-2-hydroxy-3-(trifluoromethyl)benzaldehyde (10 g, 66.0% yield) as brown oil. ¹H NMR (CDCl₃, 400 MHz) δ 11.63 (s, 1H), 9.91 (s, 1H), 7.93 (d, J=2.4 Hz, 1H), 7.89 (d, J=2.4 Hz, 1H).

### 37.3 Preparation of 4-bromo-2-[(E)-2-(4,4,5,5-tetramethyl-1,3,2-dioxaborolan-2-yl)vinyl]-6-(trifluoromethyl)phenol

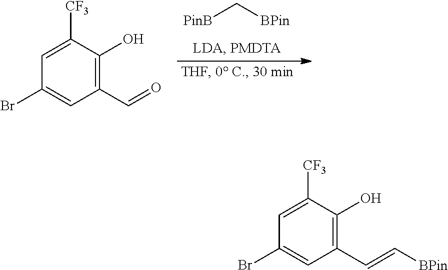

To a solution of LDA (2 M, 40.9 mL, 2.2 eq) in THF (50 mL) was drop-wise added N-[2-(dimethylamino)ethyl]-N,N,N′-trimethyl-ethane-1,2-diamine (PMDTA, 12.8 g, 74.3 mmol, 15.5 mL, 2 eq) and 4,4,5,5-tetramethyl-2-[(4,4,5,5-tetramethyl-1,3,2-dioxaborolan-2-yl)methyl]-1,3,2-dioxaborolane (19.9 g, 74.3 mmol, 2 eq) at 0° C. After addition, the mixture was stirred at 0° C. for 20 min, and then a solution of 5-bromo-2-hydroxy-3-(trifluoromethyl)benzaldehyde (10 g, 37.1 mmol, 1 eq) in THE (50 mL) was added drop-wise at 0° C. The resulting mixture was stirred at 0° C. for 10 min. The reaction mixture was quenched by addition H₂O (70 mL) and extracted with EtOAc (50 mL×3). The combined organic layers were washed with brine (50 mL×3), dried over Na₂SO₄, filtered, and concentrated under reduced pressure to give a residue. The residue was purified by column chromatography (SiO₂, petroleum ether/ethyl acetate=1/0 to 3/1) to give 4-bromo-2-[(E)-2-(4,4,5,5-tetramethyl-1,3,2-dioxaborolan-2-yl)vinyl]-6-(trifluoromethyl)phenol (11.5 g, 78.7% yield) as yellow oil.

### 37.4 Preparation of 6-bromo-2-hydroxy-8-(trifluoromethyl)-1,2-benzoxaborinine

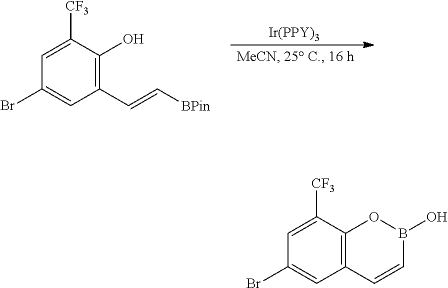

Two reactions were carried out in parallel. For each a solution of 4-bromo-2-[(E)-2-(4,4,5,5-tetramethyl-1,3,2-dioxaborolan-2-yl)vinyl]-6-(trifluoromethyl)phenol (5.65 g, 14.4 mmol, 1 eq) in MeCN (20 mL) was added tris[2-(2-pyridyl)phenyl]iridium (94 mg, 143 umol, 0.01 eq). The reaction was stirred and irradiated using 34 W blue LED lamps at 25° C. for 16 h. TLC showed the reaction was complete. The two parallel reactions were combined and the reaction mixture was filtered and concentrated under reduced pressure to give a residue. The residue was purified by column chromatography (SiO₂, petroleum ether/ethyl acetate=10/1 to 1/1) to give 6-bromo-2-hydroxy-8-(trifluoromethyl)-1,2-benzoxaborinine (4.4 g, 52.2% yield) as yellow oil. ¹H NMR (CDCl₃, 400 MHz) δ 7.75 (s, 1H), 7.71 (s, 1H), 7.68 (d, J=12.0 Hz, 1H), 6.40 (d, J=12.0 Hz, 1H).

### 37.5 Preparation of tert-butyl N-[2-hydroxy-8-(trifluoromethyl)-1,2-benzoxaborinin-6-yl]carbamate

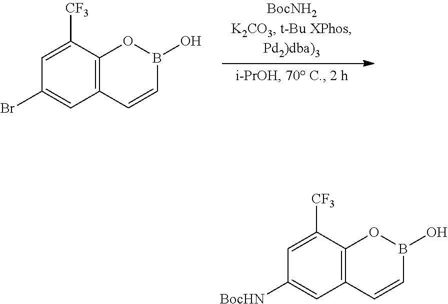 [SMILES: OB1C=Cc2cc(Br)cc(C(F)(F)F)c2O1 ; CC(C)(C)OC(=O)Nc1cc2c(c(C(F)(F)F)c1)OB(O)C=C2]

Two reactions were carried out in parallel. For each a mixture of 6-bromo-2-hydroxy-8-(trifluoromethyl)-1,2-benzoxaborinine (0.5 g, 1.71 mmol, 1 eq), tert-butyl carbamate (300 mg, 2.56 mmol, 1.5 eq), t-Bu XPhos (72 mg, 170 umol, 0.1 eq), K₂CO₃ (353 mg, 2.56 mmol, 1.5 eq) and Pd₂(dba)₃ (156 mg, 170 umol, 0.1 eq) in i-PrOH (10 mL) was degassed and purged with N₂ for 3 times, and then the mixture was heated and stirred at 70° C. for 2 h under N₂ atmosphere. TLC showed the reaction was complete. The two parallel reactions were combined for work up. The reaction mixture was filtered, quenched by addition H₂O (20 mL) at 0° C., and extracted with EtOAc (10 mL×3). The combined organic layers were washed with brine (10 mL×3), dried over Na₂SO₄, filtered, and concentrated under reduced pressure to give a residue. The residue was purified by column chromatography (SiO₂, petroleum ether/ethyl acetate=10/1) to give tert-butyl N-[2-hydroxy-8-(trifluoromethyl)-1,2-benzoxaborinin-6-yl]carbamate (0.8 g) as a brown solid.

### 37.6 Preparation of 2-hydroxy-8-(trifluoromethyl)-1,2-benzoxaborinin-6-amine (hydrogen chloride)

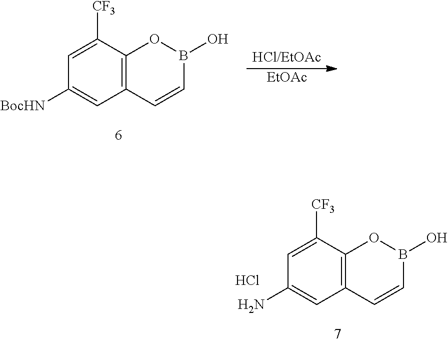 [SMILES: "EtOAc" ; CC(C)(C)OC(=O)Nc1cc2c(c(C(F)(F)F)c1)OB(O)C=C2 ; Cl ; Nc1cc2c(c(C(F)(F)F)c1)OB(O)C=C2]

To a solution of tert-butyl N-[2-hydroxy-8-(trifluoromethyl)-1,2-benzoxaborinin-6-yl]carbamate (0.8 g, 2.44 mmol, 1 eq) in EtOAc (20 mL) was added HCl/EtOAc (4 M, 6.1 mL, 10 eq). The mixture was stirred at 25° C. for 1 h. TLC showed the reaction was complete. The reaction mixture was concentrated under reduced pressure to give a residue. The residue was triturated with MTBE (10 mL) and filtered to give 2-hydroxy-8-(trifluoromethyl)-1,2-benzoxaborinin-6-amine (hydrogen chloride) (0.6 g, 92.99% yield) as a white solid. ¹H NMR (DMSO-d₆, 400 MHz) δ 9.34 (s, 1H), 7.83 (d, J=12.0 Hz, 1H), 7.50 (s, 2H), 7.22 (s, 1H), 6.92 (s, 1H), 6.27 (d, J=12.0 Hz, 1H).

### 37.7 Preparation of (trans)-3-[[2-[[2-hydroxy-8-(trifluoromethyl)-1,2-benzoxaborinin-6-yl]amino]-5-methyl-pyrimidin-4-yl]amino]tetrahydropyran-4-carbonitrile

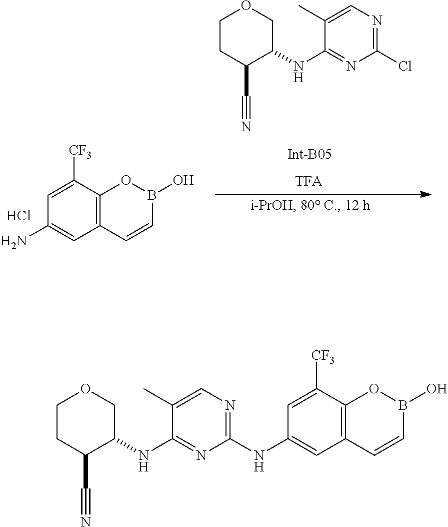

Four reactions were carried out in parallel. For each, to a solution of 2-hydroxy-8-(trifluoromethyl)-1,2-benzoxaborinin-6-amine (hydrogen chloride) (0.3 g, 1.13 mmol, 1 eq) in i-PrOH (5 mL) was added TFA (193 mg, 1.70 mmol, 1.5 eq) and (trans)-3-[(2-chloro-5-methyl-pyrimidin-4-yl)amino]tetrahydropyran-4-carbonitrile (285 mg, 1.13 mmol, 1 eq). The mixture was heated and stirred at 80° C. for 12 h. LCMS showed the reaction was complete and desired MS observed. The four parallel reactions were combined for work up. The reaction mixture was added H₂O (0.4 mL), the resulting mixture was concentrated under reduced pressure to give a crude product. The crude product was further purified by column chromatography (SiO₂, petroleum ether/ethyl acetate=1/1 to 0/1) to give (trans)-3-[[2-[[2-hydroxy-8-(trifluoromethyl)-1,2-benzoxaborinin-6-yl]amino]-5-methyl-pyrimidin-4-yl]amino]tetrahydropyran-4-carbonitrile (558.6 mg, 1.25 mmol, 27.7% yield, 97.33% purity) as an off-white solid. ¹H NMR (DMSO-d₆, 400 MHz) δ 10.37 (s, 1H), 9.37 (s, 1H), 8.20 (s, 1H), 7.88 (d, J=6.4 Hz, 2H), 7.82-7.76 (m, 2H), 6.24 (d, J=11.6 Hz, 1H), 4.35-4.25 (m, 1H), 3.83-3.75 (m, 2H), 3.20-3.12 (m, 3H), 2.12 (d, J=10.8 Hz, 1H), 1.98 (s, 3H), 1.74-1.62 (m, 1H). MS (ESI): mass calcd. For C₂₀H₁₉BF₃N₅O₃ 445.15, m/z found 444.1 [M−H]⁻. HPLC: 97.33% (220 nm), 98.72% (254 nm).

### 38. Preparation of (trans)-3-[[2-[(2-hydroxy-3-methyl-1,2-benzoxaborinin-6-yl)amino]-5-methyl-pyrimidin-4-yl]amino]tetrahydropyran-4-carbonitrile

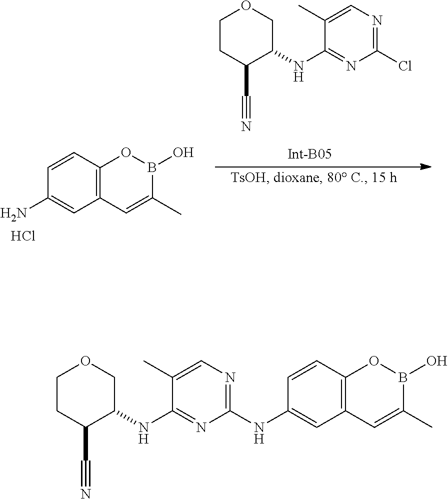

Two reactions were carried out in parallel. For each a mixture of 2-hydroxy-3-methyl-1,2-benzoxaborinin-6-amine hydrochloride (180 mg, 851 umol, 1 eq, HCl salt) and (trans)-3-[(2-chloro-5-methyl-pyrimidin-4-yl)amino]tetrahydropyran-4-carbonitrile (215 mg, 851 umol, 1 eq) in dioxane (8 mL) was added TsOH·H₂O (146 mg, 851 umol, 1 eq). The mixture was heated and stirred at 80° C. for 15 h. TLC showed the reaction was complete. The two parallel reactions were combined and concentrated under reduced pressure to give a residue. The residue was purified by prep-HPLC (column: Phenomenex Luna 80*30 mm*3 um; mobile phase: [water (0.04% HCl)-ACN]; B %: 10%-40%, 8 min) to give 3-[[2-[(2-hydroxy-3-methyl-1,2-benzoxaborinin-6-yl)amino]-5-methyl-pyrimidin-4-yl]amino]tetrahydropyran-4-carbonitrile (157.2 mg, 23% yield, 99.97% purity) as a white solid. ¹H NMR (DMSO-d₆, 400 MHz) δ 12.27 (s, 1H), 10.49 (s, 1H), 9.00 (s, 1H), 8.47 (d, J=8 Hz, 1H), 7.79 (s, 1H), 7.66 (s, 1H), 7.48 (s, 1H), 7.34 (dd, J=8.8, 2.4 Hz, 1H), 7.22 (d, J=8.8 Hz, 1H), 4.39-4.37 (m, 1H), 3.89-3.85 (m, 2H), 3.42-3.39 (m, 1H), 3.28-3.25 (m, 2H), 2.20-2.17 (m, 1H), 2.04 (s, 3H), 2.02 (s, 3H), 1.80-1.76 (m, 1H). MS (ESI): mass calcd. For C₂₀H₂₂BN₅O₃ 391.18, m/z found 392.2 [M+H]⁺. HPLC: 99.97% (220 nm), 99.98% (254 nm).

### 39. Preparation of (trans)-3-((2-((2-hydroxy-4-isopropyl-2H-benzo[e][1,2]oxaborinin-6-yl)amino)-5-methylpyrimidin-4-yl)amino)tetrahydro-2H-pyran-4-carbonitrile

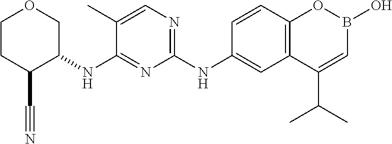 [SMILES: Cc1cnc(Nc2ccc3c(c2)C(C(C)C)=CB(O)O3)nc1N[C@H]1COCC[C@@H]1C#N]

### 39.1 Preparation of 1-(5-bromo-2-hydroxyphenyl)-2-methylpropan-1-one

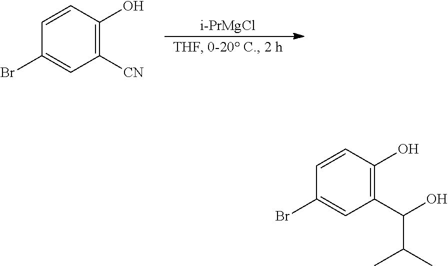

i-PrMgCl (2 M in THF, 227 mL, 3 eq) was drop-wise added to a stirred solution of 5-bromo-2-hydroxybenzonitrile (30 g, 151 mmol, 1 eq) in THF (300 mL) at 0° C. under N₂ in 30 mins. The resulting solution was allowed to warm to 20° C. and stirred for 2 h. TLC showed the reaction was complete. The solution was then cooled to 0° C., and aq. HCl (2 M, 100 mL) was added. Then the mixture was stirred and heated at 50° C. for 1 h. After cooling to room temperature, the reaction was added EtOAc (300 mL) and water (10 mL). The organic layer was washed with brine (100 mL×2), dried over Na₂SO₄, filtered, and concentrated under reduced pressure to give a residue. The residue was purified by flash column chromatography to obtain 1-(5-bromo-2-hydroxyphenyl)-2-methylpropan-1-one (35 g, 95% yield) as light yellow gum. 1H NMR (400 MHz, CDCl₃) δ 12.42 (s, 1H), 7.88 (s, 1H), 7.54 (dd, J=2.4, 8.8 Hz, 1H), 6.91 (d, J=8.8 Hz, 1H), 3.61-3.45 (m, 1H), 1.26 (d, J=6.8 Hz, 6H).

### 39.2 Preparation of 6-bromo-4-isopropyl-2H-benzo[e][1,2]oxaborinin-2-ol

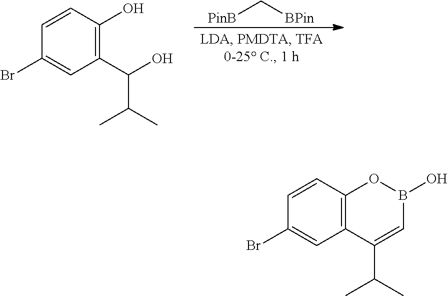

To a solution of LDA (2 M in THF, 72.0 mL, 2.5 eq) in THF (100 mL) was added N-[2-(dimethylamino)ethyl]-N,N,N′-trimethylethane-1,2-diamine (PMDTA, 20.0 g, 115 mmol, 24 mL, 2 eq) and a solution of 4,4,5,5-tetramethyl-2-[(4,4,5,5-tetramethyl-1,3,2-dioxaborolan-2-yl)methyl]-1,3,2-dioxaborolane (30.9 g, 115 mmol, 2 eq) in THF (40 mL) at 0° C. under N₂. The mixture was stirred at 0° C. for 20 min. Then drop-wise added a solution of 1-(5-bromo-2-hydroxyphenyl)-2-methylpropan-1-one (14 g, 57.5 mmol, 1 eq) in THF (2 mL) at 25° C., and then the mixture was stirred at 25° C. for 40 min. TLC showed the reaction was complete. The reaction mixture was quenched with aq. NH₄Cl (150 mL) and extracted with EtOAc (250 mL×3). The combined organic layers were washed with brine (100 mL), dried over anhydrous Na₂SO₄, filtered, and concentrated under reduced pressure to give a residue, which was purified by silica gel chromatography (petroleum ether/ethyl acetate=100:5 to 1:1) to give 6-bromo-4-isopropyl-2H-benzo[e][1,2]oxaborinin-2-ol (10 g, 65% yield) as light yellow solid.

¹H NMR (400 MHz, DMSO-d₆) δ 8.97 (s, 1H), 7.82 (s, 1H), 7.54 (d, J=8.4 Hz, 1H), 7.20 (d, J=8.4 Hz, 1H), 6.03 (s, 1H), 3.31-3.11 (m, 1H), 1.21-1.16 (m, 6H).

### 39.3 Preparation of tert-butyl (2-hydroxy-4-isopropyl-2H-benzo[e][1,2]oxaborinin-6-yl)carbamate

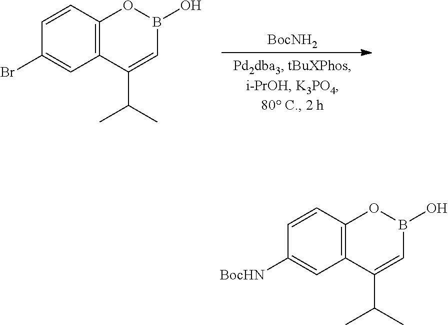

A mixture of tert-butyl carbamate (7.23 g, 61.7 mmol, 1.64 eq), K₃PO₄ (13.1 g, 61.7 mmol, 1.64 eq), Pd₂(dba)₃ (1.88 g, 2.06 mmol, 0.055 eq) and t-BuXphos (1.75 g, 4.11 mmol, 0.11 eq) and 6-bromo-4-isopropyl-2H-benzo[e][1,2]oxaborinin-2-ol (10 g, 37.5 mmol, 1 eq) in 2-propanol (50 mL) was degassed and purged with N₂ for 3 times, and then the mixture was stirred at 80° C. for 2 h. TLC showed the reaction was complete. The reaction was quenched with H₂O (100 ml) and extracted with EtOAc (250 ml×2). The combined organic phases were washed with brine (100 mL×2), dried over Na₂SO₄, filtered, and concentrated under reduced pressure to give a residue. The residue was purified by silica column (petroleum ether:EtOAc=10:1 to 1:1) to give tert-butyl (2-hydroxy-4-isopropyl-2H-benzo[e][1,2]oxaborinin-6-yl)carbamate (7.5 g, 66% yield) as a yellow solid. ¹H NMR (400 MHz, DMSO-d₆) δ=9.27 (s, 1H), 8.70 (s, 1H), 7.86 (s, 1H), 7.51-7.39 (m, 1H), 7.14-7.07 (m, 1H), 5.95 (s, 1H), 3.14-3.03 (m, 1H), 1.49-1.43 (m, 9H), 1.22-1.17 (m, 6H).

### 39.4 Preparation of 6-amino-4-isopropyl-2H-benzo[e][1,2]oxaborinin-2-ol (hydrogen chloride)

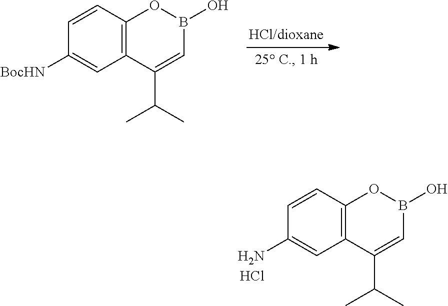 [SMILES: CC(C)C1=CB(O)Oc2ccc(NC(=O)OC(C)(C)C)cc21 ; Cl ; CC(C)C1=CB(O)Oc2ccc(N)cc21]

To a solution of tert-butyl (2-hydroxy-4-isopropyl-2H-benzo[e][1,2]oxaborinin-6-yl)carbamate (1.8 g, 5.94 mmol, 1 eq) in dioxane (10 mL) was added HCl/dioxane (4 M, 10 mL, 6.74 eq) at 25° C. for 1 h. TLC showed the reaction was complete. The reaction was concentrated to give 6-amino-4-isopropyl-2H-benzo[e][1,2]oxaborinin-2-ol (hydrogen chloride) (1.4 g, 98% yield, HCl salt) as a yellow solid, which used in next step directly with-out further purification.

### 39.5 Preparation of (trans)-3-((2-((2-hydroxy-4-isopropyl-2H-benzo[e][1,2]oxaborinin-6-yl)amino)-5-methylpyrimidin-4-yl)amino)tetrahydro-2H-pyran-4-carbonitrile

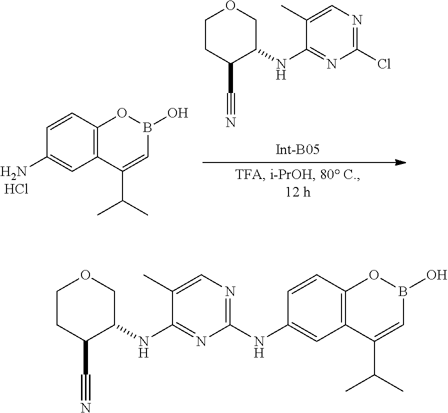

A mixture of 6-amino-4-isopropyl-2H-benzo[e][1,2]oxaborinin-2-ol (0.6 g, 2.51 mmol, 1 eq, HCl), (trans)-3-((2-chloro-5-methylpyrimidin-4-yl)amino)tetrahydro-2H-pyran-4-carbonitrile (633 mg, 2.51 mmol, 1 eq) and TFA (428 mg, 3.76 mmol, 278 uL, 1.5 eq) in i-PrOH (10 mL) was heated to 80° C. and stirred for 12 h. LCMS showed the reaction was complete and desired MS observed. The reaction mixture was cooled to 25° C. and quenched with H₂O (50 mL) and then extracted with EtOAc (50 mL×2). The combined organic phases were washed with brine (50 mL), dried over Na₂SO₄, filtered, and concentrated under reduced pressure to give a residue. The residue was purified by silica column to give (trans)-3-((2-((2-hydroxy-4-isopropyl-2H-benzo[e][1,2]oxaborinin-6-yl)amino)-5-methylpyrimidin-4-yl)amino)tetrahydro-2H-pyran-4-carbonitrile as a white solid (954 mg, 96.10% purity). ¹H NMR: (400 MHz, DMSO-d₆) δ=10.35 (s, 1H), 8.90 (s, 1H), 8.38-8.23 (m, 1H), 7.83-7.71 (m, 2H), 7.50 (dd, J=2.0, 8.8 Hz, 1H), 7.29 (d, J=8.4 Hz, 1H), 6.04 (s, 1H), 4.40-4.27 (m, 1H), 3.93-3.73 (m, 2H), 3.30-3.16 (m, 4H), 2.23-2.11 (m, 1H), 2.04 (s, 3H), 1.83-1.69 (m, 1H), 1.24-1.19 (m, 6H). MS (ESI): mass calcd. For C₂₂H₂₆BN₅O₃, 419.21, m/z found 420.3[M+H]⁺. HPLC: 96.10% (220 nm), 98.52% (254 nm). 900 mg of this material was separated by SFC (column: DAICEL CHIRALPAK AD (250 mm*30 mm, 10 um); mobile phase: [0.1% NH₃H₂O MEOH]; B %: 35%-35%, 7 min) to give (trans)-3-((2-((2-hydroxy-4-isopropyl-2H-benzo[e][1,2]oxaborinin-6-yl)amino)-5-methylpyrimidin-4-yl)amino)tetrahydro-2H-pyran-4-carbonitrile (172.3 mg, 97.49% purity, 99.92% ee, first peak, Rt=3.457 min) stereoisomer one as a white solid. ¹H NMR (400 MHz, DMSO-d₆) δ=8.87 (s, 1H), 8.61 (s, 1H), 8.07 (s, 1H), 7.81-7.71 (m, 2H), 7.10 (d, J=9.2 Hz, 1H), 6.58 (d, J=9.2 Hz, 1H), 5.94 (s, 1H), 4.45-4.48 (m, 1H), 3.84-3.87 (m, 2H), 3.29-3.08 (m, 4H), 2.32-2.15 (m, 1H), 1.91 (s, 3H), 1.87-1.73 (m, 1H), 1.26-1.21 (m, 6H) MS (ESI): mass calcd. For C₂₂H₂₆BN₅O₃, 419.21, m/z found 420.2[M+H]⁺. HPLC: 97.49% (220 nm), 98.76% (254 nm) and (trans)-3-((2-((2-hydroxy-4-isopropyl-2H-benzo[e][1,2]oxaborinin-6-yl)amino)-5-methylpyrimidin-4-yl)amino)tetrahydro-2H-pyran-4-carbonitrile (170.5 mg, 97.45% purity, 98.50% ee, second peak, Rt=3.902 min) stereoisomer two as a white solid. ¹H NMR (400 MHz, DMSO-d₆) δ=8.83 (s, 1H), 8.62 (s, 1H), 8.08 (s, 1H), 7.76-7.74 (m, 2H), 7.13 (d, J=8.8 Hz, 1H), 6.59 (d, J=8.8 Hz, 1H), 5.94 (s, 1H), 4.48-4.42 (m, 1H), 3.88-3.84 (m, 2H), 3.29-3.12 (m, 4H), 2.23-2.14 (m, 1H), 1.98-1.92 (m, 3H), 1.88-1.76 (m, 1H), 1.26-1.21 (m, 6H). MS (ESI): mass calcd. For C₂₂H₂₆BN₅O₃, 419.21, m/z found 420.3[M+H]. HPLC: 97.45% (220 nm), 98.66% (254 nm).

### 40. Preparation of (trans)-3-((2-((8-chloro-2-hydroxy-4-isopropyl-2H-benzo[e][1,2]oxaborinin-6-yl)amino)-5-methylpyrimidin-4-yl)amino)tetrahydro-2H-pyran-4-carbonitrile

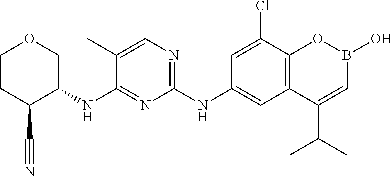 [SMILES: Cc1cnc(Nc2cc(Cl)c3c(c2)C(C(C)C)=CB(O)O3)nc1N[C@H]1COCC[C@@H]1C#N]

### 40.1 Preparation of 2-chlorophenyl isobutyrate

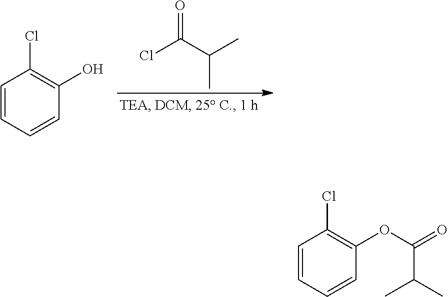

Two reactions were carried out in parallel. For each a solution of 2-chlorophenyl (12 g, 93.3 mmol, 9.5 mL, 1 eq) in DCM (120 mL) was added TEA (18.9 g, 186 mmol, 25.9 mL, 2 eq) and 2-methylpropanoyl chloride (10.9 g, 102 mmol, 10.7 mL, 1.1 eq). The mixture was stirred at 25° C. for 1 h. TLC showed the reaction was complete. The two parallel reactions were combined and diluted with H₂O (100 mL) and extracted with EtOAc (50 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=10:1 to 5:1) to give 2-chlorophenyl isobutyrate (26.3 g, 70.9% yield) as colorless oil. ¹H NMR (400 MHz, DMSO-d₆) δ=7.57 (dd, J=1.6, 8.0 Hz, 1H), 7.43-7.36 (m, 1H), 7.30 (t, J=8.0 Hz, 1H), 2.95-2.80 (m, 1H), 1.27 (d, J=7.6 Hz, 6H).

### 40.2 Preparation of 1-(3-chloro-2-hydroxyphenyl)-2-methylpropan-1-one

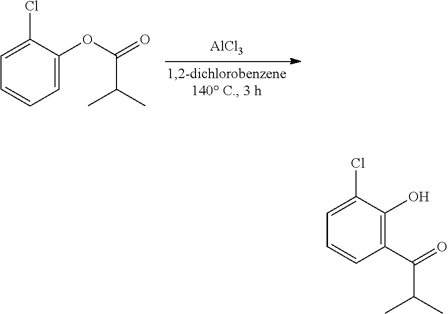

To a solution of AlCl₃ (10 g, 75.5 mmol, 4.13 mL, 1.5 eq) in 1,2-dichlorobenzene (131 g, 891 mmol, 100 mL, 17.7 eq) was added 2-chlorophenyl isobutyrate (10 g, 50.3 mmol, 1 eq) at 25° C. The mixture was stirred at 140° C. for 3 h. TLC showed the reaction was complete. The reaction mixture was diluted with ice-water (100 mL) at 0° C. and extracted with EtOAc (50 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=100:1 to 10:1) to give 1-(3-chloro-2-hydroxyphenyl)-2-methylpropan-1-one (3 g, 30% yield) as yellow oil. ¹H NMR (400 MHz, DMSO-d₆) δ=12.79 (s, 1H), 8.00 (d, J=8.0 Hz, 1H), 7.73 (d, J=8.0 Hz, 1H), 7.01 (t, J=8.0 Hz, 1H), 3.80-3.70 (m, 1H), 1.15 (d, J=6.8 Hz, 6H).

### 40.3 Preparation of 1-(3-chloro-2-hydroxy-5-nitrophenyl)-2-methylpropan-1-one

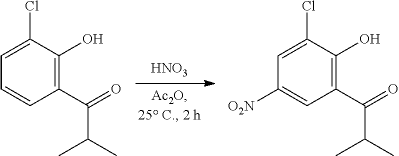

Two reactions were carried out in parallel. For each a mixture of HNO₃ (4.98 g, 51.3 mmol, 3.56 mL, 65% purity, 2.04 eq) and acetyl acetate (5.14 g, 50.3 mmol, 4.71 mL, 2 eq) was stirred at 25° C. for 1 h, then drop-wised added 1-(3-chloro-2-hydroxy-phenyl)-2-methyl-propan-1-one (5 g, 25.1 mmol, 1 eq) at 0° C. The mixture was stirred at 25° C. for 1 h. TLC showed the reaction was complete. The two parallel reactions were combined, diluted with H₂O (50 mL) and extracted with EtOAc (30 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=100:1 to 10:1) to give 1-(3-chloro-2-hydroxy-5-nitro-phenyl)-2-methyl-propan-1-one (8.4 g, 68% yield) as a yellow solid. ¹H NMR (400 MHz, DMSO-d₆) δ=8.63 (d, J=2.8 Hz, 1H), 8.55 (d, J=2.8 Hz, 1H), 3.85-3.75 (m, 1H), 1.16 (d, J=6.8 Hz, 6H).

### 40.4 Preparation of 1-(5-amino-3-chloro-2-hydroxyphenyl)-2-methylpropan-1-one

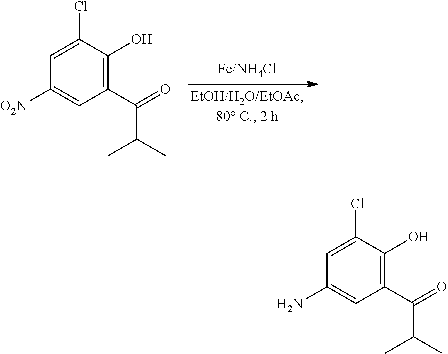 [SMILES: CC(C)C(=O)c1cc([N+](=O)[O-])cc(Cl)c1O ; CC(C)C(=O)c1cc(N)cc(Cl)c1O]

To a solution of 1-(3-chloro-2-hydroxy-5-nitro-phenyl)-2-methyl-propan-1-one (8.3 g, 34.0 mmol, 1 eq) in EtOH (90 mL) and H₂O (30 mL) and EtOAc (90 mL) was added Fe (5.71 g, 102 mmol, 3 eq) and NH₄Cl (5.47 g, 102 mmol, 3 eq) at 25° C. in one portion. The mixture was stirred at 80° C. for 2 hr. TLC showed the reaction was complete. The reaction mixture was concentrated under reduced pressure to give a residue. The residue was quenched with H₂O (100 mL) and extracted with EtOAc (50 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=100:1 to 10:1) to give 1-(5-amino-3-chloro-2-hydroxyphenyl)-2-methylpropan-1-one (2.5 g, 34% yield) as brown oil. ¹H NMR (400 MHz, DMSO-d₆) δ=7.14 (d, J=2.8 Hz, 1H), 7.01 (d, J=2.4 Hz, 1H), 5.04 (s, 2H), 3.60-3.50 (m, 1H), 1.15-1.12 (m, 6H).

### 40.5 Preparation of tert-butyl (3-chloro-4-hydroxy-5-isobutyrylphenyl)carbamate

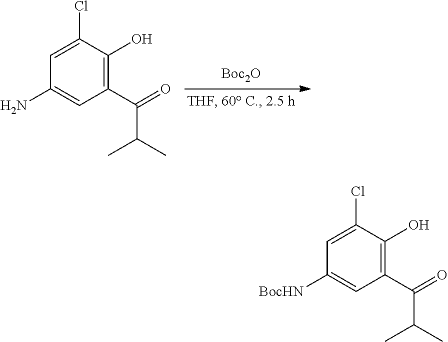 [SMILES: "Boc2O" ; CC(C)C(=O)c1cc(N)cc(Cl)c1O ; CC(C)C(=O)c1cc(NC(=O)OC(C)(C)C)cc(Cl)c1O]

To a solution of 1-(5-amino-3-chloro-2-hydroxyphenyl)-2-methylpropan-1-one (11.6 g, 54.2 mmol, 1 eq) in THE (110 mL) was added Boc₂O (17.7 g, 81.4 mmol, 18.7 mL, 1.5 eq). The mixture was stirred at 60° C. for 2.5 hr. TLC showed the reaction was complete. The reaction mixture was diluted with H₂O (200 mL) and extracted with EtOAc (100 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=5:1 to 3:1) to give tert-butyl N-[3-chloro-4-hydroxy-5-(2-methylpropanoyl)phenyl]carbamate (14 g, 82% yield) as brown oil. ¹H NMR (400 MHz, DMSO-d₆) δ=12.18 (s, 1H), 9.43 (s, 1H), 8.00 (s, 1H), 7.81 (s, 1H), 3.60-3.50 (m, 1H), 1.47 (s, 9H), 1.16 (d, J=6.8 Hz, 6H).

### 40.6 Preparation of tert-butyl (8-chloro-2-hydroxy-4-isopropyl-2H-benzo[e][1,2]oxaborinin-6-yl)carbamate

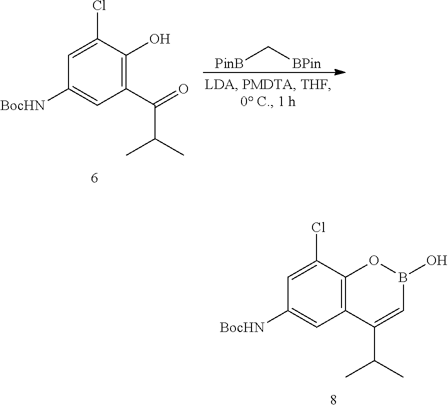

To a solution of LDA (2 M in THF, 59.7 mL, 7.5 eq) in THF (50 mL) was added N-[2-(dimethylamino)ethyl]-N,N,N′-trimethyl-ethane-1,2-diamine (PMDTA, 20.7 g, 119 mmol, 24.9 mL, 7.5 eq) and 4,4,5,5-tetramethyl-2-[(4,4,5,5-tetramethyl-1,3,2-dioxaborolan-2-yl)methyl]-1,3,2-dioxaborolane (29.8 g, 111 mmol, 7 eq) at 0° C., and then the mixture was stirred at 0° C. for 20 min under N₂ atmosphere. Then to above mixture was drop-wise added a solution of tert-butyl N-[3-chloro-4-hydroxy-5-(2-methylpropanoyl)phenyl]carbamate (5 g, 15.9 mmol, 1 eq) in THF (50 mL) at 0° C. The mixture was stirred at 0° C. for 40 min under N₂ atmosphere. TLC showed the reaction was complete. The reaction mixture was diluted with NH₄Cl (200 mL) and extracted with EtOAc (100 mL×3). The combined organic layers were washed with brine (50 mL), dried over anhydrous Na₂SO₄, filtered, and concentrated under reduced pressure to give a residue. The residue was purified by silica gel column chromatography (petroleum ether:EtOAc=100:1 to 10:1) to give tert-butyl (8-chloro-2-hydroxy-4-isopropyl-2H-benzo[e][1,2]oxaborinin-6-yl)carbamate (1.4 g, 26% yield) as a yellow solid. ¹H NMR (400 MHz, DMSO-d₆) δ=9.45 (s, 1H), 9.03 (s, 1H), 7.81 (s, 1H), 7.73 (s, 1H), 6.03 (s, 1H), 3.20-3.00 (m, 1H), 1.48 (s, 9H), 1.15 (s, 6H).

### 40.7 Preparation of 6-amino-8-chloro-4-isopropyl-2H-benzo[e][1,2]oxaborinin-2-ol hydrochloride

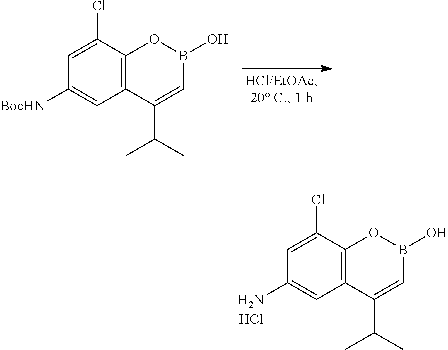 [SMILES: CC(C)C1=CB(O)Oc2c(Cl)cc(NC(=O)OC(C)(C)C)cc21 ; Cl ; CC(C)C1=CB(O)Oc2c(Cl)cc(N)cc21]

To a solution of tert-butyl (8-chloro-2-hydroxy-4-isopropyl-2H-benzo[e][1,2]oxaborinin-6-yl)carbamate (2 g, 5.92 mmol, 1 eq) in EtOAc (10 mL) was added HCl/EtOAc (4 M, 21 mL, 14.2 eq) at 20° C. The mixture was stirred at 20° C. for 1 h. TLC showed the reaction was complete. The reaction mixture was concentrated under reduced pressure to give 6-amino-8-chloro-4-isopropyl-2H-benzo[e][1,2]oxaborinin-2-ol hydrochloride (1.1 g, 67% yield, HCl salt) as a yellow solid. ¹H NMR (400 MHz, DMSO-d₆) δ=10.09 (m, 3H), 7.75 (d, J=2.0 Hz, 1H), 7.56 (d, J=2.0 Hz, 1H), 6.13 (s, 1H), 3.19-3.05 (m, 1H), 1.22 (d, J=6.8 Hz, 6H).

### 40.8a Preparation of (trans)-3-((2-((8-chloro-2-hydroxy-4-isopropyl-2H-benzo[e][1,2]oxaborinin-6-yl)amino)-5-methylpyrimidin-4-yl)amino)tetrahydro-2H-pyran-4-carbonitrile

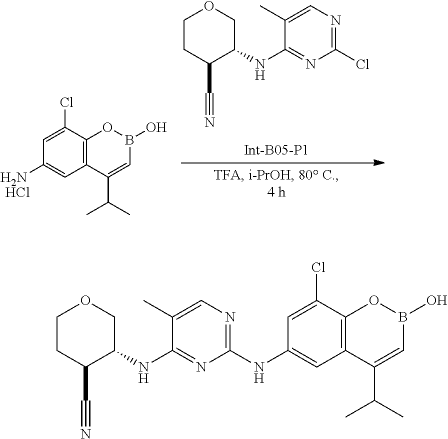 [SMILES: Cl ; CC(C)C1=CB(O)Oc2c(Cl)cc(N)cc21 ; Cc1cnc(Cl)nc1N[C@H]1COCC[C@@H]1C#N ; Cc1cnc(Nc2cc(Cl)c3c(c2)C(C(C)C)=CB(O)O3)nc1N[C@H]1COCC[C@@H]1C#N]

To a solution of 6-amino-8-chloro-4-isopropyl-2H-benzo[e][1,2]oxaborinin-2-ol hydrochloride (300 mg, 1.10 mmol, 1 eq, HCl salt) in i-PrOH (3 mL) was added TFA (188 mg, 1.65 mmol, 0.12 mL, 1.5 eq) and (trans)-3-((2-chloro-5-methylpyrimidin-4-yl)amino)tetrahydro-2H-pyran-4-carbonitrile (250 mg, 990 umol, 0.9 eq). The mixture was stirred at 80° C. for 4 hr. TLC showed the reaction was complete. The reaction mixture was diluted with H₂O (20 mL) and extracted with EtOAc (30 mL×3). The residue was purified by prep-TLC (SiO₂, Petroleum ether:EtOAc=5:1) to give a crude product. The crude product was purified by prep-HPLC (column: Phenomenex Luna C18 75*30 mm*3 um; mobile phase: [water (formic acid-FA)-ACN]; B %: 20%-50%, 8 min) to give (trans)-3-((2-((8-chloro-2-hydroxy-4-isopropyl-2H-benzo[e][1,2]oxaborinin-6-yl)amino)-5-methylpyrimidin-4-yl)amino)tetrahydro-2H-pyran-4-carbonitrile (388 mg, 77% yield, 96.16% purity, 100% ee, first peak, Rt=2.008 min) stereoisomer one as a white solid. ¹H NMR (400 MHz, DMSO-d₆) δ=9.45 (s, 1H), 9.03 (s, 1H), 8.09 (s, 1H), 7.89 (d, J=1.6 Hz, 1H), 7.78 (s, 1H), 7.15 (d, J=5.0 Hz, 1H), 6.04 (s, 1H), 4.53-4.40 (m, 1H), 3.92-3.79 (m, 2H), 3.27-3.12 (m, 4H), 2.18 (d, J=10.0 Hz, 1H), 1.97 (s, 3H), 1.90-1.80 (m, 1H), 1.22 (d, J=6.8 Hz, 6H). MS (ESI): mass calcd. For C₂₂H₂₅BClN₅O₃ 453.17, m/z found 452.0 [M−H]⁻. HPLC: 96.16% (220 nm), 98.11% (254 nm).

### 40.8b Preparation of (trans)-3-((2-((8-chloro-2-hydroxy-4-isopropyl-2H-benzo[e][1,2]oxaborinin-6-yl)amino)-5-methylpyrimidin-4-yl)amino)tetrahydro-2H-pyran-4-carbonitrile

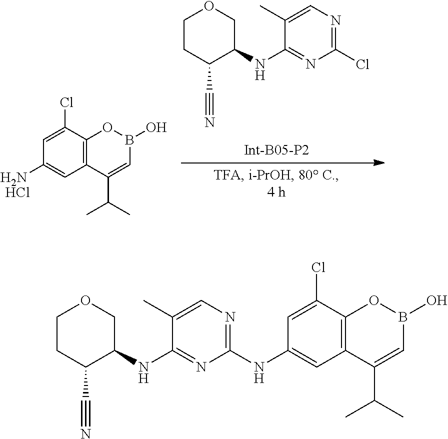 [SMILES: Cl ; CC(C)C1=CB(O)Oc2c(Cl)cc(N)cc21 ; Cc1cnc(Cl)nc1N[C@@H]1COCC[C@H]1C#N ; Cc1cnc(Nc2cc(Cl)c3c(c2)C(C(C)C)=CB(O)O3)nc1N[C@@H]1COCC[C@H]1C#N]

To a solution of 6-amino-8-chloro-4-isopropyl-2H-benzo[e][1,2]oxaborinin-2-ol hydrochloride (300 mg, 1.10 mmol, 1 eq, HCl) in i-PrOH (3 mL) was added TFA (188 mg, 1.65 mmol, 122 uL, 1.5 eq) and (trans)-3-((2-chloro-5-methylpyrimidin-4-yl)amino)tetrahydro-2H-pyran-4-carbonitrile (249 mg, 985 umol, 0.9 eq). The mixture was stirred at 80° C. for 4 hr. TLC showed the reaction was complete. The reaction mixture was diluted with H₂O (20 mL) and extracted with EtOAc (10 mL×3). The residue was purified by prep-TLC (SiO₂, petroleum ether:EtOAc=5:1) to give a crude product. The crude product was purified by prep-HPLC (column: Phenomenex Luna C18 75*30 mm*3 um; mobile phase: [water (formic acid-FA)-ACN]; B %: 20%-50%, 8 min) to give (trans)-3-((2-((8-chloro-2-hydroxy-4-isopropyl-2H-benzo[e][1,2]oxaborinin-6-yl)amino)-5-methylpyrimidin-4-yl)amino)tetrahydro-2H-pyran-4-carbonitrile (310 mg, 62% yield, 98.80% purity, 97.52% ee, second peak, Rt=2.397 min), stereoisomer two as a white solid. ¹H NMR (400 MHz, DMSO-d₆) δ=9.38 (s, 1H), 9.02 (s, 1H), 8.10 (s, 1H), 7.89 (d, J=2.0 Hz, 1H), 7.78 (s, 1H), 7.08 (s, 1H), 6.04 (s, 1H), 4.52-4.41 (m, 1H), 3.92-3.79 (m, 2H), 3.28-3.10 (m, 4H), 2.22-2.13 (m, 1H), 1.97 (s, 3H), 1.83-1.80 (m, 1H), 1.23-1.21 (d, J=6.8 Hz, 6H). MS (ESI): mass calcd. For C₂₂H₂₅BClN₅O₃ 453.17, m/z found 452.0 [M−H]⁻. HPLC: 98.80% (220 nm), 99.47% (254 nm).

### 41. Preparation of (trans)-3-((5-chloro-2-((1-hydroxy-7-methyl-1,3-dihydrobenzo [c][1,2]oxaborol-5-yl)amino)pyrimidin-4-yl)amino)tetrahydro-2H-pyran-4-carbonitrile

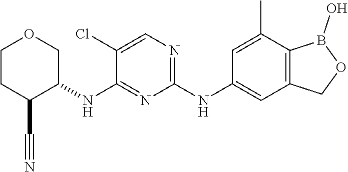 [SMILES: Cc1cc(Nc2ncc(Cl)c(N[C@H]3COCC[C@@H]3C#N)n2)cc2c1B(O)OC2]

### 41.1 Preparation of methyl 2-bromo-3-methyl-5-nitrobenzoate

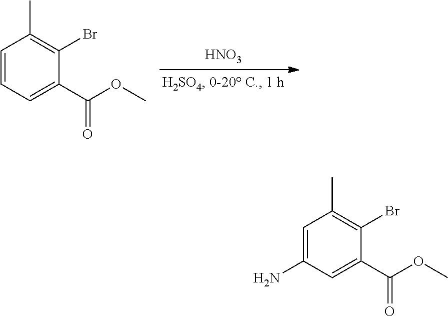

To a solution of methyl 2-bromo-3-methyl-benzoate (20 g, 87.3 mmol, 1 eq) in H2SO4 (98% purity, 200 mL) was added HNO3 (19.2 g, 275 mmol, 90% purity, 3.15 eq) at 0° C. The mixture was stirred at 20° C. for 1 hr. LCMS showed the starting material was consumed completely and desired MS was detected. The reaction mixture was poured into ice-water (200 mL) at 0° C., and large amount of solid formed and was collected by filtration to give a crude product. The crude product was purified by silica gel column chromatography (petroleum ether:ethyl acetate (EtOAc)=100:1 to 10:1) and triturated with DCM (20 mL) at 25° C. for 30 min to give methyl 2-bromo-3-methyl-5-nitro-benzoate (18 g, 75.2% yield) as a white solid. 1H NMR (400 MHz, CDCl3) δ=8.35 (d, J=2.8 Hz, 1H), 8.21 (d, J=2.8 Hz, 1H), 4.00 (s, 3H), 2.60 (s, 3H).

### 41.2 Preparation of methyl 5-amino-2-bromo-3-methylbenzoate

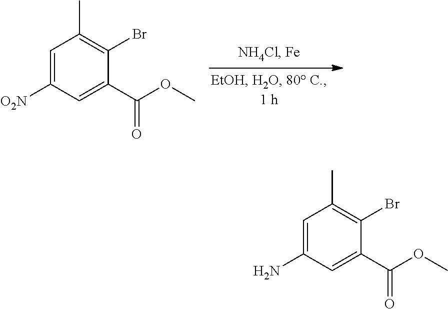

To a solution of methyl 2-bromo-3-methyl-5-nitro-benzoate (14.7 g, 53.6 mmol, 1 eq) in EtOH (150 mL) and H₂O (30 mL) was added Fe (12 g, 214 mmol, 4 eq) and NH₄Cl (12 g, 214 mmol, 4 eq) at 25° C. The mixture was stirred at 80° C. for 1 hr. LCMS showed the starting material was consumed completely and desired MS was detected. The reaction mixture was diluted with H₂O (300 mL) and extracted with ethyl acetate (100 mL×3). The combined organic layers were washed with brine (100 mL×3), dried over Na₂SO₄, filtered, and concentrated under reduced pressure to give methyl 5-amino-2-bromo-3-methyl-benzoate (12.3 g, 93.9% yield) as yellow oil. ¹H NMR (400 MHz, DMSO-d₆) δ=6.66 (s, 2H), 5.43 (s, 2H), 3.79 (s, 3H), 2.23 (s, 3H).

### 41.3 Preparation of (5-amino-2-bromo-3-methylphenyl)methanol

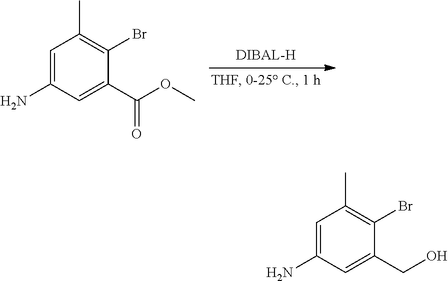

To a solution of methyl 5-amino-2-bromo-3-methyl-benzoate (5 g, 20.5 mmol, 1 eq) in THF (50 mL) was drop-wise added DIBAL-H (1 M in THF, 82 mL, 4 eq) at 0° C. After the addition, the reaction was allowed to warm to 25° C. and stirred for 1 hr. LCMS showed the starting material was consumed completely and desired MS was detected. The reaction mixture was quenched with saturated aq. seignette salt (100 mL) and extracted with EtOAc (50×3 mL). The combined organic layers were washed with brine (50 mL), dried over anhydrous Na₂SO₄, filtered, and concentrated under reduced pressure to give (5-amino-2-bromo-3-methyl-phenyl)methanol (1 g, 22.5% yield) as an off-white solid. ¹H NMR (400 MHz, DMSO-d₆) δ=6.64 (d, J=2.4 Hz, 1H), 6.41 (d, J=2.4 Hz, 1H), 5.21 (t, J=5.6 Hz, 1H), 5.17-5.07 (m, 2H), 4.36 (d, J=5.6 Hz, 2H), 2.18 (s, 3H).

### 41.4 Preparation of (trans)-3-((2-((4-bromo-3-(hydroxymethyl)-5-methylphenyl)amino)-5-chloropyrimidin-4-yl)amino)tetrahydro-2H-pyran-4-carbonitrile

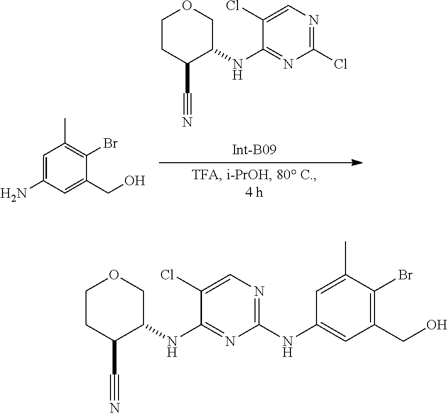

To a solution of (5-amino-2-bromo-3-methyl-phenyl)methanol (900 mg, 4.17 mmol, 1 eq) in i-PrOH (9 mL) was added TFA (713 mg, 6.26 mmol, 463 uL, 1.5 eq) and (trans)-3-[(2,5-dichloropyrimidin-4-yl)amino]tetrahydropyran-4-carbonitrile (1 g, 3.75 mmol, 0.9 eq) at 25° C. The mixture was stirred at 80° C. for 4 h. LCMS showed the starting material was consumed completely and desired MS was detected. The reaction mixture was concentrated under reduced pressure to give a crude product. The crude product was triturated with MTBE (10 mL) at 25° C. for 20 min to give (trans)-3-((2-((4-bromo-3-(hydroxymethyl)-5-methylphenyl)amino)-5-chloropyrimidin-4-yl)amino)tetrahydro-2H-pyran-4-carbonitrile (1.5 g, 79.4% yield) as an off-white solid. ¹H NMR (400 MHz, DMSO-d₆) δ=10.38 (s, 1H), 8.33-8.20 (m, 2H), 7.70-7.60 (m, 2H), 4.54-4.40 (m, 3H), 3.90-3.80 (m, 2H), 3.80-3.70 (m, 1H), 3.50-3.40 (m, 1H), 3.30-3.20 (m, 2H), 2.37 (s, 3H), 2.22-2.11 (m, 1H), 1.90-1.80 (m, 1H).

### 41.5 Preparation of (trans)-3-((5-chloro-2-((1-hydroxy-7-methyl-1,3-dihydrobenzo [c][1,2]oxaborol-5-yl)amino)pyrimidin-4-yl)amino)tetrahydro-2H-pyran-4-carbonitrile

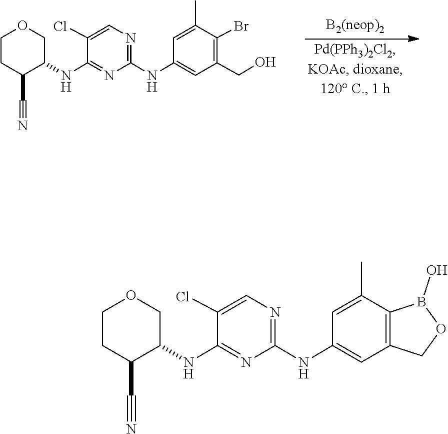 [SMILES: Cc1cc(Nc2ncc(Cl)c(N[C@H]3COCC[C@@H]3C#N)n2)cc(CO)c1Br ; Cc1cc(Nc2ncc(Cl)c(N[C@H]3COCC[C@@H]3C#N)n2)cc2c1B(O)OC2]

Two reactions were carried out in parallel. For each a mixture of (trans)-3-((2-((4-bromo-3-(hydroxymethyl)-5-methylphenyl)amino)-5-chloropyrimidin-4-yl)amino)tetrahydro-2H-pyran-4-carbonitrile (500 mg, 1.10 mmol, 1 eq), 2-(5,5-dimethyl-1,3,2-dioxaborinan-2-yl)-5,5-dimethyl-1,3,2-dioxaborinane (994 mg, 4.40 mmol, 4 eq), KOAc (324 mg, 3.30 mmol, 3 eq), Pd(PPh₃)₂Cl₂ (77.2 mg, 110 umol, 0.1 eq) in dioxane (10 mL) was degassed and purged with N₂ for 3 times, and then the mixture was stirred at 120° C. for 1 hr under N₂ atmosphere. LCMS showed the reaction was complete and desired MS observed. The two parallel reactions were combined and filtered. The filtrate was concentrated under reduced pressure to give a residue. The residue was purified by prep-HPLC (column: DAICEL CHIRALPAK AD (250 mm*30 mm, 10 um); mobile phase: [0.1% NH₃H₂O IPA]; B %: 35%-35%, 13 min) to give (trans)-3-((5-chloro-2-((1-hydroxy-7-methyl-1,3-dihydrobenzo[c][1,2]oxaborol-5-yl)amino)pyrimidin-4-yl)amino)tetrahydro-2H-pyran-4-carbonitrile (300 mg, 34.0% yield) as an off-white solid. ¹H NMR (400 MHz, DMSO-d₆) δ=9.48 (s, 1H), 8.67 (s, 1H), 8.06 (s, 1H), 7.61 (s, 1H), 7.41 (s, 1H), 7.32 (d, J=8.8 Hz, 1H), 4.95-4.85 (m, 2H), 4.50-4.40 (m, 1H), 3.94-3.83 (m, 2H), 3.40-3.30 (m, 1H), 3.31-3.24 (m, 2H), 3.18 (t, J=10.8 Hz, 1H), 2.39 (s, 3H), 2.20-2.10 (m, 1H), 1.85-1.74 (m, 1H). This material was separated by SFC (column: DAICEL CHIRALPAK AD (250 mm*30 mm, 10 um); mobile phase: [0.1% NH₃H₂O IPA]; B %: 35%-35%, 13 min) to give (trans)-3-[[5-chloro-2-[(1-hydroxy-7-methyl-3H-2,1-benzoxaborol-5-yl)amino]pyrimidin-4-yl]amino]tetrahydropyran-4-carbonitrile (63.4 mg, 98.73% purity, 100% ee, first peak, Rt=1.453 min), stereoisomer one as a white solid. ¹H NMR (400 MHz, DMSO-d₆) δ=9.47 (s, 1H), 8.67 (s, 1H), 8.06 (s, 1H), 7.61 (s, 1H), 7.41 (s, 1H), 7.31 (d, J=8.4 Hz, 1H), 4.89 (s, 2H), 4.50-4.40 (m, 1H), 3.94-3.83 (m, 2H), 3.40-3.30 (m, 1H), 3.31-3.24 (m, 1H), 3.20-3.10 (m, 1H), 2.39 (s, 3H), 2.20-2.10 (m, 1H), 1.90-1.77 (m, 1H). MS (ESI): mass calcd. For C₁₈H₁₉BClN₅O₃ 399.13, m/z found 400.2 [M+H]⁺. HPLC: 98.73% (220 nm), 99.46% (254 nm). and (trans)-3-[[5-chloro-2-[(1-hydroxy-7-methyl-3H-2,1-benzoxaborol-5-yl)amino]pyrimidin-4-yl]amino]tetrahydropyran-4-carbonitrile (94.7 mg, 96.68% purity, 99.64% ee, second peak, Rt=1.864 min), stereoisomer two as a white solid. ¹H NMR (400 MHz, DMSO-d₆) δ=9.48 (s, 1H), 8.67 (s, 1H), 8.06 (s, 1H), 7.61 (s, 1H), 7.41 (s, 1H), 7.32 (d, J=8.8 Hz, 1H), 4.95-4.85 (m, 2H), 4.50-4.37 (m, 1H), 3.97-3.82 (m, 2H), 3.40-3.30 (m, 1H), 3.30-3.23 (m, 1H), 3.18 (t, J=10.8 Hz, 1H), 2.40 (s, 3H), 2.20-2.10 (m, 1H), 1.90-1.77 (m, 1H). MS (ESI): mass calcd. For C₁₈H₁₉BClN₅O₃ 399.13, m/z found 400.1 [M+H]⁺. HPLC: 96.68% (220 nm), 95.86% (254 nm).

### 42. Preparation of (trans)-3-((5-chloro-2-((1-hydroxy-7-(trifluoromethyl)-1,3-dihydrobenzo[c][1,2]oxaborol-5-yl)amino)pyrimidin-4-yl)amino)tetrahydro-2H-pyran-4-carbonitrile

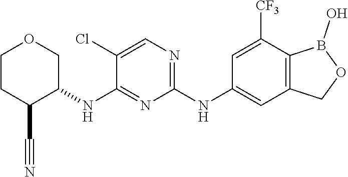 [SMILES: N#C[C@H]1CCOC[C@@H]1Nc1nc(Nc2cc3c(c(C(F)(F)F)c2)B(O)OC3)ncc1Cl]

### 42.1 Preparation of (5-amino-2-bromo-3-(trifluoromethyl)phenyl)methanol

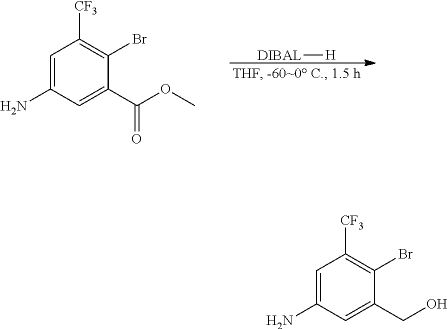

To a solution of methyl 5-amino-2-bromo-3-(trifluoromethyl)benzoate (5 g, 16.8 mmol, 1 eq) in THF (100 mL) was added DIBAL-H (1 M in THF, 42 mL, 2.5 eq) dropwise at −60° C. The mixture was stirred at −60° C. for 30 min. Then the mixture was stirred at 0° C. for 1 h. TLC showed the reaction was complete. The reaction mixture was quenched by addition of aq. seignette salt (100 mL) at 0° C., and then extracted with EtOAc (100 mL×3). The combined organic layers were washed with brine (100 mL×2), dried over Na₂SO₄, filtered, and concentrated under reduced pressure to give a residue. The residue was purified by flash silica gel chromatography (ISCO®; 80 g SepaFlash® Silica Flash Column, Eluent of 0˜50% Ethyl acetate/petroleum ether gradient @ 75 mL/min) to give (5-amino-2-bromo-3-(trifluoromethyl) phenyl) methanol (3.2 g, 70.6% yield) as a yellow solid. ¹H NMR (CDCl₃, 400 MHz) δ=7.01 (d, J=2.4 Hz, 1H), 6.94 (d, J=2.8 Hz, 1H), 4.74 (s, 2H).

### 42.2 Preparation of (trans)-3-((2,5-dichloropyrimidin-4-yl)amino)tetrahydro-2H-pyran-4-carbonitrile

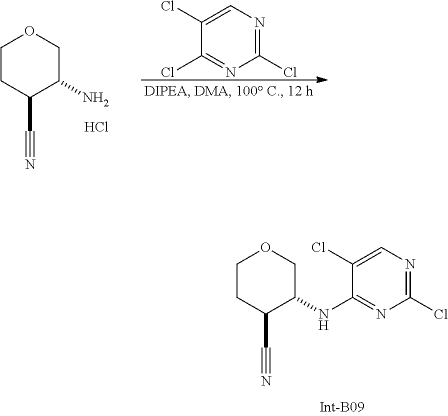 [SMILES: N#C[C@H]1CCOC[C@@H]1N ; Cl ; Clc1ncc(Cl)c(Cl)n1 ; N#C[C@H]1CCOC[C@@H]1Nc1nc(Cl)ncc1Cl]

To a solution of (trans)-3-aminotetrahydropyran-4-carbonitrile (3.4 g, 20.9 mmol, 1 eq, HCl) in dioxane (40 mL) was added 2,4,5-trichloropyrimidine (3.07 g, 16.7 mmol, 0.8 eq), DIPEA (5.4 g, 41.8 mmol, 7.3 mL, 2 eq) at 25° C. The mixture was stirred at 100° C. for 12 hr. LCMS showed the reaction was complete and desired MS observed. The reaction mixture was poured into saturated aq. NH₄Cl (40 mL), extracted with EtOAc (40 mL×3) and the combined organic layers were washed with brine (40 mL×2), dried over Na₂SO₄, filtered, and concentrated under reduced pressure to give a residue. The residue was purified by flash silica gel chromatography (ISCO®; 80 g SepaFlash® Silica Flash Column, eluent of 0˜25% ethyl acetate/petroleum ether gradient @ 50 mL/min) to give (trans)3-[(2,5-dichloropyrimidin-4-yl)amino] tetrahydropyran-4-carbonitrile (1.7 g, 29.8% yield) as a yellow solid. ¹H NMR (DMSO-d₆, 400 MHz) δ=8.13 (s, 1H), 5.97 (d, J=6.8 Hz, 1H), 4.40 (d, J=3.2 Hz, 1H), 4.11 (dd, J=2.4, 12.4 Hz, 1H), 3.89 (dd, J=3.2, 7.6 Hz, 2H), 3.81 (dd, J=2.4, 12.4 Hz, 1H), 3.28-3.25 (m, 1H), 2.19-2.11 (m, 1H), 1.90-1.85 (m, 1H).

### 42.3 Preparation of 3-((2-((4-bromo-3-(hydroxymethyl)-5-(trifluoromethyl)phenyl) amino)-5-chloropyrimidin-4-yl)amino)tetrahydro-2H-pyran-4-carbonitrile

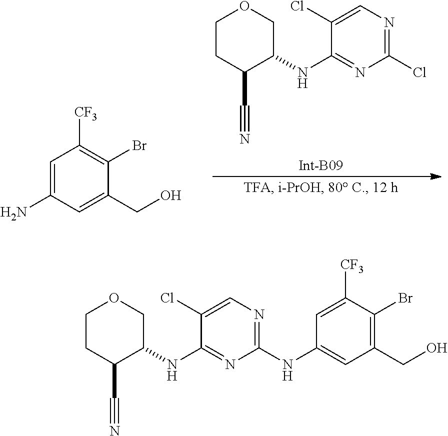 [SMILES: Nc1cc(CO)c(Br)c(C(F)(F)F)c1 ; N#C[C@H]1CCOC[C@@H]1Nc1nc(Cl)ncc1Cl ; N#C[C@H]1CCOC[C@@H]1Nc1nc(Nc2cc(CO)c(Br)c(C(F)(F)F)c2)ncc1Cl]

To a mixture of (5-amino-2-bromo-3-(trifluoromethyl) phenyl) methanol (1.6 g, 5.9 mmol, 1 eq) and (trans)-3-[(2,5-dichloropyrimidin-4-yl)amino]tetrahydropyran-4-carbonitrile (1.62 g, 5.9 mmol, 1 eq) in i-PrOH (20 mL) was added TFA (1.35 g, 11.9 mmol, 877 uL, 2 eq) in one portion at 25° C. The mixture was stirred at 25° C. for 10 min, then heated to 80° C. and stirred for 12 hours. LCMS showed the reaction was complete and desired MS observed. The reaction mixture was filtered and the filter cake was washed with saturated aq. NaHCO₃ (10 mL) to give a residue. The residue was triturated with MTBE (10 mL) at 25° C. for 30 min to give (trans)-3-((2-((4-bromo-3-(hydroxymethyl)-5-(trifluoromethyl)phenyl)amino)-5-chloropyrimidin-4-yl)amino)tetrahydro-2H-pyran-4-carbonitrile (2.23 g, 74.2% yield) as a white solid. ¹H NMR (CDCl₃, 400 MHz) δ=8.26 (s, 1H), 8.02 (s, 1H), 7.67 (s, 1H), 7.25 (s, 1H), 5.72 (d, J=8.0 Hz, 1H), 4.84 (s, 2H), 4.49-4.46 (m, 1H), 4.20-4.16 (m, 1H), 3.96-3.90 (m, 1H), 3.84-3.79 (m, 1H), 3.67 (dd, J=4.8, 12.4 Hz, 1H), 3.17-3.13 (m, 1H), 2.23-2.15 (m, 1H), 1.96-1.87 (m, 1H), 1.59-1.52 (m, 1H).

### 42.4 Preparation of 3-((2-((4-bromo-3-(((tert-butyldimethylsilyl)oxy)methyl)-5-(trifluoromethyl)phenyl)amino)-5-chloropyrimidin-4-yl)amino)tetrahydro-2H-pyran-4-carbonitrile

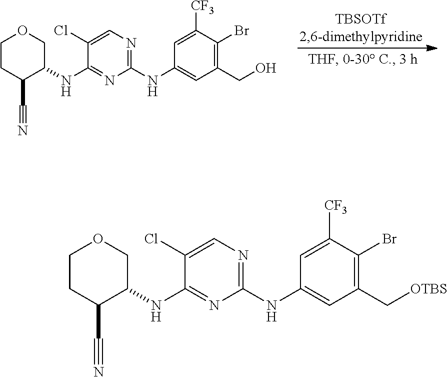 [SMILES: N#C[C@H]1CCOC[C@@H]1Nc1nc(Nc2cc(CO)c(Br)c(C(F)(F)F)c2)ncc1Cl ; CC(C)(C)[Si](C)(C)OCc1cc(Nc2ncc(Cl)c(N[C@H]3COCC[C@@H]3C#N)n2)cc(C(F)(F)F)c1Br]

To a solution of (trans)-3-((2-((4-bromo-3-(hydroxymethyl)-5-(trifluoromethyl)phenyl)amino)-5-chloropyrimidin-4-yl)amino)tetrahydro-2H-pyran-4-carbonitrile (2 g, 3.95 mmol, 1 eq) and 2,6-dimethylpyridine (1.06 g, 9.90 mmol, 1.2 mL, 2.5 eq) in THF (40 mL) was added TBSOTf (1.57 g, 5.9 mmol, 1.4 mL, 1.5 eq) at 0° C. The mixture was stirred at 30° C. for 3 hr. LCMS showed the reaction was complete and desired MS observed. The reaction mixture was quenched by addition saturated aq. NH₄Cl (50 mL) at 0° C., and then extracted with EtOAc (30 mL×3) and the combined organic layers were washed with brine (50 mL×2), dried over Na₂SO₄, filtered, and concentrated under reduced pressure to give a residue. The residue was purified by column chromatography (SiO₂, petroleum ether/ethyl acetate=10/1 to 0/1) to give (trans)-3-((2-((4-bromo-3-(((tert-butyldimethylsilyl)oxy)methyl)-5-(trifluoromethyl)phenyl) amino)-5-chloropyrimidin-4-yl)amino)tetrahydro-2H-pyran-4-carbonitrile (971 mg, 39.6% yield) as a white solid. ¹H NMR (CDCl₃, 400 MHz) δ=9.95 (s, 1H), 8.37 (d, J=2.4 Hz, 1H), 8.06 (s, 2H), 7.38 (d, J=8.8 Hz, 1H), 4.73 (s, 2H), 4.47-4.44 (m, 1H), 3.90-3.81 (m, 2H), 3.36-3.16 (m, 3H), 2.19-2.16 (m, 1H), 1.82-1.78 (m, 1H), 0.95 (s, 9H), 0.14 (s, 6H).

### 42.5 Preparation of 3-((2-((3-(((tert-butyldimethylsilyl)oxy)methyl)-4-(5,5-dimethyl-1,3,2-dioxaborinan-2-yl)-5-(trifluoromethyl)phenyl)amino)-5-chloropyrimidin-4-yl)amino)tetrahydro-2H-pyran-4-carbonitrile

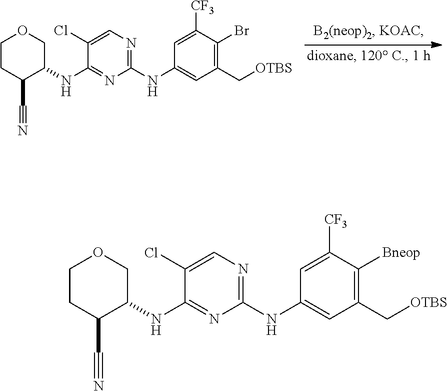

Seven reactions were carried out in parallel. For each a mixture of (trans)-3-((2-((4-bromo-3-(((tert-butyldimethylsilyl)oxy)methyl)-5-(trifluoromethyl)phenyl)amino)-5-chloropyrimidin-4-yl)amino)tetrahydro-2H-pyran-4-carbonitrile (500 mg, 805 umol, 1 eq), 2-(5,5-dimethyl-1,3,2-dioxaborinan-2-yl)-5,5-dimethyl-1,3,2-dioxaborinane (909 mg, 4.03 mmol, 5 eq), KOAc (237 mg, 2.42 mmol, 3 eq), Pd(PPh₃)₂Cl₂ (57 mg, 80.5 umol, 0.1 eq) in dioxane (10 mL) was degassed and purged with N₂ for 3 times, and then the mixture was stirred at 120° C. for 1 hr under N₂ atmosphere. LCMS showed the reaction was complete and desired MS observed. The seven parallel reactions were combined, filtered, and concentrated under reduced pressure to give a residue. The residue was purified by prep-HPLC (column: Waters Xbridge Prep OBD C₁₈ 150×40 mm×10 um; mobile phase: [water (NH₄HCO₃)-ACN]; B %: 65%-95%, 8 min) to give (trans)-3-((2-((3-(((tert-butyldimethylsilyl)oxy)methyl)-4-(5,5-dimethyl-1,3,2-dioxaborinan-2-yl)-5-(trifluoromethyl)phenyl)amino)-5-chloropyrimidin-4-yl)amino)tetrahydro-2H-pyran-4-carbonitrile (1.2 g, 32.55% yield) as a purple solid.

### 42.6 Preparation of (trans)-3-((5-chloro-2-((1-hydroxy-7-(trifluoromethyl)-1,3-dihydrobenzo[c][1,2]oxaborol-5-yl)amino)pyrimidin-4-yl)amino)tetrahydro-2H-pyran-4-carbonitrile

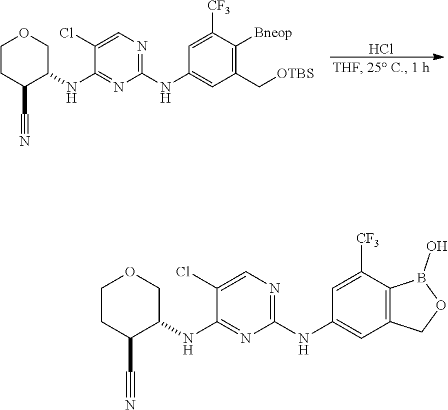

To a solution of (trans)-3-((2-((3-(((tert-butyldimethylsilyl)oxy)methyl)-4-(5,5-dimethyl-1,3,2-dioxaborinan-2-yl)-5-(trifluoromethyl)phenyl)amino)-5-chloropyrimidin-4-yl)amino)tetrahydro-2H-pyran-4-carbonitrile (1.2 g, 1.8 mmol, 1 eq) in THF (12 mL) was added aq. HCl (4 M, 1.8 mL, 4 eq). The mixture was stirred at 25° C. for 1 h. LCMS showed the reaction was complete and desired MS observed. The reaction mixture was concentrated under reduced pressure to give a residue. The crude product was triturated with MeCN (10 mL) at 25° C. for 30 min to give the residue. The residue was purified by prep-HPLC (column: Phenomenex Luna 80×30 mm×3 um; mobile phase: [water (HCl)-ACN]; B %: 10%-40%, 8 min) to give (trans)-3-((5-chloro-2-((1-hydroxy-7-(trifluoromethyl)-1,3-dihydrobenzo[c][1,2]oxaborol-5-yl)amino)pyrimidin-4-yl)amino)tetrahydro-2H-pyran-4-carbonitrile (755 mg, 90.7% yield, 97.88% purity) as a white solid. ¹H NMR (DMSO-d₆, 400 MHz) δ=9.91 (s, 1H), 8.96 (s, 1H), 8.13 (s, 1H), 8.06 (s, 2H), 7.42 (d, J=8.8 Hz, 1H), 5.06-4.97 (m, 2H), 4.49-4.38 (m, 1H), 3.91-3.84 (m, 2H), 3.30-3.24 (m, 2H), 3.18 (t, J=10.8 Hz, 1H), 2.18 (d, J=9.6 Hz, 1H), 1.90-1.80 (m, 1H). MS (ESI): mass calcd. For C₁₈H₁₆BClF₃N₅O₃ 453.10, m/z found 452.0 [M−H]⁻. HPLC: 97.88% (220 nm), 97.77% (254 nm). This material was separated by SFC (column: ChiralPak IH, 250×30 mm, 10 um; mobile phase: [0.1% NH₃H₂O ETOH]; B %: 25%-25%, 6 min) to give (trans)-3-((5-chloro-2-((1-hydroxy-7-(trifluoromethyl)-1,3-dihydrobenzo[c][1,2]oxaborol-5-yl)amino)pyrimidin-4-yl)amino)tetrahydro-2H-pyran-4-carbonitrile (184.1 mg, 99.14% purity, 100% ee, first peak, Rt=1.077 min) stereoisomer one as a white solid. ¹H NMR (DMSO-d₆, 400 MHz) δ=9.91 (s, 1H), 8.95 (s, 1H), 8.13 (s, 1H), 8.10-8.00 (m, 2H), 7.45-7.40 (m, 1H), 5.12-4.90 (m, 2H), 4.52-4.35 (m, 1H), 3.99-3.76 (m, 2H), 3.31-3.23 (m, 2H), 3.20-3.10 (m, 1H), 2.18 (d, J=11.6 Hz, 1H), 1.90-1.80 (m, 1H). MS (ESI): mass calcd. For C₁₈H₁₆BClF₃N₅O₃ 453.10, m/z found 452.0 [M−H]⁻. HPLC: 99.14% (220 nm), 99.06% (254 nm) and (trans)-3-((5-chloro-2-((1-hydroxy-7-(trifluoromethyl)-1,3-dihydrobenzo[c][1,2]oxaborol-5-yl)amino)pyrimidin-4-yl)amino)tetrahydro-2H-pyran-4-carbonitrile (201.5 mg, 99.76% purity, 99.16% ee, second peak, Rt=1.146 min) stereoisomer two as a white solid. ¹H NMR (DMSO-d₆, 400 MHz) δ=9.91 (s, 1H), 8.95 (s, 1H), 8.13 (s, 1H), 8.06 (s, 2H), 7.41 (d, J=8.8 Hz, 1H), 5.12-4.90 (m, 2H), 4.52-4.35 (m, 1H), 3.99-3.76 (m, 2H), 3.31-3.23 (m, 2H), 3.19 (t, J=10.8 Hz, 1H), 2.18 (d, J=11.6 Hz, 1H), 1.90-1.80 (m, 1H). MS (ESI): mass calcd. For C₁₈H₁₆BClF₃N₅O₃ 453.10, m/z found 451.9 [M−H]⁻. HPLC: 99.76% (220 nm), 99.82% (254 nm).

### 43. Preparation of (trans)-4-((2-((1-hydroxy-7-(trifluoromethyl)-1,3-dihydrobenzo [c][1,2]oxaborol-5-yl)amino)-5-methylpyrimidin-4-yl)amino)tetrahydrofuran-3-carbonitrile

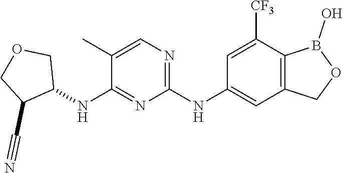 [SMILES: Cc1cnc(Nc2cc3c(c(C(F)(F)F)c2)B(O)OC3)nc1N[C@H]1COC[C@@H]1C#N]

### 43.1 Preparation of 6-tosyl-3-oxa-6-azabicyclo[3.1.0]hexane

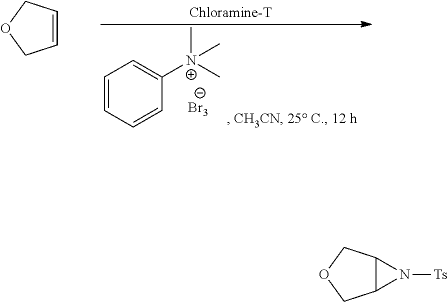

Three reactions were carried out in parallel. For each a mixture of 2,5-dihydrofuran (10 g, 142 mmol, 10.8 mL, 1 eq) and phenyltrimethylammonium tribromide (8.0 g, 21.4 mmol, 0.15 eq) in MeCN (100 mL) was added Chloramine-T (48.7 g, 214 mmol, 1.5 eq) at 25° C., then the reaction mixture was stirred at 25° C. for 12 h. TLC showed the reaction was complete. The three parallel reactions were combined and filtered. The filtrate was concentrated under reduced pressure to give a residue. The residue was purified by prep-HPLC (Phenomenex Titank C18 Bulk 250*150 mm 10 u); mobile phase: [water (NH₄HCO₃)-MeCN]; B %: 30%-60%, 20 min) to give 6-(p-tolylsulfonyl)-3-oxa-6-azabicyclo[3.1.0]hexane (27 g, 26.3% yield) as a white solid. ¹H NMR (400 MHz, CDCl₃) δ 7.85 (d, J=8.0 Hz, 2H), 7.34 (d, J=8.0 Hz, 2H), 3.99 (d, J=10.0 Hz, 2H), 3.73-3.64 (m, 4H), 2.45 (s, 3H).

### 43.2 Preparation of N-((trans)-4-cyanotetrahydrofuran-3-yl)-4-methylbenzenesulfonamide

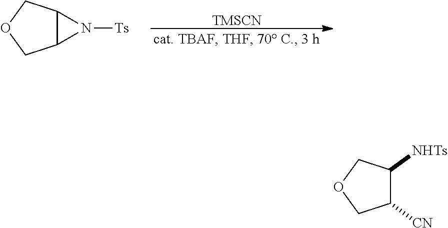

To a solution of 6-(p-tolylsulfonyl)-3-oxa-6-azabicyclo[3.1.0]hexane (26 g, 108 mmol, 1 eq) in THF (150 mL) was drop-wise added TMSCN (32.3 g, 326 mmol, 3 eq) and TBAF (1 M in THF, 43.4 mL, 0.4 eq) at 25° C. under N₂. And then the resulting mixture was stirred at 70° C. for 3 hrs. TLC showed the reaction was complete. The reaction mixture was diluted with H₂O (200 mL) and extracted with EtOAc (200 mL×3). The combined organic layers were washed with brine (500 mL), dried over anhydrous Na₂SO₄, filtered, and concentrated under reduced pressure to give a residue. The residue was purified by silica gel column chromatography (Petroleum ether:EtOAc=100:1 to 1:1) to give N-((trans)-4-cyanotetrahydrofuran-3-yl)-4-methyl-benzenesulfonamide (17 g, 58.7% yield) as a white solid. ¹H NMR (400 MHz, CDCl₃) δ=7.80 (d, J=8.4 Hz, 2H), 7.37 (d, J=8.0 Hz, 2H), 5.89-5.66 (m, 1H), 4.10-4.00 (m, 1H), 3.95-3.85 (m, 2H), 3.70-3.60 (m, 1H), 3.19-3.04 (m, 1H), 2.46 (s, 3H).

### 43.3 Preparation of tert-butyl ((trans)-4-cyanotetrahydrofuran-3-yl)(tosyl)carbamate

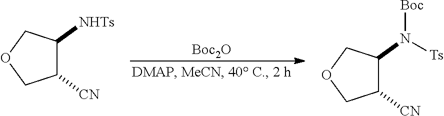

A mixture of N-((trans)-4-cyanotetrahydrofuran-3-yl)-4-methyl-benzenesulfonamide (17 g, 63.8 mmol, 1 eq), Boc₂O (20.9 g, 95.7 mmol, 1.5 eq), DMAP (2.34 g, 19.1 mmol, 0.3 eq) in MeCN (100 mL) was degassed and purged with N₂ for 3 times, and then the mixture was stirred at 40° C. for 2 hrs under N₂ atmosphere. TLC showed the reaction was complete. The reaction mixture was diluted with H₂O (200 mL) and extracted with EtOAc (100×3 mL). The combined organic layers were washed with brine (100 mL), dried over anhydrous Na₂SO₄, filtered, and concentrated under reduced pressure to give a residue. The residue was purified by silica gel column chromatography (petroleum:EtOAc=100:1 to 1:1) to give tert-butyl ((trans)-4-cyanotetrahydrofuran-3-yl)(tosyl)carbamate (22.5 g, 96.1% yield) as a yellow solid. ¹H NMR (400 MHz, DMSO-d₆) δ=7.82 (d, J=8.4 Hz, 2H), 7.47 (d, J=8.0 Hz, 2H), 5.47-5.29 (m, 1H), 4.19-4.15 (m, 1H), 4.09-3.91 (m, 2H), 3.87-3.75 (m, 1H), 3.66 (m, 1H), 2.42 (s, 3H), 1.30 (s, 9H).

### 43.4 Preparation of tert-butyl ((trans)-4-cyanotetrahydrofuran-3-yl)carbamate

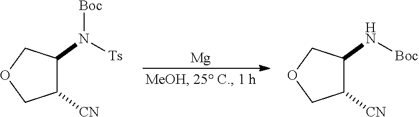

To a solution of tert-butyl ((trans)-4-cyanotetrahydrofuran-3-yl)(tosyl)carbamate (22.5 g, 61.4 mmol, 1 eq) in MeOH (2000 mL) was added magnesium powder (12 g, 491 mmol, 8 eq) in portions carefully at 25° C. Accompanied by generation of heat (exothermic) and bubbling, the resulting suspension was stirred at 25° C. for 1 h. TLC showed the reaction was complete. The reaction was diluted with DCM (400 mL) and poured into cold water (200 mL), then adjust pH=5 with aq. HCl (2N). The organic phase was washed twice with saturated aq. NaHCO₃ (100 mL×2) and brine (100 mL×2), dried with anhydrous Na₂SO₄, filtered, and concentrated in vacuum to give tert-butyl ((trans)-4-cyanotetrahydrofuran-3-yl)carbamate (12 g, crude) as a yellow solid. ¹H NMR (400 MHz, DMSO-d₆) δ=7.61-7.38 (m, 1H), 4.31-4.17 (m, 1H), 4.02-3.91 (m, 2H), 3.91-3.83 (m, 1H), 3.55-3.45 (m, 1H), 3.18 (m, 1H), 1.40 (s, 9H).

### 43.5 Preparation of (trans)-4-aminotetrahydrofuran-3-carbonitrile

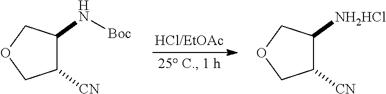

A solution of tert-butyl ((trans)-4-cyanotetrahydrofuran-3-yl)carbamate (12 g, 56.5 mmol, 1 eq) in EtOAc (20 mL) was added HCl/EtOAc (4 M, 120 mL, 8.49 eq) at 25° C., and stirred at 25° C. for 1 hr. TLC showed the reaction was complete. The reaction mixture was concentrated under reduced pressure to give (trans)-4-aminotetrahydrofuran-3-carbonitrile hydrochloride (8 g, 95.2% yield) as a white solid.

### 43.6 Preparation of (trans)-4-((2-chloro-5-methylpyrimidin-4-yl)amino) tetrahydrofuran-3-carbonitrile

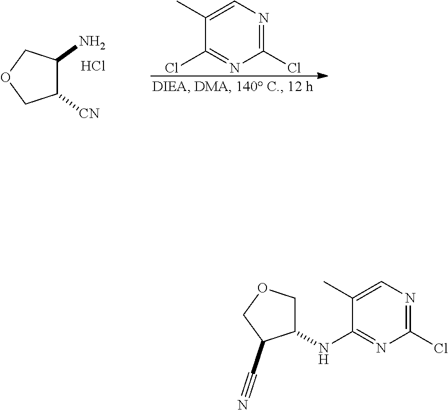

A mixture of 2,4-dichloro-5-methyl-pyrimidine (4.40 g, 26.9 mmol, 1 eq), 4-aminotetrahydrofuran-3-carbonitrile hydrochloride (4 g, 26.9 mmol, 1 eq), DIEA (20.8 g, 161 mmol, 6 eq) in DMA (80 mL) was degassed and purged with N₂ for 3 times, and then the mixture was stirred at 140° C. for 12 hrs under N₂ atmosphere. TLC showed the reaction was complete. The reaction mixture was diluted with H₂O (250 mL) and extracted with EtOAc (50×3 mL). The combined organic layers were washed with brine (100 mL×2), dried over anhydrous Na₂SO₄, filtered, and concentrated under reduced pressure to give a crude product. The crude product was trituration with MTBE (20 mL) to give (trans)-4-((2-chloro-5-methylpyrimidin-4-yl)amino)tetrahydrofuran-3-carbonitrile (8 g, crude) as a yellow solid. ¹H NMR (400 MHz, DMSO-d₆) δ=7.91 (s, 1H), 7.49 (d, J=6.0 Hz, 1H), 4.86-4.77 (m, 1H), 4.16-4.05 (m, 2H), 3.94 (m, 1H), 3.75 (m, 1H), 3.44-3.37 (m, 1H), 3.17 (d, J=5.2 Hz, 1H), 2.01 (s, 3H).

### 43.7 Preparation of (trans)-4-((2-((4-bromo-3-(hydroxymethyl)-5-(trifluoromethyl) phenyl)amino)-5-methylpyrimidin-4-yl)amino)tetrahydrofuran-3-carbonitrile

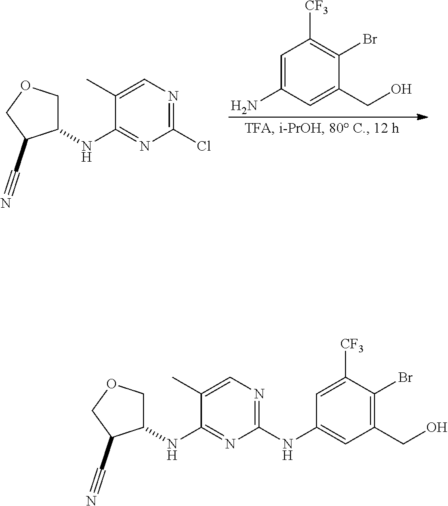 [SMILES: Cc1cnc(Cl)nc1N[C@H]1COC[C@@H]1C#N ; Nc1cc(CO)c(Br)c(C(F)(F)F)c1 ; Cc1cnc(Nc2cc(CO)c(Br)c(C(F)(F)F)c2)nc1N[C@H]1COC[C@@H]1C#N]

A mixture of (trans)-4-((2-chloro-5-methylpyrimidin-4-yl)amino)tetrahydrofuran-3-carbonitrile (2.2 g, 9.2 mmol, 1 eq), [5-amino-2-bromo-3-(trifluoromethyl)phenyl]methanol (2.5 g, 9.2 mmol, 1 eq), TFA (1.6 g, 13.8 mmol, 1.5 eq) in i-PrOH (50 mL) was degassed and purged with N₂ for 3 times, and then the mixture was stirred at 80° C. for 12 hrs under N₂ atmosphere. TLC showed the reaction was complete. The reaction mixture was concentrated under reduced pressure to give a crude product. The crude product was trituration with MTBE (20 mL) to give (trans)-4-[[2-[4-bromo-3-(hydroxymethyl)-5-(trifluoromethyl)anilino]-5-methyl-pyrimidin-4-yl]amino]tetrahydrofuran-3-carbonitrile (4.4 g) as a yellow solid. ¹H NMR (400 MHz, DMSO-d₆) δ=10.90 (s, 1H), 8.57 (s, 1H), 8.09 (d, J=2.0 Hz, 2H), 7.90 (s, 1H), 5.05-5.00 (m, 1H), 4.59 (s, 2H), 4.17 (t, J=8.0 Hz, 1H), 4.15-4.05 (m, 1H), 3.90-3.85 (m, 1H), 3.81-3.62 (m, 2H), 2.06 (s, 3H).

### 43.8 Preparation of (trans)-4-((2-((4-bromo-3-(((tert-butyldimethylsilyl)oxy)methyl)-5-(trifluoromethyl)phenyl)amino)-5-methylpyrimidin-4-yl)amino)tetrahydrofuran-3-carbonitrile

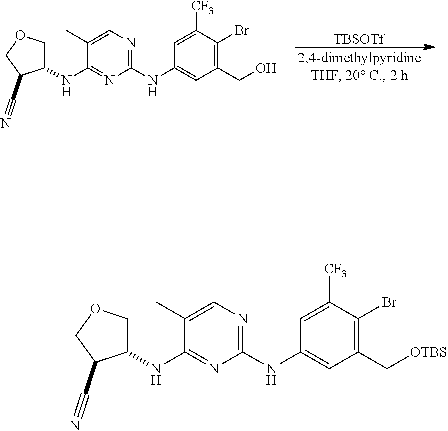 [SMILES: "TBSOTf" ; Cc1cnc(Nc2cc(CO)c(Br)c(C(F)(F)F)c2)nc1N[C@H]1COC[C@@H]1C#N ; Cc1cnc(Nc2cc(CO[Si](C)(C)C(C)(C)C)c(Br)c(C(F)(F)F)c2)nc1N[C@H]1COC[C@@H]1C#N]

A mixture of (trans)-4-[[2-[4-bromo-3-(hydroxymethyl)-5-(trifluoromethyl)anilino]-5-methyl-pyrimidin-4-yl]amino]tetrahydrofuran-3-carbonitrile (4.4 g, 9.3 mmol, 1 eq) in THE (50 mL) was added 2,4-dimethylpyridine (4 g, 37.2 mmol, 4 eq) and TBSOTf (4.9 g, 18.6 mmol, 4.3 mL, 2 eq) at 0° C. degassed and purged with N₂ for 3 times, and then the mixture was stirred at 20° C. for 2 hrs under N₂ atmosphere. TLC showed the reaction was complete. The reaction mixture was diluted with saturated aq. NH₄Cl (50 mL) and extracted with EtOAc (50×3 mL). The combined organic layers were washed with brine (50 mL), dried over anhydrous Na₂SO₄, filtered, and concentrated under reduced pressure to give a residue. The residue was purified by silica gel column chromatography (petroleum ether:EtOAc=100:1 to 1:1) to give (trans)-4-((2-((4-bromo-3-(((tert-butyldimethylsilyl)oxy)methyl)-5-(trifluoromethyl)phenyl)amino)-5-methylpyrimidin-4-yl)amino)tetrahydrofuran-3-carbonitrile (5.5 g, crude) as yellow oil. ¹H NMR (400 MHz, DMSO-d₆) δ=9.54 (s, 1H), 8.39 (d, J=2.4 Hz, 1H), 8.15 (s, 1H), 7.79 (s, 1H), 7.03 (d, J=7.2 Hz, 1H), 5.05-4.95 (m, 1H), 4.73 (s, 2H), 4.20-4.10 (m 2H), 4.00-3.90 (m, 1H), 3.65-3.55 (m, 1H), 3.55-3.50 (m, 1H), 1.97 (s, 3H), 0.95 (s, 9H), 0.14 (s, 6H).

### 43.9 Preparation of (trans)-4-((2-((3-(((tert-butyldimethylsilyl)oxy)methyl)-4-(5,5-dimethyl-1,3,2-dioxaborinan-2-yl)-5-(trifluoromethyl)phenyl)amino)-5-methylpyrimidin-4-yl)amino)tetrahydrofuran-3-carbonitrile

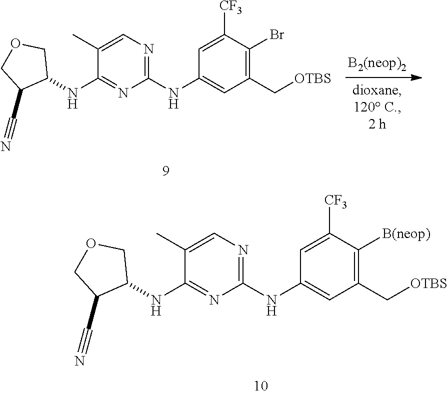

A mixture of (trans)-4-((2-((4-bromo-3-(((tert-butyldimethylsilyl)oxy)methyl)-5-(trifluoromethyl)phenyl)amino)-5-methylpyrimidin-4-yl)amino)tetrahydrofuran-3-carbonitrile (5 g, 8.52 mmol, 1 eq), 2-(5,5-dimethyl-1,3,2-dioxaborinan-2-yl)-5,5-dimethyl-1,3,2-dioxaborinane (13.5 g, 59.6 mmol, 7 eq), KOAc (2.5 g, 25.5 mmol, 3 eq), Pd(PPh₃)₂Cl₂ (598 mg, 852 umol, 0.1 eq) in dioxane (50 mL) was degassed and purged with N₂ for 3 times, and then the mixture was stirred at 120° C. for 2 hrs under N₂ atmosphere. LCMS showed the reaction was complete and desired MS observed. The reaction mixture was concentrated under reduced pressure to give a residue. The residue was purified by prep-HPLC (column: Waters Xbridge Prep OBD C18 150*40 mm*10 um; mobile phase: [water (NH₄HCO₃)-ACN]; B %: 60%-90%, 8 min). to give (trans)-4-((2-((3-(((tert-butyldimethylsilyl)oxy)methyl)-4-(5,5-dimethyl-1,3,2-dioxaborinan-2-yl)-5-(trifluoromethyl)phenyl)amino)-5-methylpyrimidin-4-yl)amino)tetrahydrofuran-3-carbonitrile (3 g, 56.8% yield) as a white solid.

### 43.10 Preparation of (trans)-4-((2-((1-hydroxy-7-(trifluoromethyl)-1,3-dihydrobenzo [c][1,2]oxaborol-5-yl)amino)-5-methylpyrimidin-4-yl)amino)tetrahydrofuran-3-carbonitrile

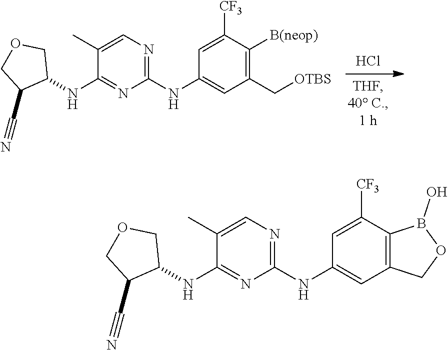

To a solution of (trans)-4-((2-((3-(((tert-butyldimethylsilyl)oxy)methyl)-4-(5,5-dimethyl-1,3,2-dioxaborinan-2-yl)-5-(trifluoromethyl)phenyl)amino)-5-methylpyrimidin-4-yl)amino) tetrahydrofuran-3-carbonitrile (2 g, 3.23 mmol, 1 eq) in THF (20 mL) was added aq. HCl (6 M, 2.15 mL, 4 eq) at 20° C., and then stirred at 40° C. for 1 h. Large amount of solid formed. LCMS showed the reaction was complete and desired MS observed. The reaction mixture was filtered and the filter cake was triturated with THF (10 mL), dried in vacuum to give (trans)-4-((2-((1-hydroxy-7-(trifluoromethyl)-1,3-dihydrobenzo[c][1,2]oxaborol-5-yl)amino)-5-methylpyrimidin-4-yl)amino)tetrahydrofuran-3-carbonitrile (1.4 g, 90% purity) as a white solid. ¹H NMR (400 MHz, DMSO-d₆) δ=9.54 (s, 1H), 8.89 (s, 1H), 8.14 (s, 1H), 8.07 (s, 1H), 7.84 (s, 1H), 7.04 (d, J=7.6 Hz, 1H), 5.13-4.94 (m, 3H), 4.17-4.05 (m, 2H), 4.00-3.95 (m, 1H), 3.70-3.60 (m, 1H), 3.60-3.50 (m, 1H), 1.99 (s, 3H). This material (1 g) was separated by SFC (column: DAICEL CHIRALPAK AD (250 mm*50 mm, 10 um); mobile phase: [0.1% NH₃H₂O IPA]; B %: 30%-30%, 3.3 min) to give (trans)-4-[[2-[[1-hydroxy-7-(trifluoromethyl)-3H-2,1-benzoxaborol-5-yl]amino]-5-methyl-pyrimidin-4-yl]amino]tetrahydrofuran-3-carbonitrile (261.7 mg, 98.52% purity, 95.64% ee, first peak, RT=3.101 min) stereoisomer one as a white solid. ¹H NMR (400 MHz, DMSO-d₆) δ 9.54 (s, 1H), 8.89 (s, 1H), 8.13 (s, 1H), 8.06 (s, 1H), 7.84 (s, 1H), 7.03 (d, J=7.2 Hz, 1H), 5.11-4.96 (m, 3H), 4.17-4.05 (m, 2H), 4.00-3.95 (m, 1H), 3.70-3.60 (m, 1H), 3.57-3.46 (m, 1H), 1.98 (s, 3H) MS (ESI): mass calcd. For C₁₈H₁₇BF₃N₅O₃, 419.14, m/z found 418.0 [M−H]⁻. and (trans)-4-[[2-[[1-hydroxy-7-(trifluoromethyl)-3H-2,1-benzoxaborol-5-yl]amino]-5-methyl-pyrimidin-4-yl]amino]tetrahydrofuran-3-carbonitrile (262.7 mg, 99.1% purity, 99.64% ee, second peak in SFC, RT=3.300 min) stereoisomer two as a white solid. ¹H NMR (400 MHz, DMSO-d₆) δ 9.56 (s, 1H), 8.90 (s, 1H), 8.14 (s, 1H), 8.06 (s, 1H), 7.83 (s, 1H), 7.04 (d, J=7.6 Hz, 1H), 5.18-4.92 (m, 3H), 4.17-4.05 (m, 2H), 4.00-3.95 (m, 1H), 3.70-3.60 (m, 1H), 3.57-3.45 (m, 1H), 1.97 (s, 3H). MS (ESI): mass calcd. For C₁₈H₁₇BF₃N₅O₃, 419.14, m/z found 420.1 [M+H]⁺.

### 44. Preparation of (trans)-3-((2-((1-hydroxy-7-methoxy-1,3-dihydrobenzo[c][1,2]oxaborol-5-yl)amino)-5-methylpyrimidin-4-yl)amino)tetrahydro-2H-pyran-4-carbonitrile

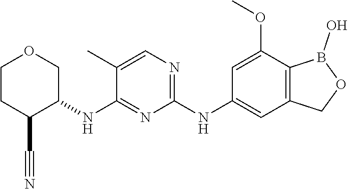 [SMILES: COc1cc(Nc2ncc(C)c(N[C@H]3COCC[C@@H]3C#N)n2)cc2c1B(O)OC2]

### 44.1 Preparation of methyl 2-hydroxy-3-methoxy-5-nitro-benzoate

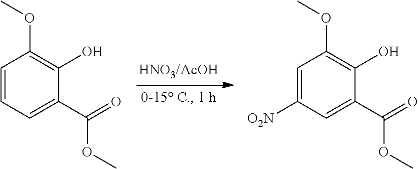 [SMILES: COC(=O)c1cccc(OC)c1O ; COC(=O)c1cc([N+](=O)[O-])cc(OC)c1O]

Two reactions were carried out in parallel. For each a solution of methyl 2-hydroxy-3-methoxy-benzoate (5 g, 27.4 mmol, 1 eq) in AcOH (30 mL) was added HNO₃ (4.04 g, 41.6 mmol, 2.9 mL, 65% purity, 1.52 eq) at 10° C. over 20 min. The mixture was stirred at 15° C. for 40 min. TLC showed the reaction was complete. The two parallel reactions were combined and poured into ice water (90 mL) and then the yellow precipitate formed was collected, washed with H₂O (50 mL×3), Petroleum ether (50 mL×2) to give methyl 2-hydroxy-3-methoxy-5-nitro-benzoate (11.8 g, 94.6% yield) as an off-white solid. ¹H NMR (DMSO-d₆, 400 MHz) δ 11.30 11.20 (m, 1H), 8.19 (s, 1H), 7.90 (s, 1H), 3.95 (s, 3H), 3.92 (s, 3H).

### 44.2 Preparation of methyl 5-amino-2-hydroxy-3-methoxy-benzoate

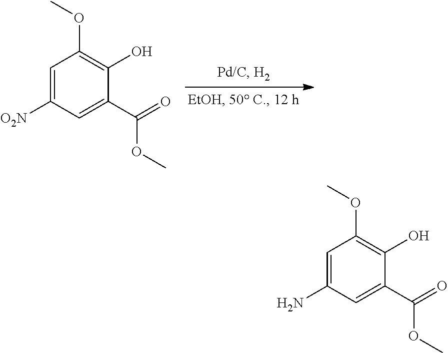 [SMILES: COC(=O)c1cc([N+](=O)[O-])cc(OC)c1O ; COC(=O)c1cc(N)cc(OC)c1O]

To a solution of methyl 2-hydroxy-3-methoxy-5-nitro-benzoate (3.6 g, 15.8 mmol, 1 eq) in EtOH (50 mL) was added Pd/C (1.90 g, 10% purity) at 25° C. The mixture was stirred at 50° C. for 12 h under H₂ (15 Psi). TLC showed the reaction was complete. The reaction mixture was filtered and concentrated under reduced pressure to give methyl 5-amino-2-hydroxy-3-methoxy-benzoate (2.45 g, 78.4% yield) as a yellow solid. ¹H NMR (DMSO-d₆, 400 MHz) δ 9.75 (s, 1H), 6.56 (s, 2H), 4.79 (s, 2H), 3.85 (s, 3H), 3.72 (s, 3H).

### 44.3 Preparation of methyl 5-[[4-[((trans)-4-cyanotetrahydropyran-3-yl)amino]-5-methyl pyrimidin-2-yl]amino]-2-hydroxy-3-methoxy-benzoate

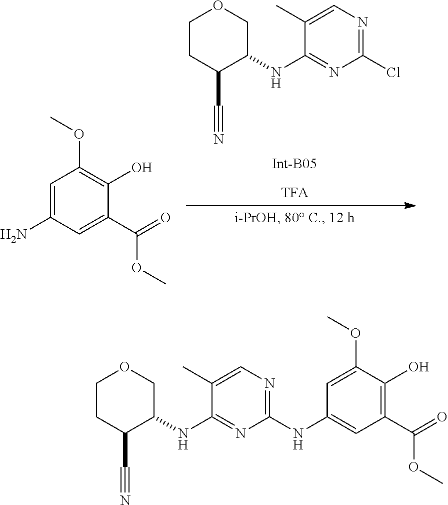 [SMILES: COC(=O)c1cc(N)cc(OC)c1O ; Cc1cnc(Cl)nc1N[C@H]1COCC[C@@H]1C#N ; COC(=O)c1cc(Nc2ncc(C)c(N[C@H]3COCC[C@@H]3C#N)n2)cc(OC)c1O]

To a solution of methyl 5-amino-2-hydroxy-3-methoxy-benzoate (2.51 g, 12.7 mmol, 1 eq) in i-PrOH (30 mL) was added (trans)-3-[(2-chloro-5-methyl-pyrimidin-4-yl)amino]tetrahydropyran-4-carbonitrile (3.22 g, 12.7 mmol, 1 eq) and TFA (2.18 g, 19.1 mmol, 1.41 mL, 1.5 eq) at 25° C. The mixture was stirred at 80° C. for 12 h. Solids precipitated out after 2 h reaction. TLC showed the reaction was complete. The reaction mixture was filtered and then the filter cake was triturated with saturated aq. NaHCO₃ (50 mL) to give methyl 5-[[4-[((trans)-4-cyanotetrahydropyran-3-yl)amino]-5-methyl-pyrimidin-2-yl]amino]-2-hydroxy-3-methoxy-benzoate (4.9 g, 93.1% yield) as a gray solid. ¹H NMR (DMSO-d₆, 400 MHz) δ 10.16 (s, 1H), 8.78 (s, 1H), 7.78-7.70 (m, 2H), 7.53 (s, 1H), 6.59 (d, J=8.8 Hz, 1H), 4.50-4.39 (m, 1H), 3.93-3.82 (m, 5H), 3.78 (s, 3H), 3.31-3.18 (m, 2H), 3.13 (t, J=10.8 Hz, 1H), 2.20-2.11 (m, 1H), 1.93 (s, 3H), 1.86-1.72 (m, 1H).

### 44.4 Preparation of methyl 5-[[4-[((trans)-4-cyanotetrahydropyran-3-yl)amino]-5-methyl pyrimidin-2-yl]amino]-3-methoxy-2-(trifluoromethylsulfonyloxy)benzoate

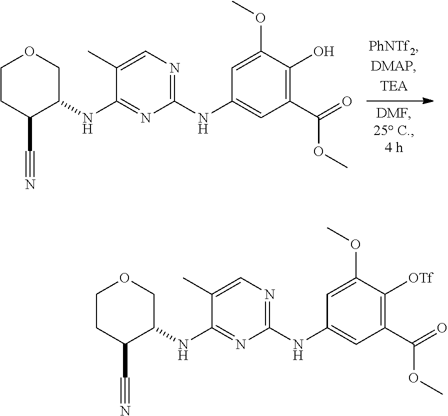 [SMILES: COC(=O)c1cc(Nc2ncc(C)c(N[C@H]3COCC[C@@H]3C#N)n2)cc(OC)c1O ; COC(=O)c1cc(Nc2ncc(C)c(N[C@H]3COCC[C@@H]3C#N)n2)cc(OC)c1OS(=O)(=O)C(F)(F)F]

A mixture of methyl 5-[[4-[((trans)4-cyanotetrahydropyran-3-yl)amino]-5-methyl-pyrimidin-2-yl]amino]-2-hydroxy-3-methoxy-benzoate (2.9 g, 7.01 mmol, 1 eq), 1,1,1-trifluoro-N-phenyl-N-(trifluoromethylsulfonyl)methanesulfonamide (PhNTf₂, 3.76 g, 10.5 mmol, 1.5 eq), DMAP (85.7 mg, 701 umol, 0.1 eq) and TEA (3.55 g, 35.0 mmol, 4.88 mL, 5 eq) in DMF (30 mL) was stirred at 25° C. for 4 h under N₂ atmosphere. TLC showed the reaction was complete. The reaction mixture was quenched by addition of H₂O (30 mL) at 0° C. and extracted with EtOAc (30 mL×3). The combined organic layers were washed with brine (30 mL×2), dried over Na₂SO₄, filtered, and concentrated under reduced pressure to give methyl 5-[[4-[((trans)4-cyanotetrahydropyran-3-yl)amino]-5-methyl-pyrimidin-2-yl]amino]-3-methoxy-2-(trifluoromethylsulfonyloxy)benzoate (3.3 g, 86.2% yield) as a yellow solid. ¹H NMR (DMSO-d₆, 400 MHz) δ 9.51 (s, 1H), 8.01 (d, J=2.4 Hz, 1H), 7.92 (d, J=2.4 Hz, 1H), 7.83 (s, 1H), 6.79 (d, J=8.8 Hz, 1H), 4.54-4.42 (m, 1H), 3.95-3.81 (m, 8H), 3.29-3.18 (m, 2H), 3.12 (t, J=10.8 Hz, 1H), 2.20-2.11 (m, 1H), 1.97 (s, 3H), 1.92-1.78 (m, 1H).

### 44.5 Preparation of methyl 5-[[4-[((trans)-4-cyanotetrahydropyran-3-yl)amino]-5-methylpyrimidin-2-yl]amino]-2-(5,5-dimethyl-1,3,2-dioxaborinan-2-yl)-3-methoxy-benzoate

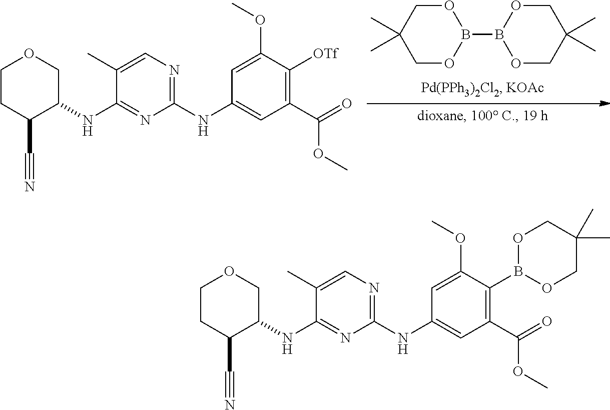

Eight reactions were carried out in parallel. For each a mixture of methyl 5-[[4-[((trans)-4-cyanotetrahydropyran-3-yl)amino]-5-methyl-pyrimidin-2-yl]amino]-3-methoxy-2-(trifluoromethylsulfonyloxy)benzoate (0.5 g, 916 umol, 1 eq), 2-(5,5-dimethyl-1,3,2-dioxaborinan-2-yl)-5,5-dimethyl-1,3,2-dioxaborinane (1.45 g, 6.42 mmol, 7 eq), Pd(PPh₃)₂Cl₂ (96.5 mg, 137 umol, 0.15 eq) and KOAc (197 mg, 2.02 mmol, 2.2 eq) in dioxane (15 mL) was degassed and purged with N₂ for 3 times, and then the mixture was stirred at 100° C. for 9 h under N₂ atmosphere. TLC showed the reaction was complete. The eight parallel reactions were combined, filtered, and concentrated under reduced pressure to give a residue. The residue was purified by column chromatography (SiO₂, petroleum ether/ethyl acetate=1/1 to 0/1) to give methyl 5-[[4-[((trans)-4-cyanotetrahydropyran-3-yl)amino]-5-methyl-pyrimidin-2-yl]amino]-2-(5,5-dimethyl-1,3,2-dioxaborinan-2-yl)-3-methoxy-benzoate (2.5 g, 53.5% yield, 80% purity) as a brown solid. ¹H NMR (DMSO-d₆, 400 MHz) δ 9.19 (s, 1H), 7.90 (s, 1H), 7.79 (s, 1H), 7.62 (s, 1H), 6.69 (d, J=8.8 Hz, 1H), 4.55-4.43 (m, 1H), 3.92-3.82 (m, 5H), 3.74 (s, 3H), 3.64 (s, 4H), 3.29-3.18 (m, 2H), 3.14-3.08 (m, 1H), 2.20-2.11 (m, 1H), 1.95 (s, 3H), 1.88-1.75 (m, 1H), 1.05 (s, 6H).

### 44.6 Preparation of (trans)-3-[[2-[(1-hydroxy-7-methoxy-3H-2,1-benzoxaborol-5-yl)amino]-5-methyl pyrimidin-4-yl]amino]tetrahydropyran-4-carbonitrile

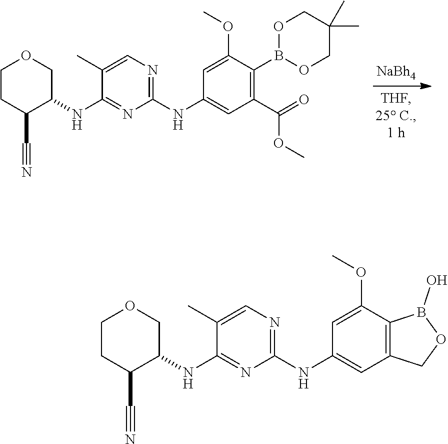

Four reactions were carried out in parallel. For each a solution of methyl 5-[[4-[((trans)-4-cyanotetrahydropyran-3-yl)amino]-5-methyl-pyrimidin-2-yl]amino]-2-(5,5-dimethyl-1,3,2-dioxaborinan-2-yl)-3-methoxy-benzoate (0.5 g, 785 umol, 80% purity, 1 eq) in THE (20 mL) was added NaBH₄ (89.1 mg, 2.36 mmol, 3 eq) at 0° C. After the addition, the reaction was allowed to warm to 25° C. and stirred at 25° C. for 1 h. TLC showed the reaction was complete. The four parallel reactions were combined and quenched by addition of H₂O (20 mL) at 0° C. and adjust pH=6 with aq. HCl (2N), then the precipitate formed was collected to give (trans)-3-[[2-[(1-hydroxy-7-methoxy-3H-2,1-benzoxaborol-5-yl)amino]-5-methyl-pyrimidin-4-yl]amino]tetrahydropyran-4-carbonitrile (300 mg, 24.1% yield, 98.0% purity) as an off-white solid. ¹H NMR (DMSO-d₆, 400 MHz) δ 10.52 (s, 1H), 8.75 (s, 1H), 8.33 (s, 1H), 7.86 (s, 1H), 7.18 (s, 1H), 7.05 (s, 1H), 4.97-4.86 (m, 2H), 4.50-4.38 (m, 1H), 3.95-3.85 (m, 2H), 3.83 (s, 3H), 3.42-3.17 (m, 3H), 2.22-2.14 (m, 1H), 2.05 (s, 3H), 1.88-1.75 (m, 1H). MS (ESI): mass calcd. For C₁₉H₂₂BN₅O₄ 395.18, m/z found 396.2 [M+H]⁺. HPLC: 98.08% (220 nm), 99.12% (254 nm). This material was separated by SFC (column: DAICEL CHIRALPAK AD (250 mm×30 mm, 10 um); mobile phase: [0.1% NH₃H₂O ETOH]; B %: 42%-42%, 9 min) to give (trans)-3-((2-((1-hydroxy-7-methoxy-1,3-dihydrobenzo[c][1,2]oxaborol-5-yl)amino)-5-methylpyrimidin-4-yl)amino)tetrahydro-2H-pyran-4-carbonitrile (71.3 mg, 98.90% purity, 99.26% ee, first peak, RT=4.034 min) stereoisomer one as a white solid. ¹H NMR (DMSO-d₆, 400 MHz) δ 9.17 (s, 1H), 8.48 (s, 1H), 7.79 (s, 1H), 7.46 (s, 1H), 7.24 (s, 1H), 6.71 (d, J=8.8 Hz, 1H), 4.90-4.80 (m, 2H), 4.53-4.41 (m, 1H), 3.93-3.85 (m, 2H), 3.78 (s, 3H), 3.30-3.19 (m, 2H), 3.12 (t, J=10.4 Hz, 1H), 2.21-2.14 (m, 1H), 1.96 (s, 3H), 1.89-1.76 (m, 1H). MS (ESI): mass calcd. For C₁₉H₂₂BN₅O₄ 395.18, m/z found 396.2 [M+H]⁺. HPLC: 98.90% (220 nm), 99.28% (254 nm) and (trans)-3-((2-((1-hydroxy-7-methoxy-1,3-dihydrobenzo[c][1,2]oxaborol-5-yl)amino)-5-methylpyrimidin-4-yl)amino)tetrahydro-2H-pyran-4-carbonitrile (53.4 mg, 99.34% purity, 97.50% ee, second peak, RT=4.255 min) stereoisomer two as a white solid. ¹H NMR (DMSO-d₆, 400 MHz) δ 9.33 (s, 1H), 8.52 (s, 1H), 7.80 (s, 1H), 7.41 (s, 1H), 7.20 (s, 1H), 6.95 (s, 1H), 4.92-4.81 (m, 2H), 4.52-4.42 (m, 1H), 3.93-3.85 (m, 2H), 3.79 (s, 3H), 3.30-3.20 (m, 2H), 3.13 (t, J=10.8 Hz, 1H), 2.21-2.14 (m, 1H), 1.97 (s, 3H), 1.89-1.76 (m, 1H). MS (ESI): mass calcd. For C₁₉H₂₂BN₅O₄ 395.18, m/z found 396.2 [M+H]⁺. HPLC: 99.34% (220 nm), 99.73% (254 nm).

### 45. Preparation of 5-((4-(((trans)-4-cyanotetrahydro-2H-pyran-3-yl)amino)-5-methylpyrimidin-2-yl)amino)-1-hydroxy-1,3-dihydrobenzo[c][1,2]oxaborole-7-carbonitrile

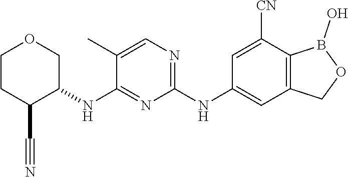

### 45.1 Preparation of 3-((2-((4-bromo-3-cyano-5-(hydroxymethyl)phenyl)amino)-5-methylpyrimidin-4-yl)amino)tetrahydro-2H-pyran-4-carbonitrile

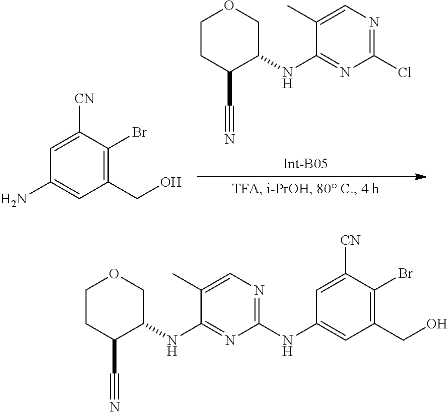

To a solution of 5-amino-2-bromo-3-(hydroxymethyl)benzonitrile (3.8 g, 16.7 mmol, 1 eq) and 3-[(2-chloro-5-methyl-pyrimidin-4-yl)amino]tetrahydropyran-4-carbonitrile (3.8 g, 15 mmol, 0.9 eq) in i-PrOH (40 mL) was added TFA (2.8 g, 25.1 mmol, 1.86 mL, 1.5 eq) at 25° C. The mixture was stirred at 80° C. for 4 hr. LCMS showed the starting material was consumed completely and desired MS was detected. The reaction mixture was quenched with saturated.aq. Na₂CO₃ (50 mL) and extracted with EtOAc (30 mL×3). The combined organic layers were washed with brine (50 mL), dried over anhydrous Na₂SO₄, filtered, and concentrated under reduced pressure to give a crude product. The crude product was triturated with Petroleum ether (30 mL) at 25° C. for 10 min to give (trans)-3-[[2-[4-bromo-3-cyano-5-(hydroxymethyl)anilino]-5-methyl-pyrimidin-4-yl]amino]tetrahydropyran-4-carbonitrile (7.1 g, 95.6% yield) as a brown solid. ¹H NMR (400 MHz, METHANOL-d₄) δ=8.24 (d, J=2.8 Hz, 1H), 8.07 (d, J=2.8 Hz, 1H), 7.77 (s, 1H), 4.67 (s, 2H), 4.01-3.97 (m, 1H), 3.95 (t, J=3.2 Hz, 1H), 3.80-3.70 (m, 1H), 3.50-3.40 (m, 1H), 3.27-3.22 (m, 1H), 3.20-3.10 (m, 1H), 2.17-2.13 (m, 1H), 2.03 (s, 3H), 1.90-1.80 (m, 1H).

### 45.2 Preparation of (trans)-3-((2-((4-bromo-3-(((tert-butyldimethylsilyl)oxy)methyl)-5-cyanophenyl)amino)-5-methylpyrimidin-4-yl)amino)tetrahydro-2H-pyran-4-carbonitrile

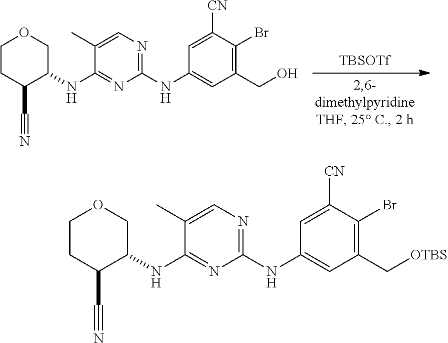

To a solution of (trans)-3-[[2-[4-bromo-3-cyano-5-(hydroxymethyl)anilino]-5-methyl-pyrimidin-4-yl]amino]tetrahydropyran-4-carbonitrile (3.5 g, 7.90 mmol, 1 eq) in THF (40 mL) was added 2,6-dimethylpyridine (3.38 g, 31.5 mmol, 3.68 mL, 4 eq) and [tert-butyl(dimethyl)silyl] trifluoromethanesulfonate (4.17 g, 15.8 mmol, 3.63 mL, 2 eq) at 25° C. The mixture was stirred at 25° C. for 2 hr. LCMS showed the starting material was consumed completely and desired MS was detected. The reaction mixture was concentrated under reduced pressure to give a crude product. The crude product was triturated with petroleum ether (20 ml) at 25° C. for 15 min to give 3-[[2-[4-bromo-3-[[tert-butyl(dimethyl)silyl]oxymethyl]-5-cyano-anilino]-5-methyl-pyrimidin-4-yl]amino]tetrahydropyran-4-carbonitrile (2.3 g, 52.2% yield) as a brown solid. ¹H NMR (400 MHz, CDCl₃) δ=8.26 (d, J=2.4 Hz, 1H), 7.84 (s, 1H), 7.78 (d, J=2.4 Hz, 1H), 7.00 (s, 1H), 5.03 (d, J=7.6 Hz, 1H), 4.72 (s, 2H), 4.45-4.35 (m, 1H), 4.17-4.09 (m, 1H), 3.95-3.82 (m, 2H), 3.80-3.70 (m, 1H), 3.30-3.25 (m, 1H), 2.20-2.10 (m, 1H), 2.04 (s, 3H), 1.92-1.83 (m, 1H), 0.99 (s, 9H), 0.17 (s, 6H).

### 45.3 Preparation of (2-(((tert-butyldimethylsilyl)oxy)methyl)-6-cyano-4-((4-(((trans)-4-cyanotetrahydro-2H-pyran-3-yl)amino)-5-methylpyrimidin-2-yl)amino)phenyl)boronic acid

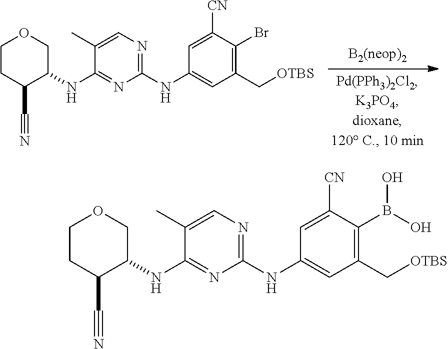

Four reaction were carried out in parallel. For each a solution of (trans)-3-[[2-[4-bromo-3-[[tert-butyl(dimethyl)silyl]oxymethyl]-5-cyano-anilino]-5-methyl-pyrimidin-4-yl]amino]tetrahydropyran-4-carbonitrile (500 mg, 897 umol, 1 eq) and 2-(5,5-dimethyl-1,3,2-dioxaborinan-2-yl)-5,5-dimethyl-1,3,2-dioxaborinane (810 mg, 3.59 mmol, 4 eq) in dioxane (5 mL) was added Pd(PPh₃)₂Cl₂ (62.9 mg, 89.6 umol, 0.1 eq) and K₃PO₄ (571 mg, 2.69 mmol, 3 eq). The mixture was stirred at 120° C. for 10 min. LCMS showed the starting material was consumed completely and desired MS was detected. The four parallel reactions were combined and concentrated under reduced pressure to give a residue. The residue was purified by prep-HPLC (column: C18 (250*50 mm*10 um); mobile phase: [water (NH₄HCO₃)-ACN]; B %: 35%-65%, 10 min) to give (2-(((tert-butyldimethylsilyl)oxy)methyl)-6-cyano-4-((4-(((trans)-4-cyanotetrahydro-2H-pyran-3-yl)amino)-5-methylpyrimidin-2-yl)amino)phenyl)boronic acid (0.8 g, 42.6% yield) as a white solid.

### 45.4 Preparation of 5-((4-(((trans)4-cyanotetrahydro-2H-pyran-3-yl)amino)-5-methylpyrimidin-2-yl)amino)-1-hydroxy-1,3-dihydrobenzo[c][1,2]oxaborole-7-carbonitrile

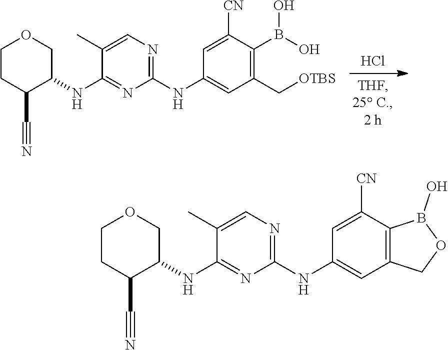

To a solution of (2-(((tert-butyldimethylsilyl)oxy)methyl)-6-cyano-4-((4-(((trans)-4-cyanotetrahydro-2H-pyran-3-yl)amino)-5-methylpyrimidin-2-yl)amino)phenyl)boronic acid (400 mg, 765 umol, 1 eq) in THF (4 mL) was added aq. HCl (6 M, 5 mL). The mixture was stirred at 25° C. for 2 hr. LCMS showed the starting material was consumed completely and desired MS was detected. The reaction mixture was diluted with H₂O (10 mL) and extracted with EtOAc (15 mL×3). The combined organic layers were washed with brine (10 mL×2), dried over anhydrous Na₂SO₄, filtered, and concentrated under reduced pressure to give a residue. The residue was purified by prep-HPLC (column: Phenomenex Luna C18 75*30 mm*3 um; mobile phase: [water (formic acid-FA)-ACN]; B %: 1%-35%, 8 min) to give 5-[[4-[(4-cyanotetrahydropyran-3-yl)amino]-5-methyl-pyrimidin-2-yl]amino]-1-hydroxy-3H-2,1-benzoxaborole-7-carbonitrile (140 mg, 100% purity) as a white solid. ¹H NMR (400 MHz, DMSO-d₆) δ=9.59 (s, 1H), 9.17 (s, 1H), 8.15 (s, 1H), 8.14 (s, 1H), 8.12 (s, 1H), 7.84 (s, 1H), 6.82 (d, J=8.8 Hz, 1H), 5.05-4.94 (m, 2H), 4.53-4.42 (m, 1H), 3.92-3.85 (m, 2H), 3.20-3.15 (m, 2H), 3.15-3.10 (m, 1H), 2.20-2.15 (m, 1H), 1.97 (s, 3H), 1.94-1.84 (m, 1H). MS (ESI): mass calcd. For C₁₉H₁₉BN₆O₃ 390.16, m/z found 389.0 [M−H]⁻. HPLC: 100.00% (220 nm), 100.00% (254 nm).

### 46. Preparation of (trans)-3-((2-((7-chloro-1-hydroxy-1,3-dihydrobenzo[c][1,2]oxaborol-5-yl)amino)-5 fluoropyrimidin-4-yl)amino)tetrahydro-2H-pyran-4-carbonitrile

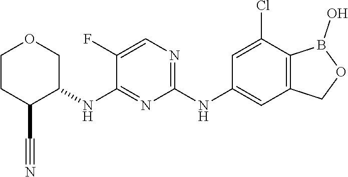

### 46.1 Preparation of (trans)-3-((2-((7-chloro-1-hydroxy-1,3-dihydrobenzo[c][1,2]oxaborol-5-yl)amino)-5 fluoropyrimidin-4-yl)amino)tetrahydro-2H-pyran-4-carbonitrile

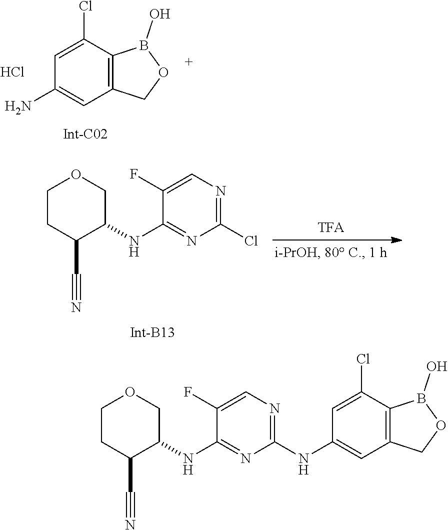

To a solution of 7-chloro-1-hydroxy-3H-2,1-benzoxaborol-5-amine (250 mg, 1.14 mmol, 1 eq, HCl salt) in i-PrOH (2 mL) was added (trans)-3-((2-chloro-5-fluoropyrimidin-4-yl)amino) tetrahydro-2H-pyran-4-carbonitrile (205 mg, 798 umol, 0.7 eq), TFA (195 mg, 1.70 mmol, 1.5 eq) at 25° C., and then the mixture was stirred at 80° C. for 1 h. LCMS showed the reaction was complete and desired MS observed. The reaction mixture was quenched by addition H₂O (0.5 mL) at 25° C., filtered, and concentrated under reduced pressure to give a residue. The residue was purified by prep-HPLC (column: Waters Xbridge BEH C₁₈ 100×30 mm×10 um; mobile phase: [water (NH₄HCO₃)-ACN]; B %: 10%-40%, 8 min) to give (trans)-3-((2-((7-chloro-1-hydroxy-1,3-dihydrobenzo[c][1,2]oxaborol-5-yl)amino)-5-fluoropyrimidin-4-yl)amino) tetrahydro-2H-pyran-4-carbonitrile (7.3 mg, 97.48% purity) as a white solid. ¹H NMR (DMSO-d₆, 400 MHz) δ=9.62 (s, 1H), 8.05 (s, 1H), 7.80-7.78 (m, 1H), 7.75 (d, J=5.6 Hz, 2H), 6.05 (s, 1H), 4.93 (s, 2H), 4.41-4.32 (m, 1H), 3.92-3.86 (m, 2H), 3.11 (m, 3H), 2.20-2.16 (m, 1H), 1.88-1.84 (m, 1H). MS (ESI): mass calcd. For C₁₇H₁₆BClFN₅O₃ 403.10, m/z found 402.0 [M−H]⁻. HPLC: 97.48% (220 nm), 98.66% (254 nm).

### 47. Preparation of (trans)-3-((5-chloro-2-((1-hydroxy-1,3-dihydrobenzo[c][1,2]oxaborol-5-yl)amino)pyrimidin-4-yl)amino)tetrahydro-2H-pyran-4-carbonitrile

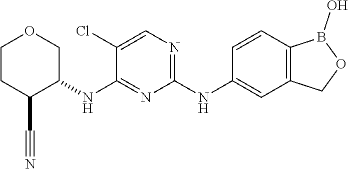

### 47.1 Preparation of methyl 2-bromo-5-((tert-butoxycarbonyl)amino)benzoate

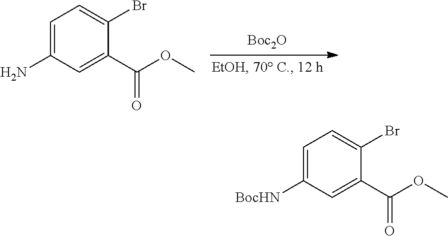 [SMILES: "Boc2O" ; COC(=O)c1cc(N)ccc1Br ; COC(=O)c1cc(NC(=O)OC(C)(C)C)ccc1Br]

To a mixture of methyl 5-amino-2-bromobenzoate (22 g, 95.6 mmol, 1 eq) in EtOH (200 mL) was added Boc₂O (31.3 g, 143 mmol, 1.5 eq) and stirred at 70° C. for 12 hrs. TLC showed the reaction was complete. The reaction mixture was concentrated under reduced pressure to give a residue. The residue was triturated with petroleum ether (100 mL) at 20° C. for 2 hrs to give methyl 2-bromo-5-((tert-butoxycarbonyl)amino)benzoate (30 g, 95.0% yield) as a pink solid. ¹H NMR (CDCl₃, 400 MHz) δ 7.83 (s, 1H), 7.56 (d, J=8.8 Hz, 1H), 7.40 (d, J=8.8 Hz, 1H), 6.55 (s, 1H), 3.93 (s, 3H), 1.52 (s, 9H).

### 47.2 Preparation of methyl 5-((tert-butoxycarbonyl)amino)-2-(5,5-dimethyl-1,3,2-dioxaborinan-2-yl)benzoate

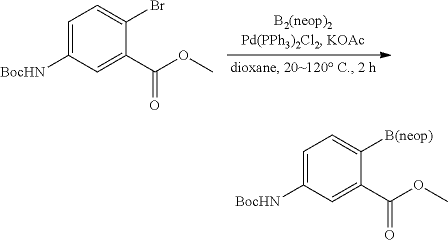

To a mixture of methyl 2-bromo-5-((tert-butoxycarbonyl)amino)benzoate (500 mg, 3.02 mmol, 1 eq) in dioxane (20 mL) was added 5,5,5′,5′-tetramethyl-2,2′-bi(1,3,2-dioxaborinane) (3.42 g, 15.1 mmol, 5 eq), KOAc (890 mg, 9.08 mmol, 3 eq) and Pd(PPh₃)₂Cl₂ (212 mg, 302 umol, 0.1 eq) at 20° C. and stirred at 120° C. for 2 hrs under N₂. LCMS showed the reaction was complete and desired MS observed. The reaction mixture was filtered and concentrated under reduced pressure to give methyl 5-((tert-butoxycarbonyl)amino)-2-(5,5-dimethyl-1,3,2-dioxaborinan-2-yl)benzoate (1 g, crude) as brown oil.

### 47.3 Preparation of tert-butyl (1-hydroxy-1,3-dihydrobenzo[c][1,2]oxaborol-5-yl)carbamate

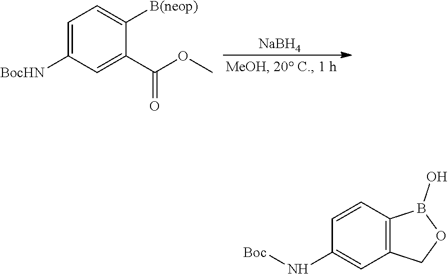

To a mixture of methyl 5-((tert-butoxycarbonyl)amino)-2-(5,5-dimethyl-1,3,2-dioxaborinan-2-yl)benzoate (1 g, 2.76 mmol, 1 eq, crude) in MeOH (10 mL) was added NaBH₄ (518 mg, 8.28 mmol, 3 eq) at 20° C., and then the mixture was stirred at 20° C. for 1 hr. LCMS showed the reaction was complete and desired MS was detected. The reaction mixture was quenched by addition aq. HCl (2N, 20 mL) at 0° C., and then extracted with EtOAc (30 mL×3). The combined organic layers were washed with brine (30 mL×2), dried over Na₂SO₄, filtered, and concentrated under reduced pressure to give a residue. The residue was purified by flash silica gel chromatography (ISCO®; 12 g SepaFlash® Silica Flash Column, Eluent of 0˜50% Ethyl acetate/petroleum ether gradient @ 36 mL/min) to give tert-butyl (1-hydroxy-1,3-dihydrobenzo[c][1,2]oxaborol-5-yl)carbamate (600 mg, crude) as yellow oil.

### 47.4 Preparation of 5-aminobenzo[c][1,2]oxaborol-1(3H)-ol (hydrogen chloride)

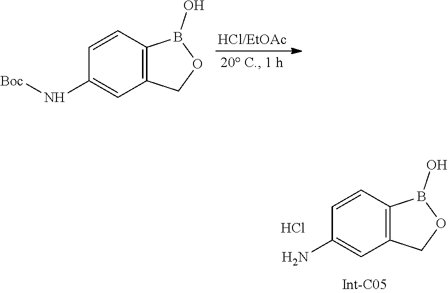 [SMILES: CC(C)(C)OC(=O)Nc1ccc2c(c1)COB2O ; Cl ; Nc1ccc2c(c1)COB2O]

A mixture of tert-butyl (1-hydroxy-1,3-dihydrobenzo[c][1,2]oxaborol-5-yl)carbamate (600 mg, 2.41 mmol, 1 eq) in HCl/EtOAc (10 mL, 4 M) was stirred at 20° C. for 1 hr. LCMS showed the reaction was complete and desired MS was detected. The reaction mixture was concentrated under reduced pressure to give 5-aminobenzo[c][1,2]oxaborol-1(3H)-ol (370 mg, 82.8% yield, HCl) as a yellow solid. ¹H NMR (DMSO-d₆, 400 MHz) δ 9.29 (s, 3H), 7.73 (d, J=8.0 Hz, 1H), 7.21 (s, 1H), 7.16 (d, J=8.0 Hz, 1H), 4.97 (s, 2H).

### 47.5 Preparation of (trans)-3-((5-chloro-2-((1-hydroxy-1,3-dihydrobenzo[c][1,2]oxaborol-5-yl)amino)pyrimidin-4-yl)amino)tetrahydro-2H-pyran-4-carbonitrile

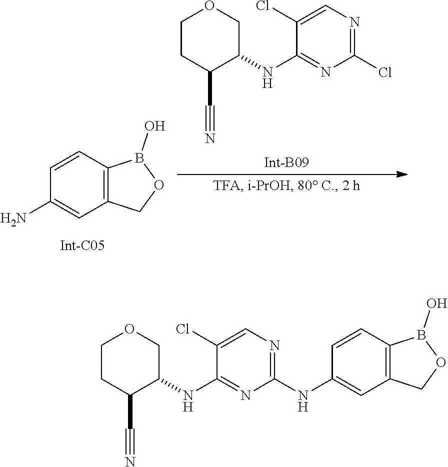 [SMILES: Nc1ccc2c(c1)COB2O ; N#C[C@H]1CCOC[C@@H]1Nc1nc(Cl)ncc1Cl ; N#C[C@H]1CCOC[C@@H]1Nc1nc(Nc2ccc3c(c2)COB3O)ncc1Cl]

To a mixture of (trans)-3-((2,5-dichloropyrimidin-4-yl)amino)tetrahydro-2H-pyran-4-carbonitrile (206 mg, 756 umol, 0.7 eq) in i-PrOH (5 mL) was added 5-aminobenzo[c][1,2]oxaborol-1(3H)-ol (200 mg, 1.08 mmol, 1 eq, HCl) and TFA (184 mg, 1.62 mmol, 119 uL, 1.5 eq), and stirred at 80° C. for 2 hrs under N₂. LCMS showed the reaction was complete and desired MS was detected. The reaction mixture was quenched by addition H₂O (5 mL), filtered to obtain the filter cake as a crude product. The crude product was purified by prep-HPLC (column: Phenomenex luna C₁₈ 80*40 mm*3 um; mobile phase: [water (HCl)-ACN]; B %: 5%-30%, 7 min) to give (trans)-3-((5-chloro-24(1-hydroxy-1,3-dihydrobenzo[c][1,2]oxaborol-5-yl)amino)pyrimidin-4-yl)amino)tetrahydro-2H-pyran-4-carbonitrile (27.9 mg, 6.37% yield, 95.03% purity) as a white solid. ¹H NMR (DMSO-d₆, 400 MHz) δ 9.90 (s, 1H), 8.15 (s, 1H), 7.86 (s, 1H), 7.72 (d, J=7.6 Hz, 1H), 7.63 (d, J=8.0 Hz, 1H), 7.51 (d, J=8.0 Hz, 1H), 5.01-4.89 (m, 2H), 4.47-4.36 (m, 1H), 3.90-3.86 (m, 2H), 3.43-3.17 (m, 3H), 2.19-2.16 (m, 1H), 1.89-1.80 (m, 1H). MS (ESI): mass calcd. For C₁₇H₁₇BClN₅O₃ 385.11, m/z found 383.9 [M−H]⁻. HPLC: 95.03% (220 nm), 99.70% (254 nm).

### 48. Preparation of (trans)-3-[[5-chloro-2-[(7-chloro-1-hydroxy-3H-2,1-benzoxaborol-5-yl)amino]pyrimidin-4-yl]amino]tetrahydropyran-4-carbonitrile

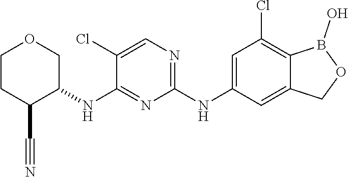

### 48.1 Preparation of (trans)-3-((2-((4-bromo-3-chloro-5-(hydroxymethyl) phenyl)amino)-5-chloropyrimidin-4-yl)amino)tetrahydro-2H-pyran-4-carbonitrile

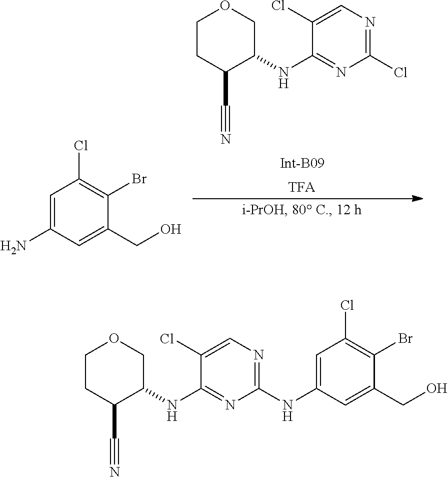 [SMILES: N#C[C@H]1CCOC[C@@H]1Nc1nc(Cl)ncc1Cl ; Nc1cc(Cl)c(Br)c(CO)c1 ; N#C[C@H]1CCOC[C@@H]1Nc1nc(Nc2cc(Cl)c(Br)c(CO)c2)ncc1Cl]

To a solution of (5-amino-2-bromo-3-chloro-phenyl)methanol (500 mg, 2.11 mmol, 1 eq) and (trans)-3-[(2,5-dichloropyrimidin-4-yl)amino]tetrahydropyran-4-carbonitrile (577 mg, 2.11 mmol, 1 eq) in i-PrOH (10 mL) was drop-wise added TFA (361 mg, 3.17 mmol, 1.5 eq) at 25° C. The resulting mixture was stirred at 80° C. for 12 h. Solid was precipitate out. TLC showed the reaction was complete. The reaction mixture was filtered to give a residue. The residue was triturated with saturated aq. NaHCO₃ (10 mL) and filtered to give 3-((2-((4-bromo-3-chloro-5-(hydroxymethyl)phenyl)amino)-5-chloropyrimidin-4-yl)amino)tetrahydro-2H-pyran-4-carbonitrile (0.5 g, 50% yield) as a white solid. ¹H NMR (DMSO-d₆, 400 MHz) δ 10.04 (s, 1H), 8.17 (s, 1H), 8.06 (s, 1H), 7.78-7.71 (m, 2H), 4.50 (s, 2H), 4.49-4.45 (m, 1H), 3.93-3.80 (m, 2H), 3.38-3.28 (m, 1H), 3.25-3.19 (m, 2H), 2.21-2.15 (m, 1H), 2.10-1.85 (m, 1H).

### 48.2 Preparation of (trans)-3-[[2-[4-bromo-3-[[tert-butyl(dimethyl)silyl]oxymethyl]-5-chloro-anilino]-5-chloro pyrimidin-4-yl]amino]tetrahydropyran-4-carbonitrile

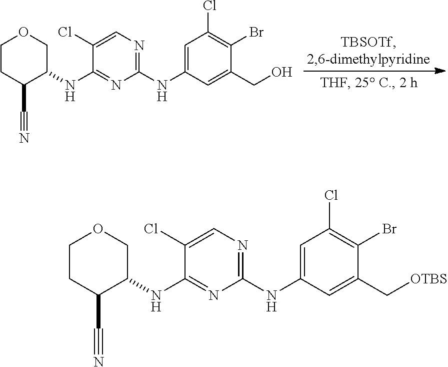 [SMILES: N#C[C@H]1CCOC[C@@H]1Nc1nc(Nc2cc(Cl)c(Br)c(CO)c2)ncc1Cl ; CC(C)(C)[Si](C)(C)OCc1cc(Nc2ncc(Cl)c(N[C@H]3COCC[C@@H]3C#N)n2)cc(Cl)c1Br]

To a solution of (trans)-3-((2-((4-bromo-3-chloro-5-(hydroxymethyl)phenyl)amino)-5-chloropyrimidin-4-yl)amino)tetrahydro-2H-pyran-4-carbonitrile (0.5 g, 1.06 mmol, 1 eq) in THF (5 mL) was added 2,6-dimethylpyridine (452 mg, 4.23 mmol, 490 uL, 4 eq) and [tert-butyl(dimethyl)silyl] trifluoromethanesulfonate (419 mg, 1.59 mmol, 364 uL, 1.5 eq) at 25° C. The mixture was stirred at 25° C. for 2 h. TLC showed the reaction was complete. The reaction mixture was quenched by addition of H₂O (20 mL) at 0° C. and extracted with EtOAc (30 mL×3). The combined organic layers were washed with brine (20 mL×2), dried over Na₂SO₄, filtered, and concentrated under reduced pressure to give a residue. The residue was purified by column chromatography (SiO₂, petroleum ether/ethyl acetate=10/1 to 5/1) to give (trans)-3-[[2-[4-bromo-3-[[tert-butyl(dimethyl) silyl]oxymethyl]-5-chloro-anilino]-5-chloro-pyrimidin-4-yl]amino]tetrahydropyran-4-carbonitrile (0.6 g, 96.6% yield) as a white solid.

### 48.3 Preparation of (trans)-3-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino)tetrahydro-2H-pyran-4-carbonitrile

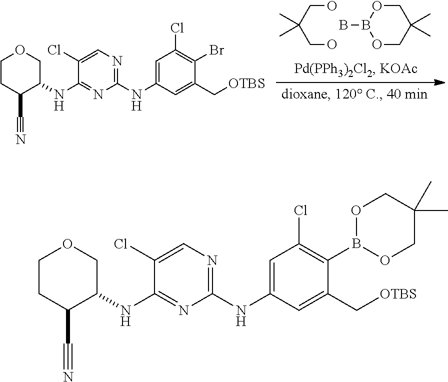

Two reactions were carried out in parallel. For each mixture of (trans)-3-[[2-[4-bromo-3-[[tert-butyl(dimethyl) silyl]oxymethyl]-5-chloro-anilino]-5-chloro-pyrimidin-4-yl]amino]tetrahydropyran-4-carbonitrile (250 mg, 425 umol, 1 eq), 2-(5,5-dimethyl-1,3,2-dioxaborinan-2-yl)-5,5-dimethyl-1,3,2-dioxaborinane (480 mg, 2.13 mmol, 5 eq), KOAc (125 mg, 1.28 mmol, 3 eq) and Pd(PPh₃)₂Cl₂ (30 mg, 42.5 umol, 0.1 eq) in dioxane (5 mL) was degassed and purged with N₂ for 3 times, and then the mixture was stirred at 120° C. for 40 min under N₂ atmosphere. TLC showed the reaction was complete. The two parallel reactions were combined, filtered and the filtrate was concentrated under reduced pressure to give a residue. The residue was purified by column chromatography (SiO₂, petroleum ether/ethyl acetate=3/1 to 1/1) to give (trans)-3-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino)tetrahydro-2H-pyran-4-carbonitrile (0.5 g, crude) as a white solid.

### 48.4 Preparation of (trans)-3-[[5-chloro-2-[(7-chloro-1-hydroxy-3H-2,1-benzoxaborol-5-yl)amino]pyrimidin-4-yl]amino]tetrahydropyran-4-carbonitrile

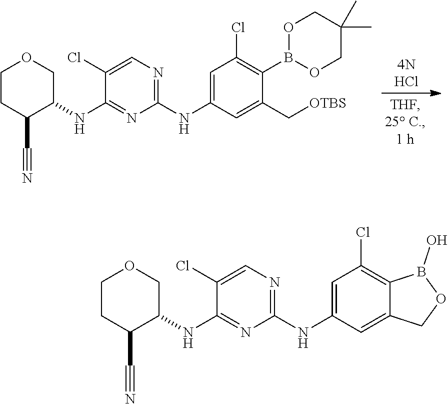

To a solution of (trans)-3-((2-((3-(((tert-butyldimethylsilyl)oxy)methyl)-5-chloro-4-(5,5-dimethyl-1,3,2-dioxaborinan-2-yl)phenyl)amino)-5-chloropyrimidin-4-yl)amino)tetrahydro-2H-pyran-4-carbonitrile (0.5 g, 806 umol, 1 eq) in THE (10 mL) was added aq. HCl (4 M, 2.0 mL, 10 eq) at 25° C. The mixture was stirred at 25° C. for 1 h. Solid was precipitate out. The reaction mixture was filtered to give a residue. The residue was triturated with MeCN (8 mL) and filtered to give (trans)-3-[[5-chloro-2-[(7-chloro-1-hydroxy-3H-2,1-benzoxaborol-5-yl)amino]pyrimidin-4-yl]amino]tetrahydropyran-4-carbonitrile (80.2 mg, 23.7% yield, 98.31% purity) as a white solid. ¹H NMR (DMSO-d₆, 400 MHz) δ 9.82 (s, 1H), 8.13 (s, 1H), 7.74 (d, J=6.8 Hz, 2H), 7.49 (d, J=8.8 Hz, 1H), 5.00-4.90 (m, 2H), 4.52-4.42 (m, 1H), 3.93-3.80 (m, 2H), 3.38-3.28 (m, 2H), 3.25-3.15 (m, 1H), 2.21-2.15 (m, 1H), 2.10-1.85 (m, 1H). MS (ESI): mass calcd. For C₁₇H₁₆BCl₂N₅O₃ 419.07, m/z found 420.1 [M+H]⁺. HPLC: 98.31% (220 nm), 99.12% (254 nm).

### 49. Preparation of (trans)-3-((2-((7-chloro-1-hydroxy-1,3-dihydrobenzo[c][1,2]oxaborol-5-yl)amino)-5-(trifluoromethyl)pyrimidin-4-yl)amino)tetrahydro-2H-pyran-4-carbonitrile

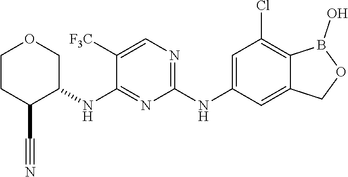

### 49.1 Preparation of (trans)-3-((2-((4-bromo-3-chloro-5-(hydroxymethyl)phenyl) amino)-5-(trifluoromethyl)pyrimidin-4-yl)amino)tetrahydro-2H-pyran-4-carbonitrile

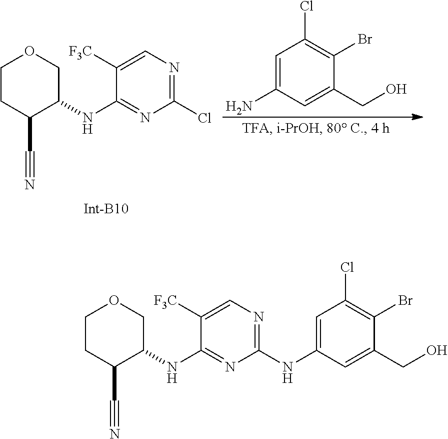

To a solution of (5-amino-2-bromo-3-chloro-phenyl)methanol (500 mg, 2.11 mmol, 1 eq) in i-PrOH (5 mL) was added TFA (361 mg, 3.17 mmol, 1.5 eq) and (trans)-3-[[2-chloro-5-(trifluoromethyl)pyrimidin-4-yl]amino]tetrahydropyran-4-carbonitrile (647 mg, 2.11 mmol, 1 eq) at 25° C. The reaction mixture was stirred at 80° C. for 4 hrs. Solids precipitated out. TLC showed the reaction was complete. The reaction mixture was filtered and the filter cake was triturated with saturated aq. NaHCO₃ (30 mL) and filtered to give (trans)-3-((2-((4-bromo-3-chloro-5-(hydroxymethyl)phenyl)amino)-5-(trifluoromethyl)pyrimidin-4-yl)amino)tetrahydro-2H-pyran-4-carbonitrile (670 mg, 62.7% yield) as a white solid. ¹H NMR (400 MHz, DMSO-d₆) δ=10.12 (s, 1H), 8.34 (s, 1H), 8.09 (s, 1H), 7.79 (d, J=2.8 Hz, 1H), 7.20 (d, J=8.8 Hz, 1H), 4.72-4.58 (m, 1H), 4.55-4.47 (m, 2H), 3.92-3.76 (m, 2H), 3.40-3.35 (m, 1H), 3.31-3.18 (m, 2H), 2.21-2.08 (m, 1H), 1.98-1.79 (m, 1H).

### 49.2 Preparation of (trans)-3-((2-((4-bromo-3-(((tert-butyldimethylsilyl)oxy)methyl)-5-chlorophenyl)amino)-5-(trifluoromethyl)pyrimidin-4-yl)amino)tetrahydro-2H-pyran-4-carbonitrile

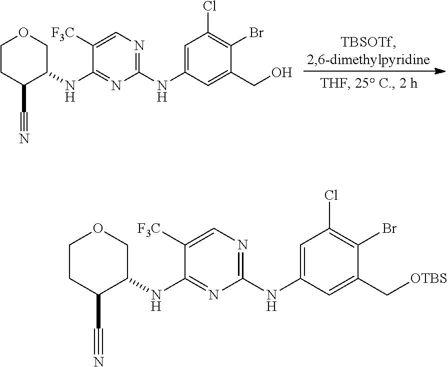 [SMILES: N#C[C@H]1CCOC[C@@H]1Nc1nc(Nc2cc(Cl)c(Br)c(CO)c2)ncc1C(F)(F)F ; CC(C)(C)[Si](C)(C)OCc1cc(Nc2ncc(C(F)(F)F)c(N[C@H]3COCC[C@@H]3C#N)n2)cc(Cl)c1Br]

To a solution of (trans)-3-((2-((4-bromo-3-chloro-5-(hydroxymethyl)phenyl)amino)-5-(trifluoromethyl)pyrimidin-4-yl)amino)tetrahydro-2H-pyran-4-carbonitrile (500 mg, 986 umol, 1 eq) in THF (20 mL) was added 2,6-dimethylpyridine (422 mg, 3.95 mmol, 4 eq) and [tert-butyl(dimethyl)silyl] trifluoromethanesulfonate (391 mg, 1.48 mmol, 1.5 eq) at 25° C. The mixture was stirred at 25° C. for 2 hrs. TLC showed the reaction was complete. The reaction mixture was quenched by addition of H₂O (20 mL) at 0° C. and extracted with EtOAc (10 mL×3). The combined organic layers were washed with brine (20 mL×2), dried over Na₂SO₄, filtered, and concentrated under reduced pressure to give a residue. The residue was purified by silica gel column chromatography (Petroleum ether/ethyl acetate=5/1 to 3/1) to give 3-((2-((4-bromo-3-(((tert-butyldimethylsilyl)oxy)methyl)-5-chlorophenyl)amino)-5-(trifluoromethyl)pyrimidin-4-yl)amino)tetrahydro-2H-pyran-4-carbonitrile (320 mg, 52.2% yield) as a white solid. ¹H NMR (400 MHz, DMSO-d₆) δ=10.16 (s, 1H), 8.30 (s, 1H), 8.20 (s, 1H), 7.72 (s, 1H), 7.16 (d, J=8.8 Hz, 1H), 4.76-4.53 (m, 3H), 3.90-3.76 (m, 2H), 3.53-3.36 (m, 1H), 3.28-3.19 (m, 2H), 2.16 (d, J=11.2 Hz, 1H), 1.90-1.80 (m, 1H), 0.94 (s, 9H), 0.13 (s, 6H).

### 49.3 Preparation of (trans)-3-((2-((3-(((tert-butyldimethylsilyl)oxy)methyl)-5-chloro-4-(5,5-dimethyl-1,3,2-dioxaborinan-2-yl)phenyl)amino)-5-(trifluoromethyl)pyrimidin-4-yl)amino)tetrahydro-2H-pyran-4-carbonitrile

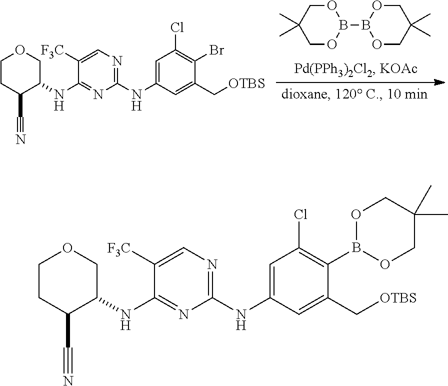

Two reactions were carried out in parallel. For each a mixture of (trans)-3-((2-((4-bromo-3-(((tert-butyldimethylsilyl)oxy)methyl)-5-chlorophenyl)amino)-5-(trifluoromethyl)pyrimidin-4-yl)amino)tetrahydro-2H-pyran-4-carbonitrile (277 mg, 446 umol, 1 eq), 2-(5,5-dimethyl-1,3,2-dioxaborinan-2-yl)-5,5-dimethyl-1,3,2-dioxaborinane (503 mg, 2.23 mmol, 5 eq), Pd(PPh₃)₂Cl₂ (31.3 mg, 44.6 umol, 0.1 eq) and KOAc (131 mg, 1.34 mmol, 3 eq) in dioxane (8 mL) was degassed and purged with N₂ for 3 times, and then the mixture was stirred at 120° C. for 10 min under N₂ atmosphere. TLC showed the reaction was complete. The parallel reactions were combined, filtered and the filtrate was concentrated under reduced pressure to give a residue. The residue was purified by column chromatography (SiO₂, Petroleum ether/ethyl acetate=3/1 to 1/1) to give (trans)-3-((2-((3-(((tert-butyldimethylsilyl)oxy)methyl)-5-chloro-4-(5,5-dimethyl-1,3,2-dioxaborinan-2-yl)phenyl)amino)-5-(trifluoromethyl)pyrimidin-4-yl)amino)tetrahydro-2H-pyran-4-carbonitrile (600 mg, crude) as a yellow solid.

### 49.4 Preparation of (trans)-3-((2-((7-chloro-1-hydroxy-1,3-dihydrobenzo[c][1,2]oxaborol-5-yl)amino)-5-(trifluoromethyl)pyrimidin-4-yl)amino)tetrahydro-2H-pyran-4-carbonitrile

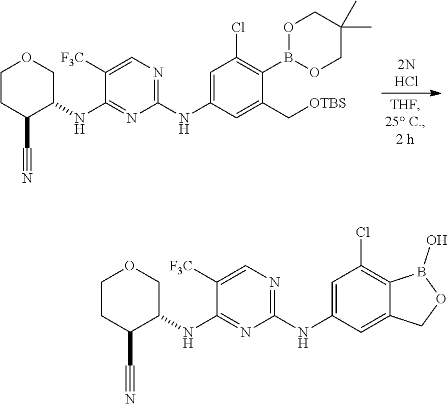

To a solution of (trans)-3-((2-((3-(((tert-butyldimethylsilyl)oxy)methyl)-5-chloro-4-(5,5-dimethyl-1,3,2-dioxaborinan-2-yl)phenyl)amino)-5-(trifluoromethyl)pyrimidin-4-yl)amino)tetrahydro-2H-pyran-4-carbonitrile (600 mg, 1.02 mmol, 1 eq) in THF (5 mL) was added aq. HCl (2 M, 10 mL) at 25° C. The mixture was stirred at 25° C. for 2 hr. Solid was precipitate out. TLC showed the reaction was complete. The reaction mixture was filtered and the filter cake was triturated with THF (5 mL) and filtered to give (trans)-3-((2-((7-chloro-1-hydroxy-1,3-dihydrobenzo[c][1,2]oxaborol-5-yl)amino)-5-(trifluoromethyl)pyrimidin-4-yl)amino)tetrahydro-2H-pyran-4-carbonitrile (7.3 mg) as a white solid. ¹H NMR (400 MHz, DMSO-d₆) δ=10.11 (s, 1H), 8.96 (s, 1H), 8.35 (s, 1H), 7.85-7.65 (m, 2H), 7.19 (d, J=9.2 Hz, 1H), 5.06-4.90 (m, 2H), 4.68-4.54 (m, 1H), 3.93-3.80 (m, 2H), 3.45-3.40 (m, 1H), 3.28-3.19 (m, 2H), 2.17-2.15 (m, 1H), 1.94-1.80 (m, 1H). MS (ESI): mass calcd. For C₁₈H₁₆BClF₃N₅O₃ 453.10, m/z found 452.0 [M−H]⁻. HPLC: 100% (220 nm), 100% (254 nm).

### 50. Preparation of (trans)-3-((5-chloro-2-((7 fluoro-1-hydroxy-1,3-dihydrobenzo[c][1,2]oxaborol-5-yl)amino)pyrimidin-4-yl)amino)tetrahydro-2H-pyran-4-carbonitrile

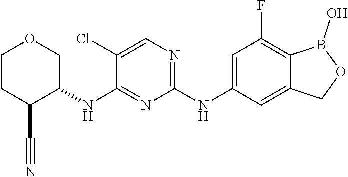

### 50.1 Preparation of (trans)-3-[[2-[4-bromo-3 fluoro-5-(hydroxymethyl)anilino]-5-chloro-pyrimidin-4-yl]amino]tetrahydropyran-4-carbonitrile

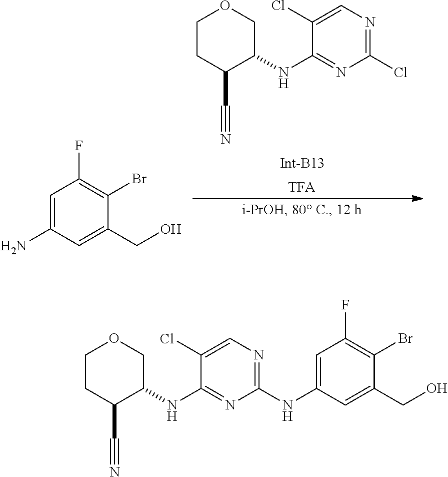 [SMILES: N#C[C@H]1CCOC[C@@H]1Nc1nc(Cl)ncc1Cl ; Nc1cc(F)c(Br)c(CO)c1 ; N#C[C@H]1CCOC[C@@H]1Nc1nc(Nc2cc(F)c(Br)c(CO)c2)ncc1Cl]

To a solution of (5-amino-2-bromo-3-fluoro-phenyl)methanol (450 mg, 2.05 mmol, 1 eq) in i-PrOH (10 mL) was added TFA (350 mg, 3.07 mmol, 1.5 eq) and (trans)-3-((2,5-dichloropyrimidin-4-yl)amino)tetrahydro-2H-pyran-4-carbonitrile (558 mg, 2.05 mmol, 1 eq) at 25° C. The mixture was stirred at 80° C. for 12 h. Solid was precipitate out. TLC showed the reaction was complete. The reaction mixture was filtered to give a residue. The residue was triturated with saturated aq. NaHCO₃ (10 mL) and filtered to give (trans)-3-[[2-[4-bromo-3-fluoro-5-(hydroxymethyl)anilino]-5-chloro-pyrimidin-4-yl]amino]tetrahydropyran-4-carbonitrile (600 mg, 64.2% yield) as a white solid.

### 50.2 Preparation of (trans)-3-((2-((4-bromo-3-(((tert-butyldimethylsilyl)oxy)methyl)-5-fluorophenyl)amino)-5-chloropyrimidin-4-yl)amino)tetrahydro-2H-pyran-4-carbonitrile

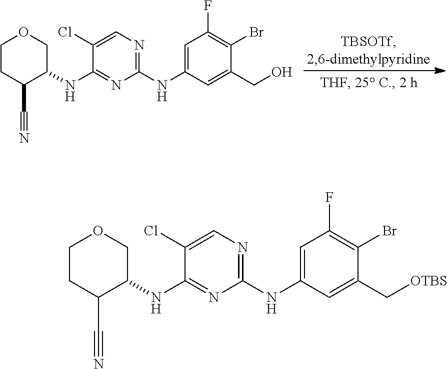 [SMILES: N#C[C@H]1CCOC[C@@H]1Nc1nc(Nc2cc(F)c(Br)c(CO)c2)ncc1Cl ; CC(C)(C)[Si](C)(C)OCc1cc(Nc2ncc(Cl)c(N[C@H]3COCCC3C#N)n2)cc(F)c1Br]

To a solution of 3-[[2-[4-bromo-3-fluoro-5-(hydroxymethyl)anilino]-5-chloro-pyrimidin-4-yl]amino]tetrahydropyran-4-carbonitrile (580 mg, 1.27 mmol, 1 eq) in THF (10 mL) was added 2,6-dimethylpyridine (544 mg, 5.08 mmol, 4 eq) and [tert-butyl(dimethyl)silyl]trifluoromethanesulfonate (503 mg, 1.90 mmol, 1.5 eq) at 25° C. The mixture was stirred at 25° C. for 2 h. TLC showed the reaction was complete. The reaction mixture was quenched by addition of H₂O (10 mL) at 0° C. and extracted with EtOAc (20 mL×3). The combined organic layers were washed with brine (20 mL×2), dried over Na₂SO₄, filtered, and concentrated under reduced pressure to give a residue. The residue was purified by column chromatography (SiO₂, Petroleum ether/ethyl acetate=10/1 to 5/1) to give 3-((2-((4-bromo-3-(((tert-butyldimethylsilyl) oxy)methyl)-5-fluorophenyl)amino)-5-chloropyrimidin-4-yl)amino)tetrahydro-2H-pyran-4-carbonitrile (650 mg, 89.6% yield) as a white solid. ¹H NMR (DMSO-d₆, 400 MHz) δ 9.82 (s, 1H), 8.29 (t, J=8.0 Hz, 1H), 8.06 (s, 1H), 7.89 (d, J=8.0 Hz, 1H), 7.61 (s, 1H), 4.67 (s, 2H), 4.52-4.42 (m, 1H), 3.98-3.80 (m, 2H), 3.38-3.28 (m, 2H), 3.25-3.15 (m, 1H), 2.35-2.05 (m, 1H), 1.95-1.82 (m, 1H), 0.93 (s, 9H), 0.13 (s, 6H).

### 50.3 Preparation of (trans)-3-((2-((3-(((tert-butyldimethylsilyl)oxy)methyl)-4-(5,5-dimethyl-1,3,2-dioxaborinan-2-yl)-5 fluorophenyl)amino)-5-chloropyrimidin-4-yl)amino)tetrahydro-2H-pyran-4-carbonitrile

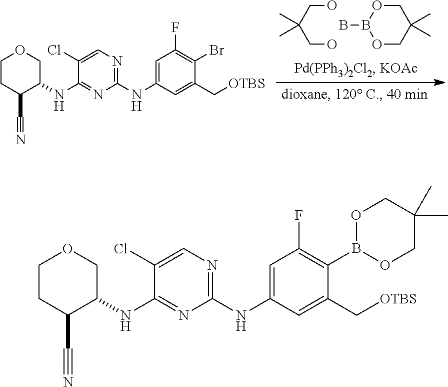

Two reactions were carried out in parallel. For each a solution of (trans)-3-((2-((4-bromo-3-(((tert-butyldimethylsilyl)oxy)methyl)-5-fluorophenyl)amino)-5-chloropyrimidin-4-yl)amino)tetrahydro-2H-pyran-4-carbonitrile (340 mg, 744 umol, 1 eq) in dioxane (8 mL) was added Pd(PPh₃)₂Cl₂ (52 mg, 74.4 umol, 0.1 eq), KOAc (182 mg, 1.86 mmol, 2.5 eq) and 2-(5,5-dimethyl-1,3,2-dioxaborinan-2-yl)-5,5-dimethyl-1,3,2-dioxaborinane (840 mg, 3.72 mmol, 5 eq) at 25° C. The mixture was stirred at 120° C. for 40 min. TLC showed the reaction was complete. The two parallel reactions were combined, filtered and the filtrate was concentrated under reduced pressure to give a residue. The residue was purified by column chromatography (SiO₂, Petroleum ether/ethyl acetate=3/1 to 1/1) to give (trans)-3-((2-((3-(((tert-butyldimethylsilyl) oxy)methyl)-4-(5,5-dimethyl-1,3,2-dioxaborinan-2-yl)-5-fluorophenyl)amino)-5-chloropyrimidin-4-yl)amino)tetrahydro-2H-pyran-4-carbonitrile (0.75 g, 88.2% yield) as a white solid.

### 50.4 Preparation of (trans)-3-((5-chloro-2-((7 fluoro-1-hydroxy-1,3-dihydrobenzo[c][1,2] oxaborol-5-yl)amino)pyrimidin-4-yl)amino)tetrahydro-2H-pyran-4-carbonitrile

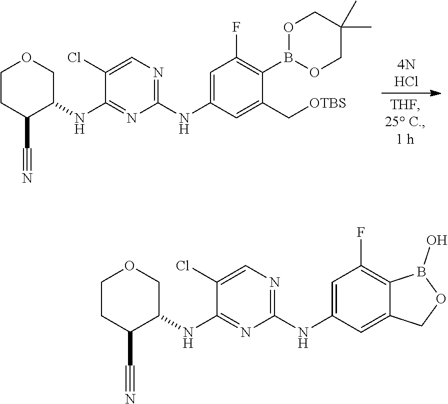

To a solution of (trans)-3-((2-((3-(((tert-butyldimethylsilyl)oxy)methyl)-4-(5,5-dimethyl-1,3,2-dioxaborinan-2-yl)-5-fluorophenyl)amino)-5-chloropyrimidin-4-yl)amino)tetrahydro-2H-pyran-4-carbonitrile (0.5 g, 828 umol, 1 eq) in THF (10 mL) was added aq. HCl (4 M, 2.0 mL, 10 eq) at 25° C. The mixture was stirred at 25° C. for 1 h. Solid was precipitate out. The reaction mixture was filtered to give a residue. The residue was triturated with THF (8 mL) and filtered to give 3-((5-chloro-2-((7-fluoro-1-hydroxy-1,3-dihydrobenzo[c][1,2]oxaborol-5-yl)amino)pyrimidin-4-yl)amino)tetrahydro-2H-pyran-4-carbonitrile (65.6 mg, 19.6% yield) as a white solid. ¹H NMR (DMSO-d₆, 400 MHz) δ 9.82 (s, 1H), 8.13 (s, 1H), 7.64 (s, 1H), 7.53-7.40 (m, 2H), 4.96 (s, 2H), 4.52-4.42 (m, 1H), 3.98-3.80 (m, 2H), 3.38-3.28 (m, 2H), 3.25-3.15 (m, 1H), 2.35-2.05 (m, 1H), 1.95-1.82 (m, 1H). MS (ESI): mass calcd. For C₁₇H₁₆BClFN₅O₃ 403.10, m/z found 404.1 [M+H]⁺. HPLC: 99.04% (220 nm), 98.94% (254 nm).

### Comparator Compounds

Comparator compounds which are derivatives of Compound (II) having Y¹⁻⁴ substituents (compounds C001-C020 as listed below) were prepared and the biological testing results for the comparator compounds are provided in Table 3b (Biological Examples). The comparator compounds may be made using the starting materials and synthetic procedures as outlined in PCT Application No. PCT/US2020/070234, which published as WO 2021/003501, and which is herein incorporated by reference with regard to such teaching.

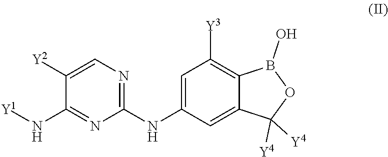

| Comparator ID | Y¹ | Y² | Y³ | Y⁴ |
|---|---|---|---|---|
| C001 | (Et)₂CH— | Me | H | H |
| C002 | (Et)₂CH— | Me | Cl | H |
| C003 | (Et)₂CH— | Me | F | H |
| C004 | (Et)₂CH— | Me | Me | H |
| C005 | (Et)₂CH— | Cl | H | H |
| C006 | Cyclopentyl- | Me | H | H |
| C007 | Cyclopentyl- | Me | Cl | H |
| C008 | Cyclopentyl- | Me | Me | H |
| C009 | Cyclopentyl- | Me | H | Me |
| C010 | Cyclohexyl- | H | H | H |
| C011 | Cyclohexyl- | Me | H | H |
| C012 | Cyclohexyl- | Me | Cl | H |
| C013 | Cyclopentyl- | F | H | H |
| C014 | Cyclopentyl- | Cl | H | H |
| C015 | Cyclopentyl- | Cl | F | H |
| C016 | Cyclopentyl- | Cl | Me | H |
| C017 | Cyclohexyl- | F | H | H |
| C018 | Cyclohexyl- | Cl | H | H |
| C019 | Cyclopentyl- | CF₃ | H | H |
| C020 | Cyclohexyl- | CF₃ | H | H |

### Biological Examples

The compounds of the present disclosure were tested in multiple assays as described below. Representative results from the compounds of the present disclosure are compiled in Table 3a. Results for Comparative Examples are provided in Table 3b.

### Biochemical Kinase Assay Protocol (JAK)

Reagent: Base Reaction buffer; 20 mM Hepes (pH 7.5), 10 mM MgCl₂, 1 mM EGTA, 0.02% Brij™ 35, 0.02 mg/ml BSA, 0.1 mM Na₃VO₄, 2 mM DTT, 1% DMSO, where required cofactors were added individually to each kinase reaction.

Reaction Procedure:
1. Prepared indicated substrate in freshly prepared Base Reaction Buffer
2. Delivered any required cofactors to the substrate solution above
3. Delivered indicated kinase into the substrate solution and gently mix
4. Delivered compounds in DMSO into the kinase reaction mixture by Acoustic technology (Echo550; nanoliter range), incubated for 20 minutes at room temperature
5. Delivered 33P-ATP into the reaction mixture to initiate the reaction.
6. Incubated kinase reaction for 2 hours at room temperature
7. Reactions were spotted onto P81 ion exchange paper
8. Detected kinase activity by filter-binding method.

### Cytokine Inhibition Assay Protocol for IL-4 and IL-31

The test compounds are solubilized in DMSO, then diluted to make appropriate stocks for use in the assay, and diluted in culture medium to 20× assay concentrations. PBMC's are plated and allowed to settle for 1 hour at 37° C., 5% CO₂. Test compounds and controls are added to the settled PBMC's and incubated for 1 hour at 37° C., 5% CO₂. The PBMC's are then be treated with PHA (10 μg/mL) and incubated for 24 hours at 37° C., 5% CO₂. DMSO is used as a positive control and dexamethasone (100 nM) was used as a reference inhibitor control. After the main incubation, cell culture supernatants are harvested and assayed for the cytokines listed above, using standard Luminex protocols. Levels of cytokine induction are interpolated from standard curves using 5-parameter non-linear regression analyses, where y=(A+((B−A)/(1+(((B−E)/(E−A))*((x/C)\hat{ }D))))). The interpolated data is normalized to DMSO controls and analyzed to determine IC₅₀ values using 4-parameter non-linear regression analyses, where y=(A+((B−A)/(1+((C/x)\hat{ }D))))

### Cytokine Function Assay Protocols for IL-4/pSTAT6 and GM-CSF/pSTAT5

GM-CSF/pSTAT5:
Whole blood from a healthy donor was lysed to remove red blood cells. Cells were plated onto a 96 well plate. Compound was added and incubated for 1 hour (at 37° C.). After 1 hour, cells were stimulated with GM-CSF for 15 minutes. Cells were fixed and stained with anti-pSTAT5 antibody. After staining, cells were read on a Beckman-Coulter CytoFLEX.

IL-4/pSTAT6:
PBMC from a healthy donor was plated onto a 96 w plate. Compound was added and incubated for 1 hour (at 37° C.). After 1 hour, cells were stimulated with IL-4 for 15 minutes. Cells were fixed and stained with anti-pSTAT6 antibody. After staining, cells were read on a Beckman-Coulter CytoFLEX.

### Cytokine Function Assay Protocol for IL-31

The protocol for the cytokine function assay for IL-31 is set forth below:
1. Plate DH82 cells at a density of 125,000 cells per well in a 96-well plate and incubate overnight in EMEM medium (ATCC® 30-2003) containing 15% FBS and 10 ng/mL canine IFNr (R&D, Cat #781-CG-050, Lot #DIA1320011) at 37° C.
2. After 24 hr, wash with 37° C. pre-warmed PBS (100 uL/well).
3. Add serum-free MEM, 90 uL per well, 2 hr, 37° C.
4. Add 10 uL 10×IL-31 (40, 10, 2.5 ug/mL) in duplicate, 5 min, 37° C.
5. Remove medium, add 60 uL lysis buffer per well. Agitate on a plate shaker (˜350 rpm) for 10 min at RT.
6. Add 12 uL lysate/50% lysate to 384-well Opaque assay plate, 25% control lysate and lysis buffer were added to the assay plate in duplicate as positive and negative controls.
7. Add 6 uL Acceptor Mix, cover with aluminum foil, quickly spin at 1000 rpm, shake at 120 rpm for 2 min, then incubate at RT for 1 hr.
8. Add 6 uL Donor Mix, cover with aluminum foil, quickly spin at 1000 rpm, shake at 120 rpm for 2 min, then incubate at RT for 1 hr.
9. Measure the fluorescence using EnVision™ plate reader.

### Dog LM (Liver Microsome) Assay

The metabolic degradation of the test compound is assayed at 37° C. with pooled liver microsomes from dogs (Beagle). The final incubation volume of 100 μl per time point contains TRIS buffer pH 7.6 at RT (0.1 M), magnesium chloride (5 mM), microsomal protein (1 mg/ml) and the test compound at a final concentration of 1 μM. Following a short preincubation period at 37° C., the reactions were initiated by addition of beta-nicotinamide adenine dinucleotide phosphate, reduced form (NADPH, 1 mM) and terminated by transferring an aliquot into solvent after different time points. Additionally, the NADPH-independent degradation was monitored in incubations without NADPH, terminated at the last time point. The [%] remaining test compound after NADPH independent incubation is reflected by the parameter c (control) (metabolic stability). The quenched incubations are pelleted by centrifugation (10000 g, 5 min). An aliquot of the supernatant is assayed by LC-MS/MS for the amount of parent compound. The half-life (t½ INVITRO) is determined by the slope of the semilogarithmic plot of the concentration-time profile. The intrinsic clearance (CL_INTRINSIC) is calculated by considering the amount of protein in the incubation: CL_INTRINSIC [μl/min/mg protein]=(Ln 2/(half-life [min]*protein content [mg/ml]))*1000. For better across species comparison the predicted clearance is expressed as percent of the liver blood flow [% QH] in the individual species. In general, high stability (corresponding to low % QH) of the compounds across species is desired.

### Test Compounds

The results of biological testing for the compounds according to the present disclosure are provided below in Table 3a. The Example Number entries formatted ##a represent stereoisomer one and entries formatted ##b represent stereoisomer two. A recitation of “-” is intended as a designation of a compound of the present disclosure that is not yet tested for the respective assay.

TABLE 3a
Biological Testing Results

| | IC₅₀ | IC₅₀ | IC₅₀ | IC₅₀ | IC₅₀ | IC₅₀ | Dog LM |
|---|---|---|---|---|---|---|---|
| Example | JAK1 | JAK2 | PDE4B | IL-4 | GM-CSF | IL-31 | QH |
| Number | (nM) | (nM) | (nM) | (nM) | (nM) | (nM) | (%) |
| 001 | 1.1 | 24.7 | 362.0 | 173.0 | — | — | 39.7 |
| 002 | 0.9 | 12.1 | 832.0 | 94.8 | 2101 | 23.9 | <19.9 |
| 003 | 0.7 | 40.0 | — | 106.4 | 1430 | 21.2 | <19.9 |
| 003a | 0.7 | 3.7 | — | 93.3 | 2225 | 9.2 | <19.9 |
| 003b | 5.0 | 83.1 | — | 74.8 | — | — | <19.9 |
| 004 | 1.6 | 49.7 | — | — | — | — | — |
| 005 | 0.5 | 9.0 | 44.5 | — | — | — | <19.9 |
| 006 | 1.0 | 36.6 | — | 201.9 | — | — | <19.9 |
| 007 | 5.3 | 52.6 | — | — | — | — | — |
| 008 | 0.3 | 3.4 | — | 95.9 | 774 | 40.2 | <19.9 |
| 009 | 1.1 | 13.8 | — | — | — | — | <19.9 |
| 010 | 0.7 | 19.3 | — | 71.7 | 1018 | 18.1 | <19.9 |
| 010a | 0.6 | 4.0 | — | 37.9 | 469 | 12.7 | <19.9 |
| 010b | 22.3 | 811.1 | — | — | — | — | <19.9 |
| 011 | 0.9 | 37.5 | — | 159.1 | 1150 | 33.6 | <19.9 |
| 011a | 0.4 | 11.7 | — | 151.4 | 1485 | 106.9 | <19.9 |
| 011b | 10.0 | 196.7 | — | — | — | — | <19.9 |
| 012a | 0.6 | 2.4 | — | — | — | — | <19.9 |
| 012b | 8.2 | 126.1 | — | — | — | — | <19.9 |
| 013 | 2.6 | 100.8 | — | — | — | — | — |
| 014 | 1.4 | 6.3 | — | — | — | — | 48.7 |
| 015 | 9.0 | 103.4 | — | — | — | — | — |
| 016 | 2.5 | 17.5 | — | — | — | — | 70.2 |
| 017 | 0.4 | 4.2 | — | 233.0 | 10000 | 35.4 | <19.9 |
| 018 | 0.5 | 10.4 | — | — | — | — | <19.9 |
| 019 | 0.2 | 3.2 | | | | | |
| 020 | 1.4 | 50.7 | | | | | |
| 021 | 2.7 | 3.2 | | | | | |
| 022 | 3.3 | 7.5 | | | | | |
| 023 | 0.2 | 0.6 | | | | | |
| 024 | 5.9 | 11.2 | | | | | |
| 025 | 0.4 | 24.2 | | | | | <19.9 |
| 026 | 0.5 | 7.0 | — | 91.9 | 2662 | 64.6 | <19.9 |
| 026a | 0.4 | 1.6 | 972.0 | 49.7 | 4240 | 21.6 | <19.9 |
| 026b | 47.1 | 95.3 | — | — | — | — | <19.9 |
| 027 | 0.6 | 3.5 | — | 79.9 | 1204 | 81.3 | 30.1 |
| 028 | 0.5 | 3.5 | — | — | — | — | — |
| 029 | 0.2 | 9.8 | 978.0 | 58.3 | 2240 | 49.5 | <19.9 |
| 029a | 0.02 | 3.7 | 381.0 | 48.84 | 1166 | 19.8 | <19.9 |
| 029b | 4.0 | 89.8 | — | — | — | — | <19.9 |
| 030a | 51.1 | 868.5 | — | — | — | — | <19.9 |
| 030b | 4.7 | 91.9 | — | 192.8 | 4483 | 25.8 | <19.9 |
| 031 | 1.2 | 40.8 | — | 67.5 | 3150 | 27.6 | <19.9 |
| 031a | 55.4 | 665.8 | — | | — | — | <19.9 |
| 031b | 0.5 | 9.9 | — | 67.9 | 1612 | 20.2 | <19.9 |
| 032 | 0.6 | 3.3 | 762.0 | 66.7 | 901 | 51.0 | 42.0 |
| 033a | 0.4 | 8.1 | — | 136.5 | 1704 | 19.1 | <19.9 |
| 033b | 16.3 | 389.4 | — | — | — | — | 64.9 |
| 034 | 1.1 | 33.7 | — | — | — | — | 74.7 |
| 035 | 0.7 | 8.8 | 320.0 | — | — | — | 43.7 |
| 036a | 0.3 | 4.8 | — | 158.2 | — | — | 33.1 |
| 036b | 8.2 | 102.2 | — | — | — | — | 37.3 |
| 037 | 3.1 | 18.5 | — | 919.4 | — | — | 19.9 |
| 038 | 1.4 | 23.7 | — | — | — | — | 50.4 |
| 039a | 1.1 | 18.9 | — | 264.8 | — | — | 78.4 |
| 039b | 216.3 | 2043.0 | — | — | — | — | 72.1 |
| 040a | >1000 | >1000 | — | — | — | — | 32.9 |
| 040b | 1.1 | 16.7 | — | 1407.0 | — | — | 47.4 |
| 041a | 0.8 | 3.1 | — | 47.0 | >10,000 | 40.0 | <19.9 |
| 041b | 36.2 | 133.7 | — | — | — | | <19.9 |
| 042a | 0.4 | 7.3 | — | 148.4 | 2972 | 9.7 | <19.9 |
| 042b | 98.2 | 690.0 | — | — | — | | <19.9 |
| 043a | 0.9 | 23.7 | — | 267.1 | 4140 | 45.4 | <19.9 |
| 043b | 7.6 | 298.2 | — | — | — | — | <19.9 |
| 044a | 0.2 | 0.8 | | | | | 34.9 |
| 044b | 33.8 | 96.8 | | | | | <19.9 |
| 045 | 0.5 | 10.6 | — | — | — | — | — |
| 046 | 3.4 | 186 | | | | | |
| 047 | 0.34 | 7.6 | | | | | |
| 048 | 0.6 | 15.6 | | | | | |
| 049 | 8.2 | 13.3 | 362.0 | 173.0 | — | — | 39.7 |
| 050 | 0.2 | 1.2 | 832.0 | 94.8 | 2101 | 23.9 | <19.9 |

Biological testing results for comparator compounds of structure (II) with variables Y¹, Y², Y³, and Y⁴ are provided in Table 3b:

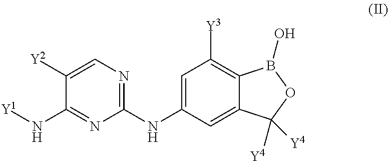

TABLE 3b
Biological Testing Results, comparator compounds

| | |
|---|---|
| | (II) |
| 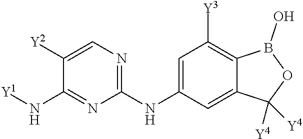 | |

| | | | | | | | Inhibition | | | | | |
|---|---|---|---|---|---|---|---|---|---|---|---|---|
| | | | | | IC₅₀ | IC₅₀ | PDE4B | IC₅₀ | IC₅₀ | IC₅₀ | IC₅₀ | |
| Comparator | | | | | JAK1 | JAK2 | @ 1 μM | PDE4B | IL-4 | GM-CSF | IL-31 | Dog LM |
| ID | Y¹ | Y² | Y³ | Y⁴ | (nM) | (nM) | (%) | (nM) | (nM) | (nM) | (nM) | QH(%) |
| C001 | (Et)₂CH— | Me | H | H | 1.6 | 60.2 | 90 | 92.2 | 115.0 | 1718 | — | 73.8 |
| C002 | (Et)₂CH— | Me | Cl | H | 1.1 | 95.7 | — | — | 155.0 | 10000 | — | <19.9 |
| C003 | (Et)₂CH— | Me | F | H | 0.7 | 43.7 | — | 212.0 | 125.0 | 1156 | — | 41.0 |
| C004 | (Et)₂CH— | Me | Me | H | 1.1 | 41.0 | 22 | — | 118.0 | >10000 | — | 34.5 |
| C005 | (Et)₂CH— | Cl | H | H | 1.1 | 33.3 | — | 379.0 | 6119.0 | 2254 | — | — |
| C006 | Cyclopentyl- | Me | H | H | 6.5 | 154.0 | 86 | 114.0 | 293.0 | 270 | — | >86.6 |
| C007 | Cyclopentyl- | Me | Cl | H | 0.7 | 31.8 | — | 113.0 | 300.1 | 3742 | — | 52.1 |
| C008 | Cyclopentyl- | Me | Me | H | 2.3 | 59.3 | 37 | — | 973.0 | >10000 | — | 54.3 |
| C009 | Cyclopentyl- | Me | H | Me | 2.4 | 83.9 | 40 | 761.0 | 218.0 | 4221 | — | 83.4 |
| C010 | Cyclohexyl- | H | H | H | 48.0 | 364.0 | 53 | 956.0 | — | — | — | — |
| C011 | Cyclohexyl- | Me | H | H | 3.2 | 32.9 | 13 | 242.0 | 2494.0 | 7704 | — | 83.5 |
| C012 | Cyclohexyl- | Me | Cl | H | 0.1 | 6.5 | — | — | 223.5 | 1783 | — | <19.9 |
| C013 | Cyclopentyl- | F | H | H | 32.3 | 705.0 | — | — | — | — | — | — |
| C014 | Cyclopentyl- | Cl | H | H | 3.3 | 179.0 | — | 52.7 | 782.0 | 5354 | — | 78.5 |
| C015 | Cyclopentyl- | Cl | F | H | 0.8 | 26.5 | — | 85.7 | — | — | — | 67.2 |
| C016 | Cyclopentyl- | Cl | Me | H | 2.1 | 663.0 | 30 | — | 688.0 | 927 | — | 54.5 |
| C017 | Cyclohexyl- | F | H | H | 11.2 | 1850.0 | — | — | 1638.0 | 10000 | — | 53.5 |
| C018 | Cyclohexyl- | Cl | H | H | 6.1 | 118.0 | — | 108.0 | — | — | — | 56.0 |
| C019 | Cyclopentyl- | CF₃ | H | H | 29.5 | 171.0 | 90 | 275.0 | — | — | — | 73.8 |
| C020 | Cyclohexyl- | CF₃ | H | H | 44.1 | 397.0 | — | 37.6 | — | — | — | <19.9 |

Summary of Pharmacokinetic (PK) profile

Three healthy male Beagle dogs were studied to determine a representative pharmacokinetic (PK) profile following a single oral administration of 1.0 mg/kg to each dog in a fasted state. Blood samples were collected at pre-dose, 0.25, 0.5, 1, 2, 4, 8, 24; 32, and 48 h post dose following oral administration (1 mg/mL in 10% Propylene glycol (PG), 45% PEG400, 7% Cremophor EL, 38% 50 mM citrate buffer pH 3.0 suspension or clear solution).

TABLE 4

| | | Median | Mean | Median | Mean | Mean | Mean | Mean |
|---|---|---|---|---|---|---|---|---|
| | Dose | Tₘₐₓ | Cₘₐₓ | Tₗₐₛₜ | Cₗₐₛₜ | AUCₗₐₛₜ | AUC_{INF}_{_{—}}_{obs} | T_{1/2} |
| Analyte | (mg/kg) | (h) | (ng/mL) | (h) | (ng/mL) | (h*ng/mL) | (h*ng/mL | (h) |
| Example | 1.0 | 1.0 | 279 | 48 | 4.74 | 2460 | 2560 | 14.6 |
| 33a | | | | | | | | |
| Example | 1.01 | 2.67 | 445 | 48 | 7.98 | 3667 | 3807 | 11.4 |
| 31b | | | | | | | | |

### Summary of Effectiveness

Ten healthy adult male Beagle dogs with no previous history of pruritus or skin disease were selected. All dogs first received intravenously recombinant canine IL-31 (1.75 ug/kg) to evaluate increased pruritic behaviors without any medications (IL-31 alone). Afterward, all dogs received repeated administration of oral solution and again intravenously recombinant canine IL-31 injections to assess inhibition of IL-31 induced itch behavior. (See, e.g., Gonzales, Vet. Dermatol. 2016, 27, 34-e10 and Pearson, Vet. Sci. 2023, 10, 369)

An oral solution of a representative compound of the present disclosure, Example 33a, at 1 mg/kg once daily in the morning for five (5) days, was administered to each dog in a crossover design for the five (5) consecutive days. All dogs were fasted during the oral administration and thirty minutes (30 min) post oral drug administration. On day 5, all dogs were administered a recombinant canine IL-31 intravenously at the dose of 1.75 μg/kg.

A comparative analysis between treatments (IL-31 baseline/alone, and representative compound/Example 33a) revealed that the representative compound of the present disclosure showed a significant anti-pruritic effect when compared to IL-31 baseline/alone.

In some embodiments, the compounds of the present disclosure are potent JAK inhibitors. As such, the compounds may be used for the treatment or control of inflammation, auto-immune diseases, cancer, and other disorders and indications where modulation of JAK would be desirable.

All publications, patents, and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present disclosure.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

The present disclosure is further supplemented by the following numbered paragraphs:

#1. A compound of formula I:

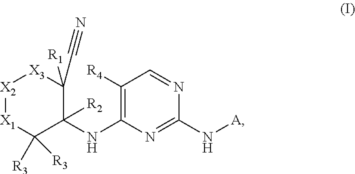

or a pharmaceutically acceptable salt or a stereoisomer or tautomer thereof, wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R₁ is selected from the group consisting of hydrogen, halogen (when X₃ is —C(R⁶)(R⁷)), hydroxy, C₁₋₆ alkyl, C₃₋₇ cycloalkyl, C₁₋₆ alkyl(oxy), and C₃₋₇ cycloalkyl(oxy), wherein the last four members of the of the above-mentioned R¹ group may optionally be substituted independently of one another with one or more halogen;
R₂ is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R₃ is independently selected from the group consisting of hydrogen, halogen (when X₁ is —C(R⁶)(R⁷)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of: hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl;
A is selected from the group consisting of benzo[c][1,2]oxaborol-1(3H)-ol, 3,4-dihydro-1Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇:

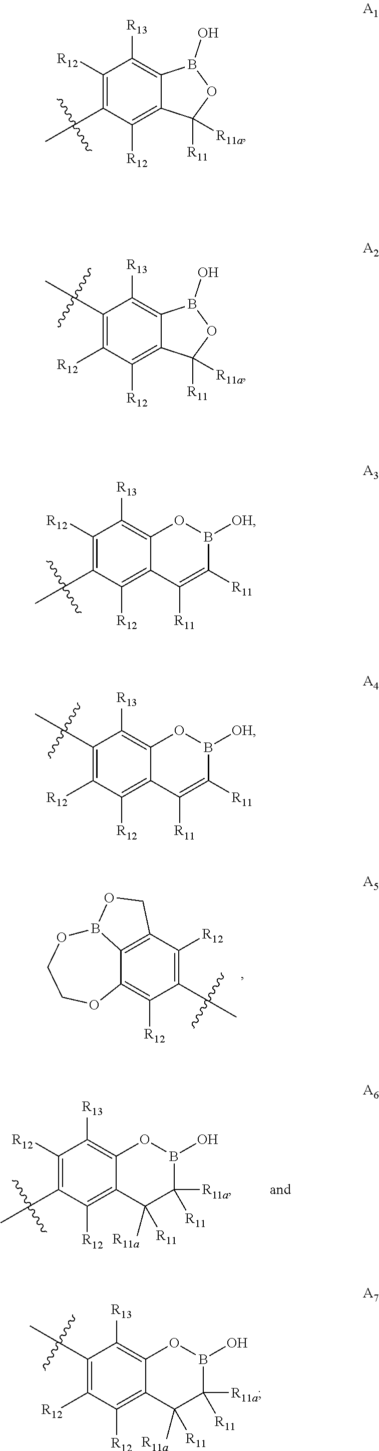

R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇ cycloalkyl, C₁₋₆ alkyl(oxy), C₃₋₇ cycloalkyl(oxy), COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁-3-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl); wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

#2. A compound according to paragraph 1 wherein
X₁ is —C(R⁶)(R⁷)—;
X₂ is selected from —C(R⁶)(R⁷)—, or X₂ comprises a bond between X₁ and X₃;
X₃ is-C(R⁶)(R⁷)—
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of: hydrogen, halogen, C₁-C₃ alkyl, and C₁-C₃ haloalkyl;
R⁶ and R⁷ are independently selected from the group consisting of hydrogen, —C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring;
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

#3. A compound according to one or more of paragraphs 1 to 2, wherein
X₁ is selected from the group consisting of —O—, and —C(R⁶)(R⁷)—;
X₂ is selected from the group consisting of —O—, and —C(R⁶)(R⁷)—, or
   X₂ comprises a bond between X₁ and X₃;
X₃ is —C(R⁶)(R⁷)—, except X₁ and X₂ cannot both be —O—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of hydrogen, halogen, C₁-C₃ alkyl, and C₁-C₃ haloalkyl;
R⁶ and R⁷ are independently selected from the group consisting of hydrogen, —C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring;
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

#4. The compound according to any one or more of paragraphs 1-3, wherein
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

#5. The compound according to any one or more of paragraphs 1-4, wherein
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

#6. A compound of formula (Ia) or (Ib):

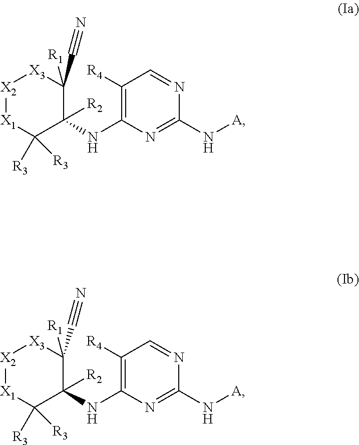

or a pharmaceutically acceptable salt or a stereoisomer or a tautomer thereof
wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or
X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R¹ is selected from the group consisting of hydrogen, halogen (when X₃ is —C(R⁷)(R⁸)—), hydroxy, C₁₋₆ alkyl, C₃₋₇ cycloalkyl, C₁₋₆ alkyl(oxy), and C₃₋₇ cycloalkyl(oxy), wherein the last four members of the of the above-mentioned R¹ group may optionally be substituted independently of one another with one or more halogen;
R² is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R³ is independently selected from the group consisting of hydrogen, halogen (when X₁ is —C(R⁷)(R⁸)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl;
A is selected from the group consisting of benzo[c][1,2]oxaborol-1(3H)-ol, 3,4-dihydro-1Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇:

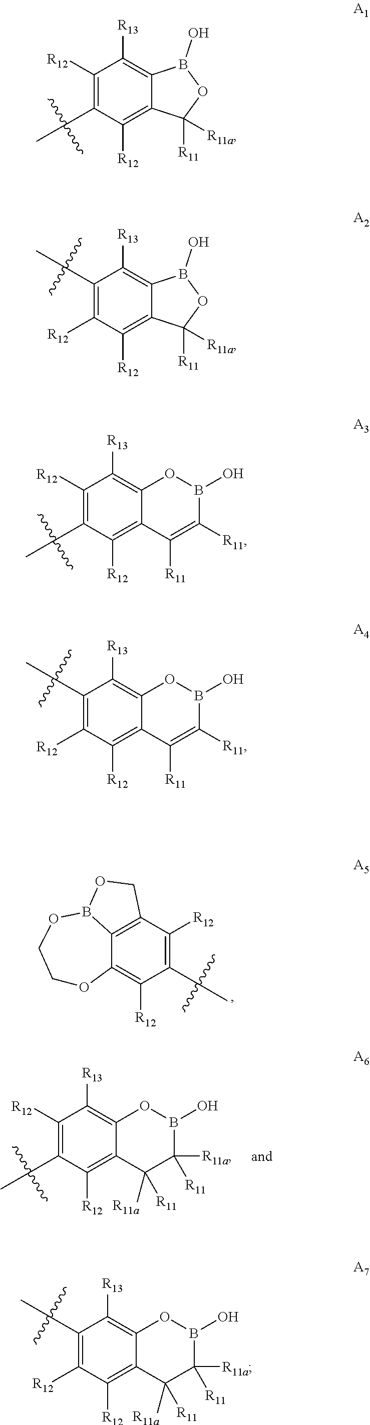

R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇ cycloalkyl, C₁₋₆ alkyl(oxy), C₃₋₇ cycloalkyl(oxy), COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

#7. A compound according to paragraph 6, wherein
X₁ is —C(R⁶)(R⁷)—;
X₂ is selected from —C(R⁶)(R⁷)—, or X₂ comprises a bond between X₁ and X₃;
X₃ is —C(R⁶)(R⁷)—
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of hydrogen, halogen, C₁-C₃ alkyl, and C₁-C₃ haloalkyl;
R⁶ and R⁷ are independently selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring; and
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

#8. The compound according to one or more of paragraphs 6-7, wherein
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R₄ is selected from the group consisting of hydrogen, halogen, C₁-C₃ alkyl, and C₁-C₃ haloalkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

#9. The compound according to any one or more of paragraphs 6-8, wherein
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

#10. A compound selected from the group shown in the following Table 1:

TABLE 1

| Example | |
|---|---|
| Number | Structure |
| 1 | 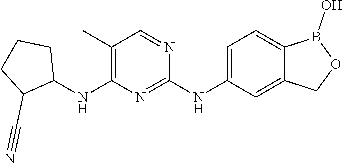 [SMILES: Cc1cnc(Nc2ccc3c(c2)COB3O)nc1NC1CCCC1C#N] |
| 2 | 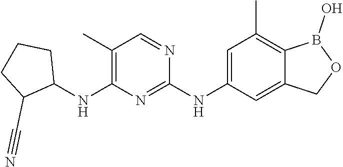 [SMILES: Cc1cnc(Nc2cc(C)c3c(c2)COB3O)nc1NC1CCCC1C#N] |
| 3 | 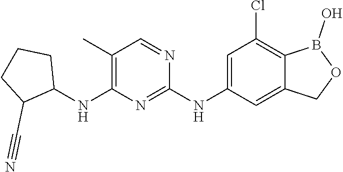 [SMILES: Cc1cnc(Nc2cc(Cl)c3c(c2)COB3O)nc1NC1CCCC1C#N] |
| 4 | 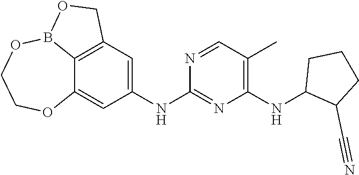 [SMILES: Cc1cnc(Nc2cc3c4c(c2)OCCOB4OC3)nc1NC1CCCC1C#N] |
| 5 | 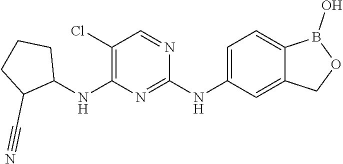 [SMILES: N#CC1CCCC1Nc1nc(Nc2ccc3c(c2)COB3O)ncc1Cl] |
| 6 | 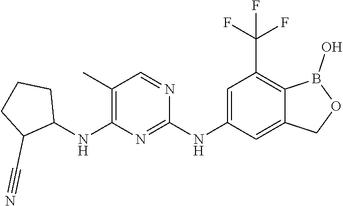 [SMILES: Cc1cnc(Nc2cc3c(c(C(F)(F)F)c2)B(O)OC3)nc1NC1CCCC1C#N] |
| 7 | 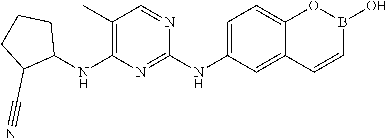 [SMILES: Cc1cnc(Nc2ccc3c(c2)C=CB(O)O3)nc1NC1CCCC1C#N] |
| 8 | 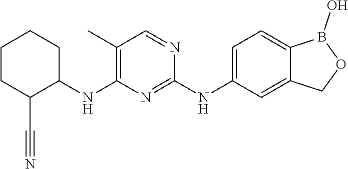 [SMILES: Cc1cnc(Nc2ccc3c(c2)COB3O)nc1NC1CCCCC1C#N] |
| 9 | 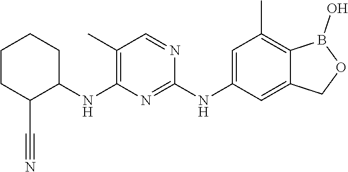 [SMILES: Cc1cnc(Nc2cc(C)c3c(c2)COB3O)nc1NC1CCCCC1C#N] |
| 10 | 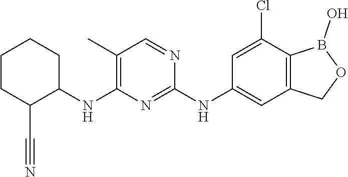 [SMILES: Cc1cnc(Nc2cc(Cl)c3c(c2)COB3O)nc1NC1CCCCC1C#N] |
| 11 | 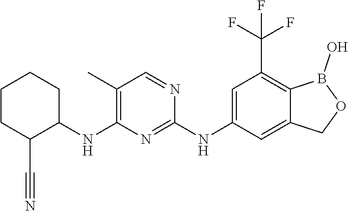 [SMILES: Cc1cnc(Nc2cc3c(c(C(F)(F)F)c2)B(O)OC3)nc1NC1CCCCC1C#N] |
| 12 | 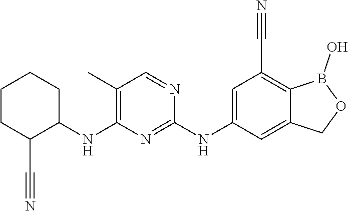 [SMILES: Cc1cnc(Nc2cc(C#N)c3c(c2)COB3O)nc1NC1CCCCC1C#N] |
| 13 | 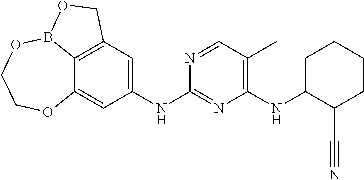 [SMILES: Cc1cnc(Nc2cc3c4c(c2)OCCOB4OC3)nc1NC1CCCCC1C#N] |
| 14 | 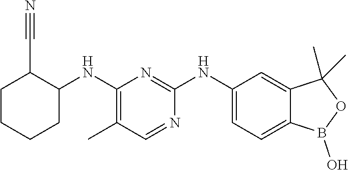 [SMILES: Cc1cnc(Nc2ccc3c(c2)C(C)(C)OB3O)nc1NC1CCCCC1C#N] |
| 15 | 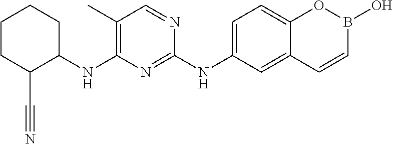 [SMILES: Cc1cnc(Nc2ccc3c(c2)C=CB(O)O3)nc1NC1CCCCC1C#N] |
| 16 | 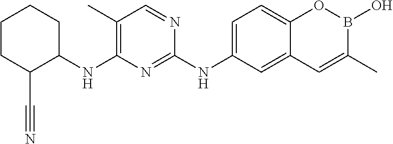 [SMILES: CC1=Cc2cc(Nc3ncc(C)c(NC4CCCCC4C#N)n3)ccc2OB1O] |
| 17 | 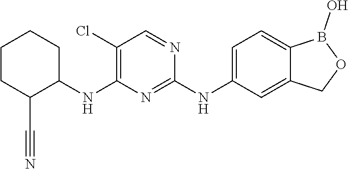 [SMILES: N#CC1CCCCC1Nc1nc(Nc2ccc3c(c2)COB3O)ncc1Cl] |
| 18 | 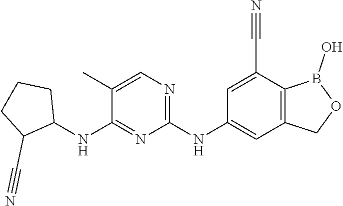 [SMILES: Cc1cnc(Nc2cc(C#N)c3c(c2)COB3O)nc1NC1CCCC1C#N] |
| 19 | 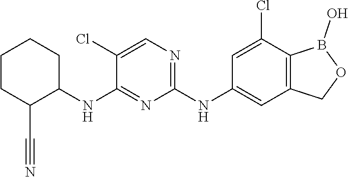 [SMILES: N#CC1CCCCC1Nc1nc(Nc2cc(Cl)c3c(c2)COB3O)ncc1Cl] |
| 20 | 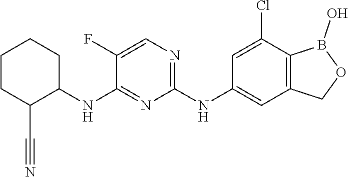 [SMILES: N#CC1CCCCC1Nc1nc(Nc2cc(Cl)c3c(c2)COB3O)ncc1F] |
| 21 | 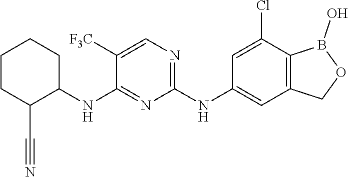 [SMILES: N#CC1CCCCC1Nc1nc(Nc2cc(Cl)c3c(c2)COB3O)ncc1C(F)(F)F] |
| 22 | 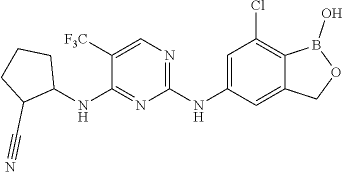 [SMILES: N#CC1CCCC1Nc1nc(Nc2cc(Cl)c3c(c2)COB3O)ncc1C(F)(F)F] |
| 23 | 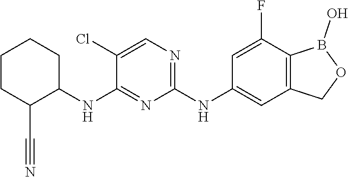 [SMILES: N#CC1CCCCC1Nc1nc(Nc2cc(F)c3c(c2)COB3O)ncc1Cl] |
| 24 | 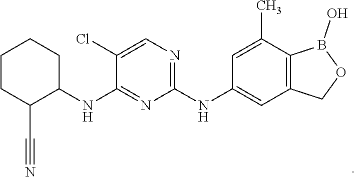 [SMILES: Cc1cc(Nc2ncc(Cl)c(NC3CCCCC3C#N)n2)cc2c1B(O)OC2] |

#11. The compound according to paragraph 6, wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or
   X₂ comprises a bond between X₁ and X₃;
X₃ is —C(R⁶)(R⁷)—, except X₁ and X₂ cannot both be —O—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned group may optionally be substituted independently of one another with one or more halogen;
R₄ is selected from the group consisting of halogen, C₁-C₃ alkyl, and C₁-C₃ haloalkyl;
R⁶ and R⁷ are independently selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned group may optionally be substituted independently of one another by one or more substituents selected from the group consisting of halogen and hydroxy, and R⁶ and R⁷ may be linked together to form a ring; and
each R¹² is independently selected from the group consisting of hydrogen, halogen, and C₁₋₆ alkyl.

#12. The compound according to one or more of paragraphs 6 and 11, wherein
X₁ is selected from the group consisting of —O—, —N(R⁵)—, and —C(R⁶)(R⁷)—;
X₂ is selected from the group consisting of —O— and —C(R⁶)(R⁷)—, except X₁ and X₂ may not both be —O—, or X₂ comprises a bond between X₁ and X₃;
X₃ is —C(R⁶)(R⁷)—;
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl is optionally substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members said R¹³ group may optionally be substituted independently of one another by one or more substituents selected from the group consisting of halogen and hydroxy.

#13. The compound according to one or more of paragraphs 11 to 12, wherein
X₂ is —C(R⁶)(R⁷)—;
X₃ is —C(R⁶)(R⁷)—;
R¹, R², and R³, are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl is optionally substituted with one or more of halogen;
each R¹¹ is independently selected from the group consisting of hydrogen, halogen, C₁₋₆ alkyl, and C₃₋₇ cycloalkyl, wherein the last two members of the above-mentioned R¹¹ group may optionally be substituted independently of one another with one or more halogen;
each R^{11a} is independently selected from the group consisting of hydrogen, halogen, C₁₋₆ alkyl, and C₃₋₇ cycloalkyl, wherein the last two members of the above-mentioned R¹¹ group may optionally be substituted independently of one another with one or more halogen;
each R¹² is hydrogen; and
each R¹³ is independently selected from the group consisting hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

#14. A compound selected from the group shown in the following Table 2:

TABLE 2

| Example | |
|---|---|
| Number | Structure |
| 25 | 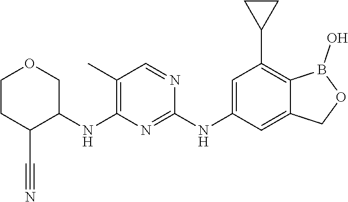 [SMILES: Cc1cnc(Nc2cc3c(c(C4CC4)c2)B(O)OC3)nc1NC1COCCC1C#N] |
| 26 | 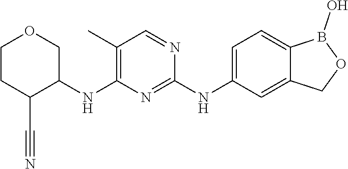 [SMILES: Cc1cnc(Nc2ccc3c(c2)COB3O)nc1NC1COCCC1C#N] |
| 27 | 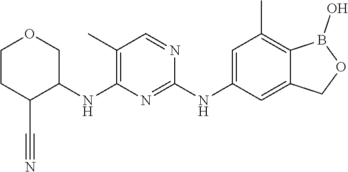 [SMILES: Cc1cnc(Nc2cc(C)c3c(c2)COB3O)nc1NC1COCCC1C#N] |
| 28 | 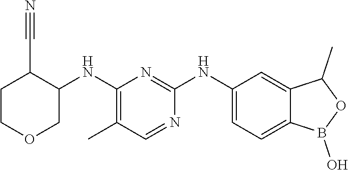 [SMILES: Cc1cnc(Nc2ccc3c(c2)C(C)OB3O)nc1NC1COCCC1C#N] |
| 29 | 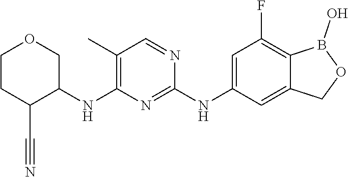 [SMILES: Cc1cnc(Nc2cc(F)c3c(c2)COB3O)nc1NC1COCCC1C#N] |
| 30 | 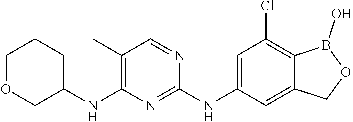 [SMILES: Cc1cnc(Nc2cc(Cl)c3c(c2)COB3O)nc1NC1CCCOC1] |
| 31 | 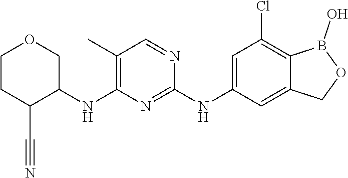 [SMILES: Cc1cnc(Nc2cc(Cl)c3c(c2)COB3O)nc1NC1COCCC1C#N] |
| 32 | 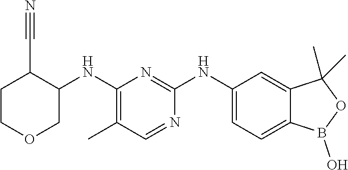 [SMILES: Cc1cnc(Nc2ccc3c(c2)C(C)(C)OB3O)nc1NC1COCCC1C#N] |
| 33 | 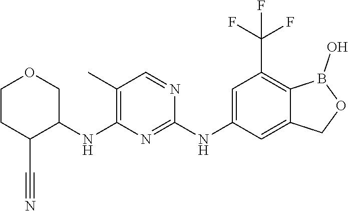 [SMILES: Cc1cnc(Nc2cc3c(c(C(F)(F)F)c2)B(O)OC3)nc1NC1COCCC1C#N] |
| 34 | 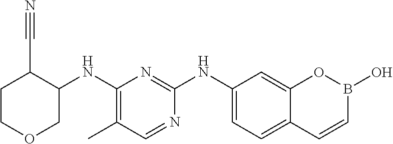 [SMILES: Cc1cnc(Nc2ccc3c(c2)OB(O)C=C3)nc1NC1COCCC1C#N] |
| 35 | 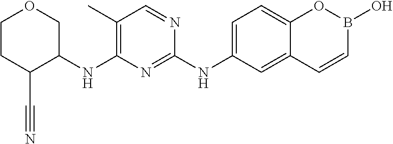 [SMILES: Cc1cnc(Nc2ccc3c(c2)C=CB(O)O3)nc1NC1COCCC1C#N] |
| 36 | 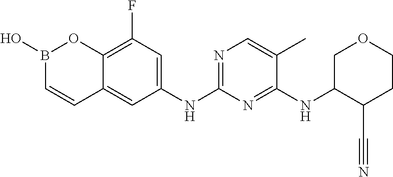 [SMILES: Cc1cnc(Nc2cc(F)c3c(c2)C=CB(O)O3)nc1NC1COCCC1C#N] |
| 37 | 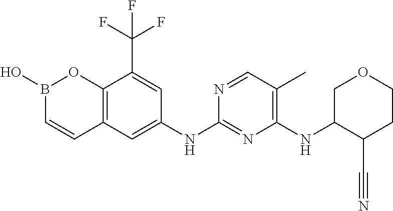 [SMILES: Cc1cnc(Nc2cc3c(c(C(F)(F)F)c2)OB(O)C=C3)nc1NC1COCCC1C#N] |
| 38 | 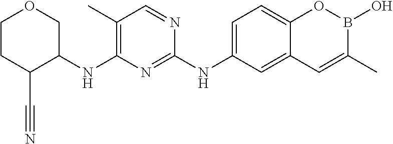 [SMILES: CC1=Cc2cc(Nc3ncc(C)c(NC4COCCC4C#N)n3)ccc2OB1O] |
| 39 | 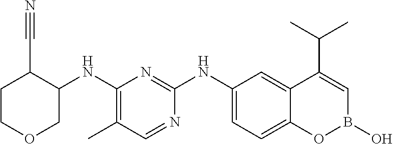 [SMILES: Cc1cnc(Nc2ccc3c(c2)C(C(C)C)=CB(O)O3)nc1NC1COCCC1C#N] |
| 40 | 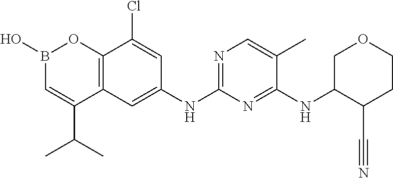 [SMILES: Cc1cnc(Nc2cc(Cl)c3c(c2)C(C(C)C)=CB(O)O3)nc1NC1COCCC1C#N] |
| 41 | 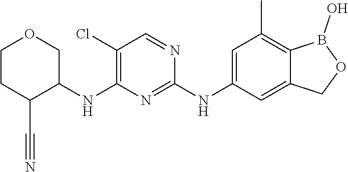 [SMILES: Cc1cc(Nc2ncc(Cl)c(NC3COCCC3C#N)n2)cc2c1B(O)OC2] |
| 42 | 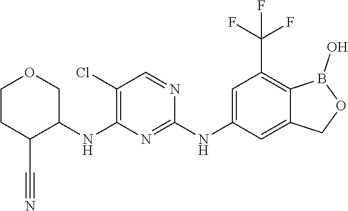 [SMILES: N#CC1CCOCC1Nc1nc(Nc2cc3c(c(C(F)(F)F)c2)B(O)OC3)ncc1Cl] |
| 43 | 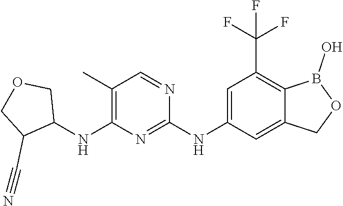 [SMILES: Cc1cnc(Nc2cc3c(c(C(F)(F)F)c2)B(O)OC3)nc1NC1COCC1C#N] |
| 44 | 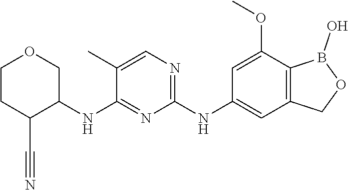 [SMILES: COc1cc(Nc2ncc(C)c(NC3COCCC3C#N)n2)cc2c1B(O)OC2] |
| 45 | 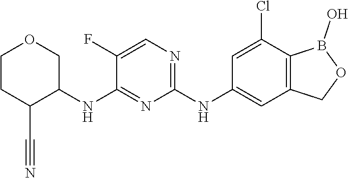 [SMILES: N#CC1CCOCC1Nc1nc(Nc2cc(Cl)c3c(c2)COB3O)ncc1F] |
| 46 | 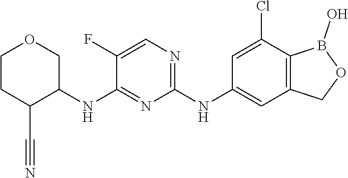 [SMILES: N#CC1CCOCC1Nc1nc(Nc2cc(Cl)c3c(c2)COB3O)ncc1F] |
| 47 | 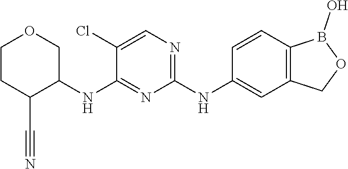 [SMILES: N#CC1CCOCC1Nc1nc(Nc2ccc3c(c2)COB3O)ncc1Cl] |
| 48 | 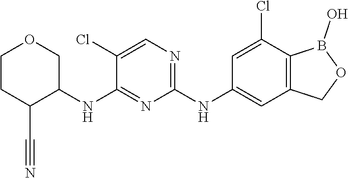 [SMILES: N#CC1CCOCC1Nc1nc(Nc2cc(Cl)c3c(c2)COB3O)ncc1Cl] |
| 49 | 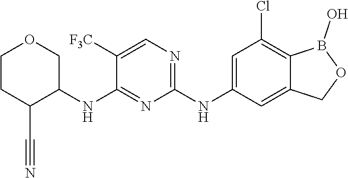 [SMILES: N#CC1CCOCC1Nc1nc(Nc2cc(Cl)c3c(c2)COB3O)ncc1C(F)(F)F] |
| 50 | 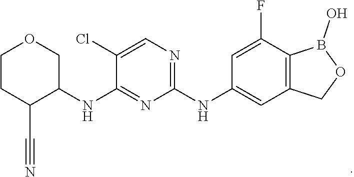 [SMILES: N#CC1CCOCC1Nc1nc(Nc2cc(F)c3c(c2)COB3O)ncc1Cl] |

#15. The compound according to any example in paragraph 10 and 14 which is a stereoisomer having trans relative stereochemistry as represented in formula (Ia) and (Ib):

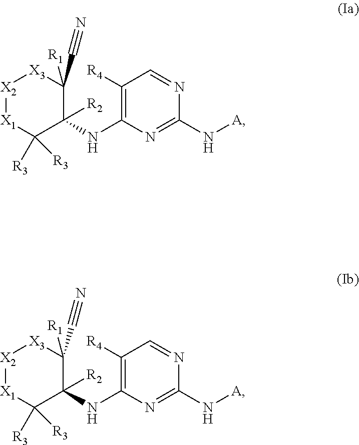

#16. The compound according to one or more of paragraphs 10 and 14, wherein the compound is a single stereoisomer having trans relative stereochemistry.

#17. The compound according to claim 16, wherein the compound has trans stereochemistry as represented by either formula (Ia) or formula (Ib).

#18. A process for preparing a compound according to any one of paragraphs 1-17.

#19. A method for treating a patient having a disease or disorder susceptible to modulation of JAK including administering a therapeutically effective amount of a compound according to any one of paragraphs 1-17.

#20. The method of paragraph 19, wherein the disease or disorder is a condition that can be ameliorated by the selective inhibition of a Janus kinase JAK 1 relative to JAK 2.

#21. The method of paragraph 19, wherein the disease or disorder is one or more of atopic dermatitis, flea allergy dermatitis, eczema, pruritus, psoriasis, psoriatic arthritis, inflammatory/autoimmune polyarthritis, Bechet's disease, pityriasis rubra pilaris, alopecia areata, discoid lupus erythematosus, vitiligo, palmoplantar pustulosis, mucocutaneous disease erythema multiforme, mycosis fungoides, graft-versus-host disease, cutaneous lupus, rheumatoid arthritis (RA), arthritis, ulcerative colitis, Crohn's disease, inflammatory bowel disease (IBD), transplant rejection, systemic lupus erythematosus (SLE), dermatomyositis, Sjogren's syndrome, dry eye disease, secondary hypereosinophilic syndrome (HES), allergy, allergic dermatitis, allergic rhinitis, asthma, vasculitis, multiple sclerosis, diabetic nephropathy, cardiovascular disease, artherosclerosis, and cancer.

#22. The method of paragraph 21, wherein the disease or disorder is one or more of atopic dermatitis, flea allergy dermatitis psoriasis, and rheumatoid arthritis.

#23. The method according to any one of paragraphs 19-22, wherein the compound is administered in an amount to perturb an immune regulatory pathway in a cell.

#24. The method of paragraph 23, wherein the perturbation results in an effect on the JAK-STAT pathway.

#25. A method of inhibiting JAK in a mammalian cell including contacting the mammalian cell with a compound any one of paragraphs 1-17.

#26. The method according to paragraph 25, wherein the mammalian cell is a cell from a subject having an inflammatory condition.

#27. A composition including a compound of any one of paragraphs 1-17 and a pharmaceutically or veterinary acceptable carrier.

#28. A combination including a compound of any one of paragraphs 1-17, and one or more other pharmaceutical or veterinary active substances.

#29. A method for treating one or more diseases or disorders of inflammation, auto-immune dysfunction, and cancer including administering to a subject in need thereof an effective amount of a compound of any one of paragraphs 1-17.

#30. The method of paragraph 29, wherein the disease or disorder is atopic dermatitis, flea allergy dermatitis, psoriasis, or rheumatoid arthritis.

#31. The method of paragraph 29 or paragraph 30, wherein the compound is administered orally, parenterally, or topically.

#32. The method of any one of paragraphs 29-31, wherein the subject is a mammal.

#33. The method of paragraph 32, wherein the mammal is selected from one or more of livestock mammals, domestic mammals, and companion animals.

#34. The method of paragraph 32, wherein the mammal is selected from one or more of humans, cattle, sheep, goats, llamas, alpacas, pigs, horses, donkeys, dogs, and cats.

#35. The method of paragraph 32, wherein the mammal is a human, dog, or cat.

#36. A compound of any one of paragraphs 1-17 for use in medicine.

#37. Use of a compound of any one of paragraphs 1-17 for the manufacture of a medicament for the treatment of one or more diseases or disorder of inflammation, auto-immune dysfunction, and cancer.

#38. The use according to paragraph 37, wherein the disease or disorder is atopic dermatitis, flea allergy dermatitis, psoriasis, or rheumatoid arthritis.

#39. The use according to paragraph 37, wherein the disease or a disorder is ameliorated by the selective inhibition of a Janus kinase JAK 1 relative to JAK 2.

#40. Use of a compound of any one of paragraphs 1-17 and a second active agent in the manufacture of a medicament for the treatment of a disease or a disorder that can be ameliorated by the selective inhibition of a Janus kinase JAK 1 relative to JAK 2.

#41. Use of a compound of any one of paragraphs 1-17 for the treatment of one or more diseases or disorders of inflammation, auto-immune dysfunction, and cancer.

#42. The use according to paragraph 41, wherein the disease or disorder is atopic dermatitis, flea allergy dermatitis, psoriasis, or rheumatoid arthritis.

Having thus described in detail various embodiments of the present invention, it is to be understood that the invention defined by the above paragraphs is not to be limited to particular details set forth in the above description as many apparent variations thereof are possible without departing from the spirit or scope of the present invention.

1. A compound of formula I: 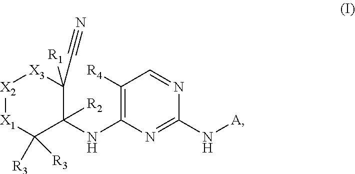
or a pharmaceutically acceptable salt or a stereoisomer or tautomer thereof, wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R₁ is selected from the group consisting of hydrogen, halogen (when X₃ is —C(R⁶)(R⁷)), hydroxy, C₁₋₆ alkyl, C₃₋₇ cycloalkyl, C₁₋₆ alkyl(oxy), and C₃₋₇ cycloalkyl(oxy), wherein the last four members of the of the above-mentioned R¹ group may optionally be substituted independently of one another with one or more halogen;
R₂ is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R₃ is independently selected from the group consisting of hydrogen, halogen (when X₁ is —C(R⁶)(R⁷)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of: hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl;
A is selected from the group consisting of benzo[c][1,2]oxaborol-1(3H)-ol, 3,4-dihydro-1 Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇: 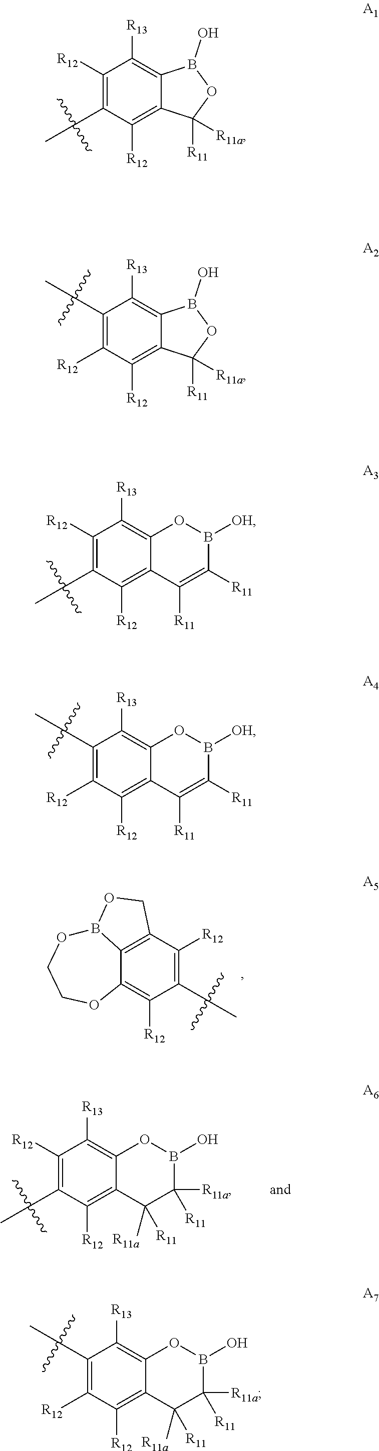
R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl); wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

2. (canceled)

3. The compound according to claim 1, wherein
X₁ is selected from the group consisting of —O—, and —C(R⁶)(R⁷)—;
X₂ is selected from the group consisting of —O—, and —C(R⁶)(R⁷)—, or
X₂ comprises a bond between X₁ and X₃;
X₃ is —C(R⁶)(R⁷)—, except X₁ and X₂ cannot both be —O—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of: hydrogen, halogen, C₁-C₃ alkyl, and C₁-C₃ haloalkyl;
R⁶ and R⁷ are independently selected from the group consisting of hydrogen, —C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring;
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

4. The compound according to claim 1, wherein
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

5. The compound according to claim 1, wherein
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

6. A compound of formula (Ia) or (Ib): 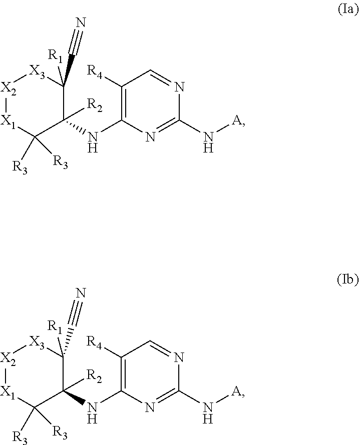
or a pharmaceutically acceptable salt or a stereoisomer or a tautomer thereof
wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or
X₂ comprises a bond between X₁ and X₃;
X₃ is selected from the group consisting of —O—, —N(R⁵)—, and —C(R⁶)(R⁷)—, except X₁ and X₂ cannot both be —O—, and X₂ and X₃ cannot both be —O—;
R¹ is selected from the group consisting of hydrogen, halogen (when X₃ is —C(R⁷)(R⁸)—), hydroxy, C₁₋₆ alkyl, C₃₋₇ cycloalkyl, C₁₋₆ alkyl(oxy), and C₃₋₇ cycloalkyl(oxy), wherein the last four members of the of the above-mentioned R¹ group may optionally be substituted independently of one another with one or more halogen;
R² is selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R² group may optionally be substituted independently of one another with one or more halogen;
each R³ is independently selected from the group consisting of hydrogen, halogen (when X₁ is —C(R⁷)(R⁸)), C₁₋₆ alkyl, C₃₋₇ cycloalkyl, C₁₋₆ alkyl(oxy), and C₃₋₇ cycloalkyl(oxy), wherein the last four members of the above-mentioned R³ group may optionally be substituted independently of one another with one or more halogen;
R₄ is selected from the group consisting of hydrogen, halogen, C₁-C₃ alkyl, C₂-C₃ alkenyl, C₂-C₃ alkynyl, —CHO, cyclopropyl, C₁-C₃ haloalkyl, C₂-C₃ haloalkenyl, C₂-C₃ haloalkynyl, and partially or fully halogenated cyclopropyl;
A is selected from the group consisting of benzo[c][1,2]oxaborol-1(3H)-ol, 3,4-dihydro-1 Hbenzo[c][1,2]oxaborinin-1-ol, and 2H-benzo[e][1,2]oxaborinin-2-ol, 7,8 dihydro-2H-1,6,9-trioxa-9-borabenzo[cd] azuline or a derivative thereof, such as a moiety selected from any one of A₁-A₇: 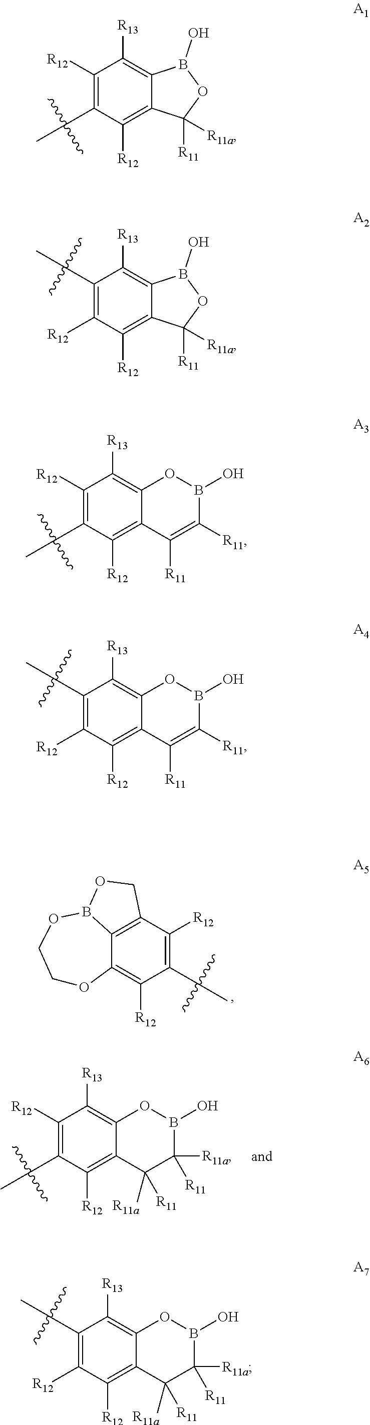
R⁵ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alky(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the above-mentioned members of the R⁵ group, except hydrogen, may optionally be substituted independently of one another by one or more substituents selected from the group consisting of C₁₋₆ alkyl and halogen, except halogen may not be adjacent to a heteroatom;
R⁶ and R⁷ are independently selected from the group consisting of hydrogen, halogen, hydroxy, —N(R⁸)(R⁹), —O(R¹⁰), C₁₋₆ alkyl, C₃₋₇cycloalkyl, G-s alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fifteen members of the above-mentioned R⁶ and R⁷ groups may optionally be substituted independently of one another by one or more substituents selected from the group consisting of halogen and hydroxy, and wherein R⁶ and R⁷ may be linked together to form a ring;
R⁸ and R⁹ are independently selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last fourteen members of the above-mentioned R⁸ and R⁹ groups may optionally be substituted independently of one another by one or more halogen, or R⁸ and R⁹ may be linked together to form a ring;
R¹⁰ is selected from the group consisting of hydrogen, C₁₋₆ alkyl, C₃₋₇ cycloalkyl, C₁₋₆ alkyl(oxy), C₃₋₇ cycloalkyl(oxy), —COO—C₁₋₆ alkyl, —COO—C₃₋₇-cycloalkyl, —(C₁₋₃-alkyl)-(C₃₋₆-cycloalkyl), —(C₂₋₃-alkyoxyl)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), -heteroaryl, and —(C₁₋₃-alkyl)-(heteroaryl), wherein the last thirteen members of the above-mentioned R¹⁰ group may optionally be substituted independently of one another by one or more substituents selected from the group consisting of halogen and hydroxy, except that said optional substitution may not be geminal;
each R¹¹ is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R^{11a} is independently selected from the group consisting of hydrogen, halogen, C₁₋₆ alkyl, C₃₋₇ cycloalkyl, 4-7-membered heterocycloalkyl, C₁₋₆ alkyl(oxy), and C₃₋₇ cycloalkyl(oxy), wherein the last four members of the above-mentioned R¹¹ group may optionally be substituted independently of one another by one or more substituents selected from the group consisting of halogen and hydroxy, except when R¹¹ is adjacent to oxygen, R¹¹ is not halogen, and two adjacent R¹¹ may be connected to provide a fused cycloalkyl, such as cyclopropyl;
each R¹² is independently selected from the group consisting of hydrogen, halogen, C₁₋₆ alkyl, and C₃₋₇ cycloalkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), —(C₂₋₃-alkyl(oxy)-(C₃₋₆-cycloalkyl), 4-7-membered heterocycloalkyl, —(C₁₋₃-alkyl)-(4-7-membered heterocycloalkyl), —N—(R⁵)(R⁵), —SO₂—(R⁵), —SO₂—(R⁵)(R⁵), —S(O)—(R⁵), and —S—(R⁵), wherein the last fourteen members of the above-mentioned R¹³ group may optionally be substituted independently of one another by one or more substituents selected from the group consisting of halogen and hydroxy.

7. The compound according to claim 6, wherein
X₁ is selected from the group consisting of —O—, —N(R⁵)—, and —C(R⁶)(R⁷)—;
X₂ is selected from the group consisting of —O—, —N(R⁵)—, and —C(R⁶)(R⁷)—, or X₂ comprises a bond between X₁ and X₃, wherein X₁ and X₂ cannot both be —O—;
X₃ is —C(R⁶)(R⁷)—;
R¹ is selected from the group consisting of hydrogen, C₁₋₆ alkyl, and C₃₋₇ cycloalkyl, wherein the last two members of the above-mentioned group may optionally be substituted independently of one another with one or more halogen;
R² is selected from the group consisting of hydrogen, C₁₋₆ alkyl, and C₃₋₇ cycloalkyl, wherein the last two members of the above-mentioned R² group may optionally be substituted independently of one another with one or more halogen;
each R³ is independently selected from the group consisting of hydrogen, C₁₋₆ alkyl, and C₃₋₇ cycloalkyl, wherein the last two members of the above-mentioned R³ group may optionally be substituted independently of one another with one or more halogen;
R₄ is selected from the group consisting of hydrogen, halogen, C₁-C₃ alkyl, and C₁-C₃ haloalkyl;
R⁶ and R⁷ are independently selected from the group consisting of hydrogen, C₁₋₆ alkyl, C₃₋₇ cycloalkyl, C₁₋₆ alkyl(oxy), and C₃₋₇ cycloalkyl(oxy), wherein the last four members of the above-mentioned R⁶ and R⁷ groups may optionally be substituted independently of one another by one or more substituents selected from the group consisting of halogen and hydroxy, and R⁶ and R⁷ may be linked together to form a ring; and
each R¹² is independently selected from the group consisting of hydrogen, halogen, and C₁₋₆ alkyl; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

8. The compound according to claim 6, wherein
X₁ is selected from the group consisting of —O—, —N(R⁵)—, and —C(R⁶)(R⁷)—;
X₂ is selected from the group consisting of —O— and —C(R⁶)(R⁷)—, or X₂ comprises a bond between X₁ and X₃, except X₁ and X₂ may not both be —O—;
X₃ is —C(R⁶)(R⁷)—;
R¹ is selected from the group consisting of hydrogen and C₁₋₆ alkyl;
R² is selected from the group consisting of hydrogen and C₁₋₆ alkyl;
each R³ is independently selected from the group consisting of hydrogen and C₁₋₆ alkyl;
R₄ is selected from the group consisting of hydrogen, halogen, C₁-C₃ alkyl, and C₁-C₃ haloalkyl;
R⁶ and R⁷ are independently selected from the group consisting of hydrogen and C₁₋₆ alkyl, wherein the C₁₋₆ alkyl may optionally be substituted with one or more of halogen;
R¹² is hydrogen; and
each R¹³ is independently selected from the group consisting of hydrogen, halogen, cyano, C₁₋₆ alkyl, C₃₋₇ cycloalkyl, C₁₋₆ alkyl(oxy), C₃₋₇ cycloalkyl(oxy), —(C₁₋₃-alkyl)-(C₃₋₆-cycloalkyl), —(C₂₋₃-alkyl(oxy)-(C₂₋₆-alkyl), and 4-7-membered heterocycloalkyl, wherein the last seven members of the above-mentioned R¹³ group may optionally be substituted independently of one another by one or more substituents selected from the group consisting of halogen and hydroxy.

9. The compound according to claim 6, wherein
X₂ is —C(R⁶)(R⁷);
X₃ is —C(R⁶)(R⁷)—;
R¹, R², and R³ are hydrogen;
R₄ is selected from the group consisting of halogen, C₁-C₃ alkyl, and C₁-C₃ haloalkyl;
R⁶ and R⁷ are independently selected from the group consisting of hydrogen and C₁₋₆ alkyl, wherein C₁₋₆ alkyl may optionally be substituted with one or more of halogen;
each R¹¹ is independently selected from the group consisting of hydrogen, halogen, C₁₋₆ alkyl, and C₃₋₇ cycloalkyl, wherein the last two substituents may optionally be substituted independently of one another by one or more halogen;
each R^{11a} is independently selected from the group consisting of hydrogen, halogen, C₁₋₆ alkyl, and C₃₋₇ cycloalkyl, wherein the last two substituents may optionally be substituted independently of one another by one or more halogen;
R¹² is hydrogen; and
each R¹³ is independently selected from the group consisting of hydrogen, halogen, cyano, C₁₋₆ alkyl, C₃₋₇ cycloalkyl, and C₁₋₆ alkyl(oxy), wherein the last three members of the above-mentioned R¹³ group may optionally be substituted independently of one another by one or more halogen.

10. A compound selected from Table 1:
TABLE 1

| Example | |
|---|---|
| Number | Structure |
| 1 | 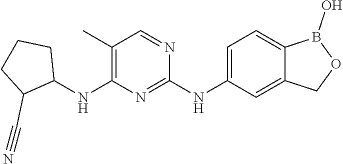 [SMILES: Cc1cnc(Nc2ccc3c(c2)COB3O)nc1NC1CCCC1C#N] |
| 2 | 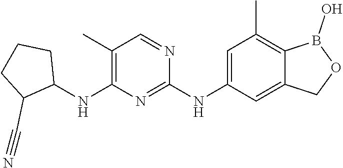 [SMILES: Cc1cnc(Nc2cc(C)c3c(c2)COB3O)nc1NC1CCCC1C#N] |
| 3 | 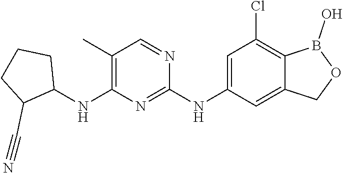 [SMILES: Cc1cnc(Nc2cc(Cl)c3c(c2)COB3O)nc1NC1CCCC1C#N] |
| 4 | 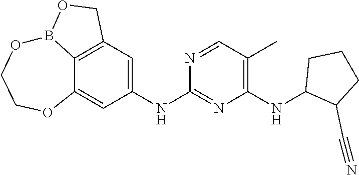 [SMILES: Cc1cnc(Nc2cc3c4c(c2)OCCOB4OC3)nc1NC1CCCC1C#N] |
| 5 | 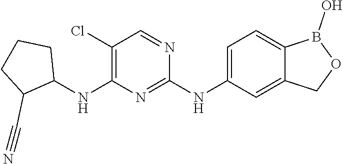 [SMILES: N#CC1CCCC1Nc1nc(Nc2ccc3c(c2)COB3O)ncc1Cl] |
| 6 | 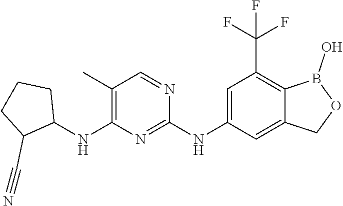 [SMILES: Cc1cnc(Nc2cc3c(c(C(F)(F)F)c2)B(O)OC3)nc1NC1CCCC1C#N] |
| 7 | 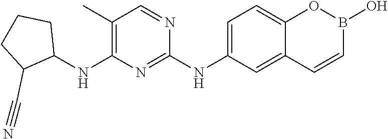 [SMILES: Cc1cnc(Nc2ccc3c(c2)C=CB(O)O3)nc1NC1CCCC1C#N] |
| 8 | 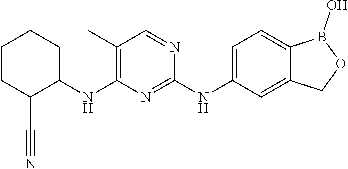 [SMILES: Cc1cnc(Nc2ccc3c(c2)COB3O)nc1NC1CCCCC1C#N] |
| 9 | 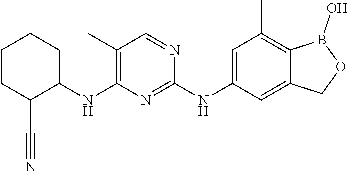 [SMILES: Cc1cnc(Nc2cc(C)c3c(c2)COB3O)nc1NC1CCCCC1C#N] |
| 10 | 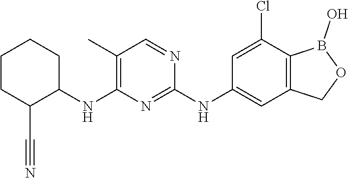 [SMILES: Cc1cnc(Nc2cc(Cl)c3c(c2)COB3O)nc1NC1CCCCC1C#N] |
| 11 | 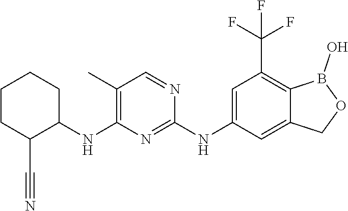 [SMILES: Cc1cnc(Nc2cc3c(c(C(F)(F)F)c2)B(O)OC3)nc1NC1CCCCC1C#N] |
| 12 | 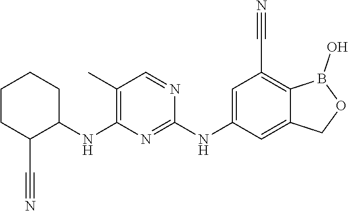 [SMILES: Cc1cnc(Nc2cc(C#N)c3c(c2)COB3O)nc1NC1CCCCC1C#N] |
| 13 | 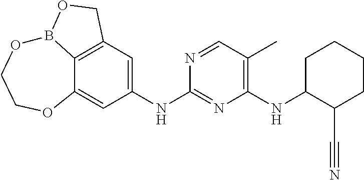 [SMILES: Cc1cnc(Nc2cc3c4c(c2)OCCOB4OC3)nc1NC1CCCCC1C#N] |
| 14 | 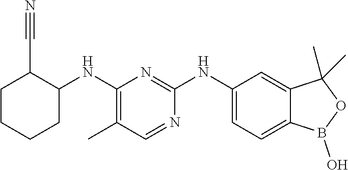 [SMILES: Cc1cnc(Nc2ccc3c(c2)C(C)(C)OB3O)nc1NC1CCCCC1C#N] |
| 15 | 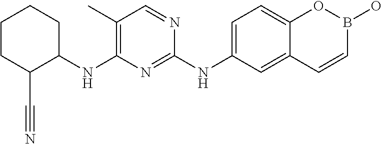 [SMILES: Cc1cnc(Nc2ccc3c(c2)C=CB(O)O3)nc1NC1CCCCC1C#N] |
| 16 | 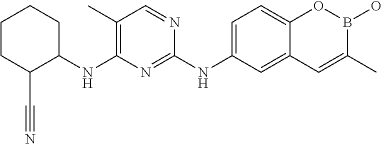 [SMILES: CC1=Cc2cc(Nc3ncc(C)c(NC4CCCCC4C#N)n3)ccc2OB1O] |
| 17 | 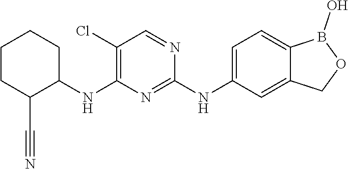 [SMILES: N#CC1CCCCC1Nc1nc(Nc2ccc3c(c2)COB3O)ncc1Cl] |
| 18 | 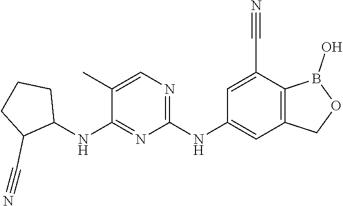 [SMILES: Cc1cnc(Nc2cc(C#N)c3c(c2)COB3O)nc1NC1CCCC1C#N] |
| 19 | 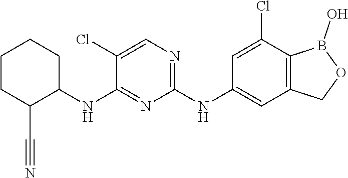 [SMILES: N#CC1CCCCC1Nc1nc(Nc2cc(Cl)c3c(c2)COB3O)ncc1Cl] |
| 20 | 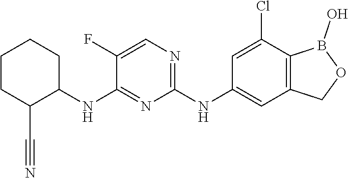 [SMILES: N#CC1CCCCC1Nc1nc(Nc2cc(Cl)c3c(c2)COB3O)ncc1F] |
| 21 | 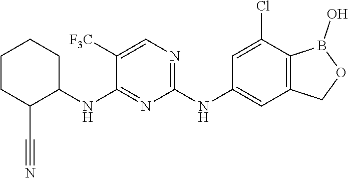 [SMILES: N#CC1CCCCC1Nc1nc(Nc2cc(Cl)c3c(c2)COB3O)ncc1C(F)(F)F] |
| 22 | 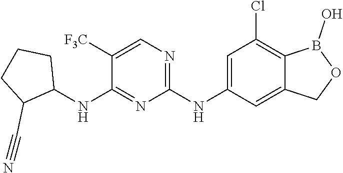 [SMILES: N#CC1CCCC1Nc1nc(Nc2cc(Cl)c3c(c2)COB3O)ncc1C(F)(F)F] |
| 23 | 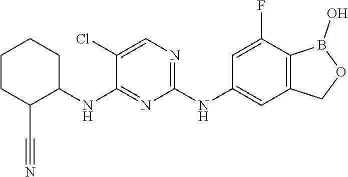 [SMILES: N#CC1CCCCC1Nc1nc(Nc2cc(F)c3c(c2)COB3O)ncc1Cl] |
| 24 | 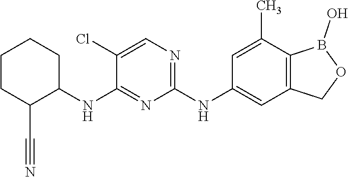 [SMILES: Cc1cc(Nc2ncc(Cl)c(NC3CCCCC3C#N)n2)cc2c1B(O)OC2] |
| 25 | 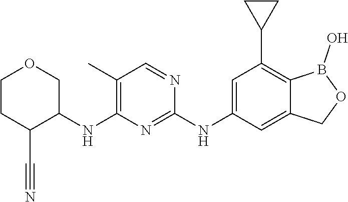 [SMILES: Cc1cnc(Nc2cc3c(c(C4CC4)c2)B(O)OC3)nc1NC1COCCC1C#N] |
| 26 | 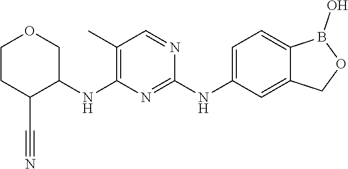 [SMILES: Cc1cnc(Nc2ccc3c(c2)COB3O)nc1NC1COCCC1C#N] |
| 27 | 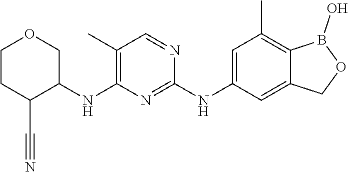 [SMILES: Cc1cnc(Nc2cc(C)c3c(c2)COB3O)nc1NC1COCCC1C#N] |
| 28 | 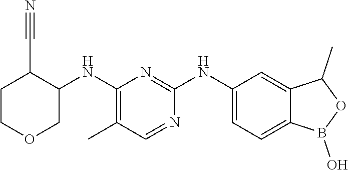 [SMILES: Cc1cnc(Nc2ccc3c(c2)C(C)OB3O)nc1NC1COCCC1C#N] |
| 29 | 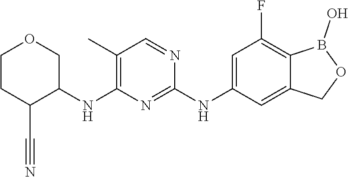 [SMILES: Cc1cnc(Nc2cc(F)c3c(c2)COB3O)nc1NC1COCCC1C#N] |
| 30 | 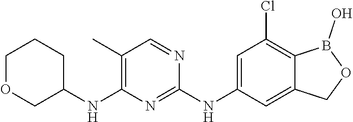 [SMILES: Cc1cnc(Nc2cc(Cl)c3c(c2)COB3O)nc1NC1CCCOC1] |
| 31 | 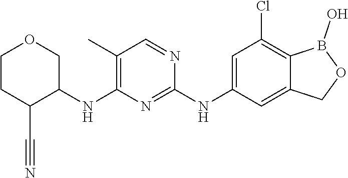 [SMILES: Cc1cnc(Nc2cc(Cl)c3c(c2)COB3O)nc1NC1COCCC1C#N] |
| 32 | 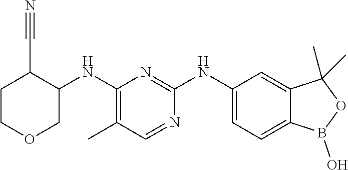 [SMILES: Cc1cnc(Nc2ccc3c(c2)C(C)(C)OB3O)nc1NC1COCCC1C#N] |
| 33 | 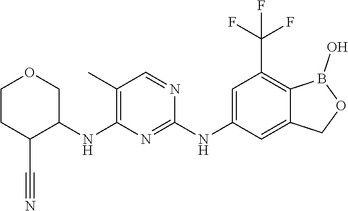 [SMILES: Cc1cnc(Nc2cc3c(c(C(F)(F)F)c2)B(O)OC3)nc1NC1COCCC1C#N] |
| 34 | 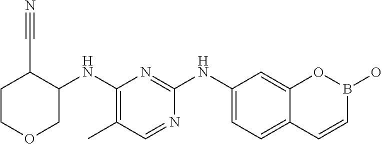 [SMILES: Cc1cnc(Nc2ccc3c(c2)OB(O)C=C3)nc1NC1COCCC1C#N] |
| 35 | 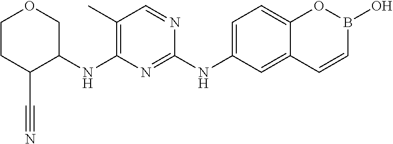 [SMILES: Cc1cnc(Nc2ccc3c(c2)C=CB(O)O3)nc1NC1COCCC1C#N] |
| 36 | 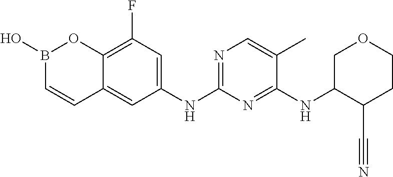 [SMILES: Cc1cnc(Nc2cc(F)c3c(c2)C=CB(O)O3)nc1NC1COCCC1C#N] |
| 37 | 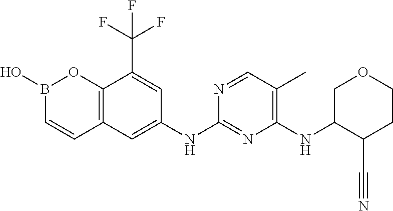 [SMILES: Cc1cnc(Nc2cc3c(c(C(F)(F)F)c2)OB(O)C=C3)nc1NC1COCCC1C#N] |
| 38 | 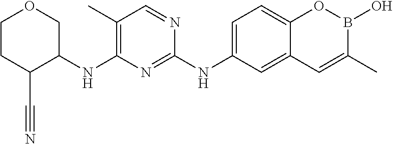 [SMILES: CC1=Cc2cc(Nc3ncc(C)c(NC4COCCC4C#N)n3)ccc2OB1O] |
| 39 | 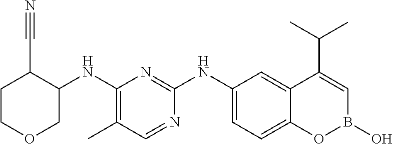 [SMILES: Cc1cnc(Nc2ccc3c(c2)C(C(C)C)=CB(O)O3)nc1NC1COCCC1C#N] |
| 40 | 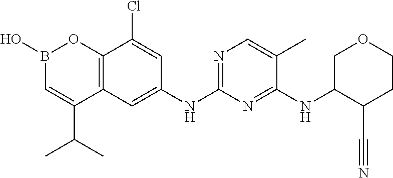 [SMILES: Cc1cnc(Nc2cc(Cl)c3c(c2)C(C(C)C)=CB(O)O3)nc1NC1COCCC1C#N] |
| 41 | 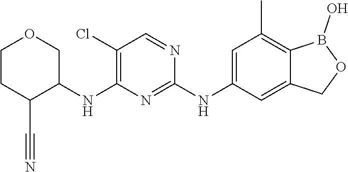 [SMILES: Cc1cc(Nc2ncc(Cl)c(NC3COCCC3C#N)n2)cc2c1B(O)OC2] |
| 42 | 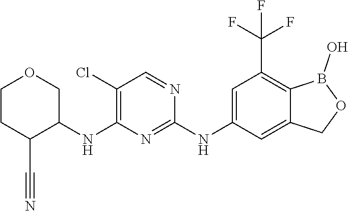 [SMILES: N#CC1CCOCC1Nc1nc(Nc2cc3c(c(C(F)(F)F)c2)B(O)OC3)ncc1Cl] |
| 43 | 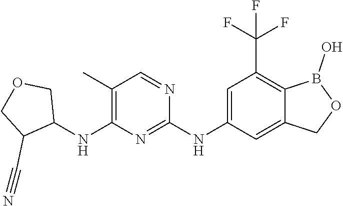 [SMILES: Cc1cnc(Nc2cc3c(c(C(F)(F)F)c2)B(O)OC3)nc1NC1COCC1C#N] |
| 44 | 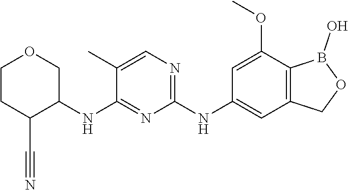 [SMILES: COc1cc(Nc2ncc(C)c(NC3COCCC3C#N)n2)cc2c1B(O)OC2] |
| 45 | 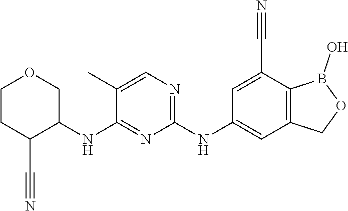 [SMILES: Cc1cnc(Nc2cc(C#N)c3c(c2)COB3O)nc1NC1COCCC1C#N] |
| 46 | 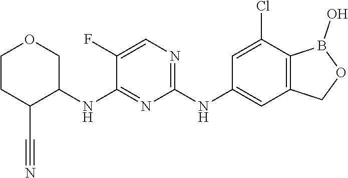 [SMILES: N#CC1CCOCC1Nc1nc(Nc2cc(Cl)c3c(c2)COB3O)ncc1F] |
| 47 | 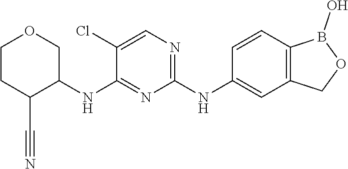 [SMILES: N#CC1CCOCC1Nc1nc(Nc2ccc3c(c2)COB3O)ncc1Cl] |
| 48 | 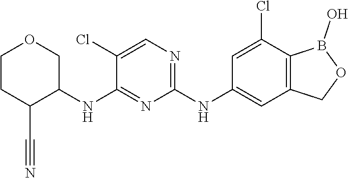 [SMILES: N#CC1CCOCC1Nc1nc(Nc2cc(Cl)c3c(c2)COB3O)ncc1Cl] |
| 49 | 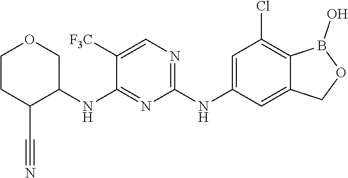 [SMILES: N#CC1CCOCC1Nc1nc(Nc2cc(Cl)c3c(c2)COB3O)ncc1C(F)(F)F] |
| 50 | 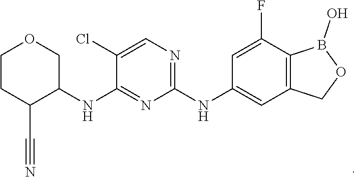 [SMILES: N#CC1CCOCC1Nc1nc(Nc2cc(F)c3c(c2)COB3O)ncc1Cl] |

11-14. (canceled)

15. The compound according to claim 10 which is a stereoisomer having trans relative stereochemistry as represented in formula (Ia) and (Ib):

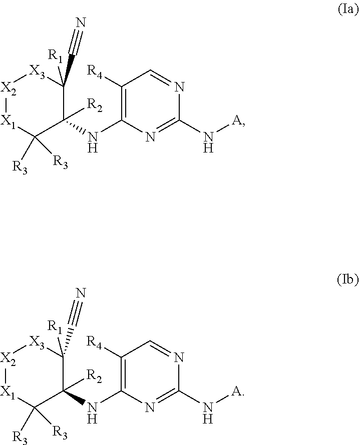

16. The compound according to claim 10, wherein the compound is a single stereoisomer having trans relative stereochemistry.

17. (canceled)

18. (canceled)

19. A method for treating a patient having a disease or disorder susceptible to modulation of JAK-STAT pathway in a mammalian cell comprising administering a therapeutically effective amount of a compound according to claim 1.

20. The method of claim 19, wherein the disease or disorder is a condition that can be ameliorated by the selective inhibition of a Janus kinase JAK 1 relative to JAK 2.

21. The method of claim 19, wherein the disease or disorder is one or more of atopic dermatitis, flea allergy dermatitis, eczema, pruritus, psoriasis, psoriatic arthritis, inflammatory/autoimmune polyarthritis, Bechet's disease, pityriasis rubra pilaris, alopecia areata, discoid lupus erythematosus, vitiligo, palmoplantar pustulosis, mucocutaneous disease erythema multiforme, mycosis fungoides, graft-versus-host disease, cutaneous lupus, rheumatoid arthritis (RA), arthritis, ulcerative colitis, Crohn's disease, inflammatory bowel disease (IBD), transplant rejection, systemic lupus erythematosus (SLE), dermatomyositis, Sjogren's syndrome, dry eye disease, secondary hypereosinophilic syndrome (HES), allergy, allergic dermatitis, allergic rhinitis, asthma, vasculitis, multiple sclerosis, diabetic nephropathy, cardiovascular disease, artherosclerosis, and cancer.

22-26. (canceled)

27. A composition comprising a compound of claim 1 and a pharmaceutically or veterinary acceptable carrier.

28. A combination comprising a compound of claim 1, and one or more other pharmaceutical or veterinary active substances.

29. (canceled)

30. The method of claim 19, wherein the disease or disorder is atopic dermatitis, flea allergy dermatitis, psoriasis, or rheumatoid arthritis.

31. The method of claim 19, wherein the compound is administered orally, parenterally, or topically.

32-33. (canceled)

34. The method of claim 19, wherein the patient is a mammal is selected from one or more of humans, cattle, sheep, goats, llamas, alpacas, pigs, horses, donkeys, dogs, and cats.

35-42. (canceled)
